SEQUENCE LISTING (9 sequences)

SEQ ID NO: 1 - protein, 461 aa, Homo sapiens
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 2 - protein, 295 aa, Artificial Sequence
  Description of Artificial Sequence Synthetic polypeptide
MRGSHHHHHHGMASMTGGQQMGRDLYDDDDKDLVPRGSPTLGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 3 - protein, 13 aa, Artificial Sequence
  Description of Artificial Sequence Synthetic peptide
KDHQLLRYLLDKD

SEQ ID NO: 4 - protein, 13 aa, Artificial Sequence
  Description of Artificial Sequence Synthetic peptide
ERHKILHRLLQEG

SEQ ID NO: 5 - DNA, 24 bp, Artificial Sequence
  Description of Artificial Sequence Synthetic primer
ggcagccagggctccggggaaggc

SEQ ID NO: 6 - DNA, 27 bp, Artificial Sequence
  Description of Artificial Sequence Synthetic primer
ggtggatcccctggactggggcttatc

SEQ ID NO: 7 - DNA, 45 bp, Artificial Sequence
  Description of Artificial Sequence Synthetic primer
gacgatgacgataaggatctggttccgcgtggatccccaaccctt

SEQ ID NO: 8 - DNA, 45 bp, Artificial Sequence
  Description of Artificial Sequence Synthetic primer
aagggttggggatccacgcggaaccagatccttatcgtcatcgtc

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2011/0060580 A1
(43) Pub. Date: Mar. 10, 2011

(54) LXR LIGAND BINDING DOMAIN (LXR LBD) CRYSTALS

(76) Inventors: Joerg Benz, Rheinfelden (DE); Bernard Gsell, Lutter (FR); Martine Stihle, Michelbach-le-bas (FR); Ralf Thoma, Grenzach-Whylen (DE); Matthew Wright, Basel (CH)

(21) Appl. No.: 12/922,201

(22) PCT Filed: Mar. 10, 2009
(86) PCT No.: PCT/EP09/01713

§ 371 Date: Sep. 13, 2010

(30) Foreign Application Priority Data
Mar. 17, 2008 (EP) 08152843.2

(51) Int. Cl. C07K 14/705; G06G 7/58

(52) U.S. Cl. 703/12; 530/350

(57) ABSTRACT

The present invention relates to co-crystals of Liver X receptor beta ligand binding domain (LXRβ-LBD) with agonists and to the three-dimensional X-ray crystal structures derived thereof.

The present invention relates to co-crystals of Liver X receptor beta ligand binding domain (LXRβ-LBD) with agonists and to the three-dimensional X-ray crystal structures derived thereof.

Liver X receptors (LXR) are members of the superfamily of nuclear receptors. These transcription factors regulate target genes through a complex series of interactions with specific DNA response elements as well as transcriptional coregulators. The binding of ligand has profound effects on these interactions and has the potential to trigger both gene activation and, in some cases, gene silencing. There are about 50 sequence-related nuclear receptors in humans and the family comprises receptors that recognize hormones, both steroidal and non-steroidal, but also receptors responding to metabolic intermediates and to xenobiotics. There are also a number of so-called orphan receptors where the natural ligand is unknown. Some of the receptors show a very specific and high affinity ligand binding, like the thyroid hormone receptors, while others have a substantially lower affinity for their ligands and are also highly promiscuous in terms of ligand selectivity. Like many of the other non-steroid hormone receptors, LXR functions as a heterodimer with the 9-cis-retinoic acid receptor (RXR) to regulate gene expression. Together with peroxisome proliferator-activated receptors (PPARs) and farnesoid X receptor (FXR) LXRs represent a subclass of so called permissive RXR heterodimers. In this subclass, the RXR heterodimers can be activated independently by either the RXR ligand, the partner's ligand or synergistically by both.

LXRs consist of two closely related receptor isoforms encoded by separate genes LXRα (NR1H3) and LXRβ (NR1H2). As expected, the largest sequence differences are located in the N-terminal domain and in the so-called hinge region connecting the DNA-binding domain (DBD) and the ligand-binding domain (LBD). LXRβ shows tissue restricted expression with the highest mRNA levels detected in the liver and to a lesser extent in the kidney, small intestine, spleen and adrenal gland. In contrast, LXRβ is ubiquitously expressed Both LXR isoforms have been shown to be activated by specific oxysterols that can be formed in vivo. Important insight into LXR biology has been obtained through the study of LXR deficient mice. Both LXRβ and LXRβ knockout mice have been described. The LXRβ null strain exhibits a striking inability to metabolize and excrete excess cholesterol when challenged with a high-cholesterol diet. The explanation appears to be an inability to up-regulate the rate-limiting enzyme in cholesterol conversion to bile acid, cholesterol 7 α-hydroxylase (CYP7A), in response to the excess cholesterol. As a consequence, the conversion of cholesterol to bile-acid that would normally occur is blunted and cholesteryl esters deposit in the liver ultimately resulting in liver-failure. In contrast, the LXRβ knockout strain maintains its natural resistance to a high cholesterol diet. These important findings not only prove an important function of LXRβ in rodent cholesterol metabolism, but also suggest that the LXR dependent regulation of CYP7A is LXR-subtype selective. The CYP7A LXR response element is not well conserved between rodents and man. LXRs are therefore not expected to be main regulators of cholesterol conversion to bile-acids in humans. This notion is supported by results from in vitro assays using cultured human cells. However, more recently, LXRs have been shown to regulate also several other genes involved in cholesterol and lipid homeostasis. Prominent examples are the phospholipid/cholesteryl ester transporter ABCA1, ABCG1 and the SREBP1c gene that, in turn, induces fatty acid synthesizing enzymes. Increasing insight into the involvement of LXRs in cholesterol and fatty acid homeostasis has led to considerable interest in LXRs as targets for drug development.

Therefore, there is a need for LXRβ-LBD co-crystal forms allowing a reproducible crystallization for subsequent X-ray crystallographic analysis and structure-based drug design.

In a first aspect, the present invention provides a co-crystal of a liver X receptor beta ligand binding domain (LXRβ-LBD) with a ligand, wherein the crystal belongs to space group P4₃.

In a preferred embodiment the crystal has unit cell dimensions of a=b=58±4 Å, c=181 ±4 Å, α=β=γ=90°±3°, preferably ±2°, more preferably ±1°, most preferably α=β=γ=90°.

In another preferred embodiment the ligand is 2-(4-{[2-(3-Chloro-phenyl)-5-methyl-oxazol-4-ylmethyl]-ethyl-amino}-phenyl)-1,1,1,3,3,3-hexafluoro-propan-2-ol.

In a second aspect, the present invention provides a co-crystal of a liver X receptor beta ligand binding domain (LXRβ-LBD) with a ligand, wherein the crystal belongs to space group P4₁2₁2.

In a preferred embodiment the crystal has unit cell dimensions of a=b=92 ±4 Å, c=273 ±4 Å, α=β=γ=90°, preferably ±2°, more preferably ±1°, most preferably α=β=γ=90°.

In another preferred embodiment the ligand is 1,1,1,3,3,3-Hexafluoro-2-{2-methyl-1-[5-methyl-2-(3-trifluoromethyl-phenyl)-oxazol-4-ylmethyl]-1H-indol-5-yl}-propan-2-ol.

In a third aspect, the present invention provides a co-crystal of a liver X receptor beta ligand binding domain (LXRβ-LBD) with a ligand, wherein the crystal belongs to space group P 1 .

In a preferred embodiment the crystal has unit cell dimensions of a=47±4 Å, b=98 ±4 Å, c=114 ±4 Å, α=74°±3°, β=98°±3°, γ=79°±3° preferably ±2°, more preferably ±1°, most preferably α=74°, β=98°, γ=79°.

In another preferred embodiment, the ligand is (R,S)-Benzenesulfonyl-(6-chloro-2,3,4, 9-tetrahydro-1H-carbazol-2-yl)-fluoro-acetic acid methyl ester.

In a further preferred embodiment of the co-crystals of the present invention, the LXRβ-LBD polypeptide is a polypeptide comprising a sequence having a similarity to the ligand binding domain of a polypeptide of Seq. Id. No. 1 of at least 80%, preferably at least 85%, more preferably at least 90%, even more preferably at least 95%, most preferably 100%.

In yet another preferred embodiment, the LXRβ-LBD polypeptide comprises amino acids 213-461 of Seq. Id. No.1 .

In a fourth aspect, the present invention provides a method for co-crystallizing an LXRβ-LBD polypeptide with a compound that binds to the ligand binding site of said polypeptide. Said method comprises the steps:

a) providing an aqueous solution of the polypeptide,
b) adding a molar excess of the ligand to the aqueous solution of the polypeptide, and
c) growing crystals.

In a preferred embodiment of the method of the present invention, the aqueous solution comprise 5% to 30% (w/v) PEG, wherein the PEG has an average molecular weight of 200 Da to 20 kDa, preferably 200 Da to 5 kDa, more preferably 3,35 kDa.

In a further preferred embodiment of the method of the present invention, the aqueous solution comprises 0 M to 1 M Bis-Tris pH 5.5, 0 M to 0.5 M magnesium chloride, and 0 M to 0.5 M ammonium sulfate.

In yet a further preferred embodiment of the method of the present invention, the aqueous solution comprises a molar excess of a co-activator peptide.

Co-crystals of the present invention can be grown by a number of techniques including batch crystallization, vapour diffusion (either by sitting drop or hanging drop) and by microdialysis. Seeding of the crystals in some instances is required to obtain X-ray quality crystals. Standard micro- and/or macroseeding of crystals may therefore be used.

In a preferred embodiment of the invention, co-crystals are grown by vapor diffusion. In this method, the polypeptide solution is allowed to equilibrate in a closed container with a larger aqueous reservoir having a precipitant concentration optimal for producing crystals. Generally, less than about 10 μl of substantially pure polypeptide solution is mixed with an equal or similar volume of reservoir solution, giving a precipitant concentration about half that required for crystallization. This solution is placed as a droplet on a plastic post surrounded by reservoir, which is sealed. The sealed container is allowed to stand, from one day to one year, usually for about 2-6 weeks, until crystals grow.

The co-crystals of the present invention can be obtained by a method which comprises: providing a buffered, aqueous solution of 3.75 to 50 mg/ml of a LXRβ-LBD polypeptide, adding a molar excess of a ligand and co-activator peptide to the aqueous polypeptide solution, and growing crystals by vapor diffusion or microbatch using a buffered reservoir solution of 0% to 30% (w/v) PEG, wherein the PEG has an average molecular weight of 200 Da to 20000 Da. The PEG may be added as monomethyl ether. A preferred PEG has an average molecular weight of 500 Da to 5,000 Da. A preferred buffered reservoir solution further comprises 0 M to 1 M Bis-Tris pH 5.5, 0 M to 0.5 M magnesium chloride, and 0 M to 0.5 M ammonium sulphate. Said microbatch may be modified.

In a preferred embodiment of the method for generating a co-crystal of the LXRβ-LBD polypeptide, the method is performed in presence of a 12-15 molar excess of the ligand and 3-12 molar excess of a co-activator peptide. A preferred co-activator peptide is selected from peptides having the sequence set forth in Seq. Id. No. 3 and Seq. Id. No. 4.

Methods for obtaining the three-dimensional structure of the crystals described herein, as well as the atomic structure coordinates, are well-known in the art (see, e.g., D. E. McRee, Practical Protein Crystallography, published by Academic Press, San Diego (1993), and references cited therein).

The crystals of the invention, and particularly the atomic structure coordinates obtained therefrom, have a wide variety of uses. For example, the crystals and structure coordinates described herein are particularly useful for identifying compounds that bind to LXRβ-LBD as an approach towards developing new therapeutic agents.

The structure coordinates described herein can be used as phasing models in determining the crystal structures of additional native or mutated polypeptides, as well as the structures of co-crystals of LXRβ-LBD with bound ligand. The structure coordinates, as well as models of the three-dimensional structures obtained therefrom, can also be used to aid the elucidation of solution-based structures of native or mutated LXRβ-LBD, such as those obtained via NMR. Thus, the crystals and atomic structure coordinates of the invention provide a convenient means for elucidating the structures and functions of an LXRβ polypeptide.

The present invention also provides a method for identifying a compound that can bind to the binding site of a LXR polypeptide. Said method comprises the steps: determining a ligand binding site of a LXR polypeptide from the three dimensional model of LXRβ-LBD polypeptide using the atomic coordinates of FIG. 1, FIG. 2 or FIG. 3, ± a root mean square deviation from the backbone atoms of said amino acids of not more than 2 Å; and performing computer fitting analysis to identify a compound that can bind to the ligand binding site of the LXR polypeptide.

In a preferred embodiment, the method comprises the steps: generating a three dimensional model of an active site of a LXR polypeptide using the relative structural data coordinates of FIG. 1, 2 or 3 of residues GLN235, CYS238, ASN239, PHE243, PHE268, PHE271, THR272, LEU274, ALA275, SER278, GLU281, ILE282, PHE285, ILE309, ILE311, MET312, GLU315, THR316, ARG319, ILE327, THR328, PHE329, LEU330, PHE329, TYR335, PHE340, LEU345, PHE349, ILE353, ILE374, HIS435, GLN438, VAL439, LEU442, LEU449 and TRP457 ±a root mean square deviation from the backbone atoms of said amino acids of not more than 2 Å; and performing computer fitting analysis to identify a compound that can bind to the LXR active site.

In a further aspect, the present invention provides a co-crystal of a LXRβ-LBD containing LXRβ-LBD in a conformation defined by the coordinates of FIG. 1, FIG. 2 or FIG. 3, optionally varied by an rmsd of less than 2.0Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of LXRβ-LBD or an active binding site thereof, as defined by the structure coordinates of LXRβ-LBD described herein.

Molecular docking of large compound databases to target proteins of known or modelled 3-dimensional structure is now a common approach in the identification of new lead compounds. This “virtual screening” approach relies on fast and accurate estimation of the ligand binding mode and an estimate of ligand affinity. Typically a large database of compounds, either real or virtual is docked to a target structure and a list of the best potential ligands is produced. This ranking should be highly enriched for active compounds which may then be subject to further experimental validation.

The calculation of the ligand binding mode may be carried out by molecular docking programs which are able to dock the ligands in a flexible manner to a protein structure. The estimation of ligand affinity is typically carried out by the use of a separate scoring function. These scoring functions include energy-based approaches which calculate the molecular mechanics force field and rule-based approaches which use empirical rules derived from the analysis of a suitable database of structural information. Consensus scoring involves rescoring each ligand with multiple scoring functions and then using a combination of these rankings to generate a hit list.

### SHORT DESCRIPTION OF THE FIGURES

FIG. 1 shows the coordinates of a co-crystal of human LXRβ-LBD (amino acids 213-461 of Seq. Id. No. 1) with ligand 2-(4-{[2-(3-Chloro-phenyl)-5-methyl-oxazol-4-ylmethyl]-ethyl-amino}-phenyl)-1,1,1,3,3,3-hexafluoro-propan-2-ol; the coordinates of amino acids 220-459 of Seq. Id. No. 1 and amino acids 3 to 13 of Seq. Id. No. 3 are shown;

FIG. 2 shows the coordinates of a co-crystal of human LXRβ-LBD (amino acids 213-461 of Seq. Id. No. 1) with ligand 1,1,1,3,3,3-Hexafluoro-2-{2-methyl-1-[5-methyl-2-(3-trifluoromethyl-phenyl)-oxazol-4-ylmethyl]-1H-indol-5-yl}-propan-2-ol; the coordinates of amino acids 220-459 of Seq. Id. No. 1 and amino acids 3 to 13 of Seq. Id. No. 3 are shown and

FIG. 3 shows the coordinates of a co-crystal of human LXRβ-LBD (amino acids 213-461 of Seq. Id. No. 1) with ligand R,S)-Benzenesulfonyl-(6-chloro-2,3,4,9-tetrahydro-1H-carbazol-2-yl)-fluoro-acetic acid methyl ester; the coordinates of amino acids 220-459 of Seq. Id. No. 1 and amino acids 3 to 13 of Seq. Id. No. 4 are shown.

### EXPERIMENTAL PART

### Example 1

### Crystal structure of human LXRβ-LBD with agonist 2-(4-{[2-(3-Chloro-phenyl)-5-methyl-oxazol-4-ylmethyl]-ethyl-amino}-phenyl)-1,1,1,3,3,3-hexafluoro-propan-2-ol and co-activator peptide

Methods:

Cloning

A plasmid construct was derived to express a fusion protein in bacteria consisting of an N-terminal 6xHis tag, followed by a thrombin cleavage site and amino acids 213-460 of human LXRβ. The DNA sequence encoding aa 213-460 of human LXRβ was first amplified by PCR using a prior human LXRβ clone as template and the following primers: Beta213ForAmp=GGCAGCCAGGGCTCCGGGGAAGGC (Seq. Id. No. 5) and LXRbetaFus2R=GGTGGATC CCTGGACTGGGGCTTATC. The resulting PCR product was subcloned into the pCR-T7-Topo vector and a correct clone was verified by double-stranded DNA sequence analysis. To produce the final construct, a thrombin cleavage site was introduced by site-directed mutagenesis to introduce 18-nucleotides just downstream of the sequence encoding the 6-His tag using the following primers: EKThrombMutF=GACGATGACGATAAGGATCTGGTTCCGCGTGGAT CCCCAACCCTT (Seq. Id. No. 7) and EKThrombMutR=AAGGGTTGGGGATCCACGCGG AACCAGATCCT TATCG TCA TCGTC (Seq. Id. No. 8). The final construct, pCRT7NT-N6HisThrombin-hIARbeta213G-460E, was confirmed by DNA sequence analysis. Seq. Id. No. 2 shows the amino acid sequence encoded by the insert of pCRT7NT-N6HisThrombin-hIARbeta213G-460E.

Production and purification of recombinant human LXRβ-LBD in E.coli:

The plasmid pCRT7NT-N6HisThrombin-hLXRbeta213G-460E was transformed into chemical competent HMS174 (DE3) cells and heterologously expressed at 20 ° C. in M9Y media by induction with 0.5 mM IPTG at an optical density at 600 nm of 0.8.

Cells were resuspended in 50 mM HEPES pH 7.5, 50 mM KCl, 10 mM MgCl₂, 4 mM DIFP and the suspension supplemented with 20 tablets Roche complete protease inhibitor mix and 30 mg DNAse I per liter. After cell disruption 1% Triton X-100 was added to the lysed cells and incubated for 30 minutes at 4 ° C. The supernatant was filtered through 0.22 gm after centrifugation at 20000 ×g for 60 mM at 4 ° C. and loaded onto a Fractogel EMD Ni2⁺chelat column equilibrated with 50 mM HEPES pH 7.5, 50 mM KCl, 10 mM TCEP, 10 mM MgCl₂ and 1% Triton X-100. After a washing step with the same buffer additional washing steps with 50 mM HEPES pH 7.5, 500 mM KCl, 10 mM TCEP, 10 mM MgCl₂ and 1% Triton X-100 and with two step gradients containing 18 mM and 36 mM Imidazole in the same buffer were performed.

The protein is eluted with a imidazole gradient from 36 mM to 330 mM. Fractions are analyzed by RPC-HPLC. The N-terminal His-tag of LXRβ in the pooled fractions was removed by Thrombin cleavage within three days at 4 ° C. For stabilization of the protein 10% glycerol and 0.1% CHAPS was added to the buffer.

In a final step the LXRβ-LBD was concentrated and applied onto a Superdex S 200 GL/300 column equilibrated with 25 mM Tris pH 7.5, 75 mM NaCl, 2 mM TCEP and 0.02% NaN3.

Crystallization:

Protein used for crystallization of LXRβ-LBD together with 2-(4-{[2-(3-Chloro-phenyl)-5-methyl-oxazol-4-ylmethyl]-ethyl-amino }-phenyl)-1,1,1,3,3,3-hexafluoro-propan-2-ol has been purified as described above. The protein was incubated with ligand in a 12-fold molar excess for 2 hours at room temperature. A short co-activator peptide (KDHQLLRYLLDKD) (Seq. Id. No. 3) from SRC-1 was added in 12-fold molar excess and incubation continued overnight at 4 ° C. Prior to crystallization experiments the protein was centrifuged at 20000× g. The crystallization droplets were set up at 22 ° C. by mixing 1.5 μl of protein solution with 0.5 μl reservoir solution in vapour diffusion hanging drop experiments. Crystals appeared out of 0.2 M (NH₄)₂SO₄, 25% PEG 3350 after 1 day and grew to final size of 0.15 mm×0.15 mm×0.05 mm within 2 days.

Crystals were harvested with paraffin oil as cryoprotectant and then flash frozen in a 100 K N2 stream. Diffraction images were collected at a temperature of 100 K at the beamline X06SA of the Swiss Light Source and processed with the programs DENZO and SCALEPACK yielding data to a resolution of 3.2 Å. Standard crystallographic programs from the CCP4 software suite were used to determine the structure by molecular replacement using an in-house LXRβ-LBD structure as search model. Refinement and model building cycles were performed with REFMAC and MOLOC, respectively (Table 1).

Results:

Crystals belong to space group P4₃ with cell axes a=58.0 Å, b=58.0 Å, c=181.8 Å, α=β=γ=90° and contained a dimer of the LXRβ-LBD in the asymmetric unit. The ligand was clearly defined in the initial Fo-Fc electron density map in both monomers. The binding pocket of the ligand is defined by residues PHE268, PHE271, THR272, LEU274, ALA275, SER278, ILE309, MET312, GLU315, THR316, ARG319, PHE329, LEU330, PHE340, LEU345, PHE349, ILE353, HIS435, GLN438, VAL439, LEU442, LEU449 and TRP457.

TABLE 1
Data collection and structure refinement statistics for 2-(4-{[2-(3-Chloro-phenyl)-5-methyl-
oxazol-4-ylmethyl]-ethyl-amino}-phenyl)-1,1,1,3,3,3-hexafluoropropan-2-ol co-crystal

| | |
|---|---|
| Data Collection | 2-(4-{[2-(3-Chloro-phenyl)-5-methyl-oxazol-4- |
| | ylmethyl]-ethyl-amino}-phenyl)-1,1,1,3,3,3- |
| | hexafluoro-propan-2-ol |
| Wavelength (Å) | 0.9511 |
| Resolution¹ (Å) | 20-3.2 (3.31-3.20) |
| Unique reflections¹ | 9790 |
| Completeness (%)¹ | 99.4 (99.7) |
| R_{merge} (%)^{1,2} | 8.3 (40.8) |
| <I/σI>¹ | 14.8 (3.4) |
| Unit Cell (Space group P4₃) | a = 58.0 Å, b = 58.0 Å, c = 181.8 Å, α = β = γ = 90° |
| Refinement | |
| Resolution (Å) | 20-3.2 (3.295-3.213) |
| R_{cryst}^{1,3} | 21.5 (29.2) |
| R_{free}^{1,4} | 28.5 (40.2) |
| R.m.s. deviations from | 0.010/1.249 |
| ideality Bond lengths (Å)/ | |
| angles (°) | |
| Main chain dihedral angles | 90.1/8.7/1.2/0.0 |
| (%) Most | |
| favoured/allowed/generous/ | |
| disallowed⁵ | |

¹Values in parentheses refer to the highest resolution bins.
²R_{merge} = Σ |I−<I>|/ΣI where I is the reflection intensity.
³R_{cryst} = Σ |Fₒ−<F_{c}>|/ΣFₒ where Fₒ is the observed and F_{c} is the calculated structure factor amplitude.
⁴R_{free} was calculated based on 5% of the total data omitted during refinement.
⁵Calculated with PROCHECK [Laskowski, R.A., MacArthur, M.W., Moss, D.S. & Thornton, J.M. PROCHECK: a program to check the stereochemical quality of protein structure. J. Appl. Crystallogr. 26, 283-291 (1993)].

### Example 2

### Crystal structure of human LXRβ-LBD with agonist 1,1,1,3,3,3-Hexafluoro-2-{2-methyl-1-[5-methyl-2-(3-trifluoromethyl-phenyl)-oxazol-4-ylmethyl]-1H-indo1-5-yl}-propan-2-ol

Protein used for crystallization of LXRβ-LBD together with 1,1,1,3,3,3-Hexafluoro-2-{2-methyl-1-[5-methyl-2-(3-trifluoromethyl-phenyl)-oxazol-4-ylmethyl]-1H-indol-5-yl}-propan-2-ol has been purified as described above. The protein was incubated with ligand in a 15-fold molar excess for 2 hours at room temperature. A short co-activator peptide (KDHQLLRYLLDKD) (Seq. Id. No. 3) from SRC-1 was added in 9-fold molar excess and incubation continued overnight at 4 ° C. Prior to crystallization experiments the protein was centrifuged at 20000 ×g. Crystallization droplets were set up at 22 ° C. by mixing 1.5 μl of protein solution with 0.5 μl reservoir solution in vapour diffusion hanging drop experiments. Crystals appeared out of 0.1 M Bis-Tris pH 5.5, 0.2 M MgCl₂, 25% PEG 3350 after 1 day and grew to final size of 0.2 mm×0.05 mm×0.05 mm within 3 days.

Crystals were harvested with paraffin oil as cryoprotectant and then flash frozen in a 100 K N₂ stream. Diffraction images were collected at a temperature of 100 K at the beamline X06SA of the Swiss Light Source and processed with the programs MOSFLM and SCALA yielding data to a resolution of 2.3 Å. Standard crystallographic programs from the CCP4 software suite were used to determine the structure by molecular replacement using an in-house LXRβ-LBD structure as search model. Refinement and model building cycles were performed with REFMAC and MOLOC, respectively (Table 2).

Results:

Crystals belong to space group P4₁2₁2 with cell axes a=92.7 Å, b=92.7 Å, c=273.2 Å, α=βγ=90°. The asymmetric unit contained a pair of dimers of the LXRβ-LBD. The ligand was clearly defined in the initial Fo-Fc electron density map in all monomers. The binding pocket of the ligand is formed by residues PHE268, PHE271, THR272, LEU274, ALA275, ILE277, SER278, GLU281, ILE309, MET312, LEU313, GLU315, THR316, ARG319, PHE329, LEU330, PHE340, LEU345, PHE349, ILE353, HIS435, GLN438, VAL439, LEU442, LEU449, LEU453, and TRP457.

TABLE 2
Data collection and structure refinement statistics for 1,1,1,3,3,3-Hexafluoro-2-{2-methyl-1-[5-
methyl-2-(3-trifluoromethyl-phenyl)-oxazol-4-ylmethyl]-1H-indol-5-yl}-propan-2-ol co-crystal

| | |
|---|---|
| Data Collection | 1,1,1,3,3,3-Hexafluoro-2-{2-methyl-1- |
| | [5-methyl-2-(3-trifluoromethyl-phenyl)- |
| | oxazol-4-ylmethyl]-1H-indol-5-yl}-propan-2-ol |
| Wavelength (Å) | 0.9789 |
| Resolution¹ (Å) | 30-2.3 (2.42-2.30) |
| Unique reflections¹ | 54660 |
| Completeness (%)¹ | 99.7 (99.9) |
| R_{merge} (%)^{1,2} | 7.7 (37.9) |
| <I/σI>¹ | 6.8 (2.0) |
| Unit Cell (Space group | a = 92.7 Å, = 92.7 Å, c = 273.2 Å, |
| P4₁2₁2) | α = β = γ = 90° |
| Refinement | |
| Resolution (Å) | 30-2.3 (2.359-2.30) |
| R_{cryst}^{1,3} | 21.4 (20.3) |
| R_{free}^{1,4} | 25.9 (28.5) |
| R.m.s. deviations from | 0.012/1.560 |
| ideality Bond lengths (Å)/ | |
| angles (°) | |
| Main chain dihedral angles | 93.0/6.0/0.6/0.4 |
| (%) Most | |
| favoured/allowed/generous/ | |
| disallowed⁵ | |

¹Values in parentheses refer to the highest resolution bins.
²R_{merge} = Σ |I−<I>|/ΣI where I is the reflection intensity.
³R_{cryst} = Σ |Fₒ−<F_{c}>|/ΣFₒ where Fₒ is the observed and F_{c} is the calculated structure factor amplitude.
⁴R_{free} was calculated based on 5% of the total data omitted during refinement.
⁵Calculated with PROCHECK [Laskowski, R.A., MacArthur, M.W., Moss, D.S. & Thornton, J.M. PROCHECK: a program to check the stereochemical quality of protein structure. J. Appl. Crystallogr. 26, 283-291 (1993)].

### Example 3

Crystal structure of human LXRβ-LBD with agonist (R,S)-Benzenesulfonyl-(6-chloro-2,3,4,9-tetrahydro-1H-carbazol-2-yl)-fluoro-acetic acid methyl ester

Protein used for crystallization of LXRβ-LBD together with - (R,S)-Benzenesulfonyl-(6chloro-2,3,4,9-tetrahydro-1H-carbazol-2-yl)-fluoro-acetic acid methyl ester has been purified as described above. The protein was incubated with ligand in a 15-fold molar excess for 2 hours at room temperature. A short co-activator peptide (ERHKILHRLLQEG) (Seq. Id. No. 4) from SRC-1 was added in 3-fold molar excess and incubation continued overnight at 4 ° C. Prior to crystallization experiments the protein was concentrated to 12 mg/ml and centrifuged at 20000 ×g. The crystallization droplet was set up at room temperature by mixing 1.5 μl of protein solution with 0.5 μl reservoir solution in vapour diffusion hanging drop experiments. Crystals appeared out of 0.1 M Bis-Tris pH 5.5, 0.2 M (NH⁴⁾²SO₄, 25% PEG 3350 after 1 day and grew to final size of 0.2 mm×0.1 mm×0.05 mm within 2 days.

Crystals were harvested with paraffin oil as cryoprotectant and then flash frozen in a 100 K N₂ stream. Diffraction images were collected at a temperature of 100 K at the beamline X06SA of the Swiss Light Source and processed with the programs DENZO and SCALEPACK yielding data to a resolution of 2.3 Å. Standard crystallographic programs from the CCP4 software suite were used to determine the structure by molecular replacement using an in-house LXR-LBD structure as search model. Refinement and model building cycles were performed with REFMAC and MOLOC, respectively (Table 3).

Results:

Crystals belong to space group P1 with cell axes a=47.5 Å, b=112.2 Å, c=114.1 Å, α=74.0°, β=98.4°, γ=79.6°. The asymmetric unit is formed by three dimers of LXRβ-LBD. Every monomer binds two ligand molecules which are all clearly defined in the Fo-Fc electron density map. The ligand located close to helix 10/11 and helix 12 shows interactions with residues

PHE268, PHE271, THR272, ALA275, ILE209, MET312, LEU313, THR316, PHE340, LEU345, PHE349, ILE353, HIS435, VAL439, LEU442, LYS447, LYS448, LEU449, PRO450, LEU453, and TRP457 . The binding pocket for the ligand beneath helix1 is formed by residues GLN235, CYS238, ASN239, PHE243, PHE271, LEU274, ALA275, SER278, GLU281, ILE282, PHE285, ILE311, MET312, GLU315, THR316, ARG319, ILE327, THR328, PHE329, TYR335, PHE340, ILE374.

TABLE 3
Data collection and structure refinement statistics for (R,S)-Benzenesulfonyl-(6-chloro-2,3,4,9-tetrahydro-
1H-carbazol-2-yl)-fluoro-acetic acid methyl ester co-crystal

| | |
|---|---|
| Data Collection | (R,S)-Benzenesulfonyl-(6-chloro-2,3,4,9-tetrahydro- |
| | 1H-carbazol-2-yl)-fluoro-acetic acid methyl ester |
| Wavelength (Å) | 1.0000 |
| Resolution¹ (Å) | 20-2.3 (2.38-2.30) |
| Unique reflections¹ | 95265 |
| Completeness (%)¹ | 94.8 (88.0) |
| R_{merge} (%)^{1,2} | 4.6 (30.0) |
| <I/σI>¹ | 15.4 (3.5) |
| Unit Cell (Space group P4₃) | a = 47.5 Å, b = 112.2 Å, c = 114.1 Å, |
| | α = 74.0°, β = 98.4°, γ = 79.6° |
| Refinement | |
| Resolution (Å) | 20-2.3 (2.36-2.30) |
| R_{cryst}^{1,3} | 19.0 (22.2) |
| R_{free}^{1,4} | 26.3 (28.3) |
| R.m.s. deviations from | 0.009/1.461 |
| ideality Bond lengths (Å)/ | |
| angles (°) | |
| Main chain dihedral angles | 92.1/7.1/0.8/0.4 |
| (%) Most | |
| favoured/allowed/generous/ | |
| disallowed⁵ | |

¹Values in parentheses refer to the highest resolution bins.
²R_{merge} = Σ |I−<I>|/ΣI where I is the reflection intensity.
³R_{cryst} = Σ |Fₒ−<F_{c}>|/ΣFₒ where Fₒ is the observed and F_{c} is the calculated structure factor amplitude.
⁴R_{free} was calculated based on 5% of the total data omitted during refinement.
⁵Calculated with PROCHECK [Laskowski, R.A., MacArthur, M.W., Moss, D.S. & Thornton, J.M. PROCHECK: a program to check the stereochemical quality of protein structure. J. Appl. Crystallogr. 26, 283-291 (1993)].

1. A co-crystal of a liver X receptor beta Ligand binding domain (LXRβ-LBD) polypeptide with a ligand, wherein the crystal belongs to space group P4₃, and wherein further the LXRβ-LBD polypeptide V comprises a sequence having a sequence similarity to the ligand binding domain of a polypeptide of SEQ ID NO:1 of at least 80%.

2. The crystal of claim 1, wherein the crystal has unit cell dimensions of a=b =58±4 Å, c=181±4 Å, α=β=γ=90°±3°, preferably ±2°, more preferably ±1°, most preferably α=β=γ=90°.

3. The co-crystal of claim 1, wherein the ligand is 2-(4-{[2-(3-Chloro=phenyl)-5-methyl-oxazol-4-ylmethyl]-ethyl-amino}-phenyl)-1,1,1,3,3,3-hexafluoro-propan-2-ol

4. A co-crystal of a liver X receptor beta Ligand binding domain (LXRβ-LBD) with a Ligand, wherein the crystal belongs to space group P4₁2₁2, and wherein further the LXRβ-LBD polypeptide V comprises a sequence having a sequence similarity to the ligand binding domain of a polypeptide of SEQ ID NO:1 of at least 80%.

5. The crystal of claim 4, wherein the crystal has unit cell dimensions of a=b =92 ±4 Å, c=273±4 Å, α=β=γ=90°±3°, preferably ±2°, more preferably ±1°, most preferably α=β=γ=90°.

6. The co-crystal of claim 4, wherein the Ligand is 1,1,1,3,3,3-Hexafluro-2-{2-methyl-1-[5-methyl-2-(3-trifluoromethyl-phenyl)-oxazol-4-ylmethyl]-1 H-indol -5-yl}propan-2-ol.

7. A co-crystal of a liver X re captor beta Ligand binding domain (LXRβ-LBD) with a Ligand, wherein the crystal belongs to space group P1, and wherein further the LXRβ-LBD polypeptide V comprises a sequence having a sequence similarity to the ligand binding domain of a polypeptide of SEQ ID NO:1 of at least 80%.

8. The crystal of claim 7, wherein the crystal has unit cell dimension of a=47±4 Å, b=98±4 Å, c=114±4 Å, α=74°±3°, β=98°±3°, γ=79°±3°, preferably ±2°, more preferably ±1°, most preferably α=74°, β=98°, γ=79°.

9. The co-crystal of claim 7, wherein the Ligand is (R,S)-Benzenesulfonyl-(6-chloro-2,3,4,9-tetrahydro-1H-carbazol-21-yl)-fluoro-acetic acid methyl ester.

10. The co-crystal of claim 1, wherein the LXRβ-LBD polypeptide is a polypeptide comprising a sequence having a similarity to the Ligand binding domain of a polypeptide of Seq. Id. No. 1 of at least 85%, more preferably at least 90%, even more preferably at least 95%, most preferably 100%.

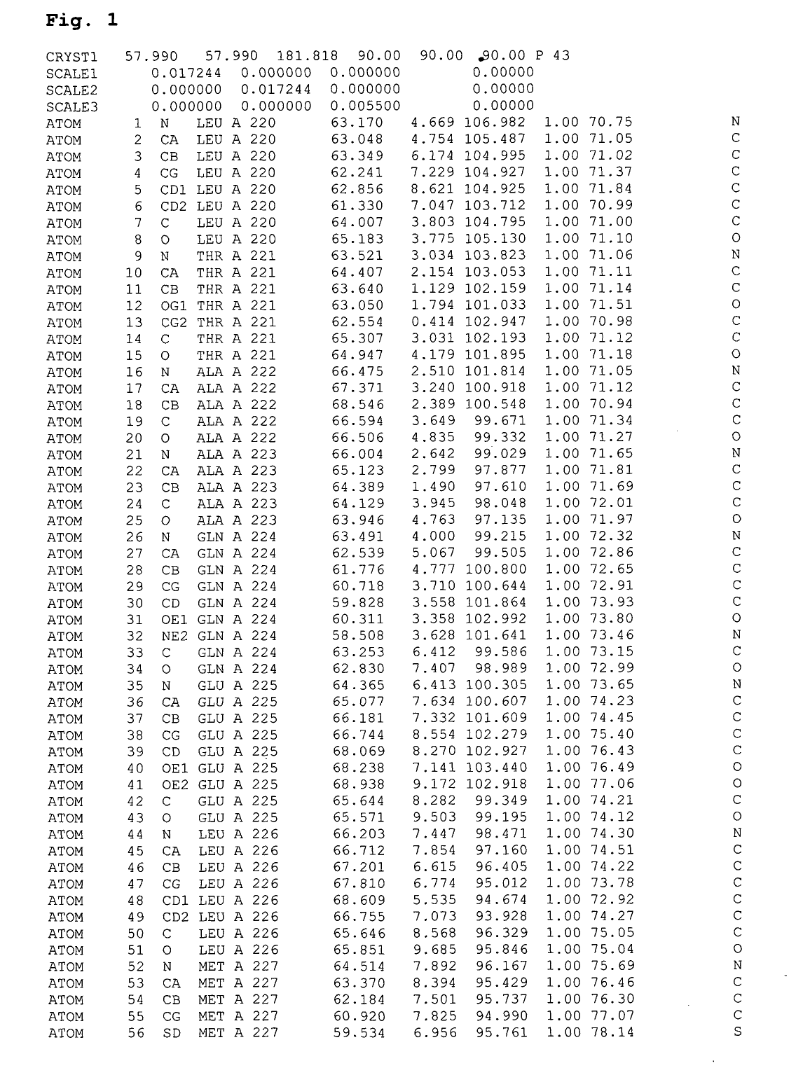

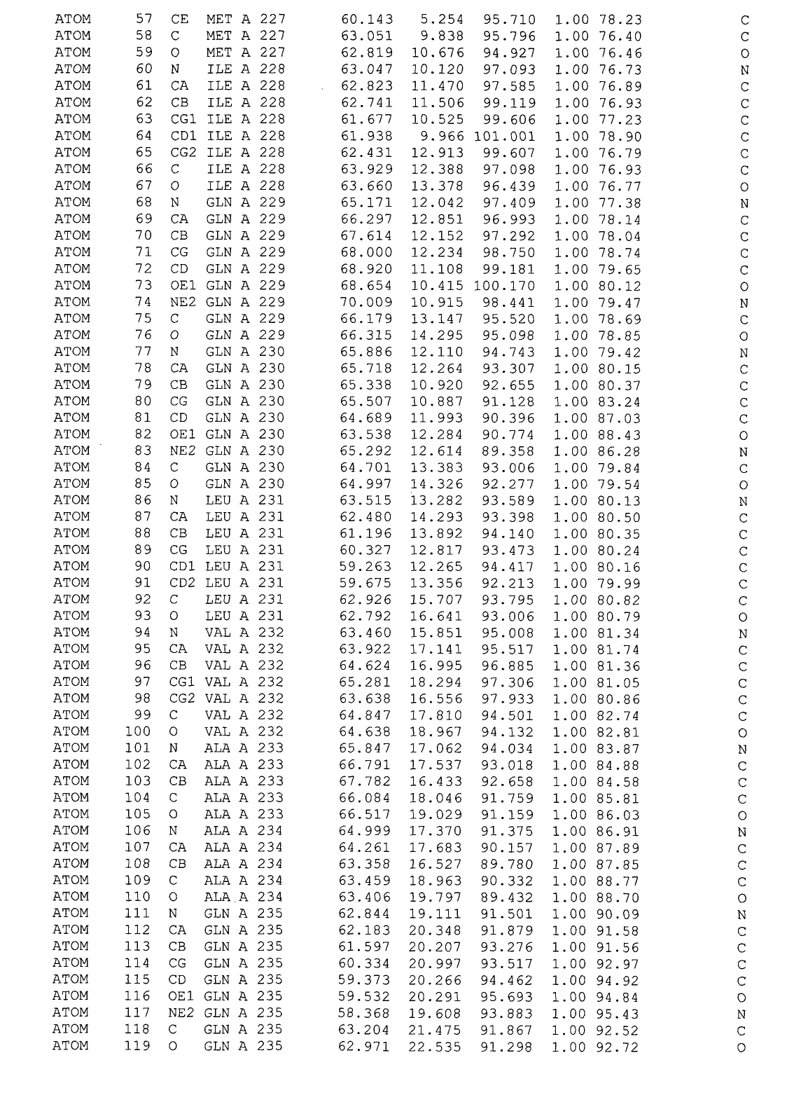

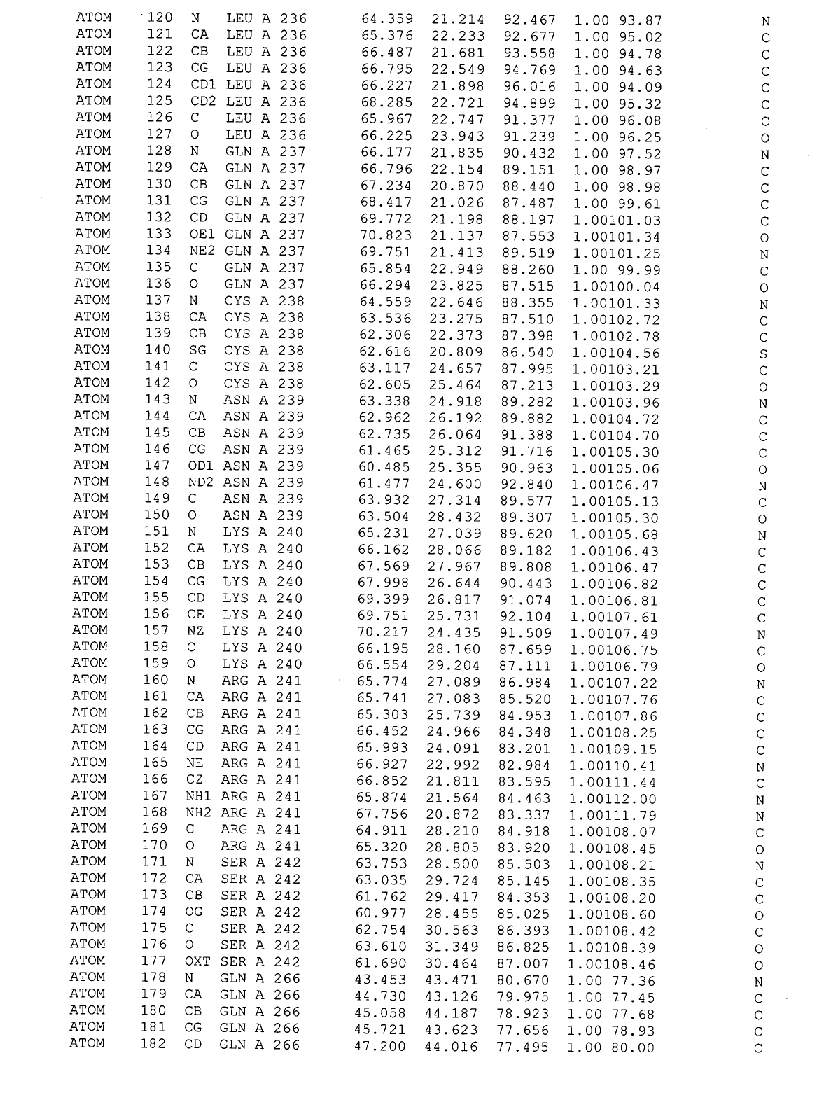

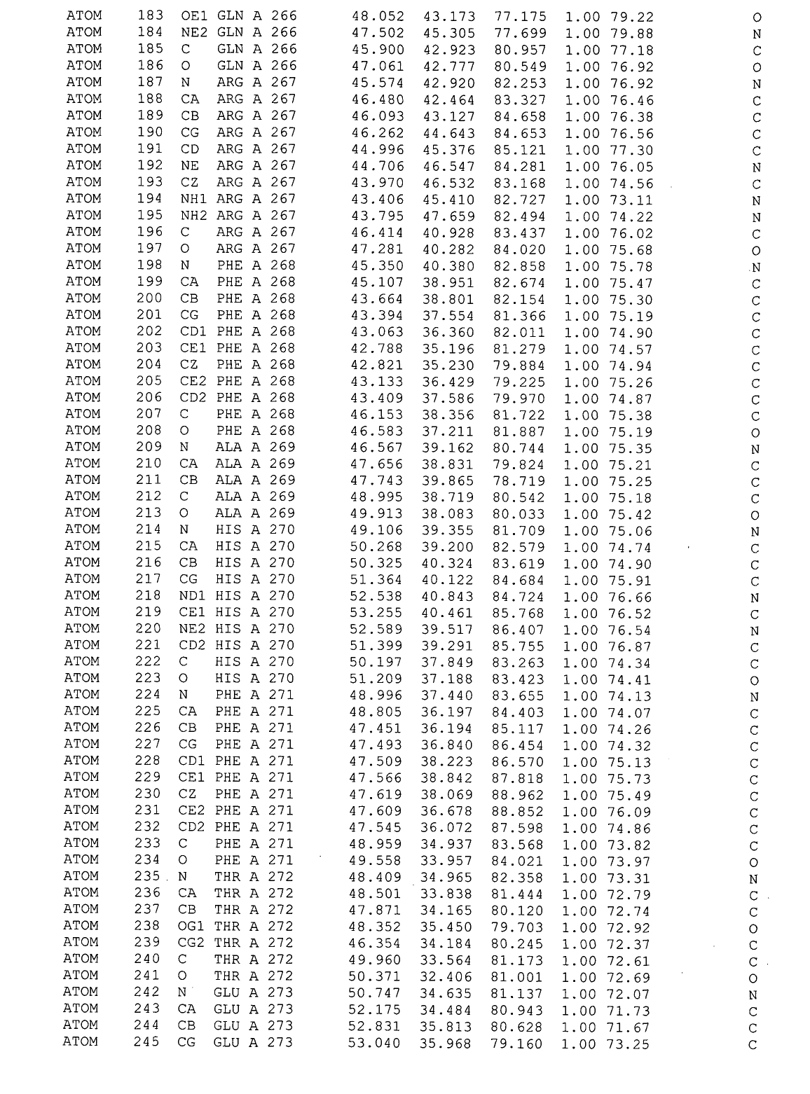

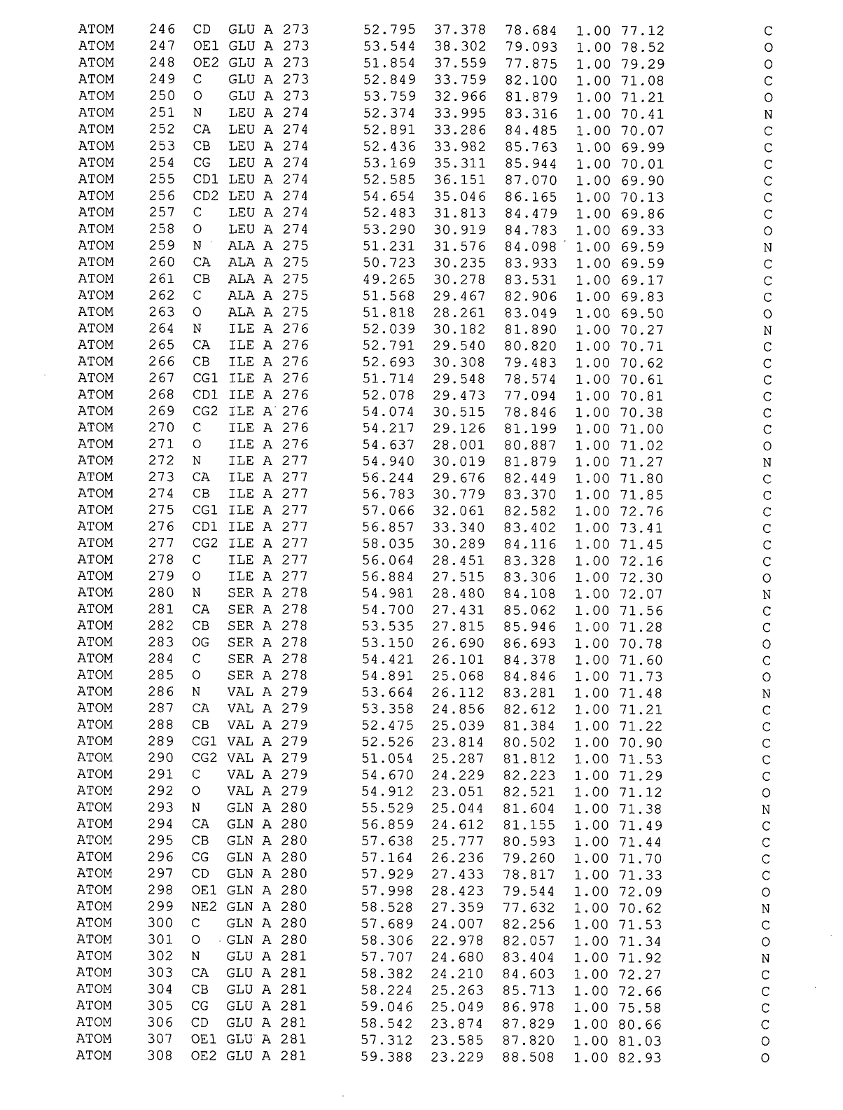

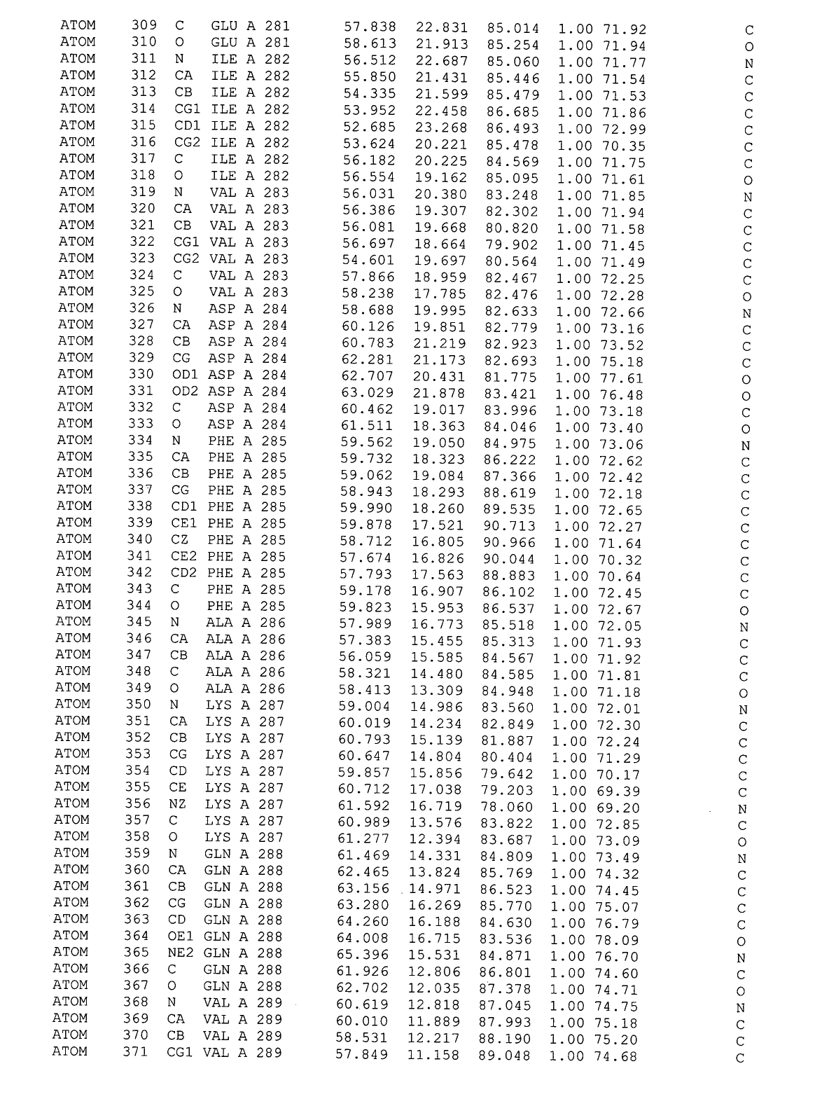

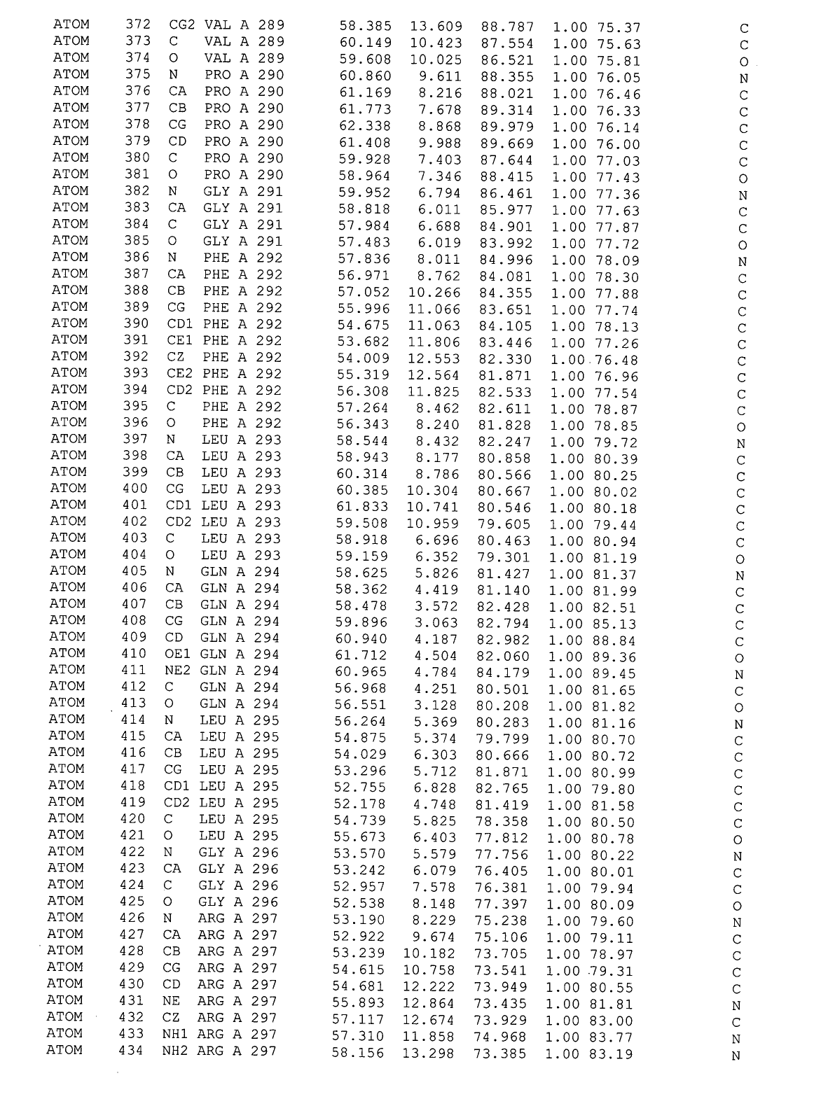

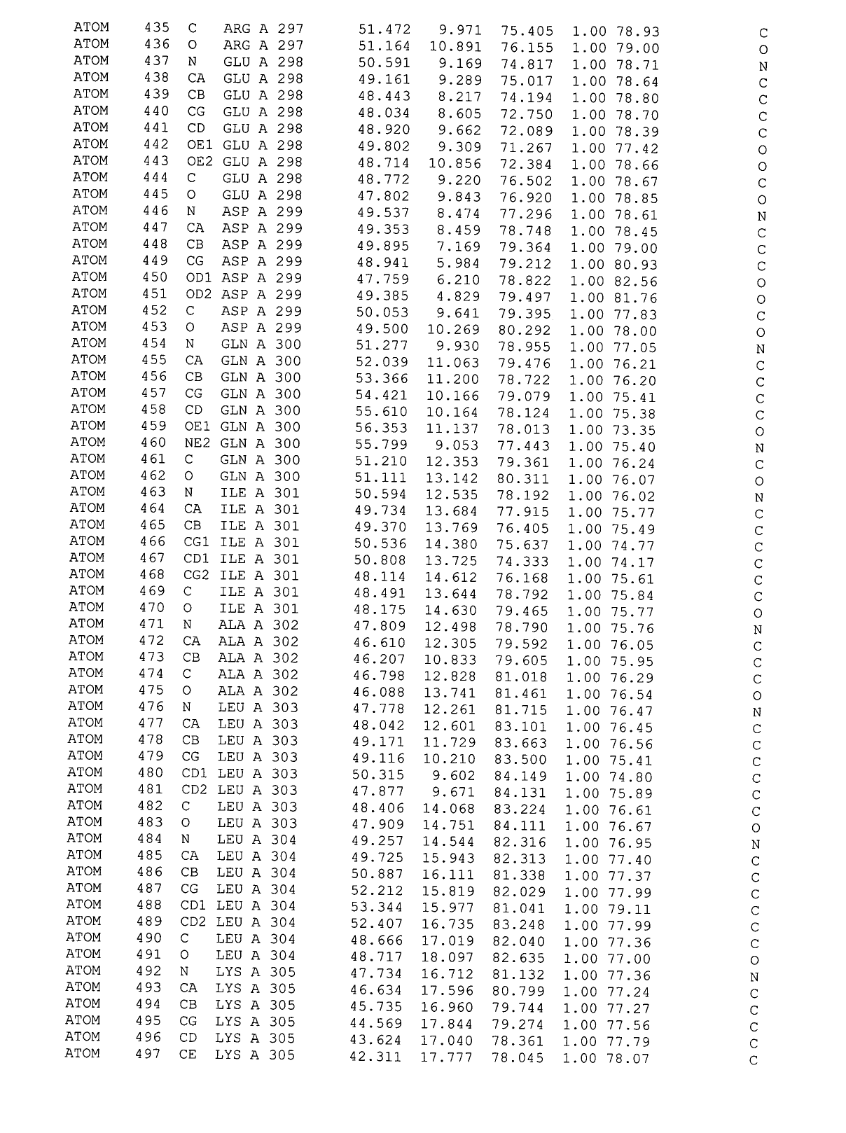

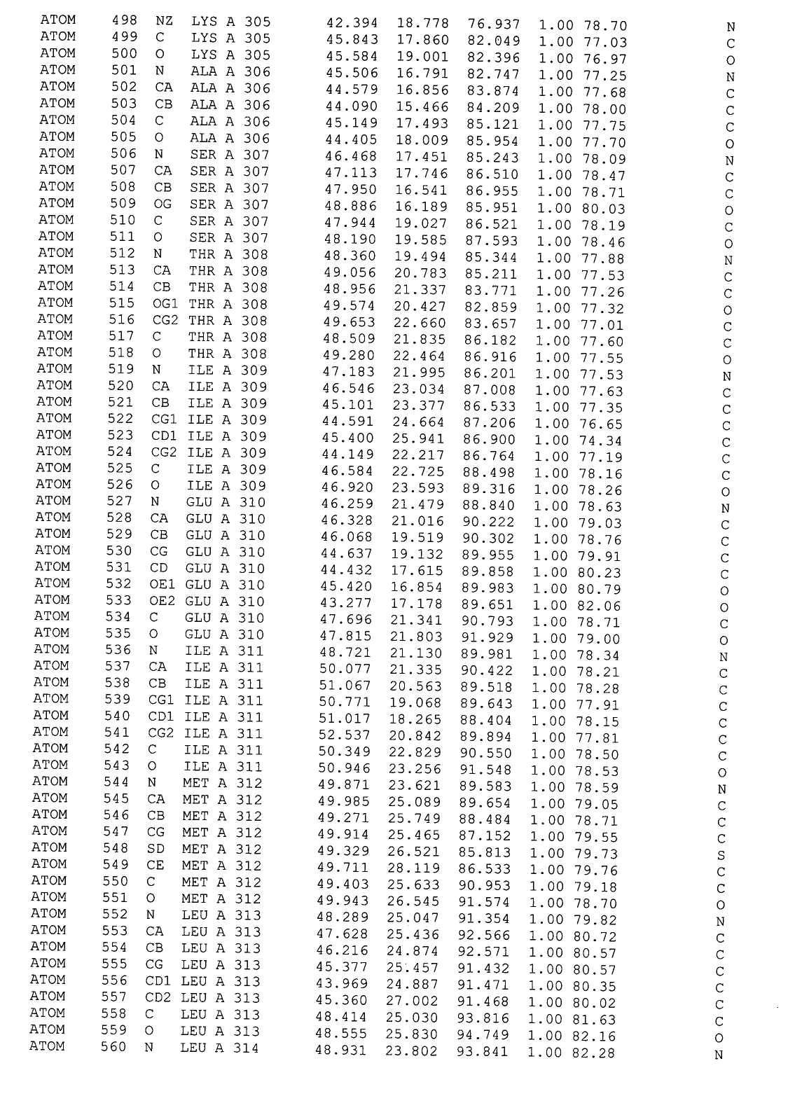

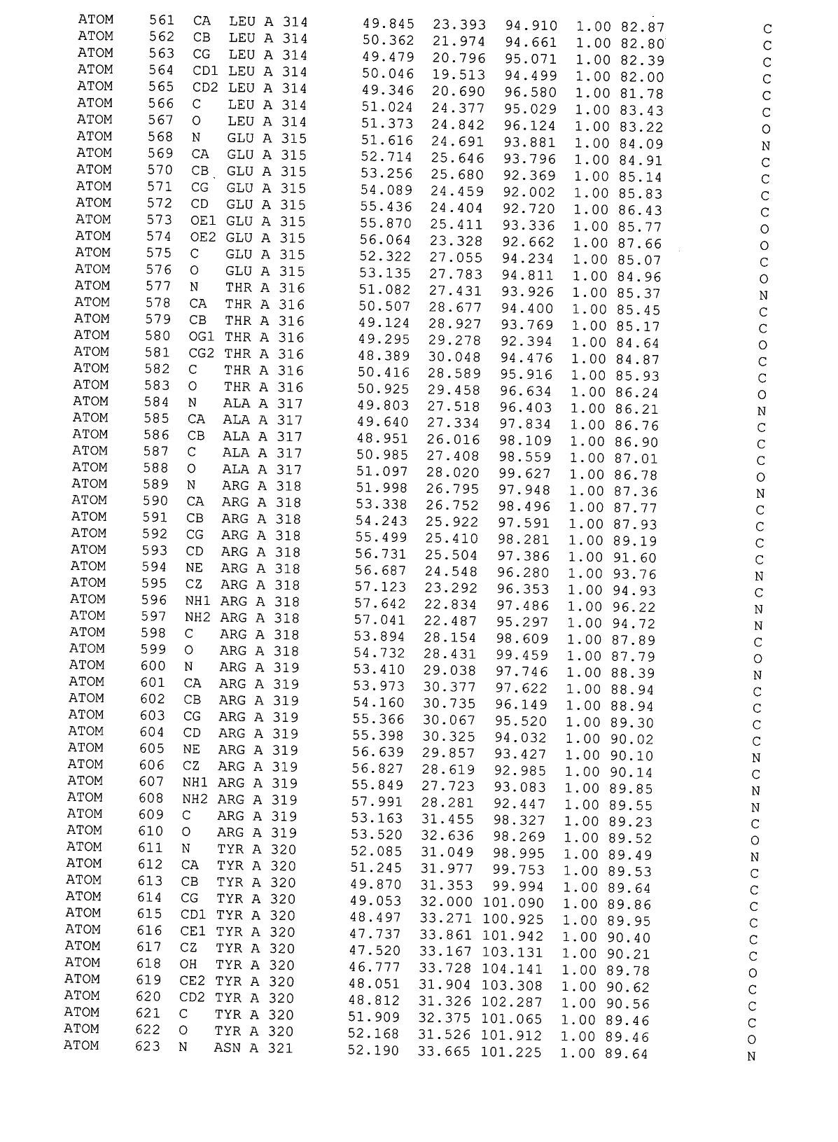

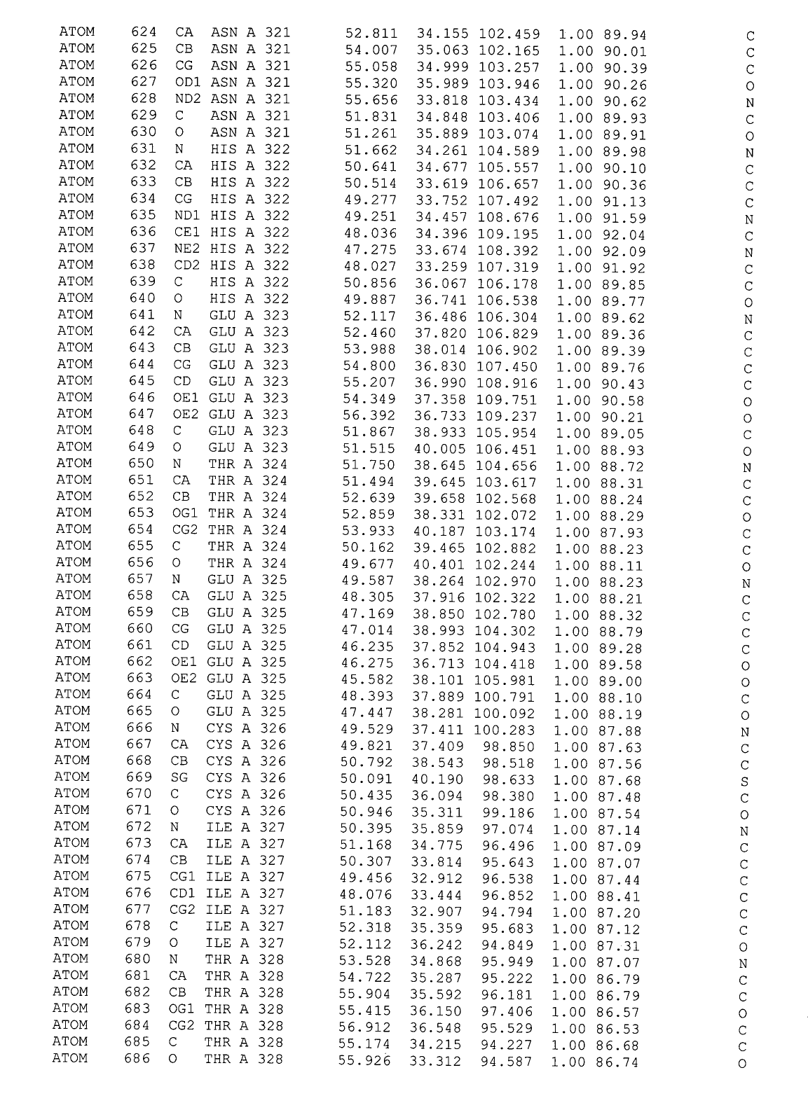

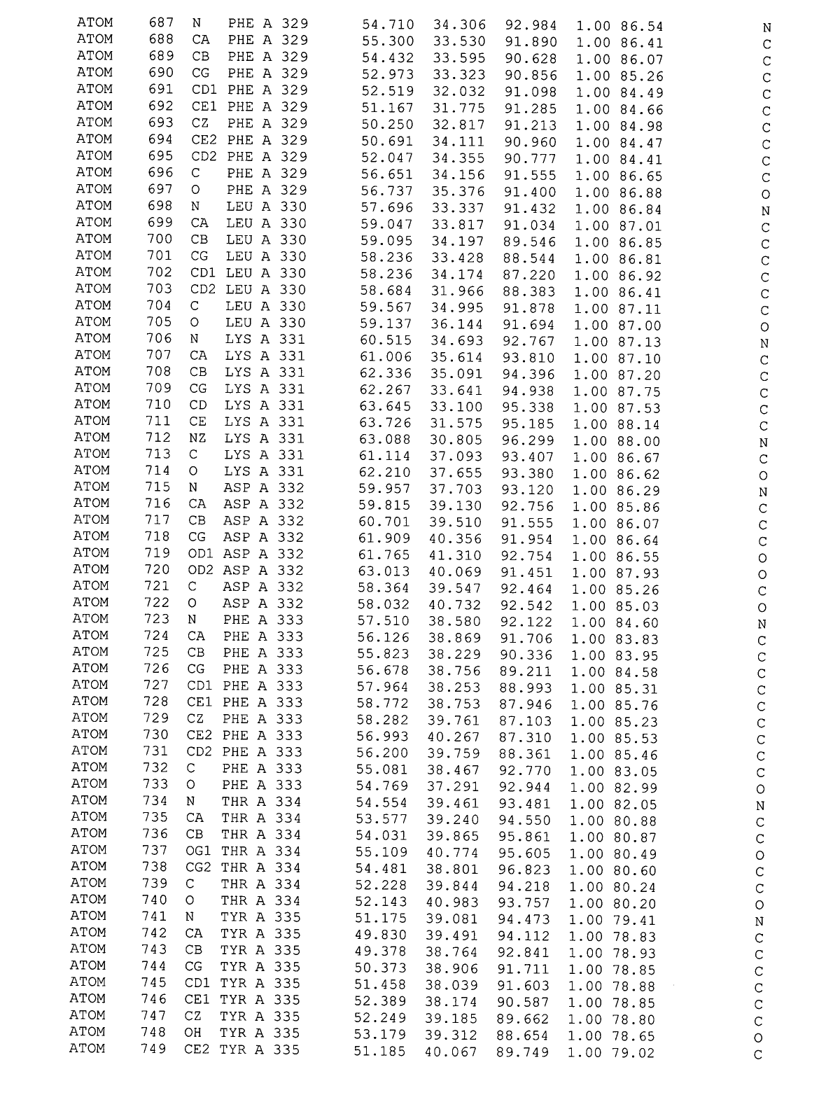

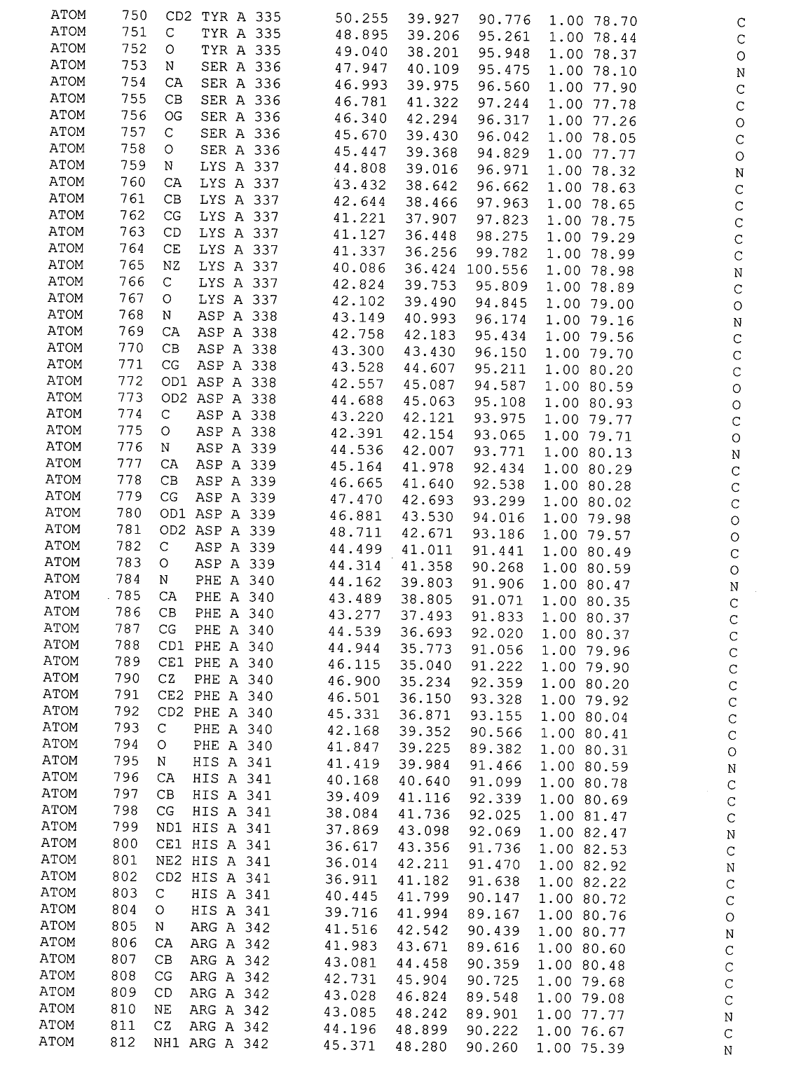

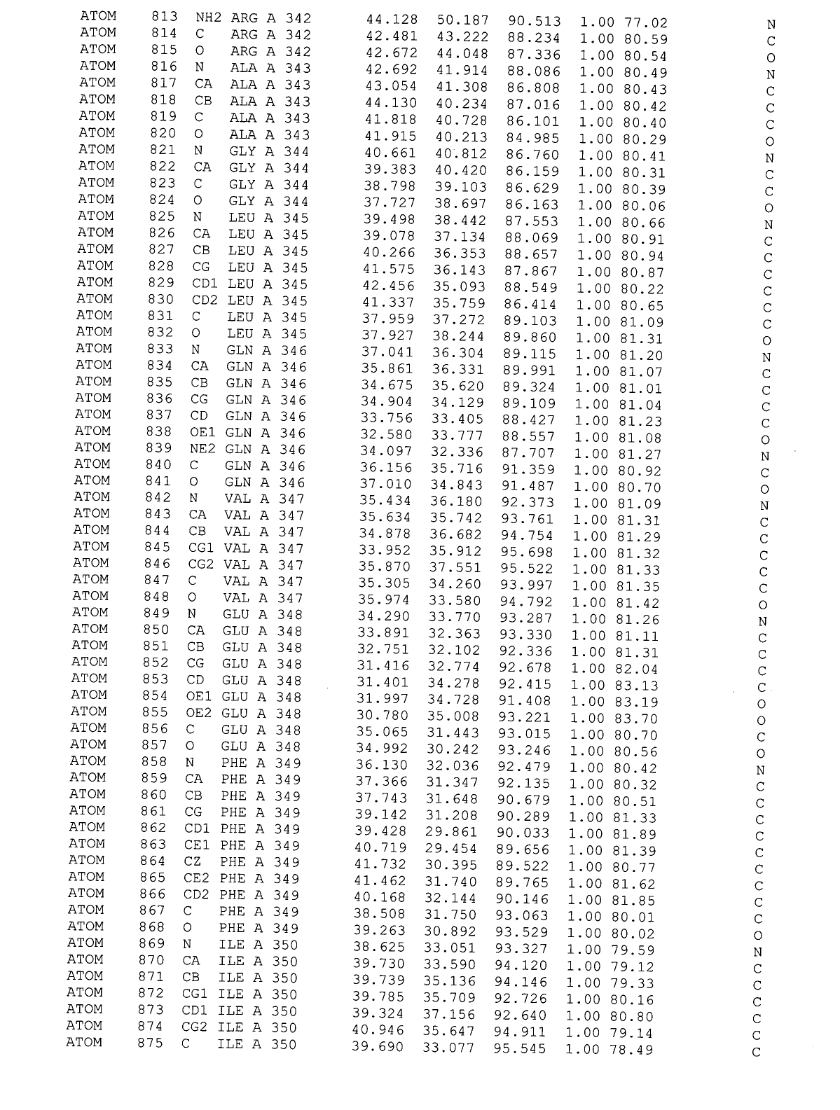

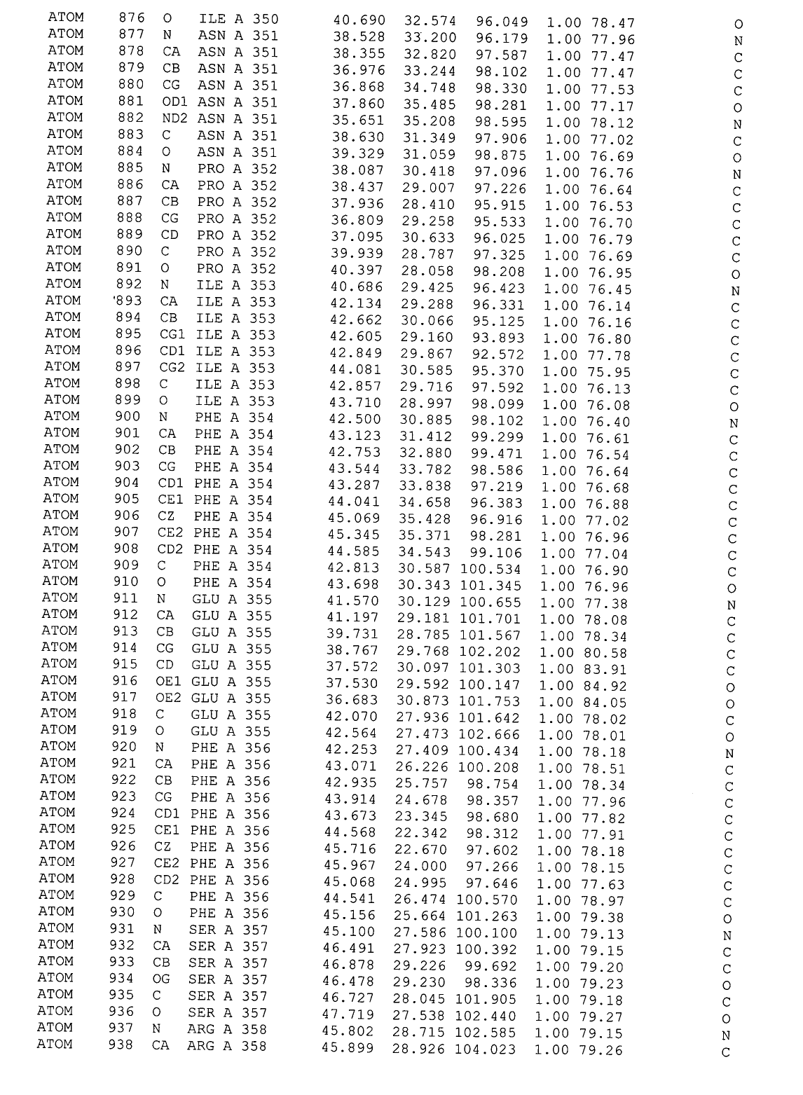

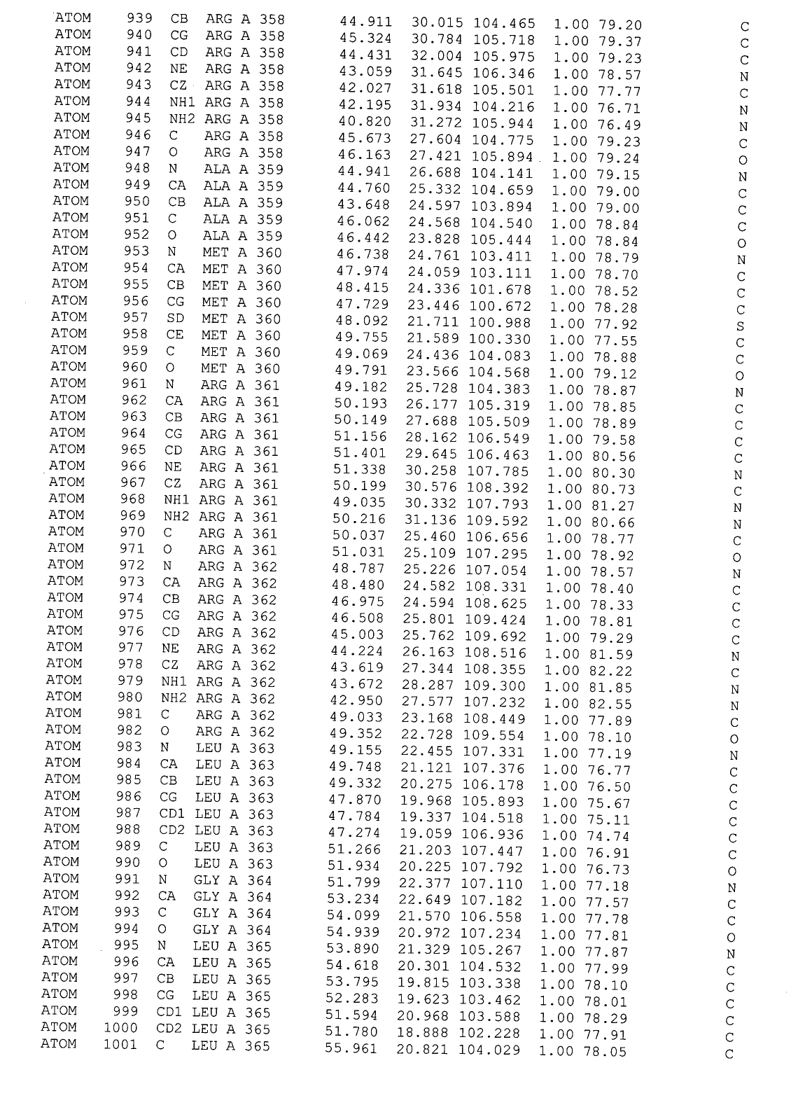

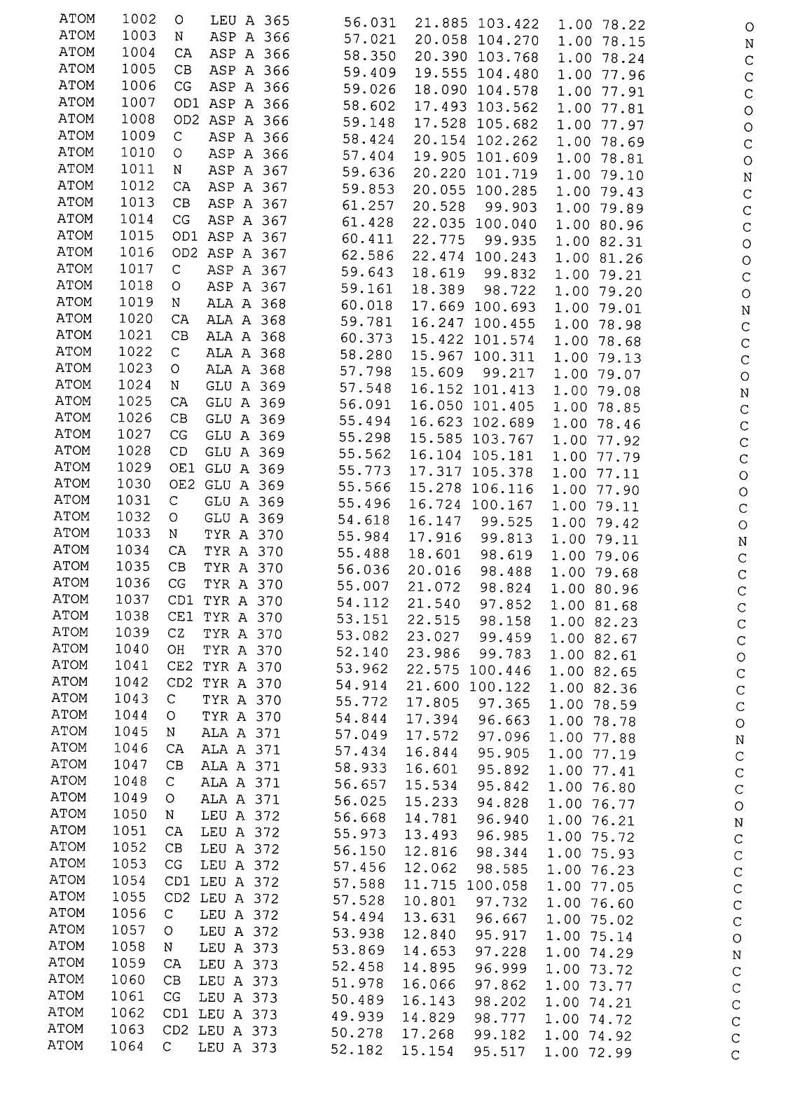

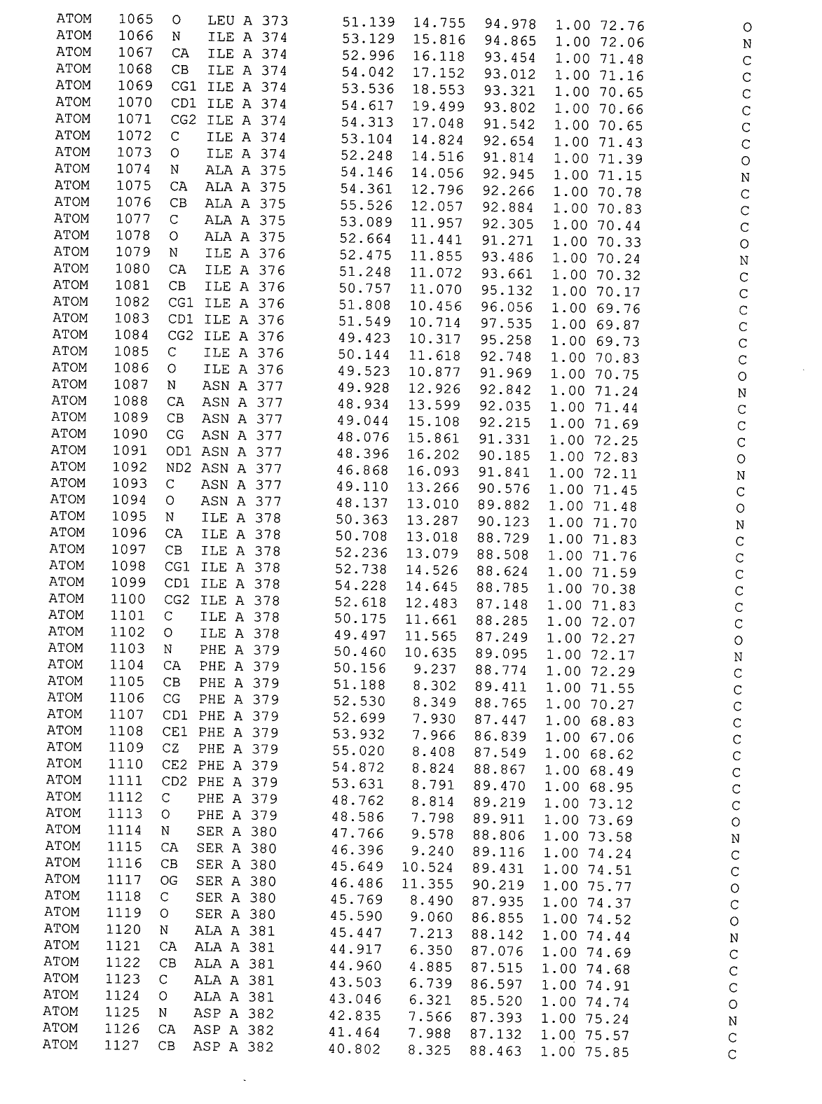

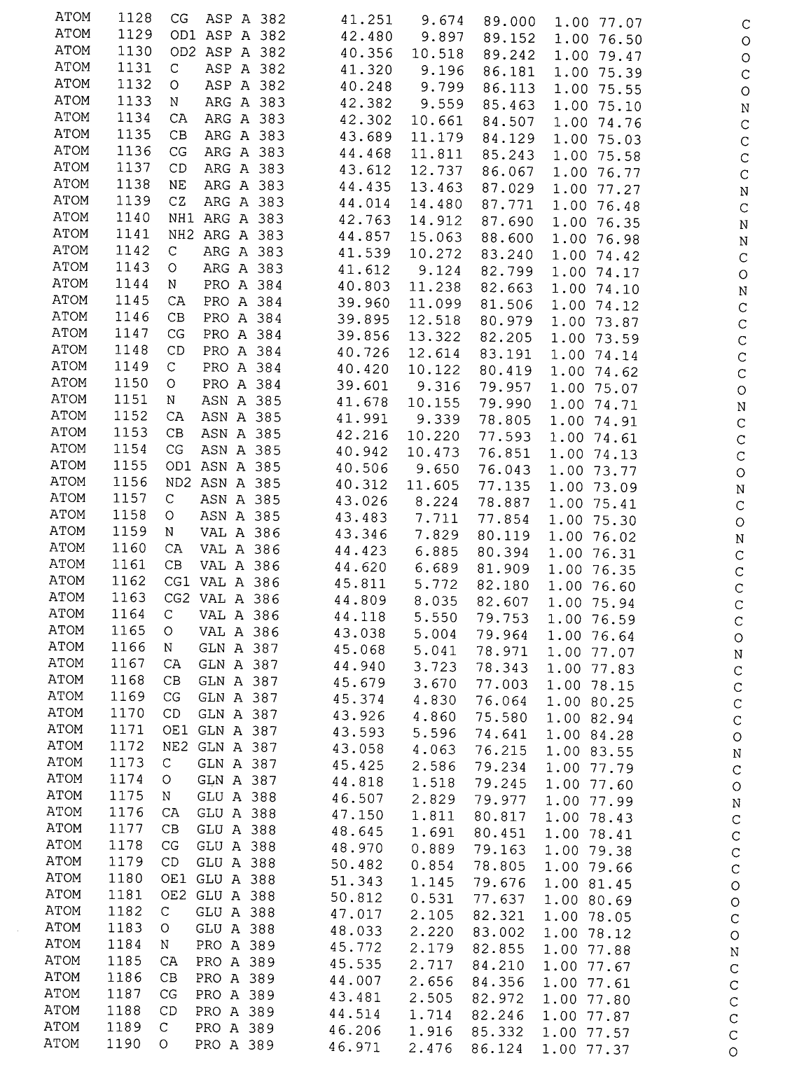

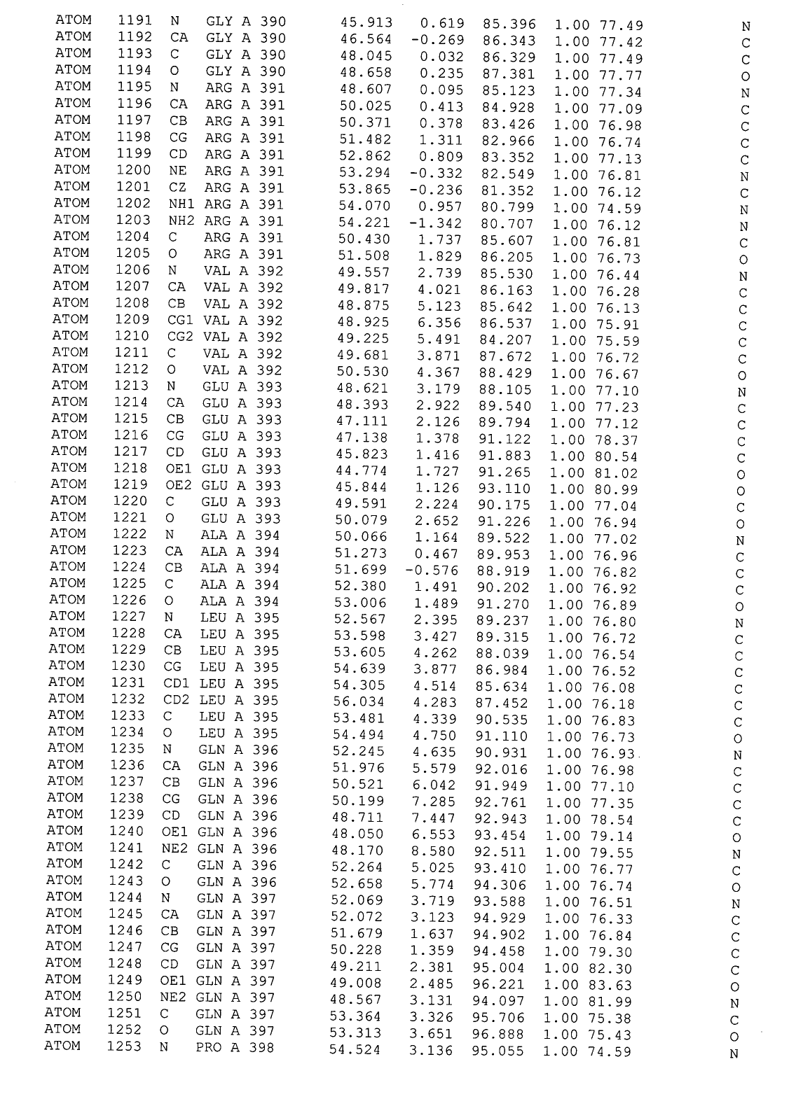

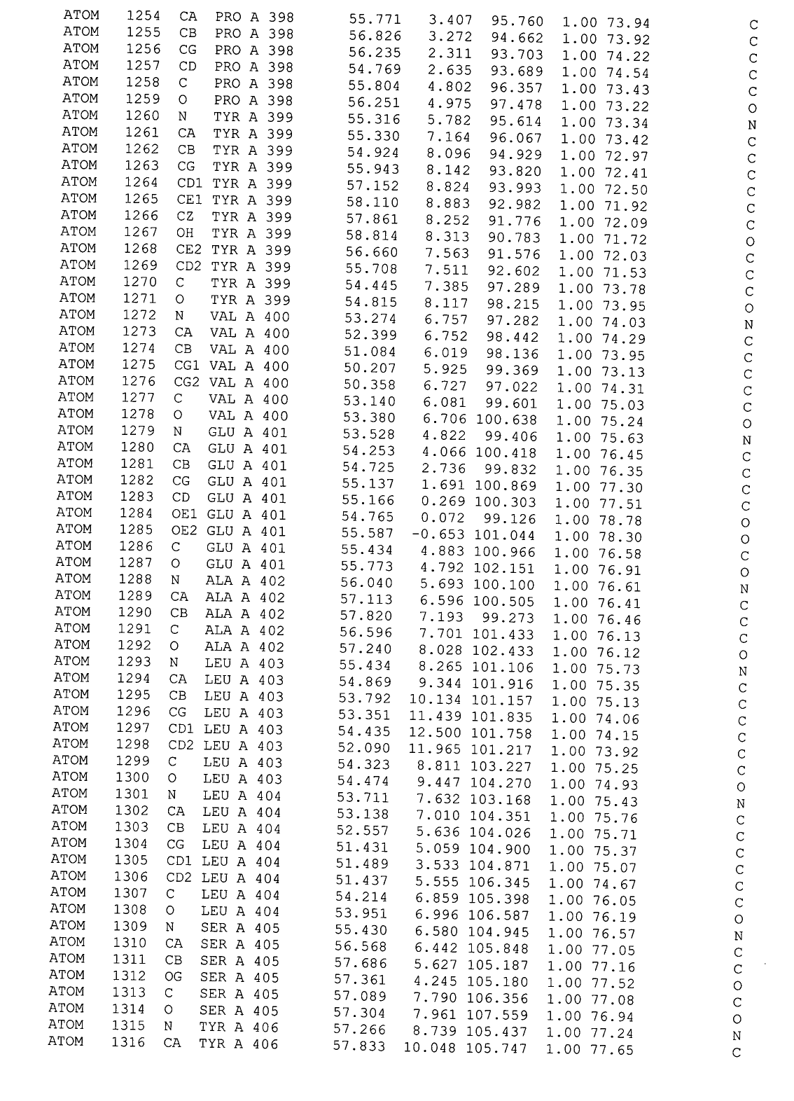

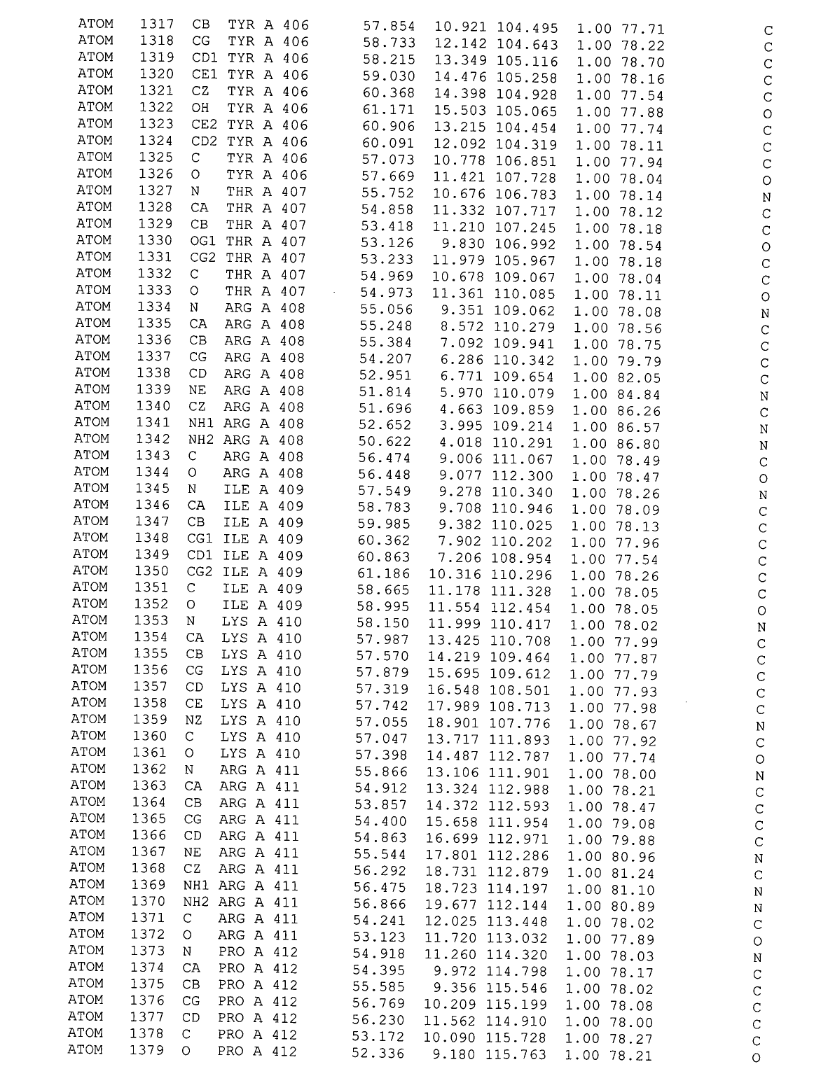

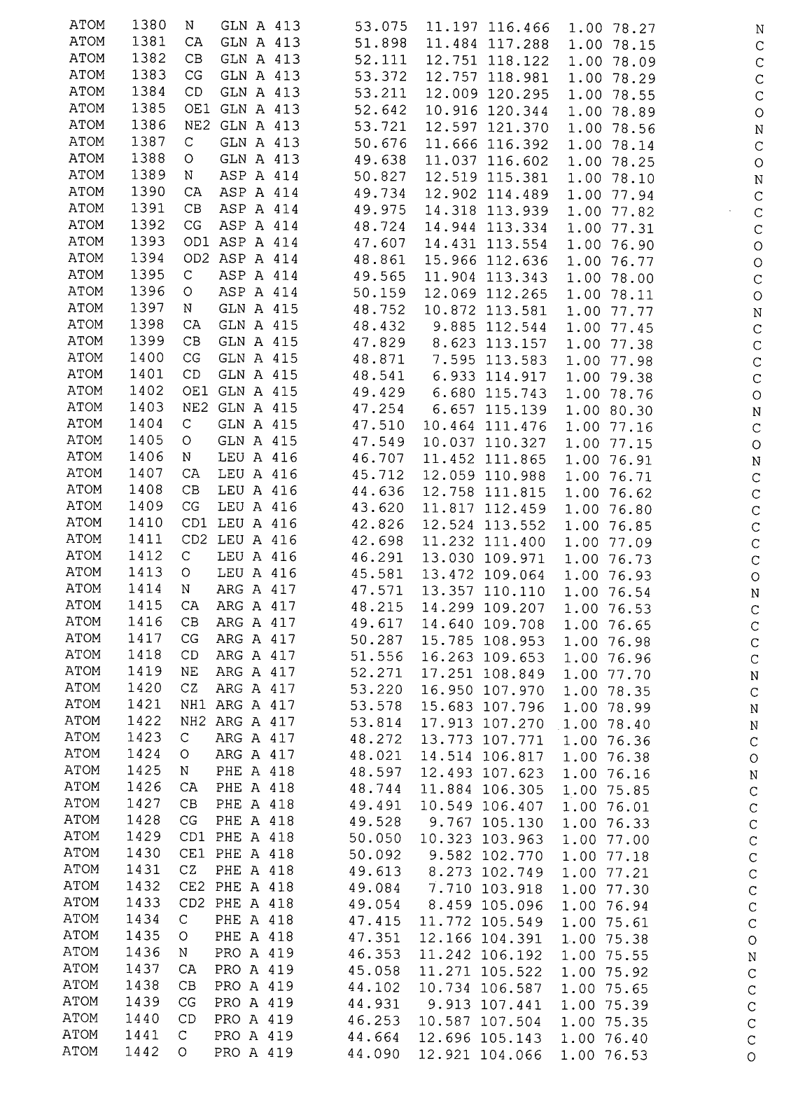

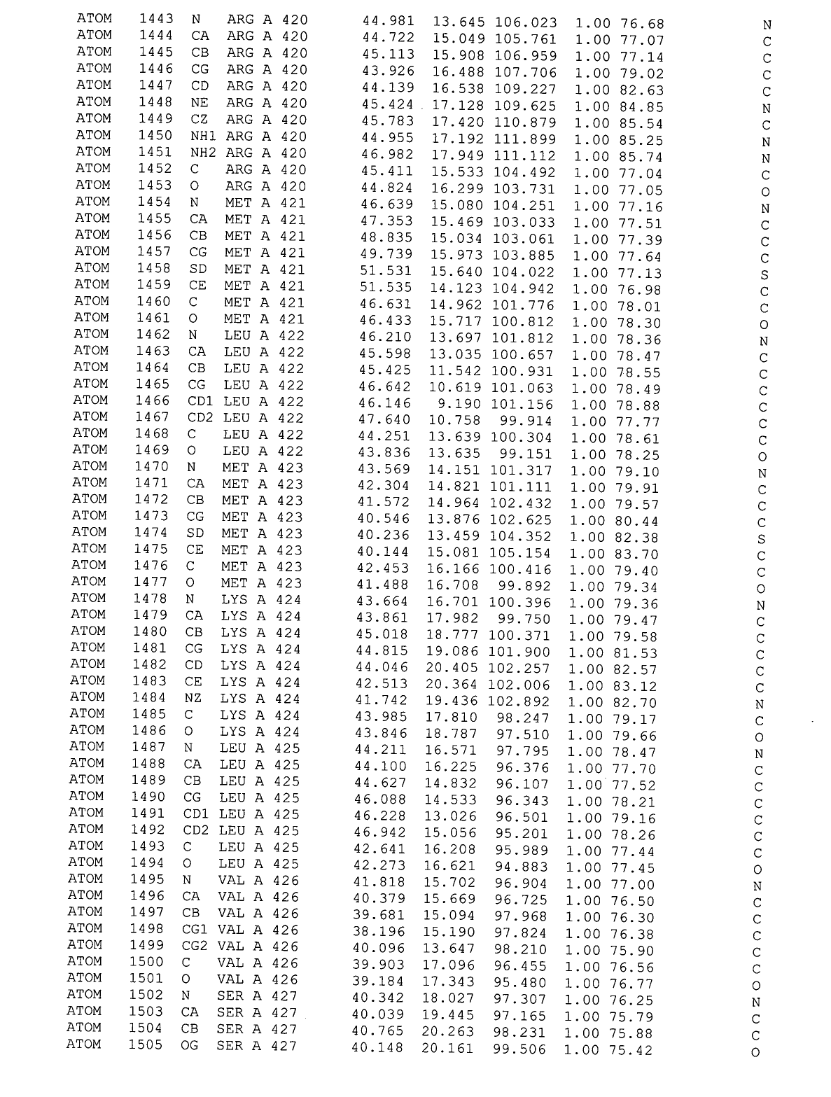

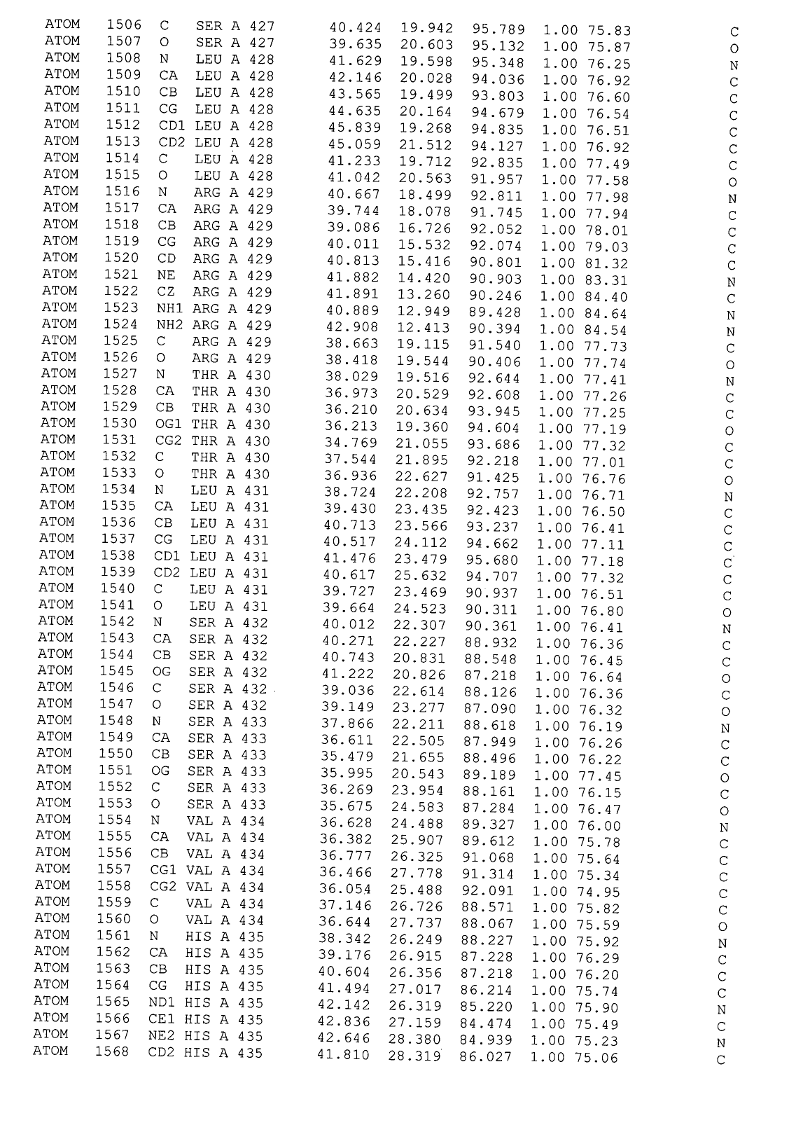

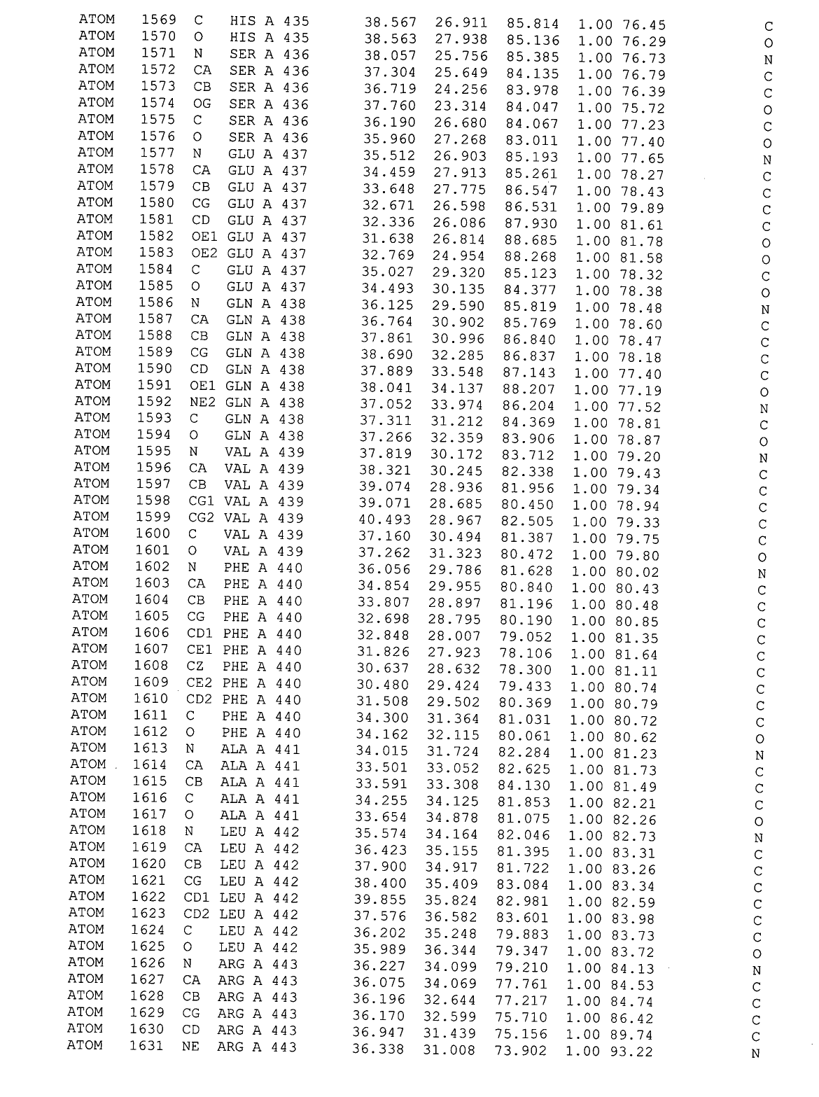

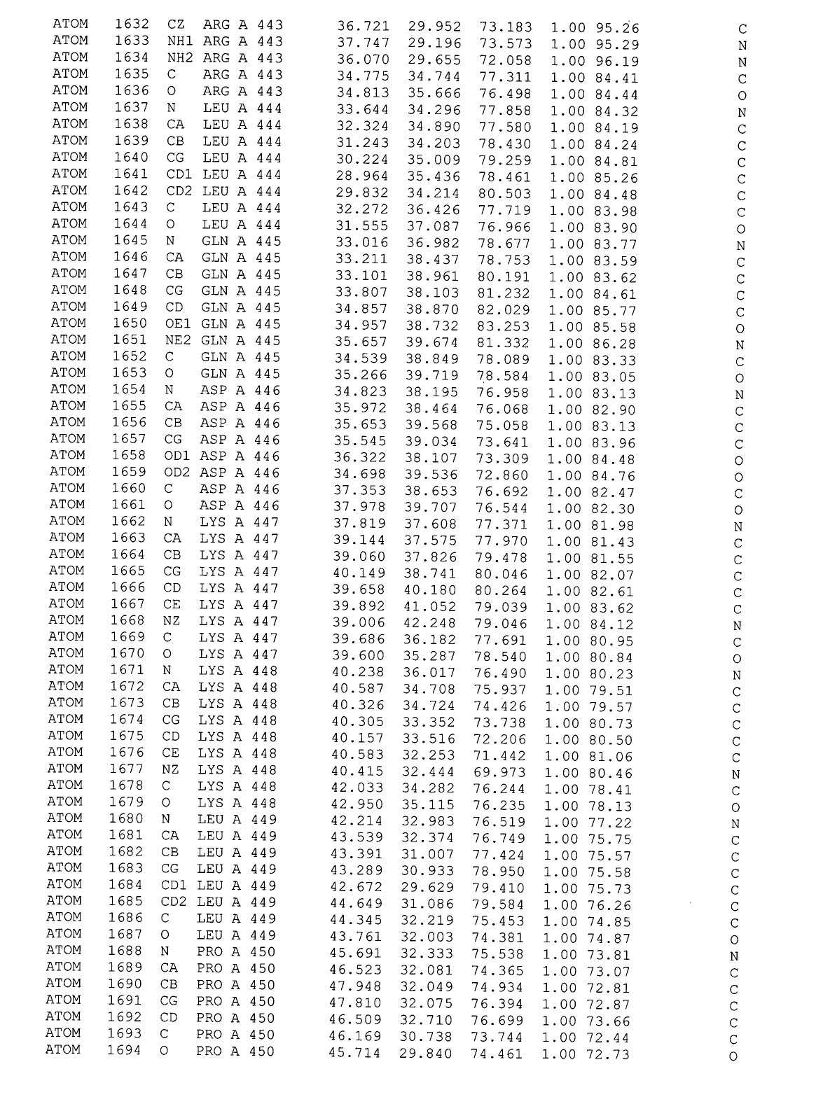

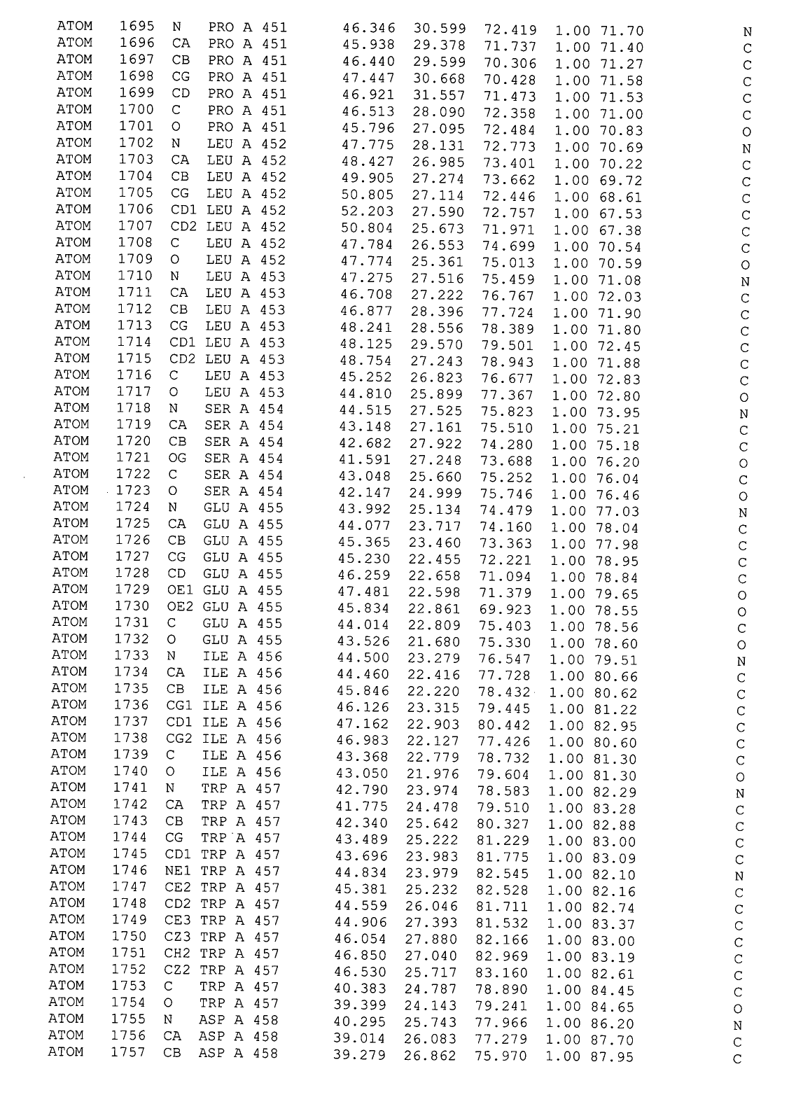

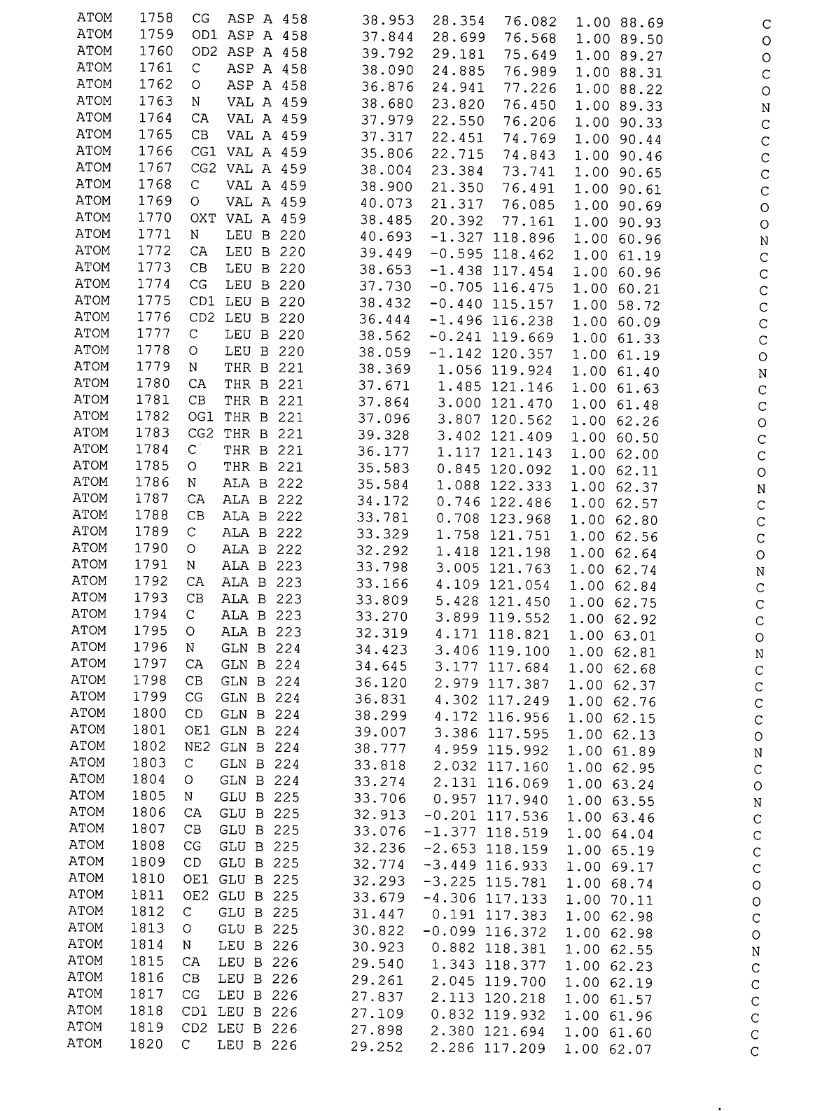

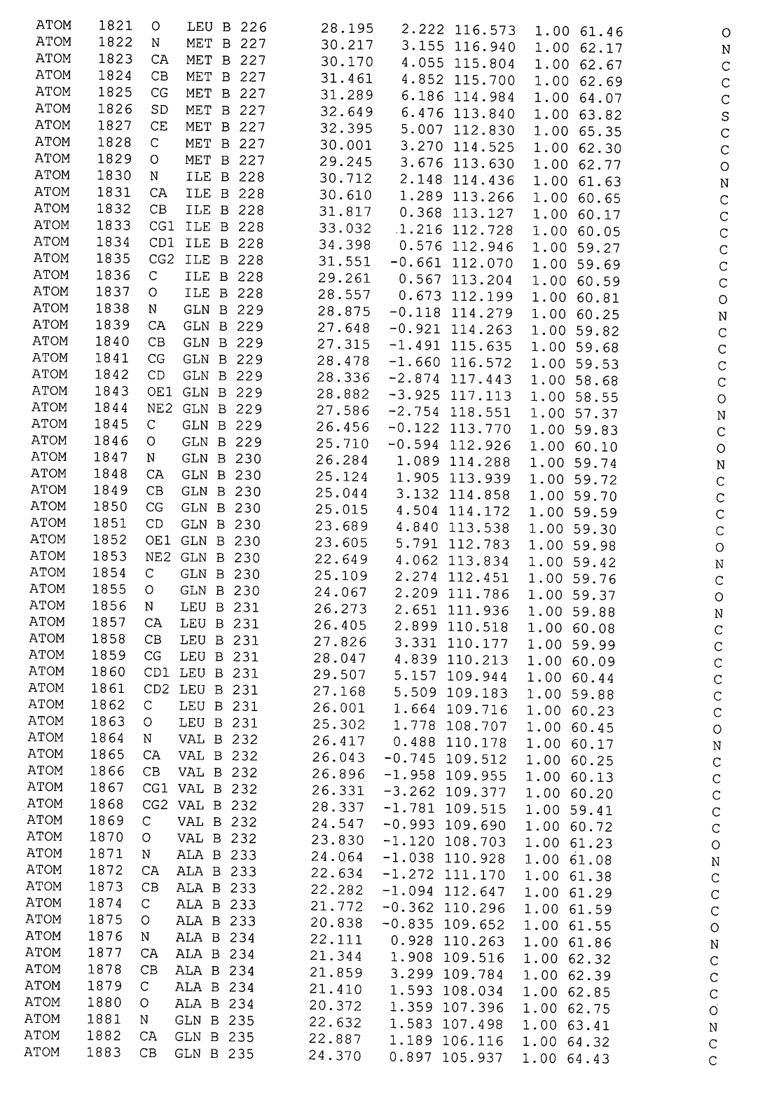

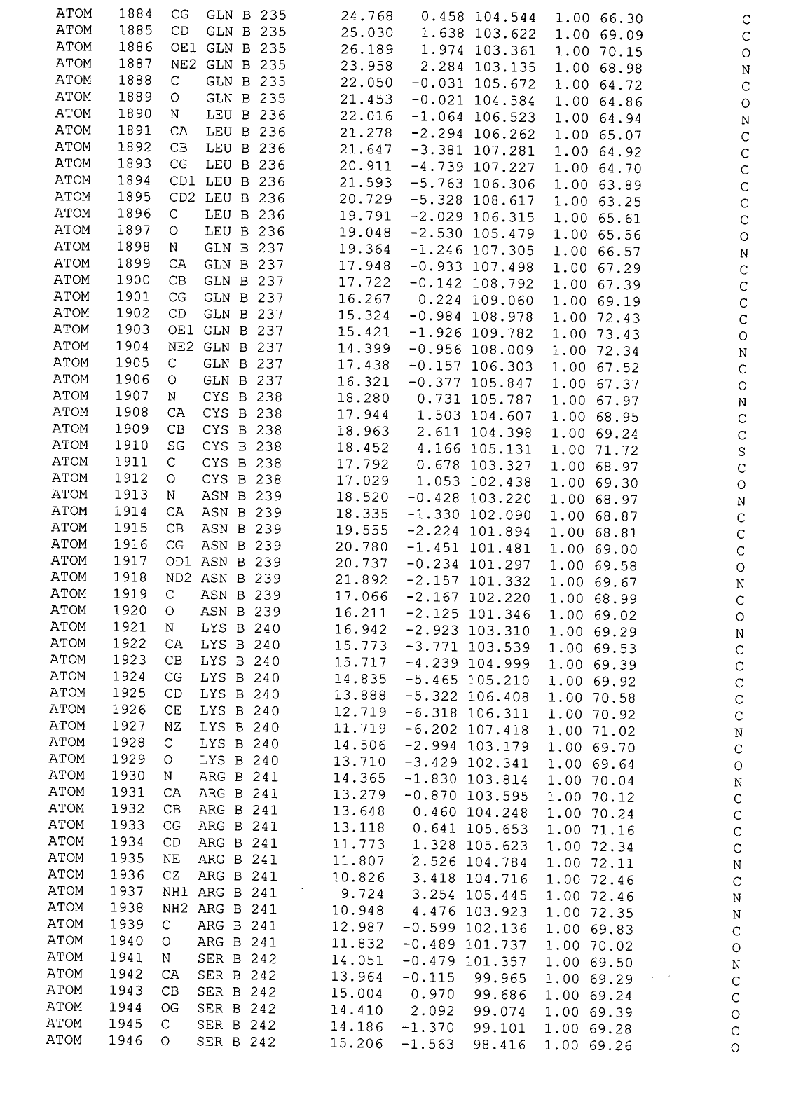

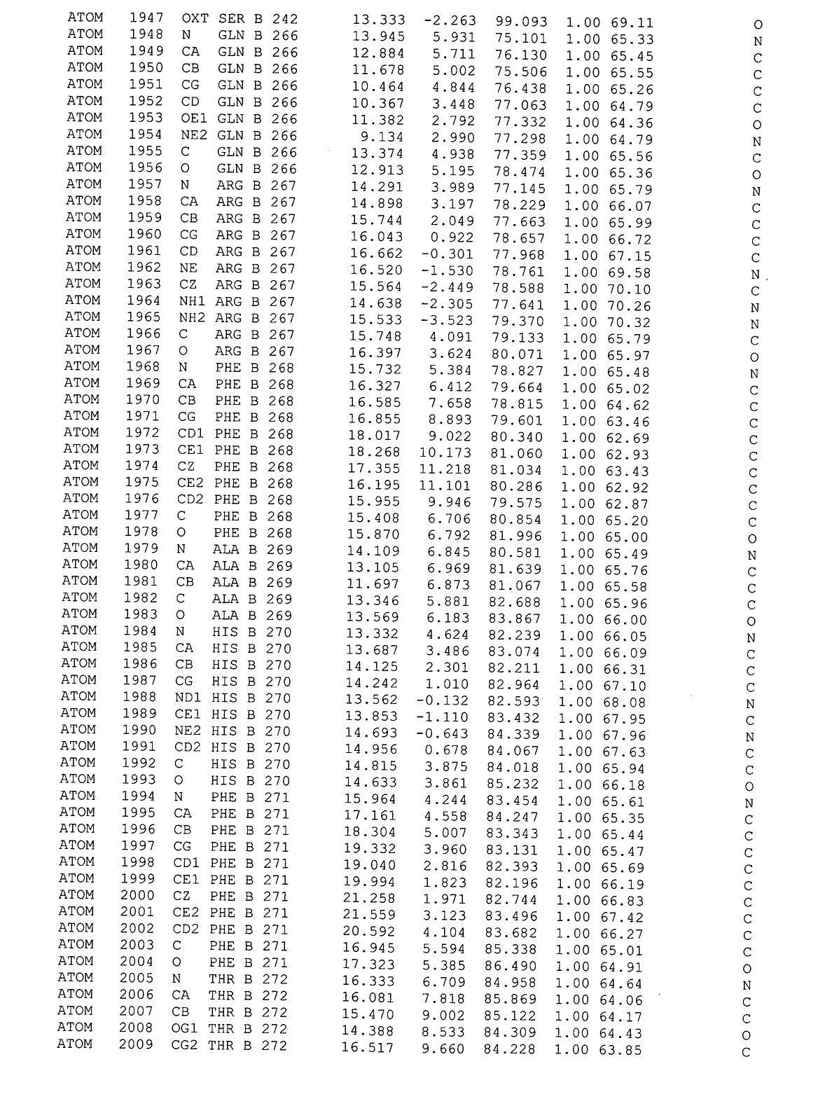

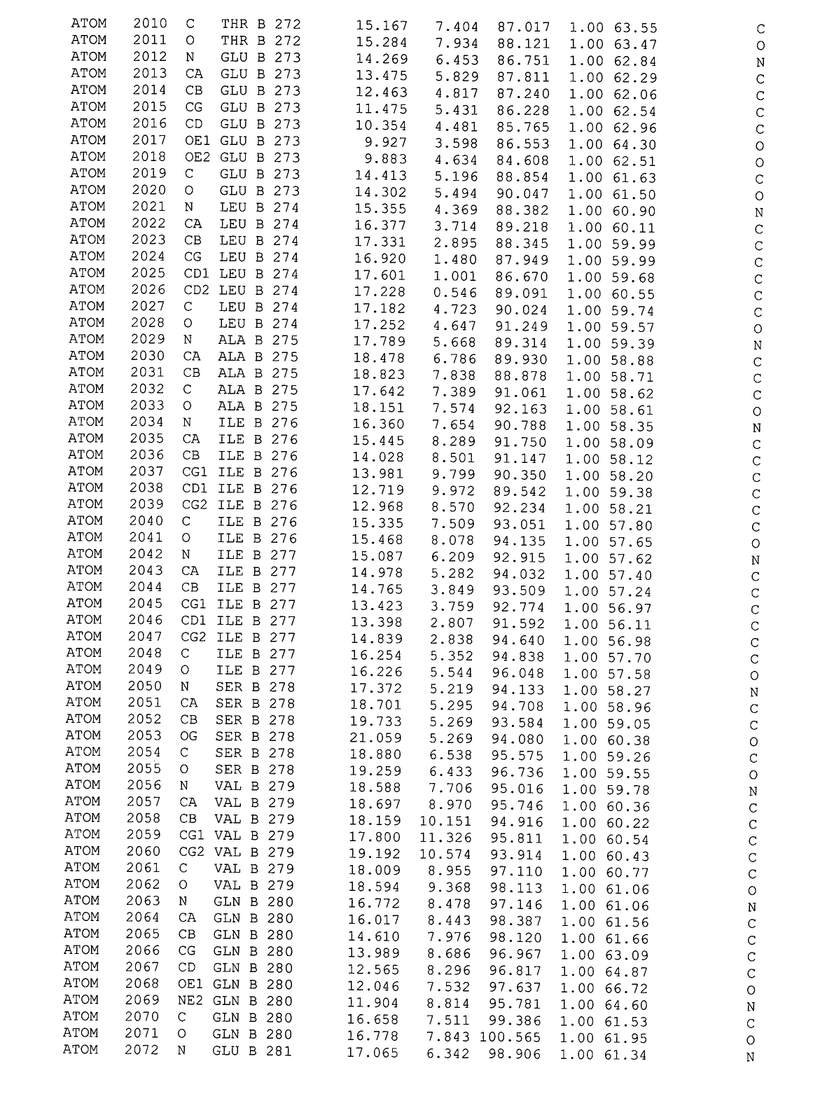

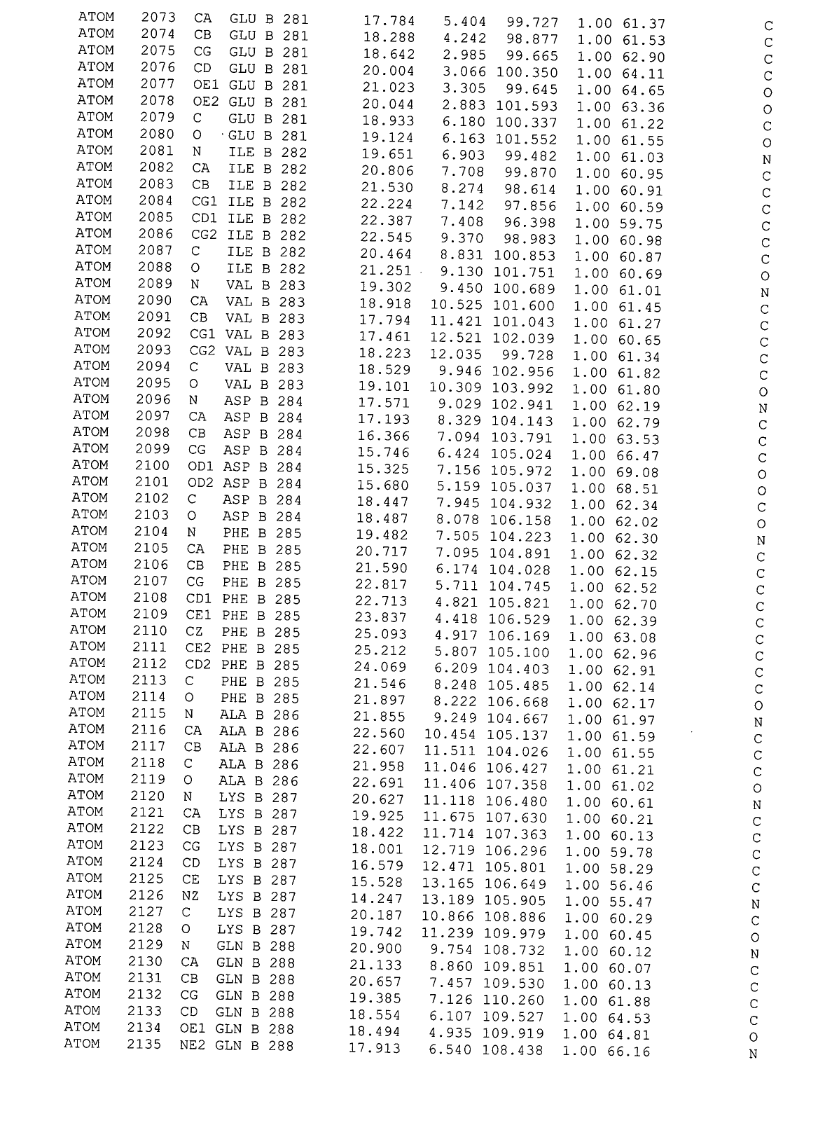

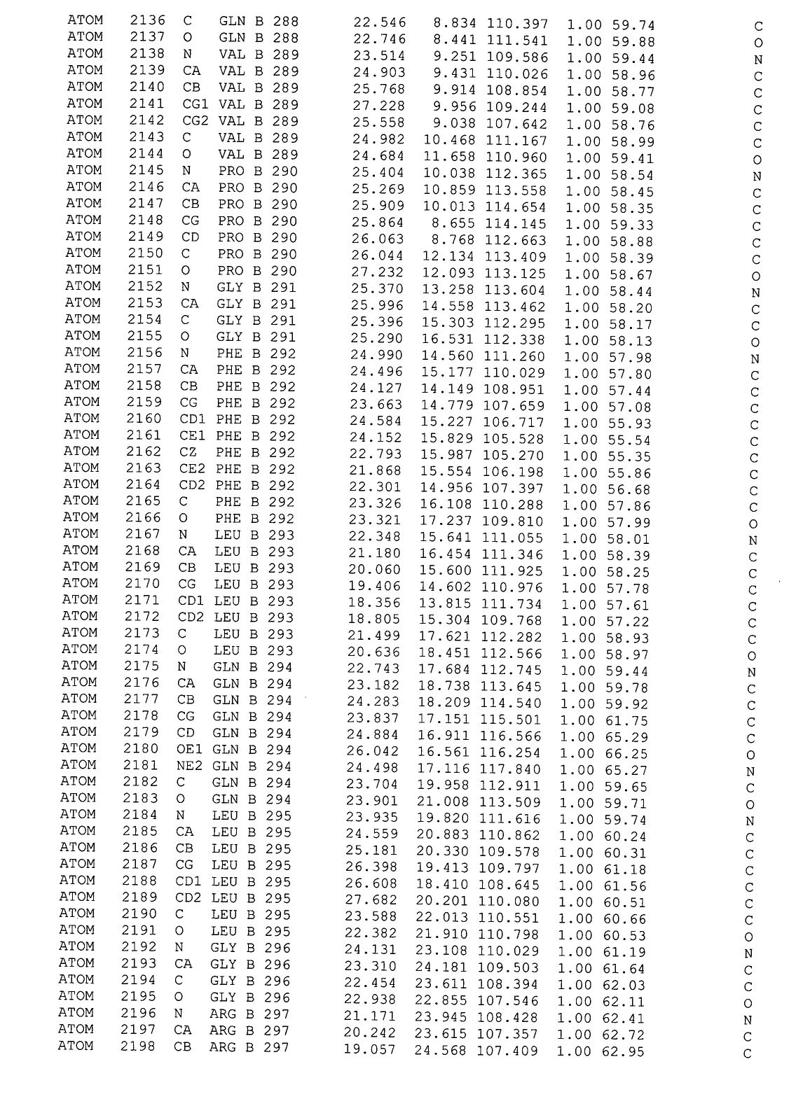

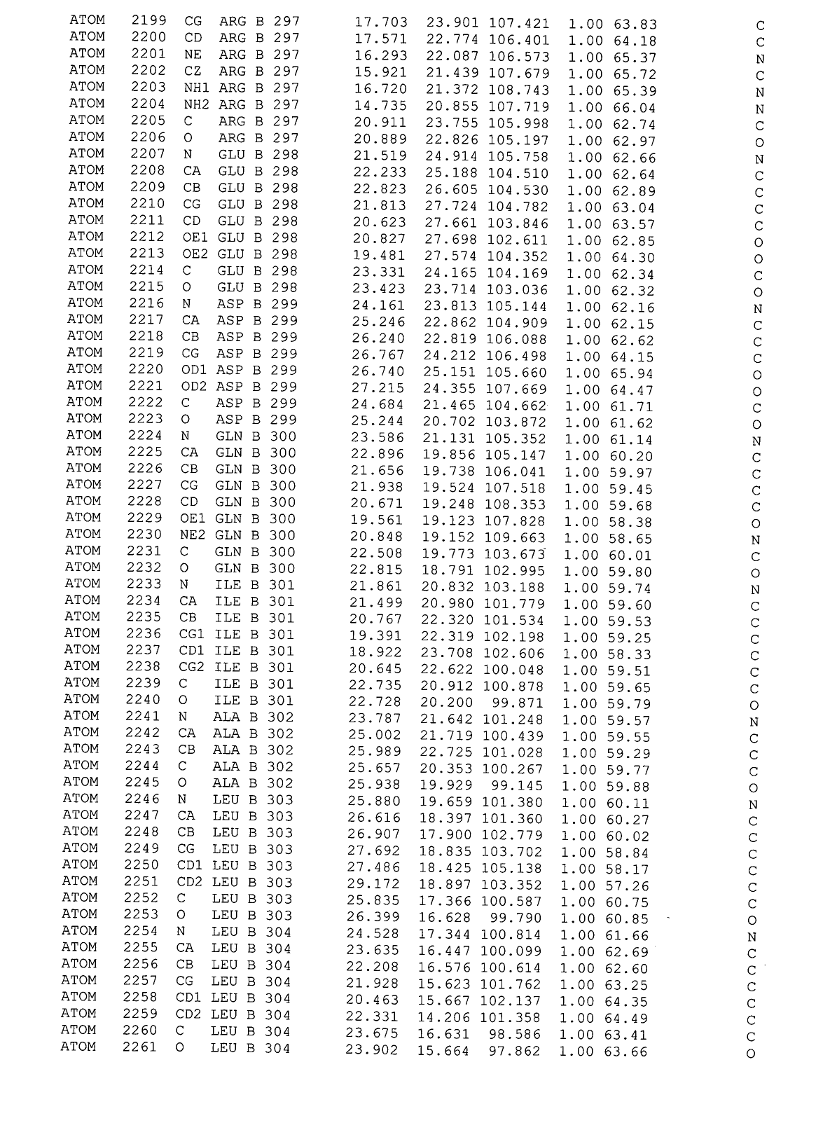

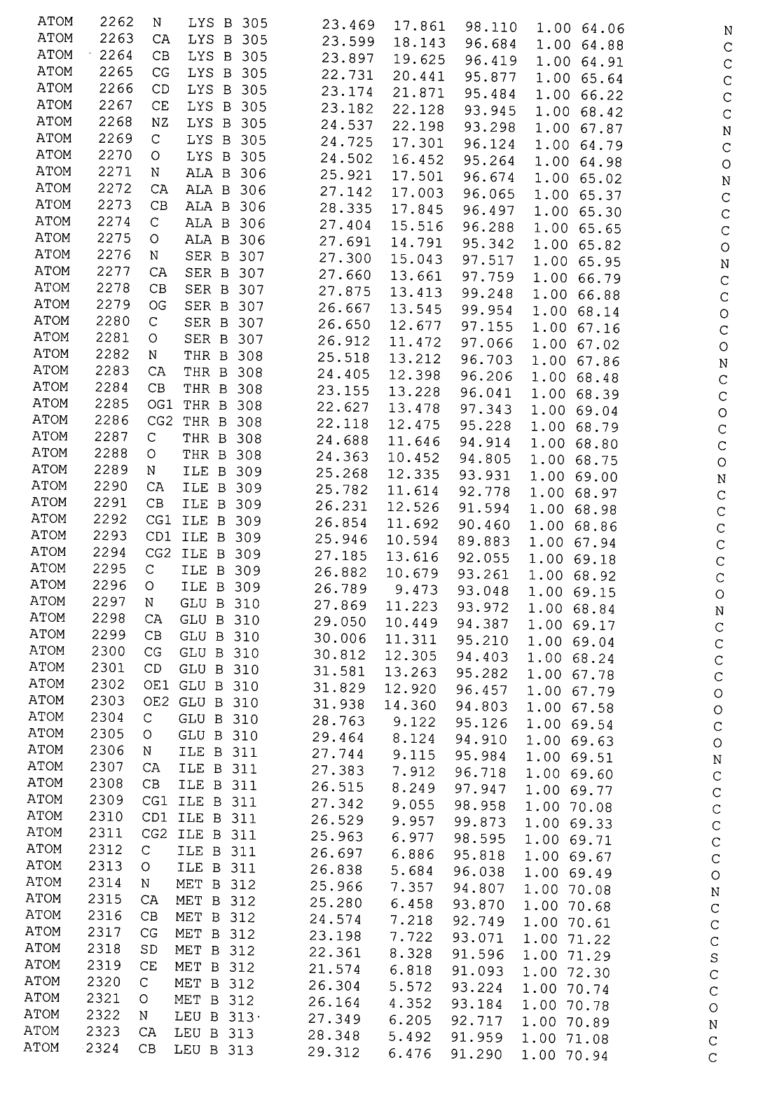

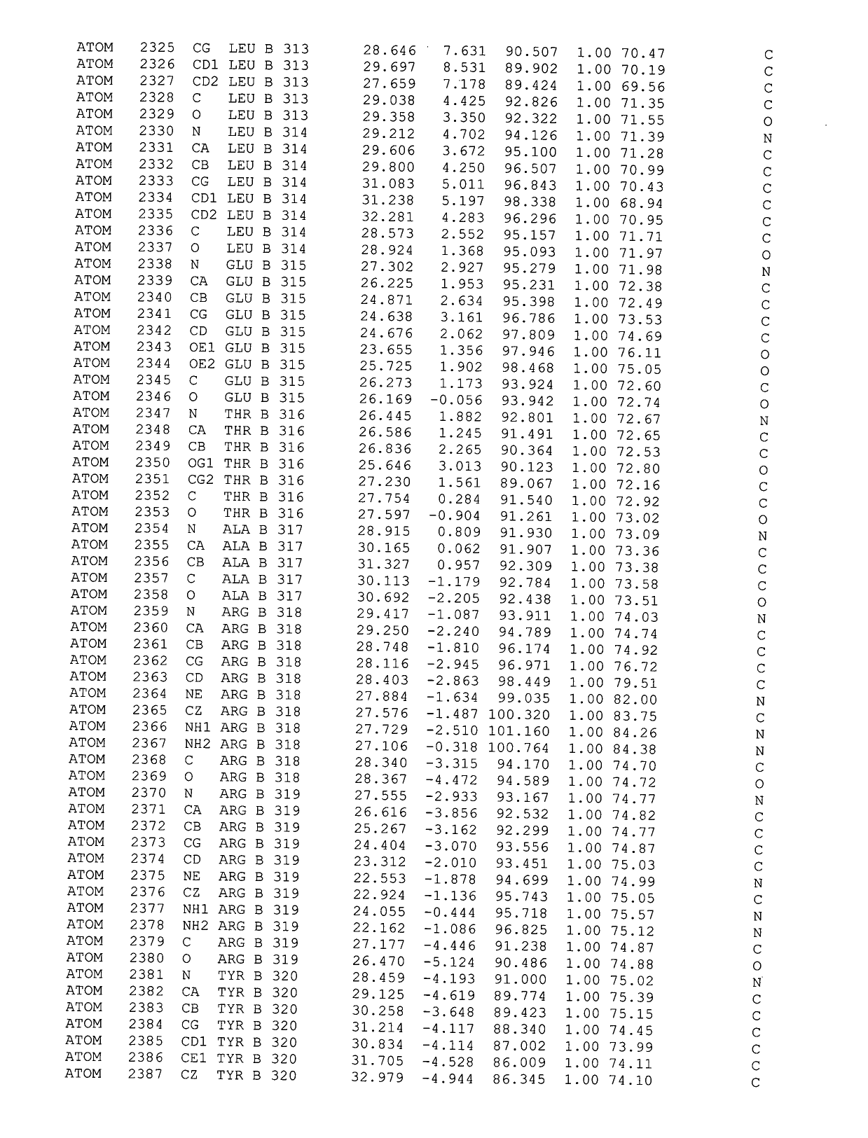

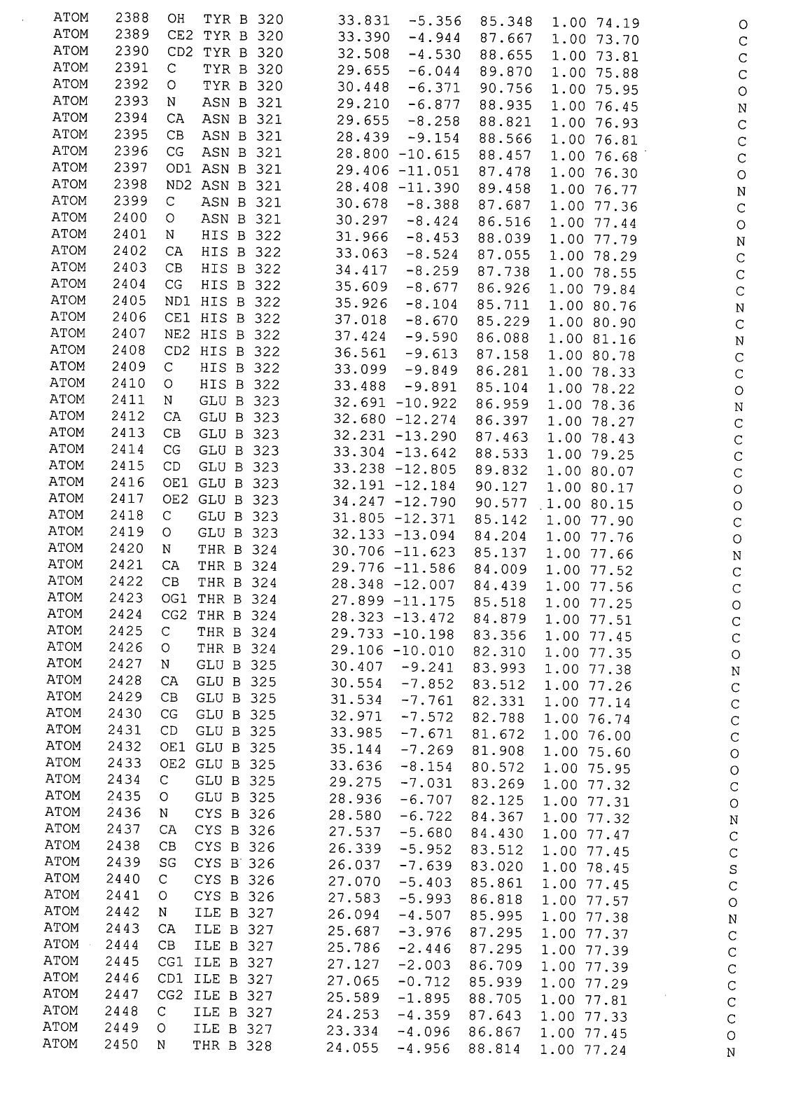

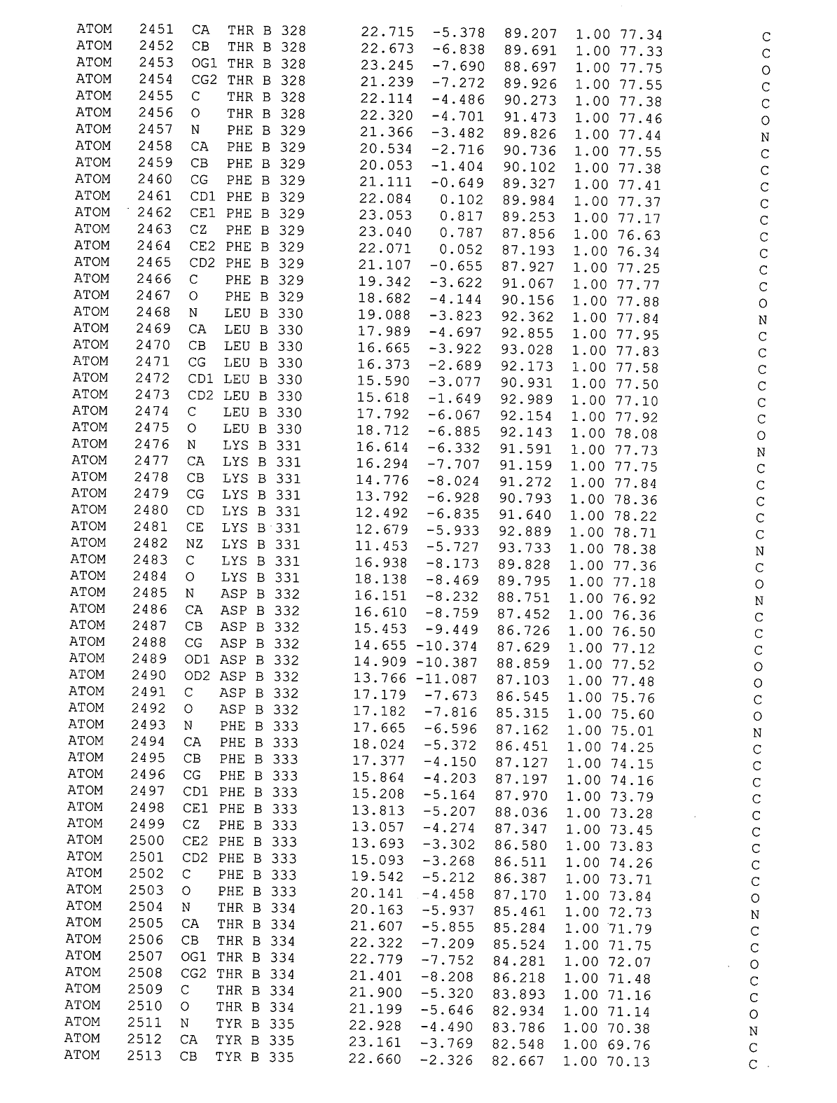

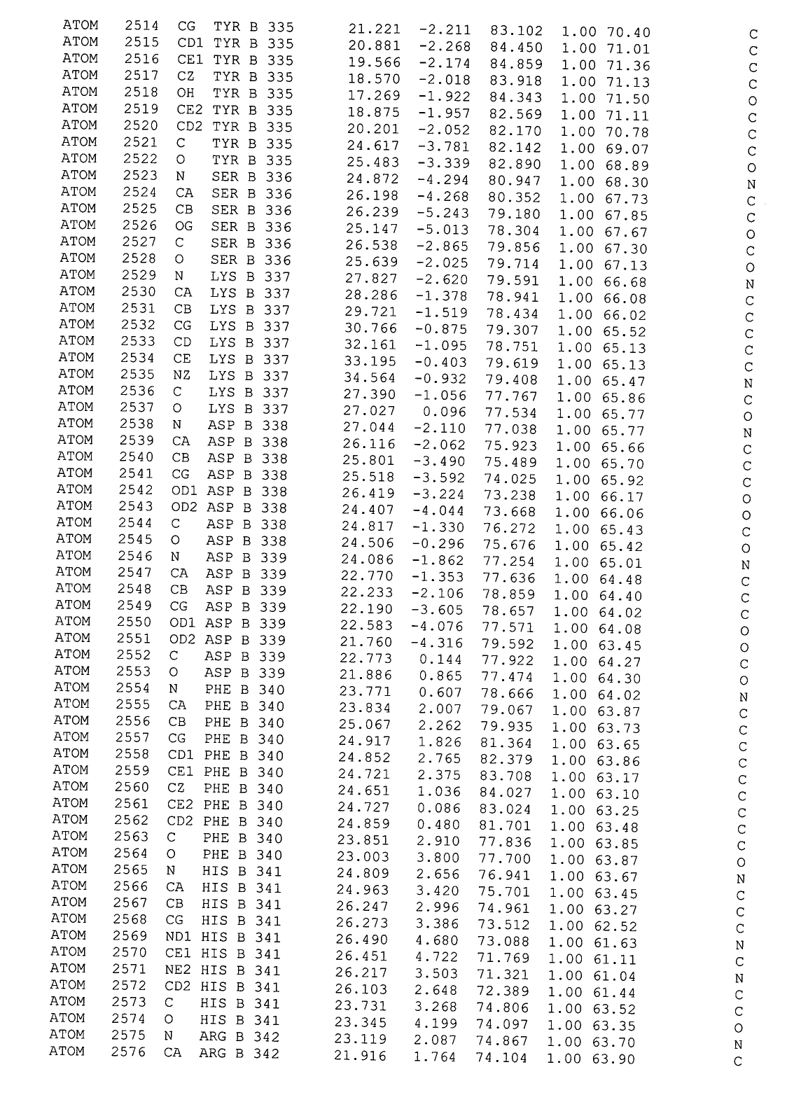

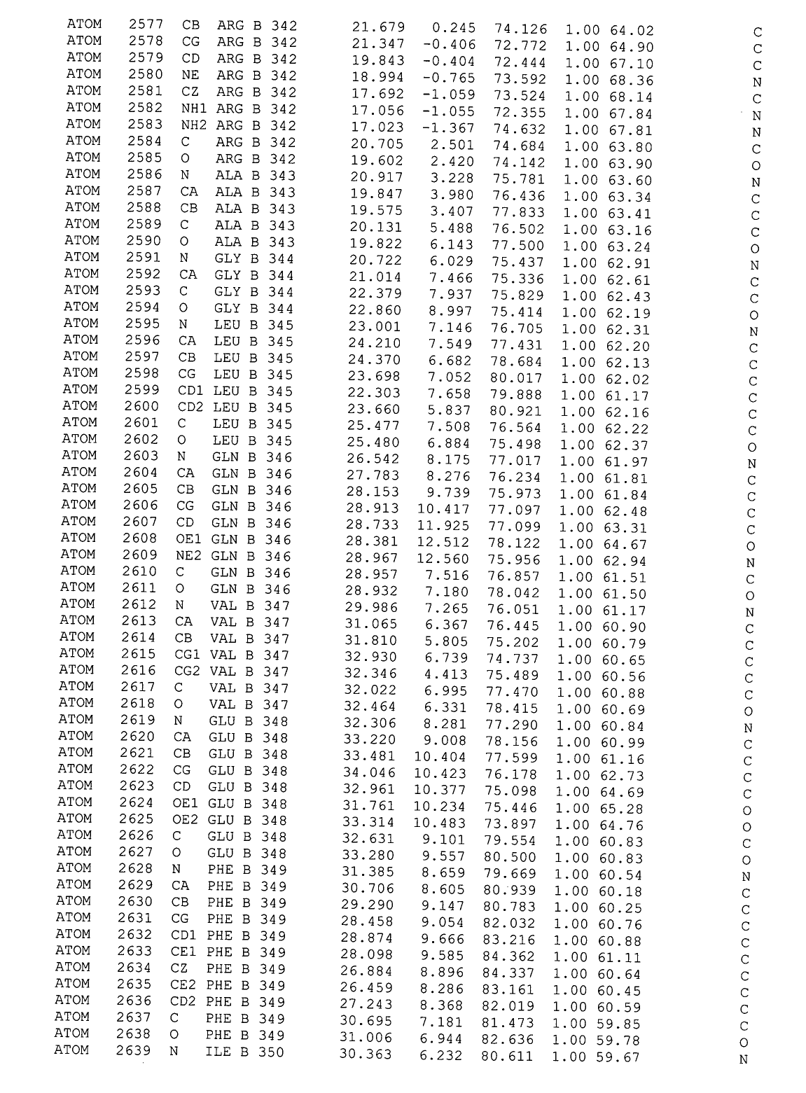

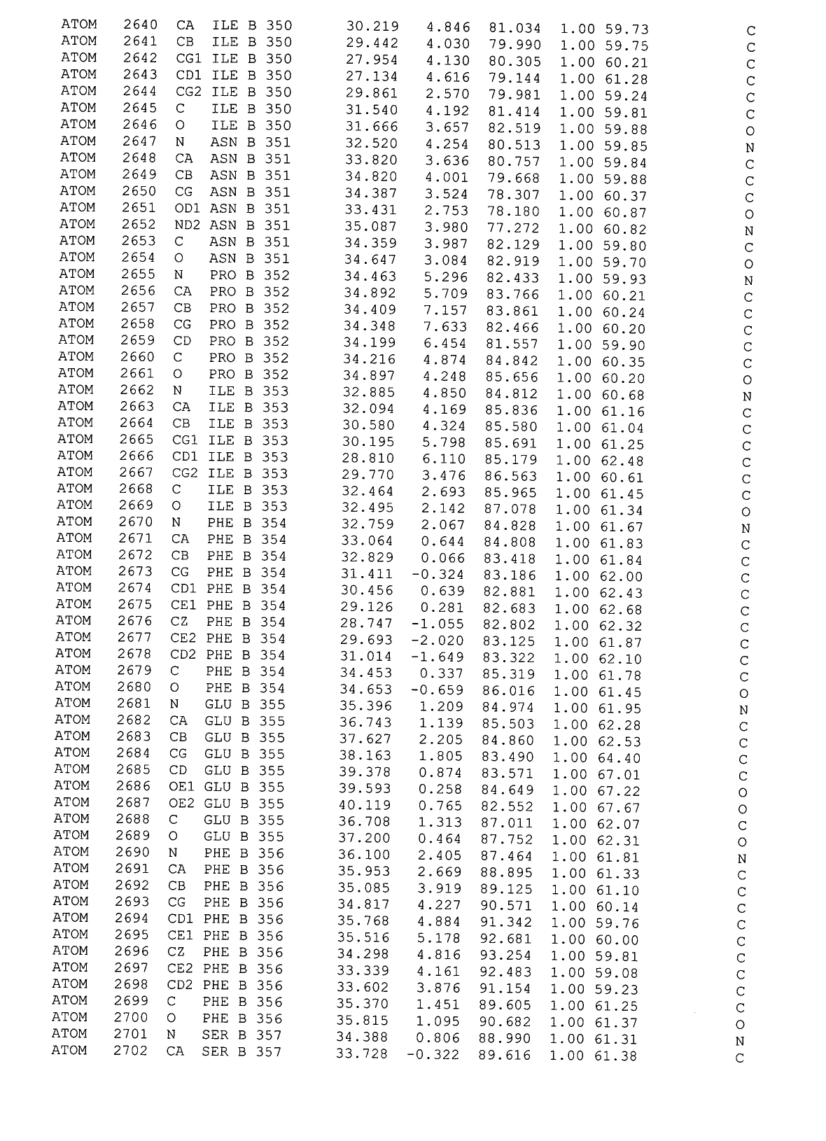

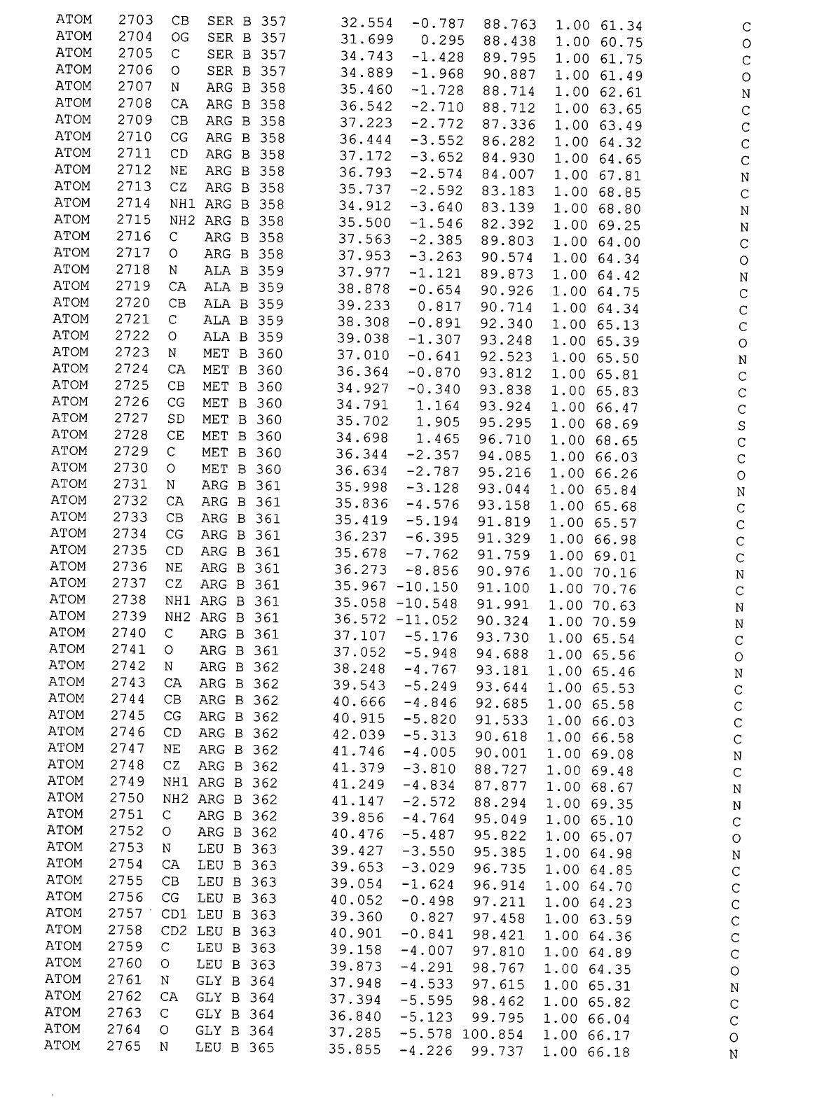

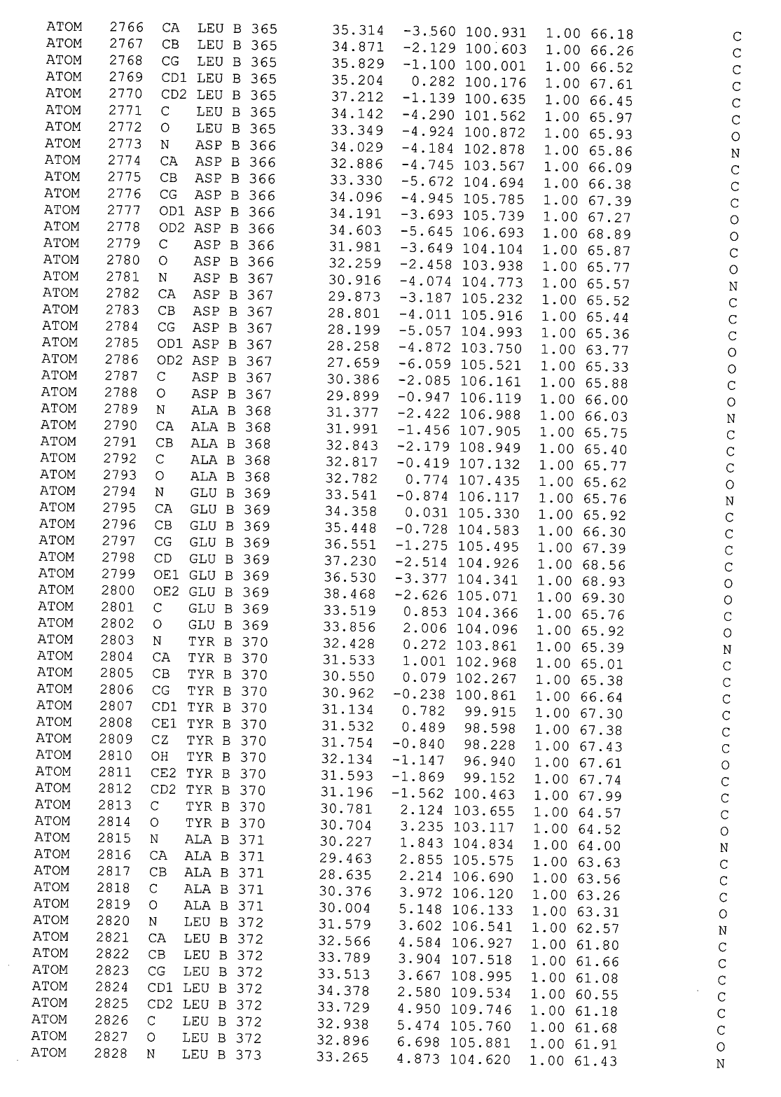

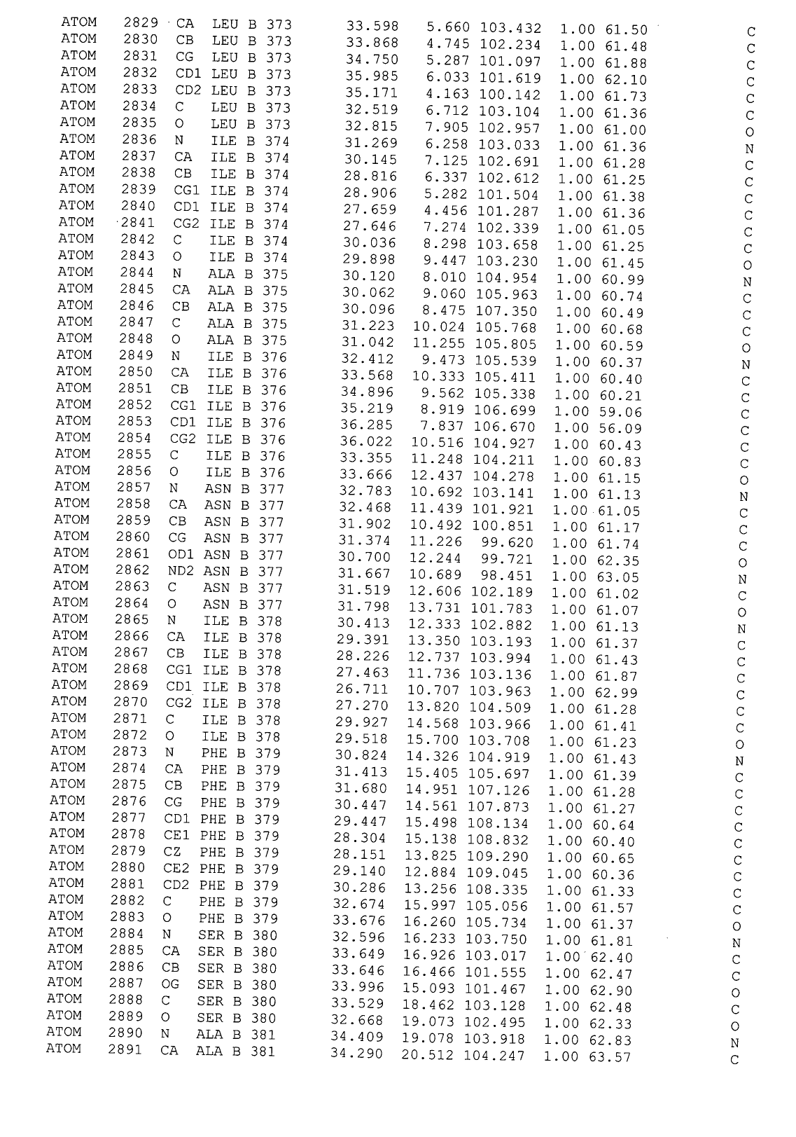

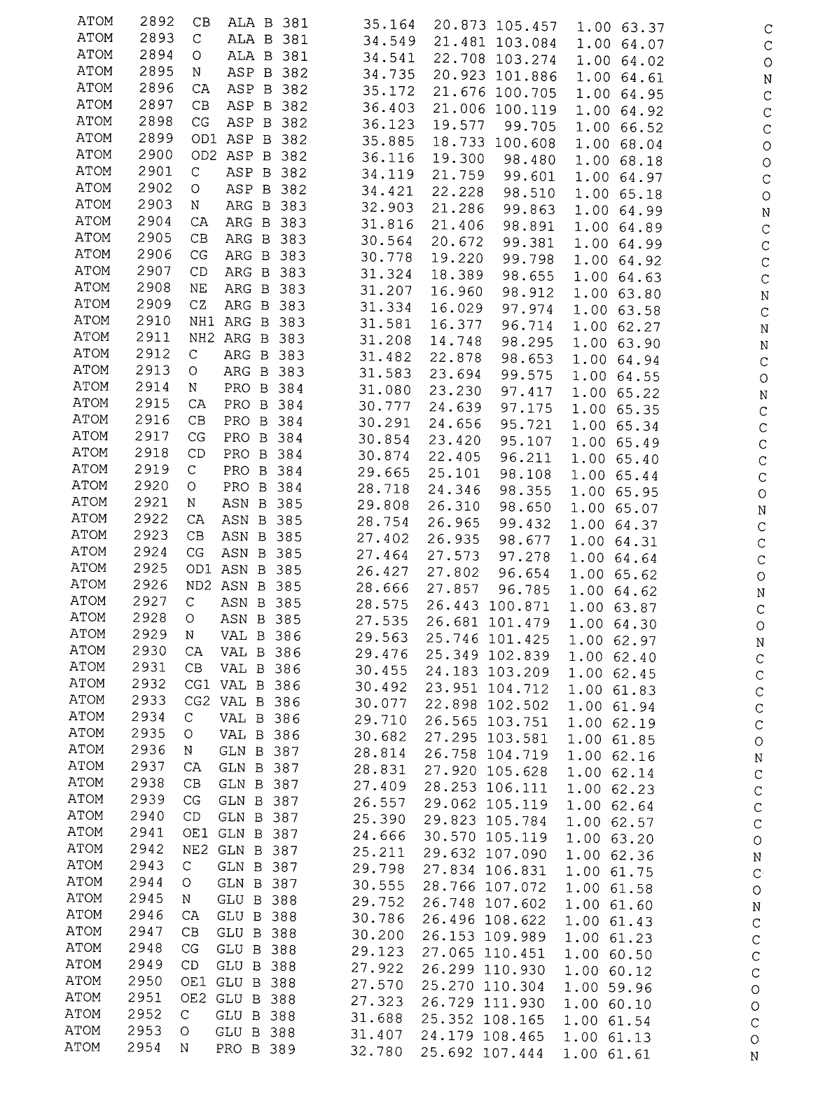

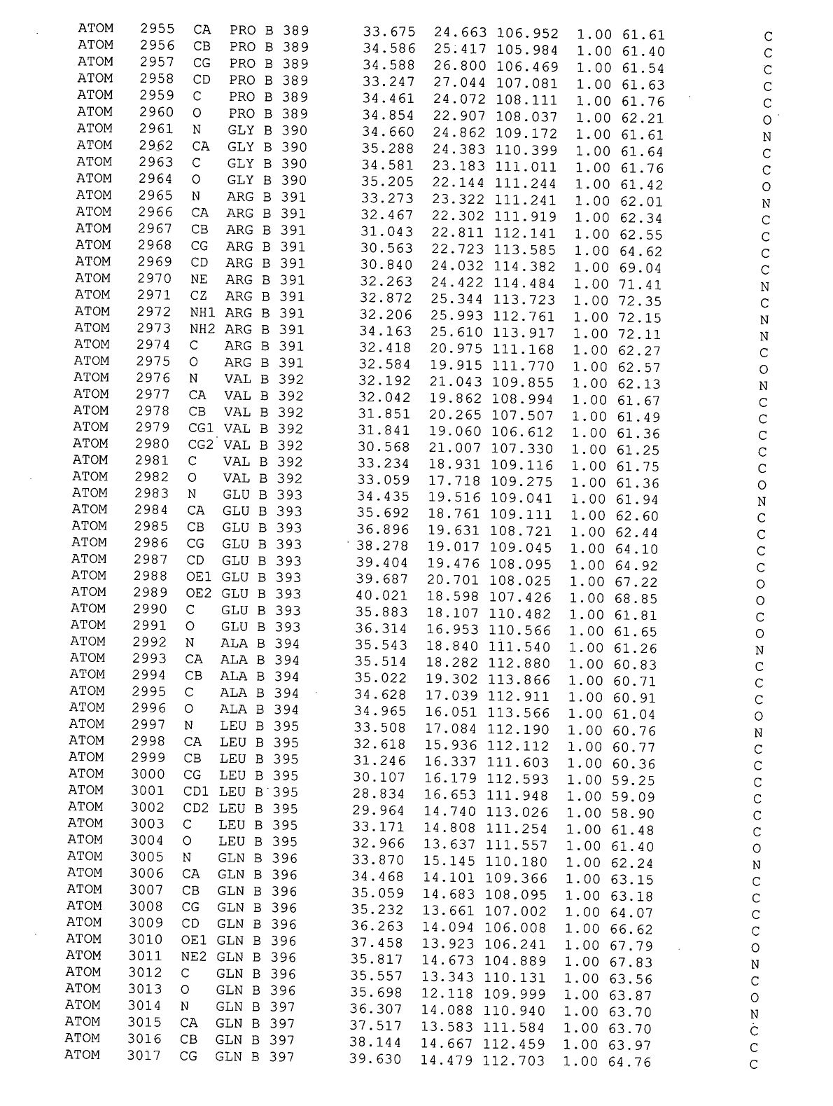

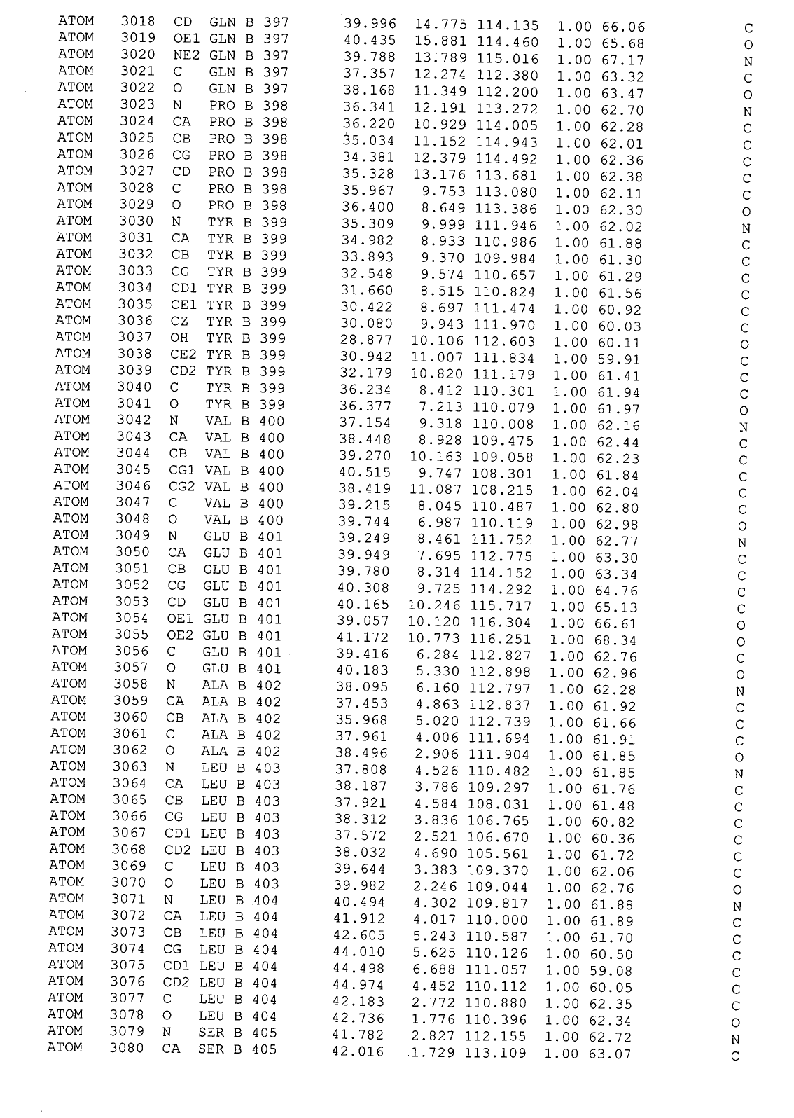

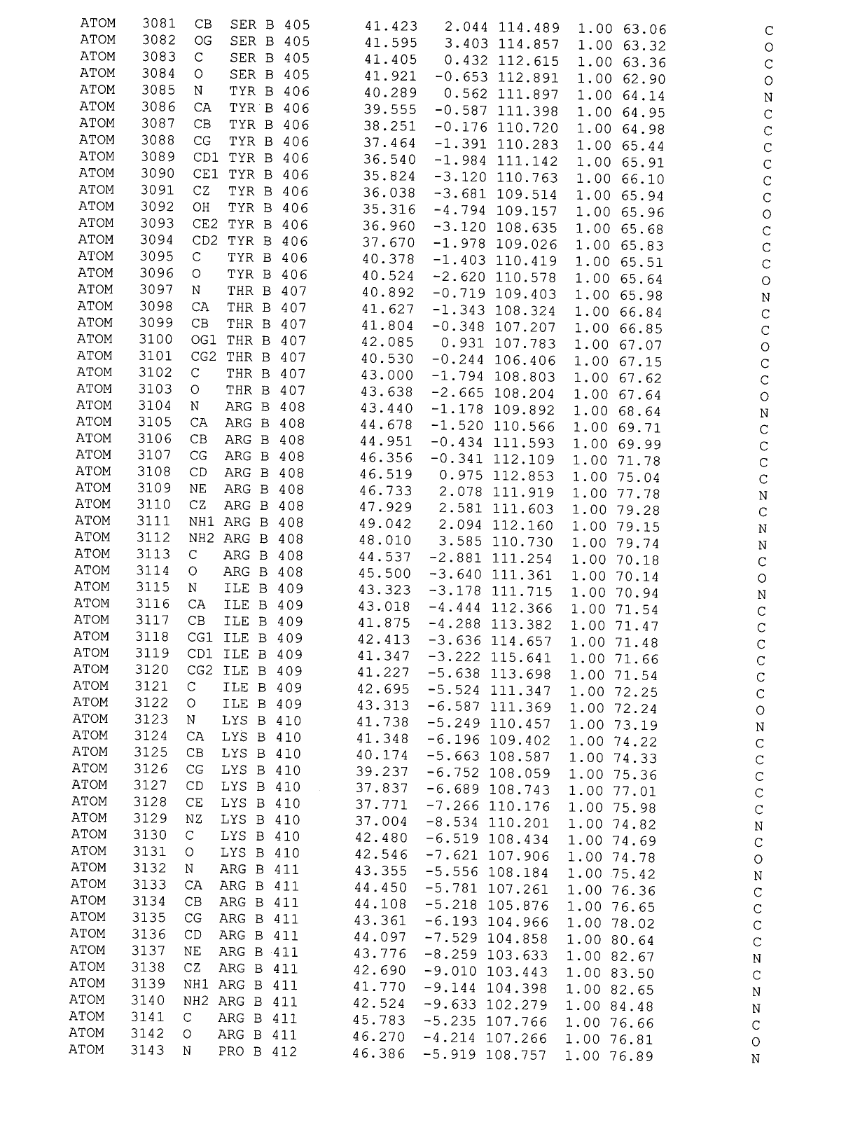

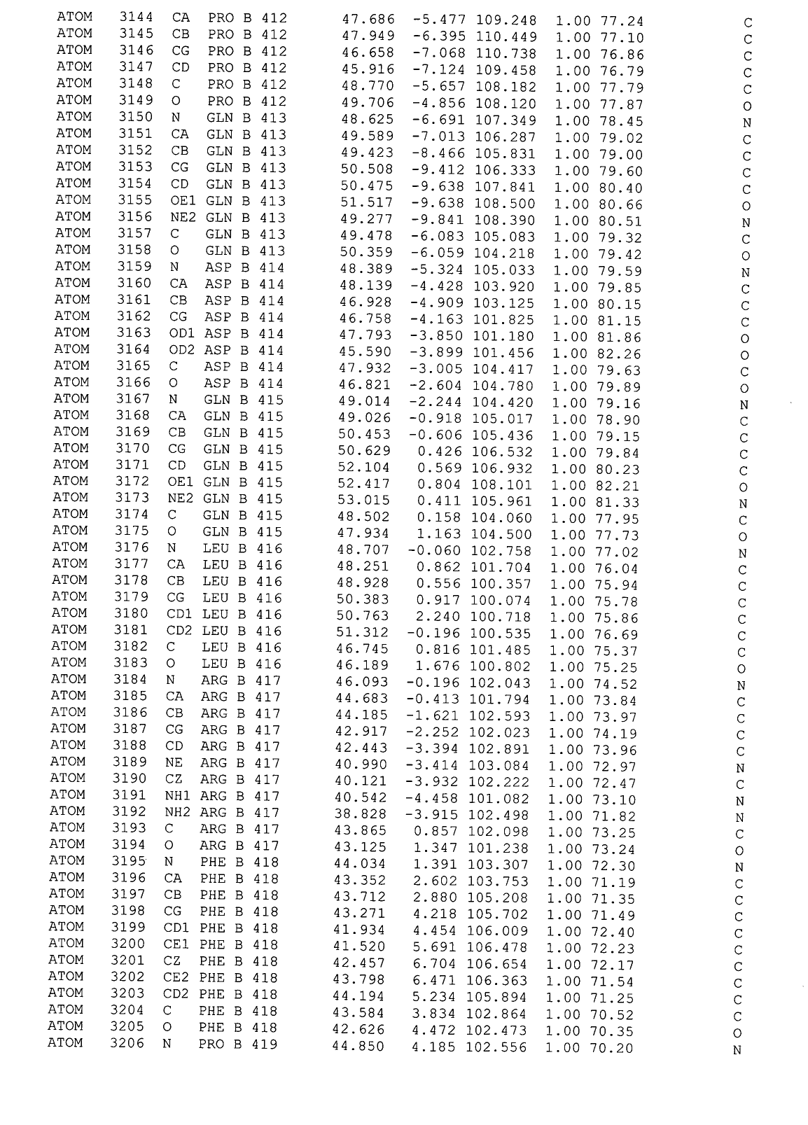

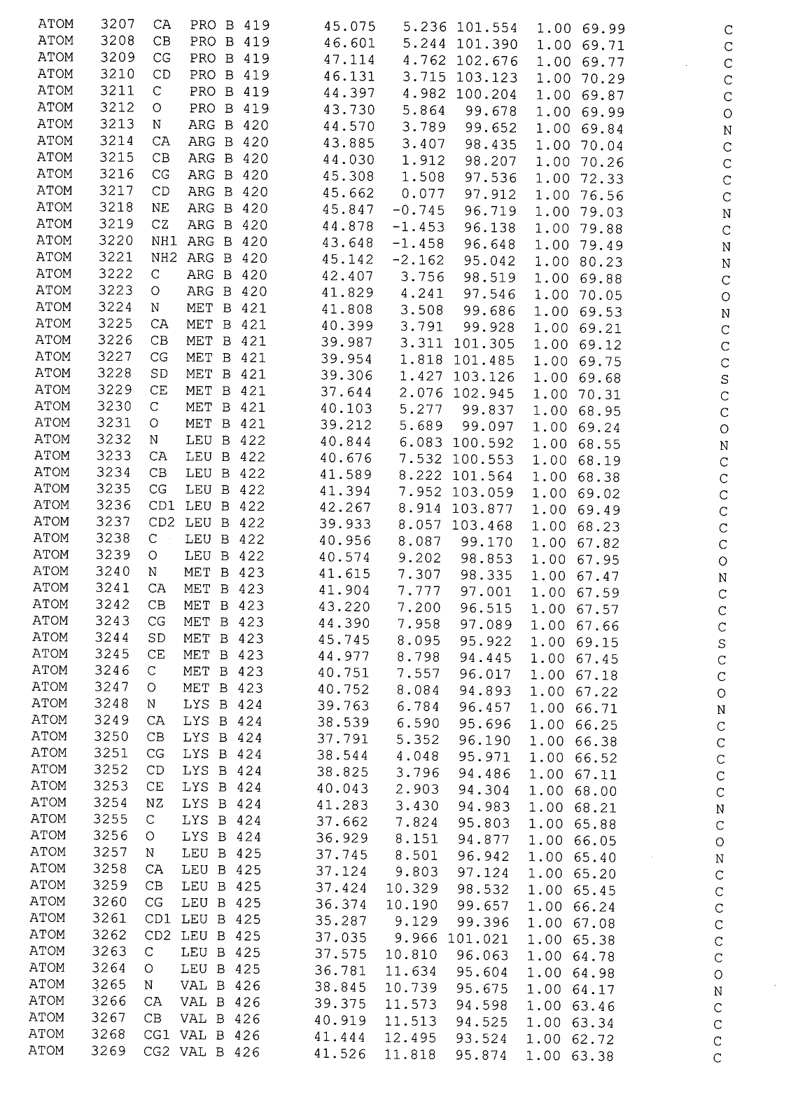

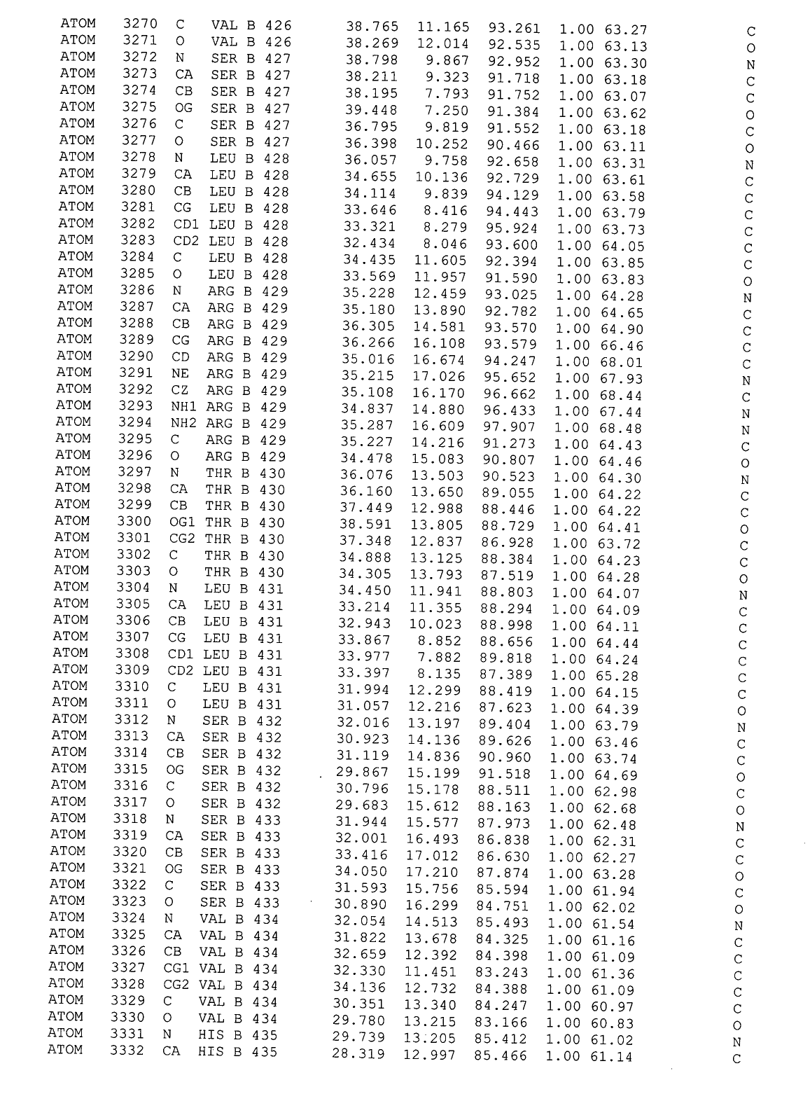

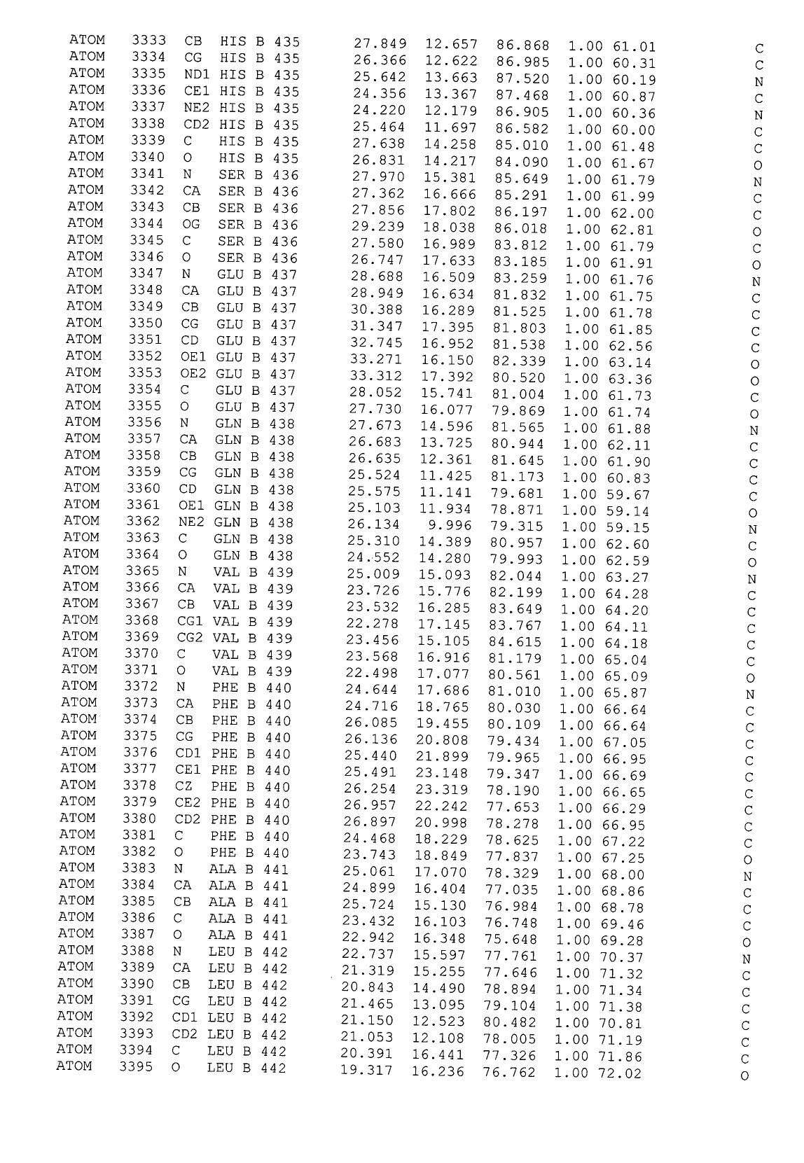

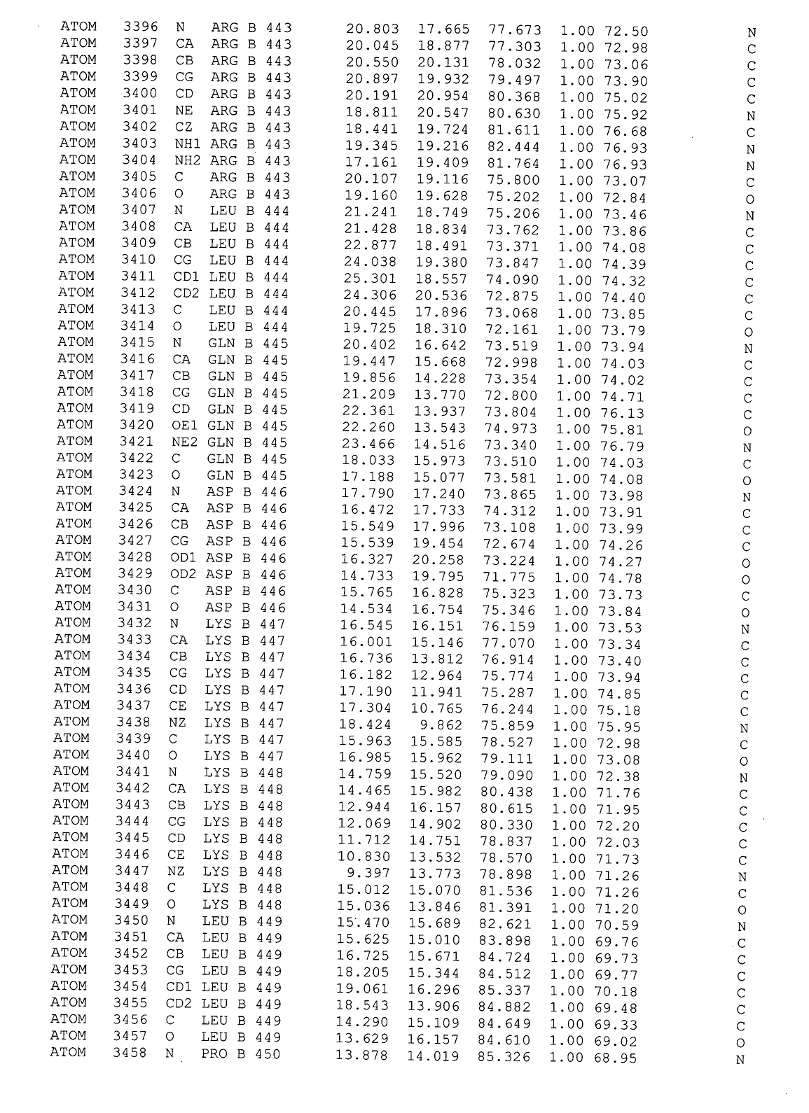

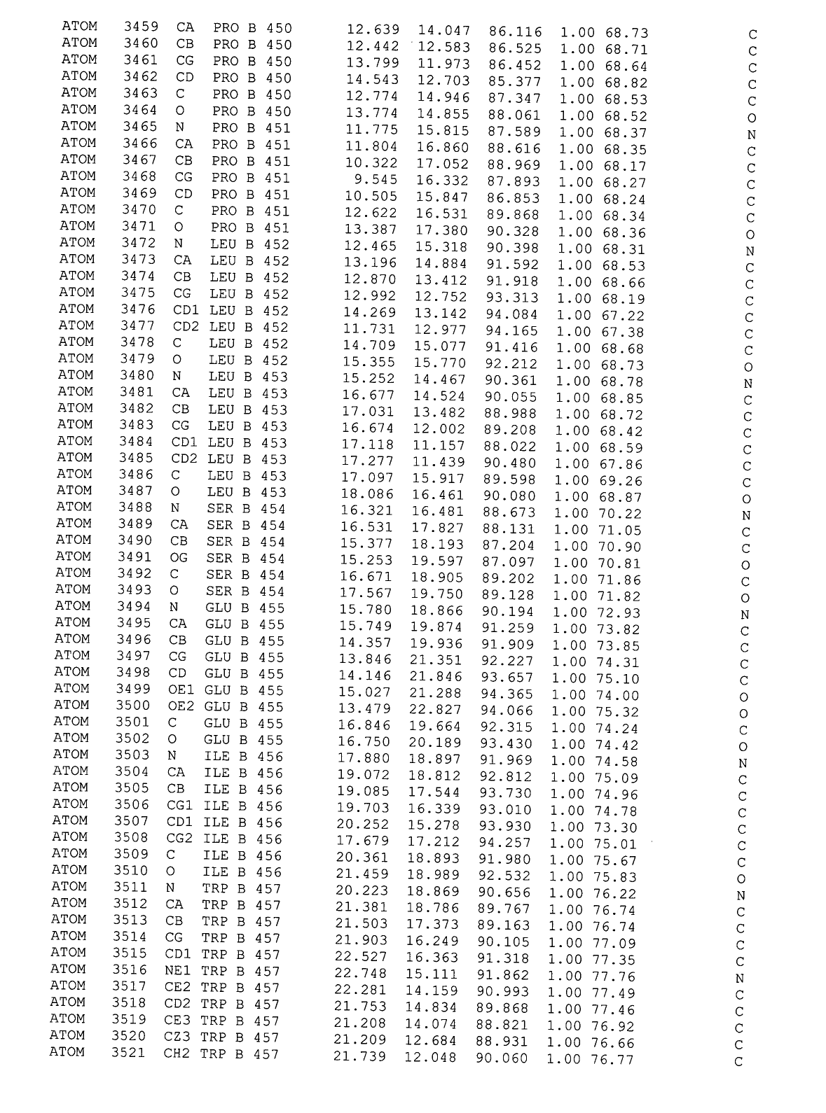

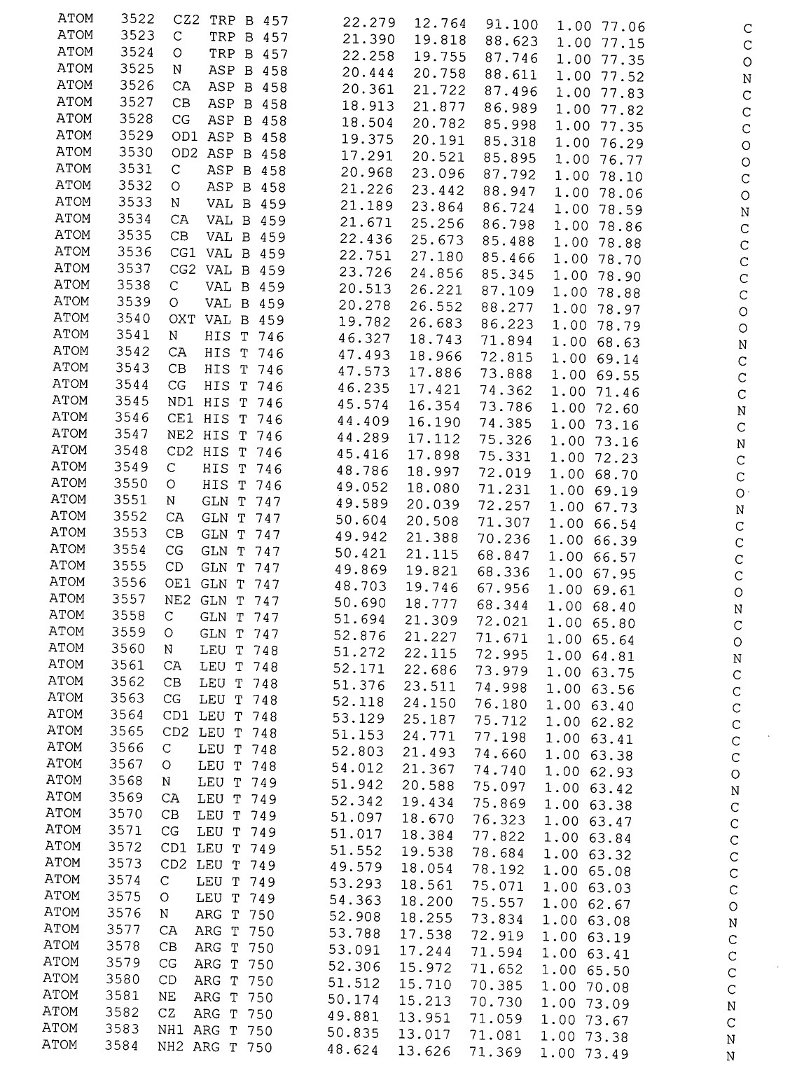

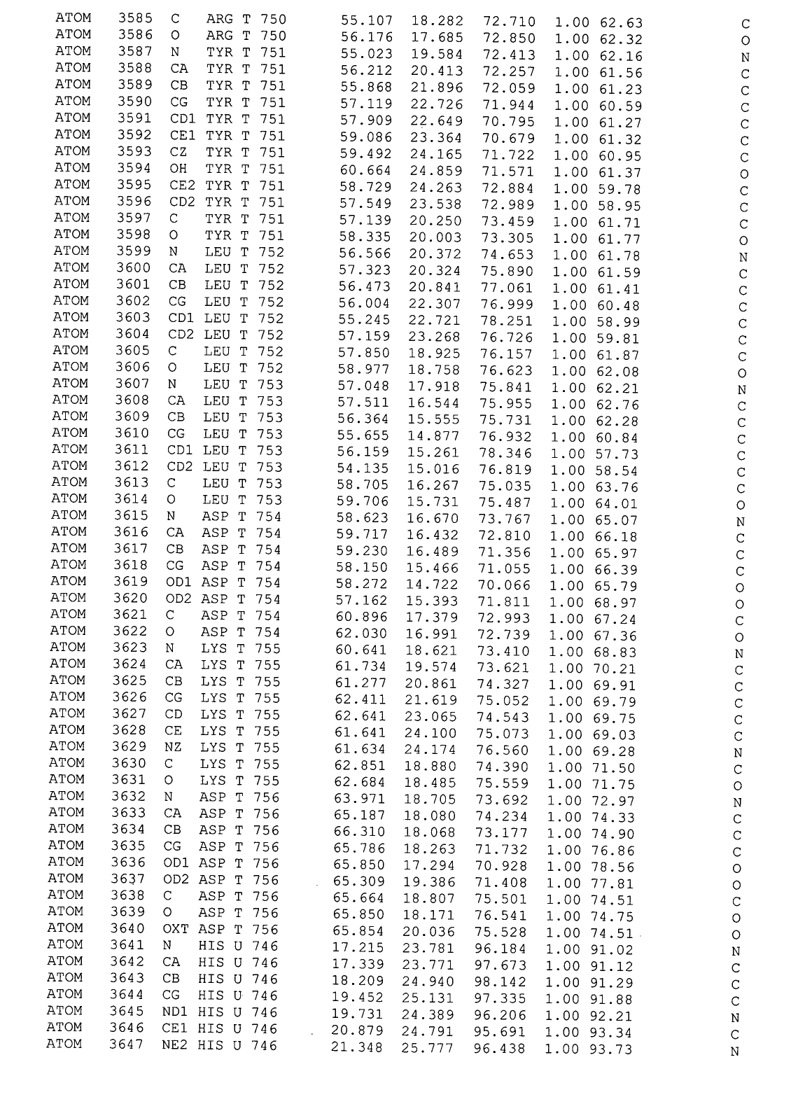

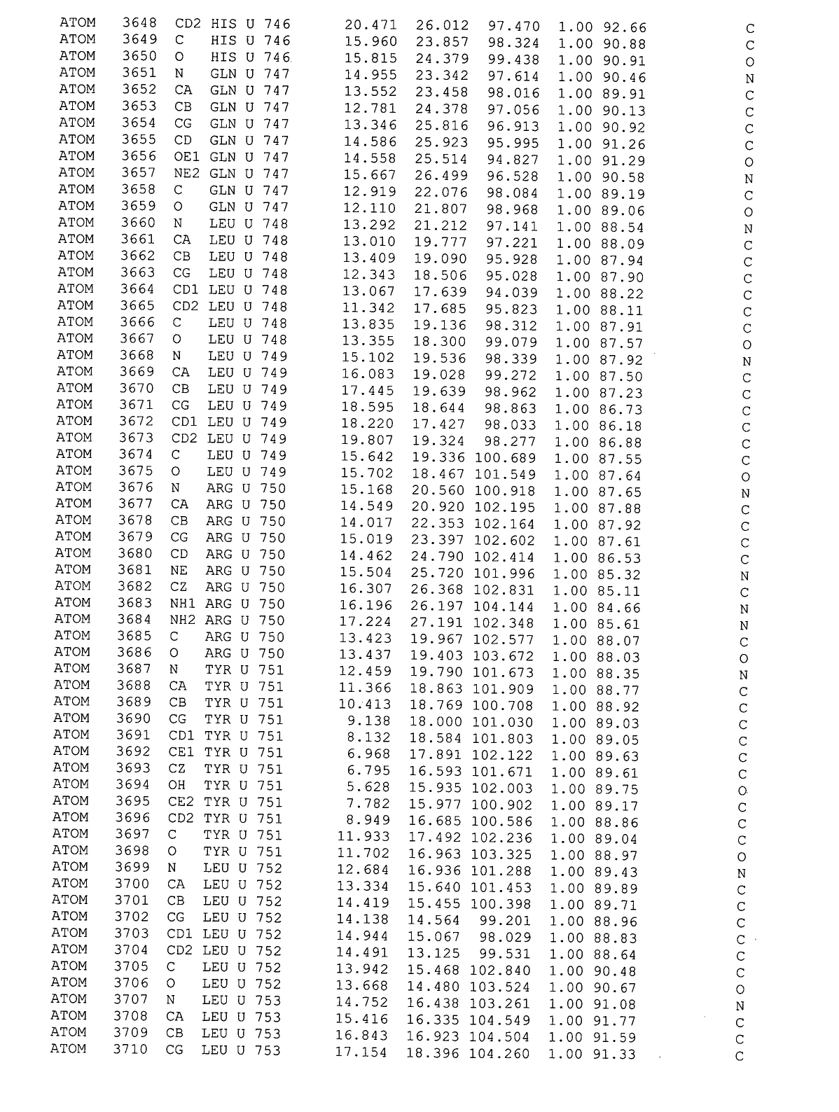

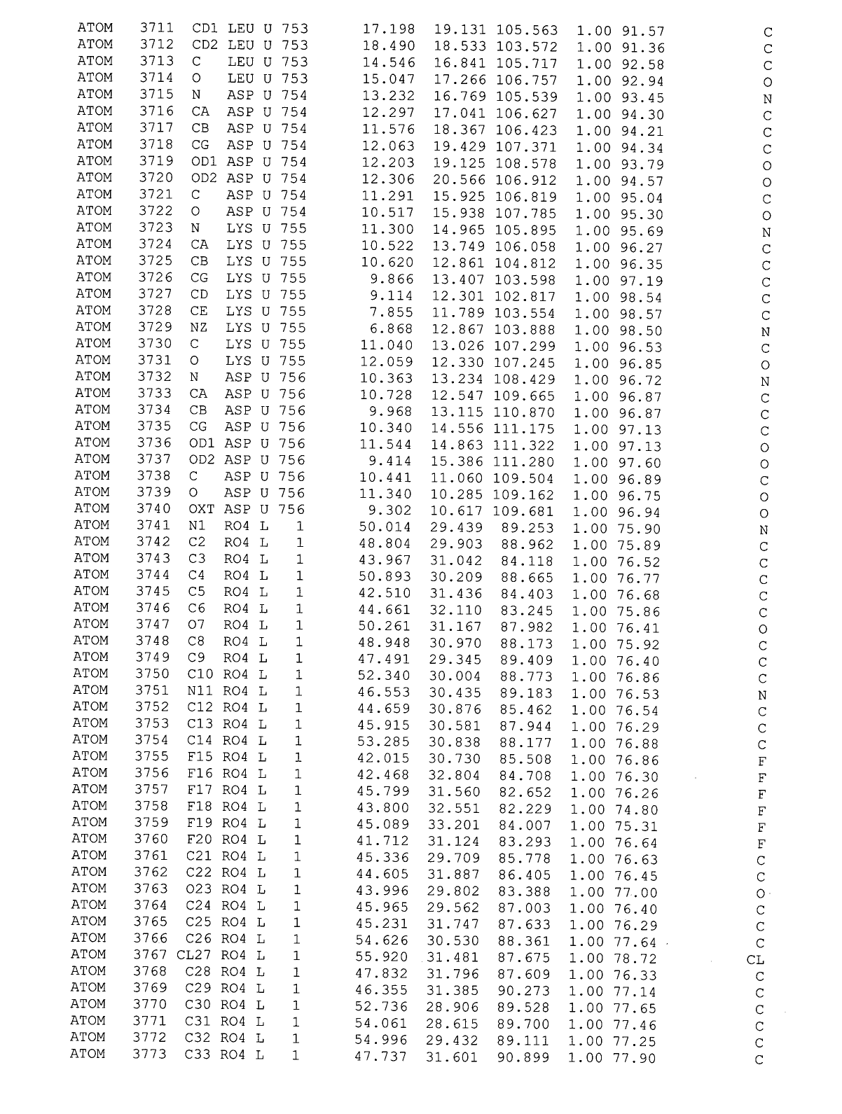

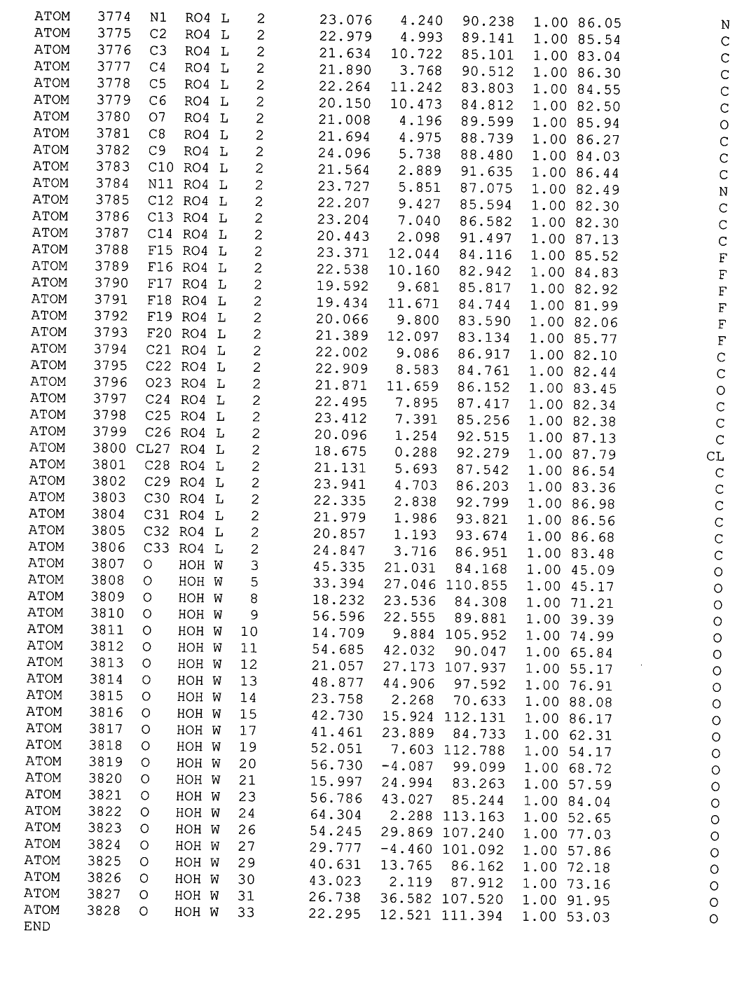

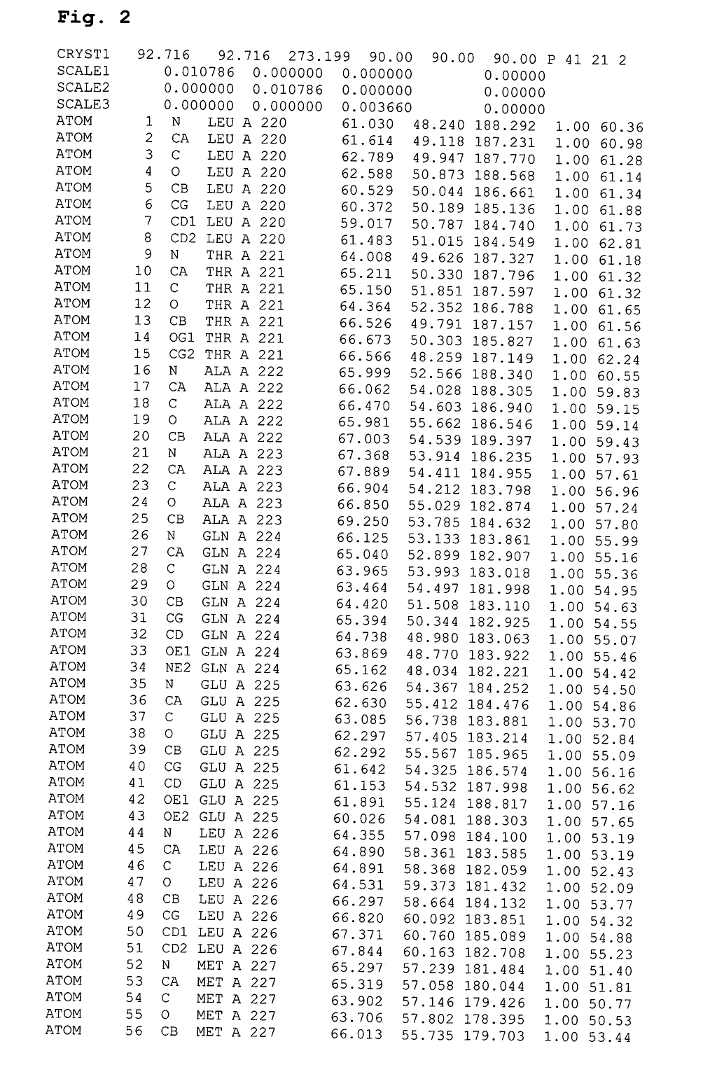

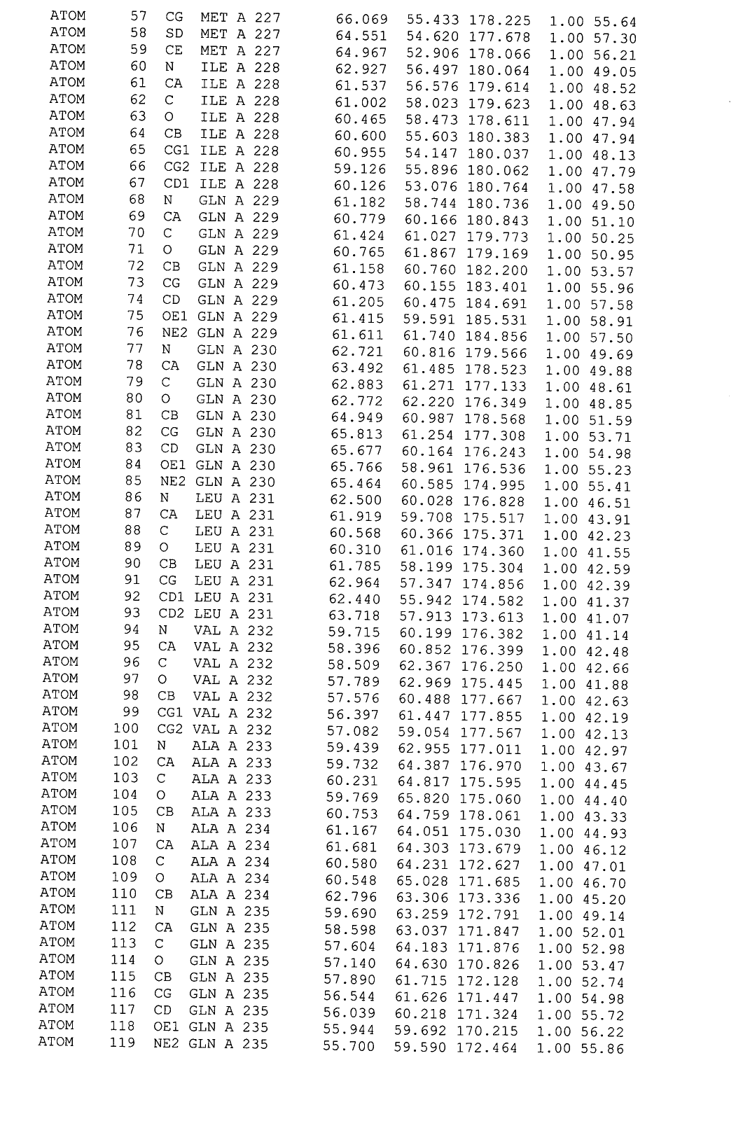

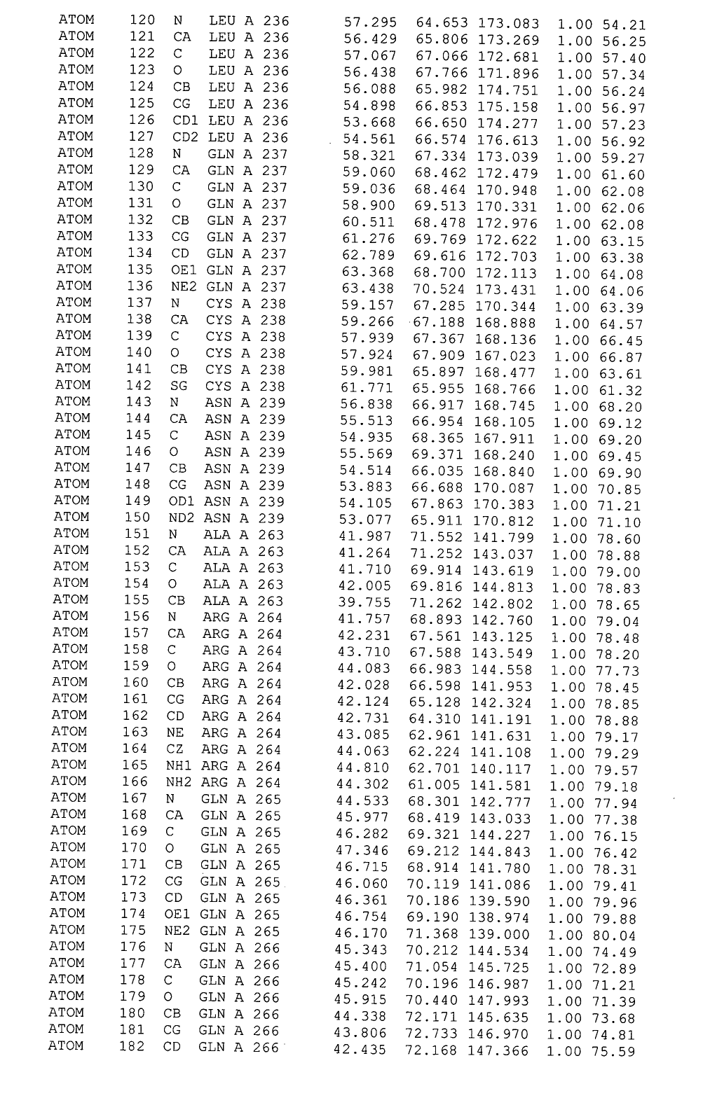

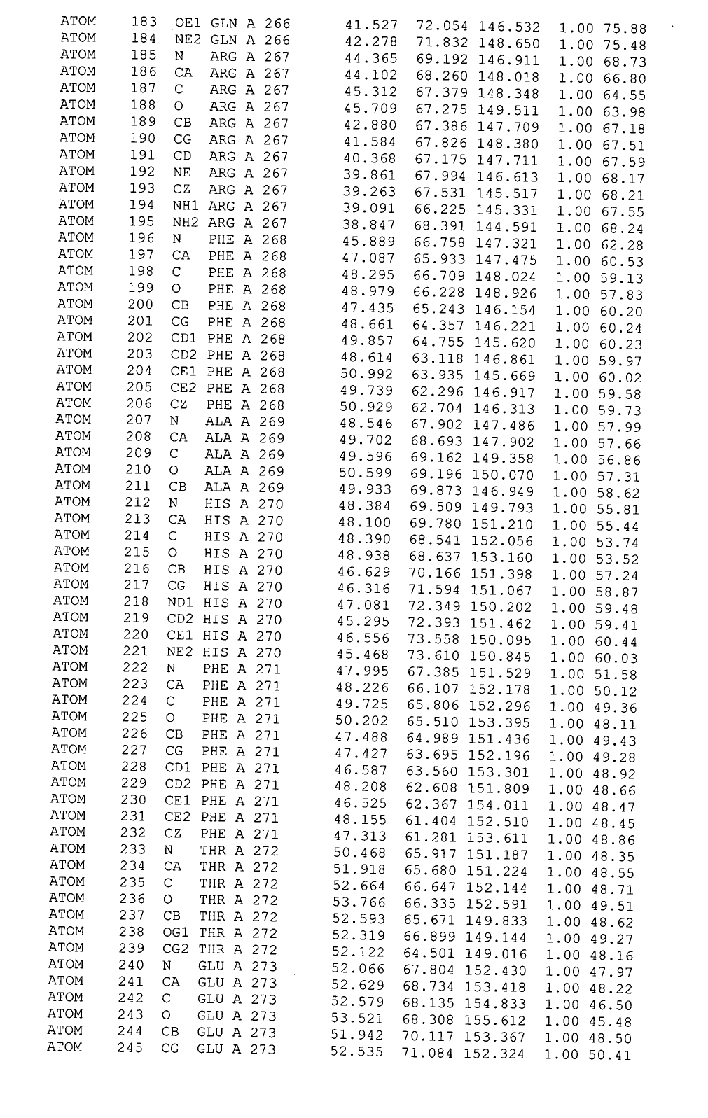

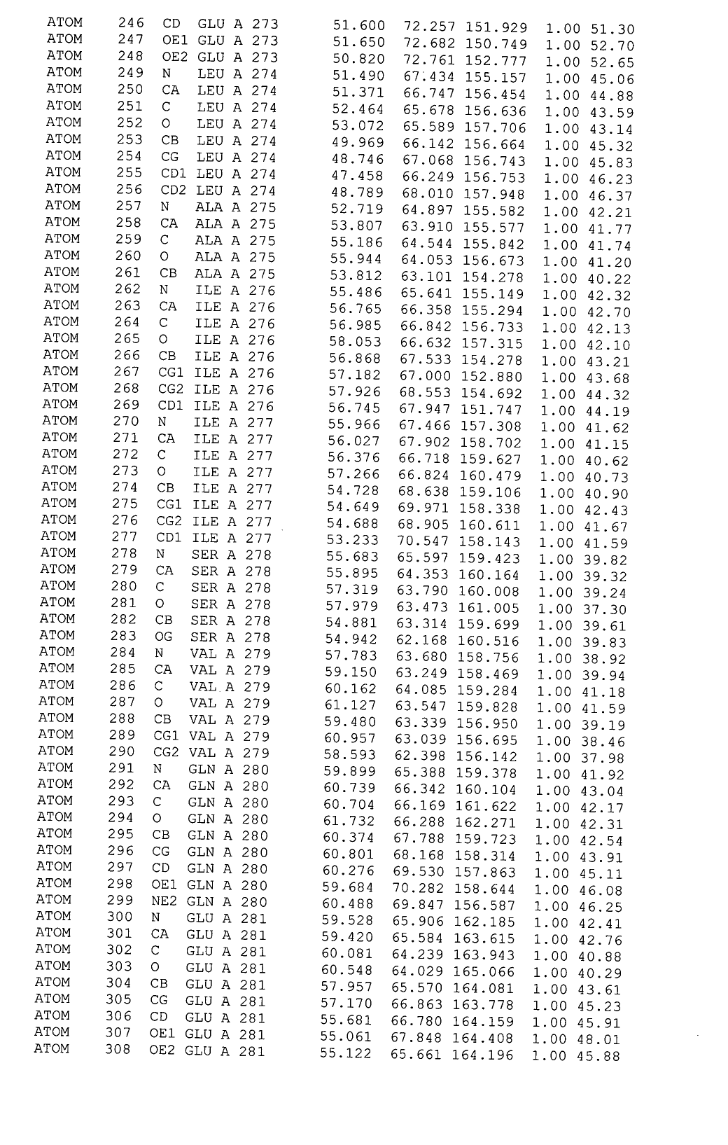

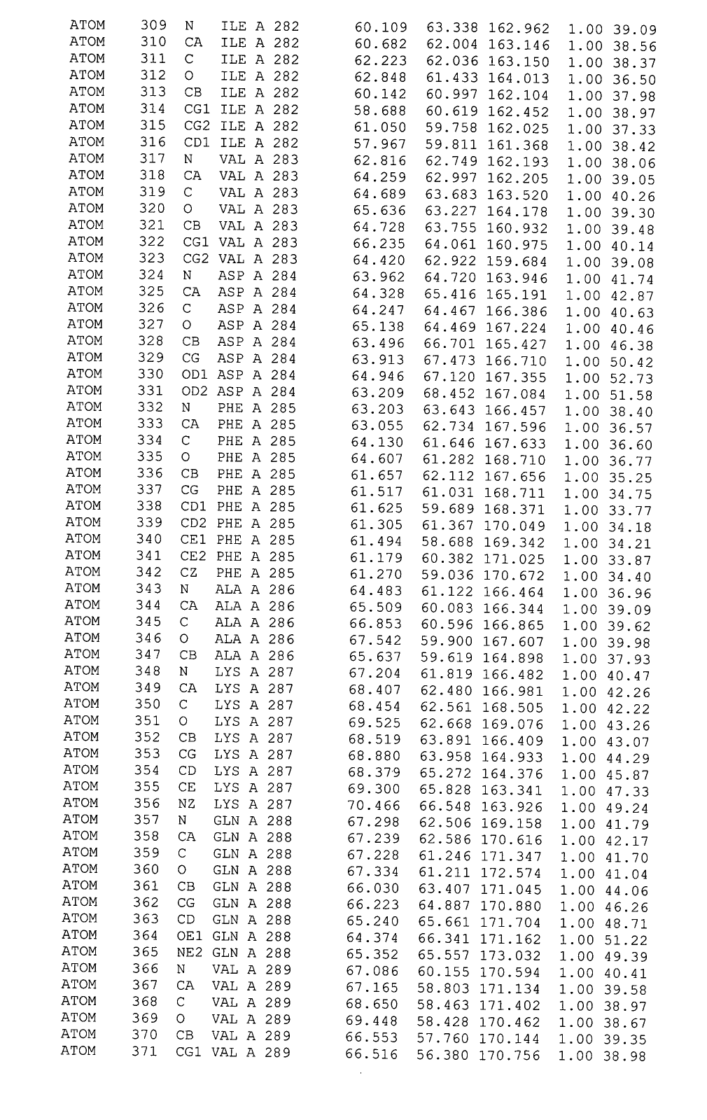

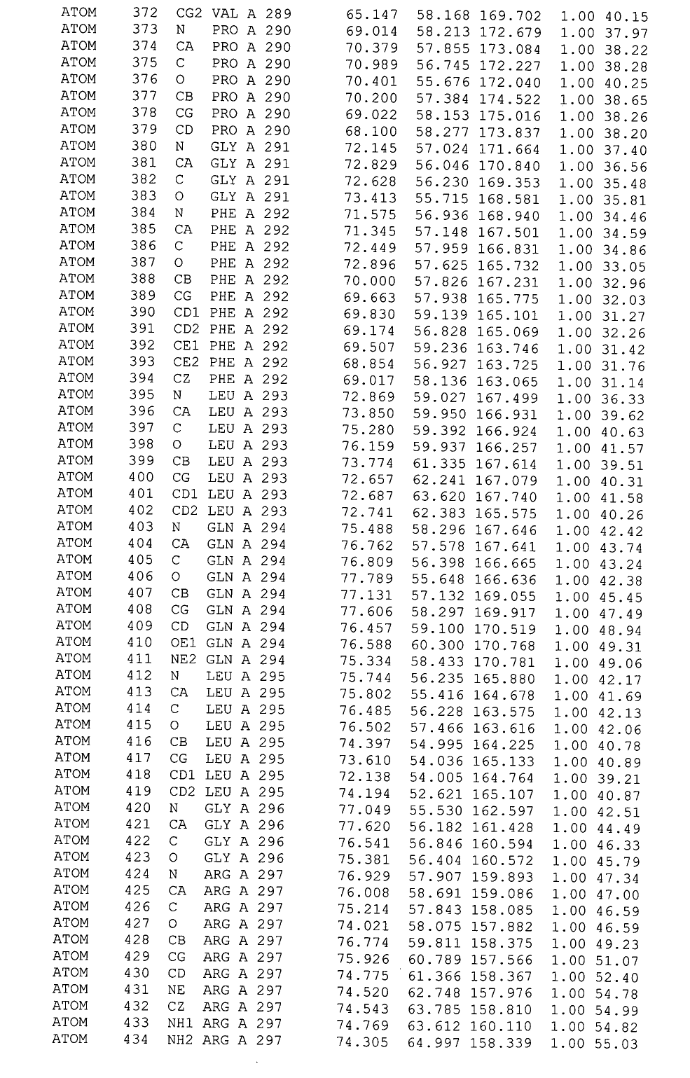

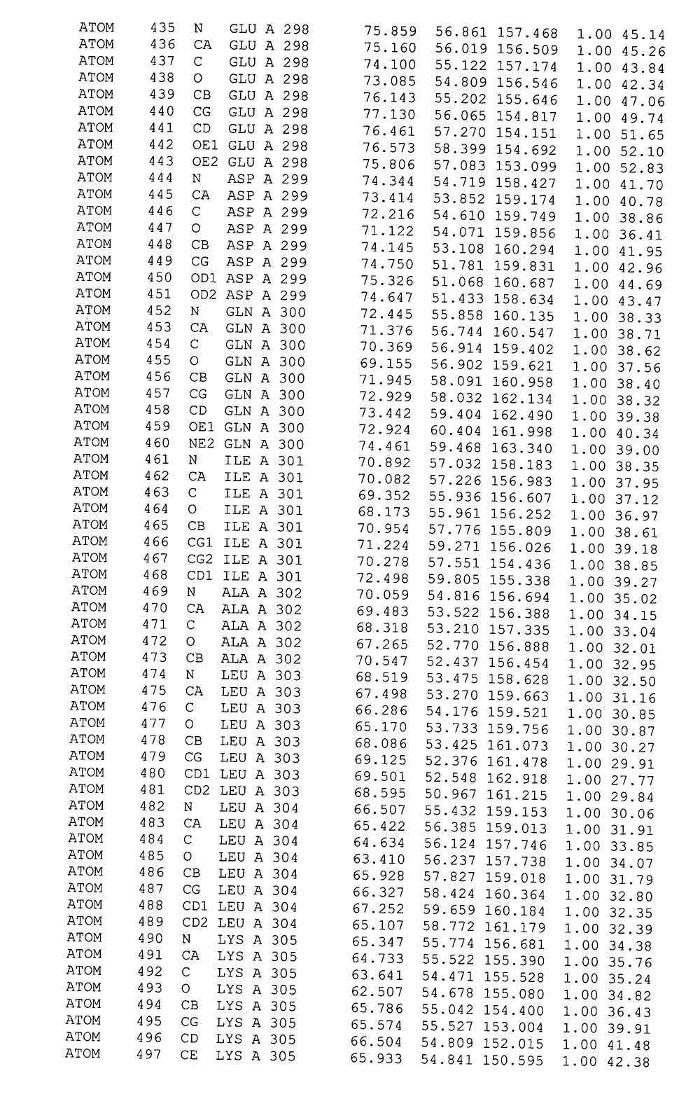

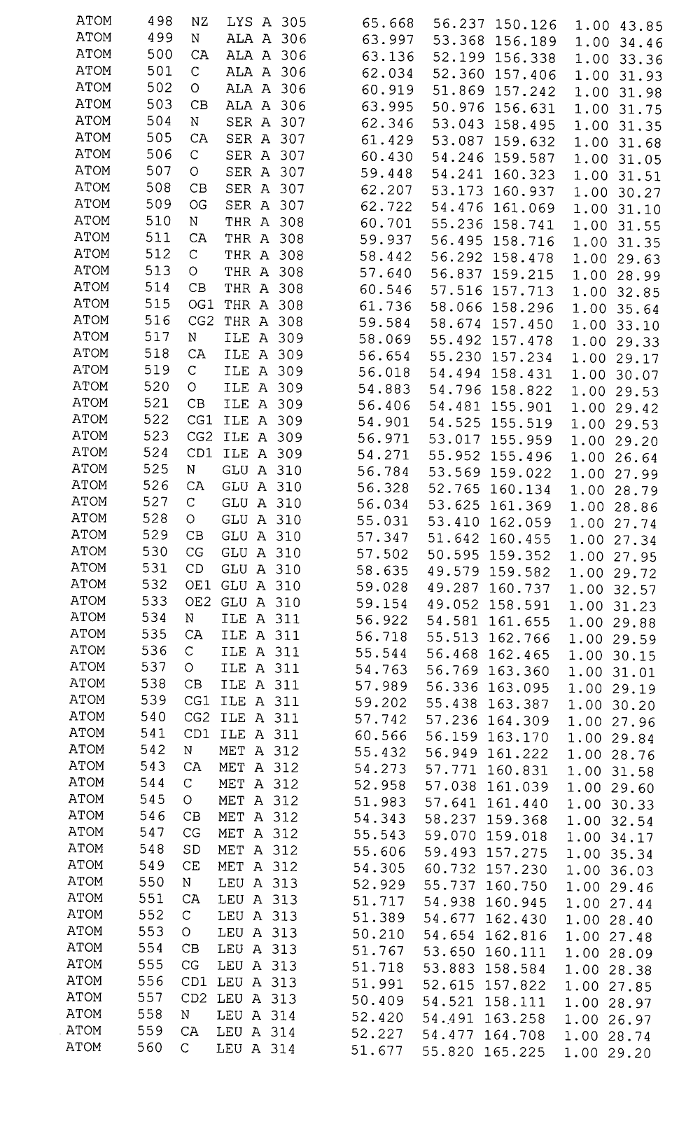

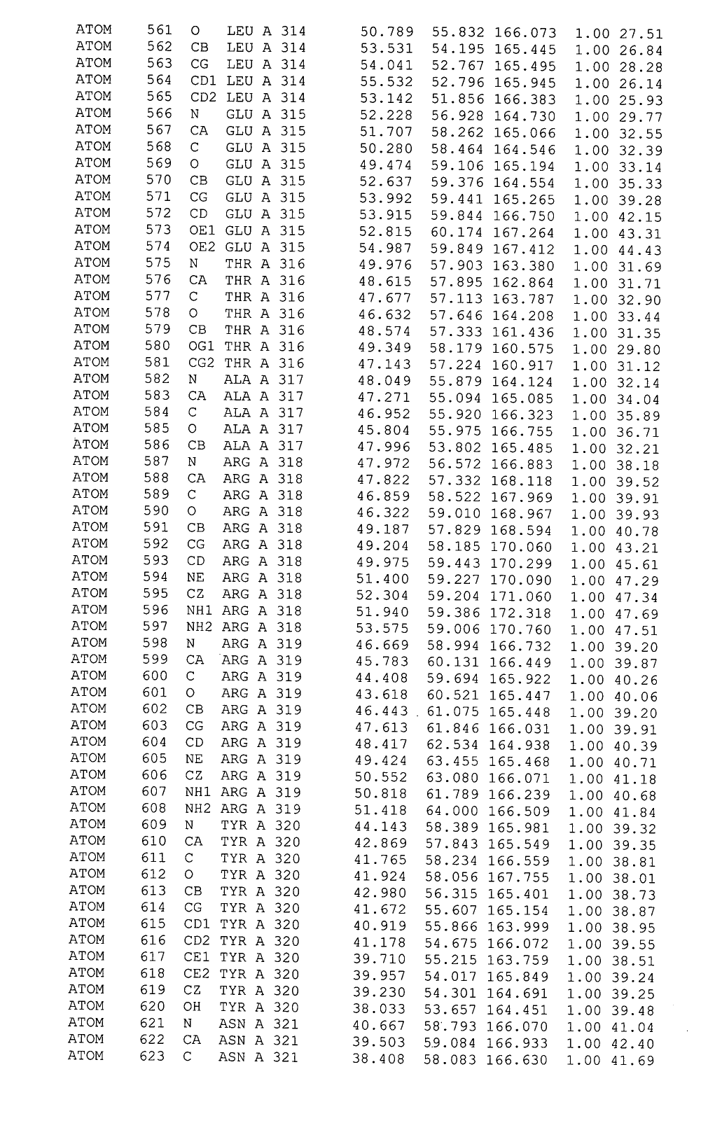

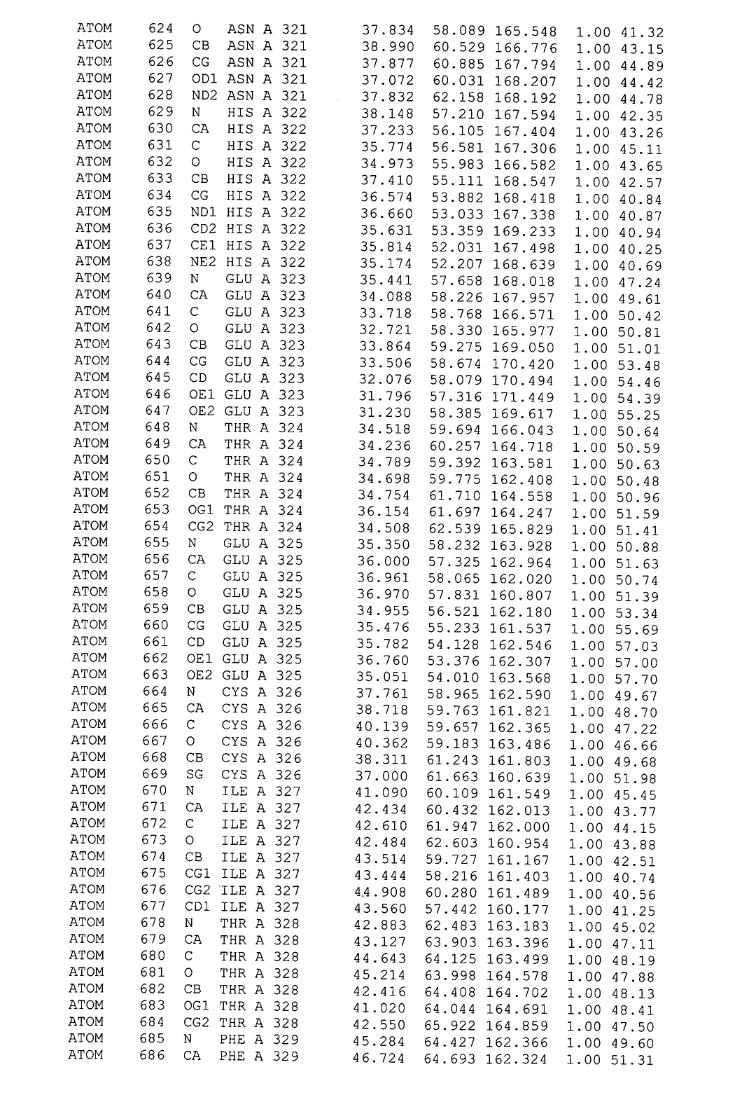

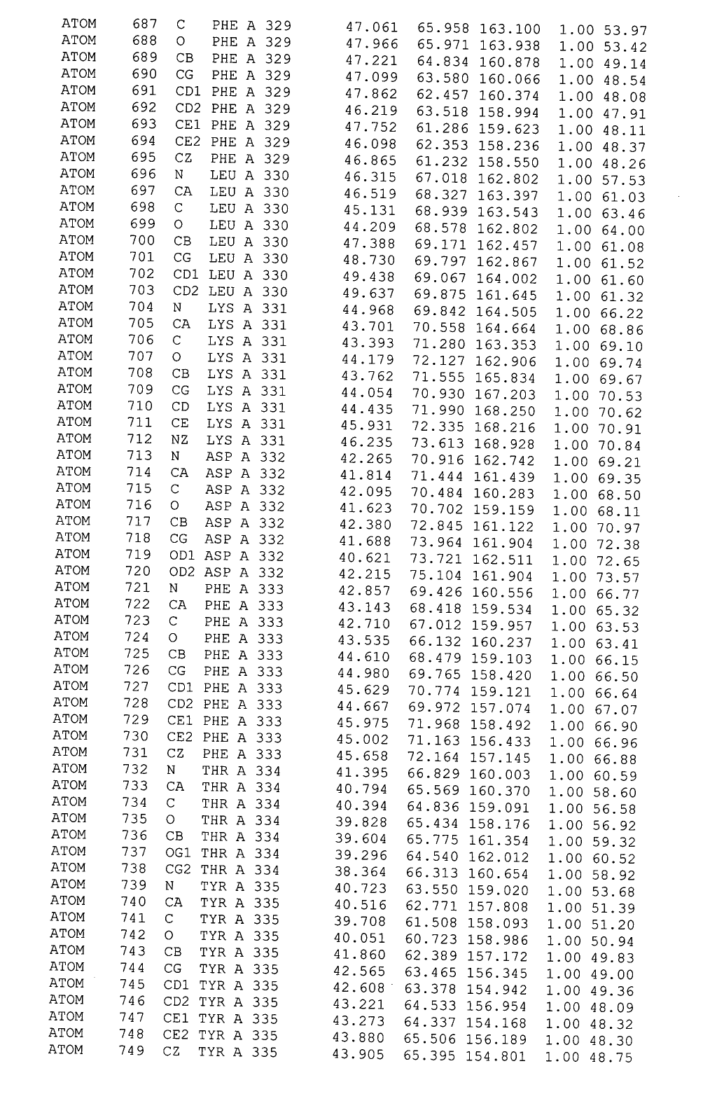

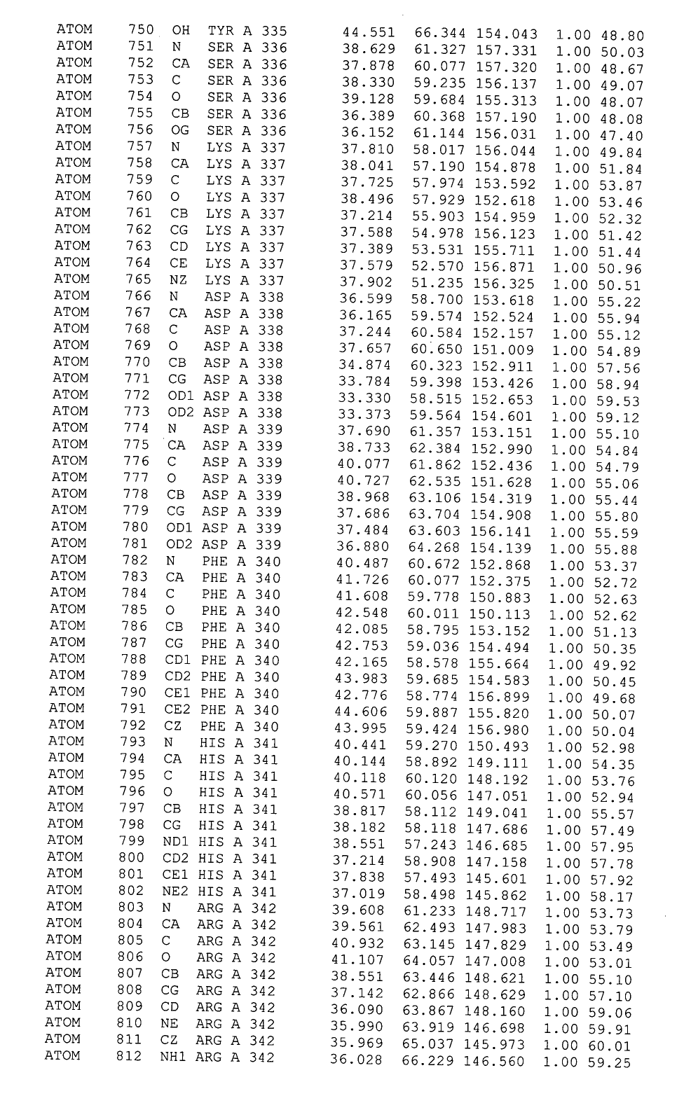

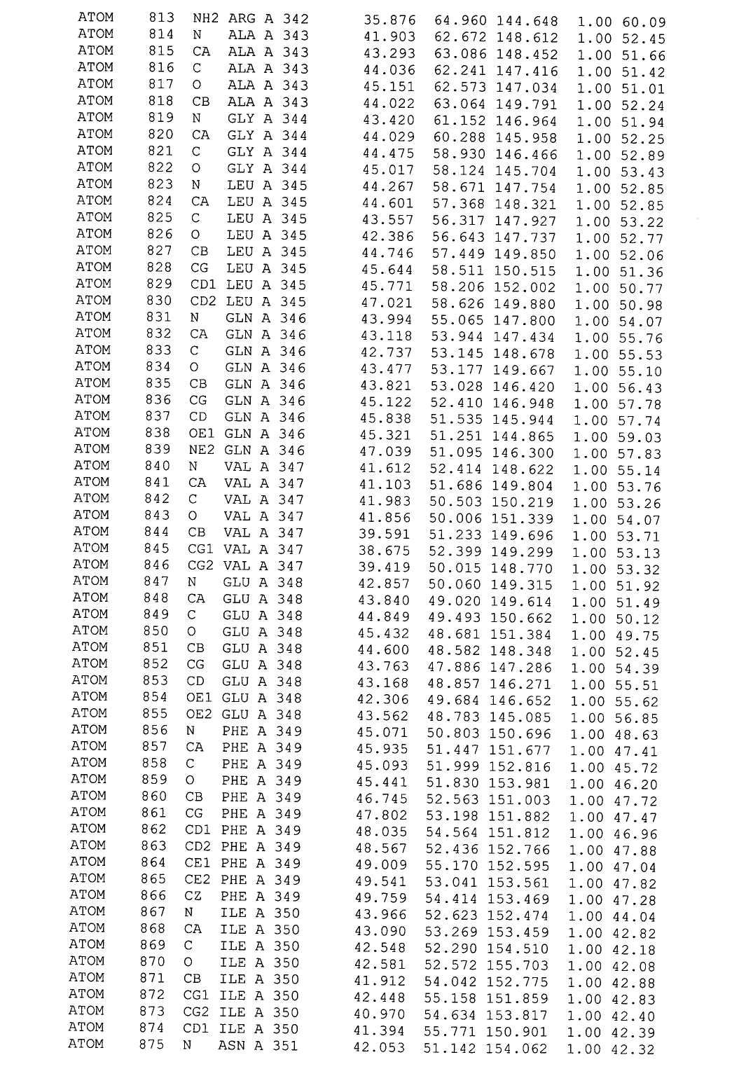

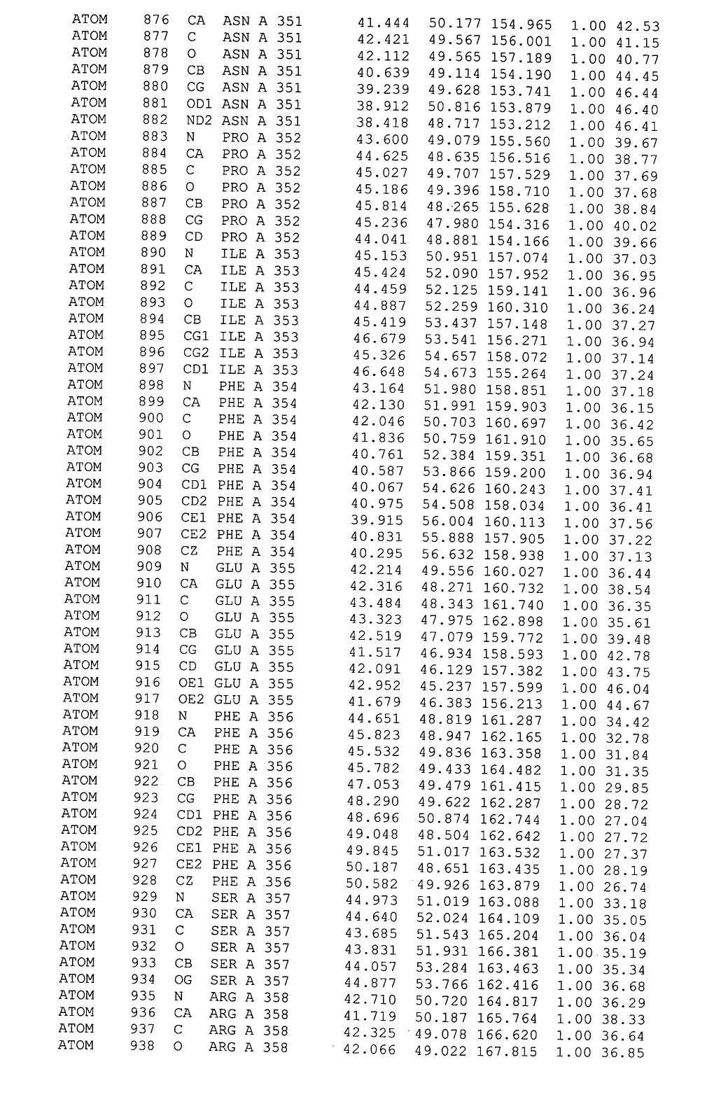

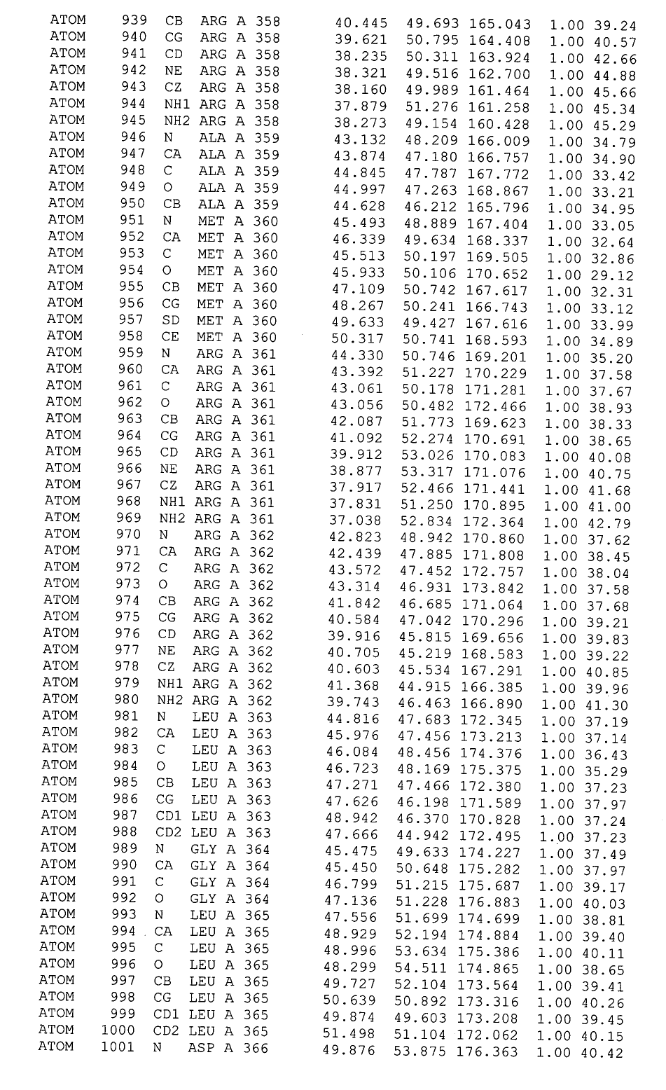

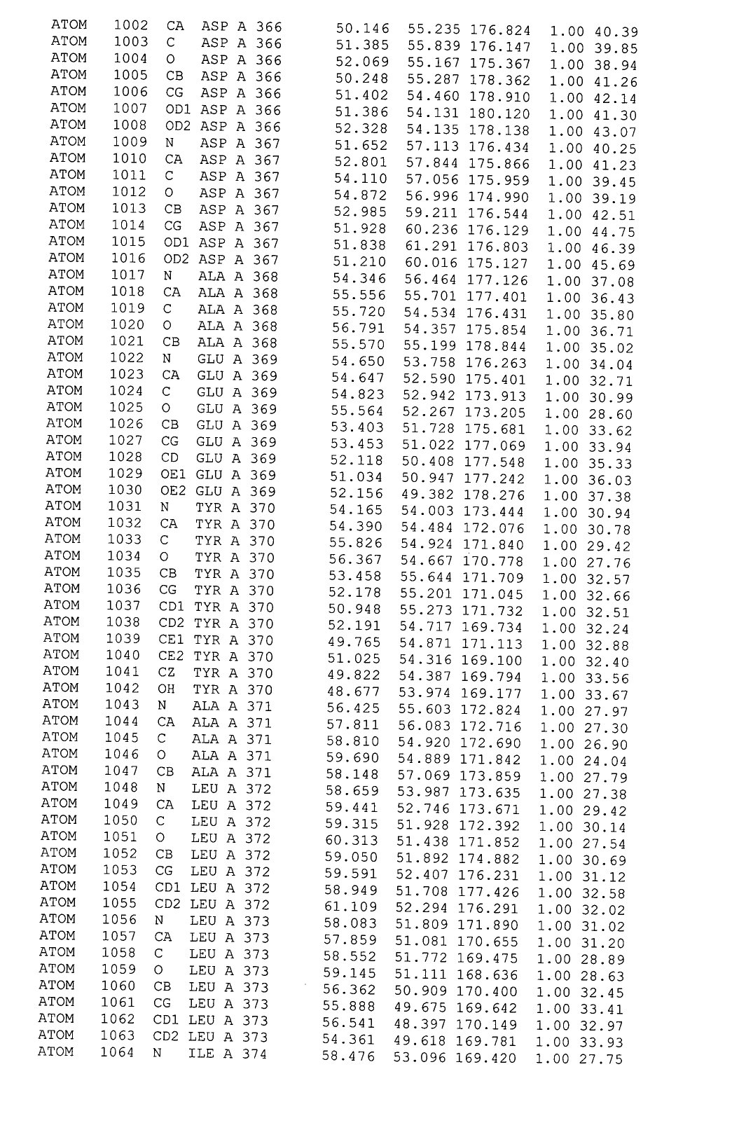

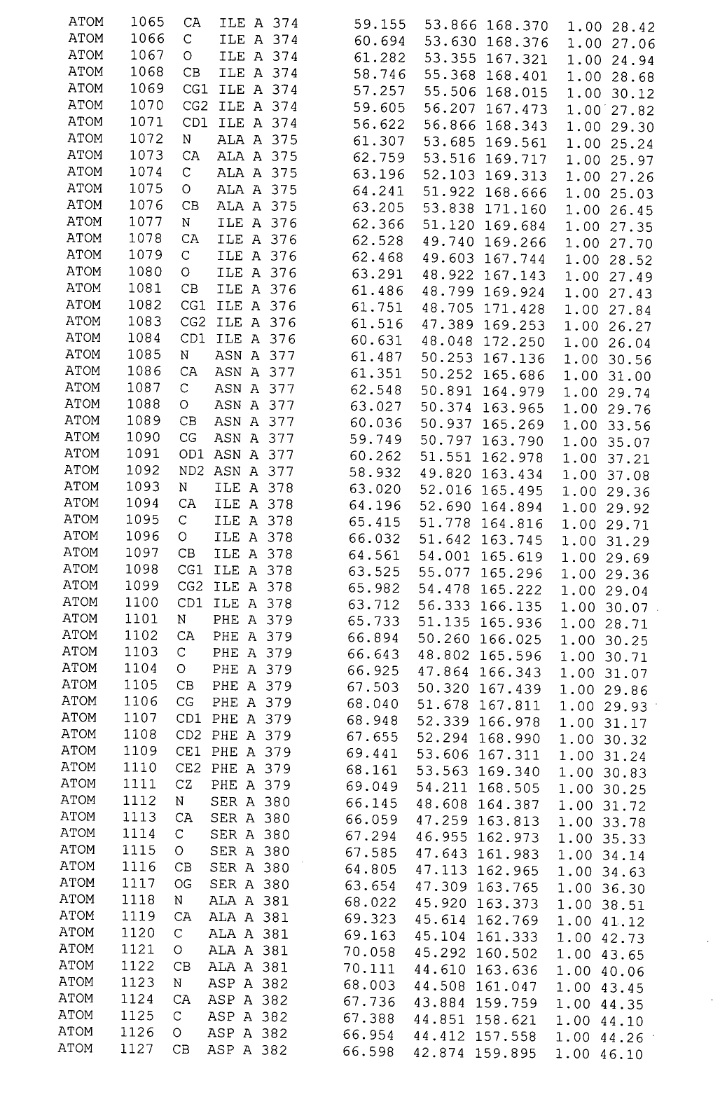

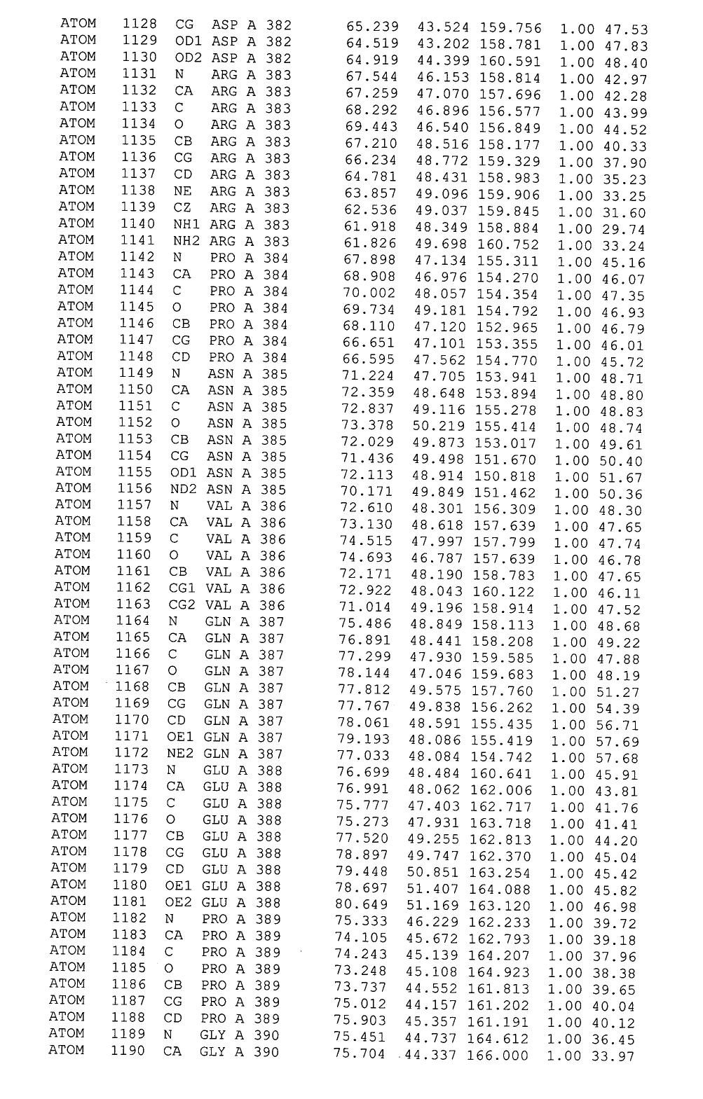

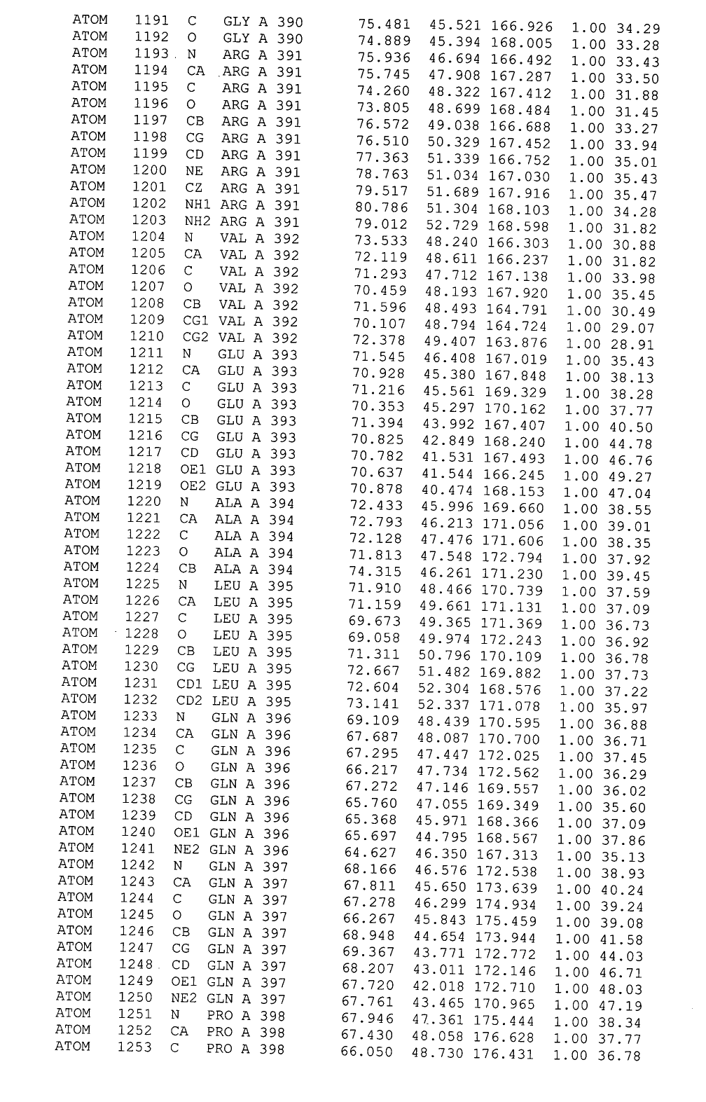

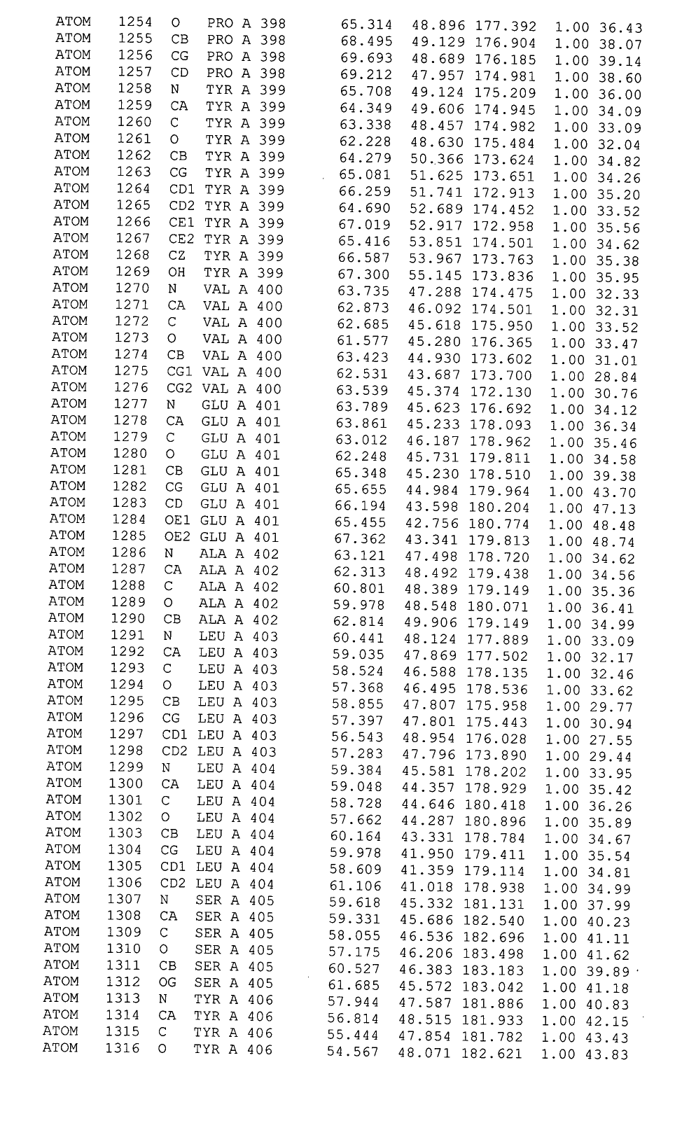

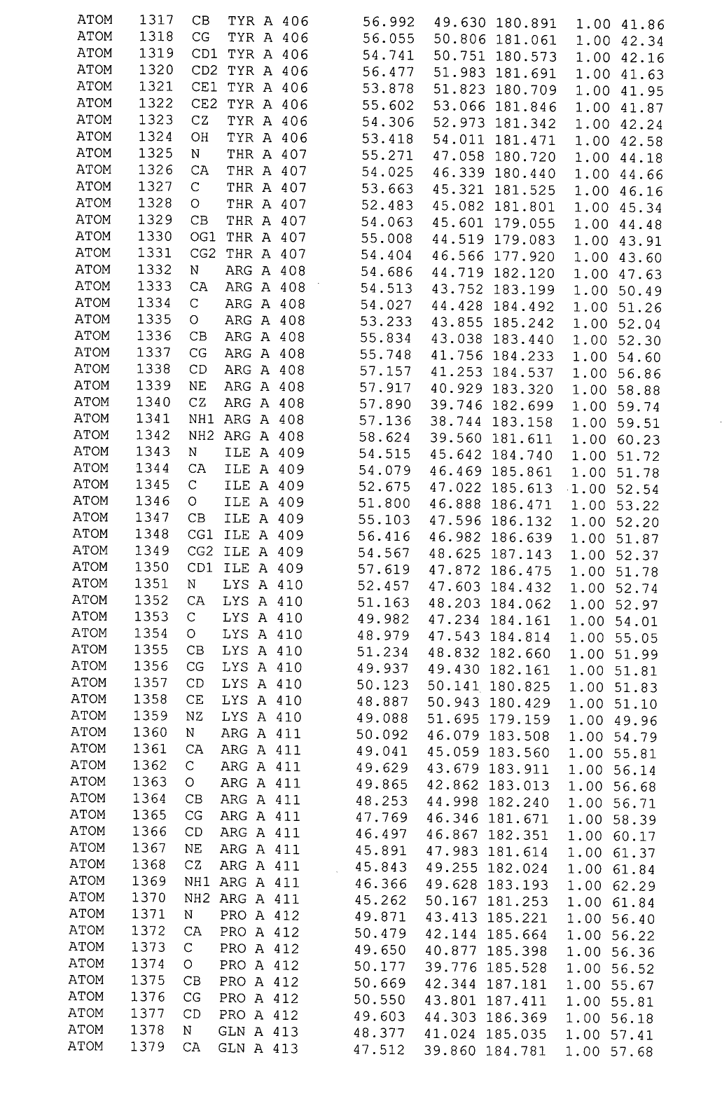

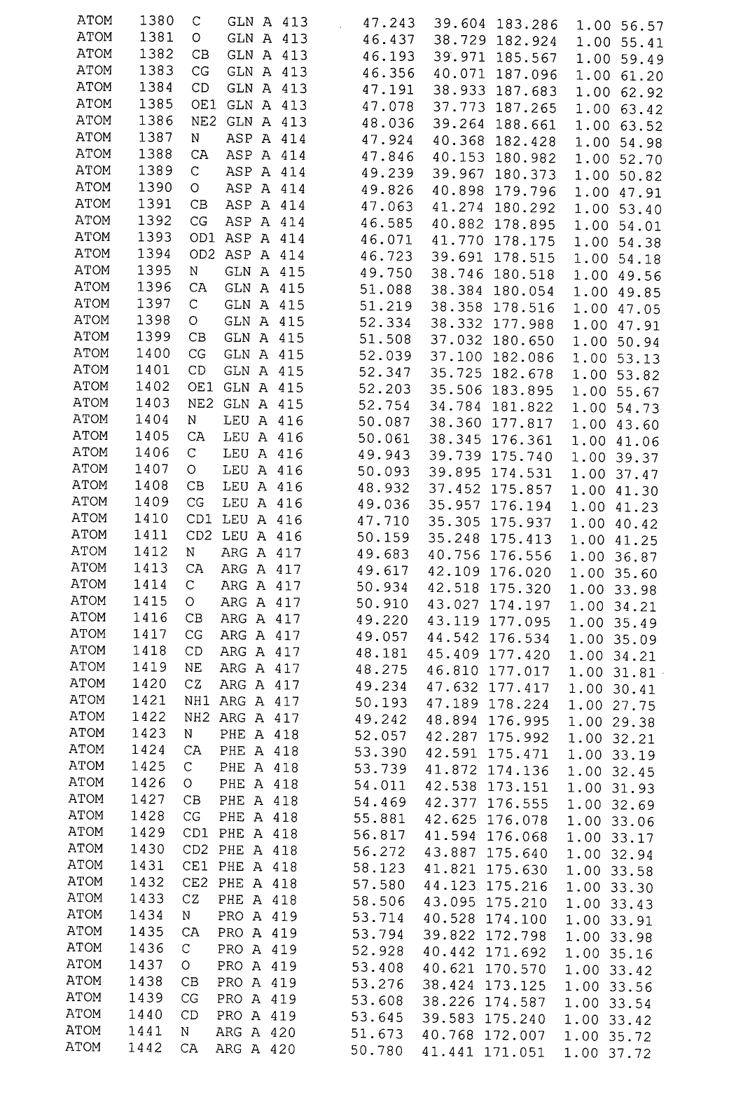

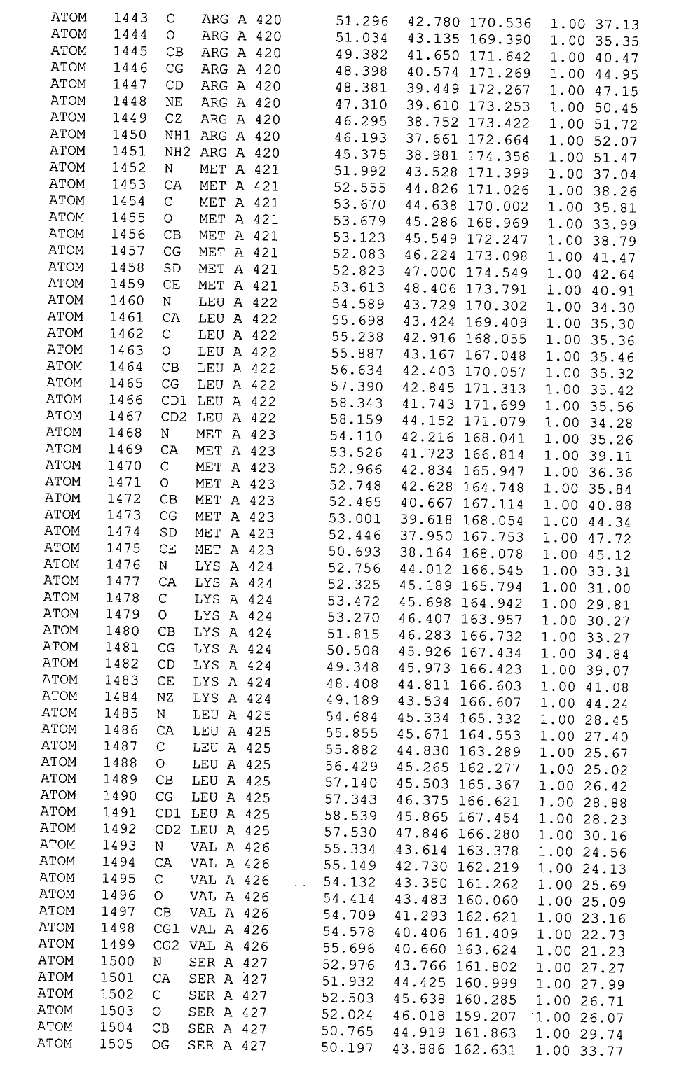

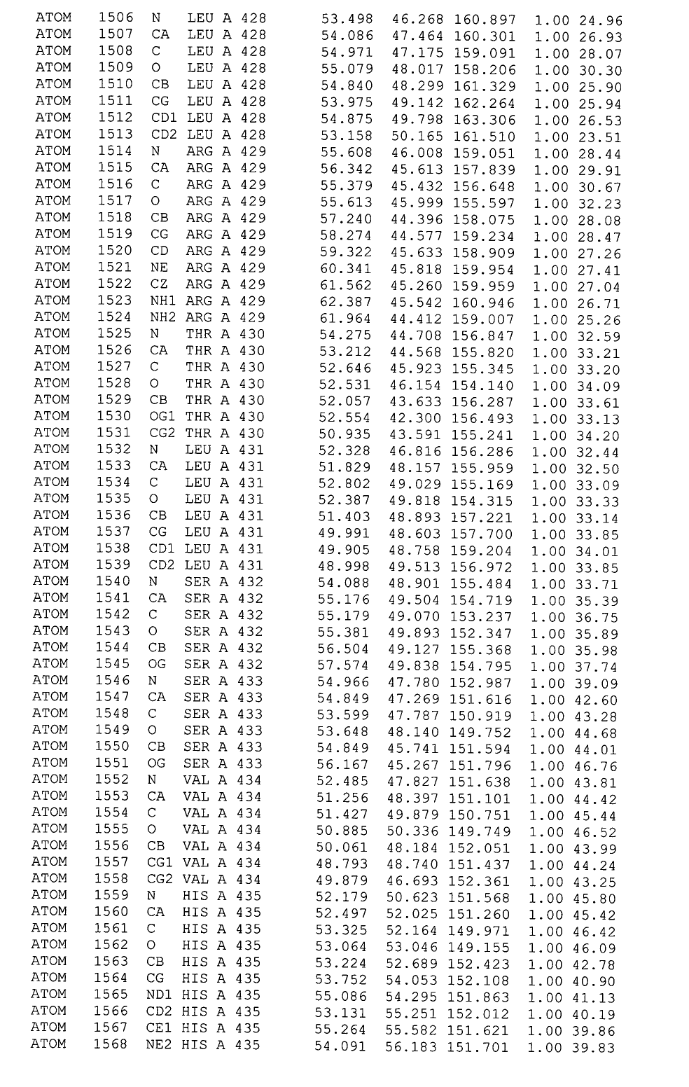

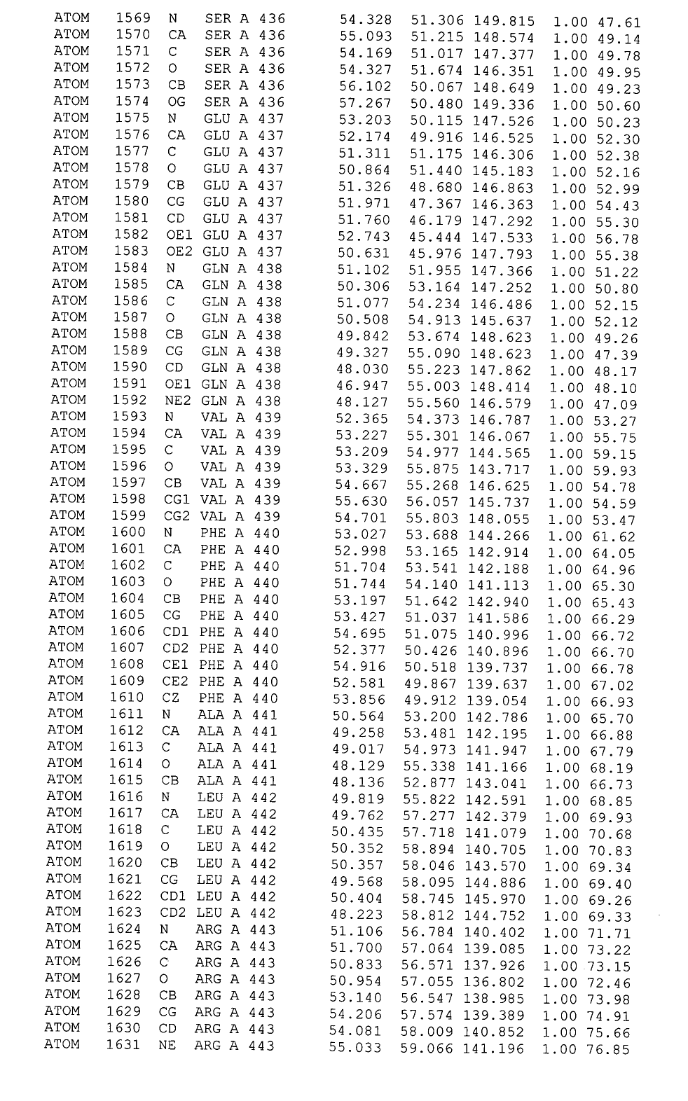

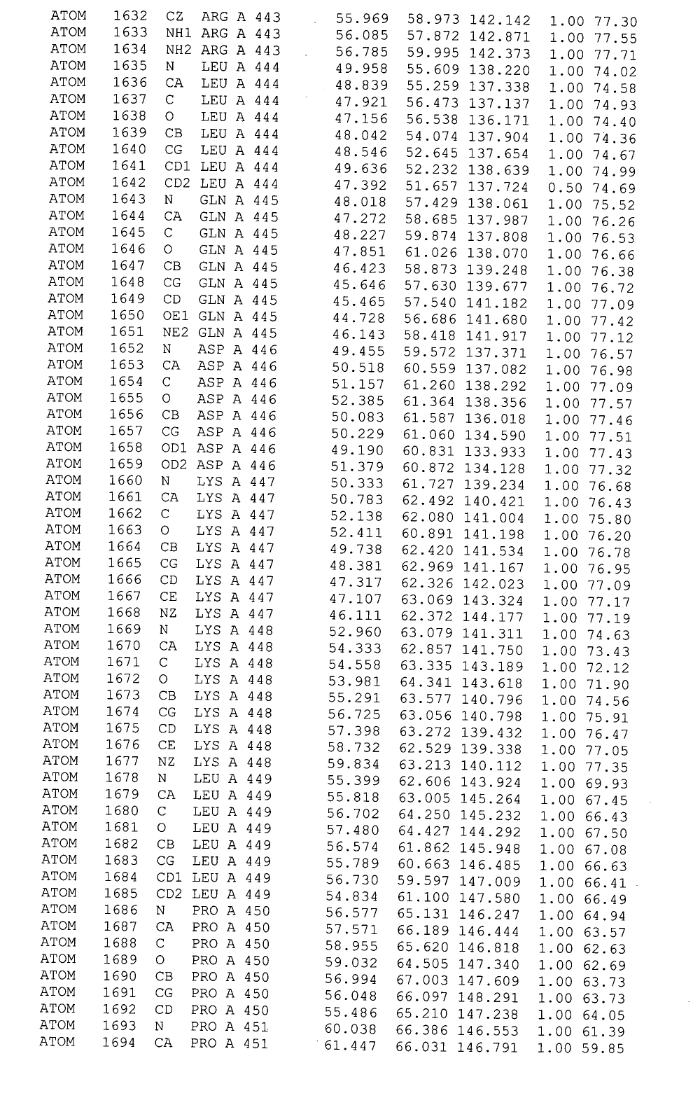

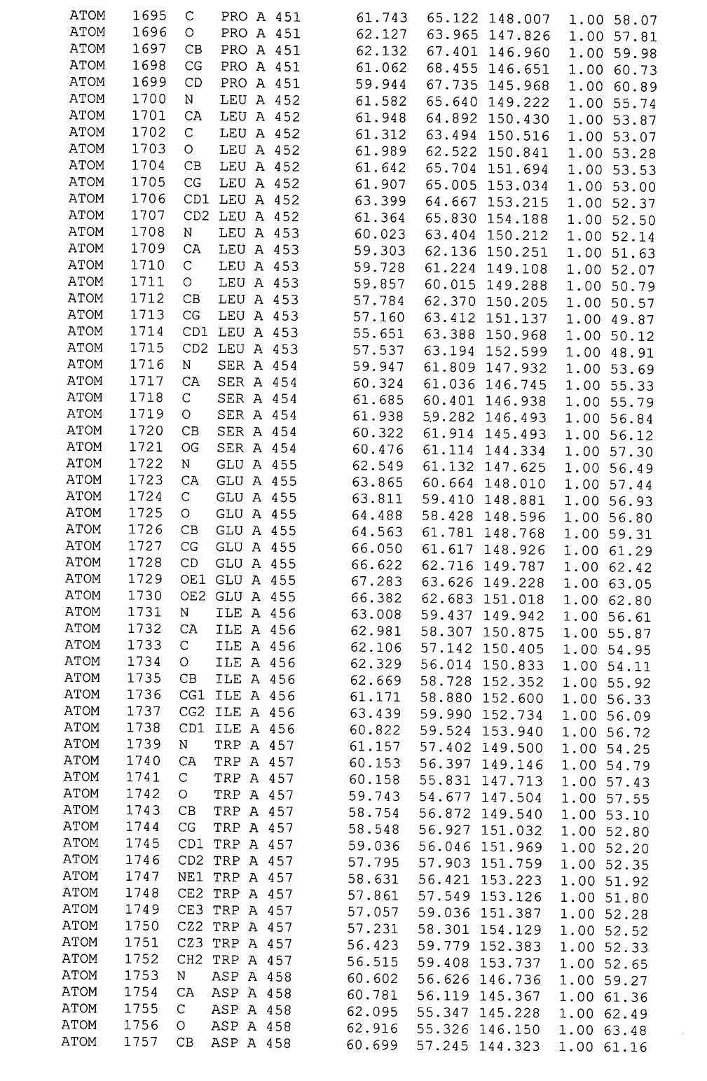

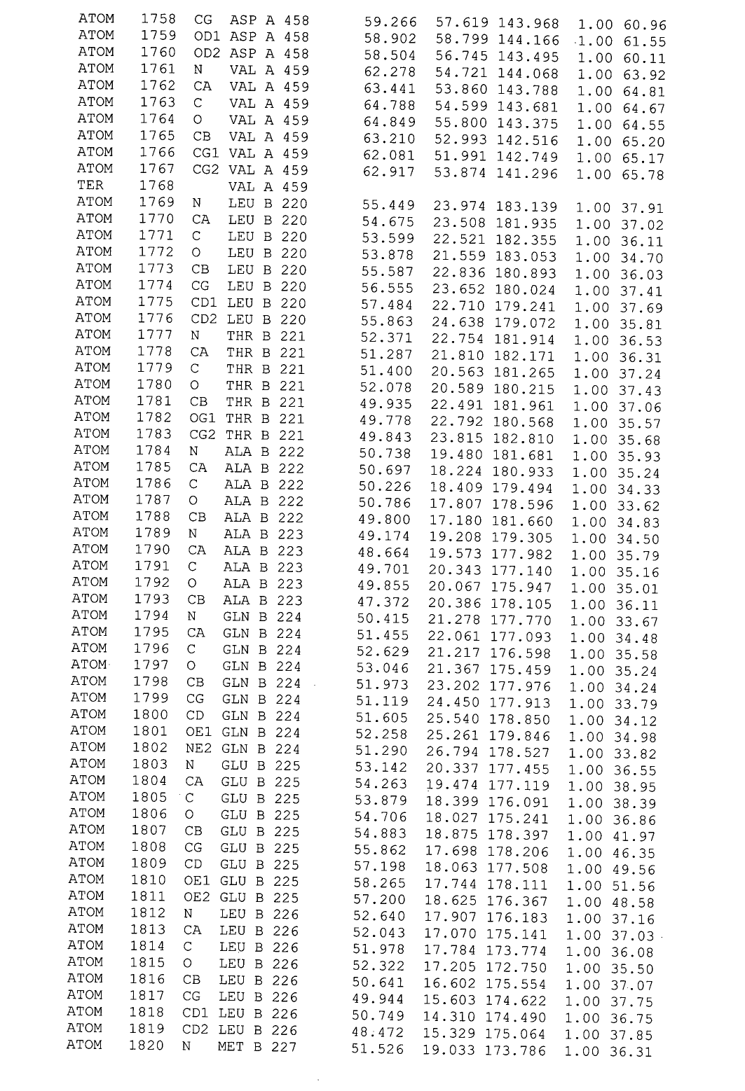

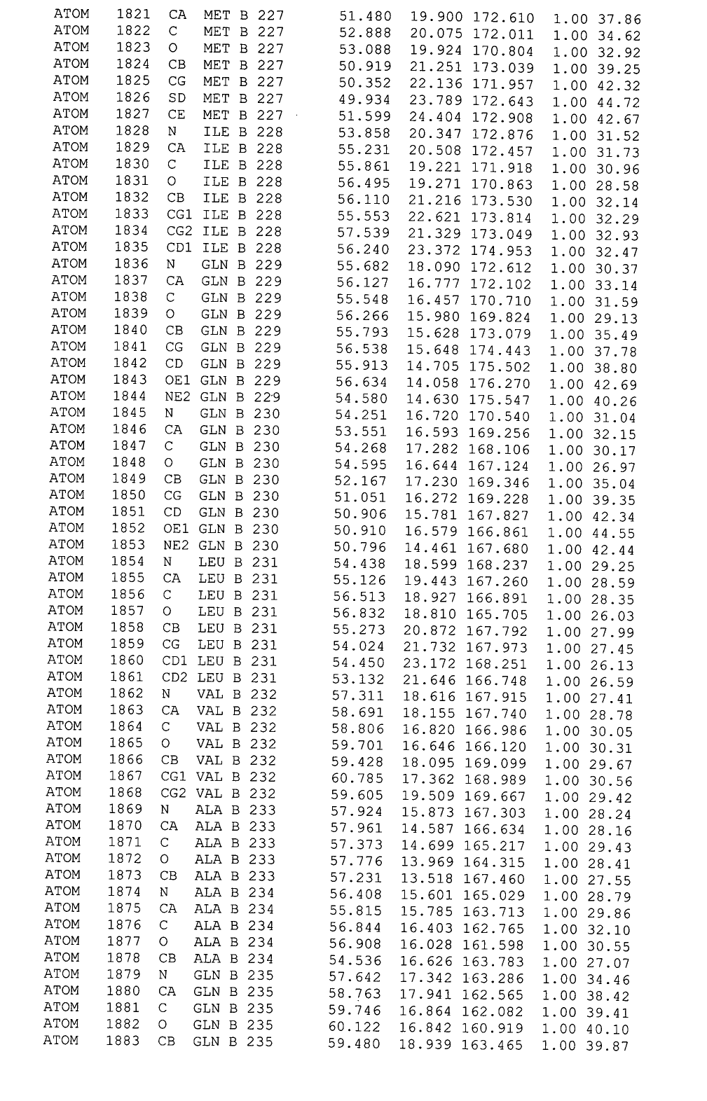

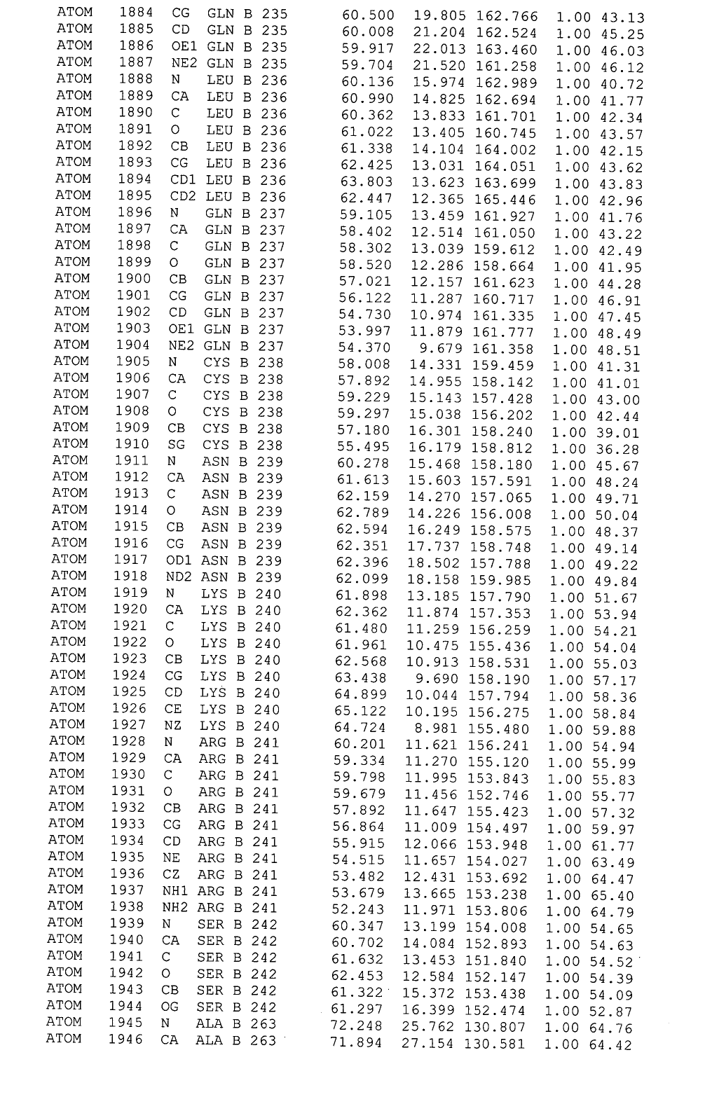

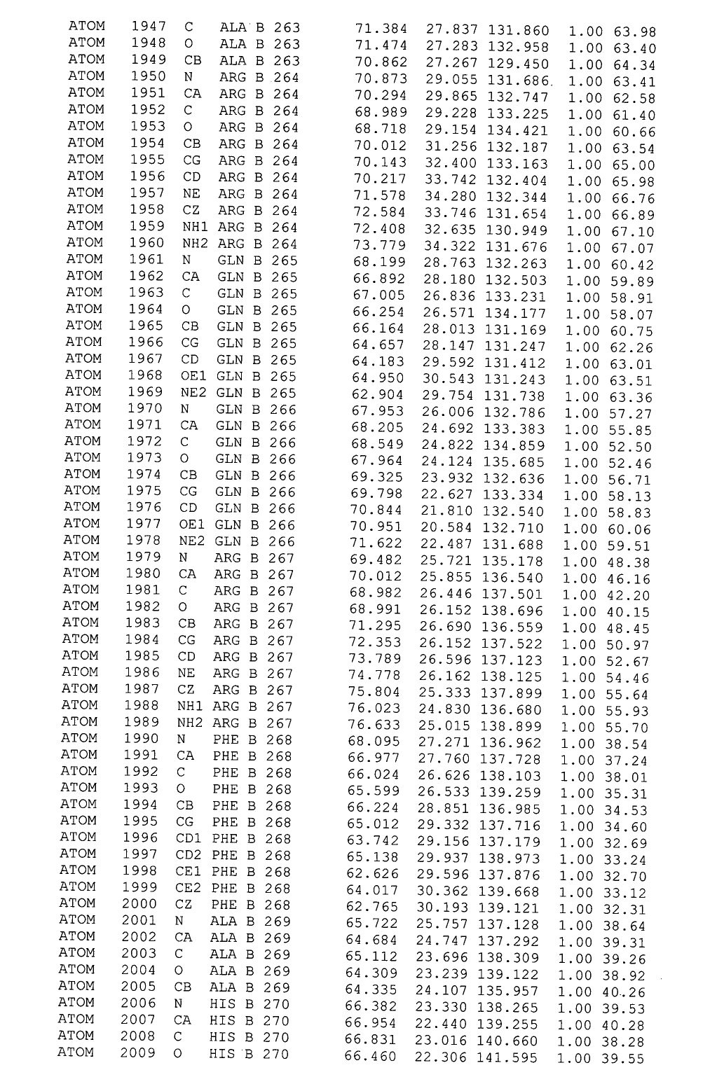

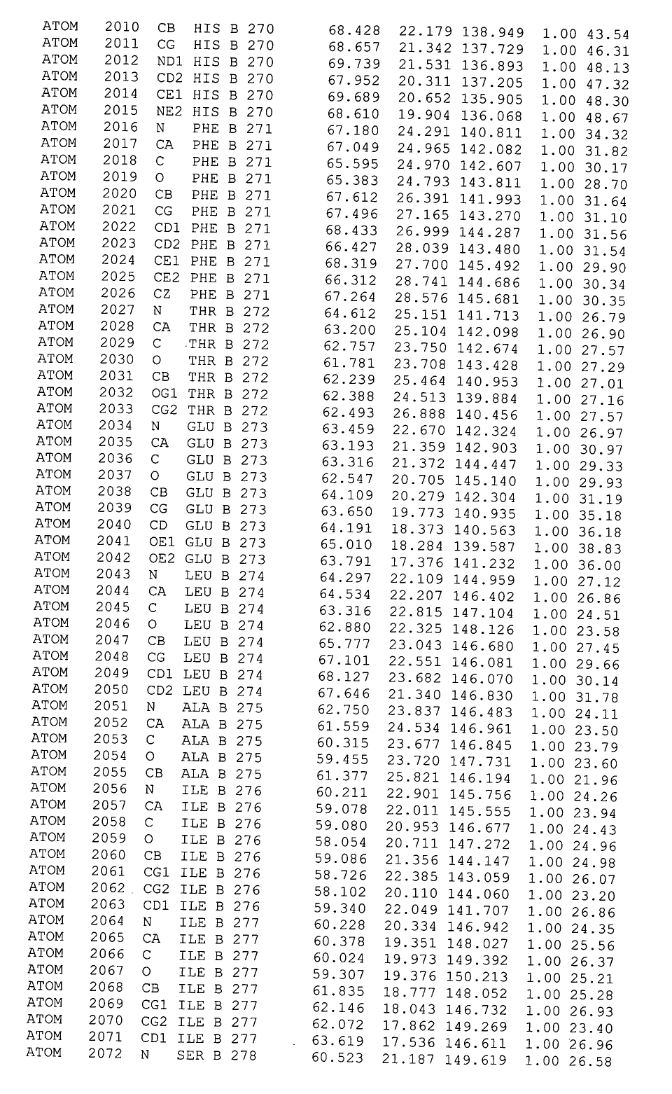

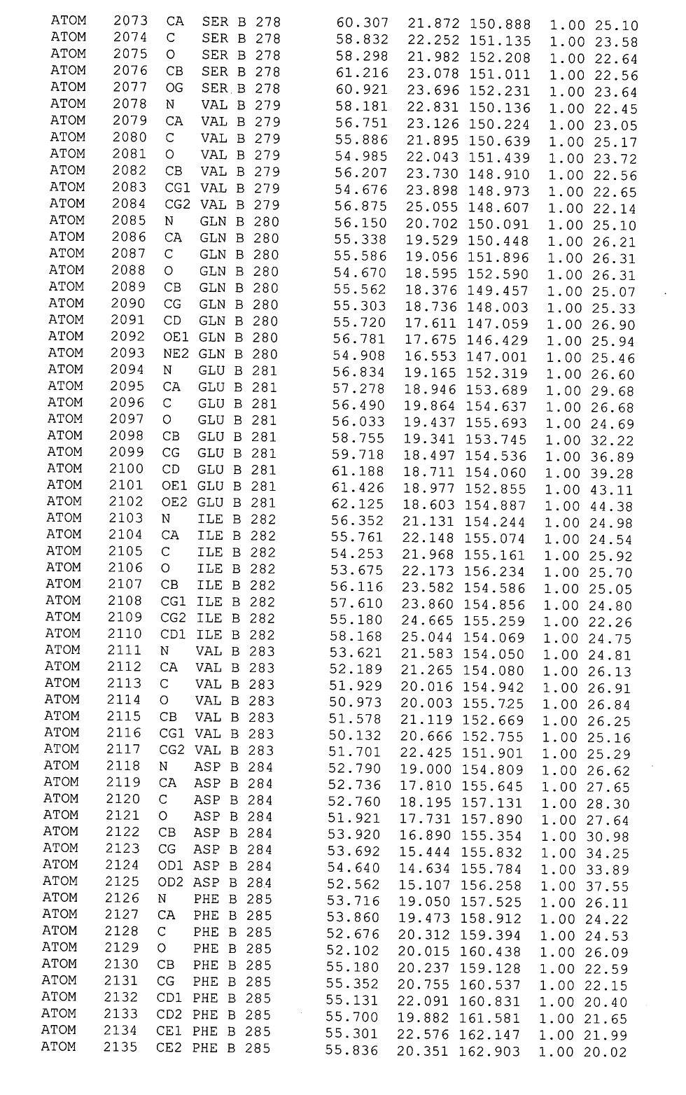

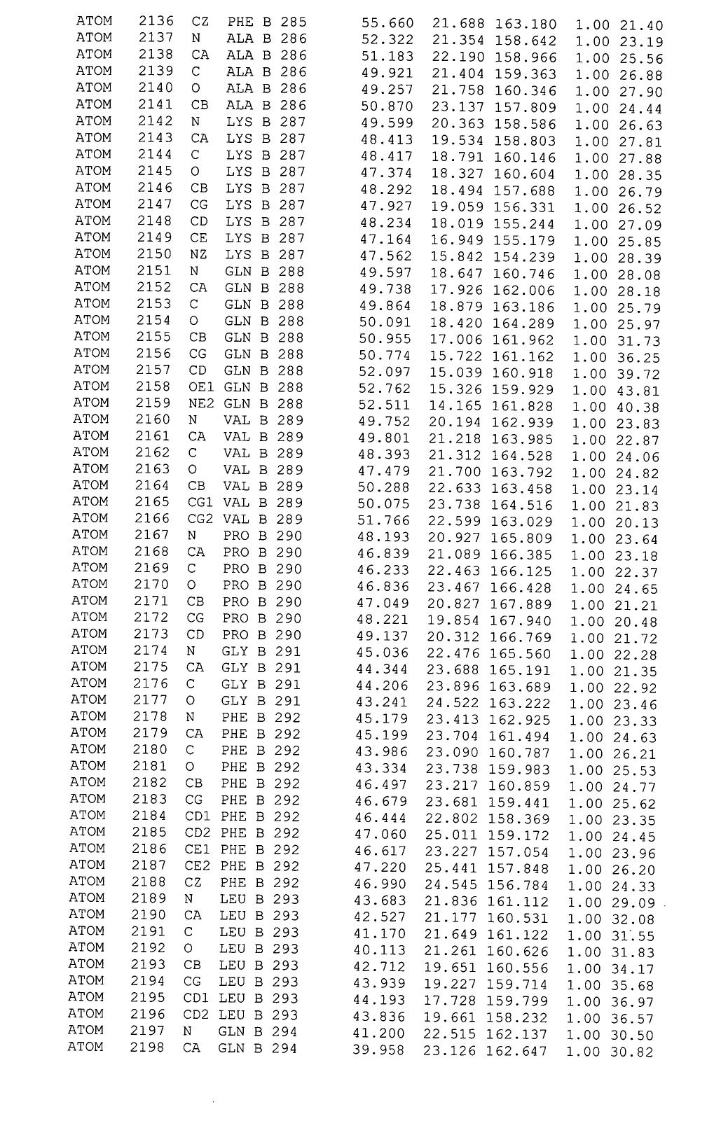

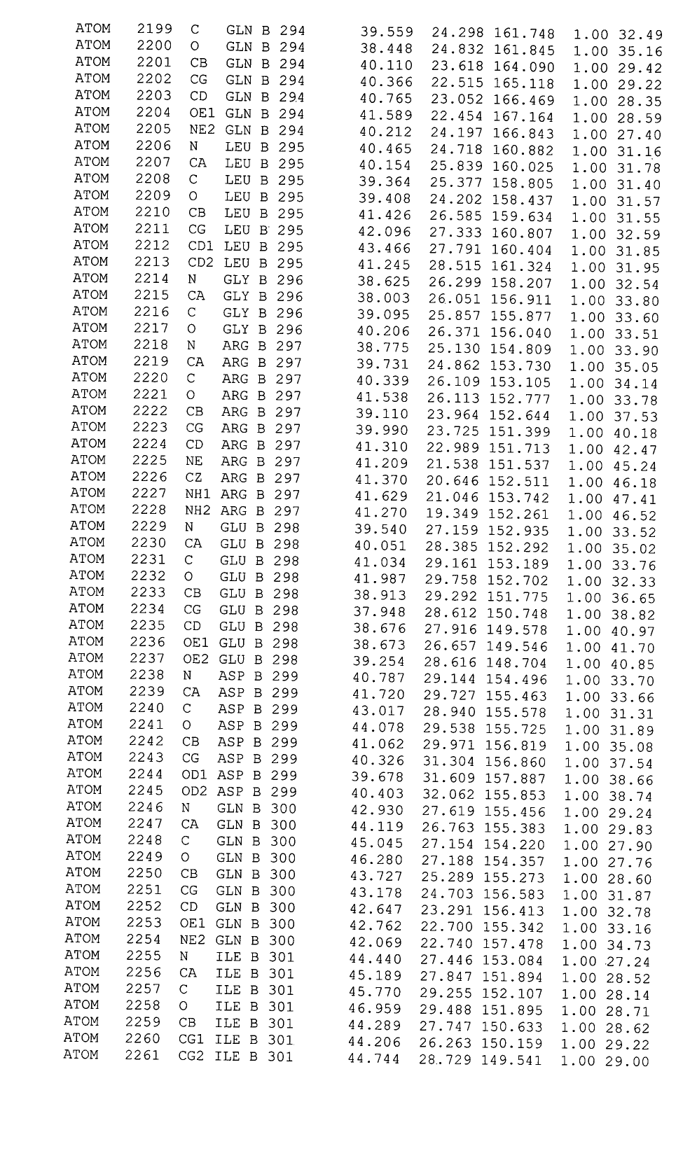

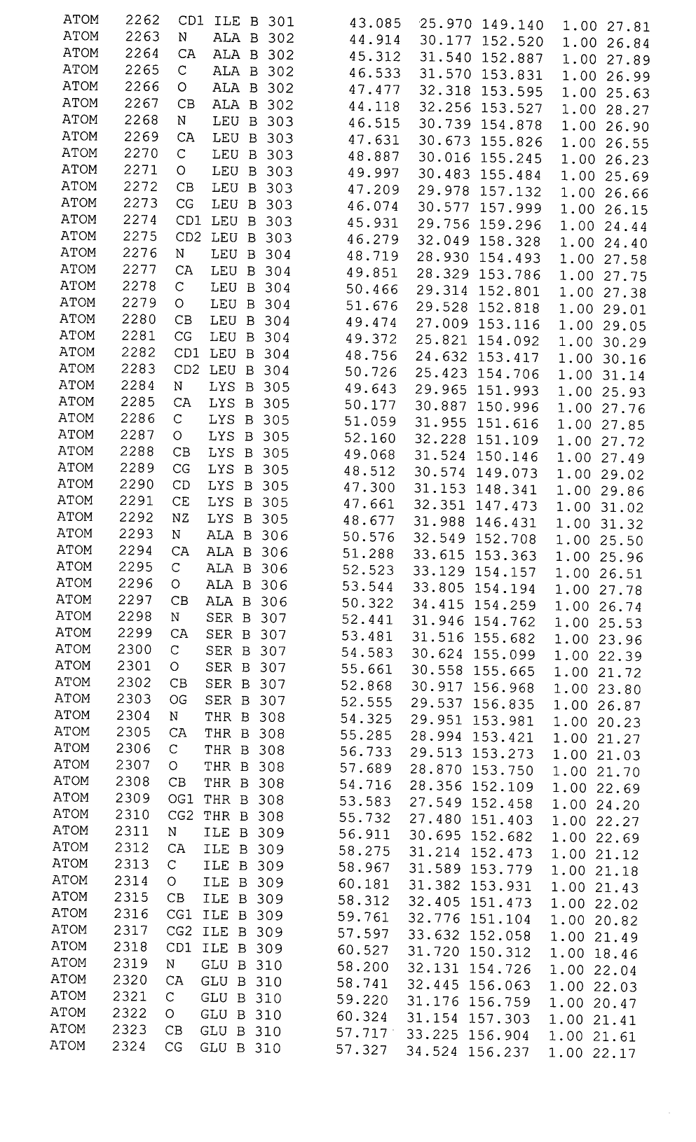

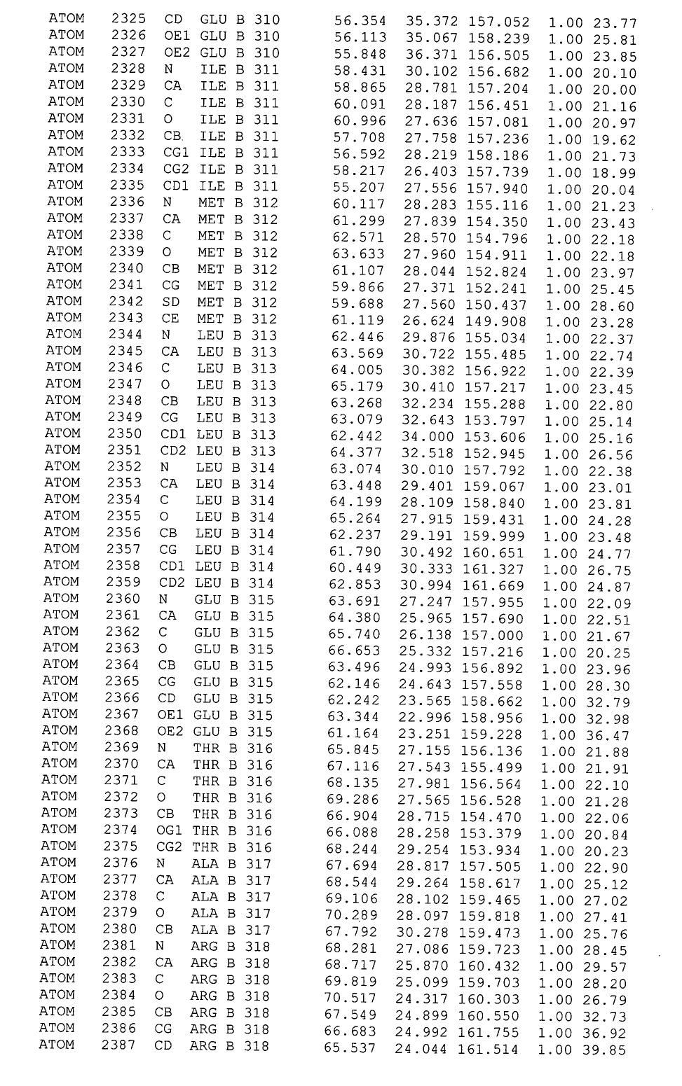

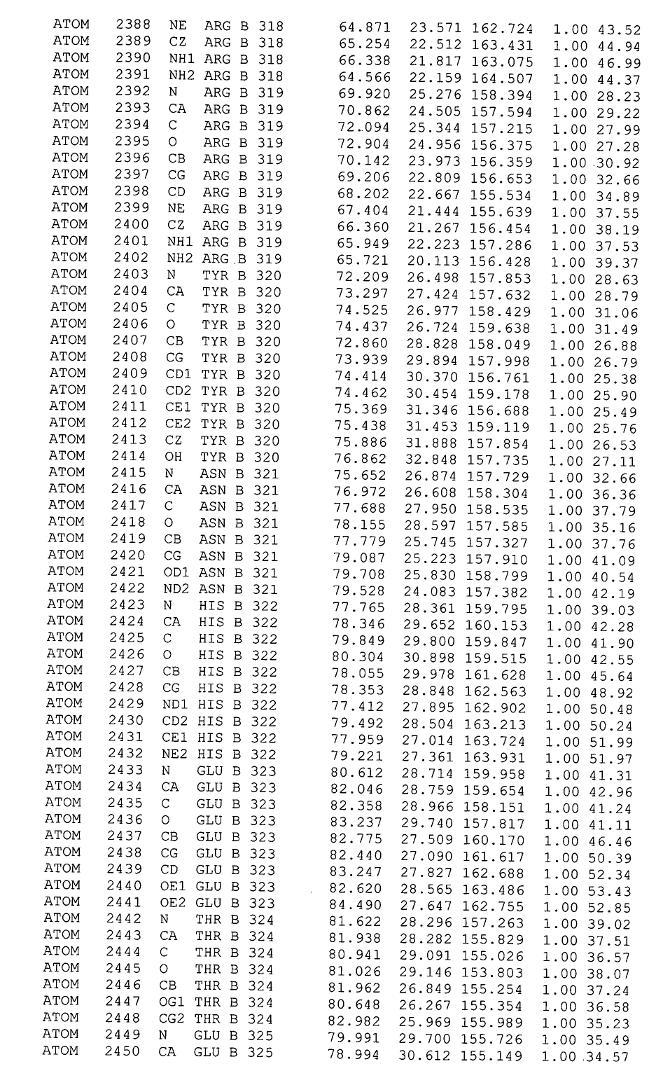

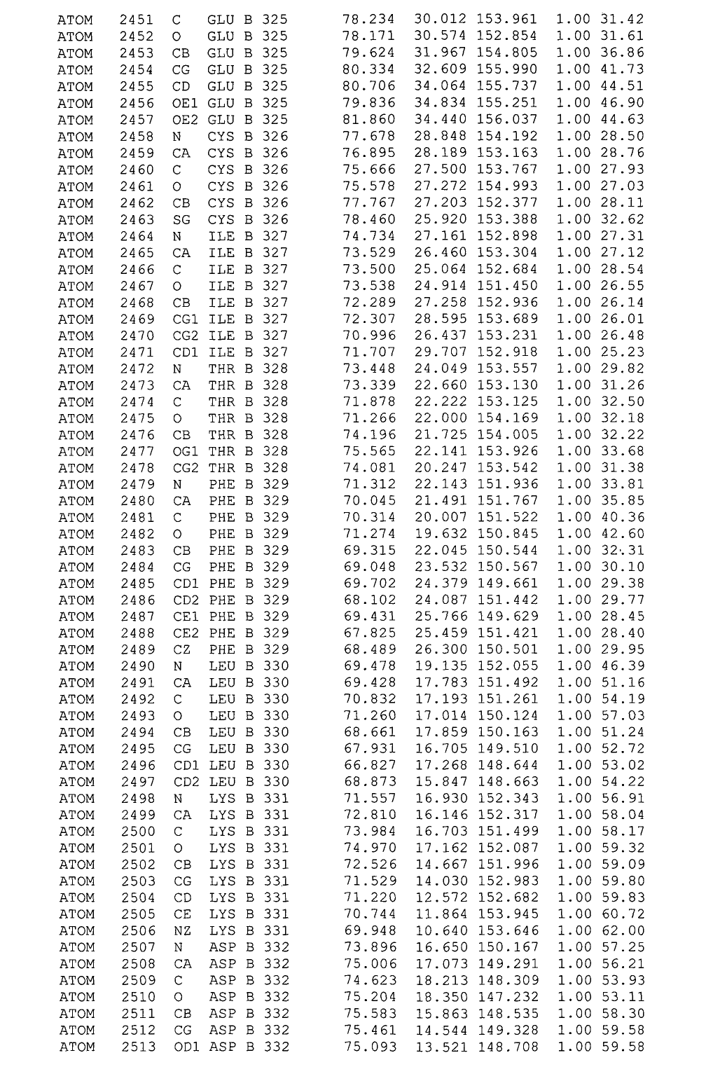

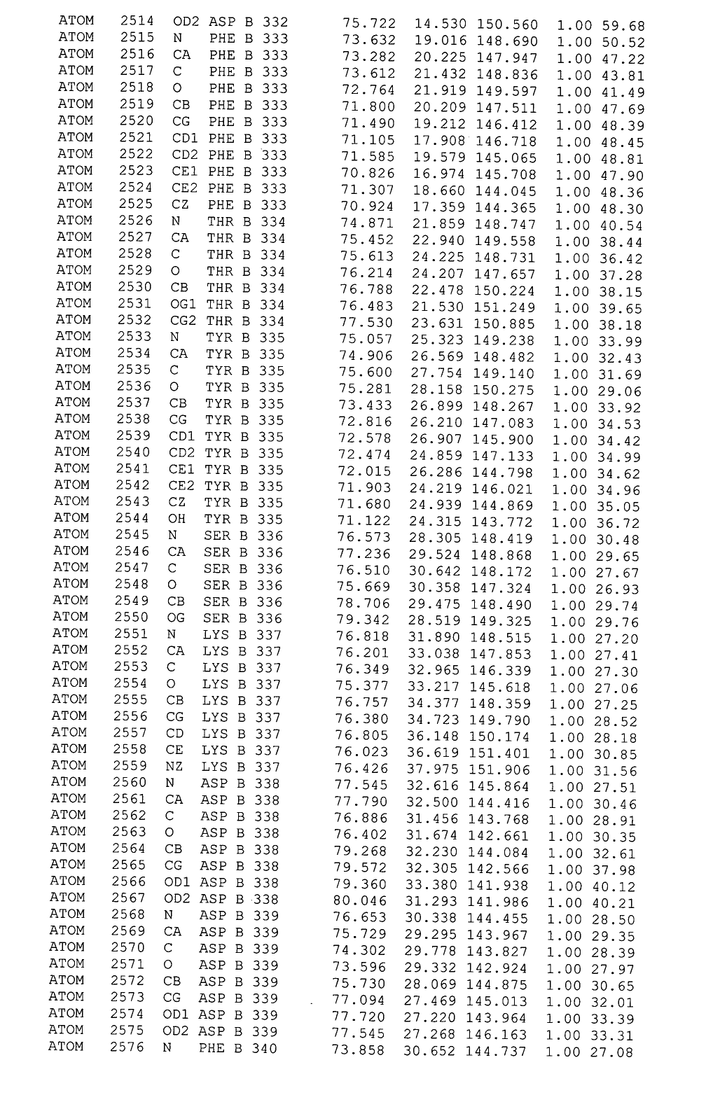

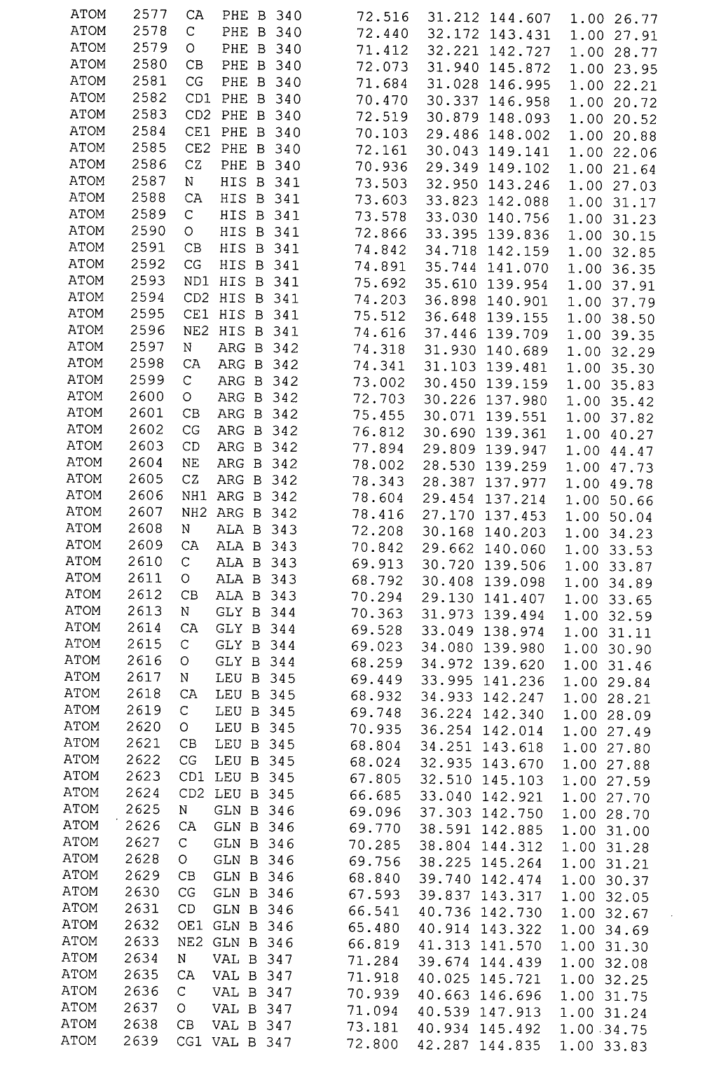

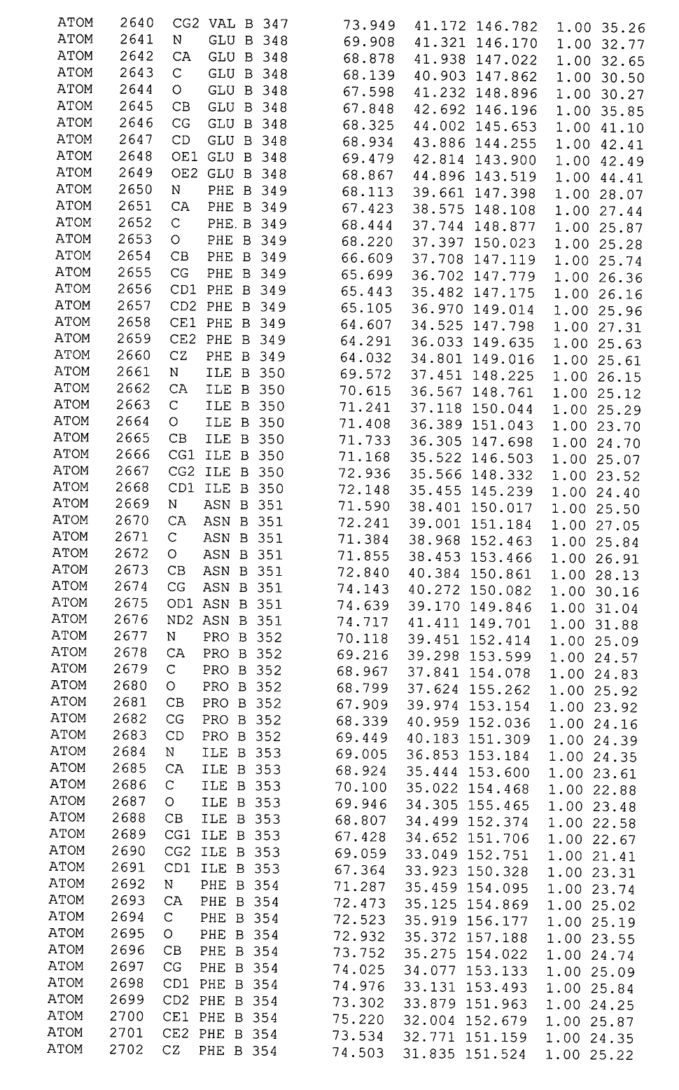

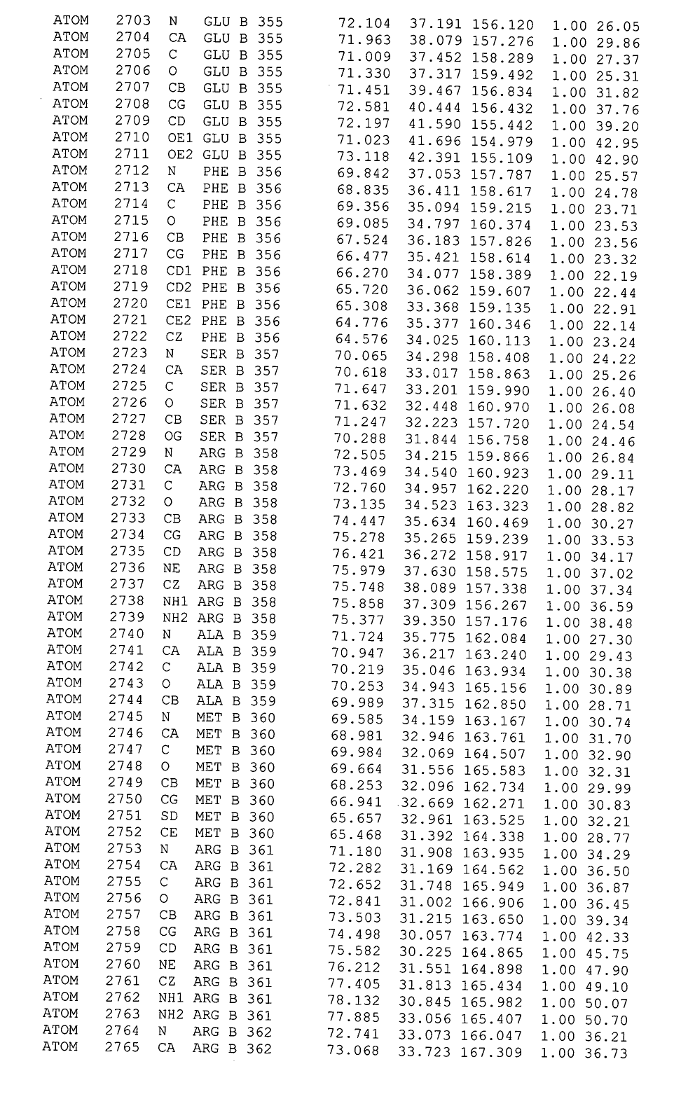

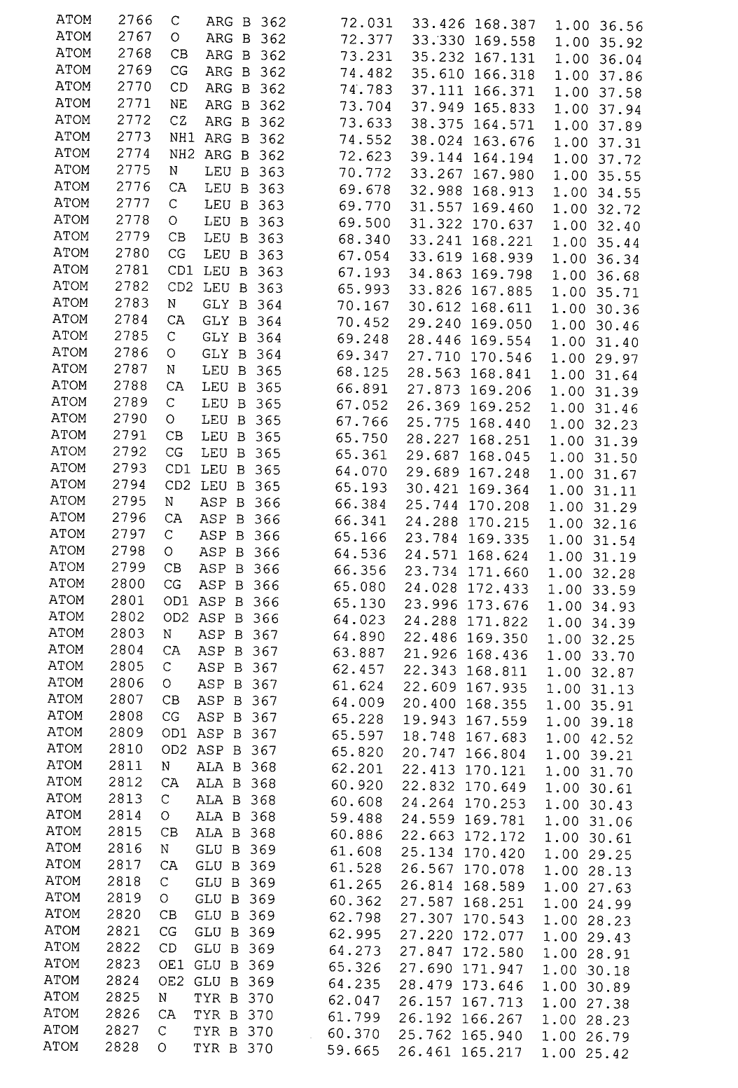

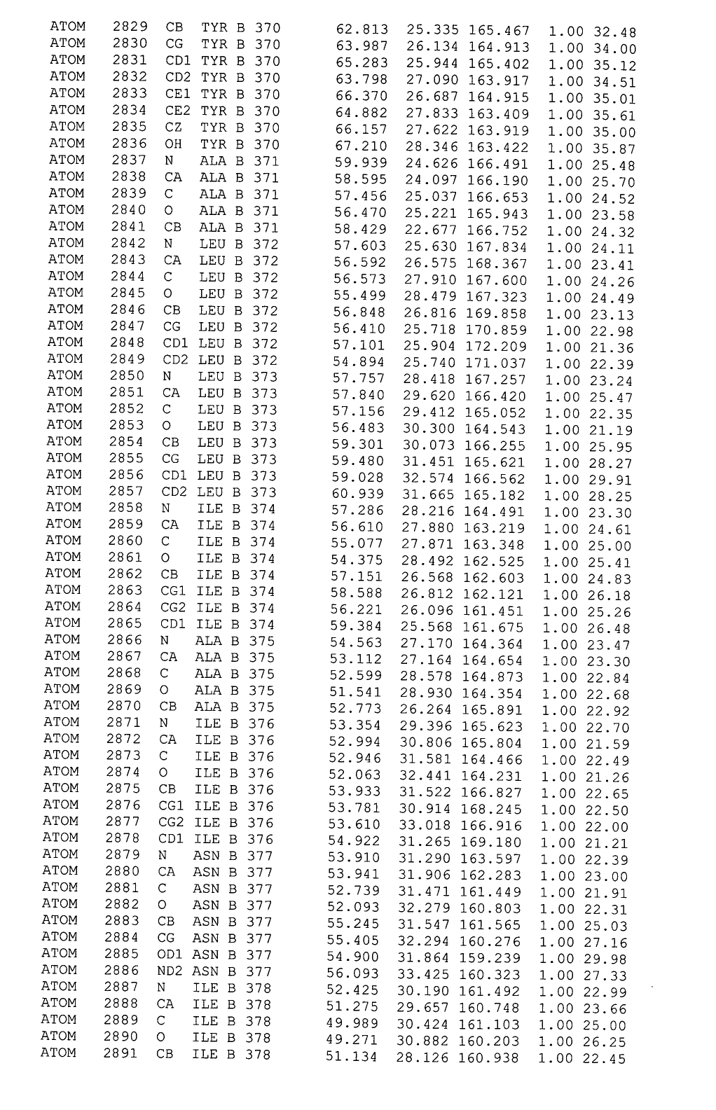

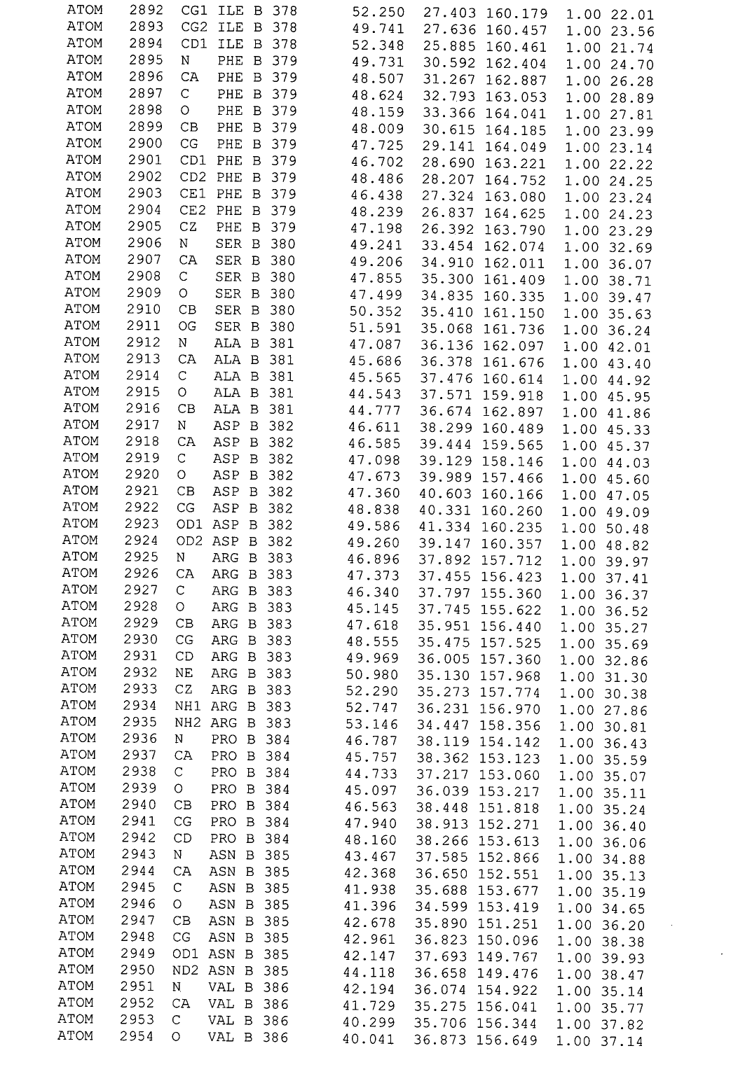

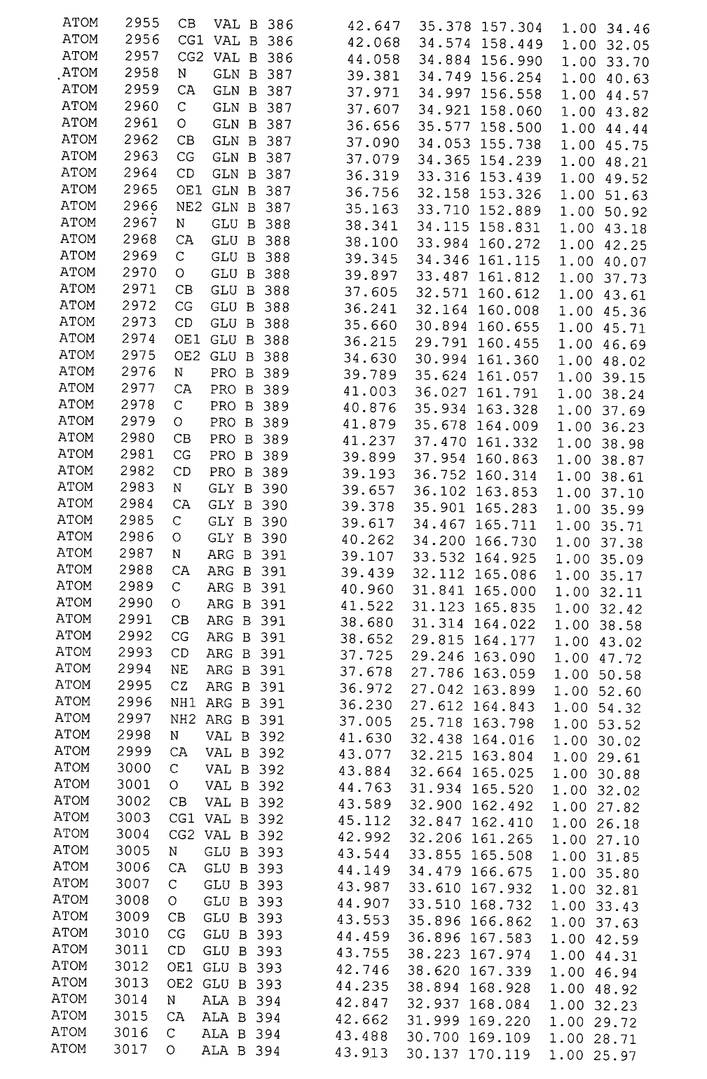

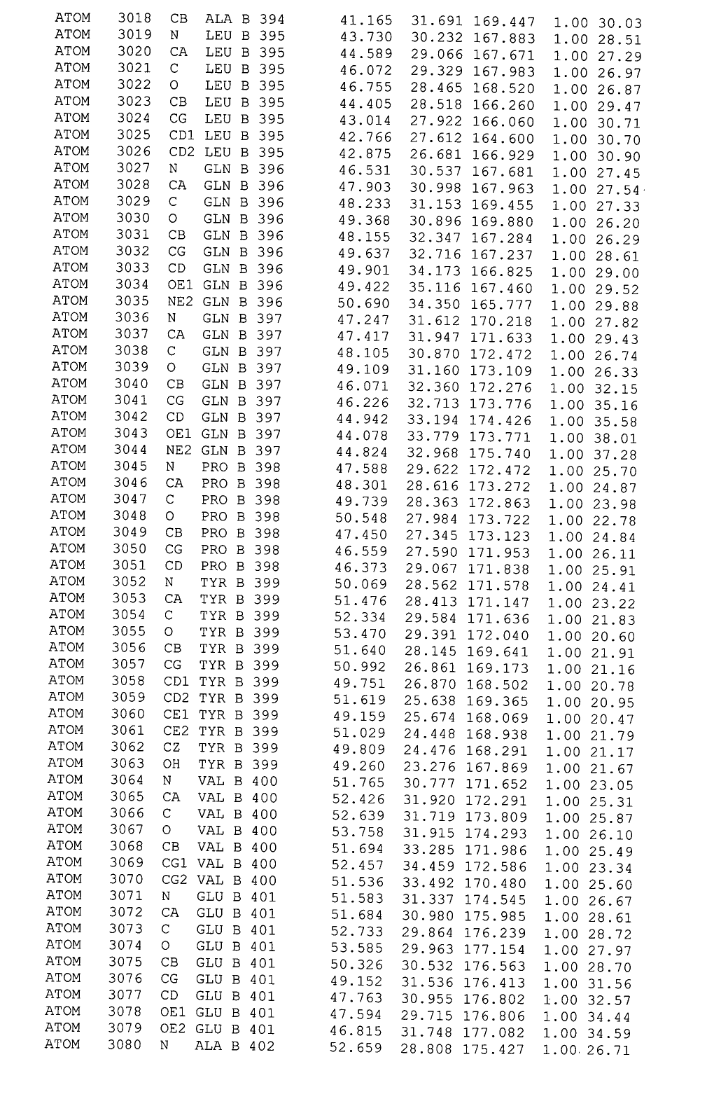

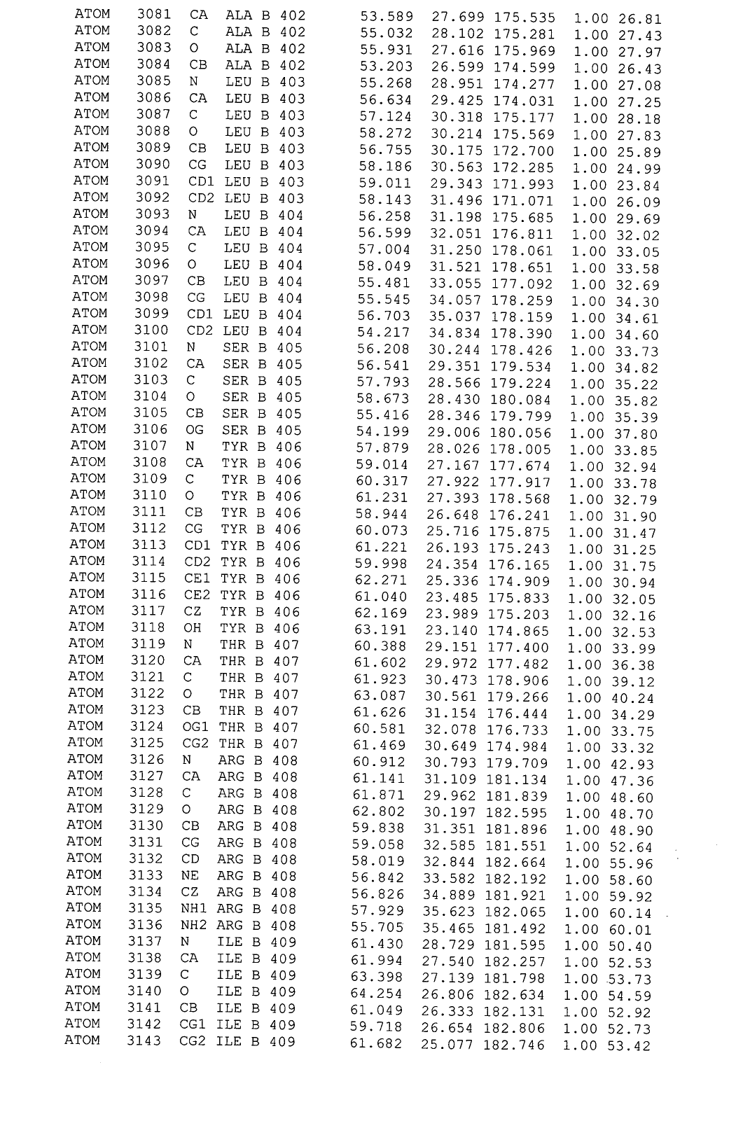

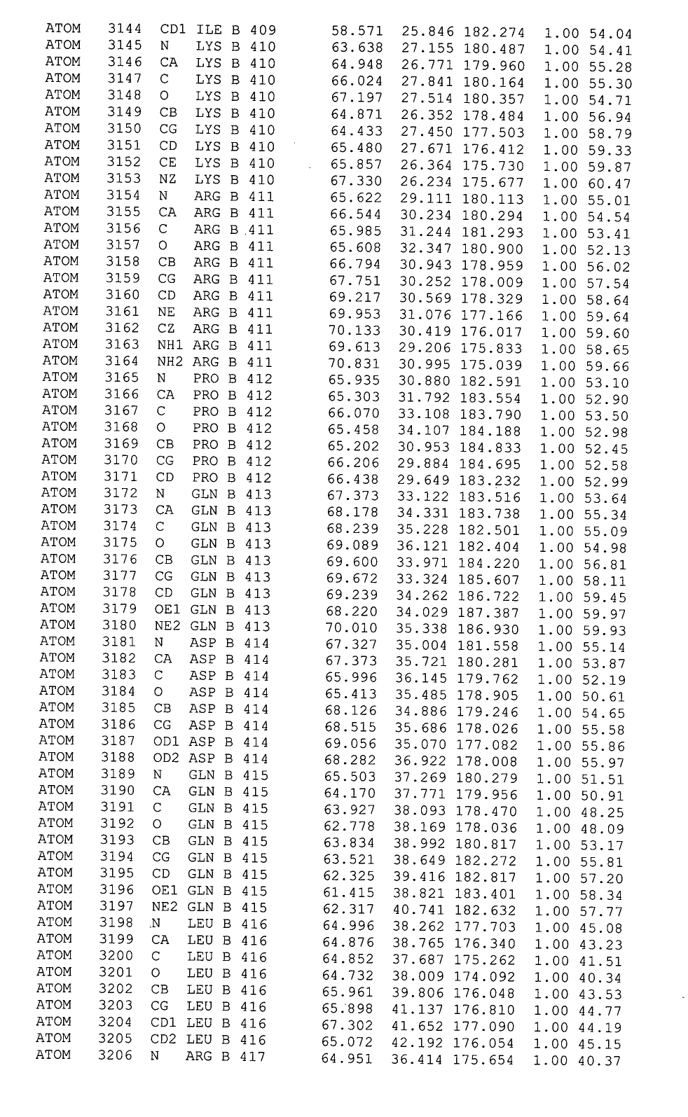

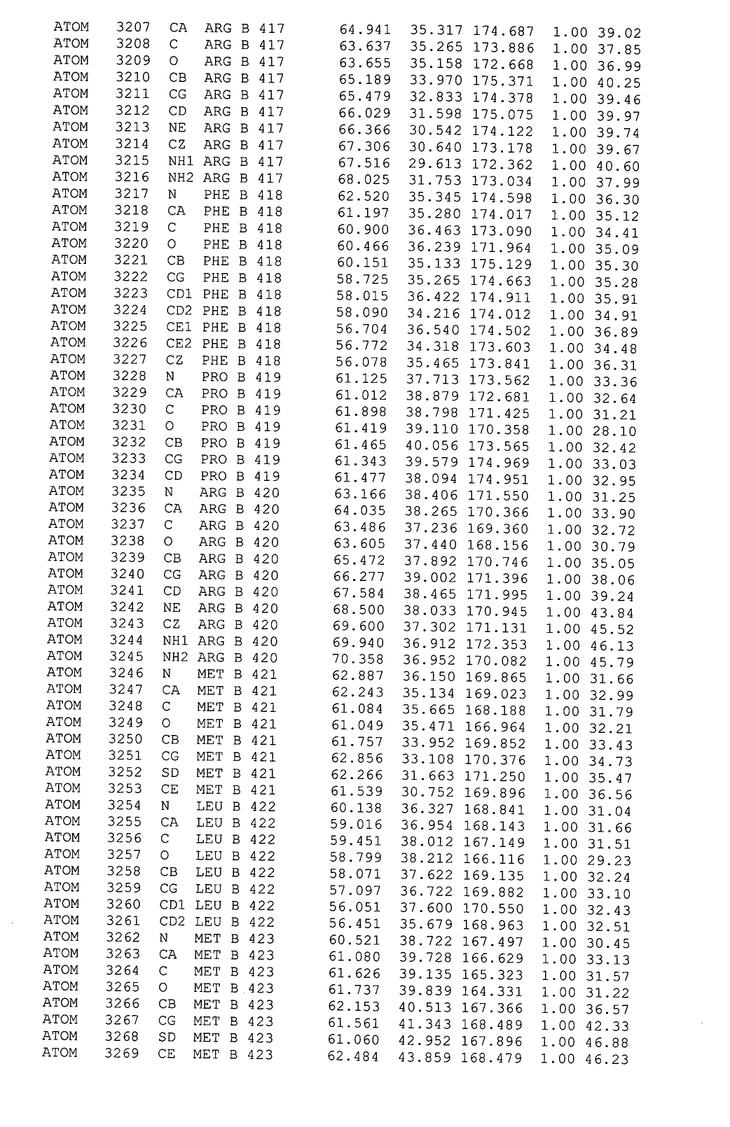

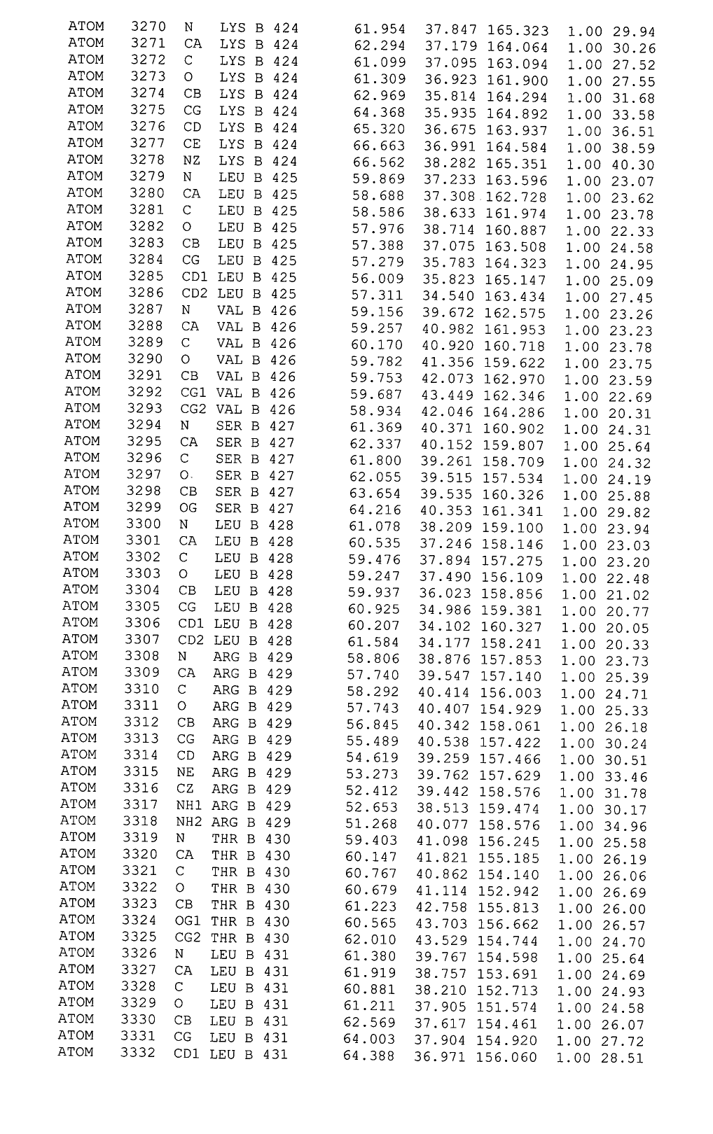

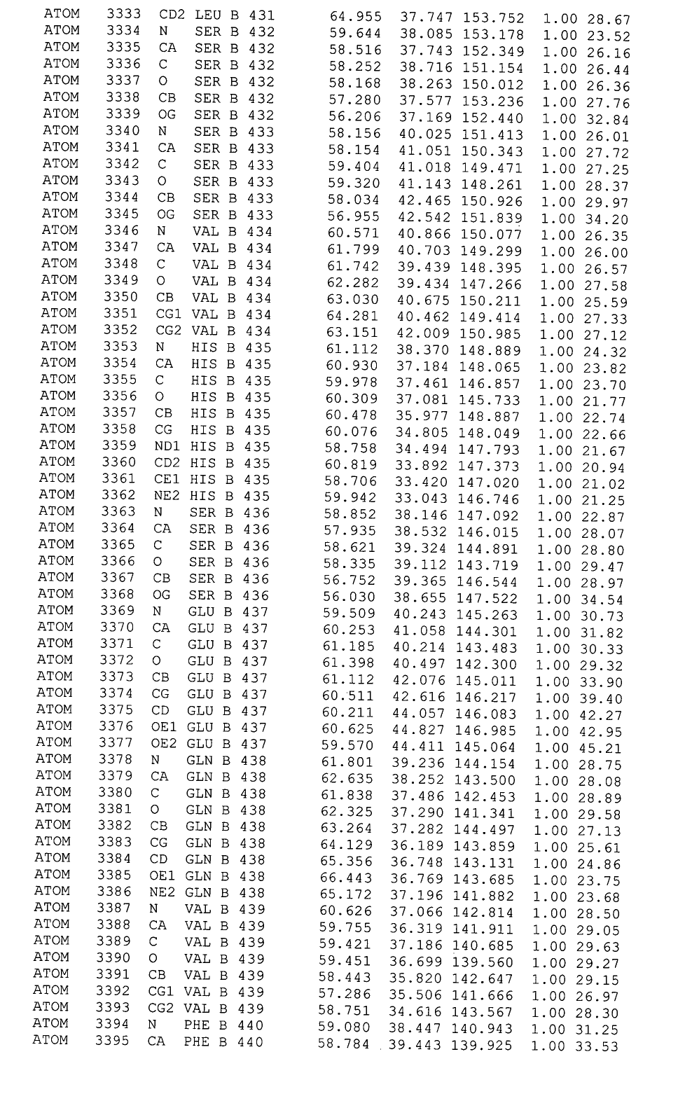

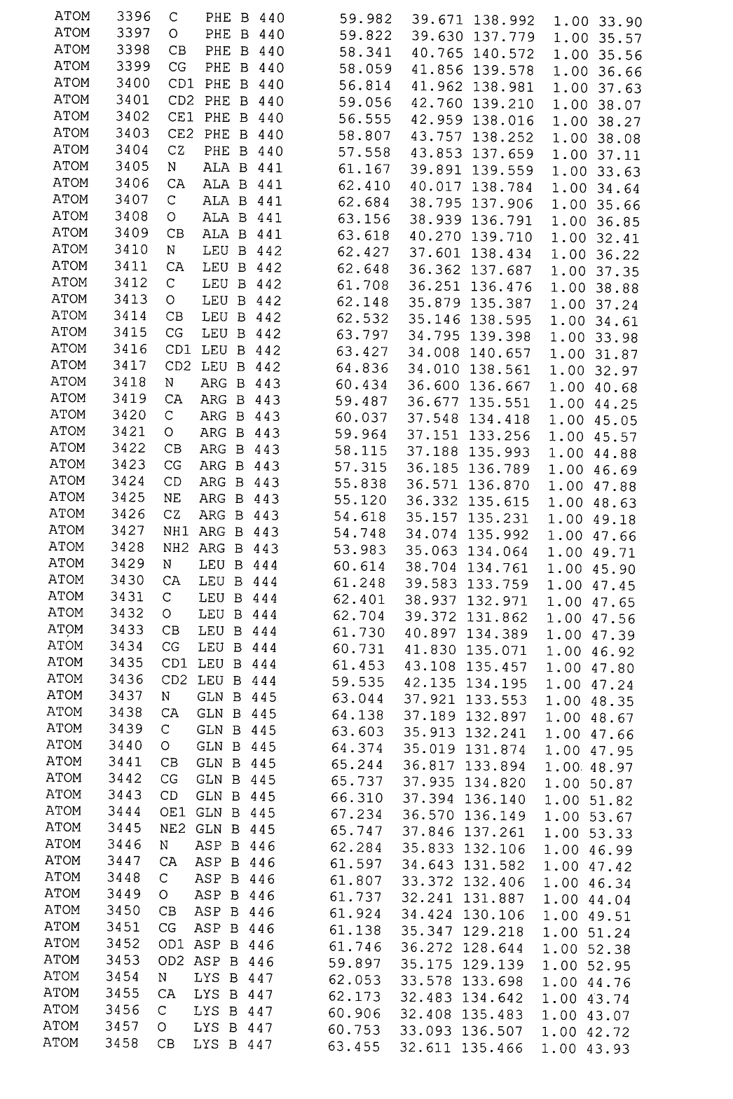

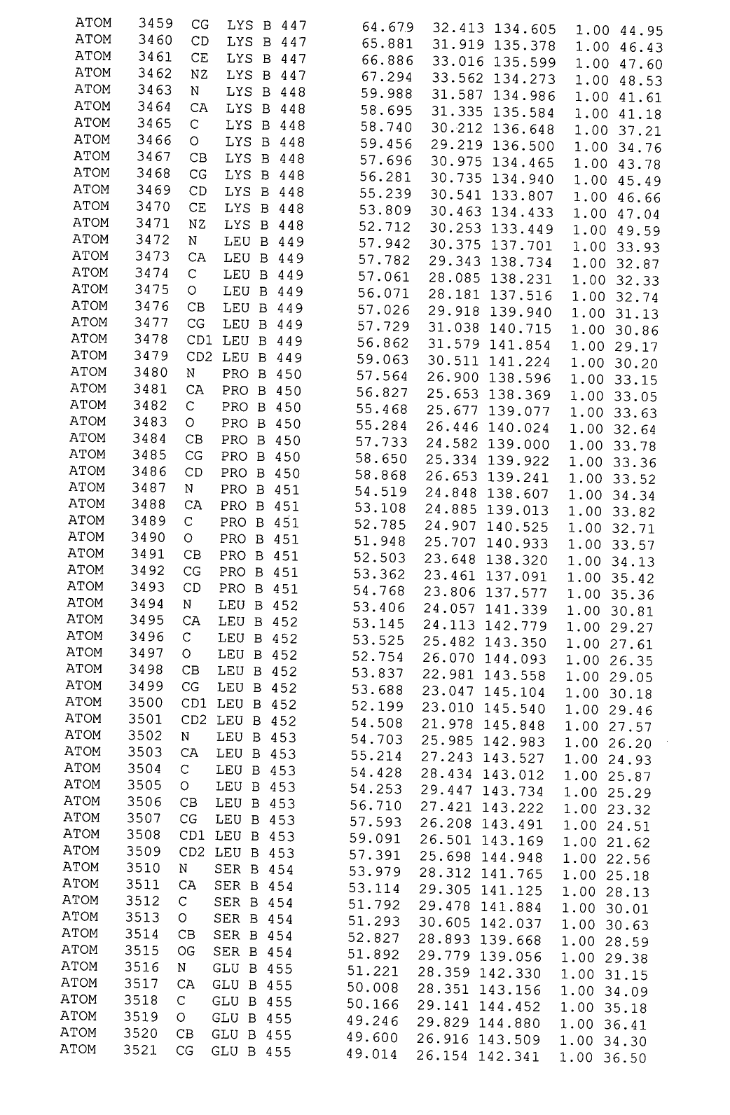

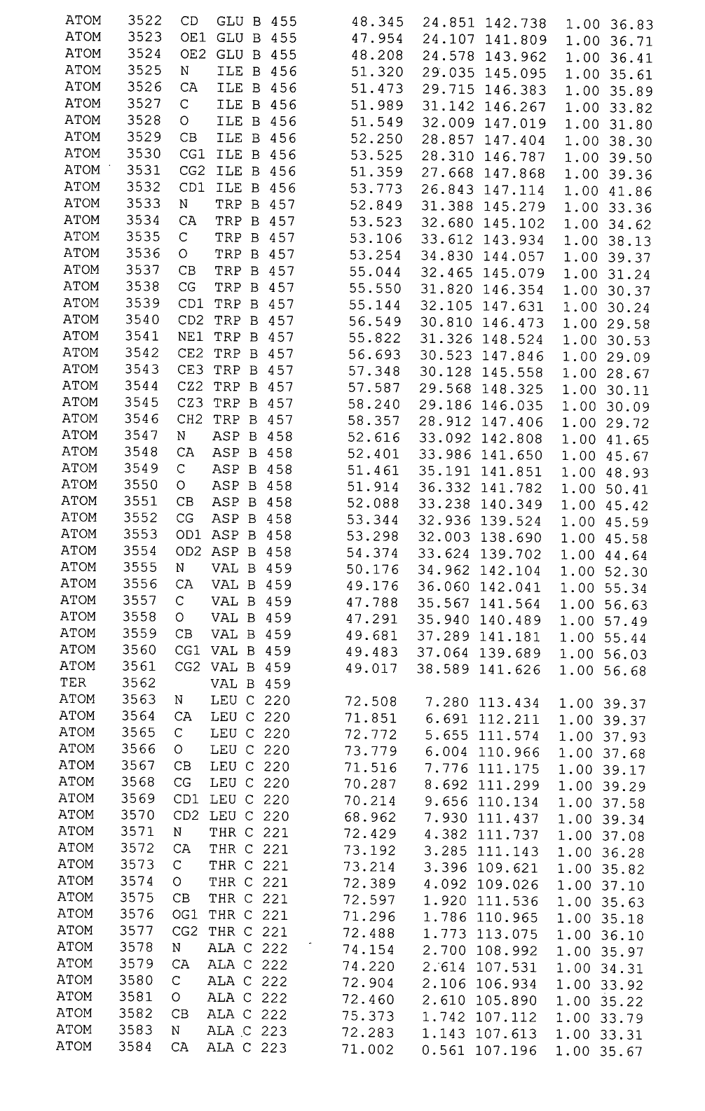

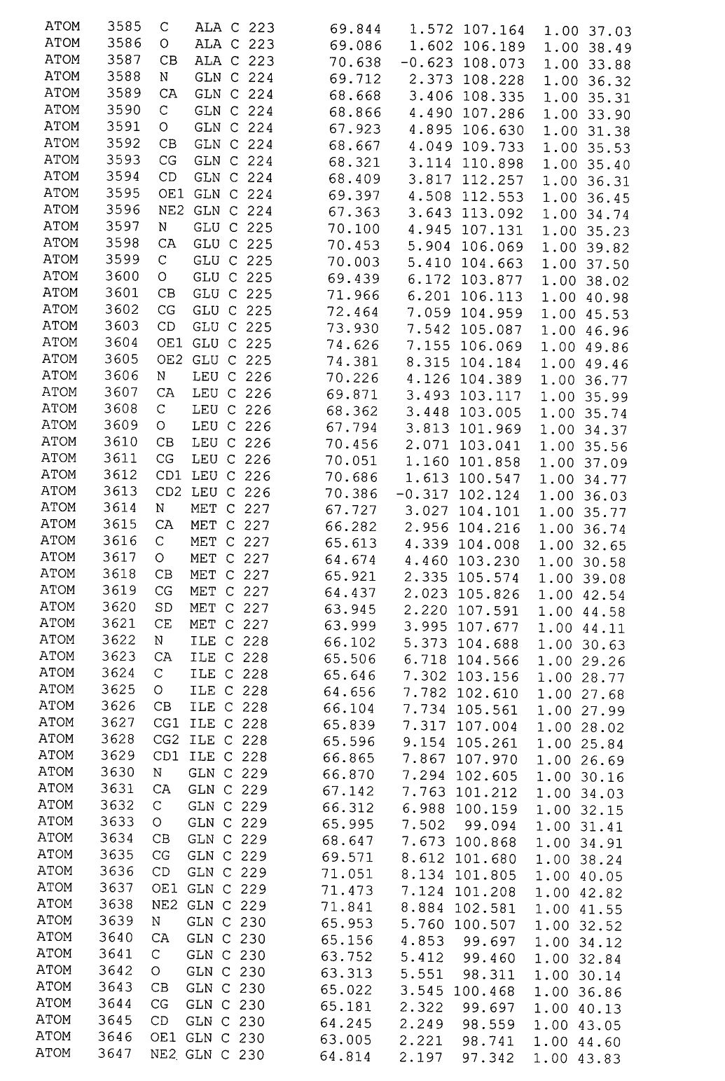

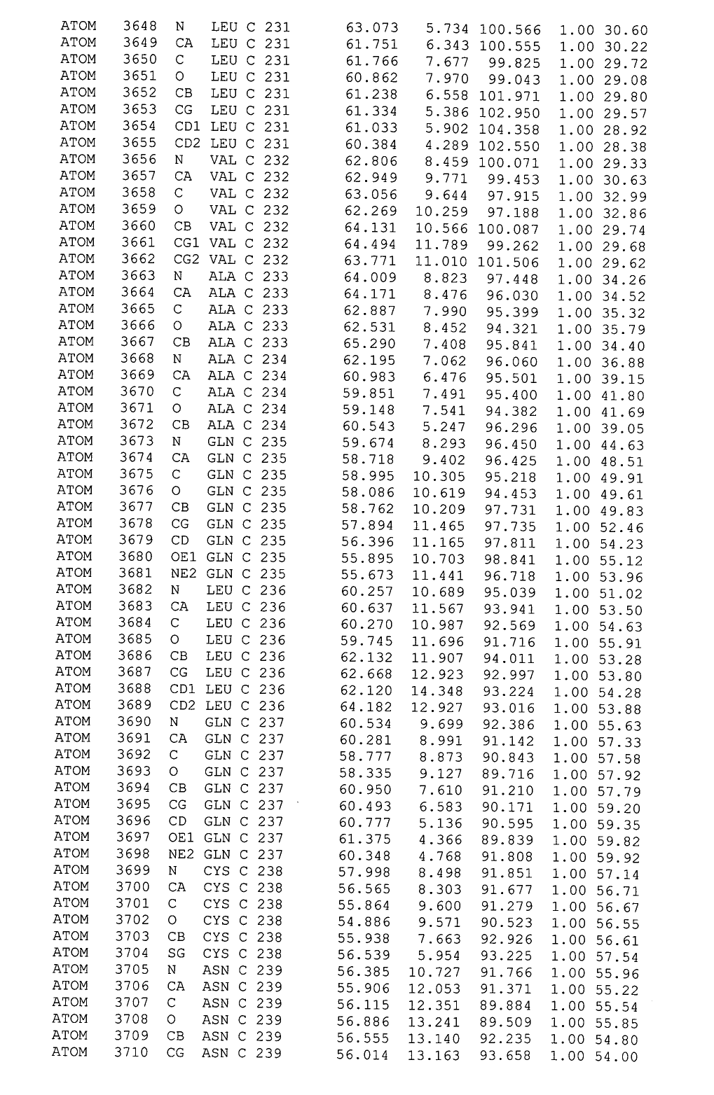

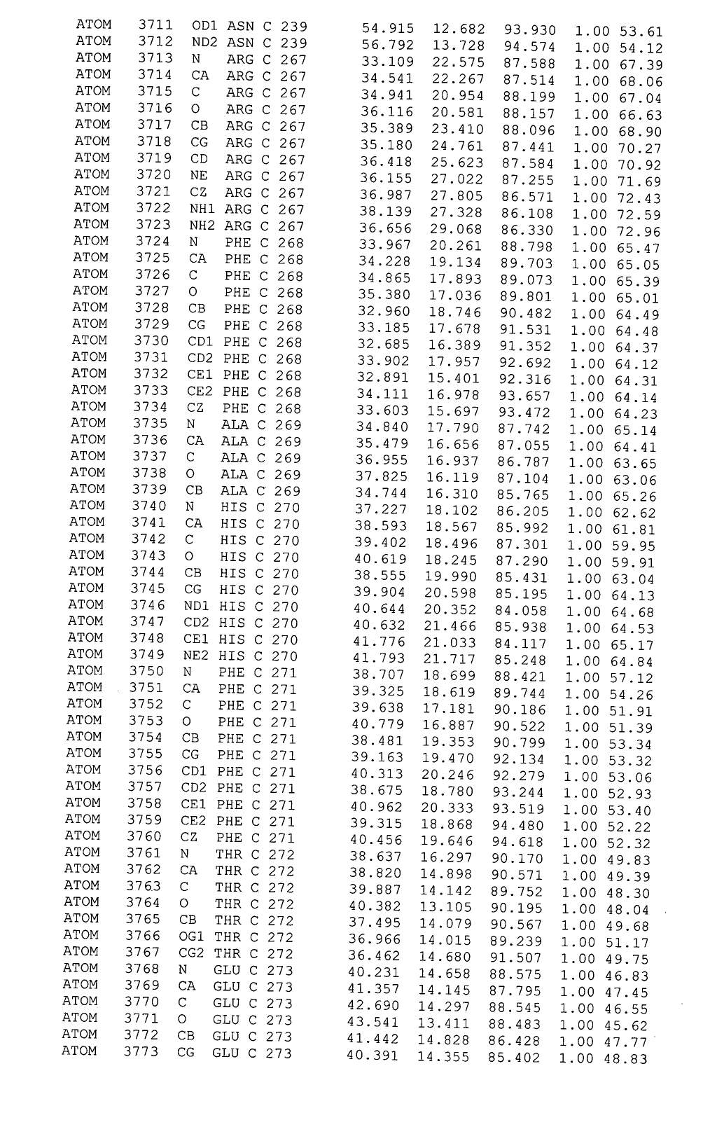

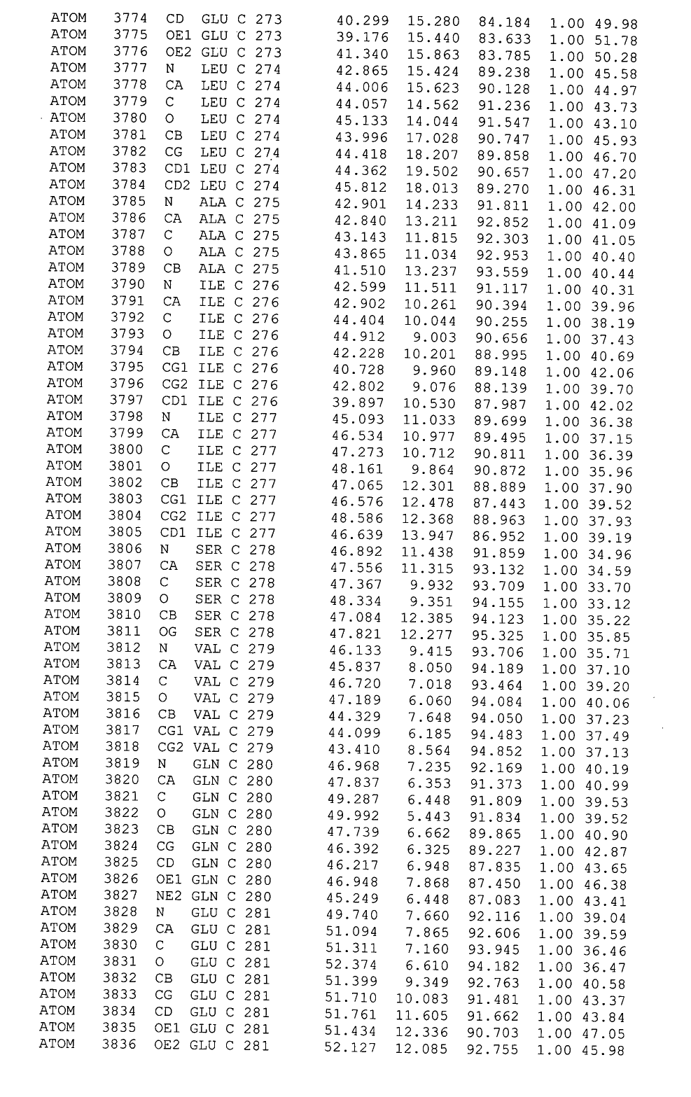

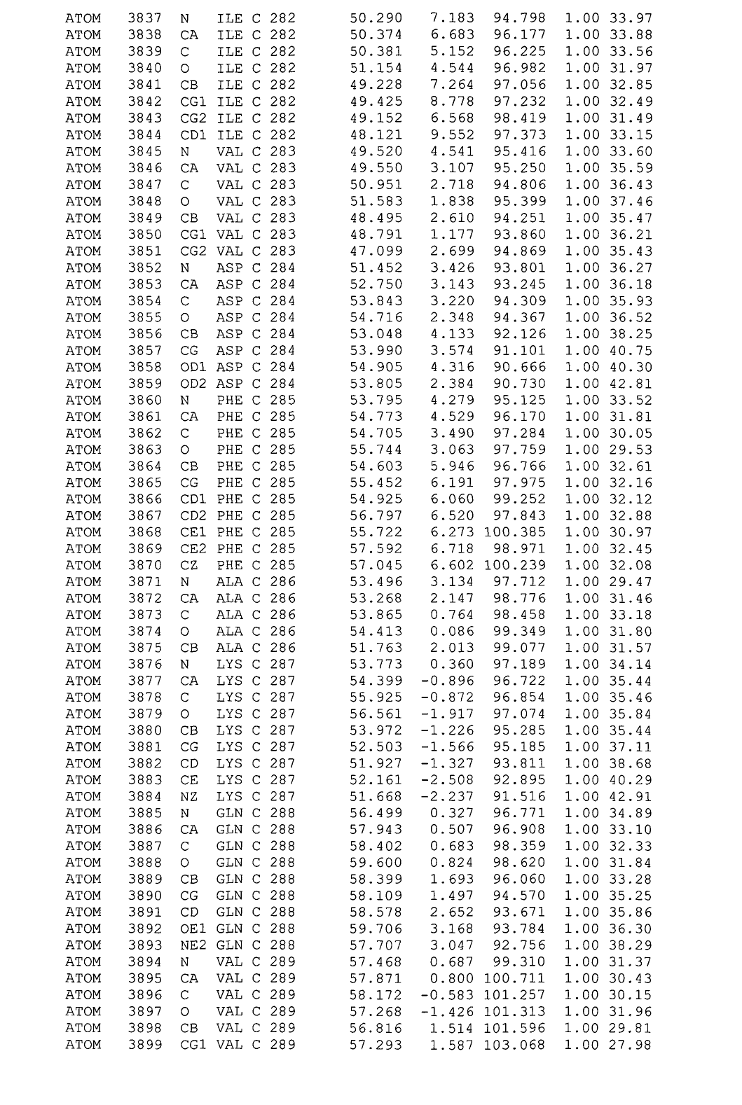

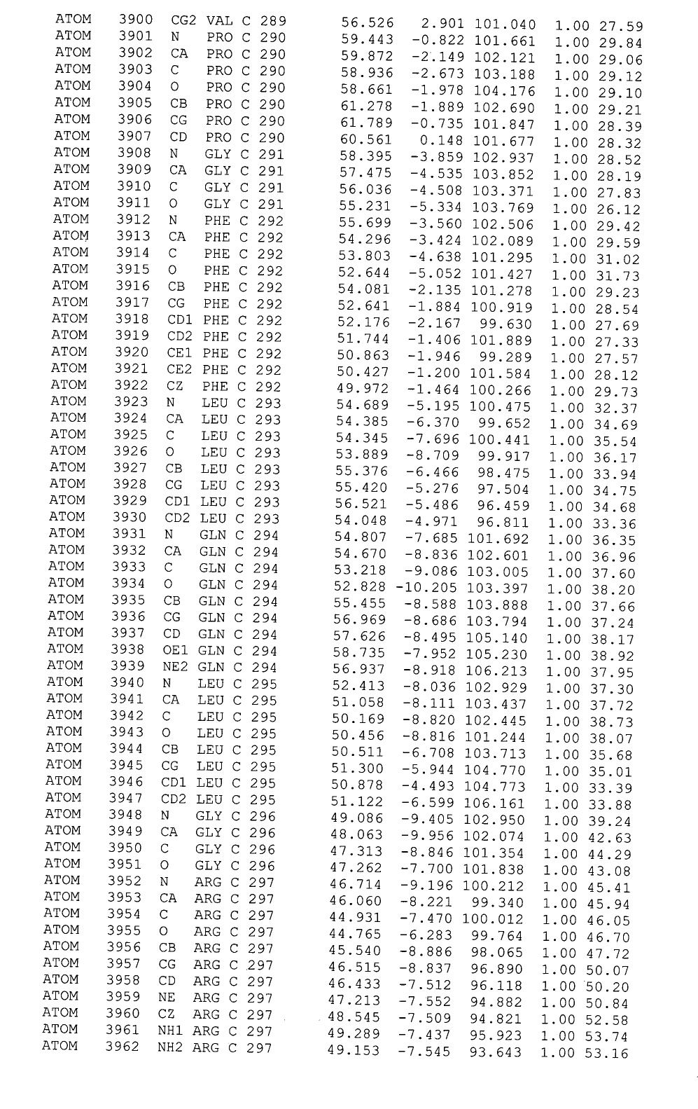

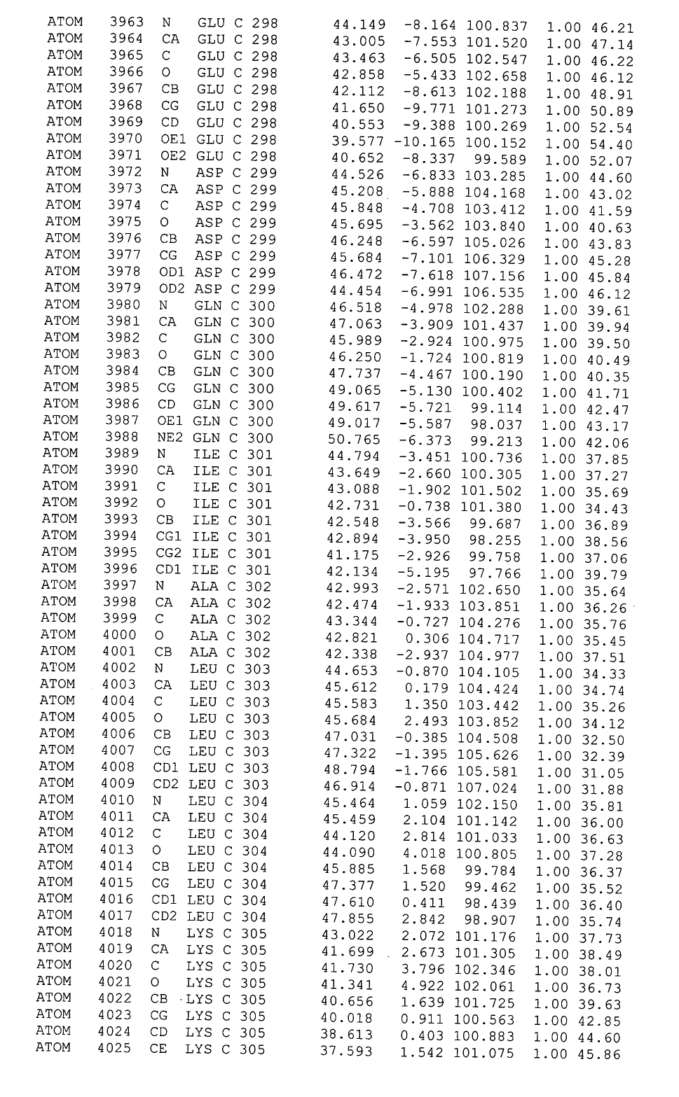

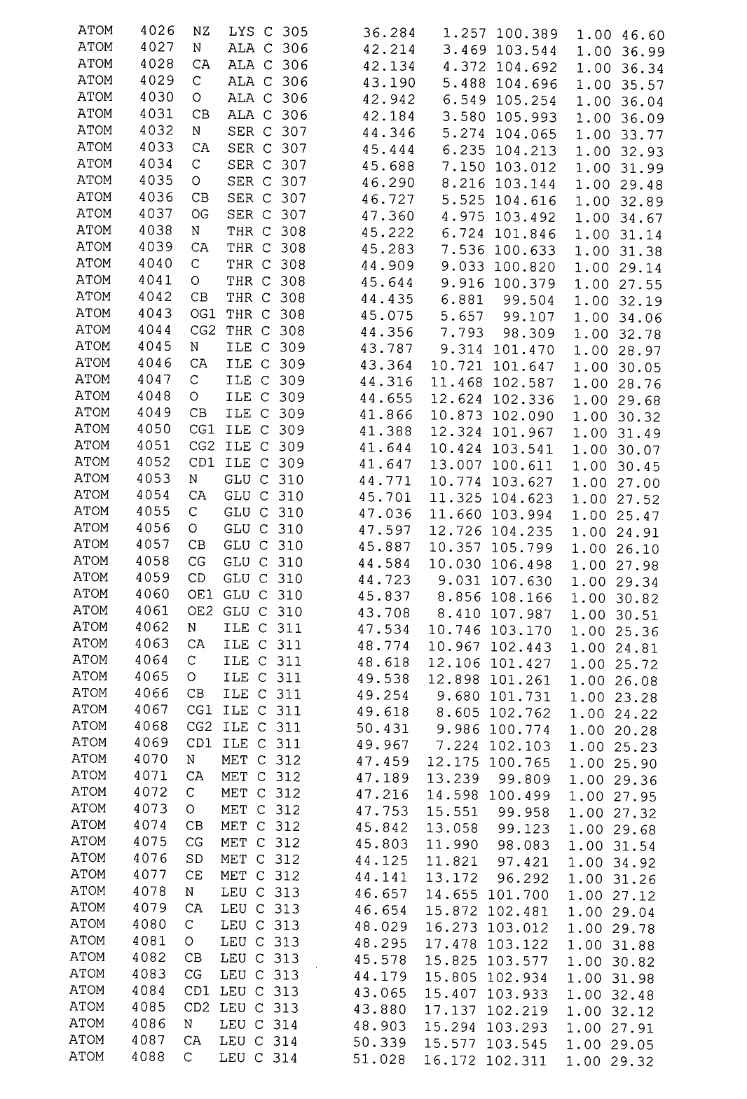

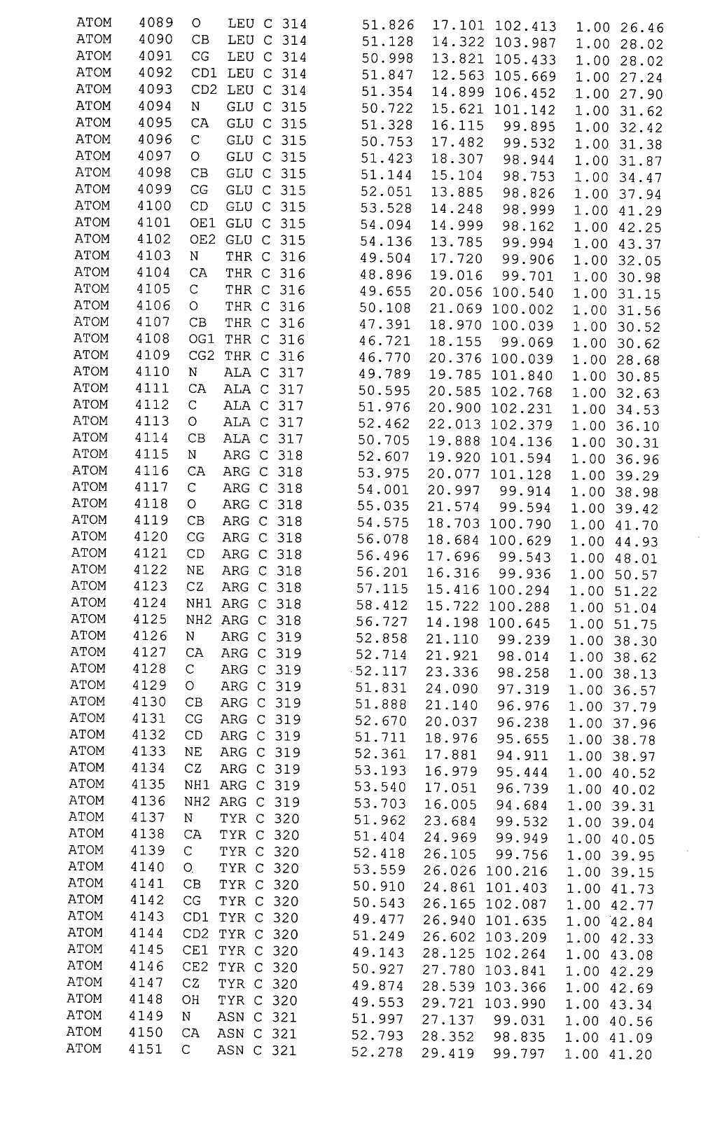

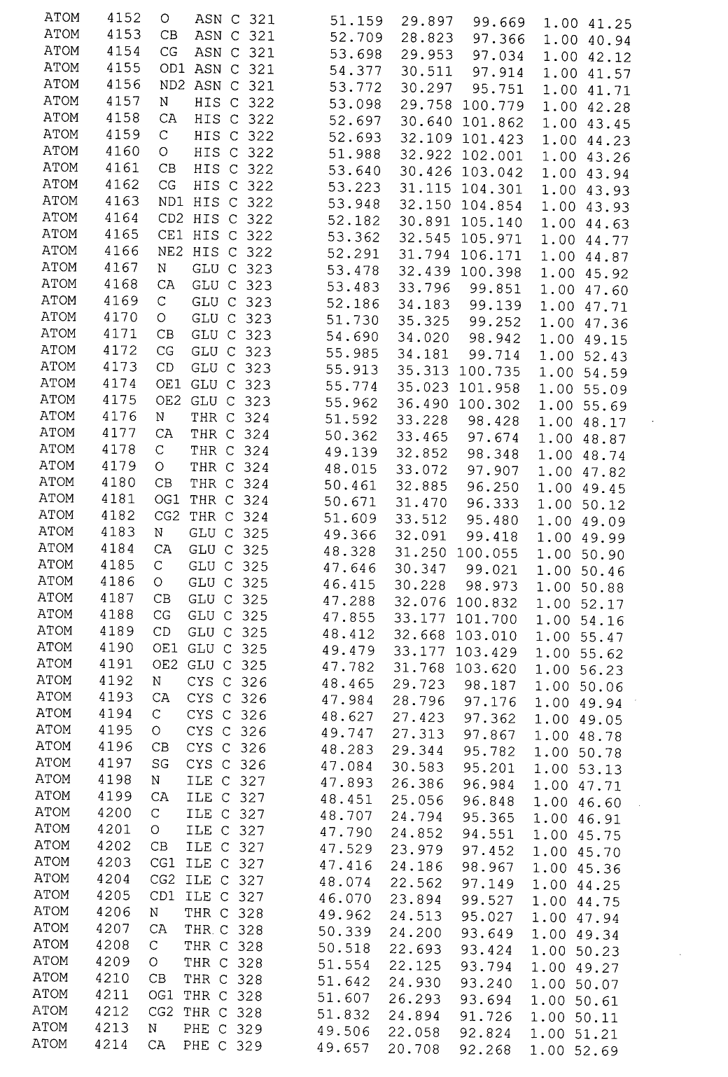

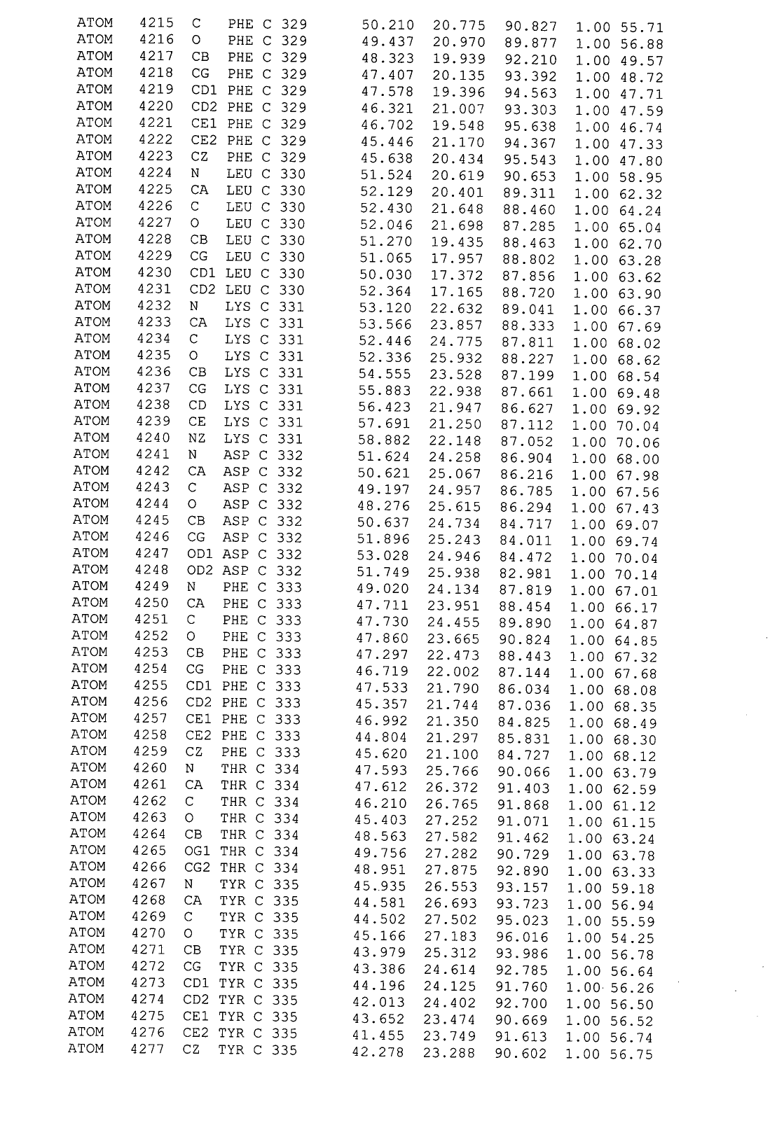

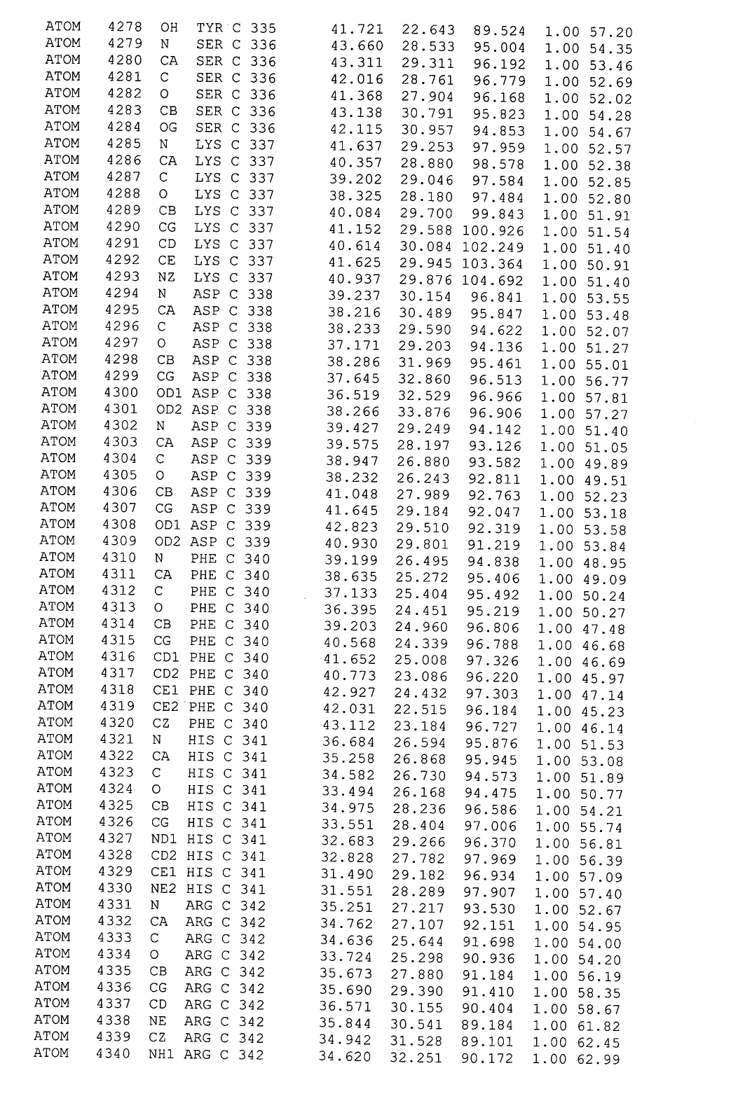

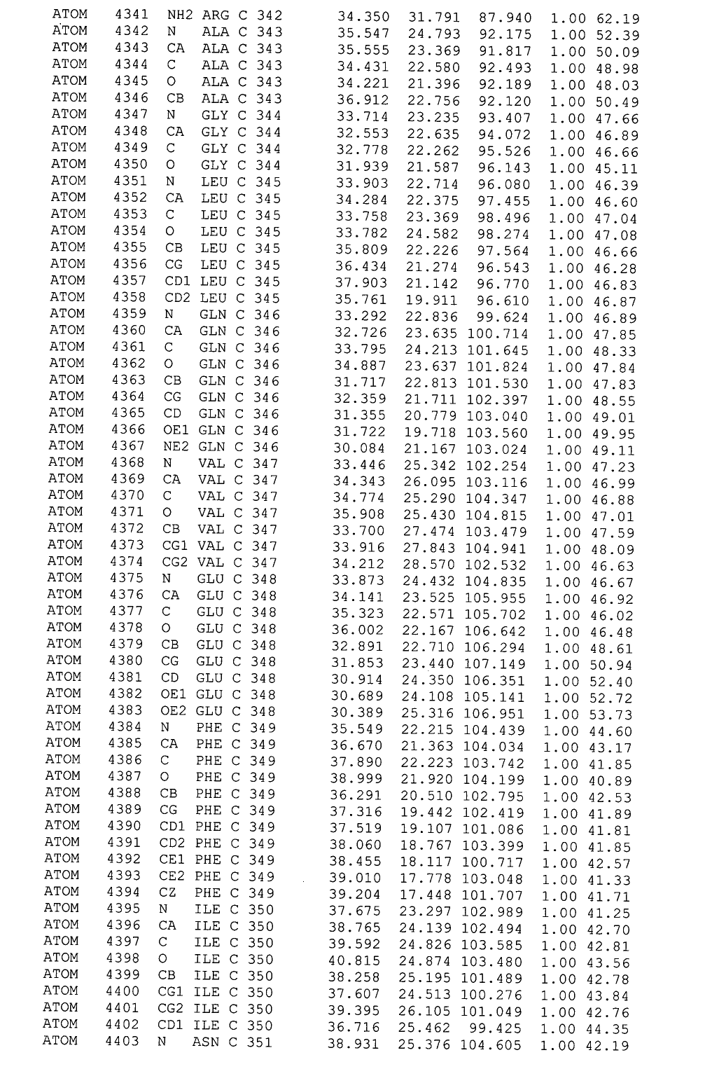

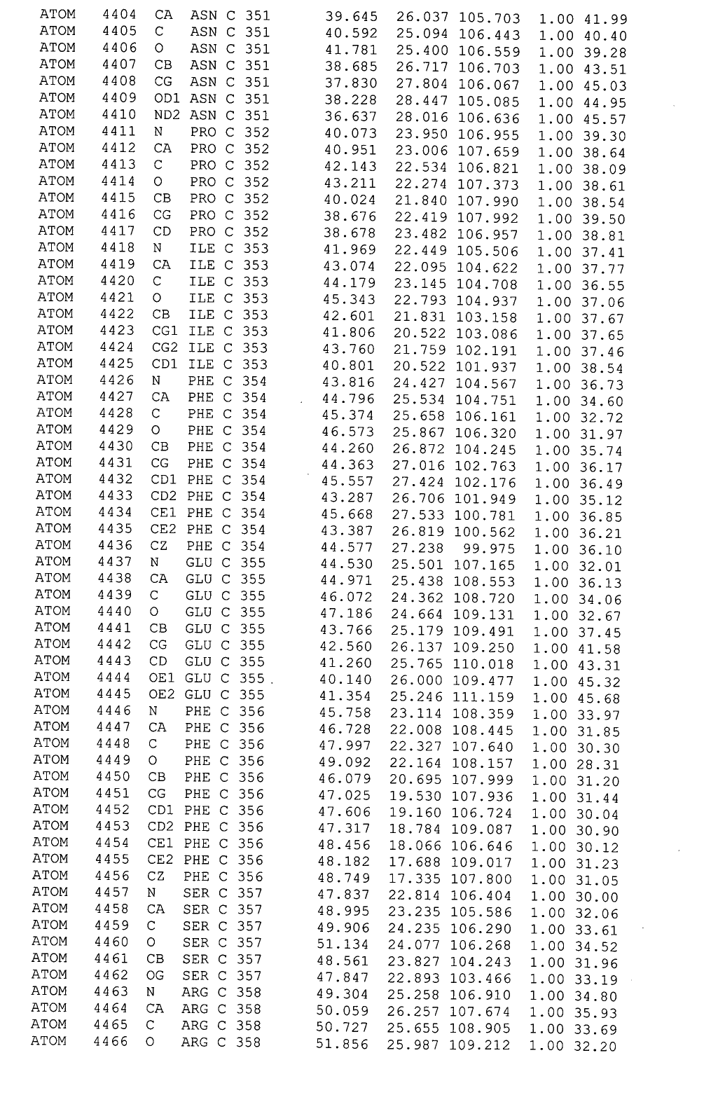

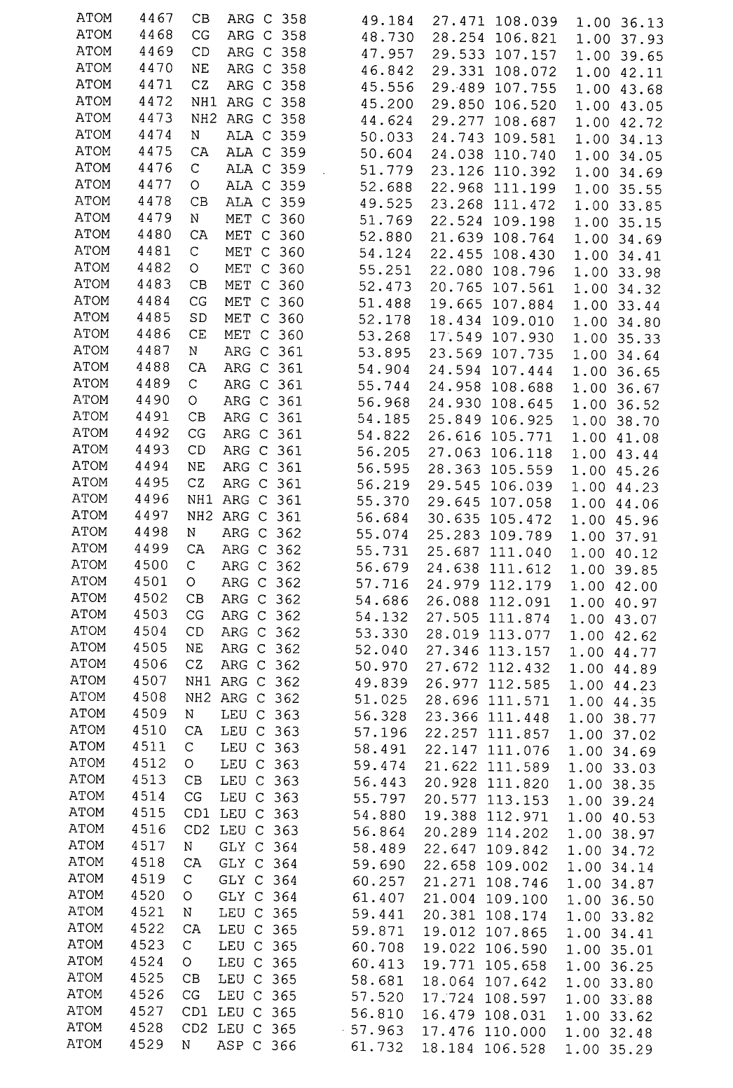

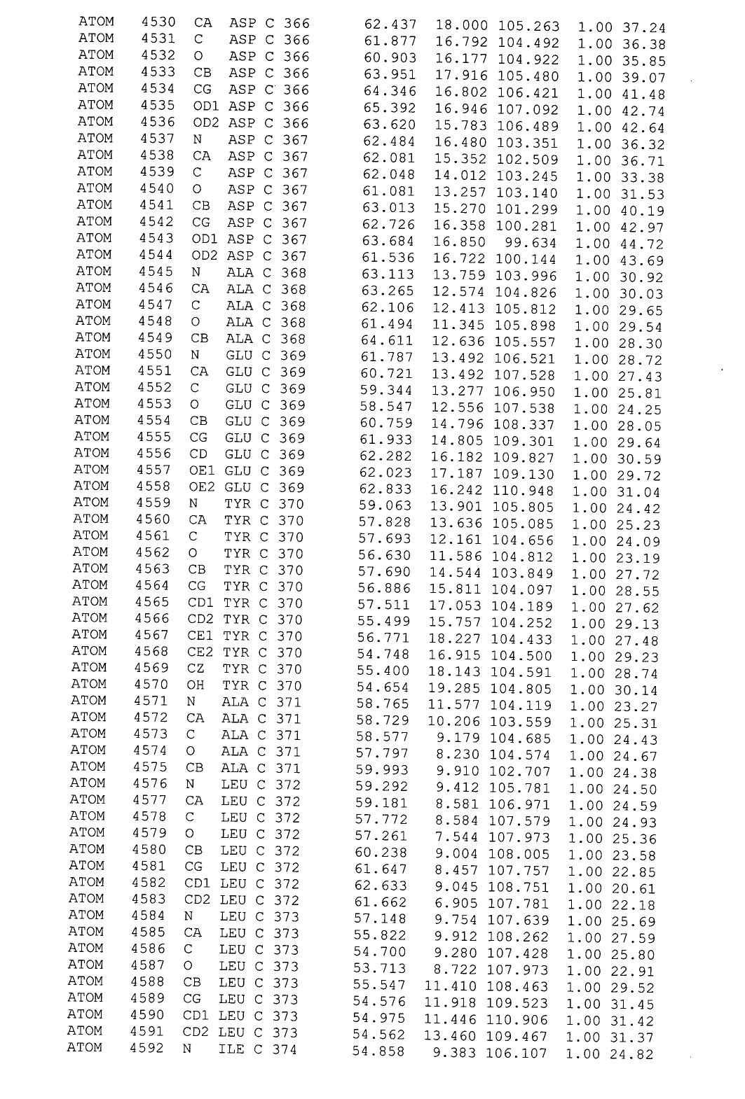

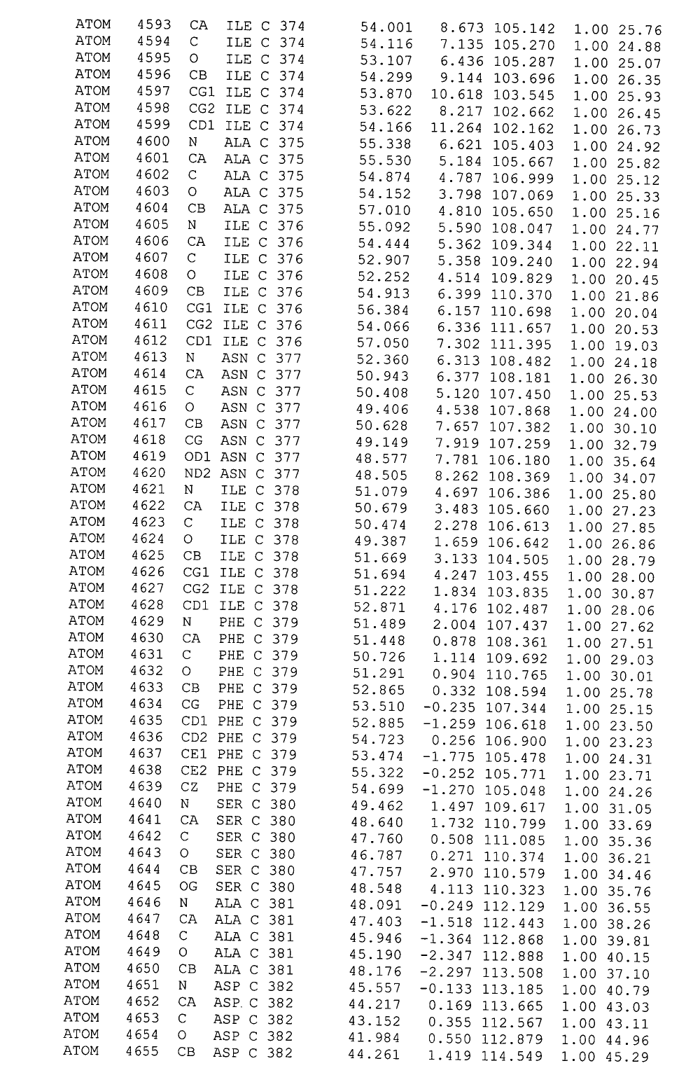

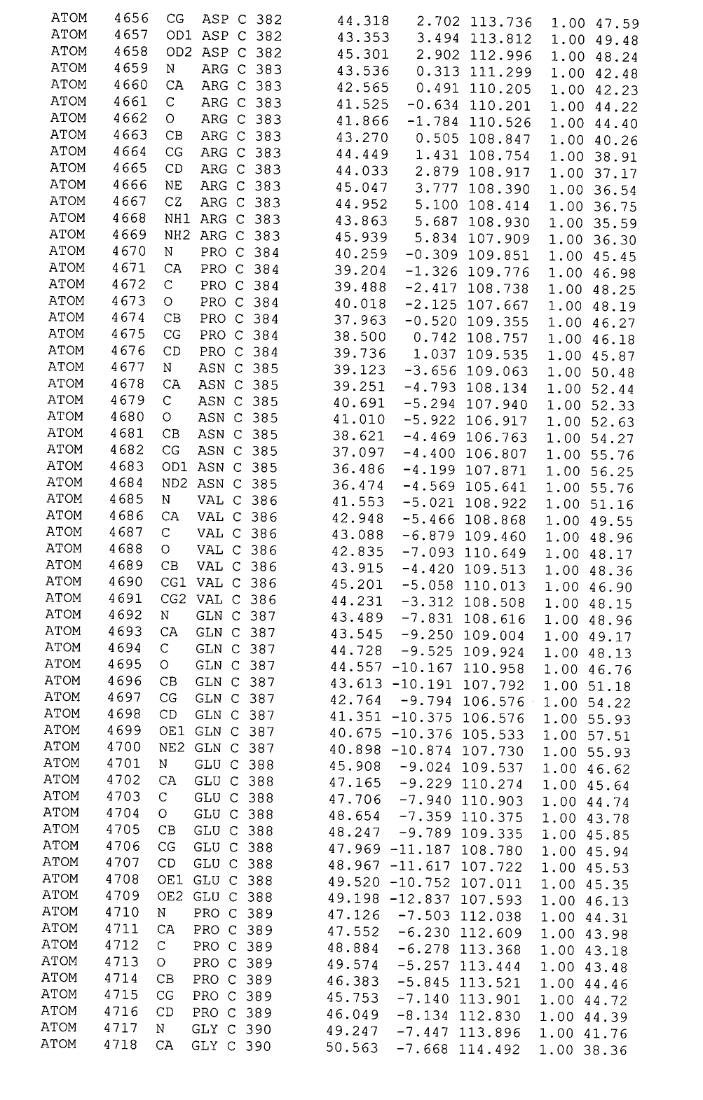

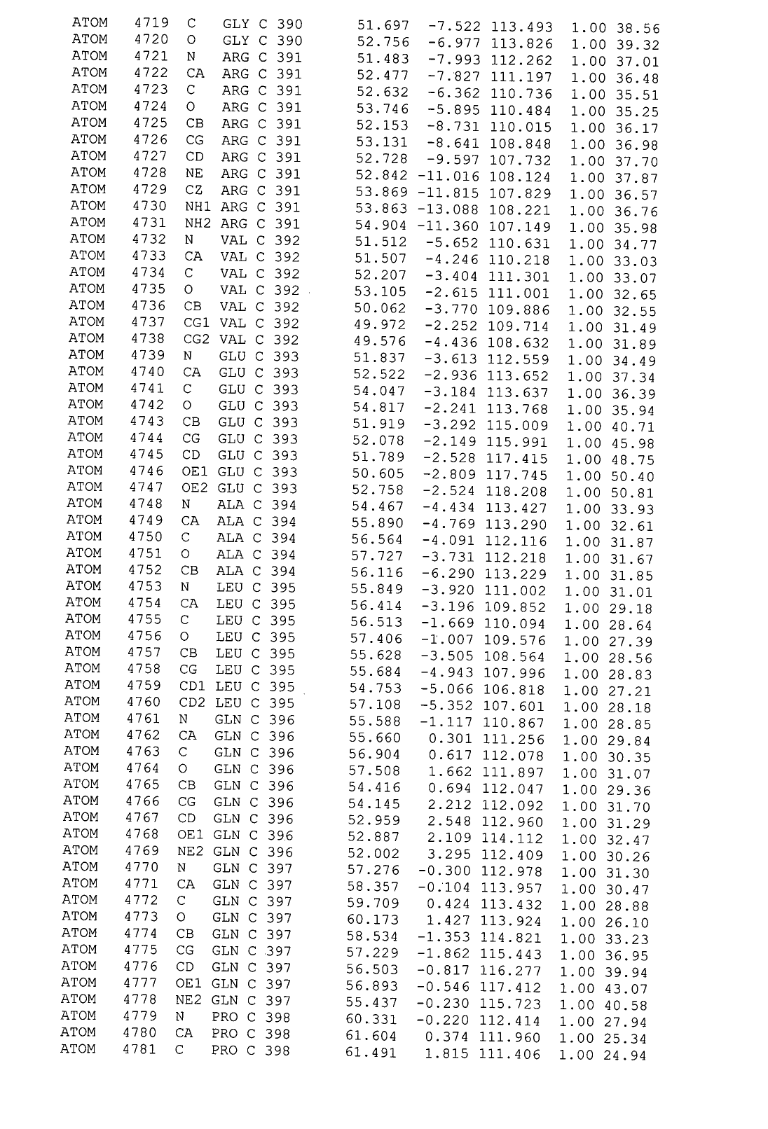

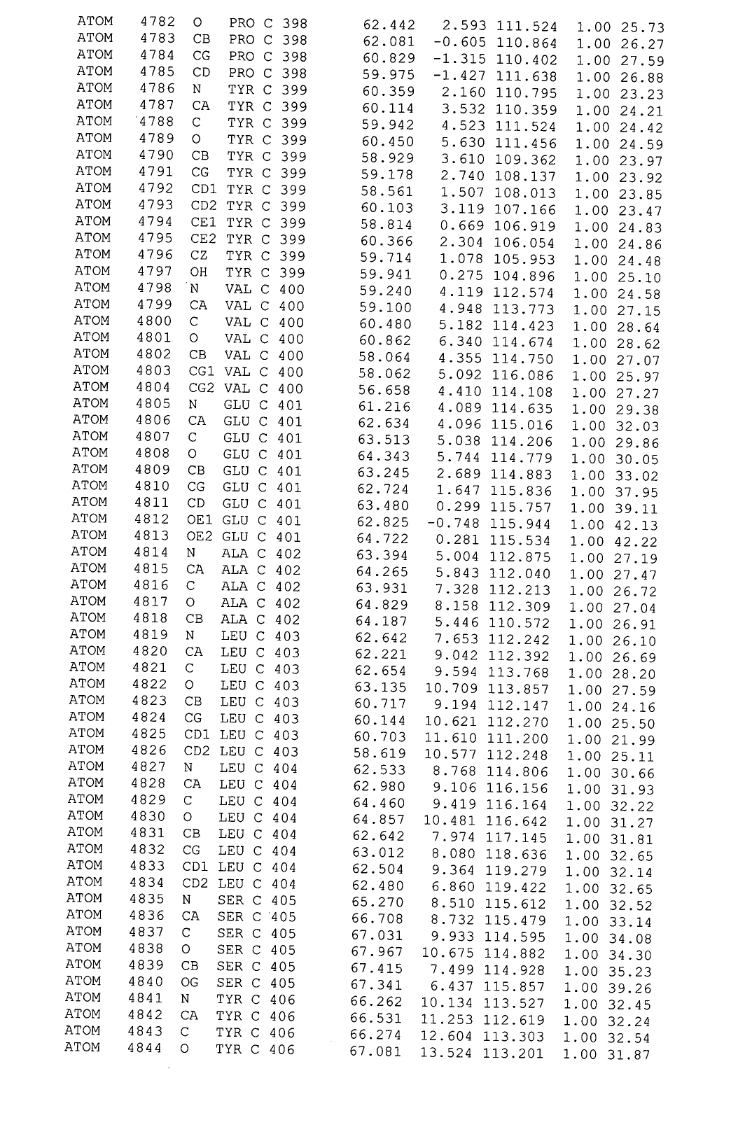

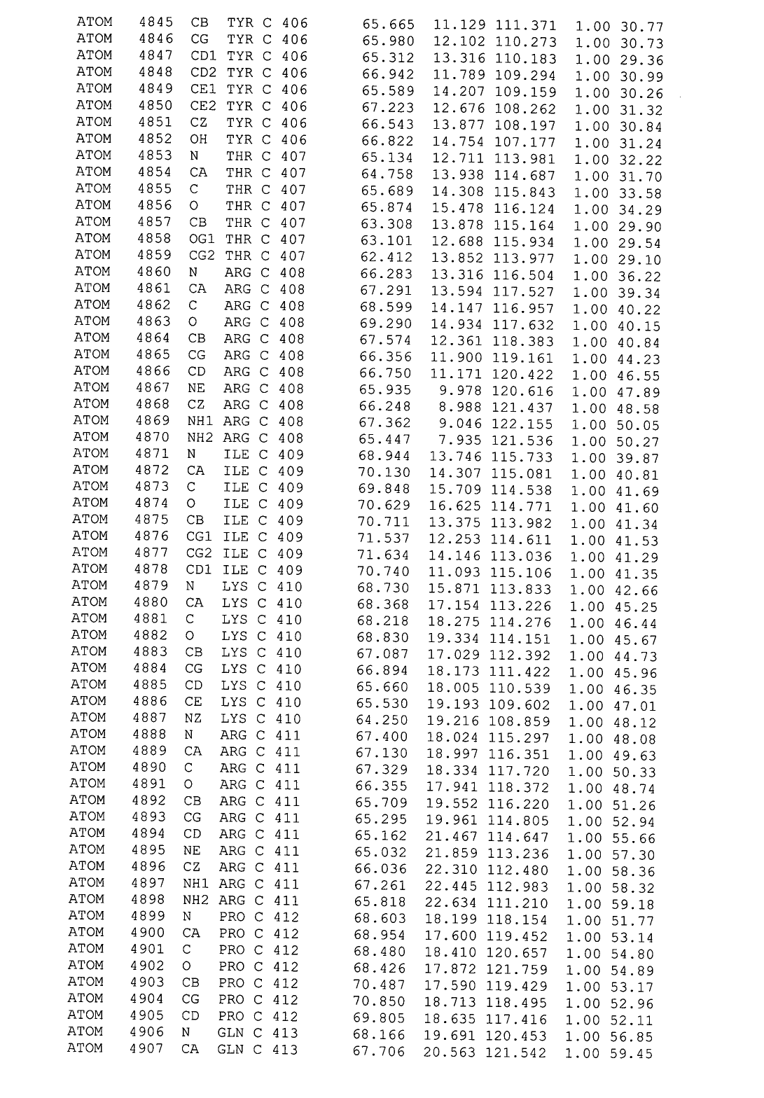

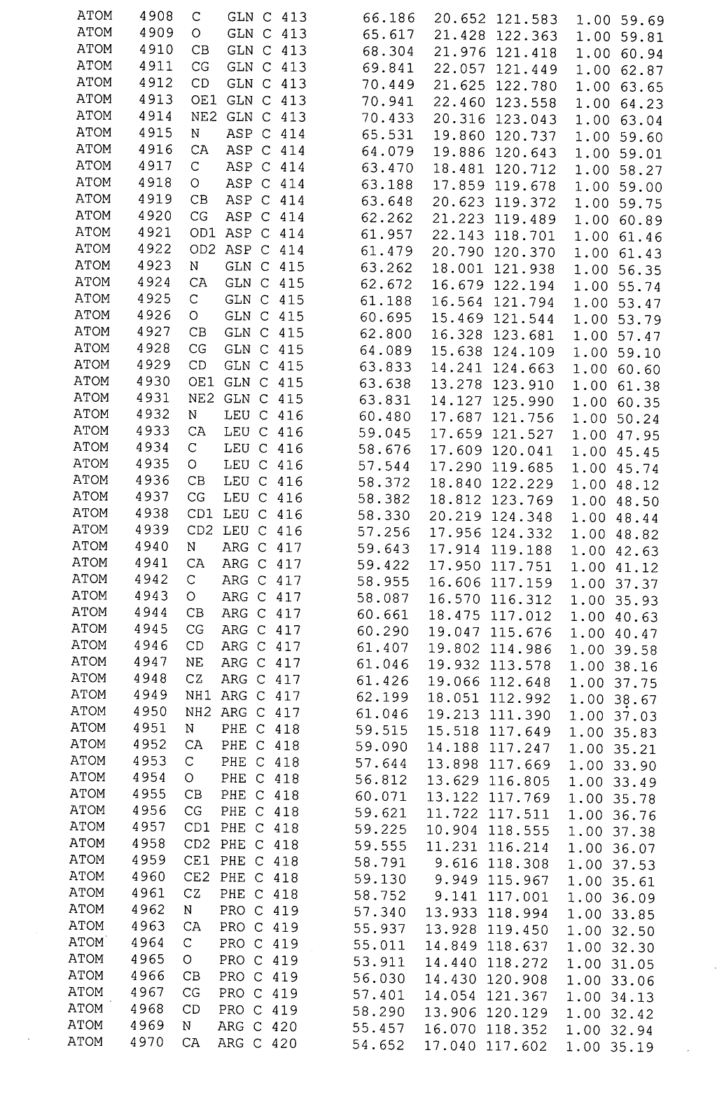

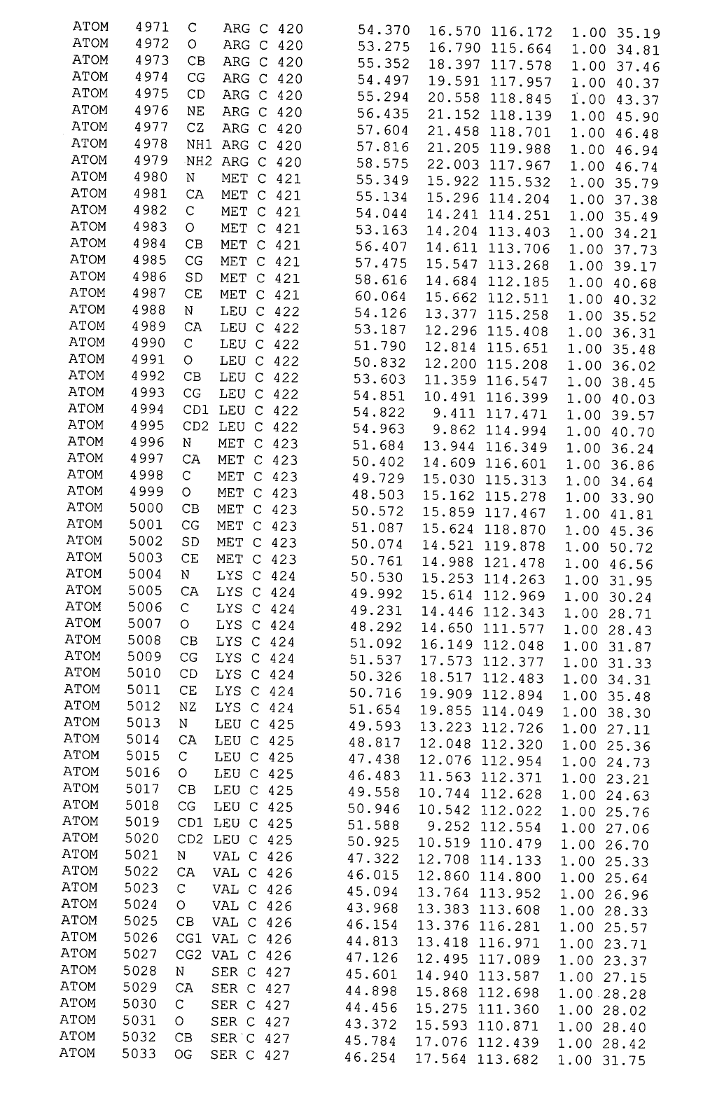

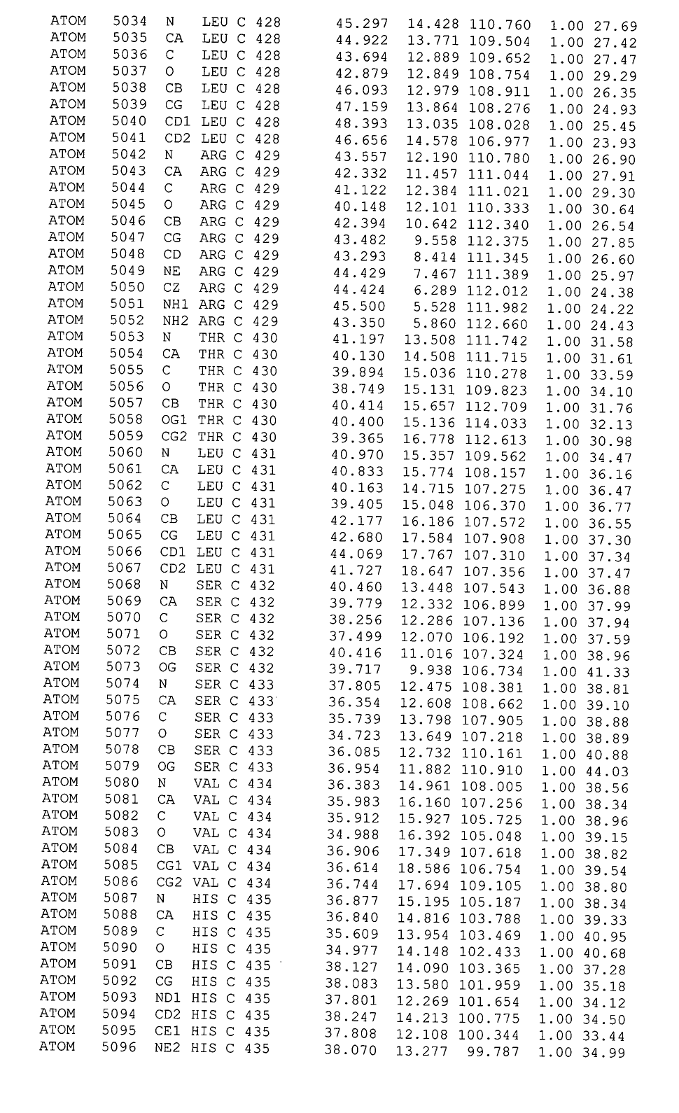

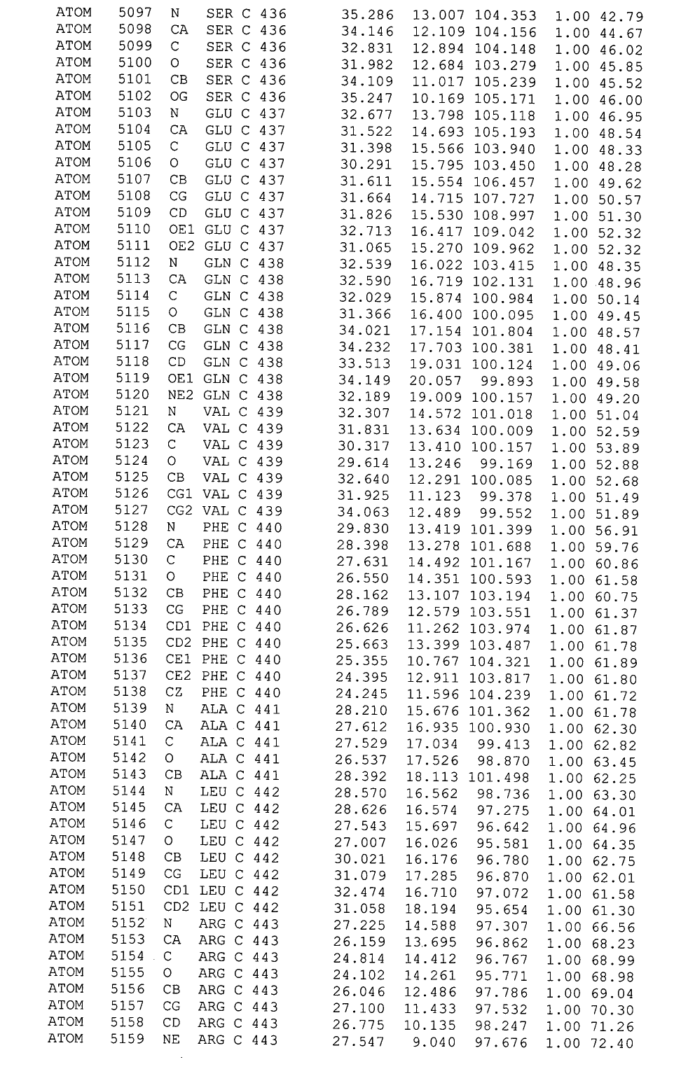

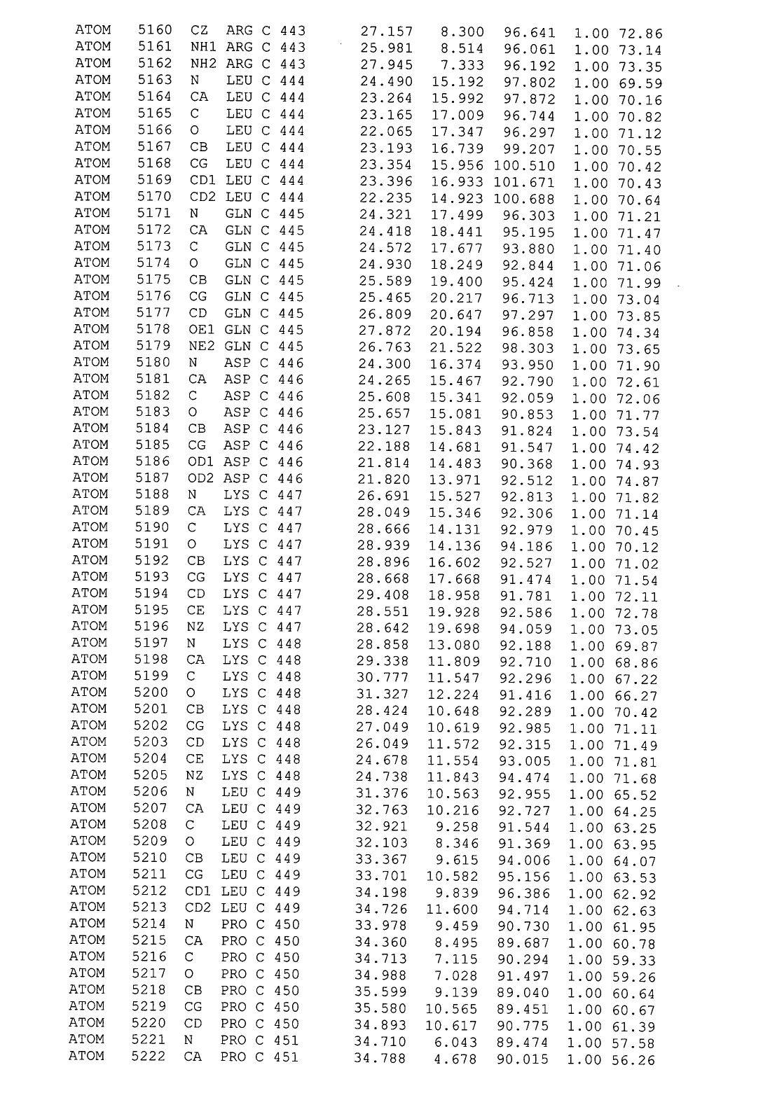

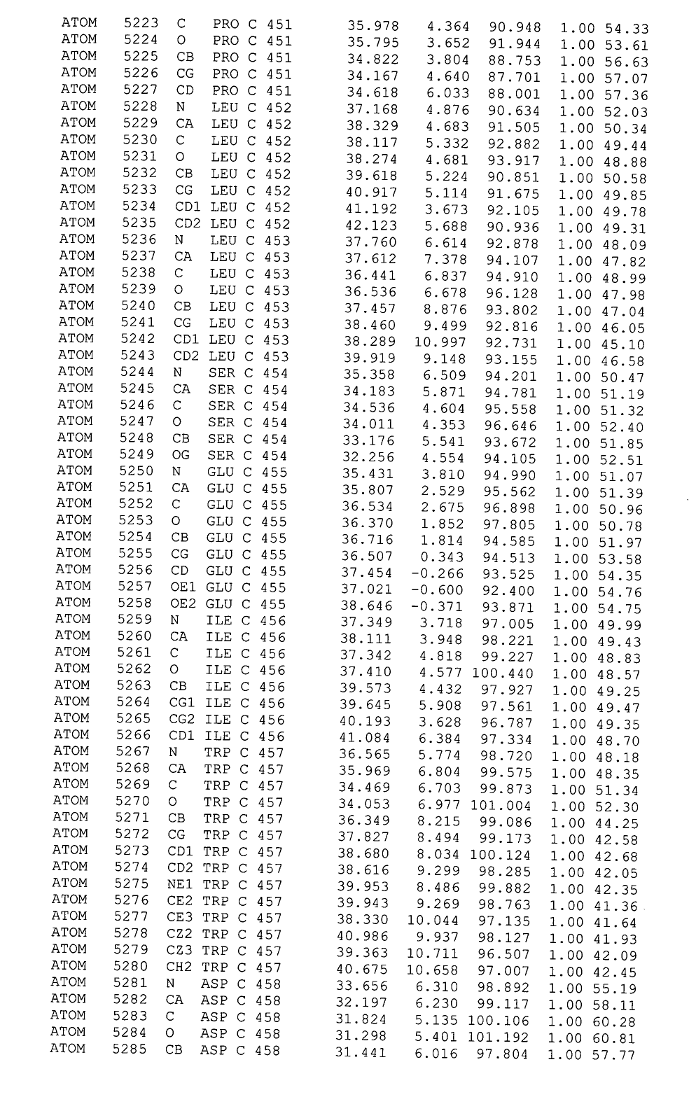

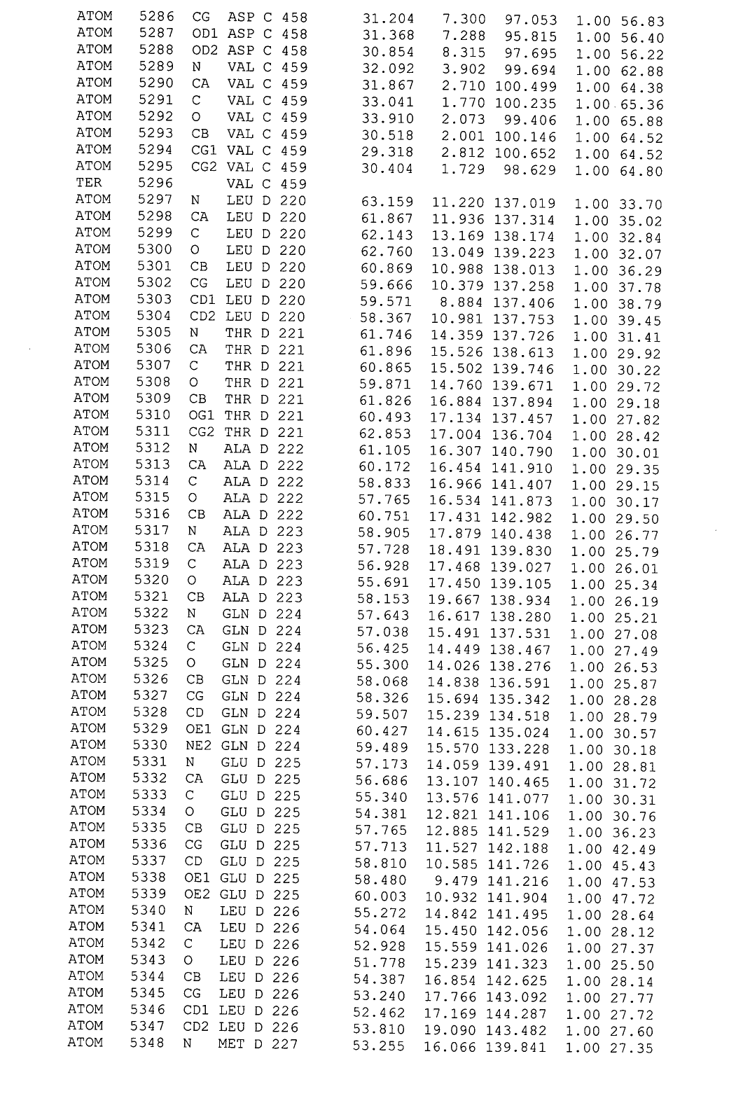

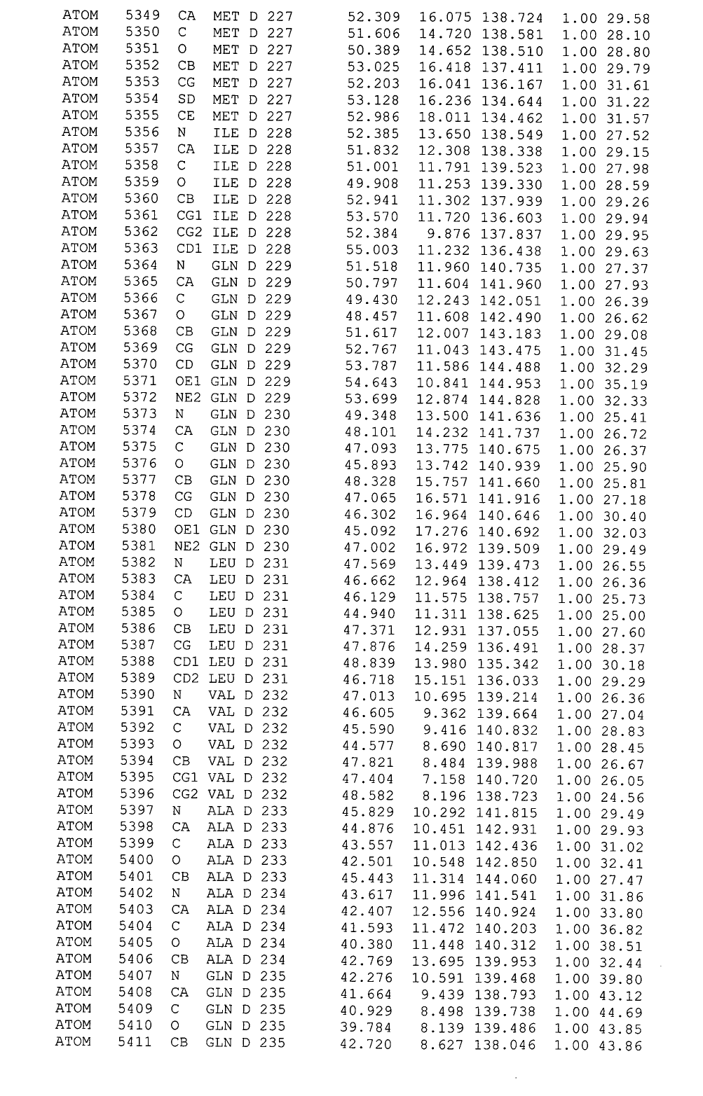

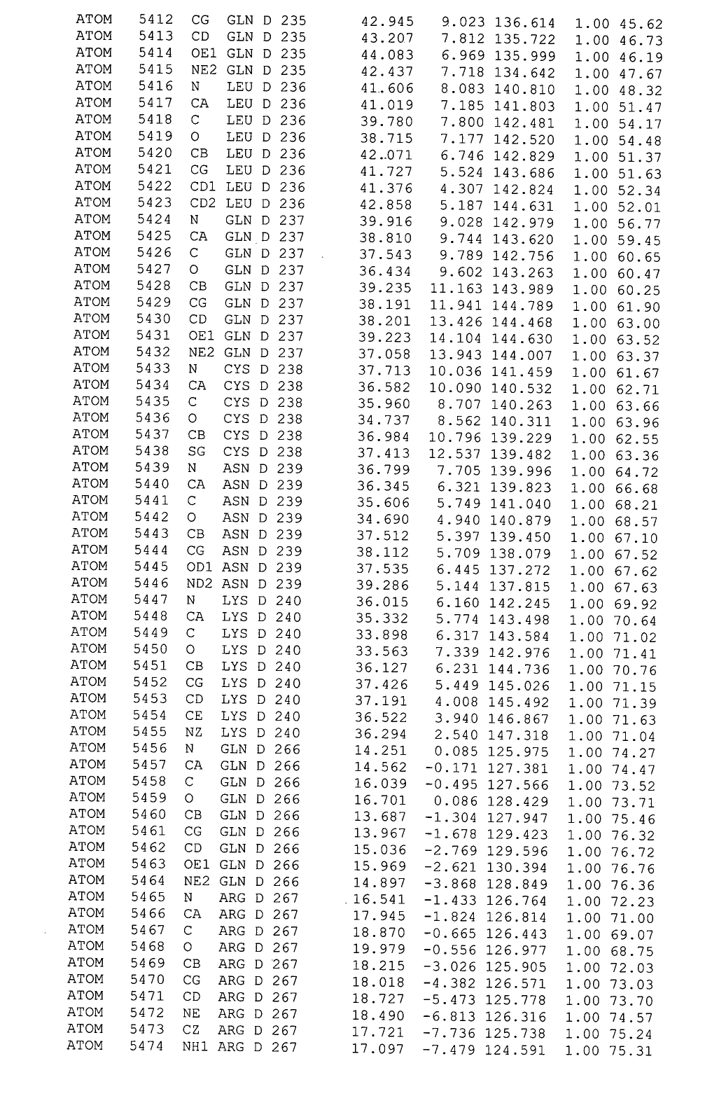

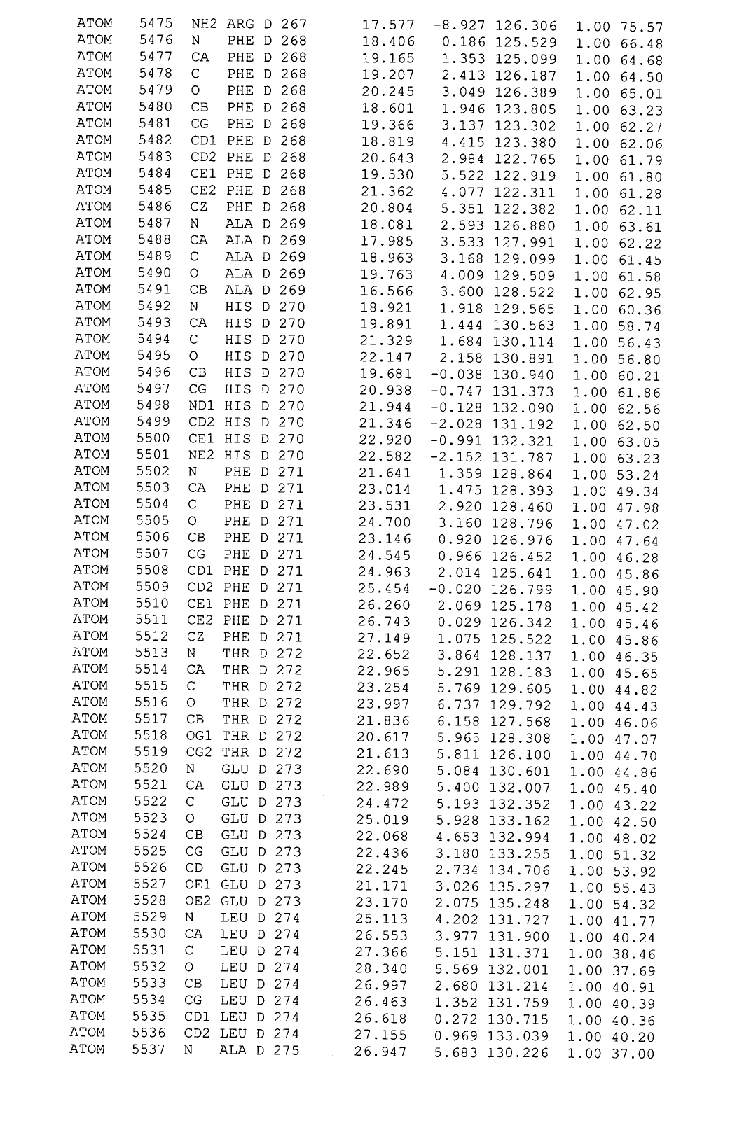

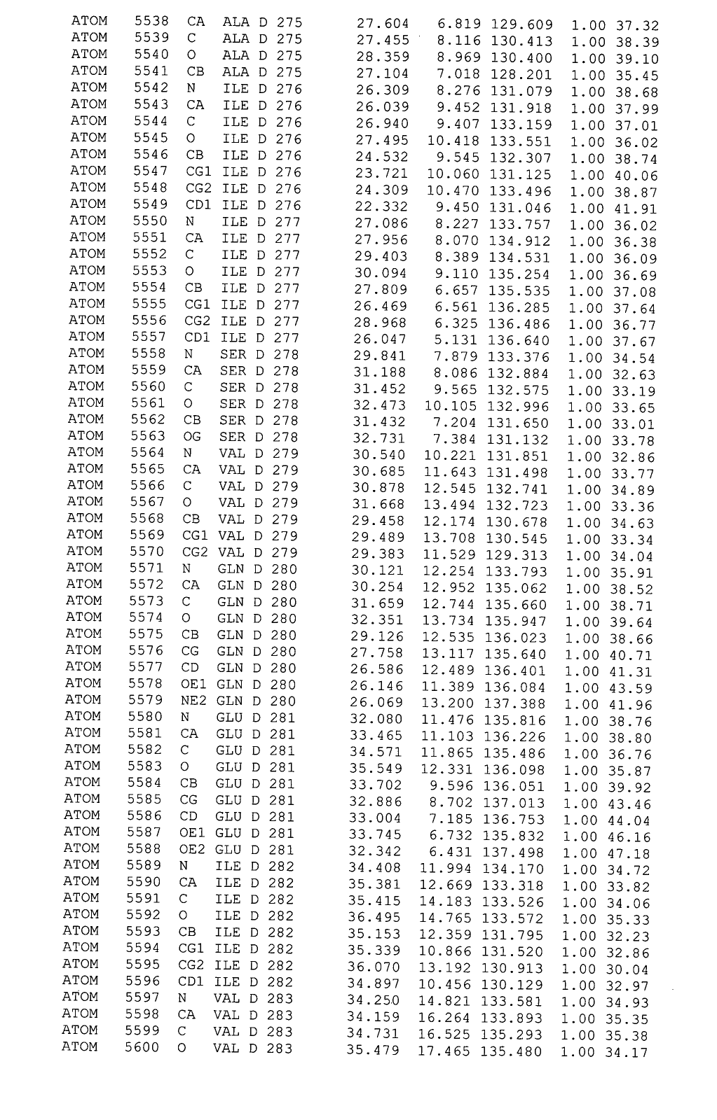

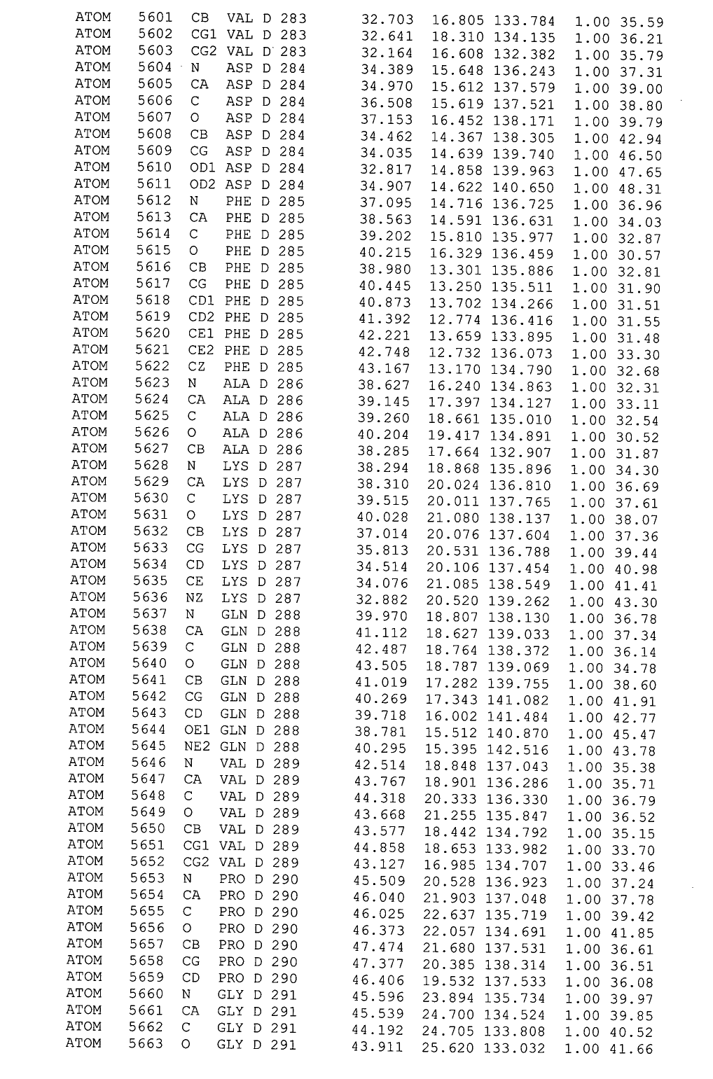

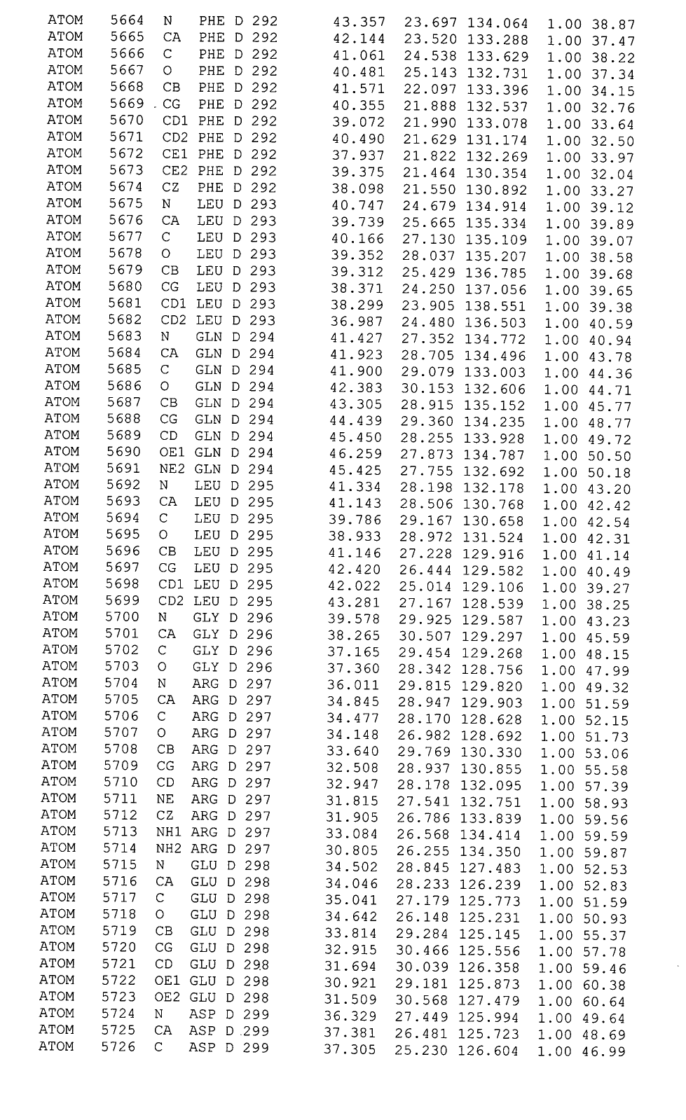

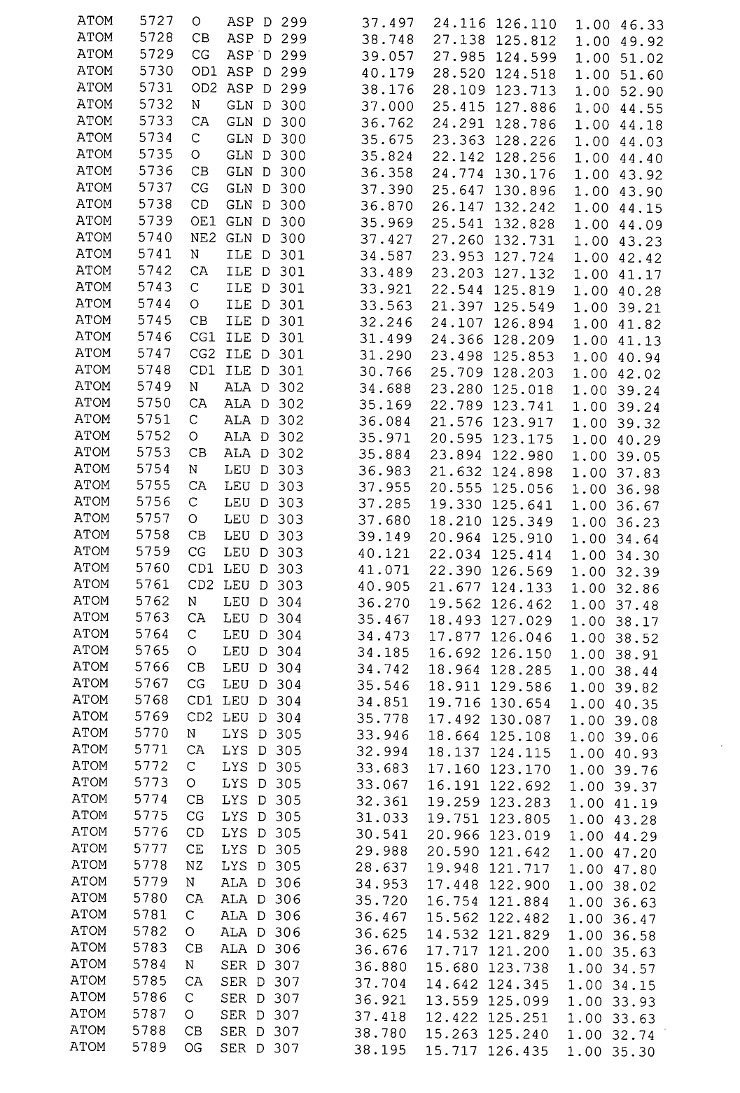

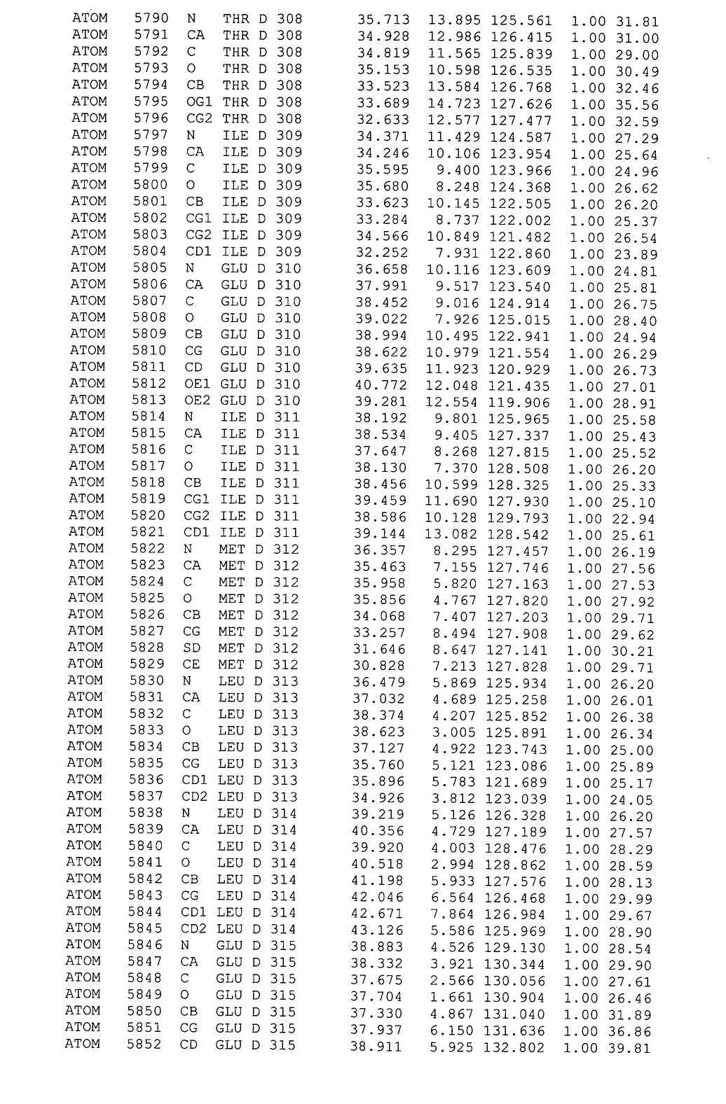

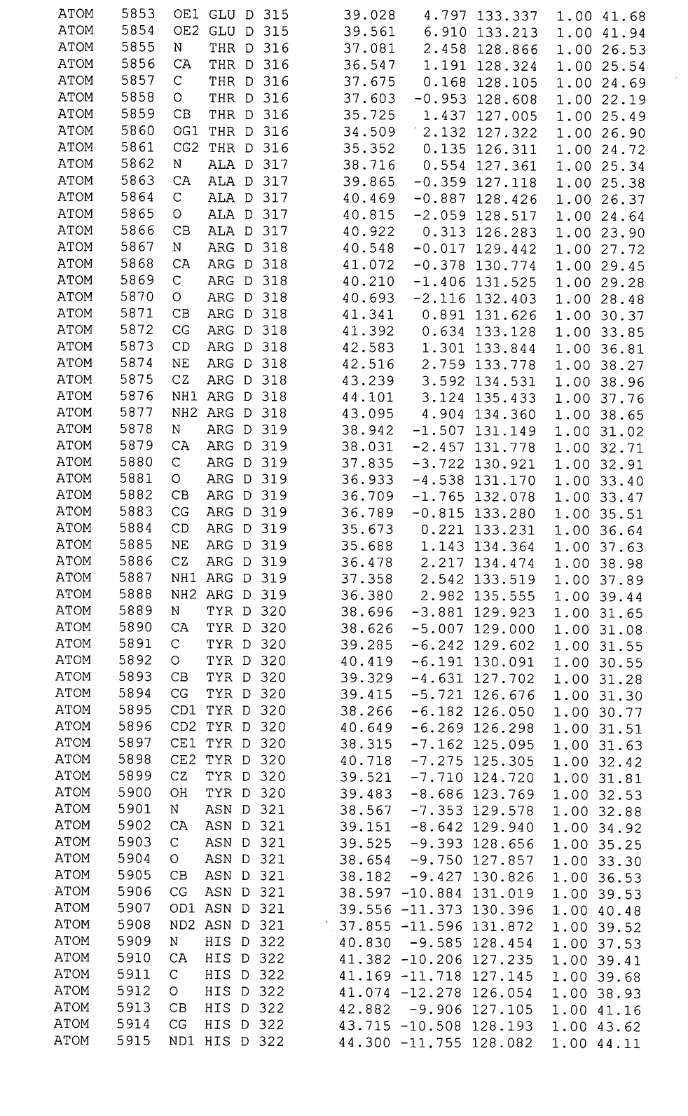

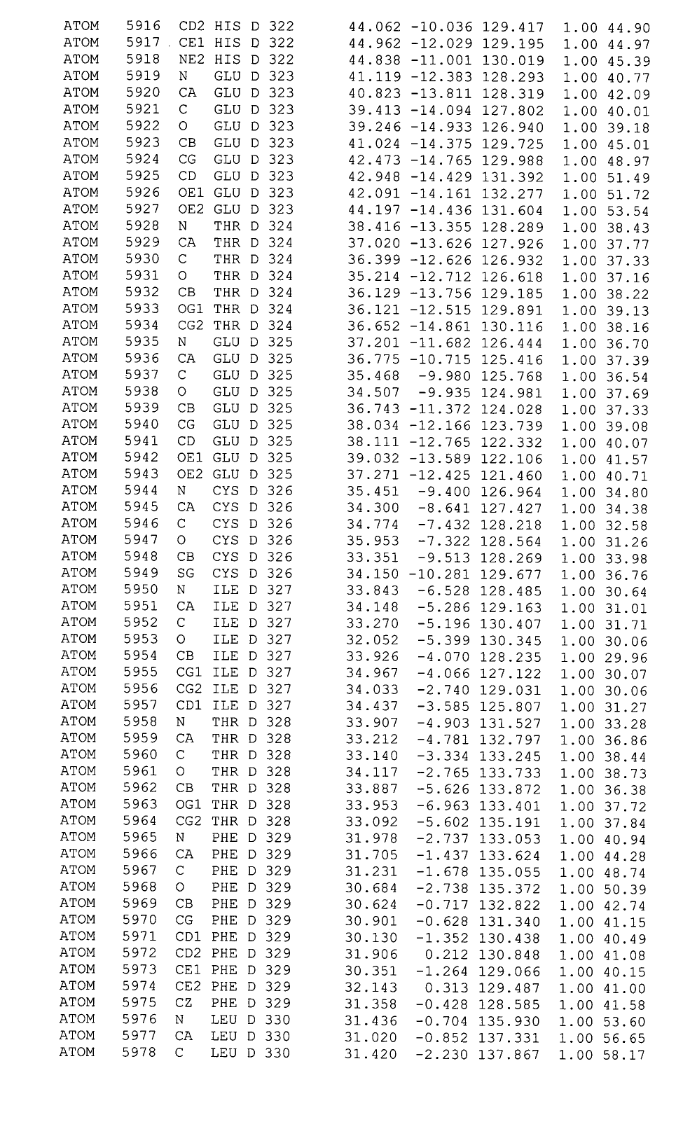

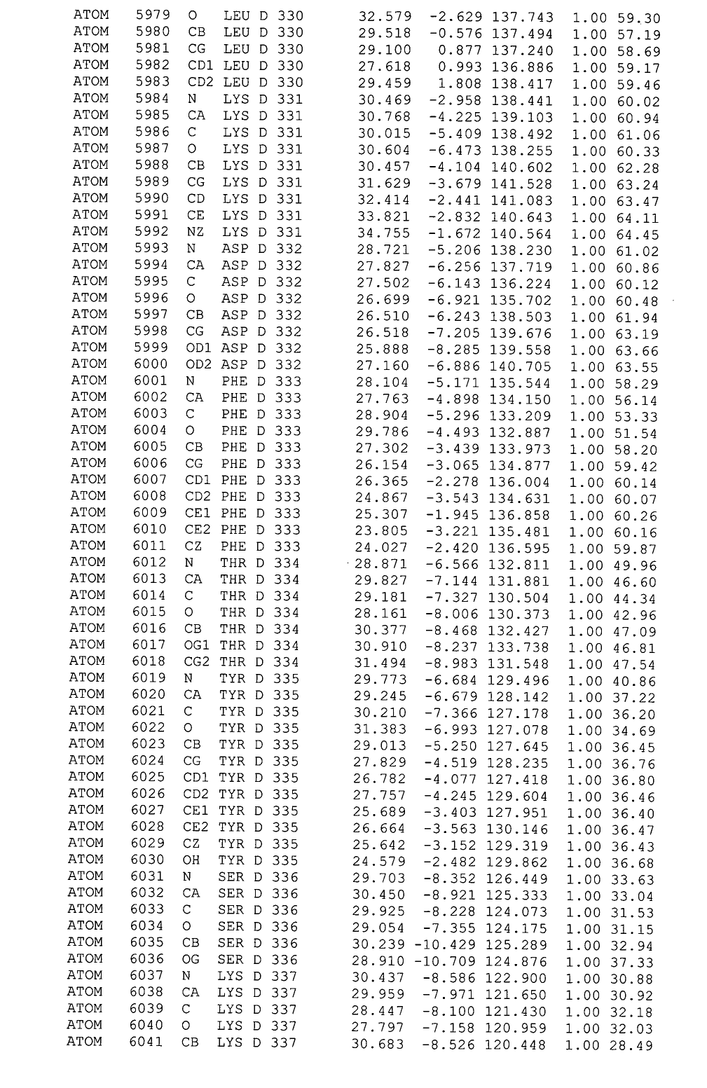

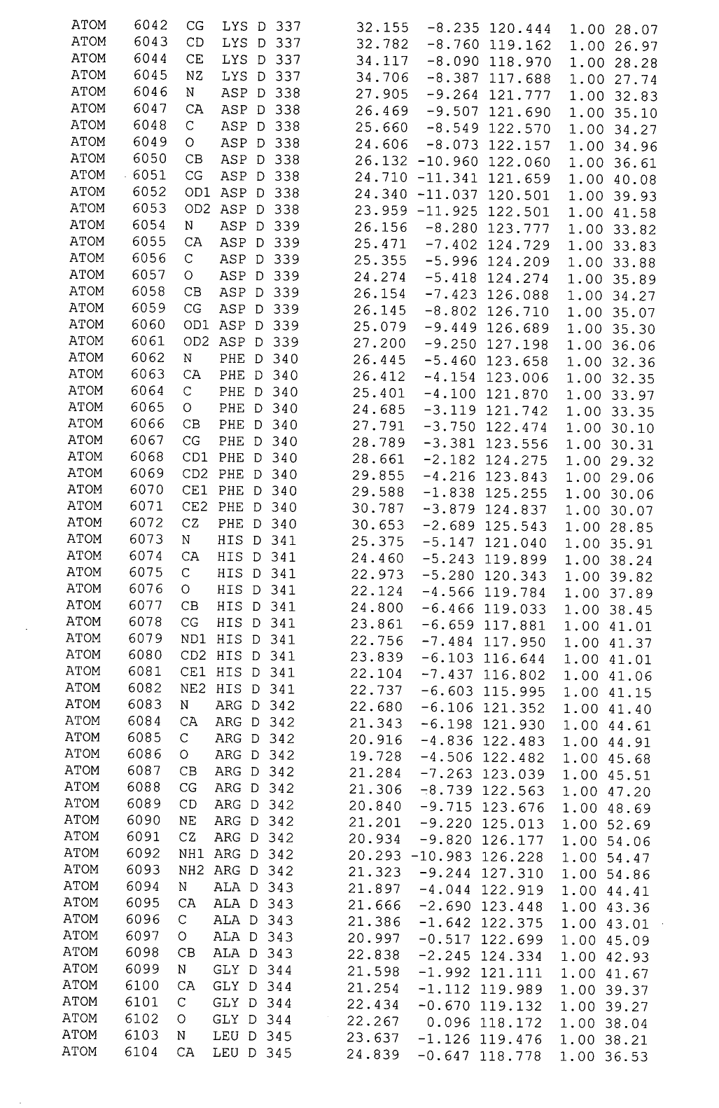

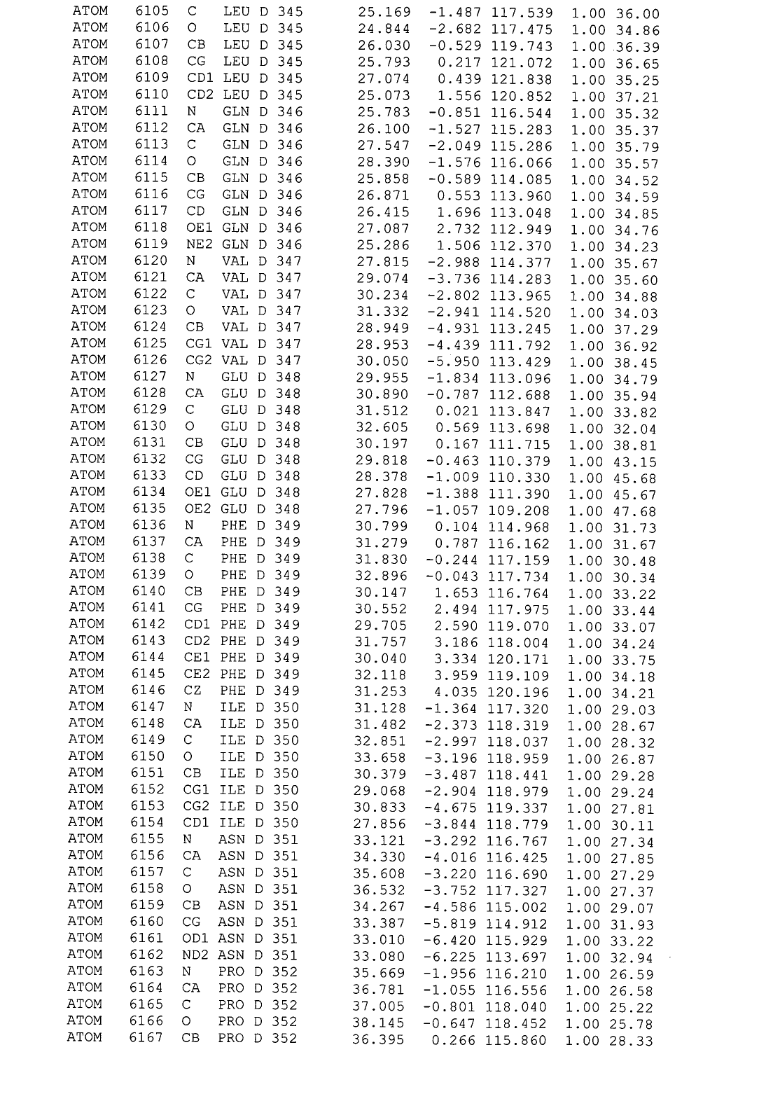

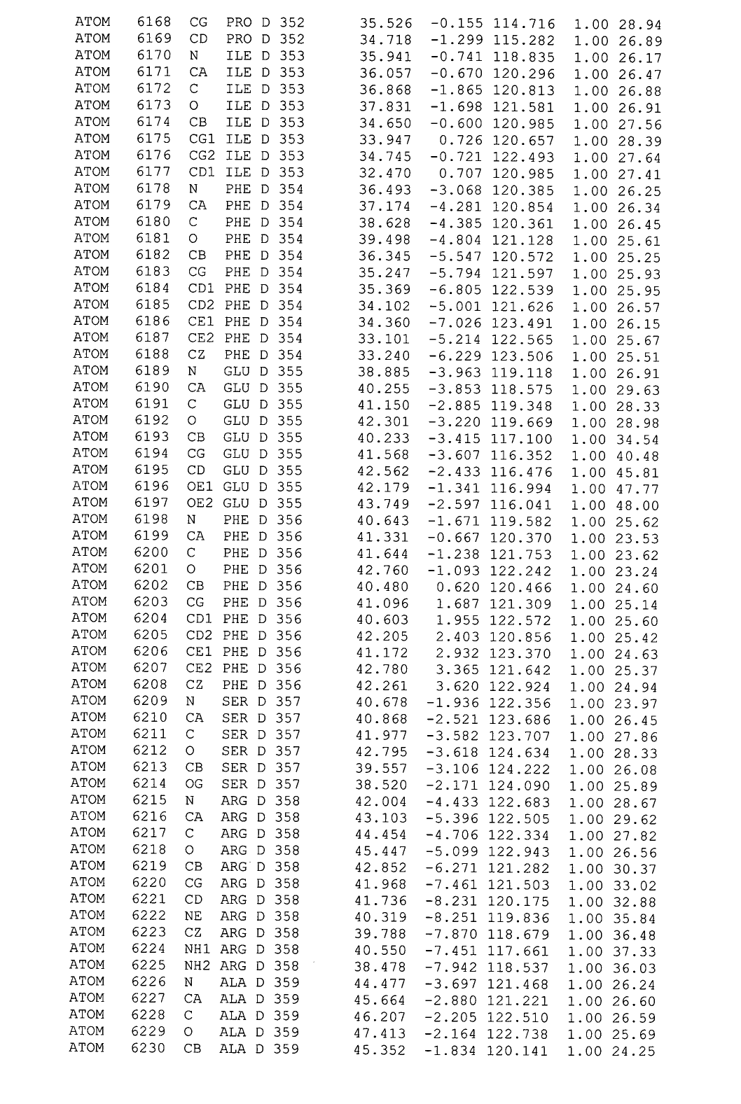

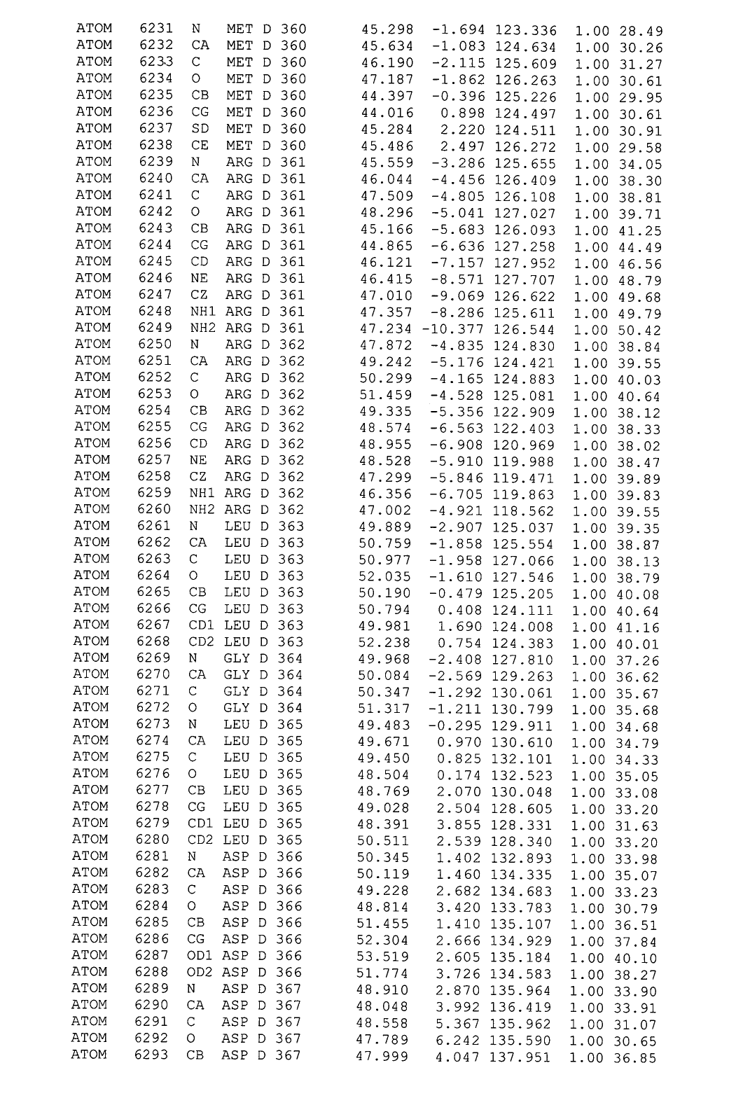

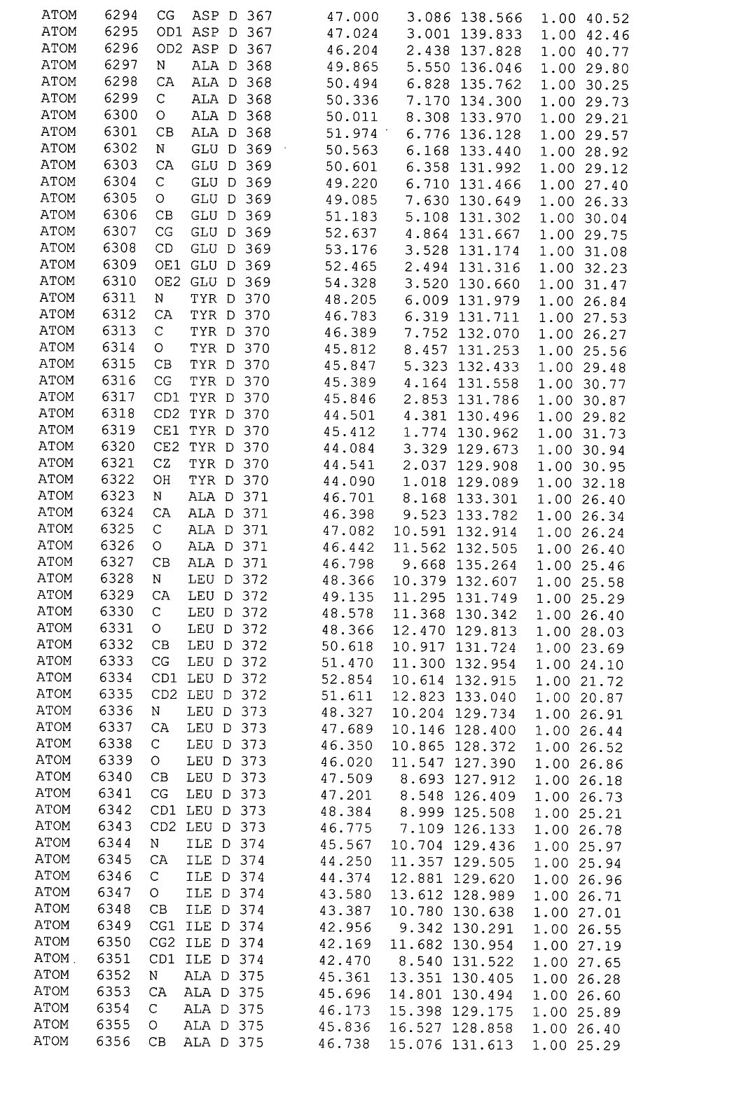

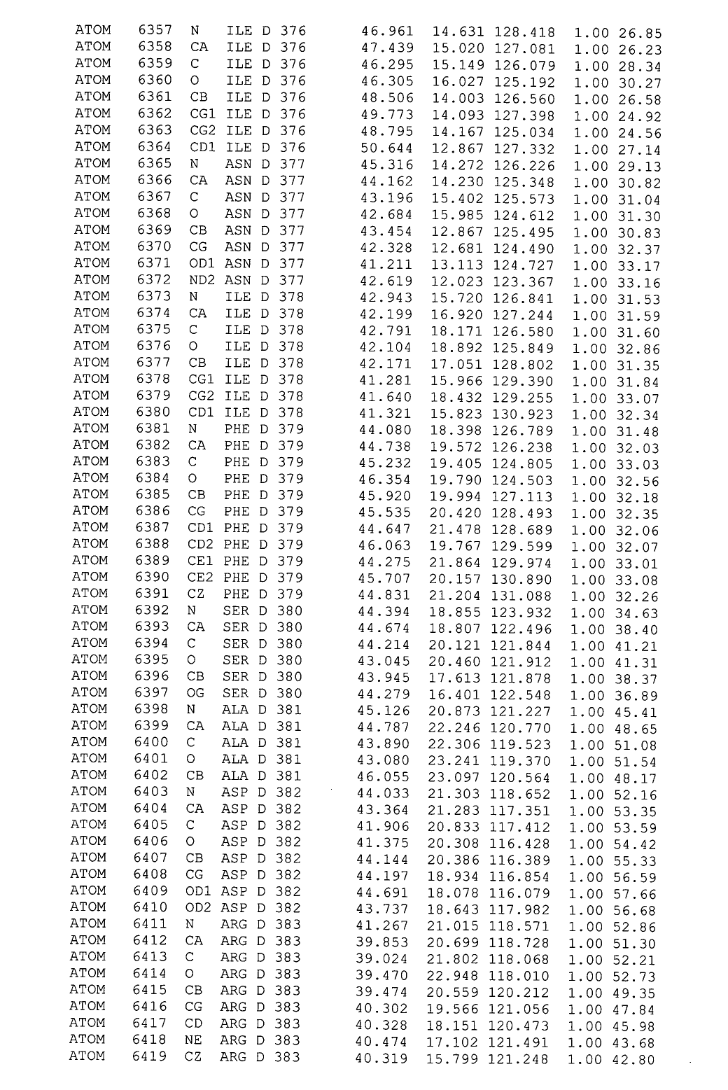

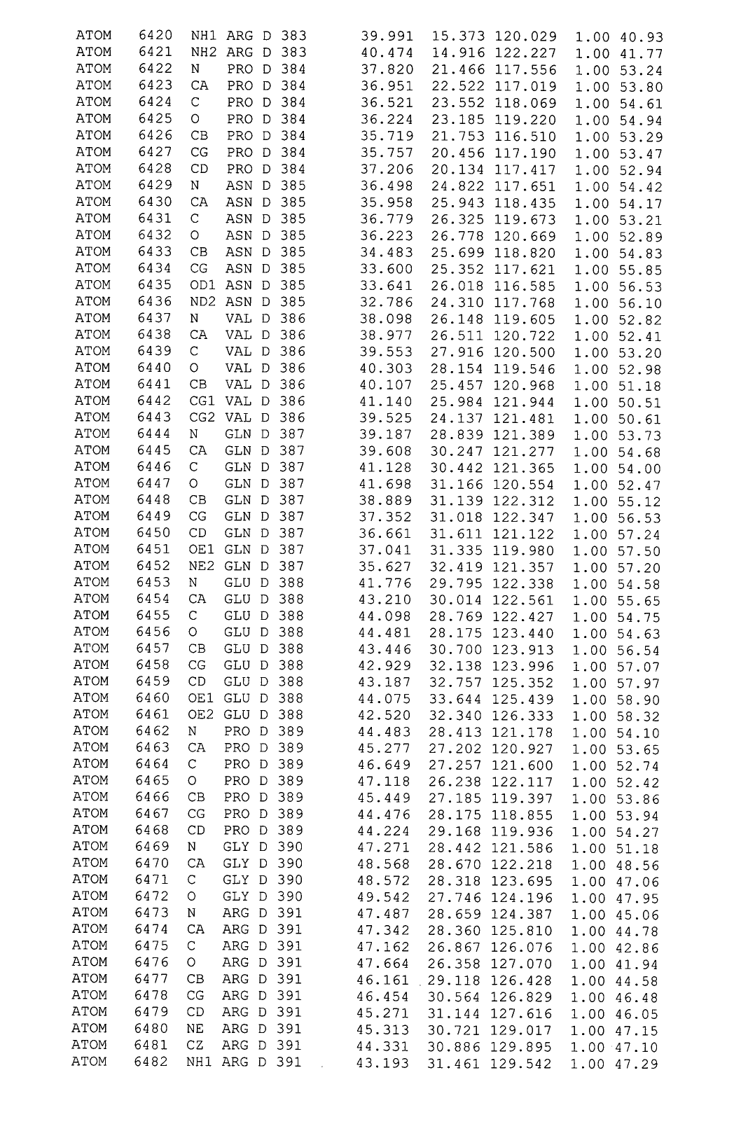

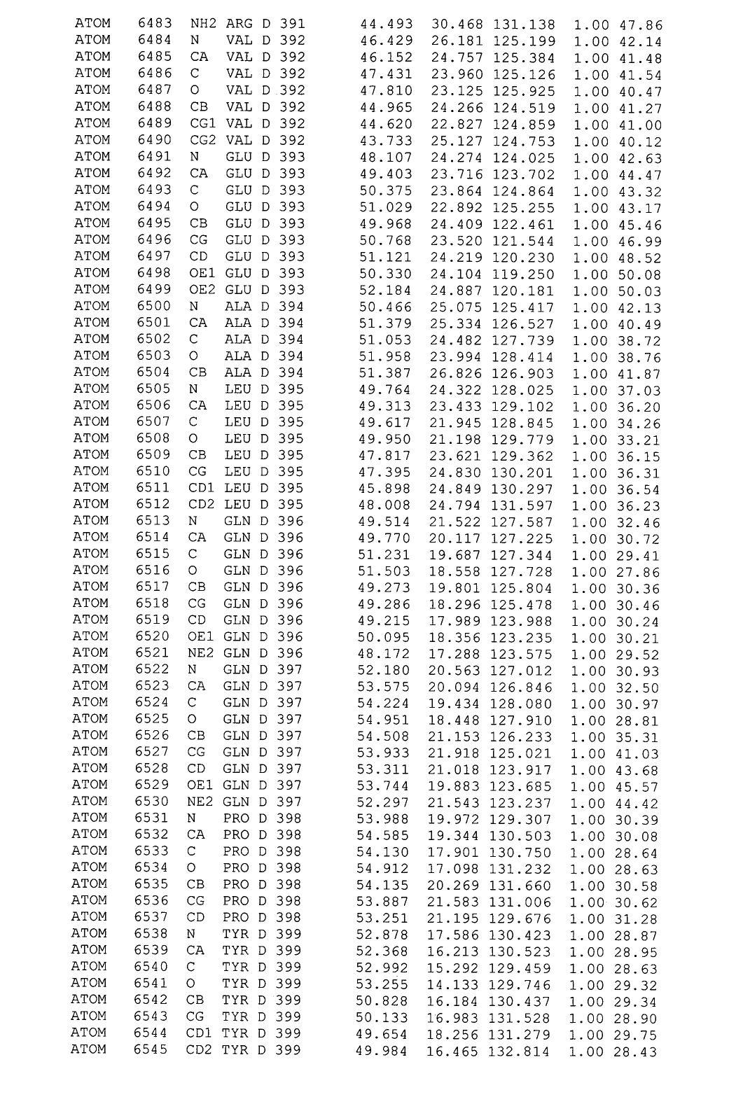

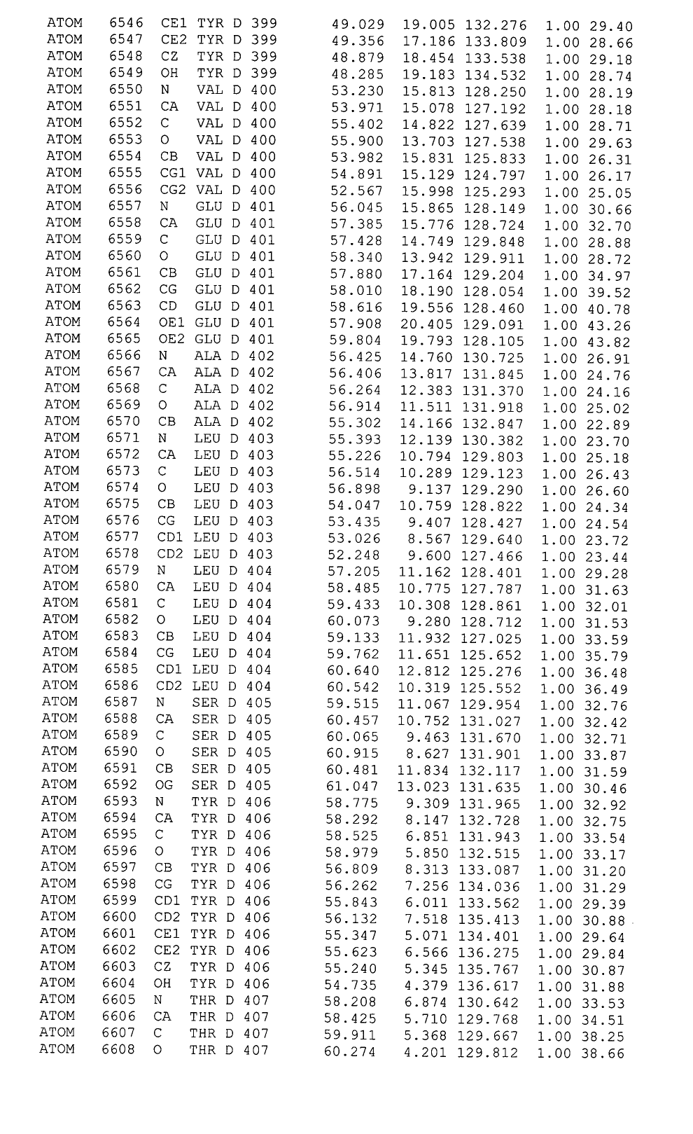

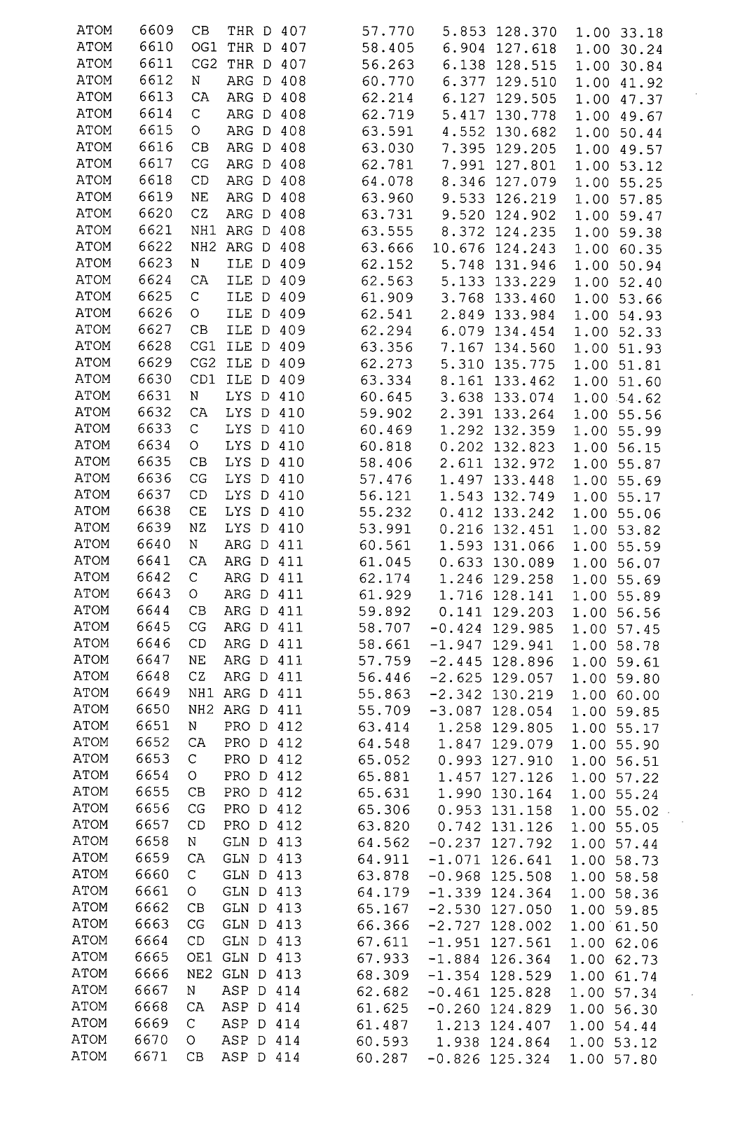

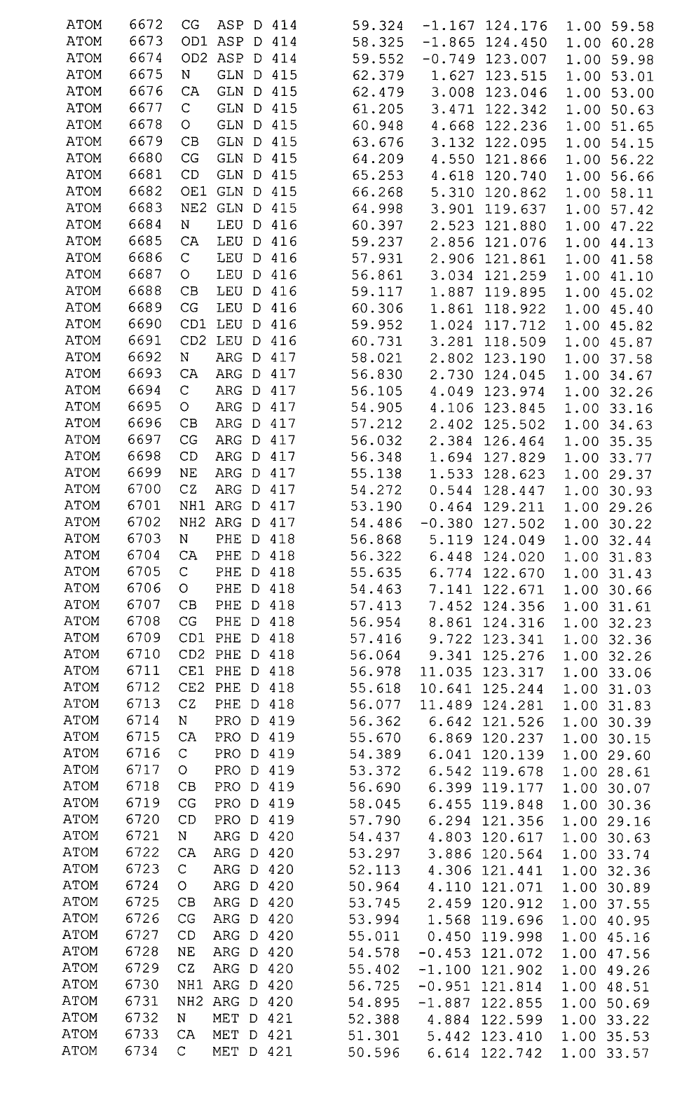

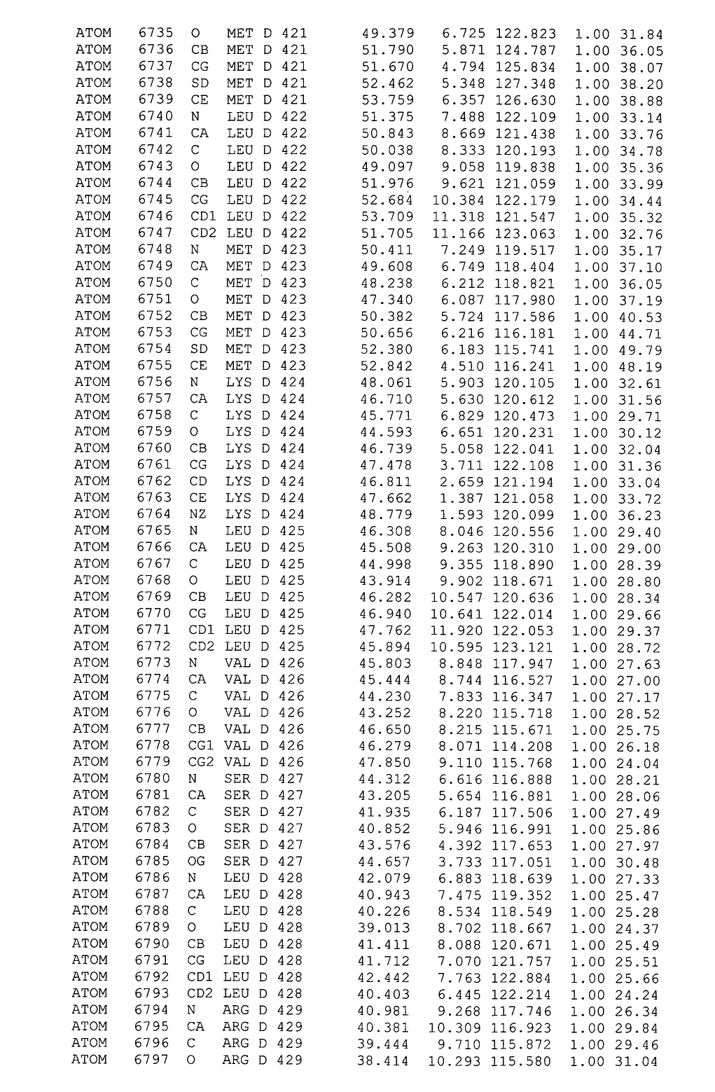

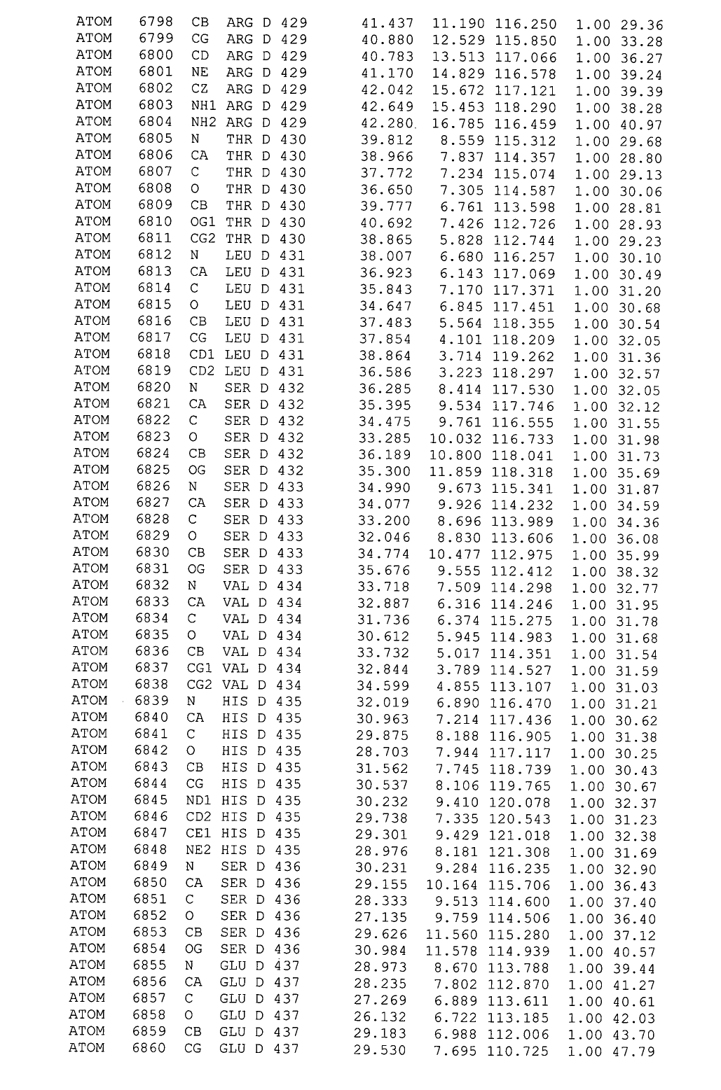

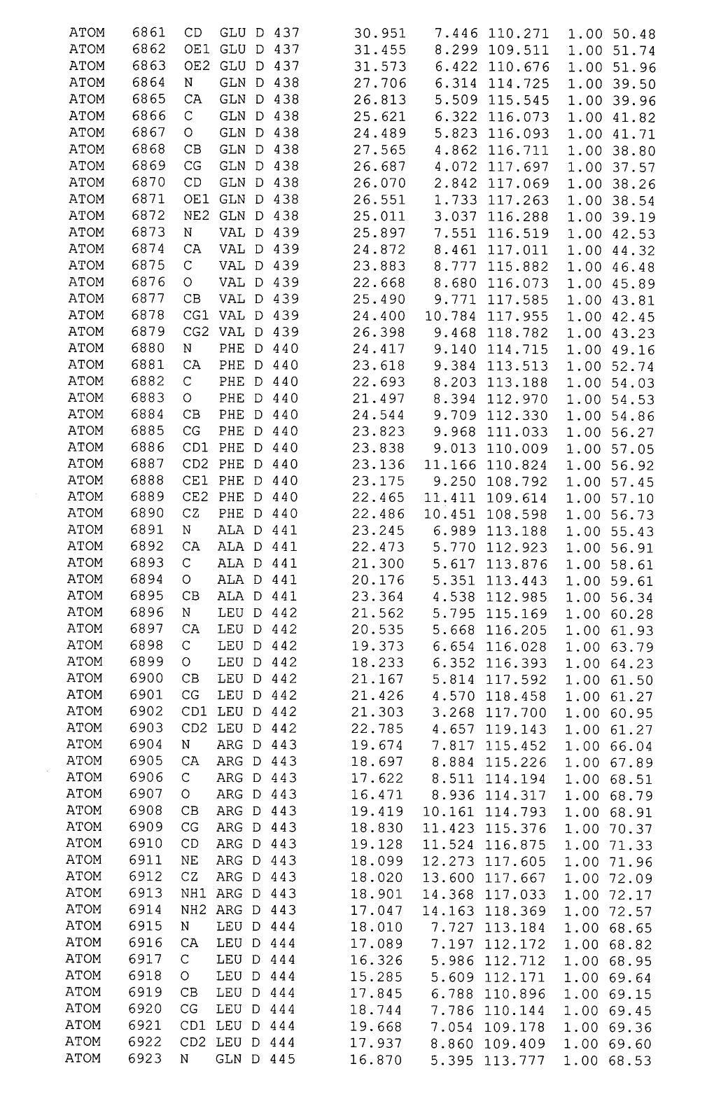

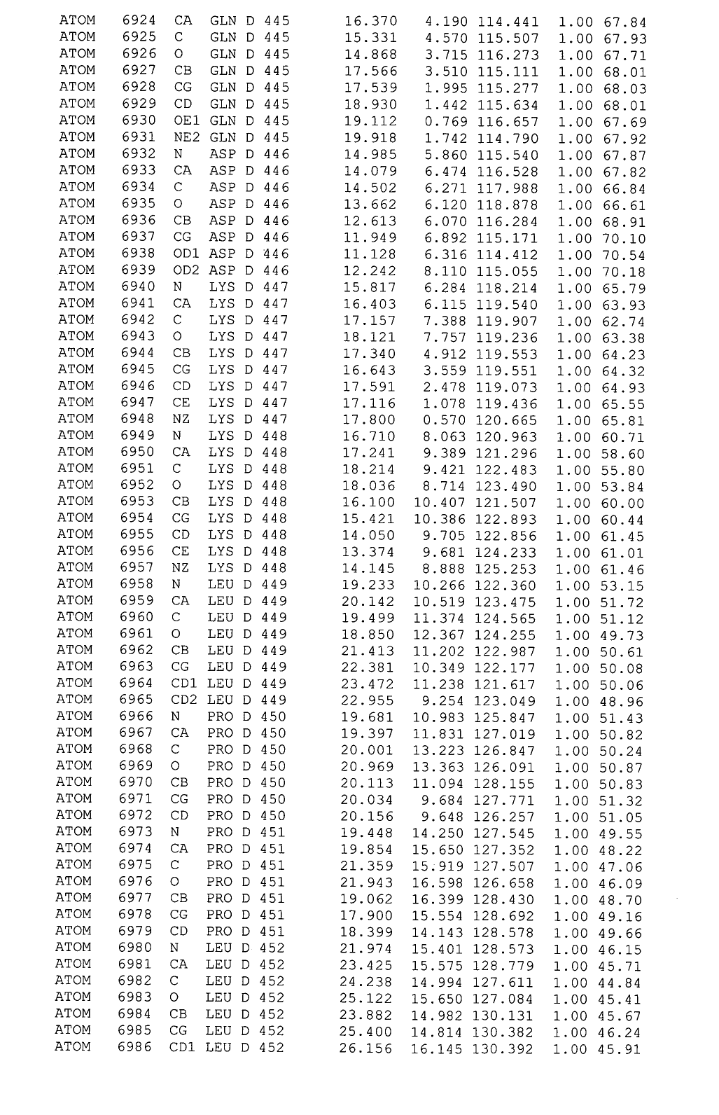

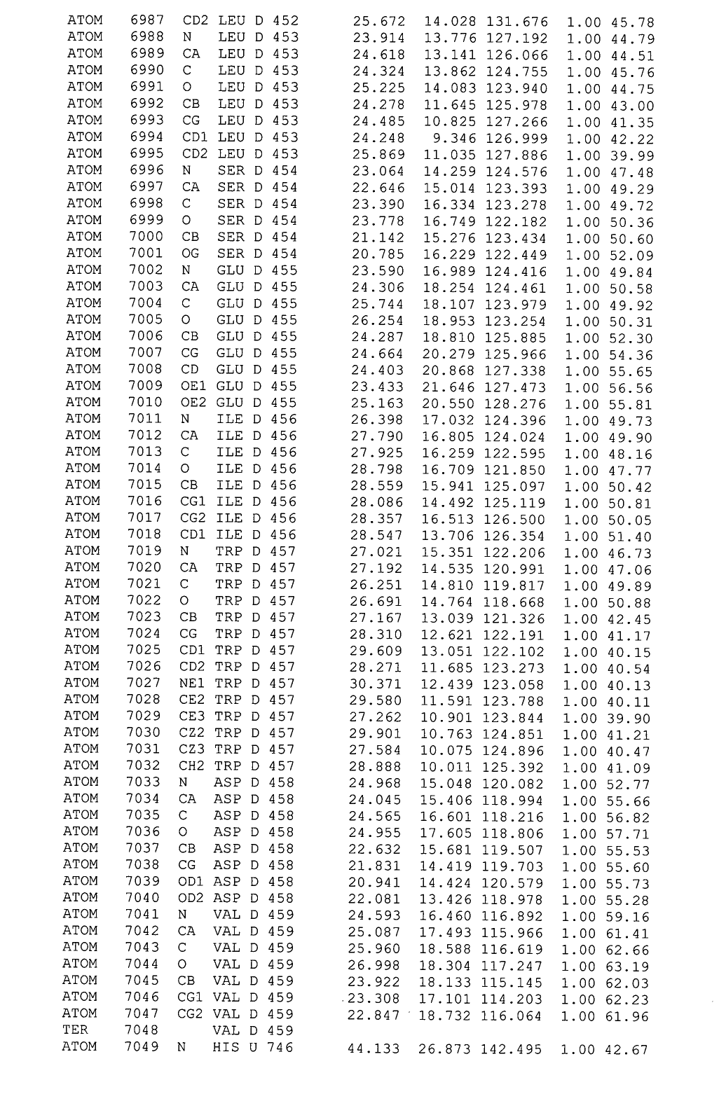

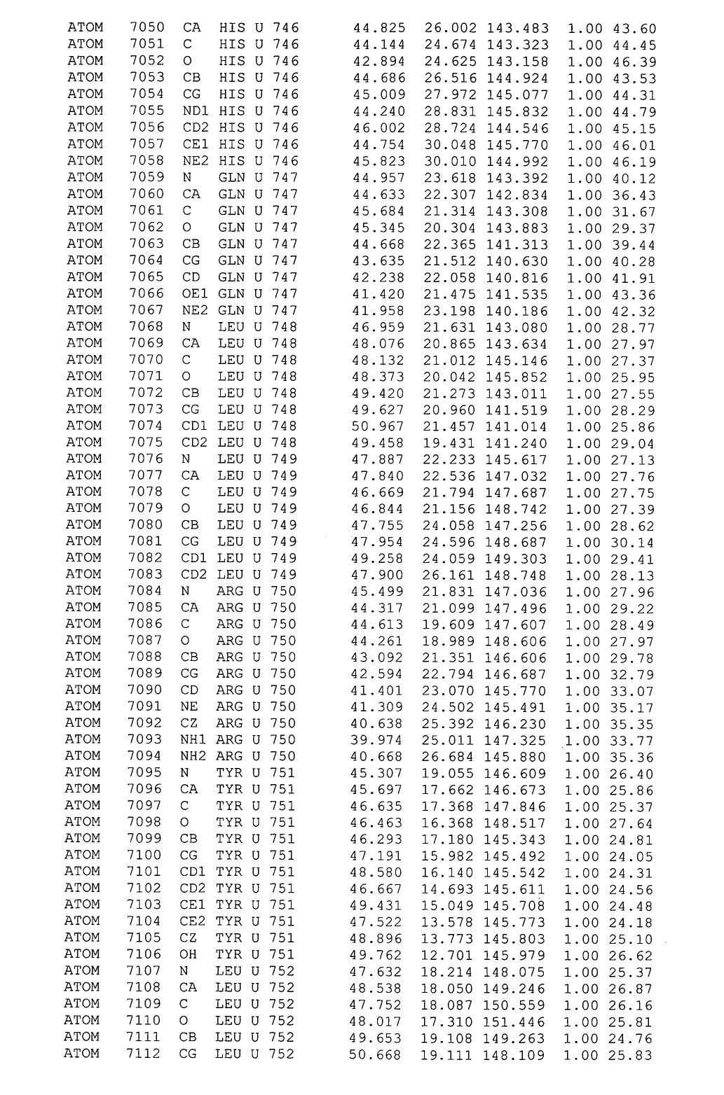

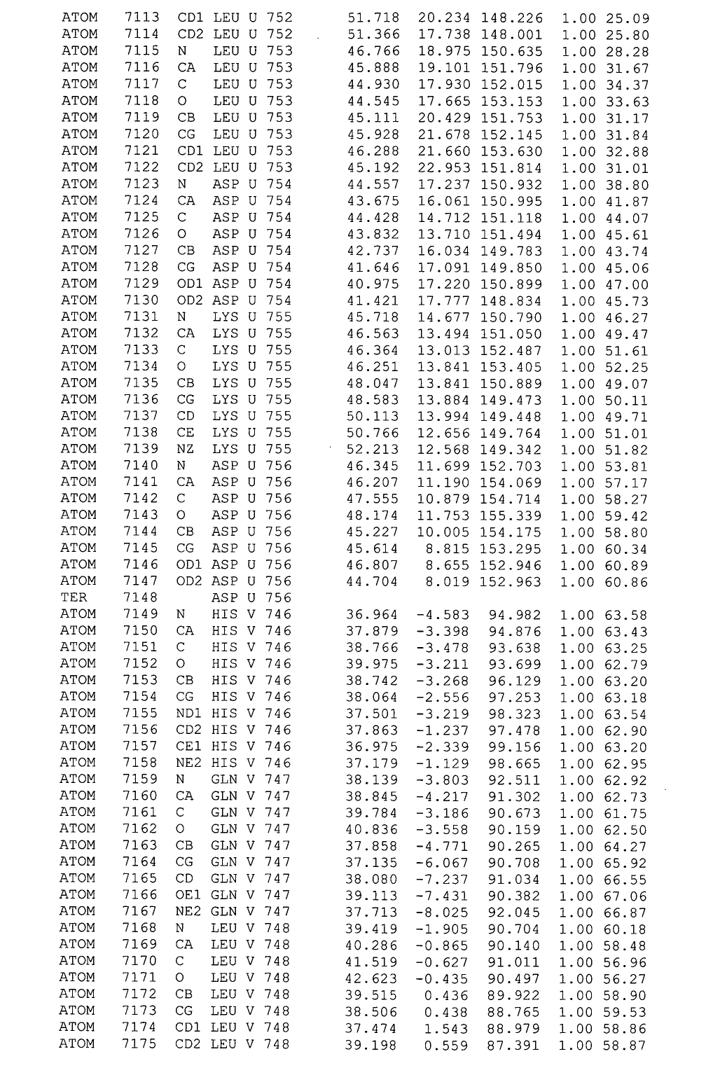

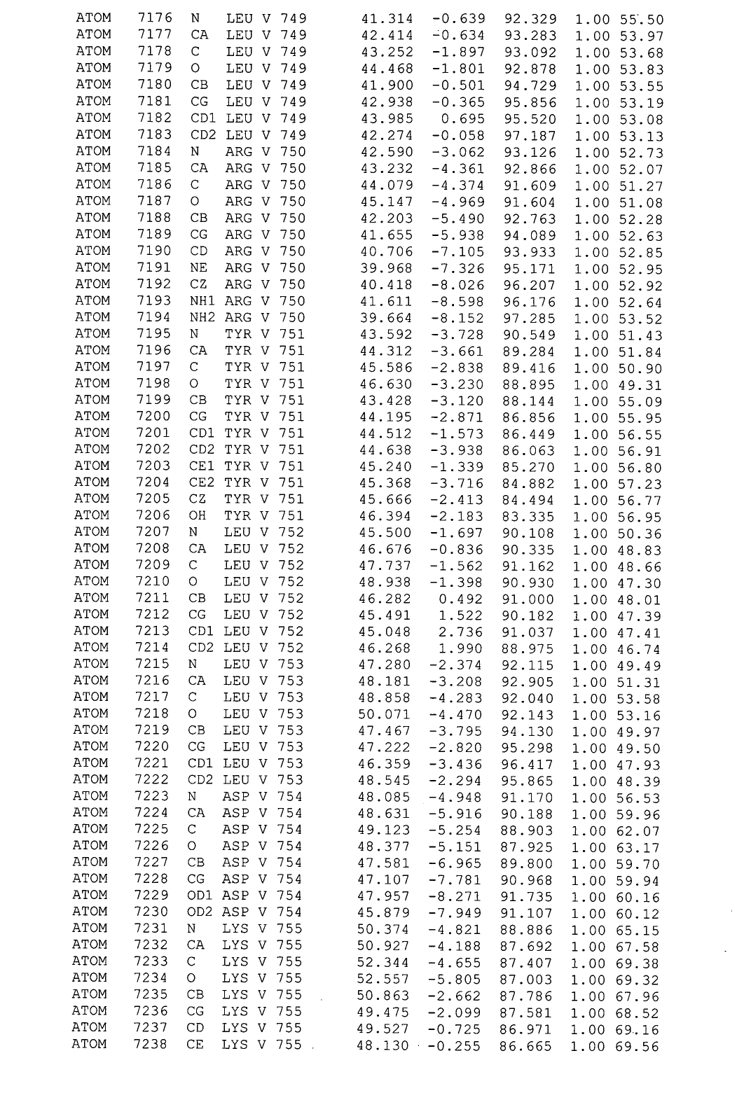

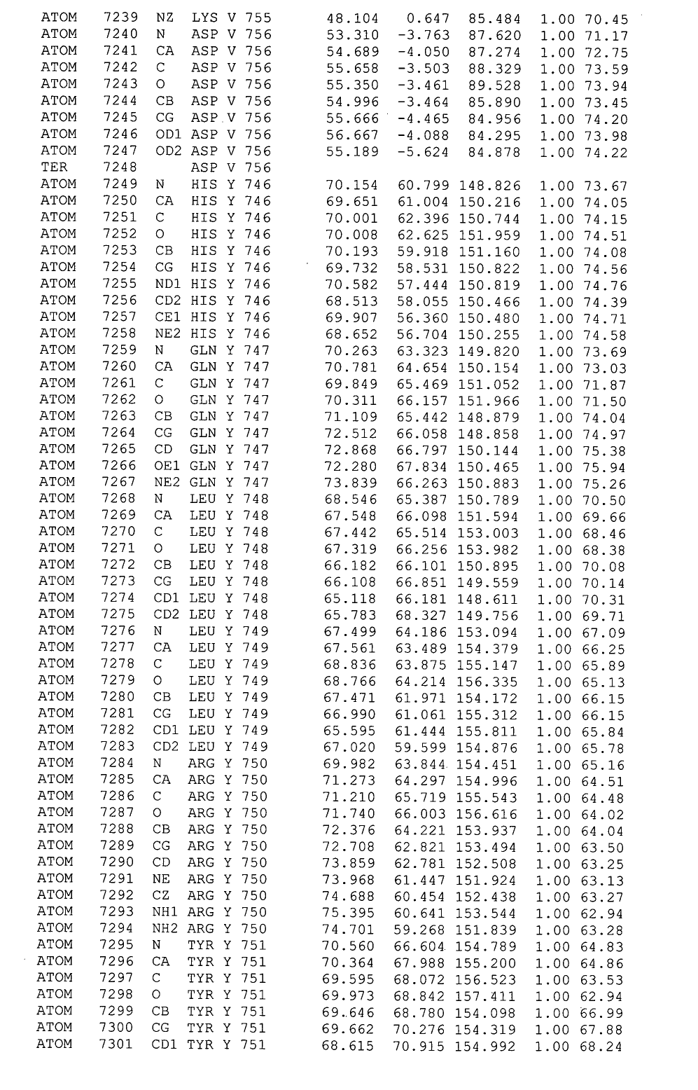

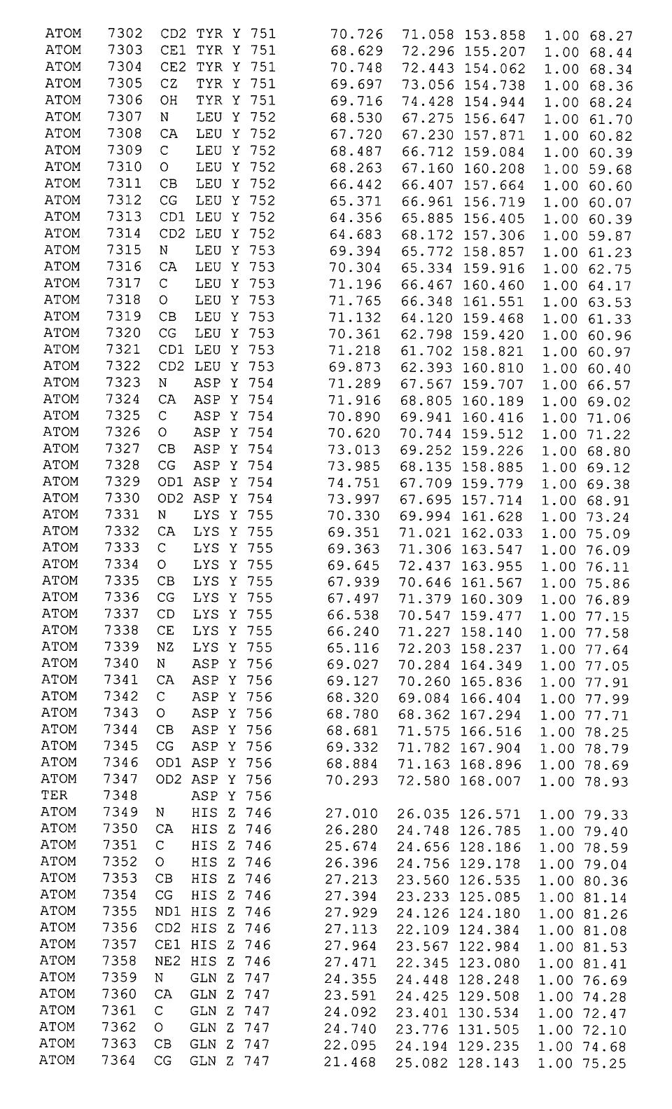

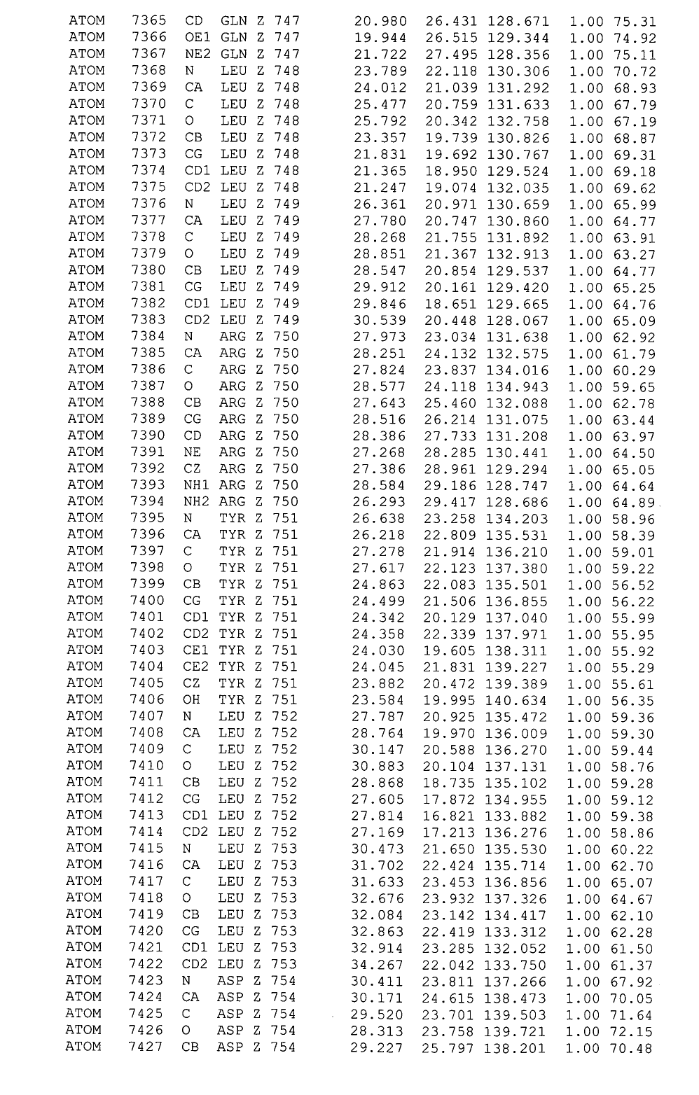

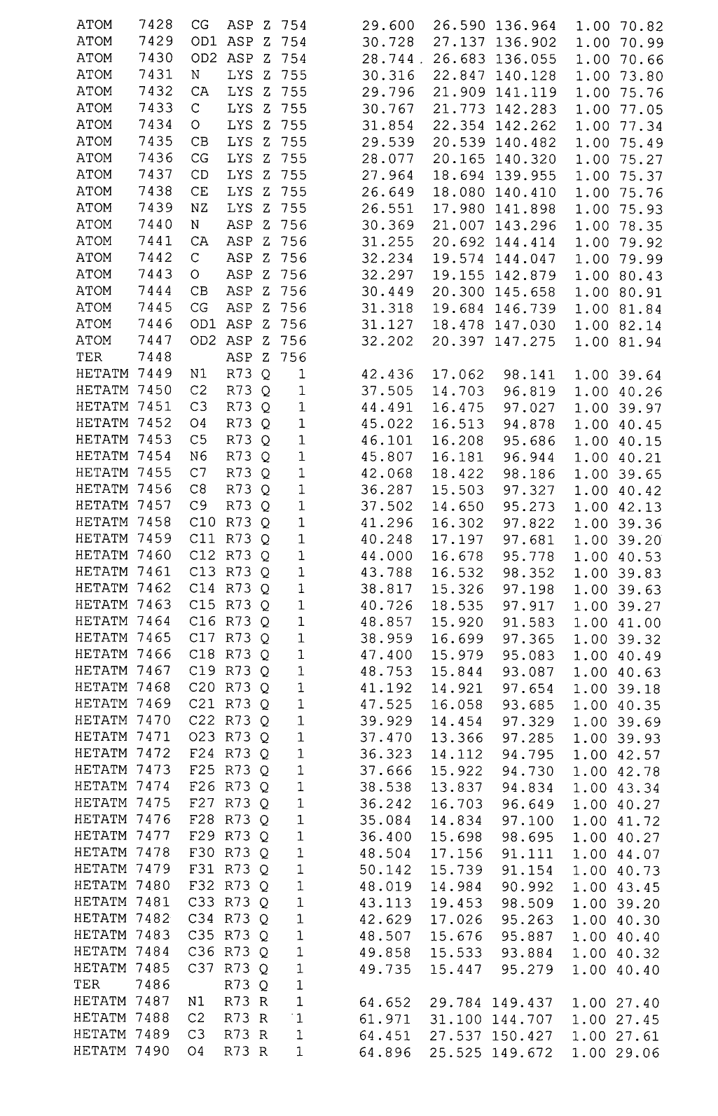

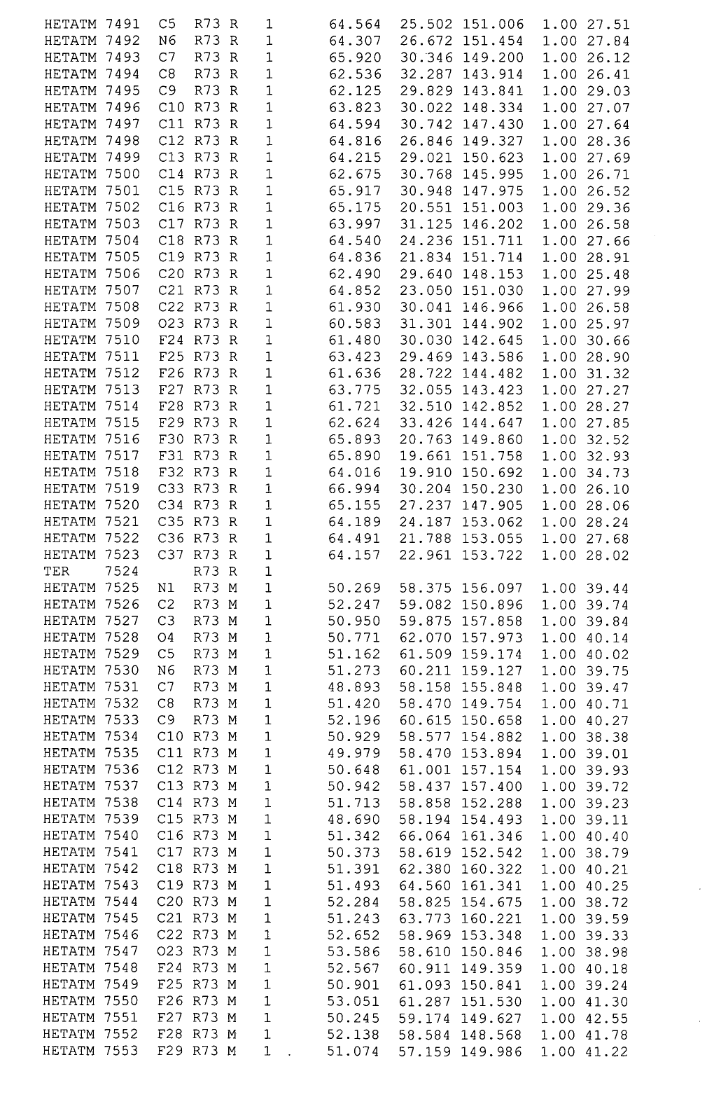

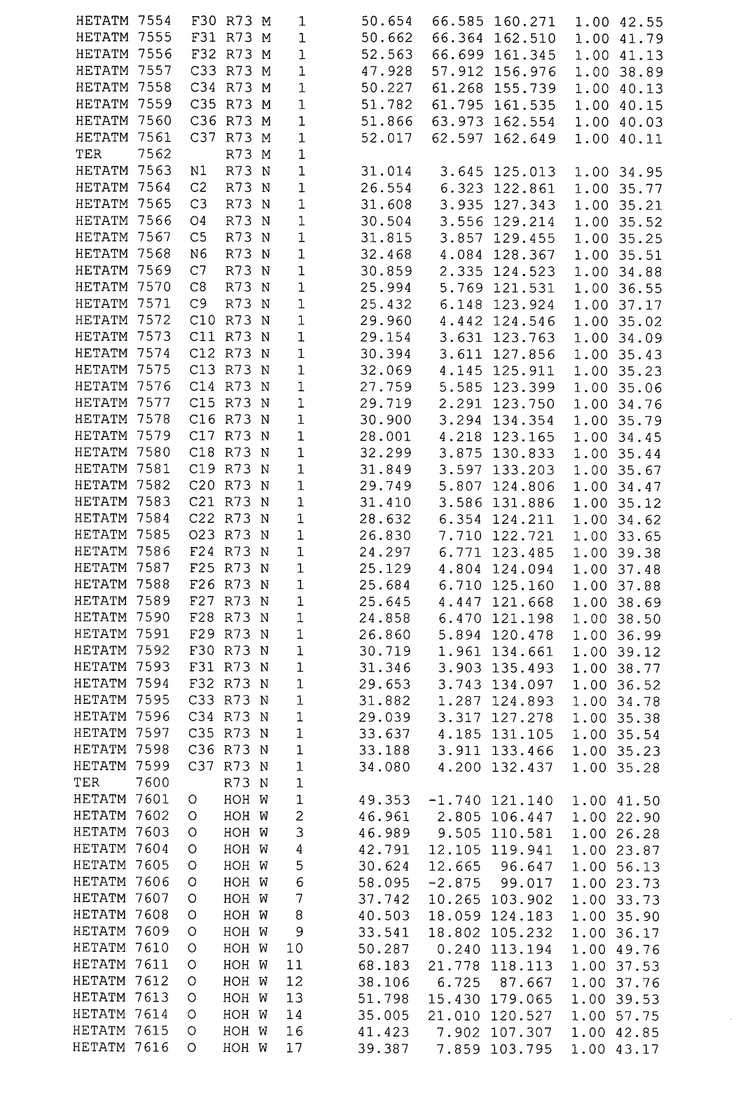

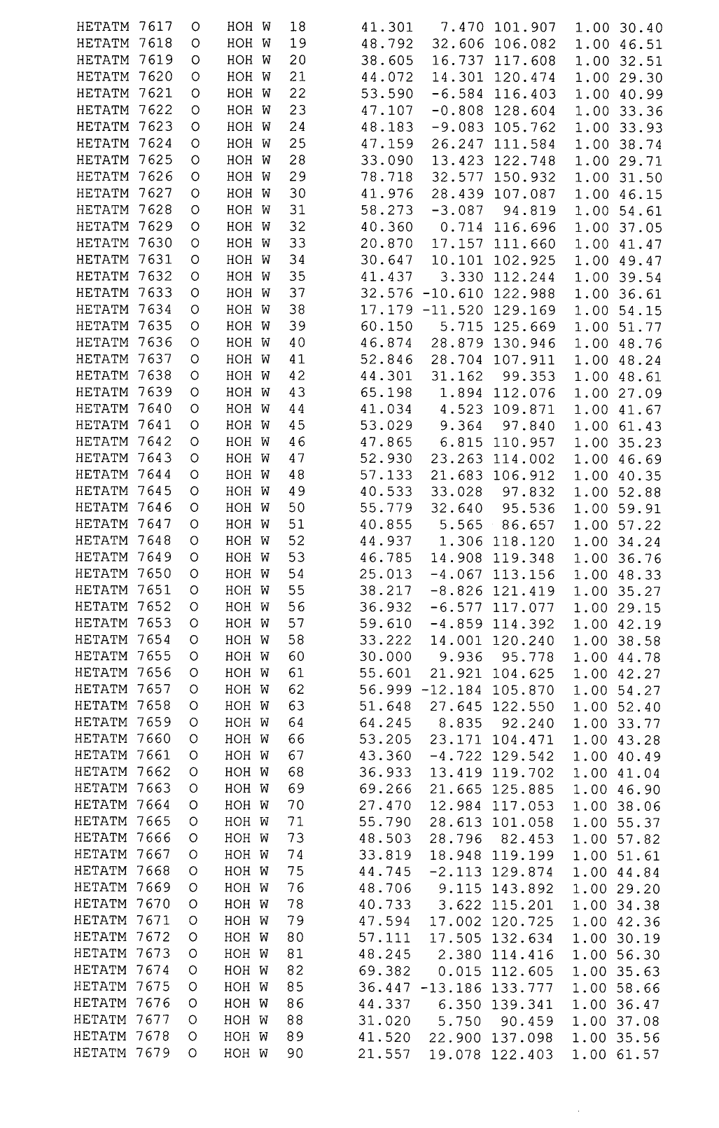

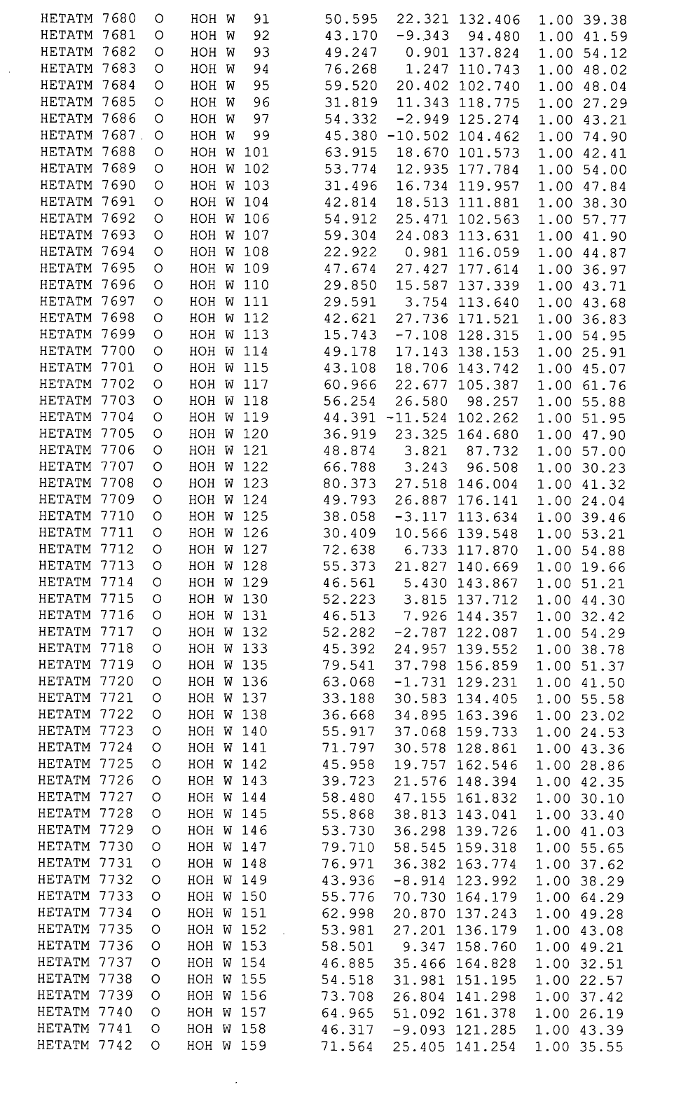

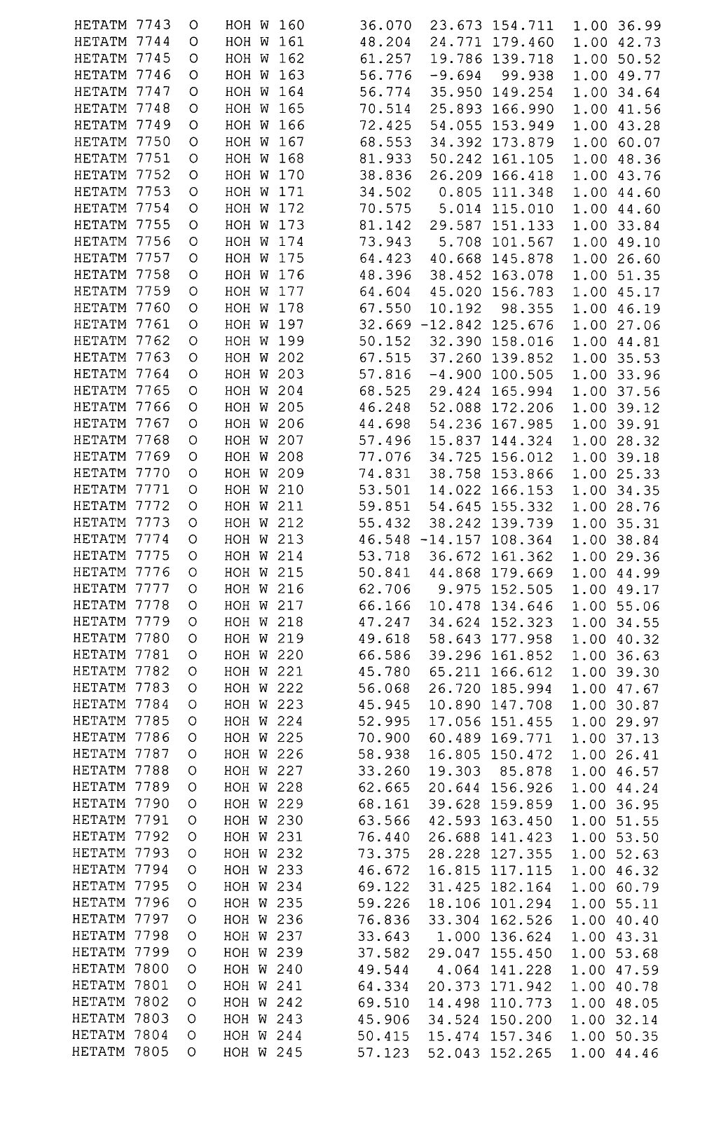

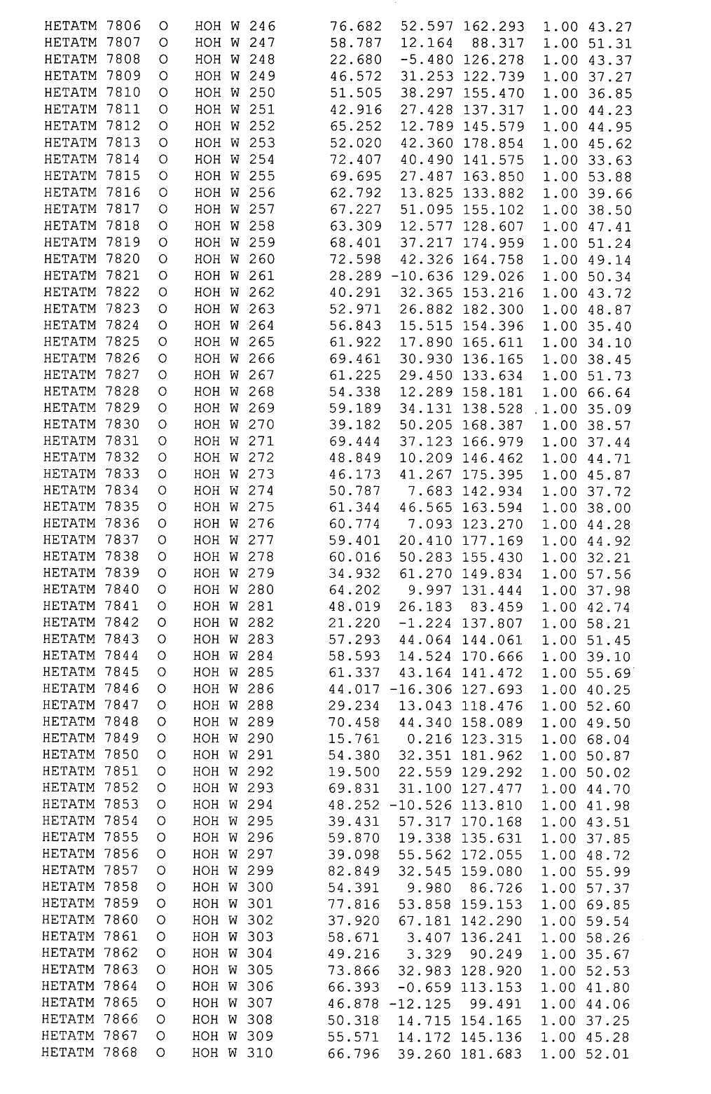

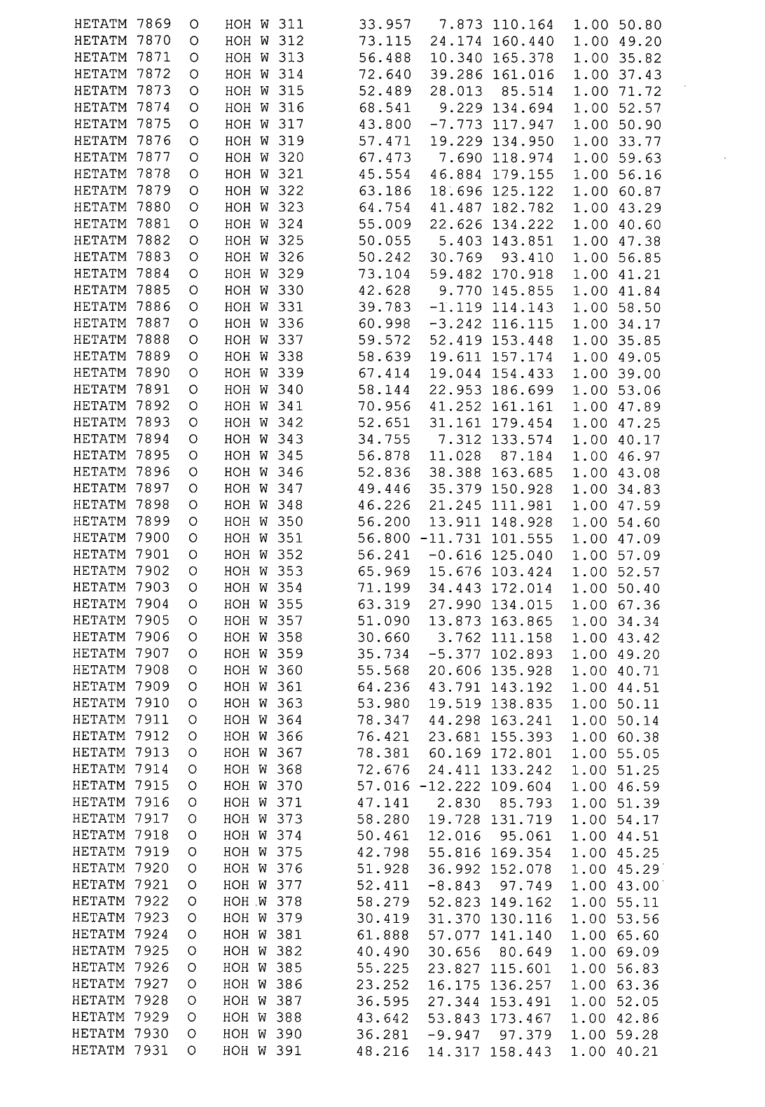

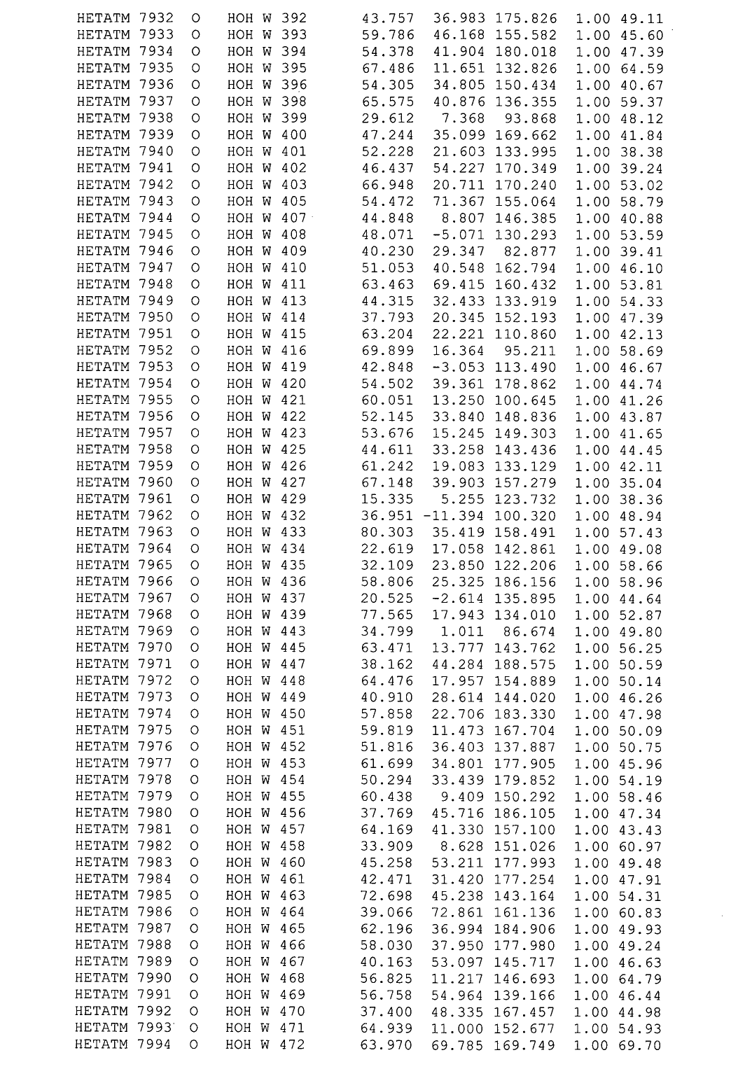

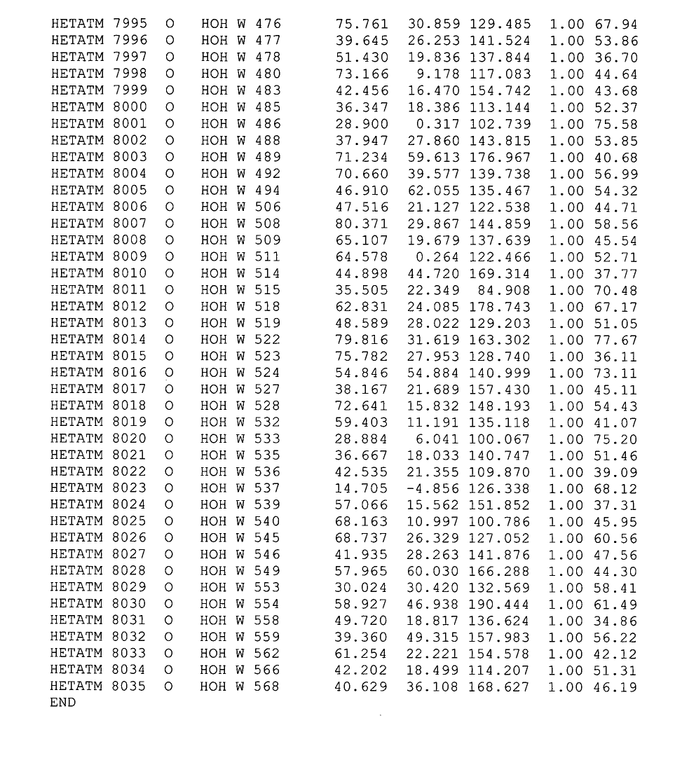

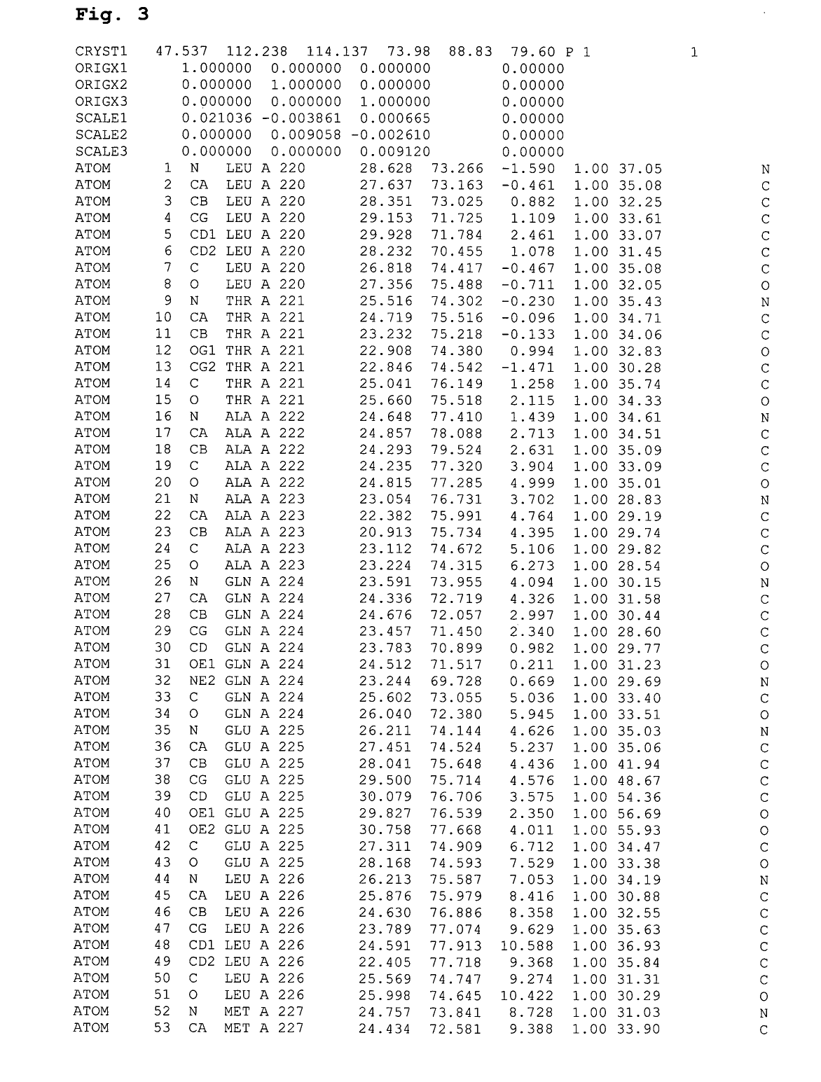

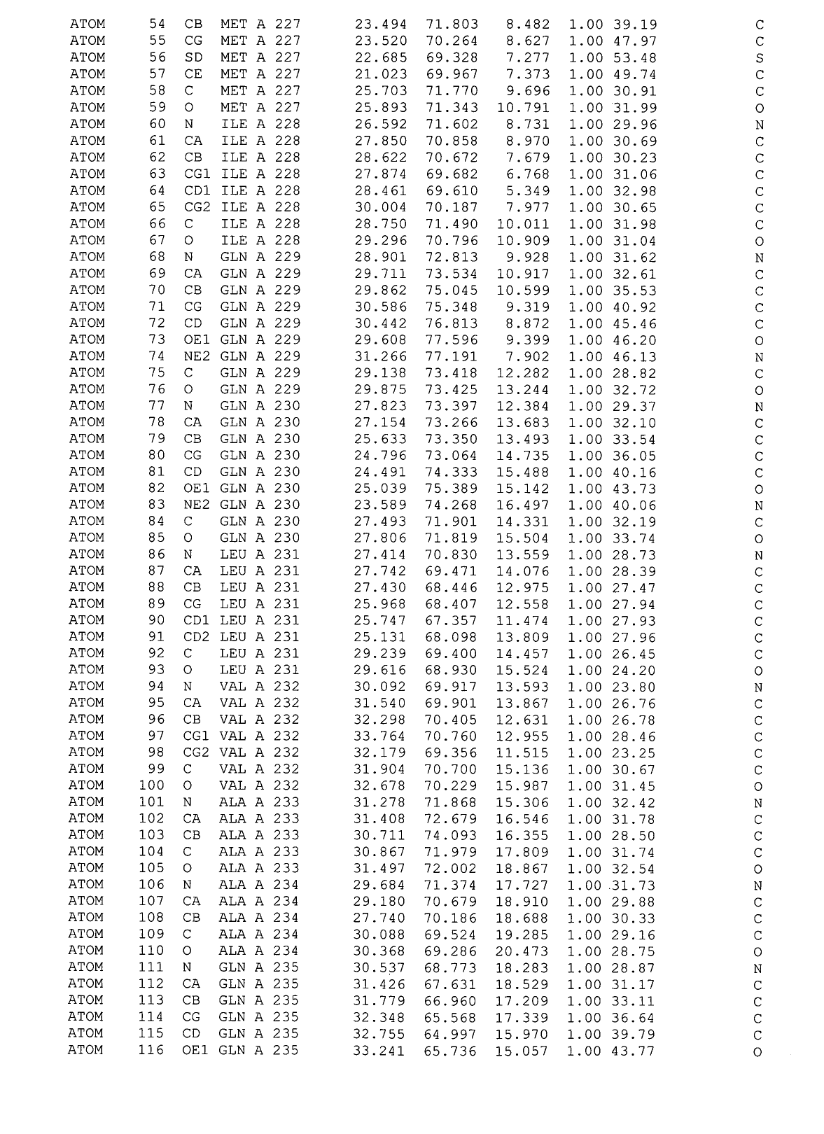

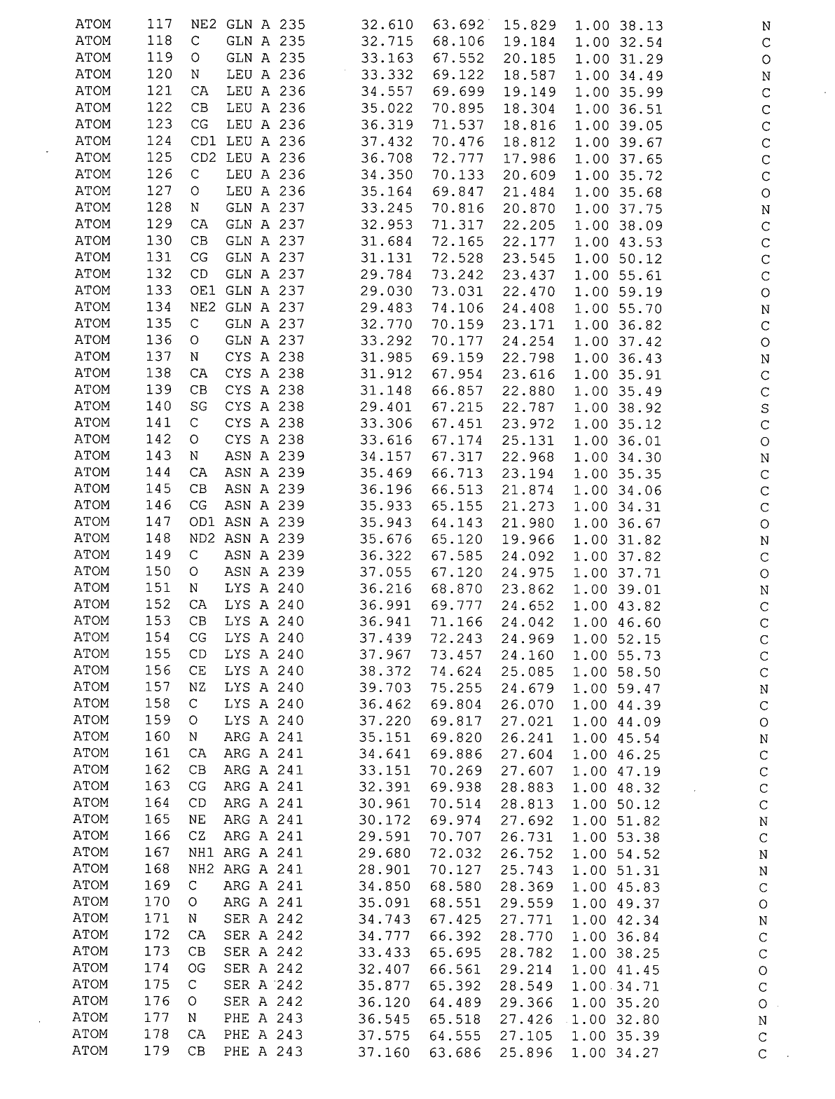

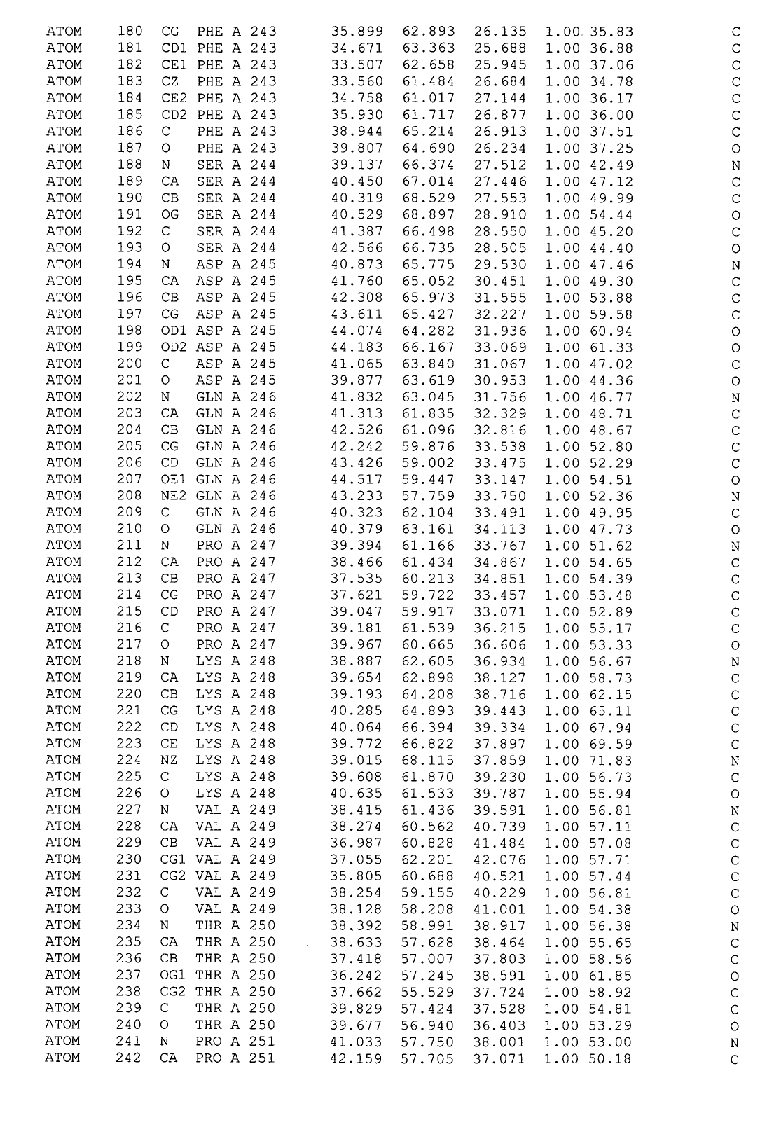

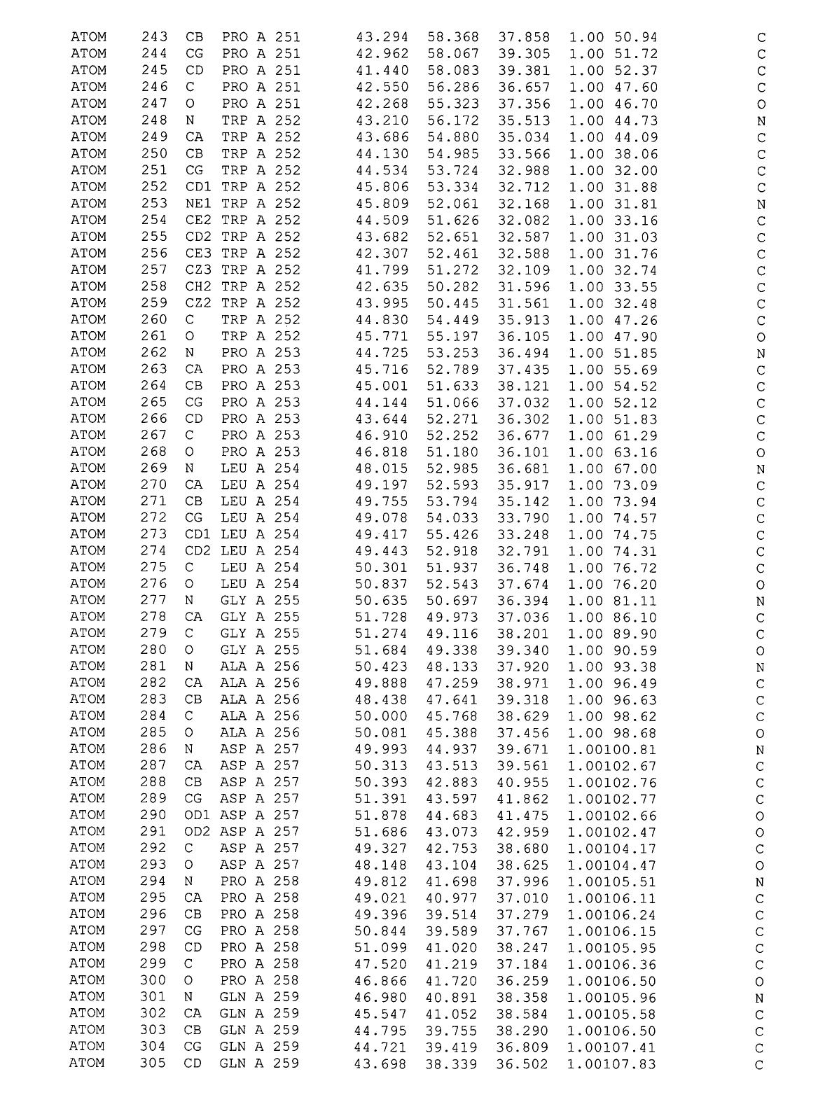

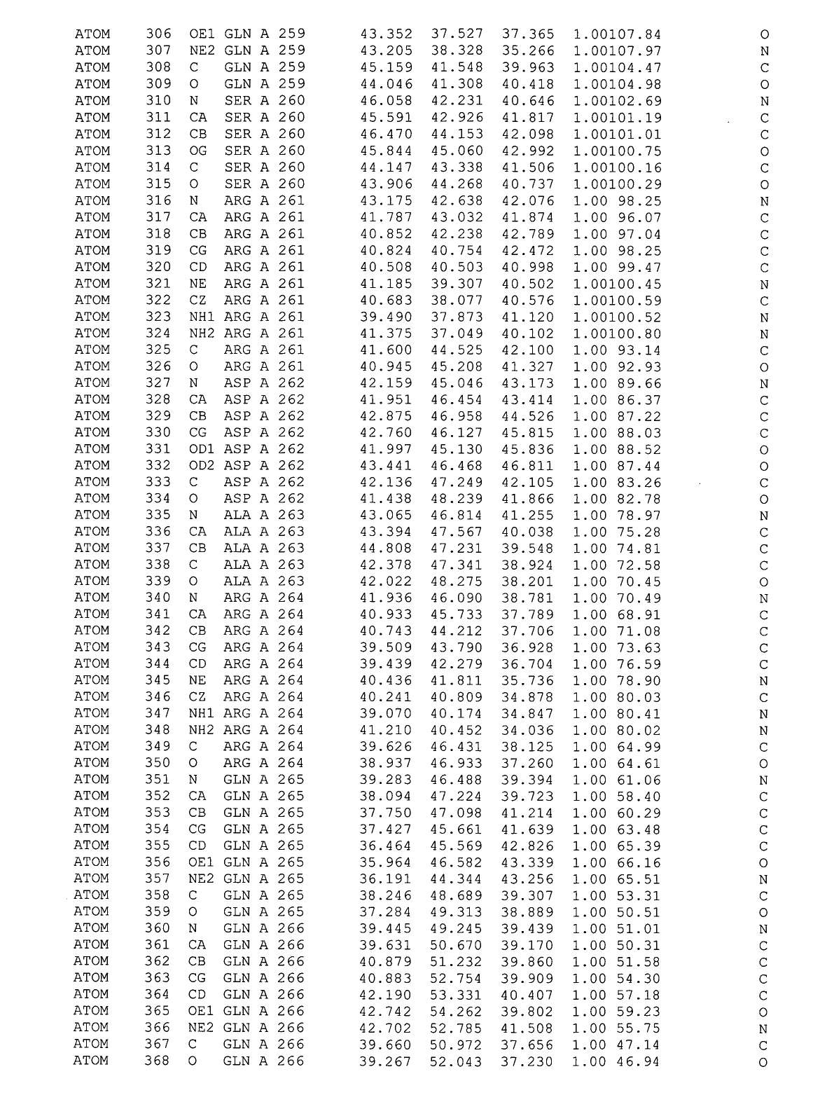

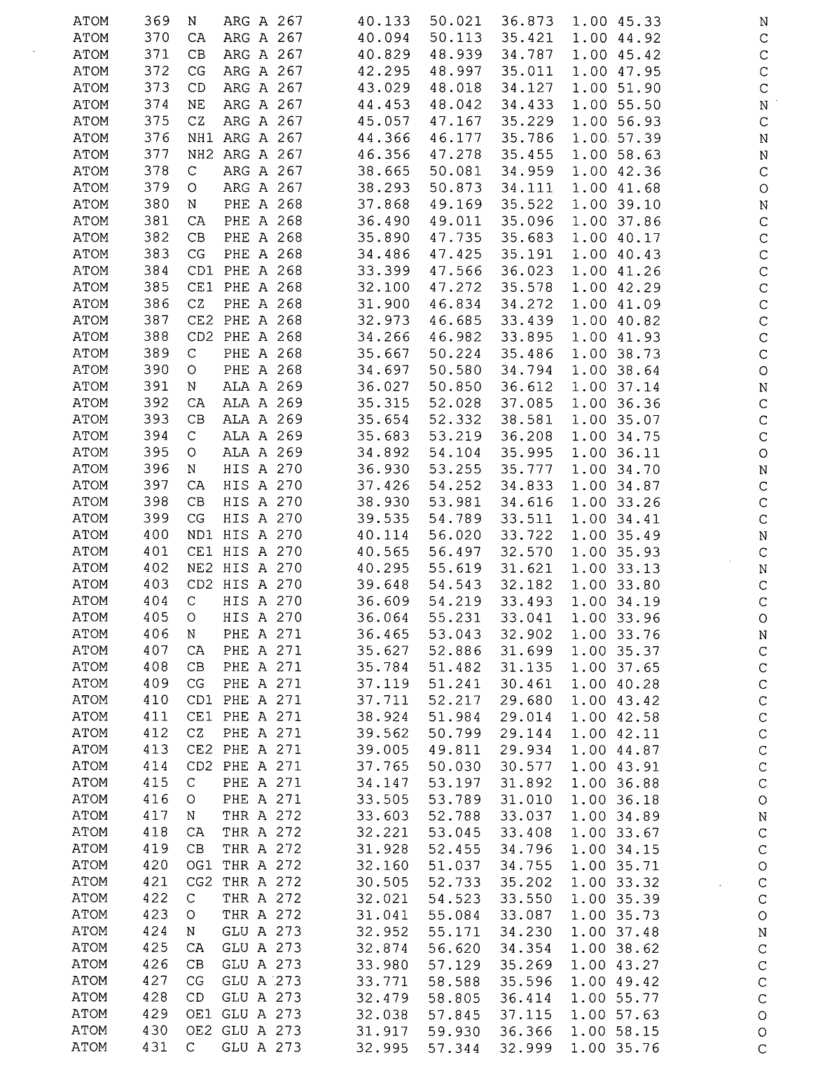

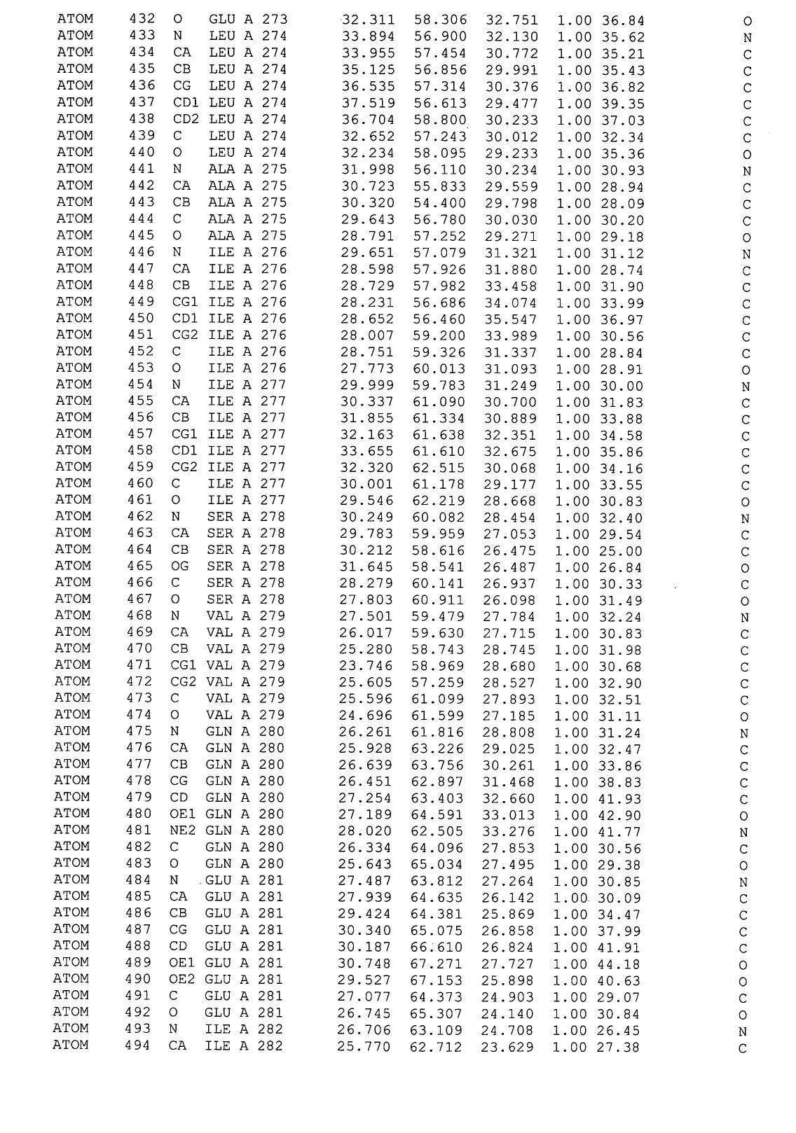

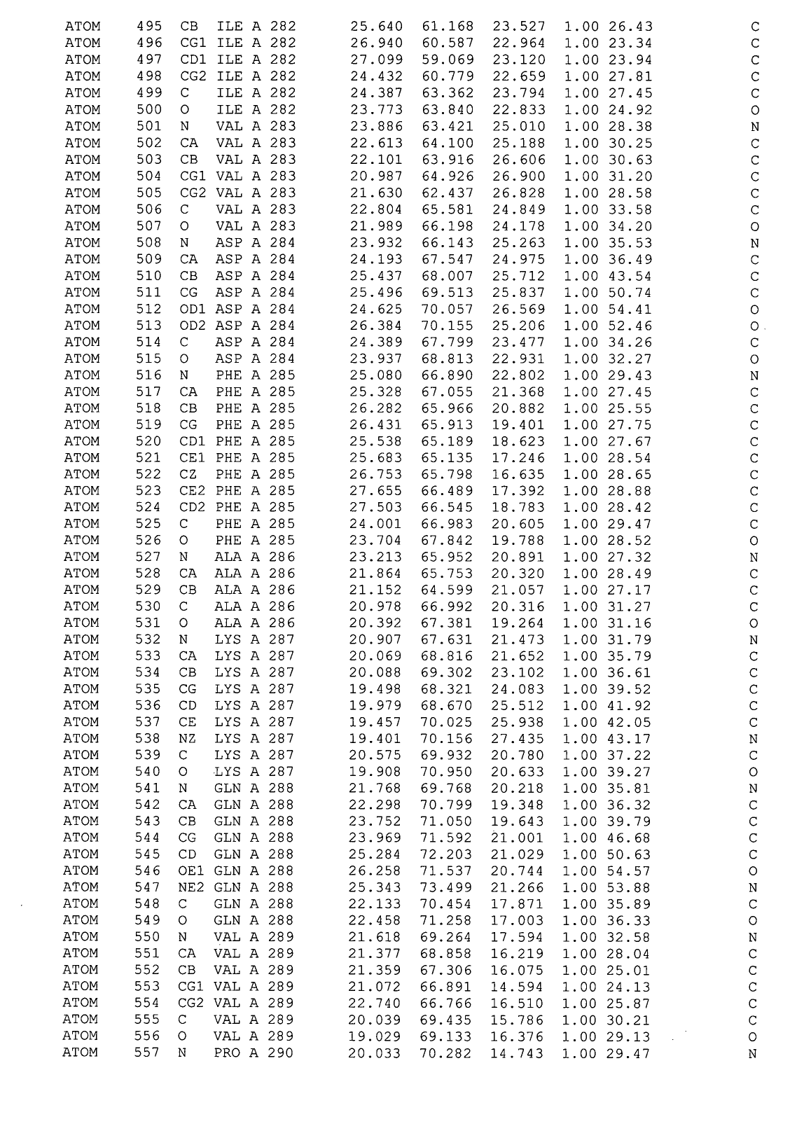

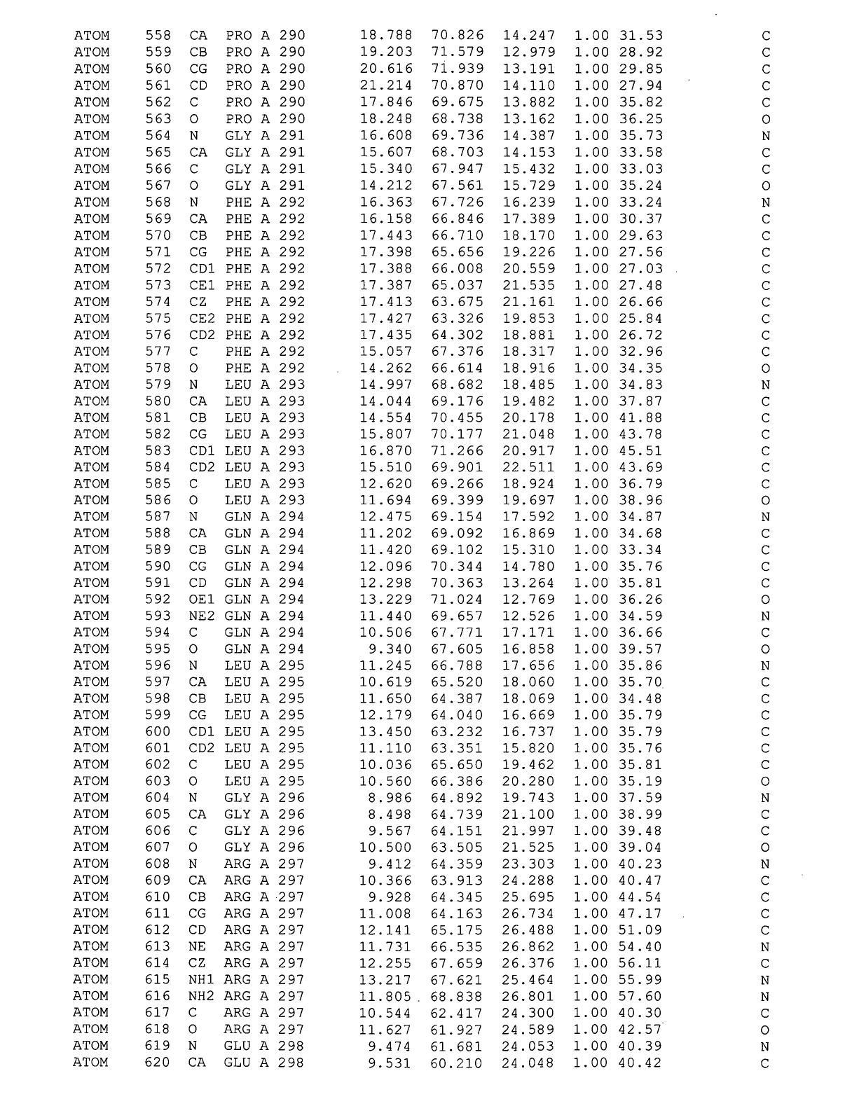

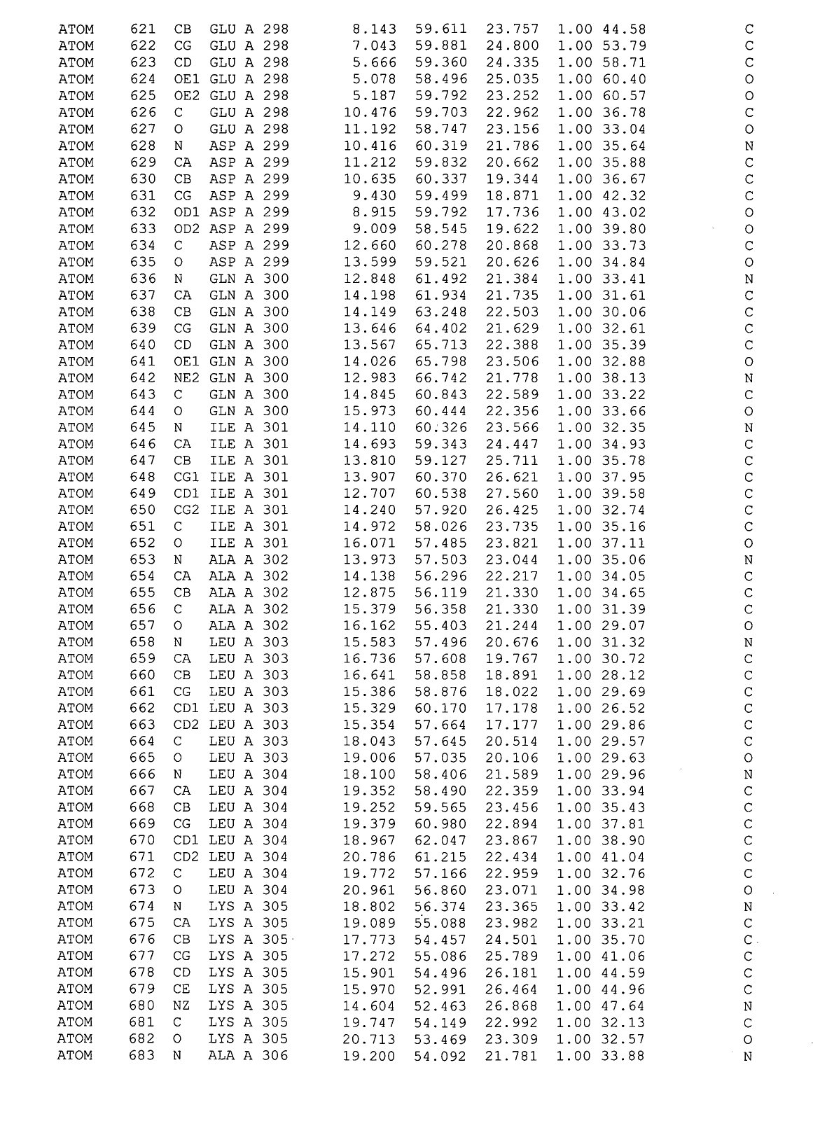

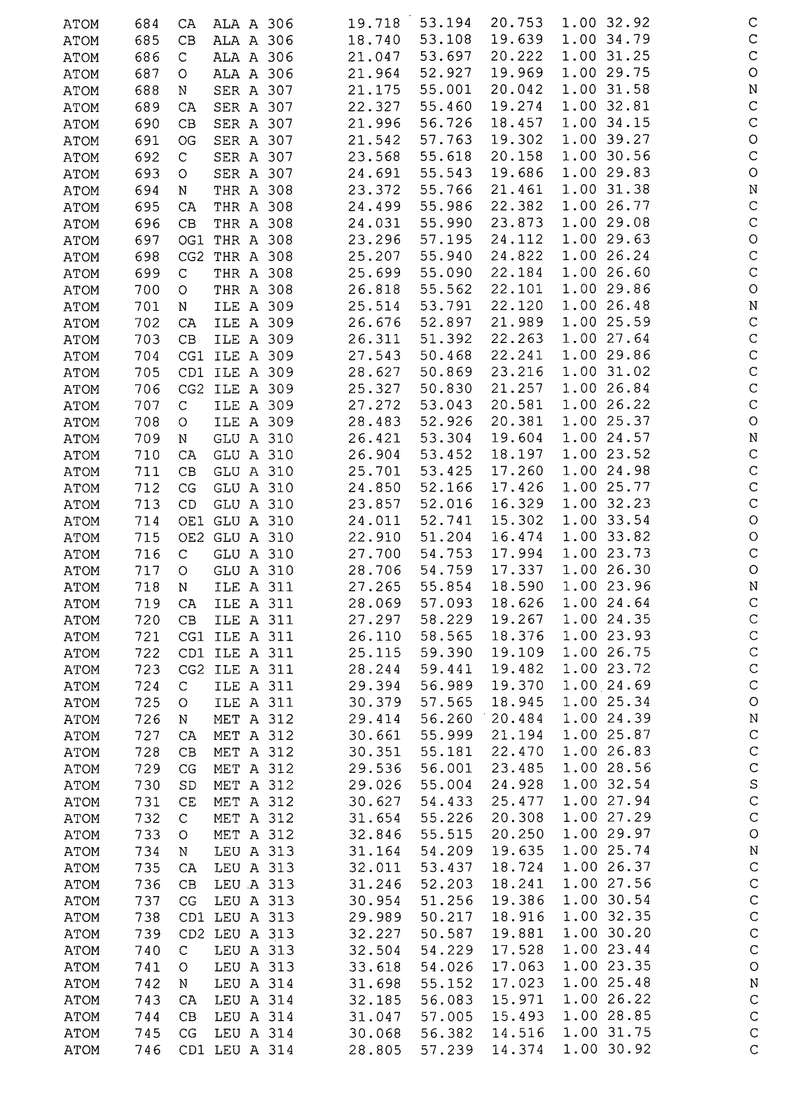

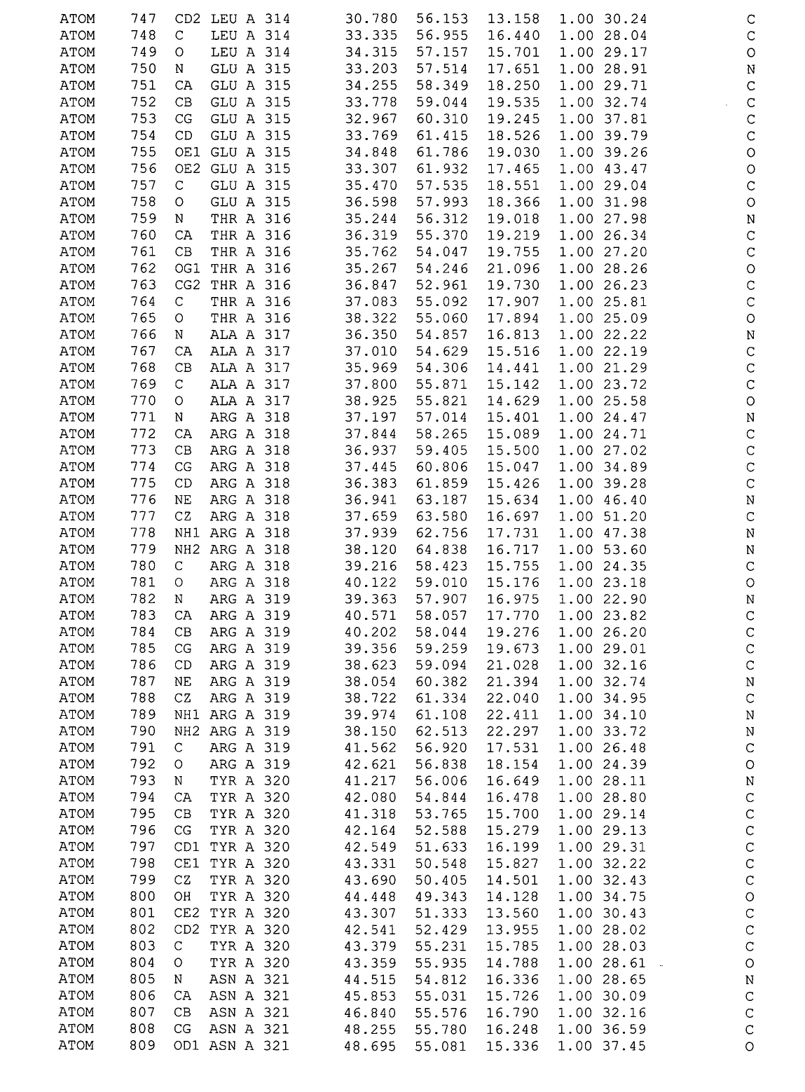

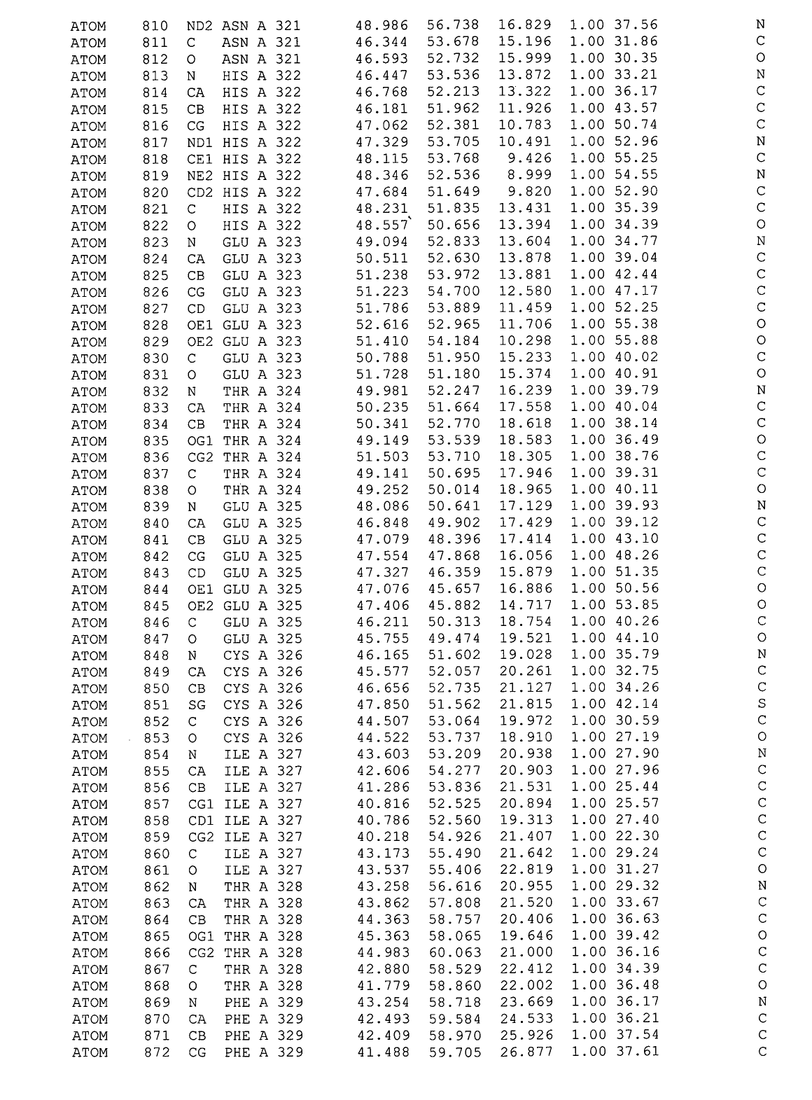

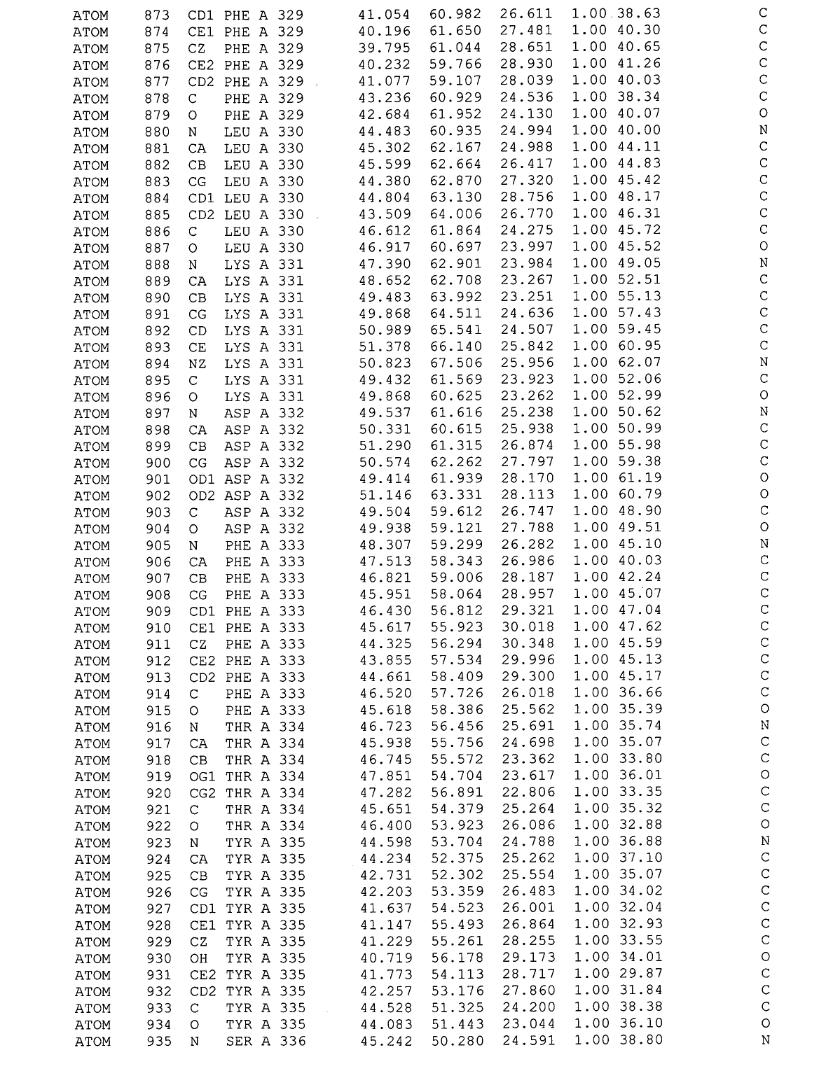

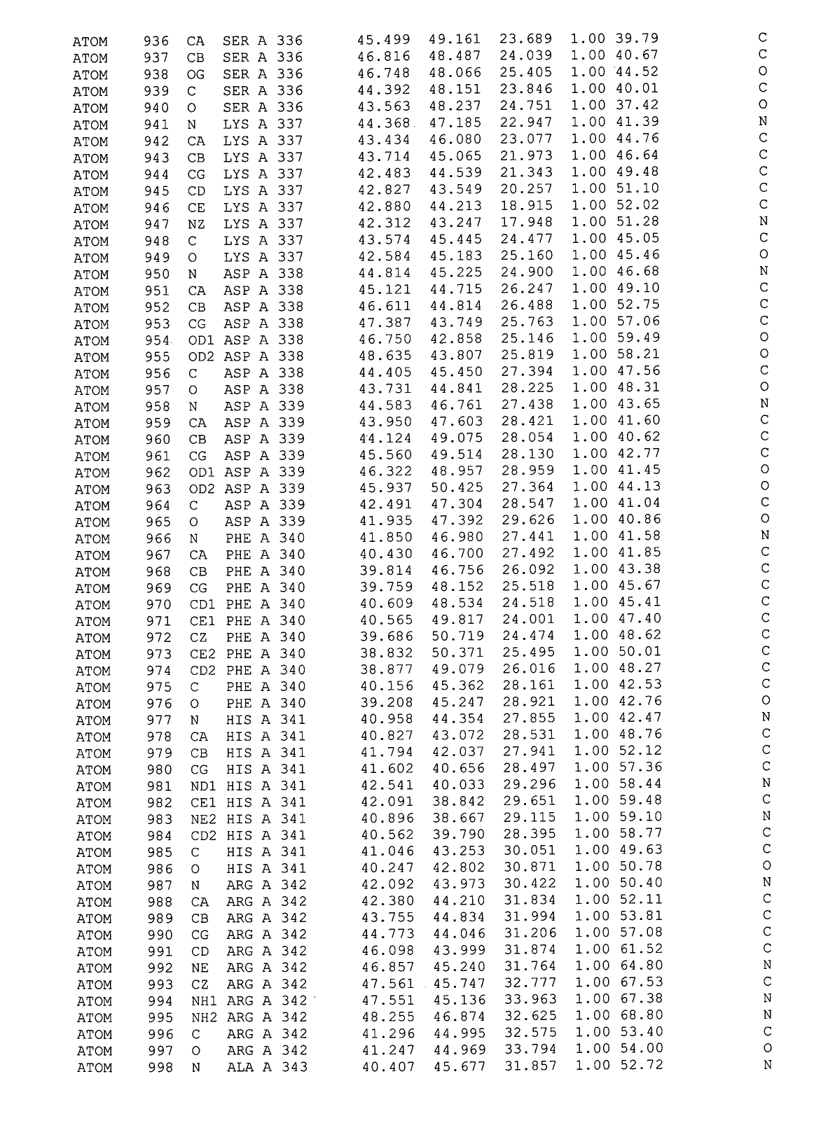

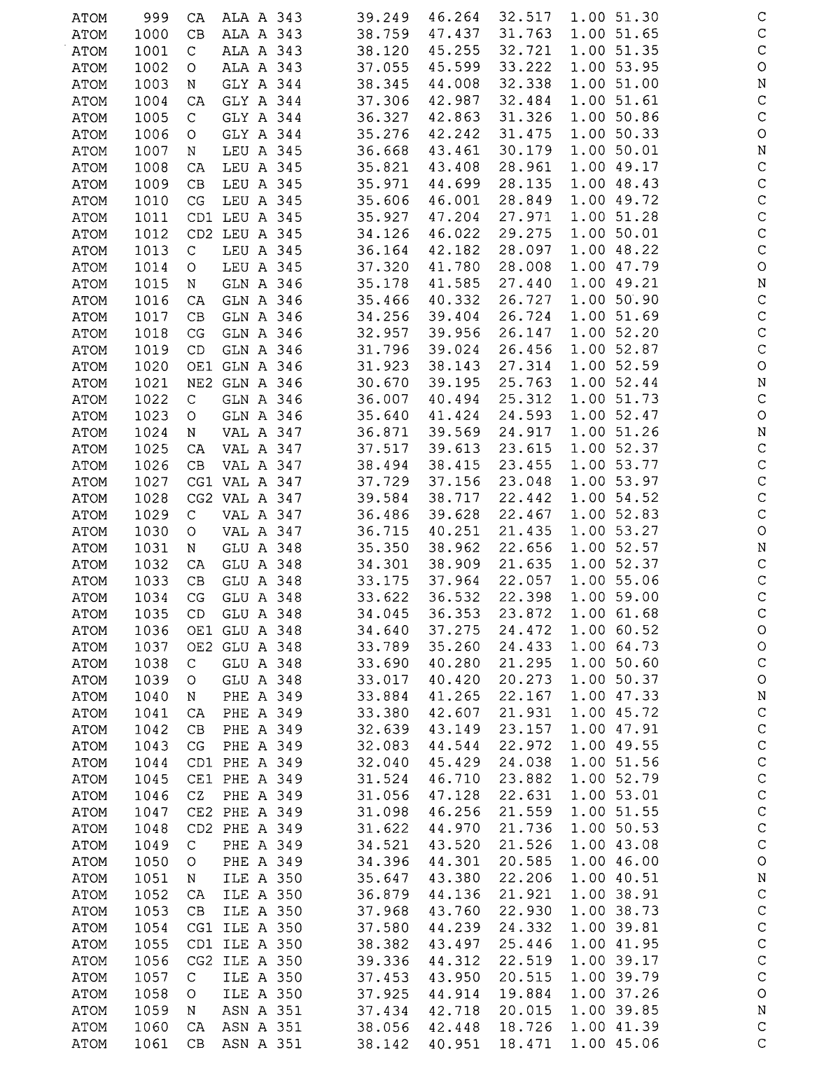

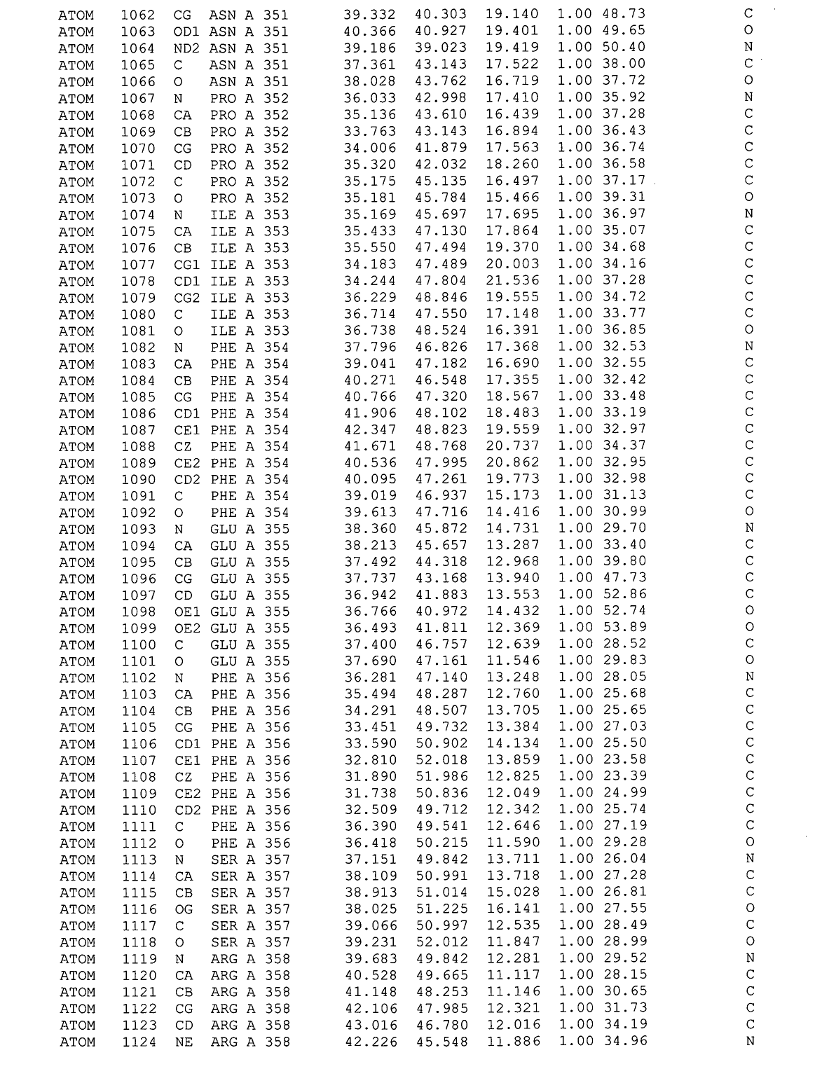

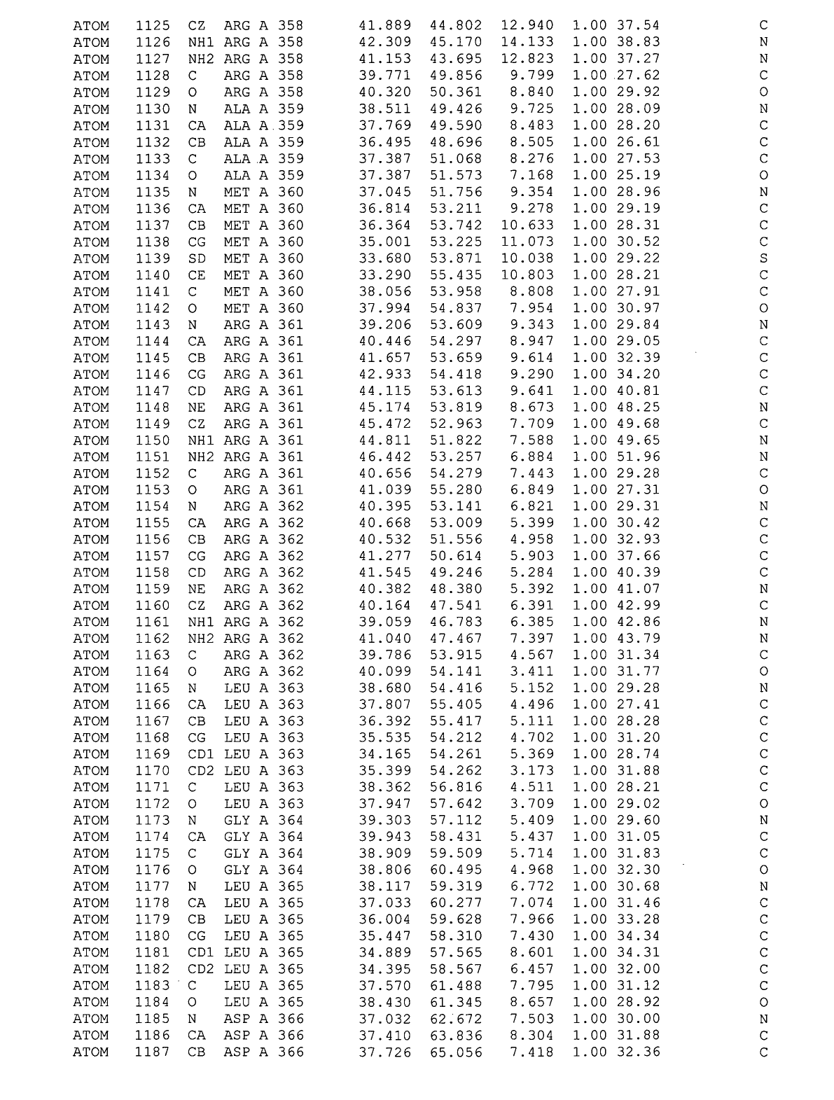

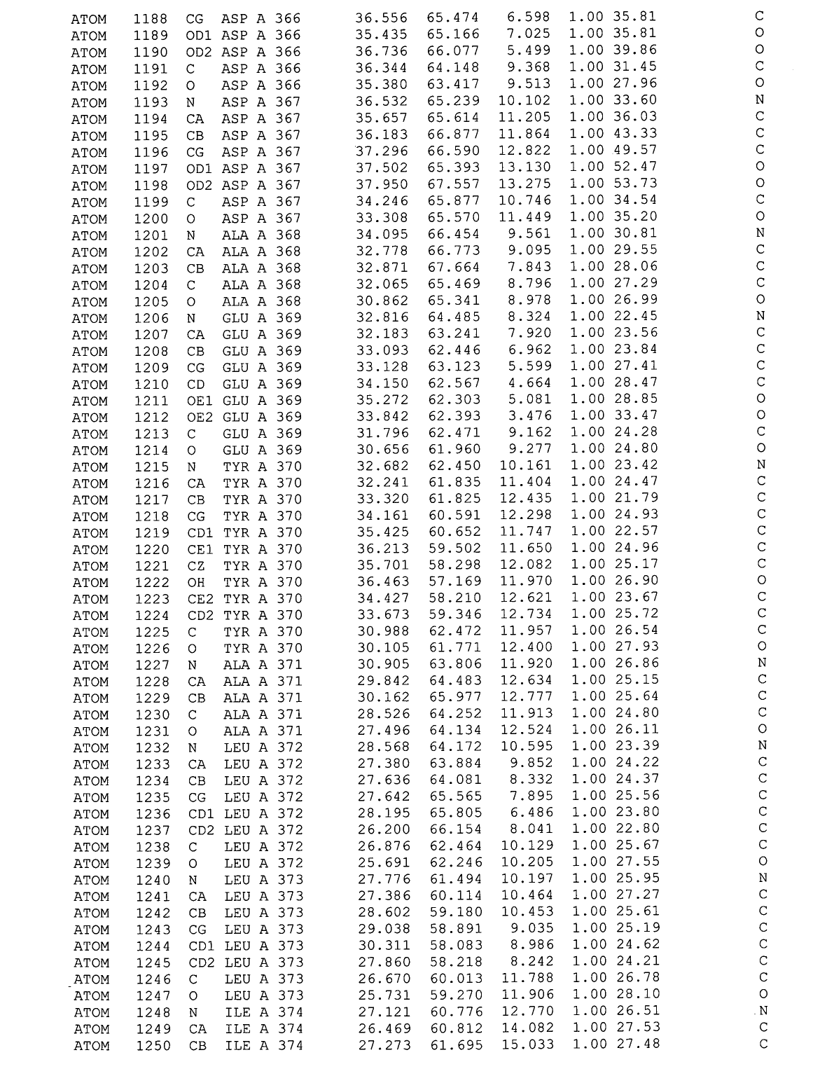

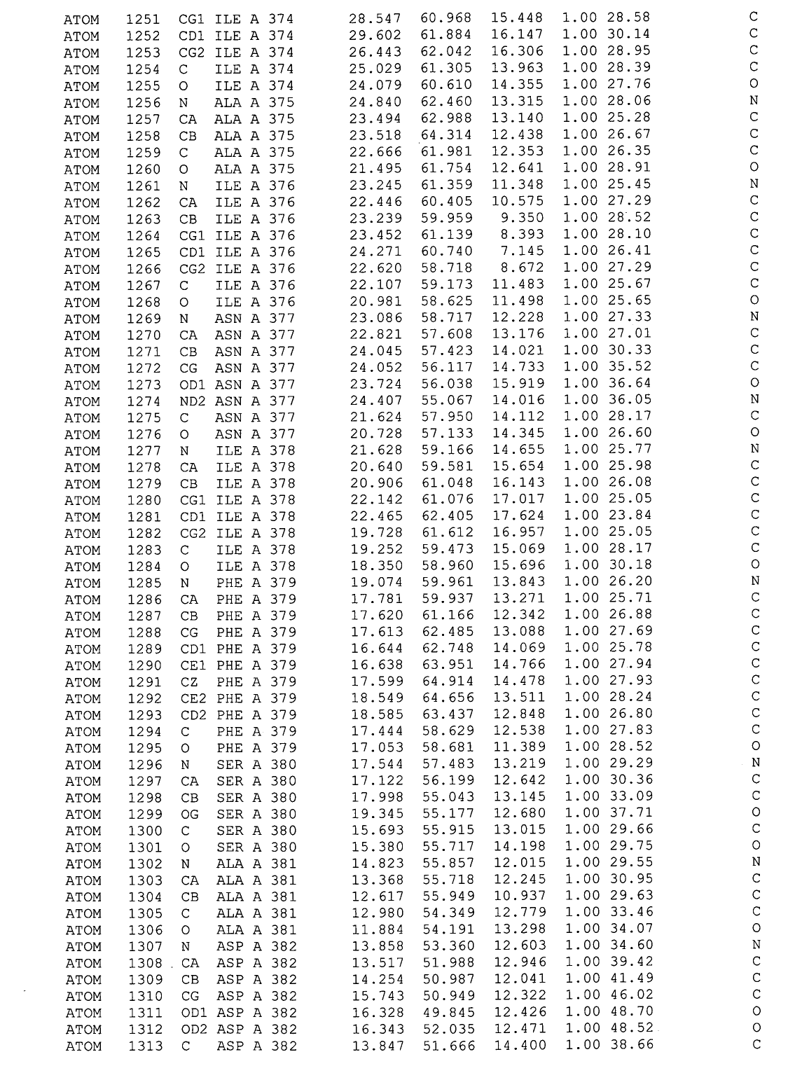

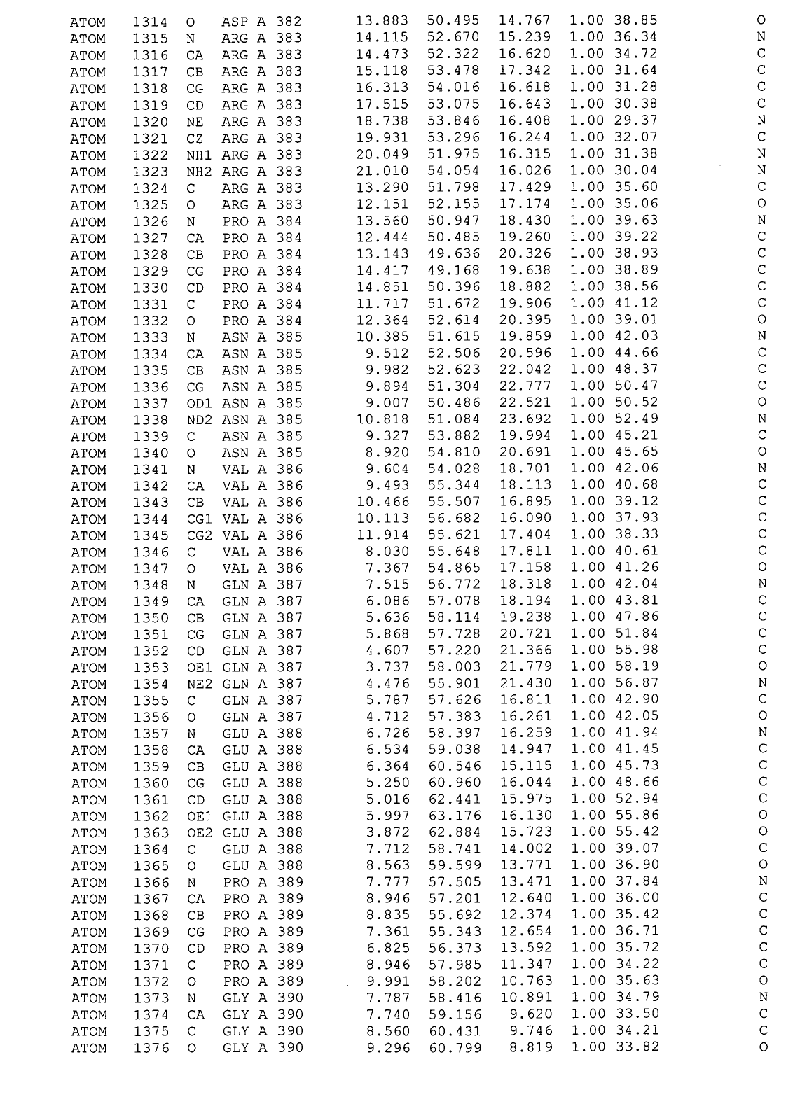

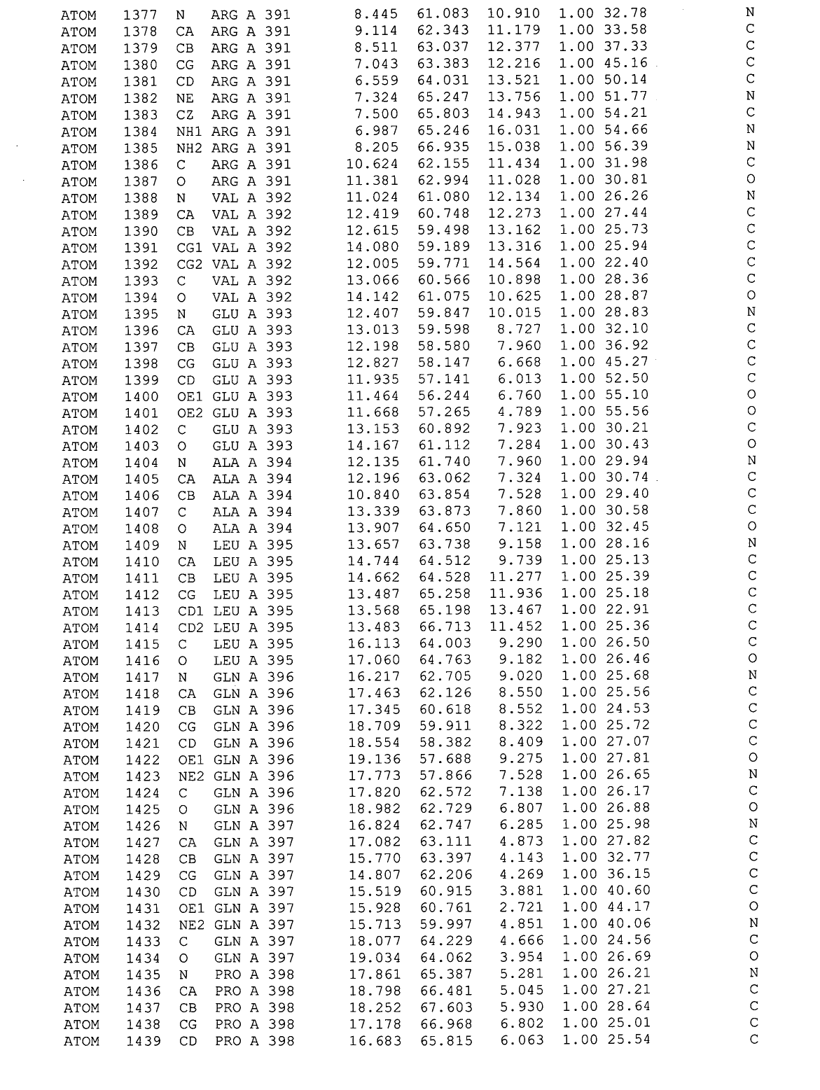

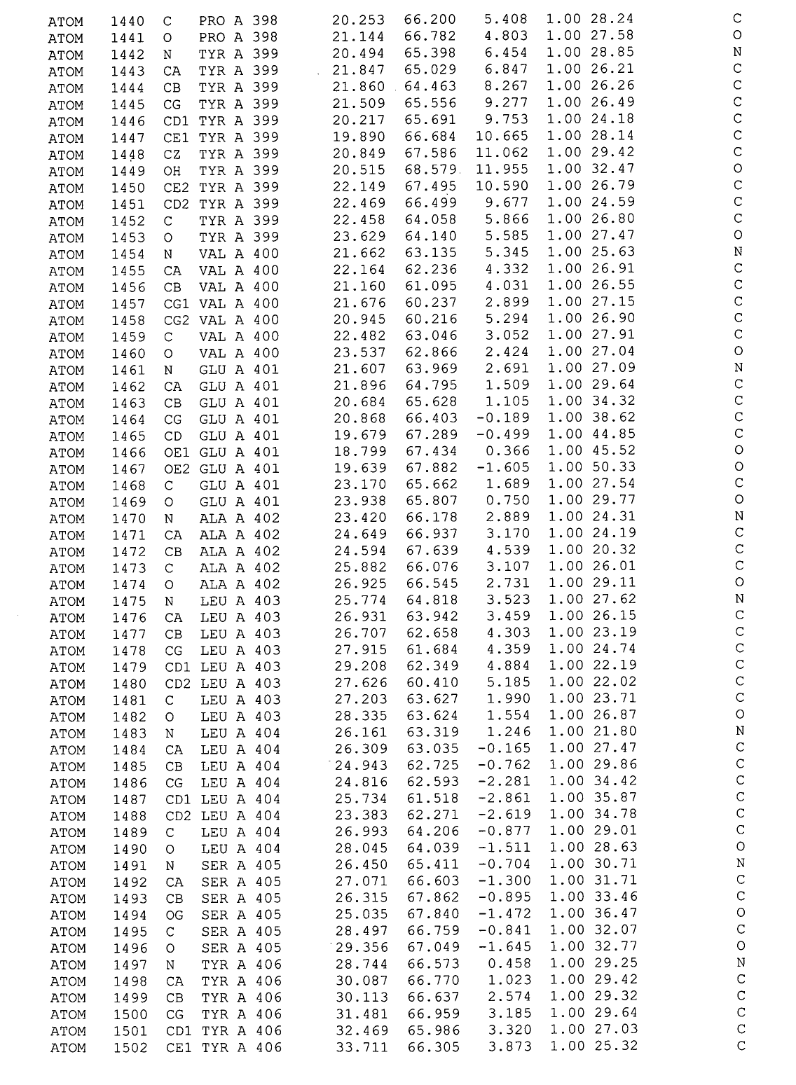

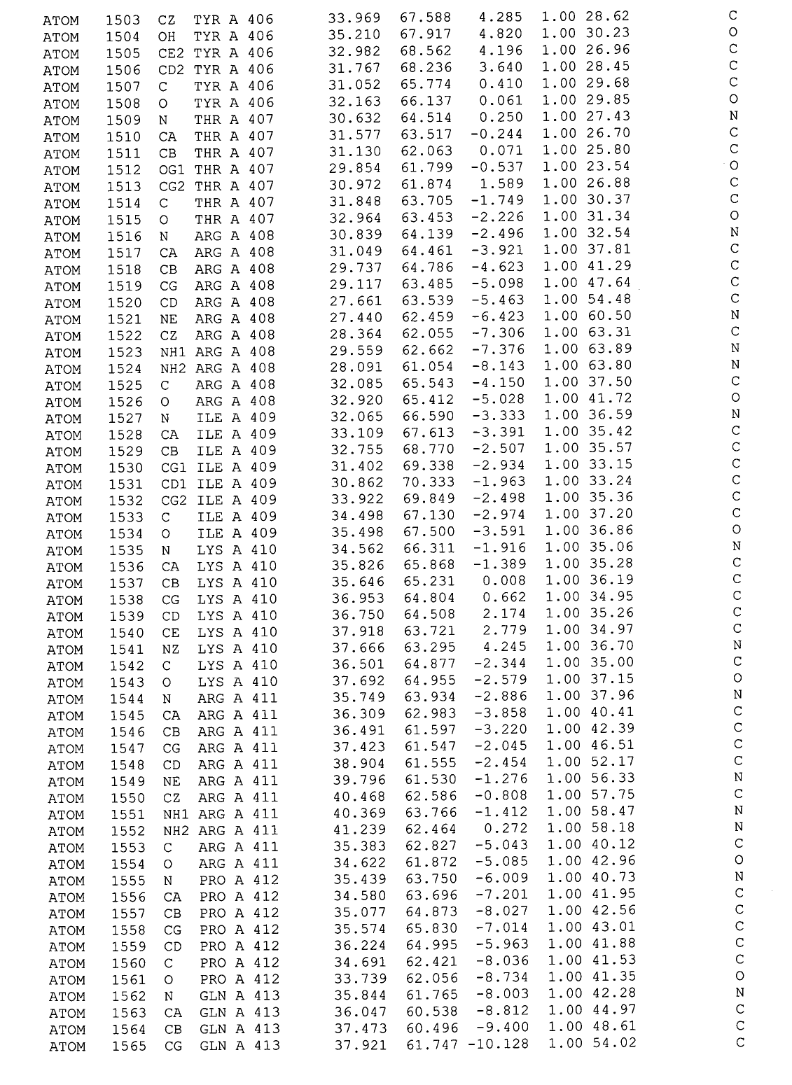

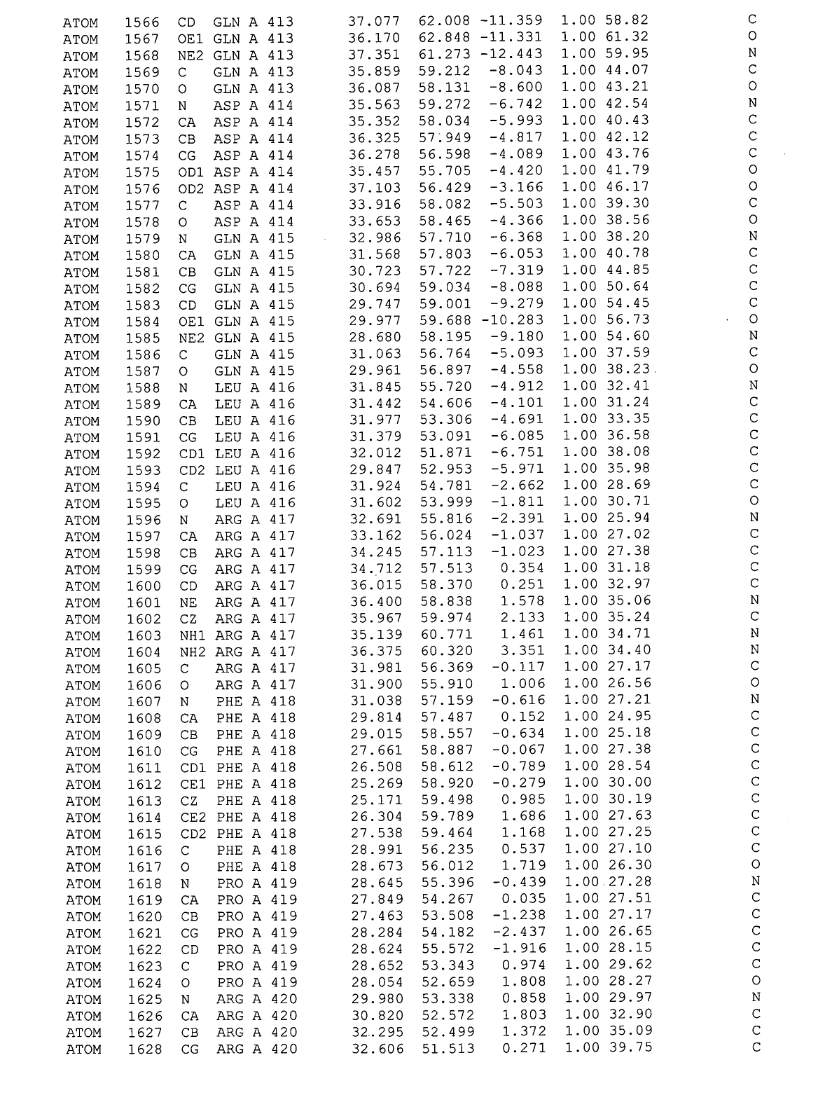

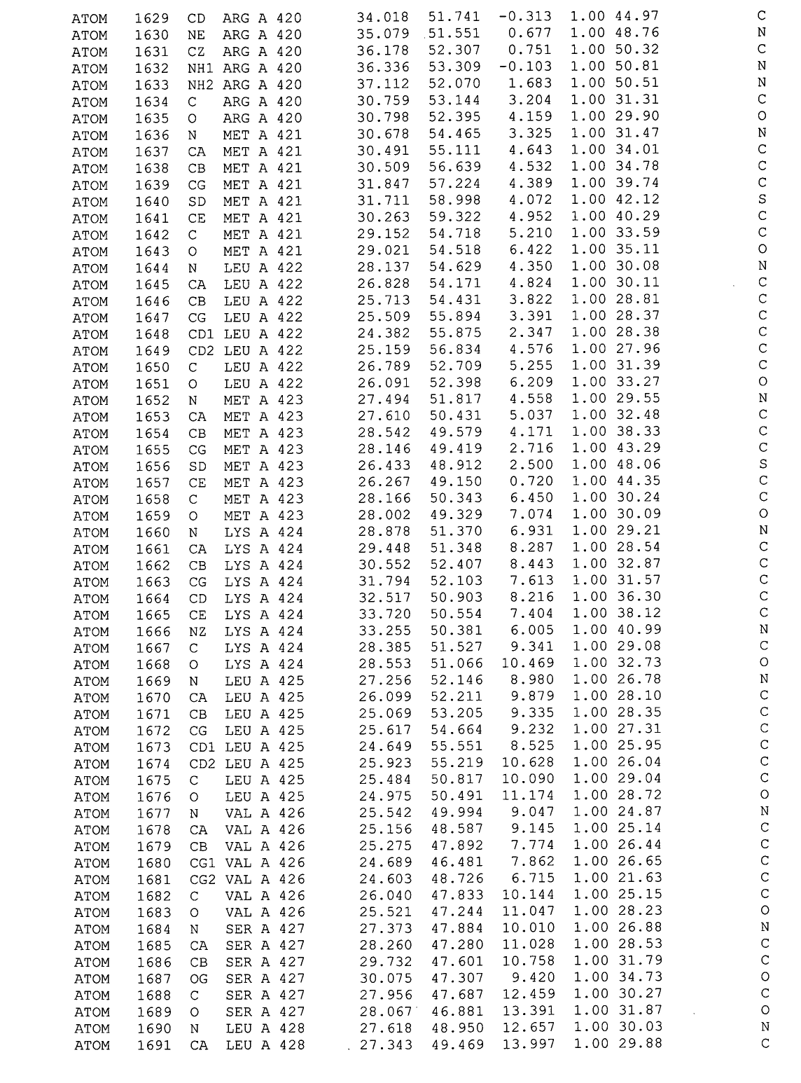

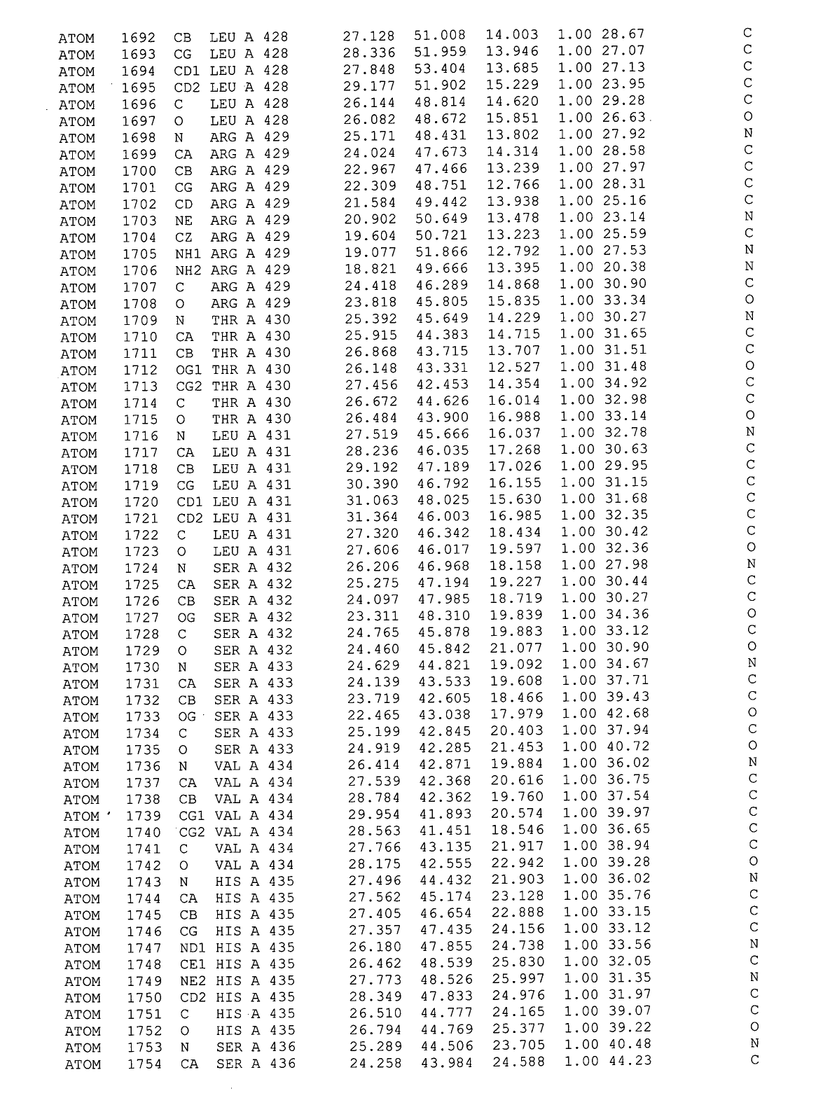

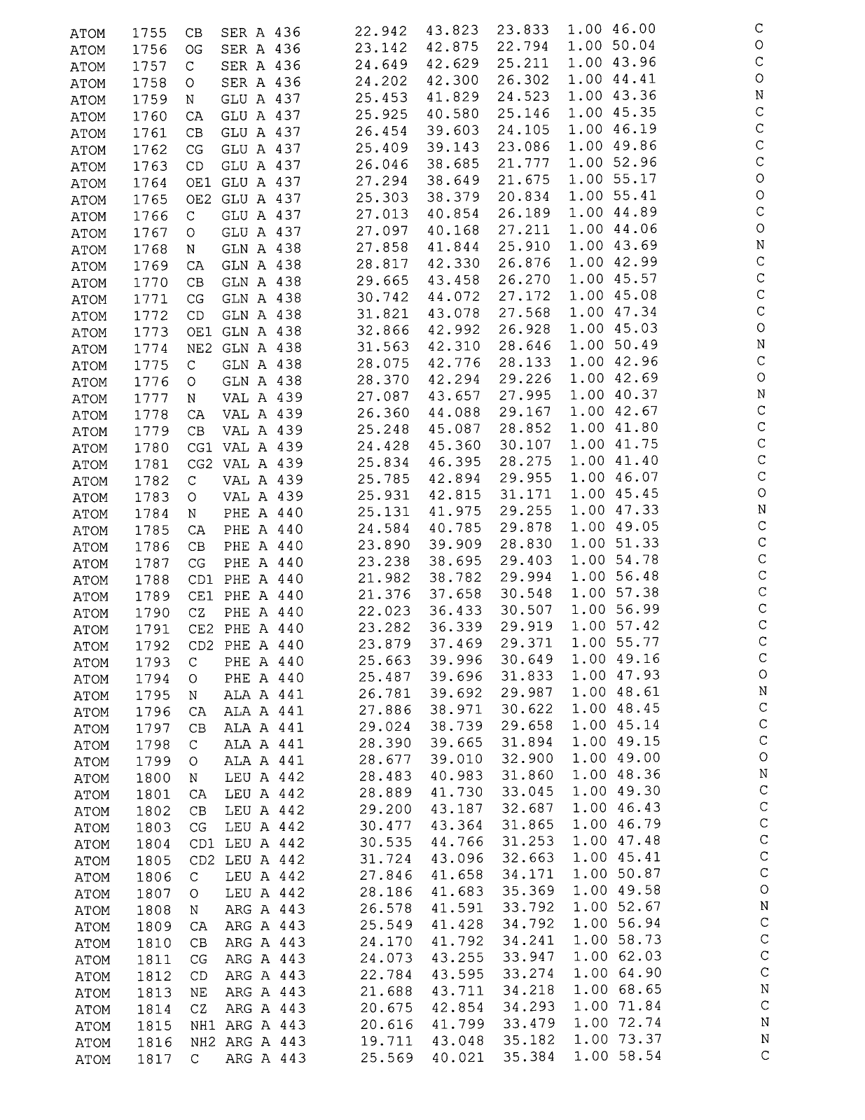

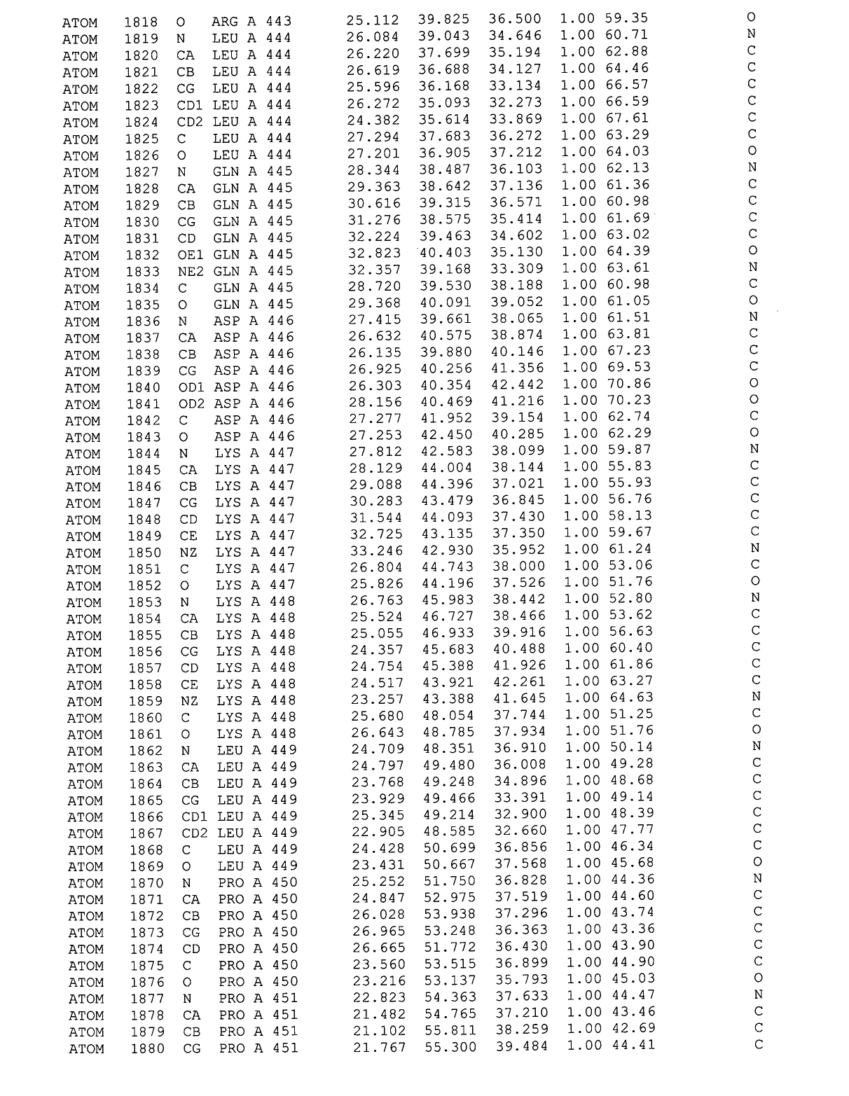

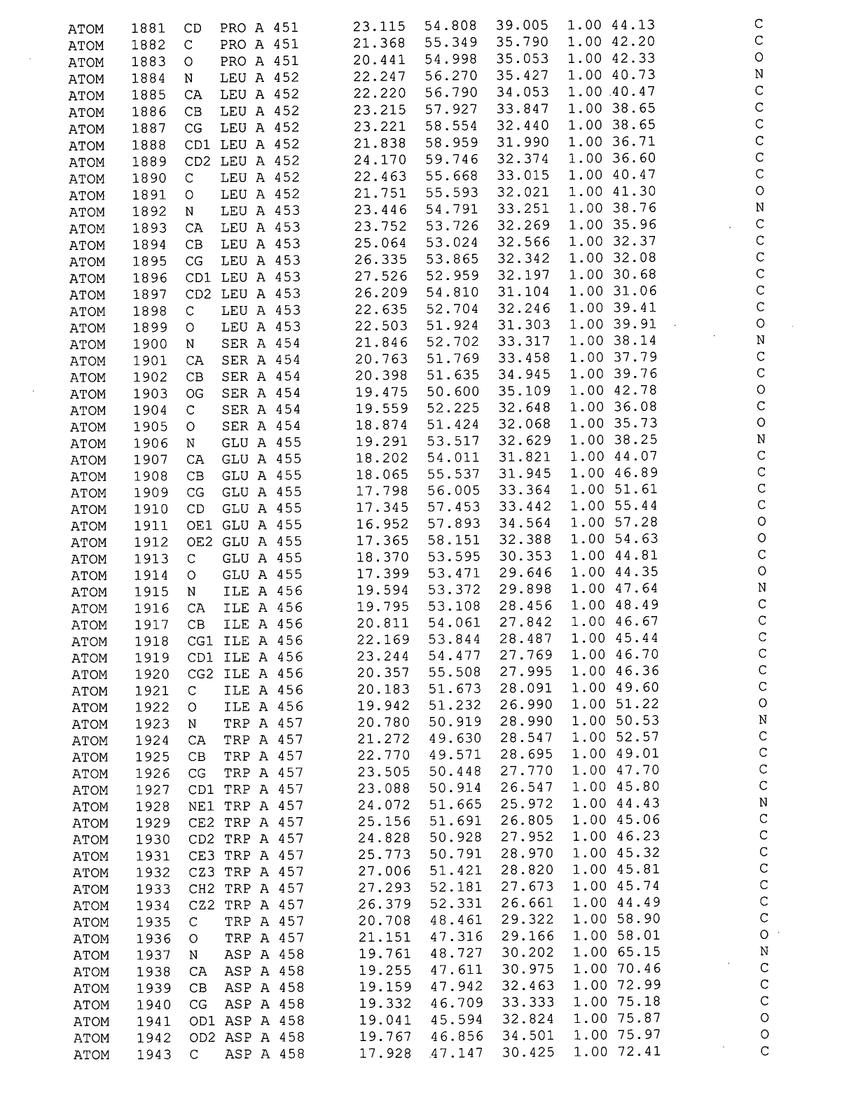

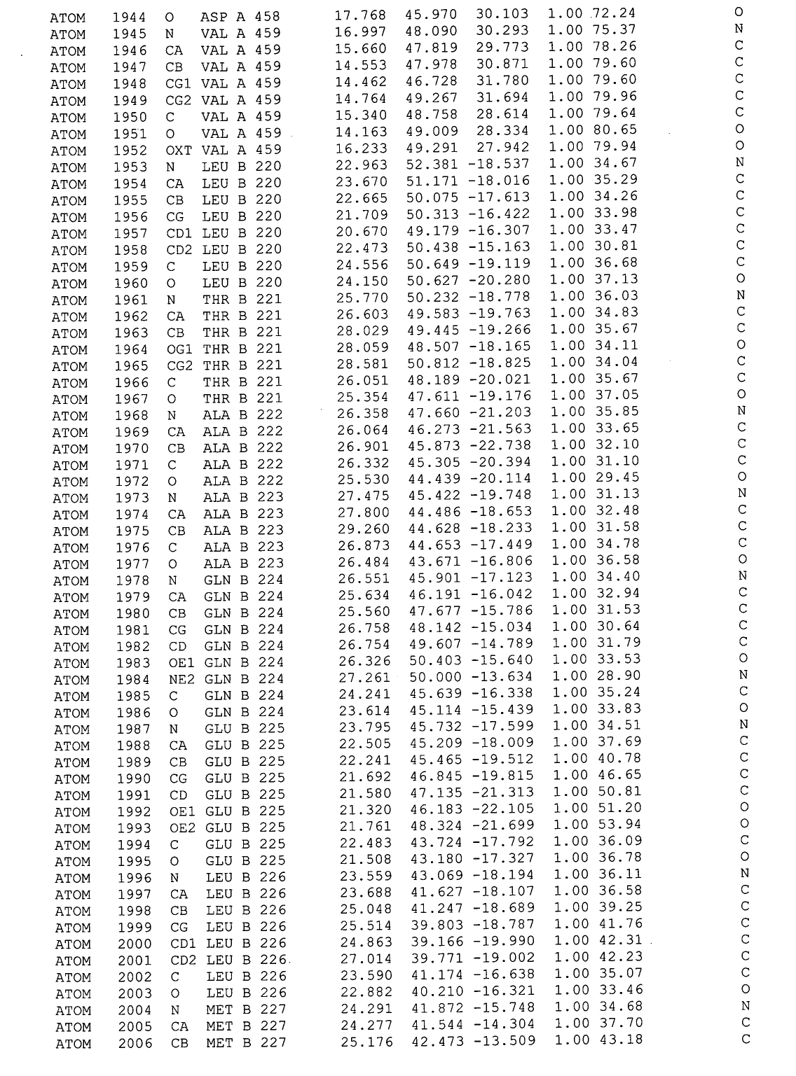

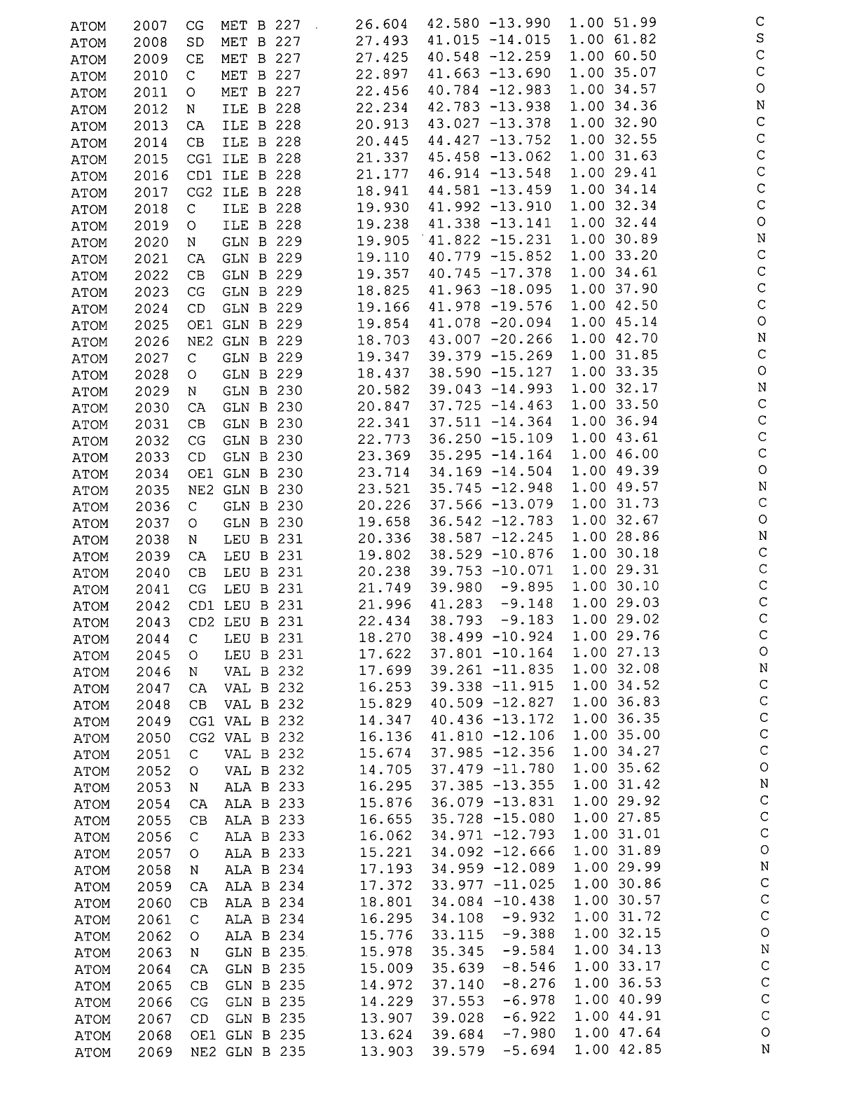

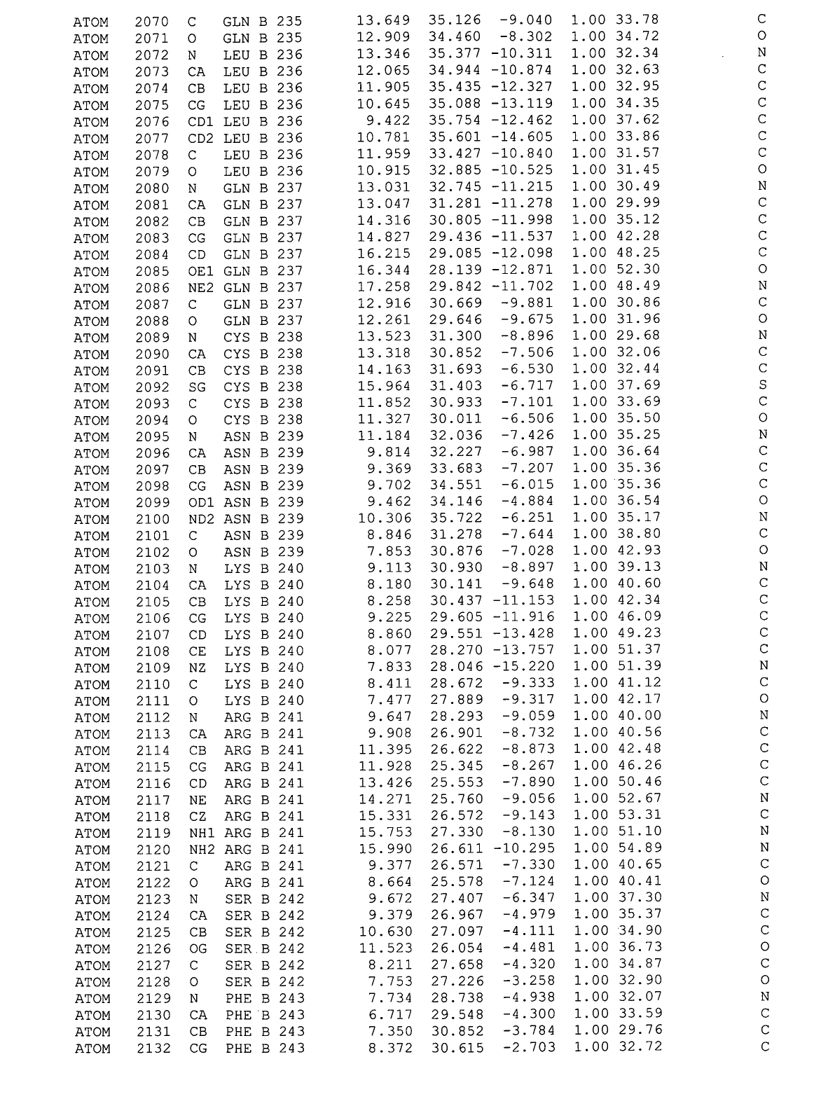

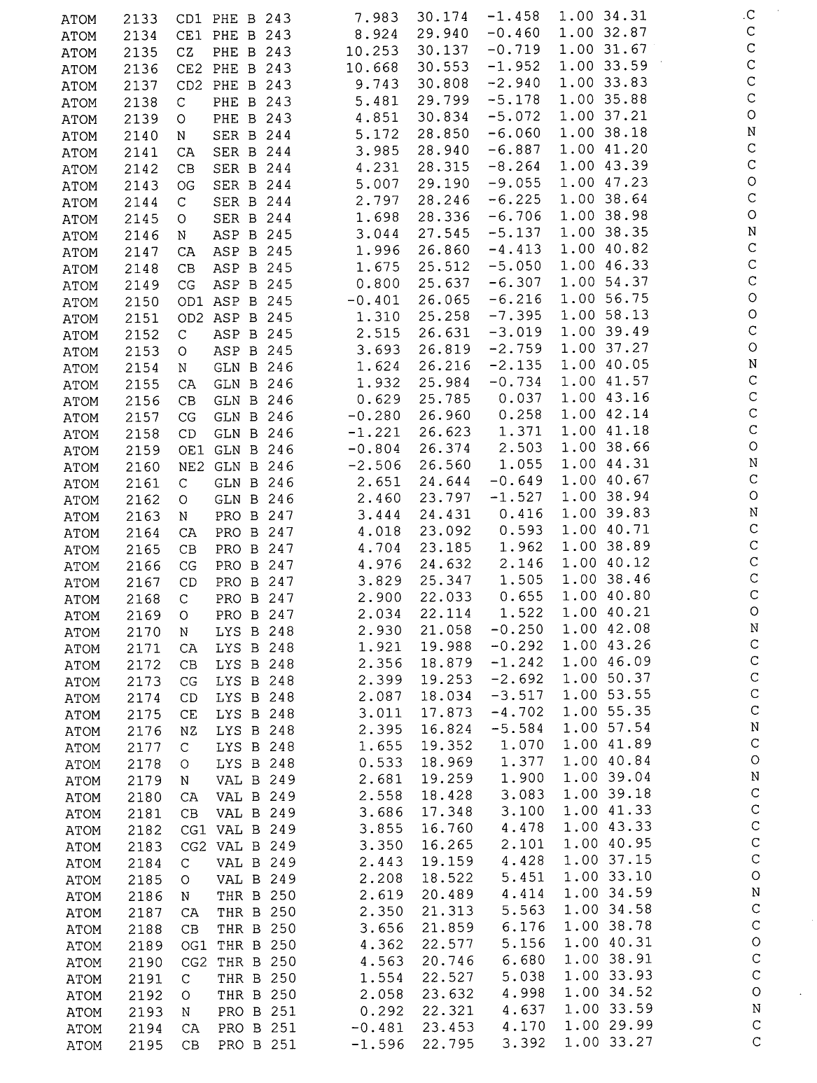

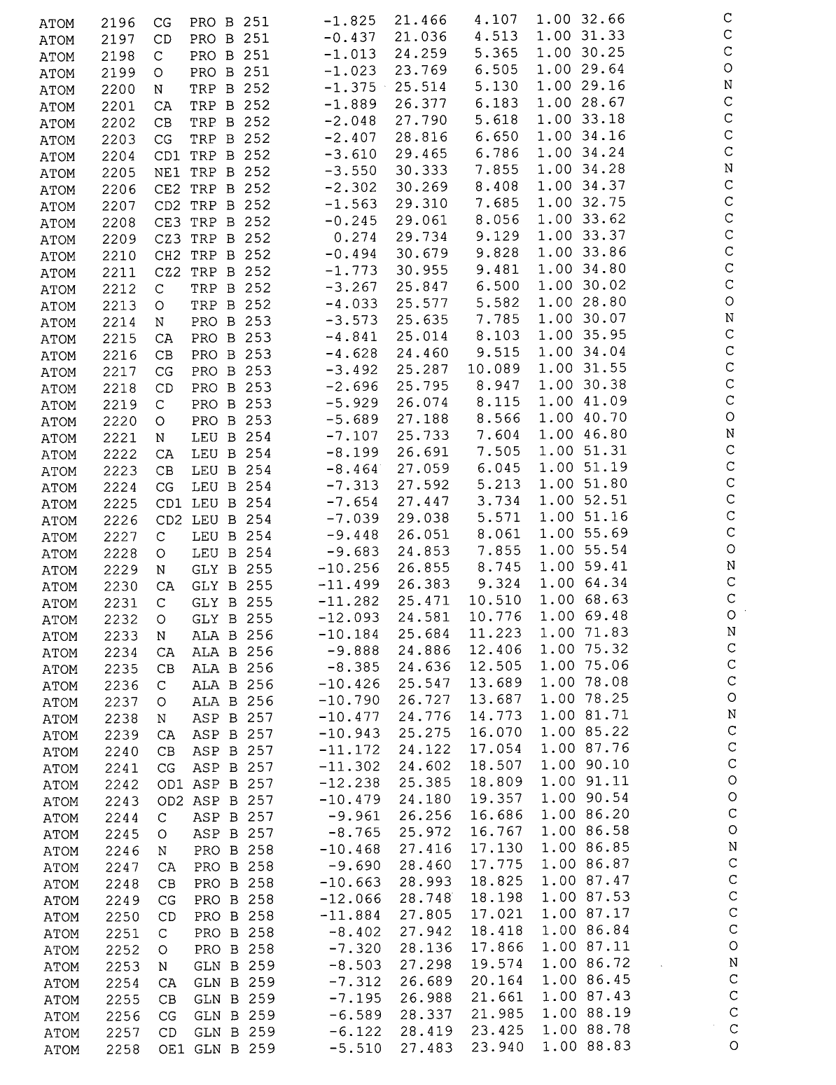

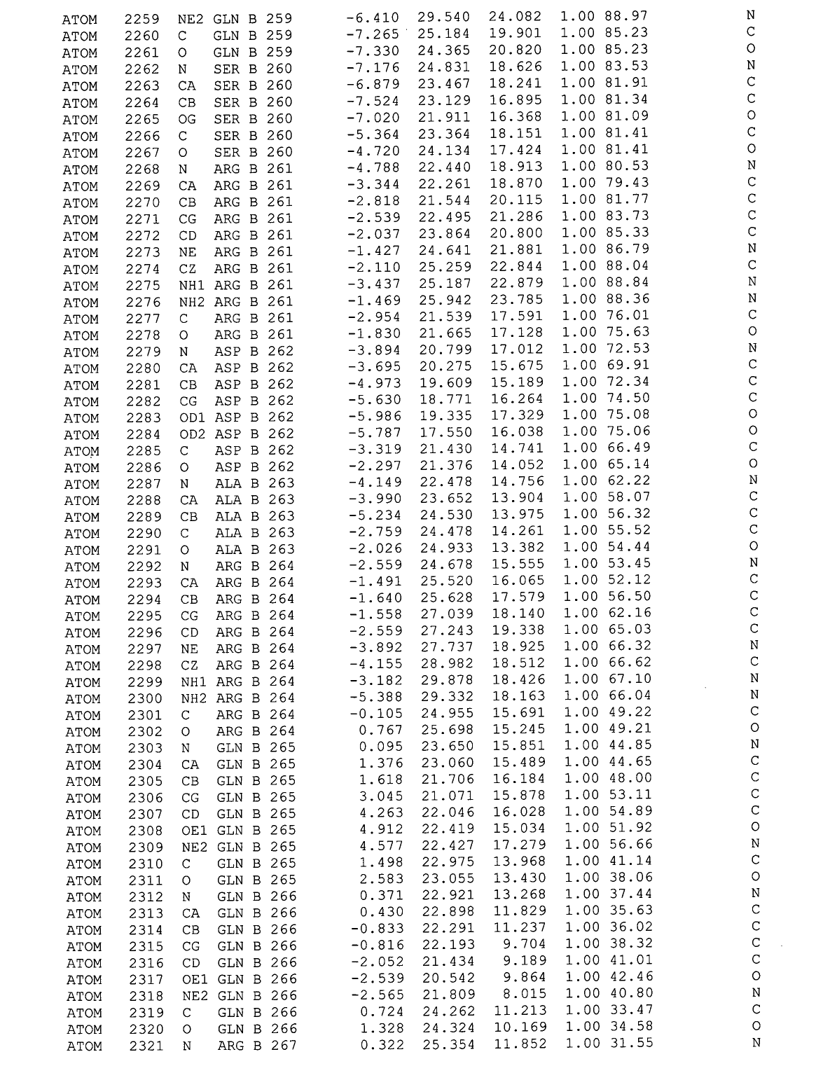

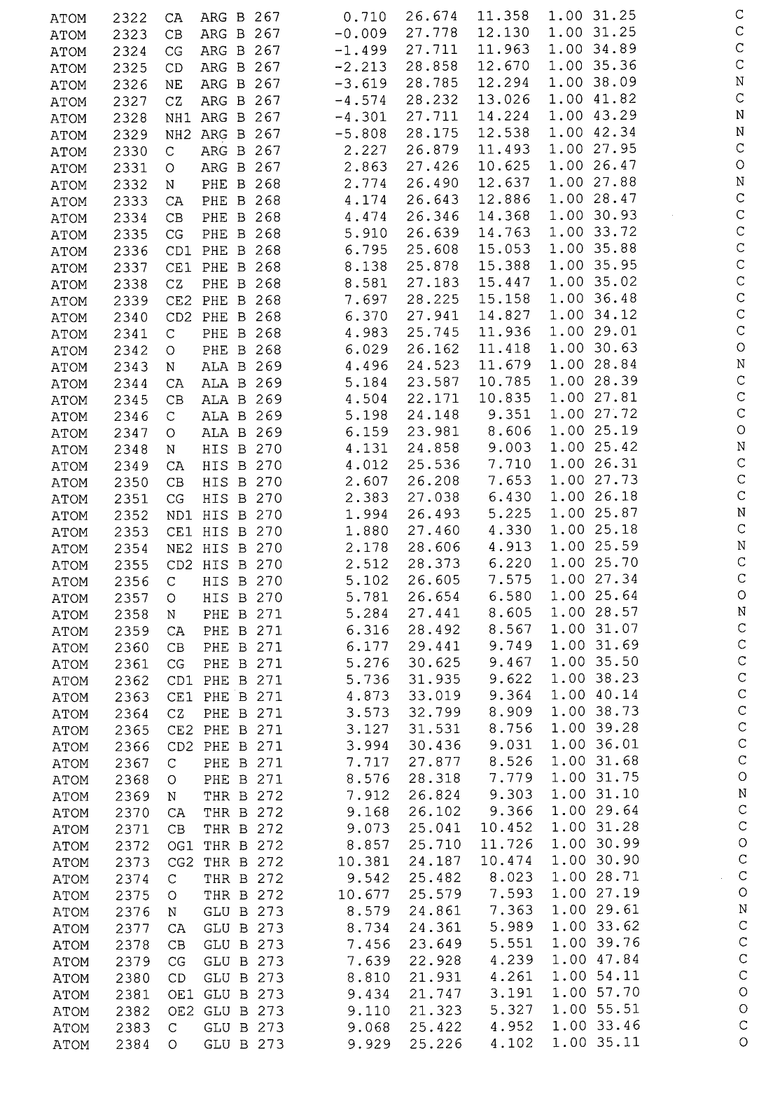

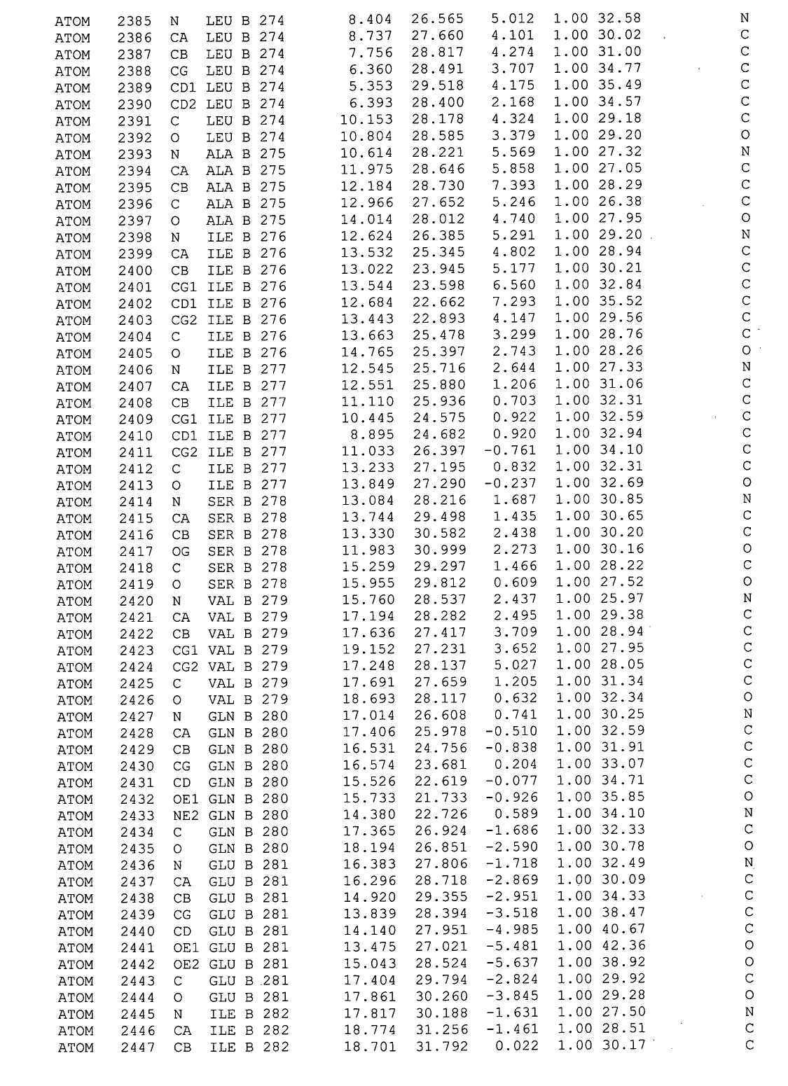

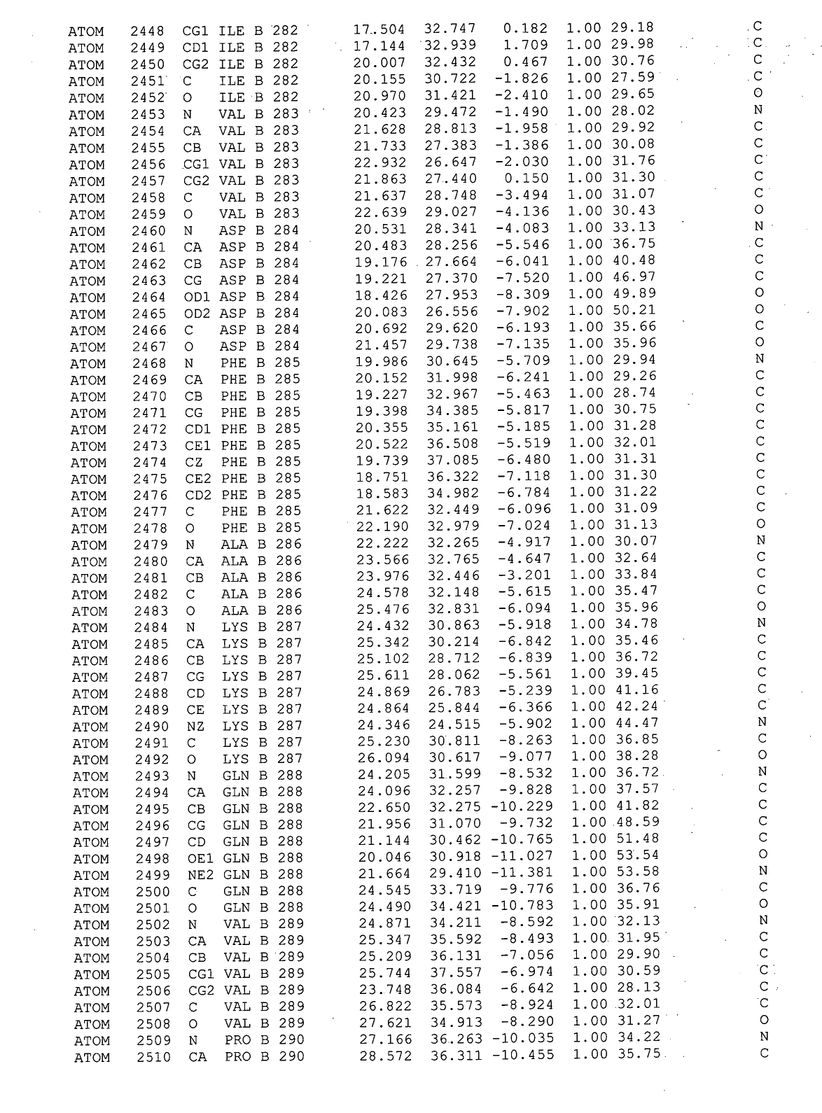

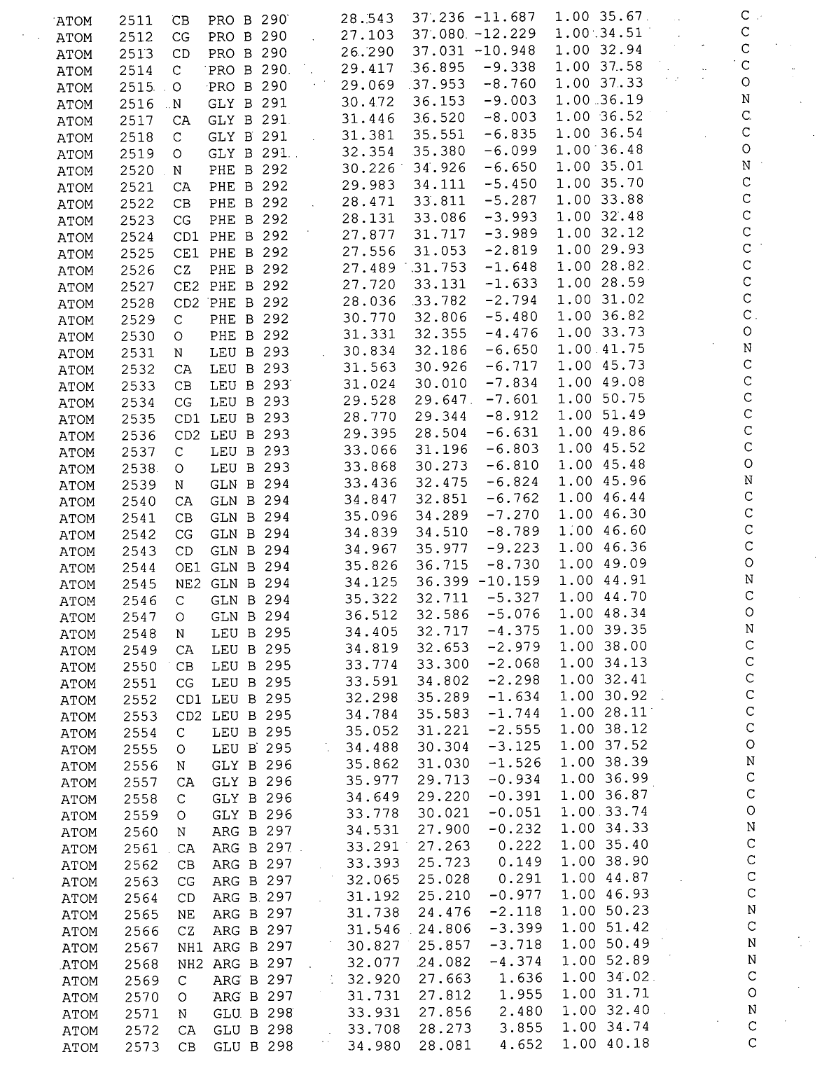

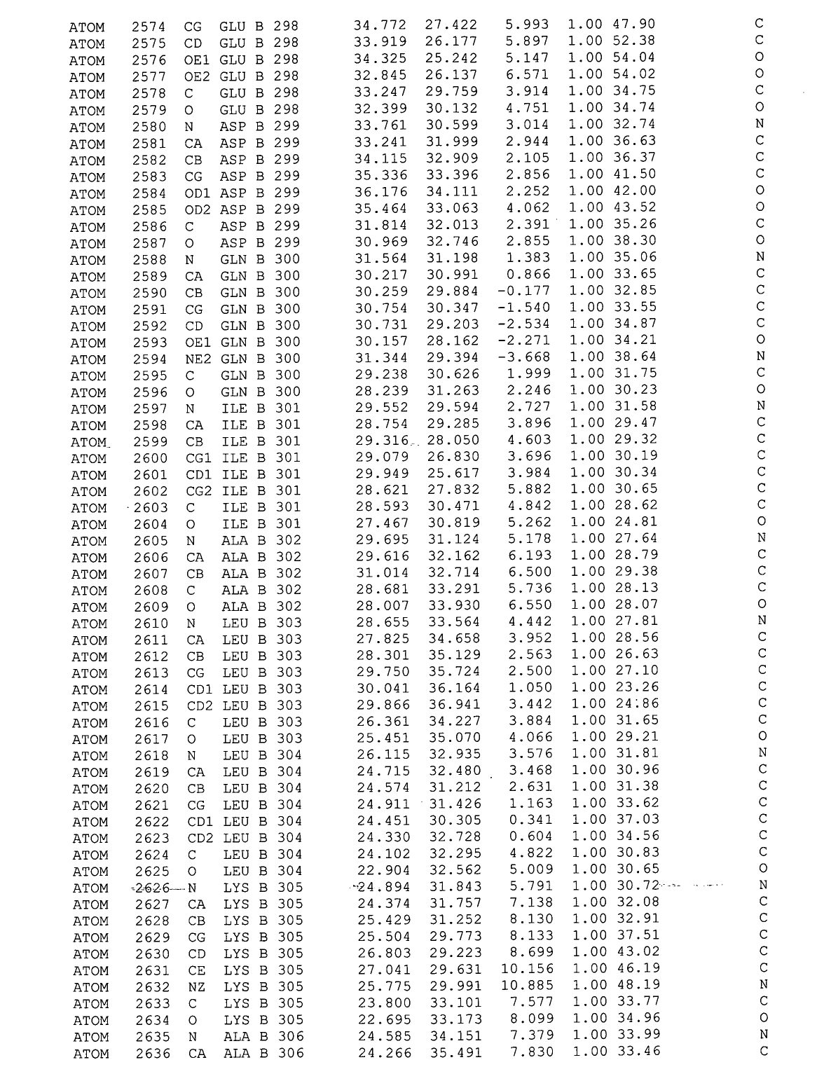

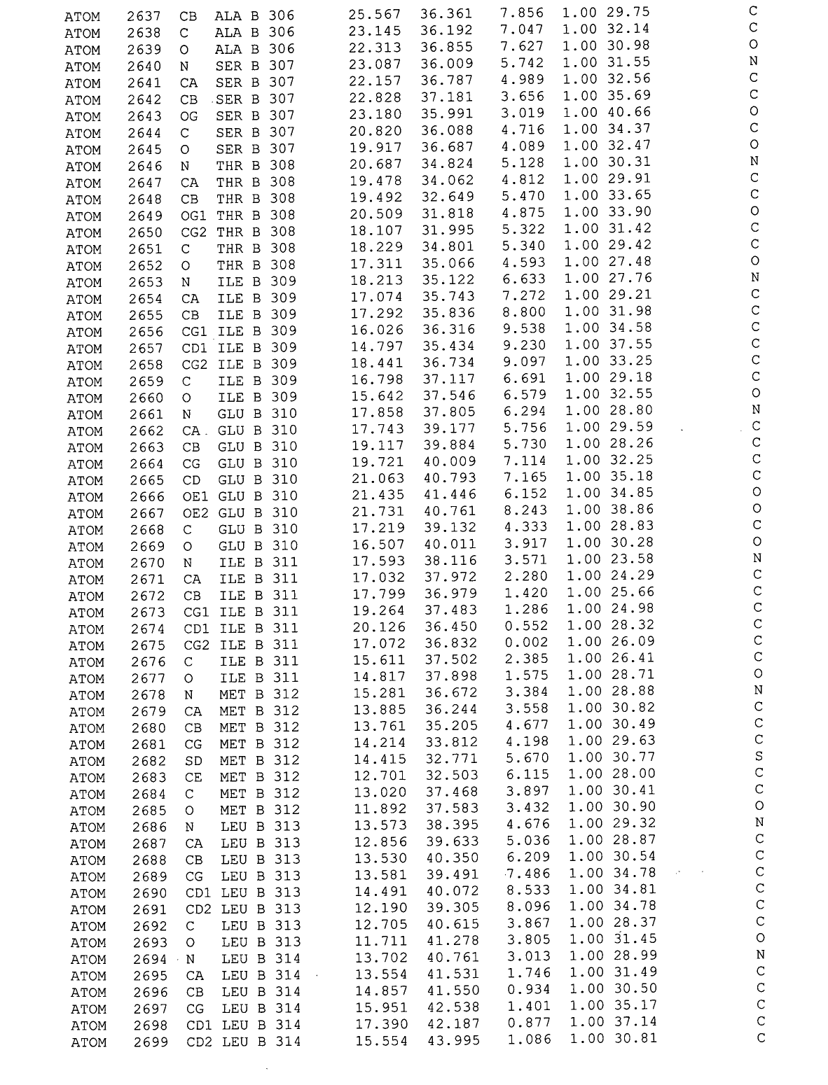

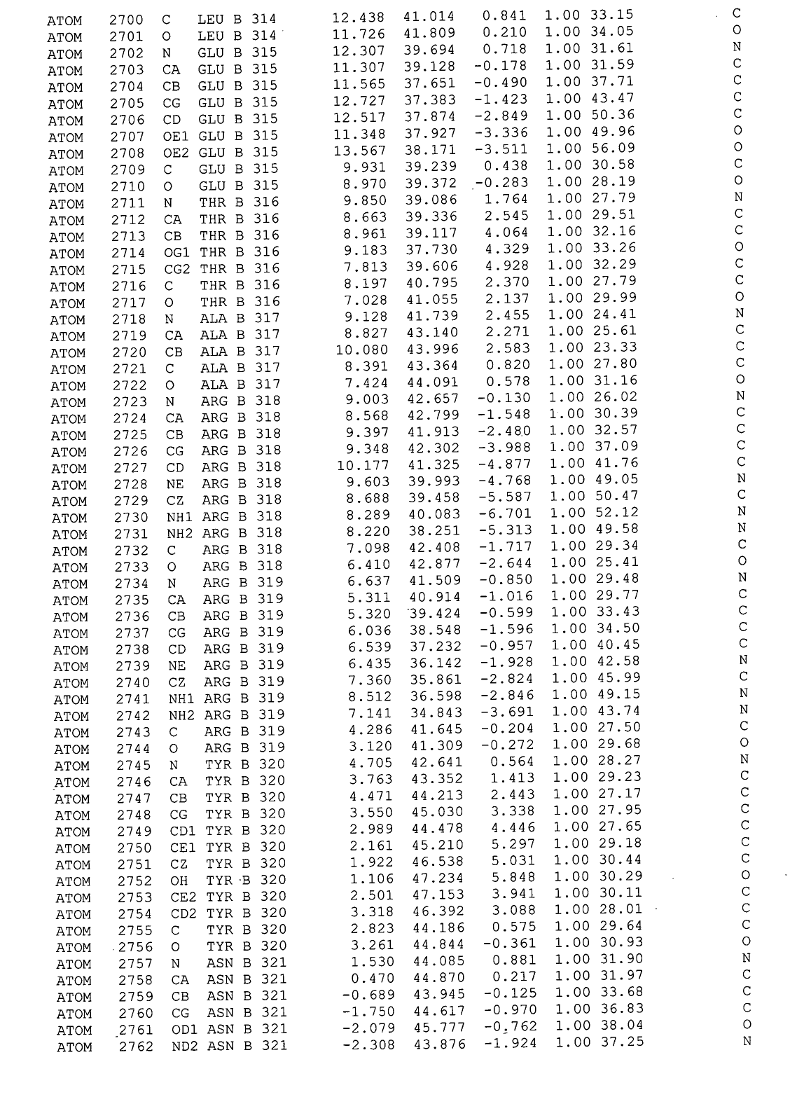

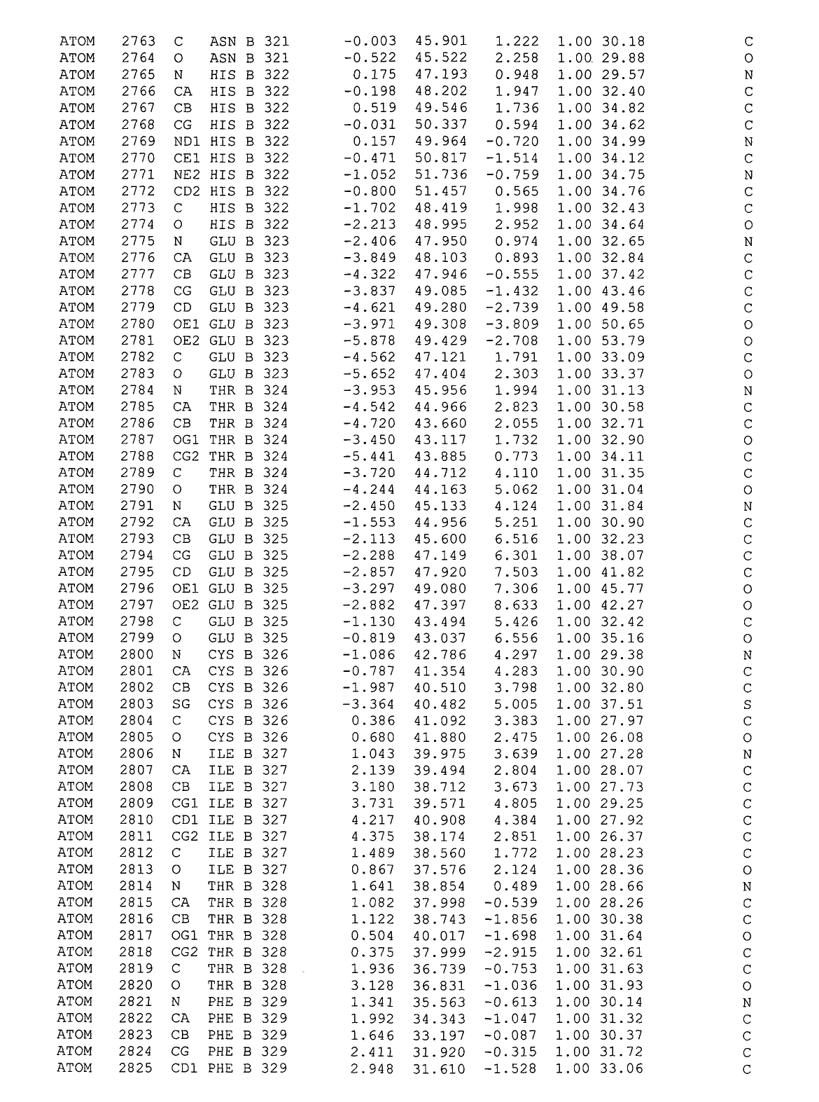

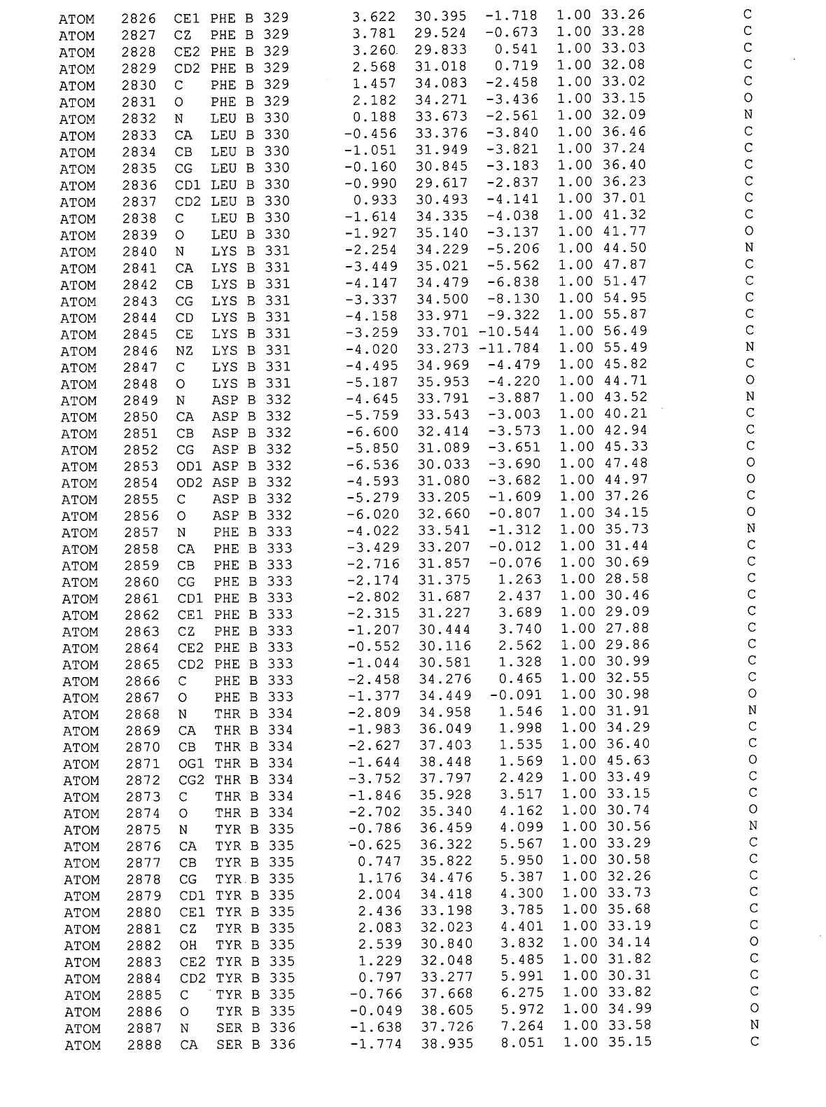

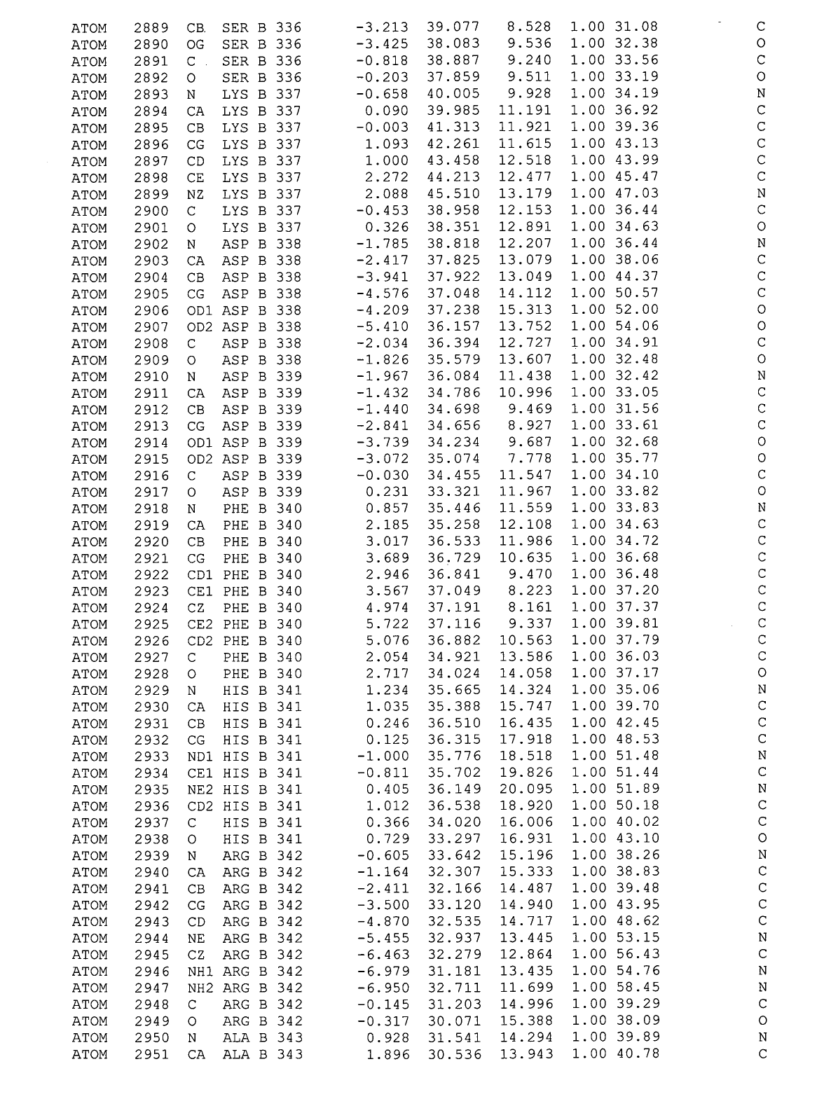

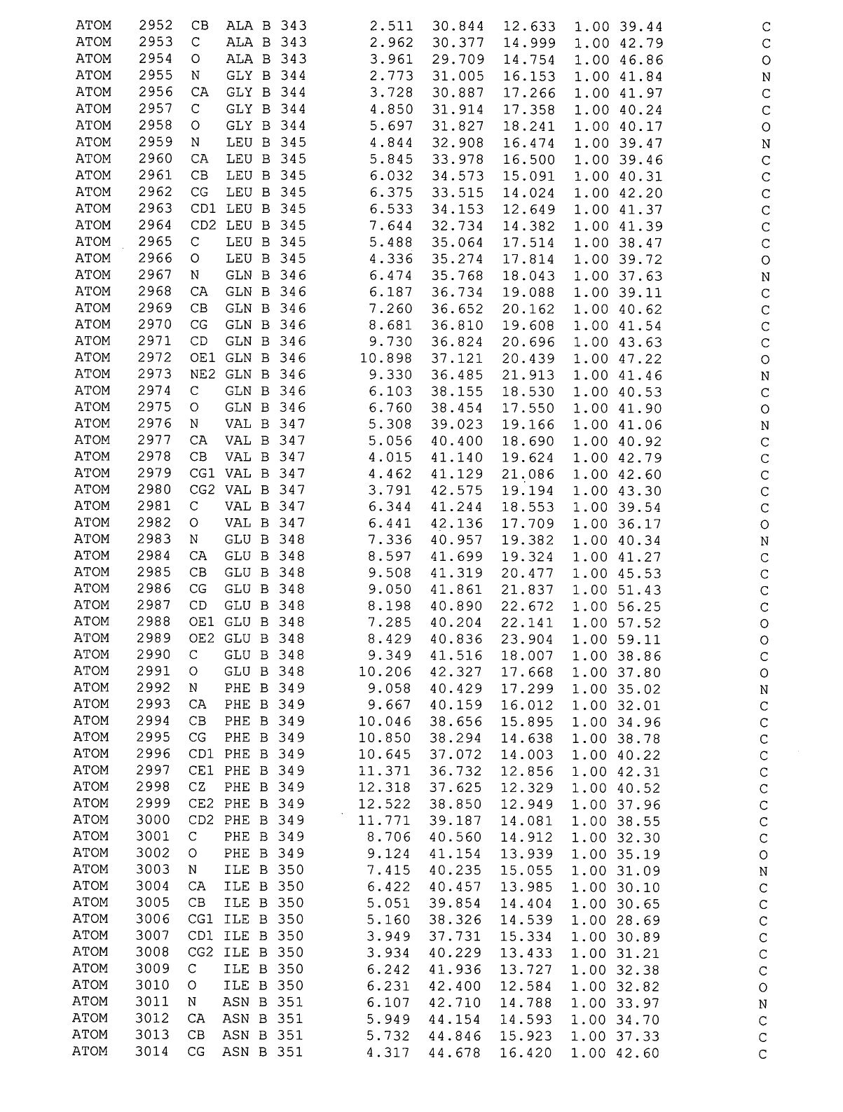

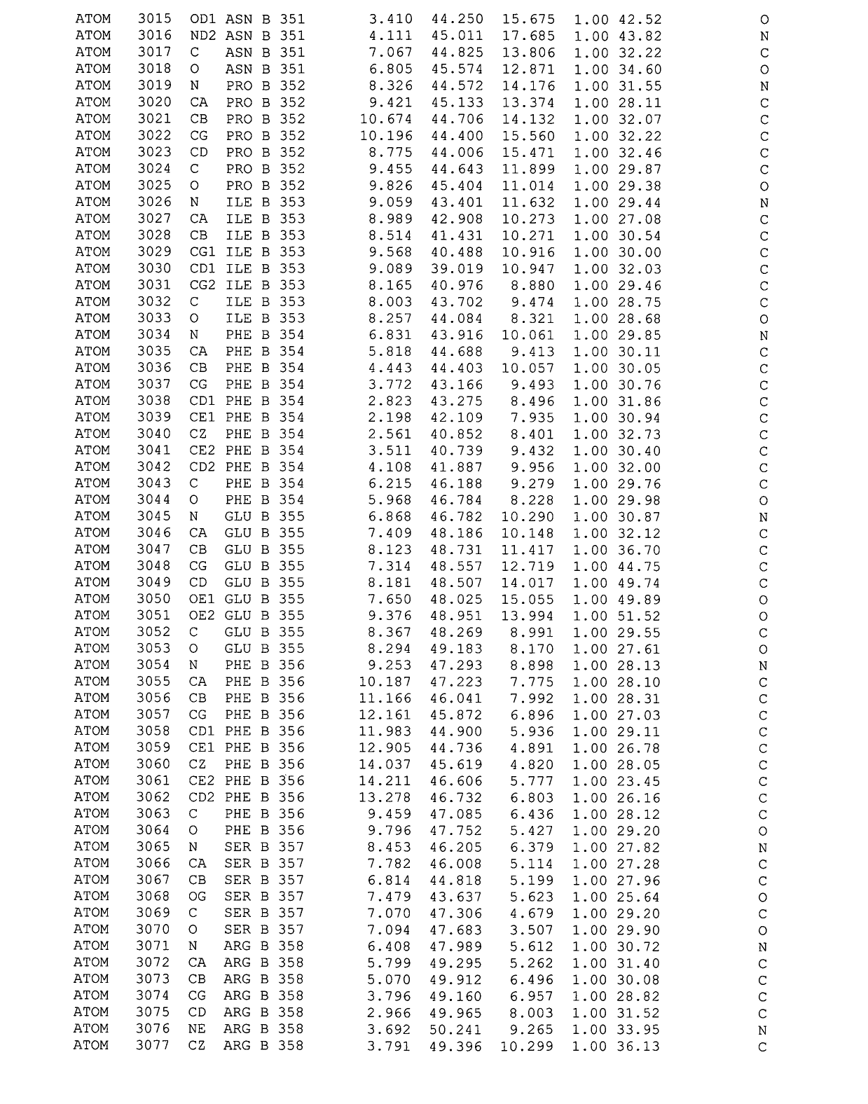

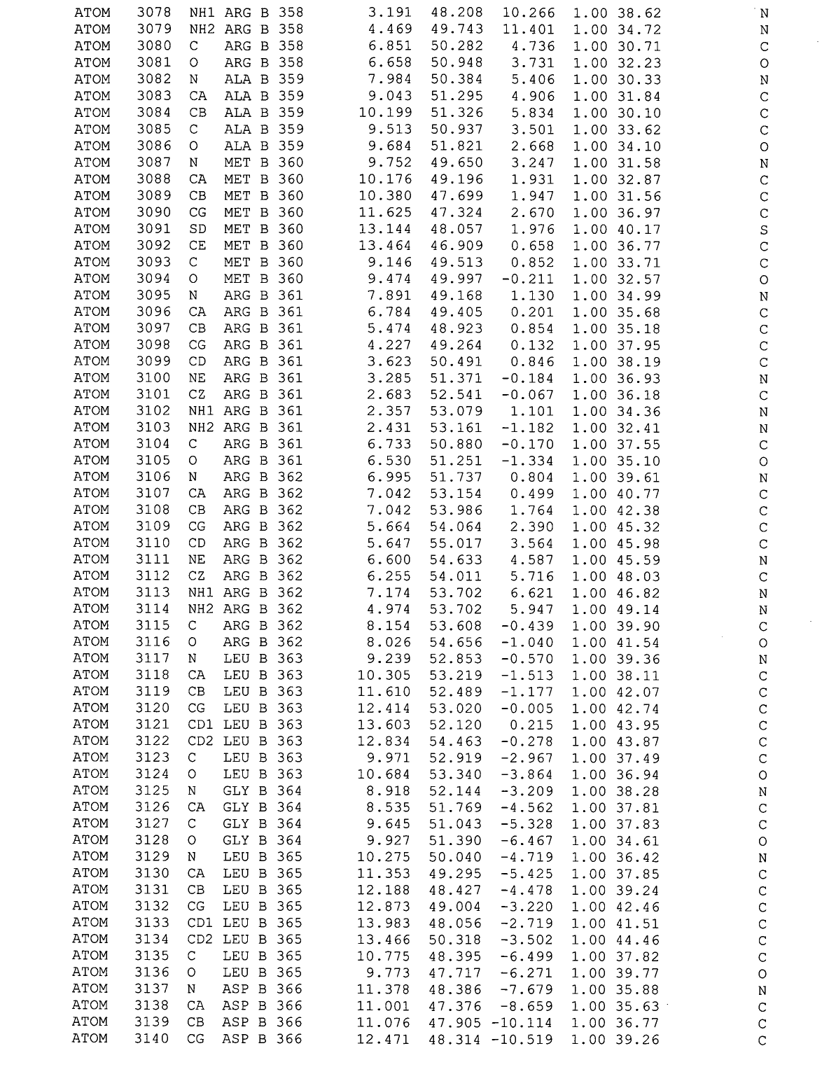

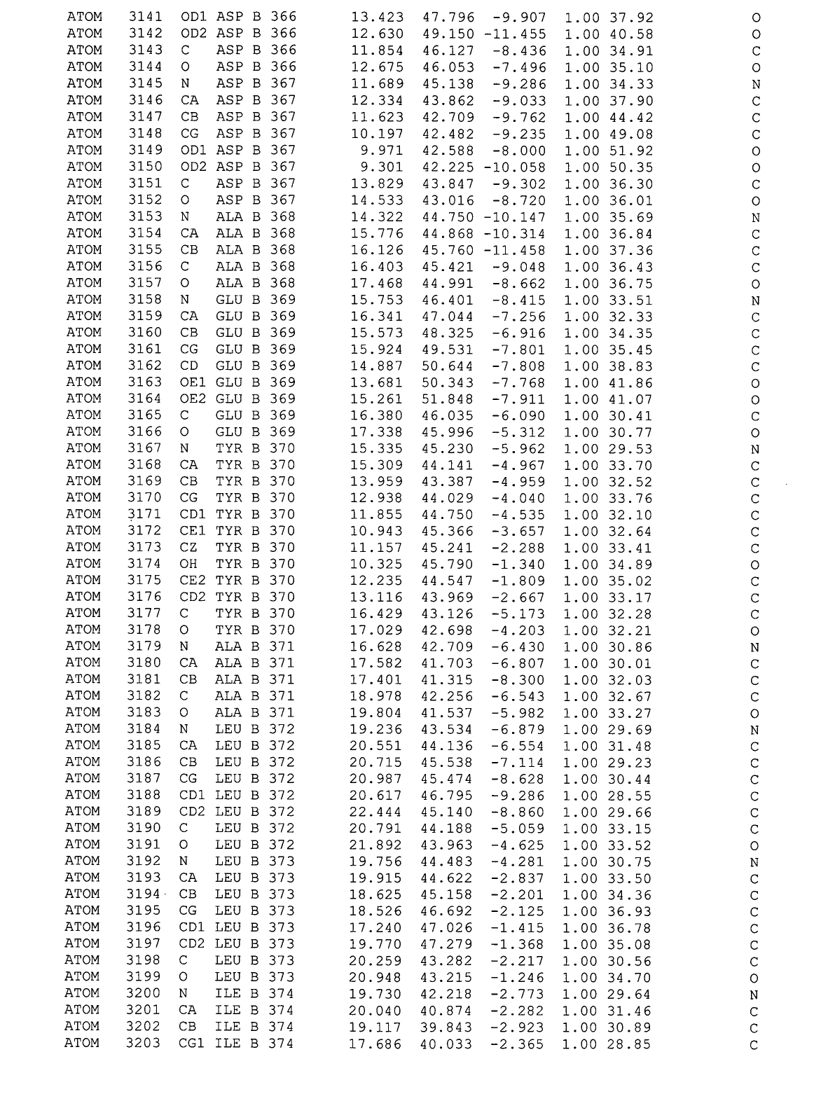

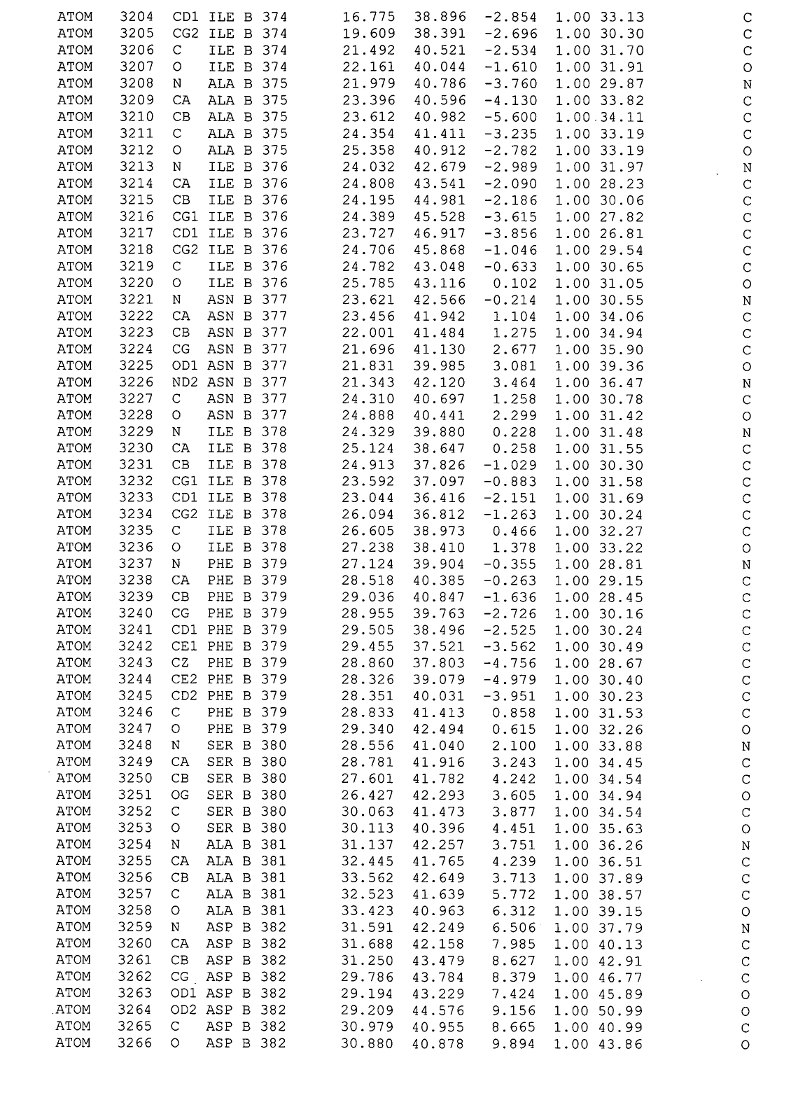

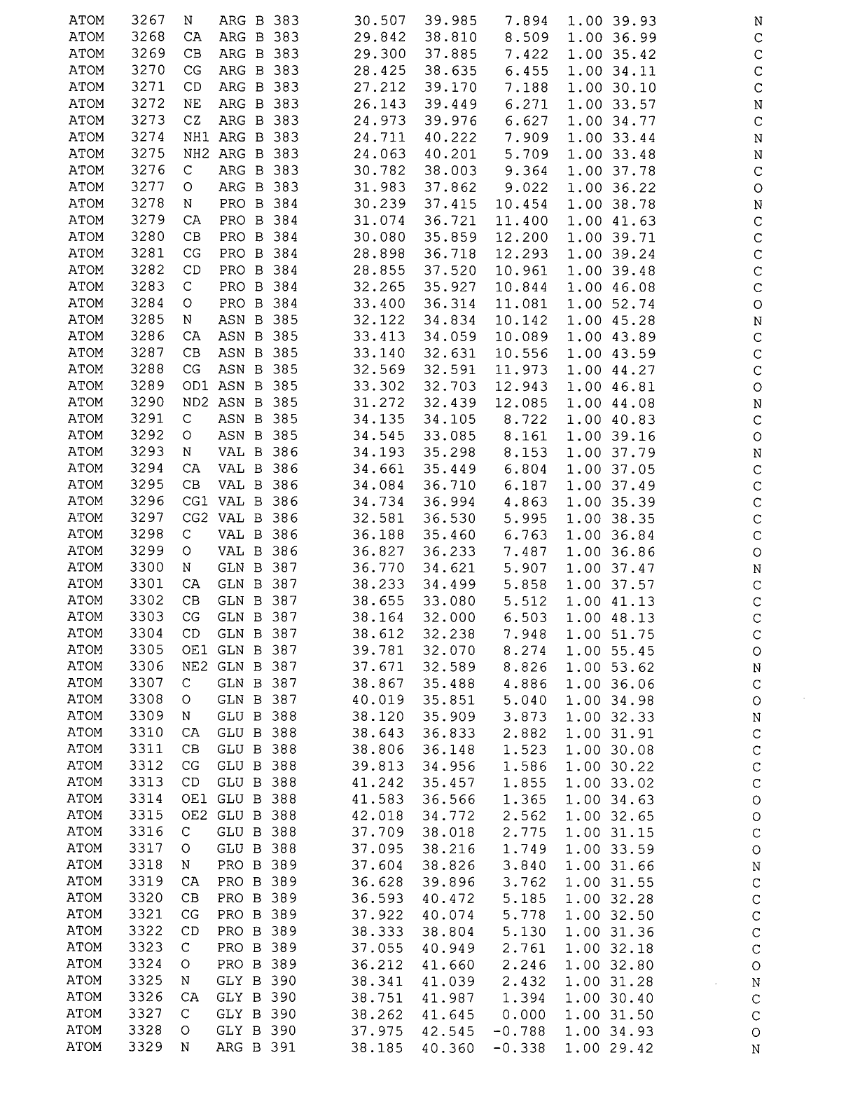

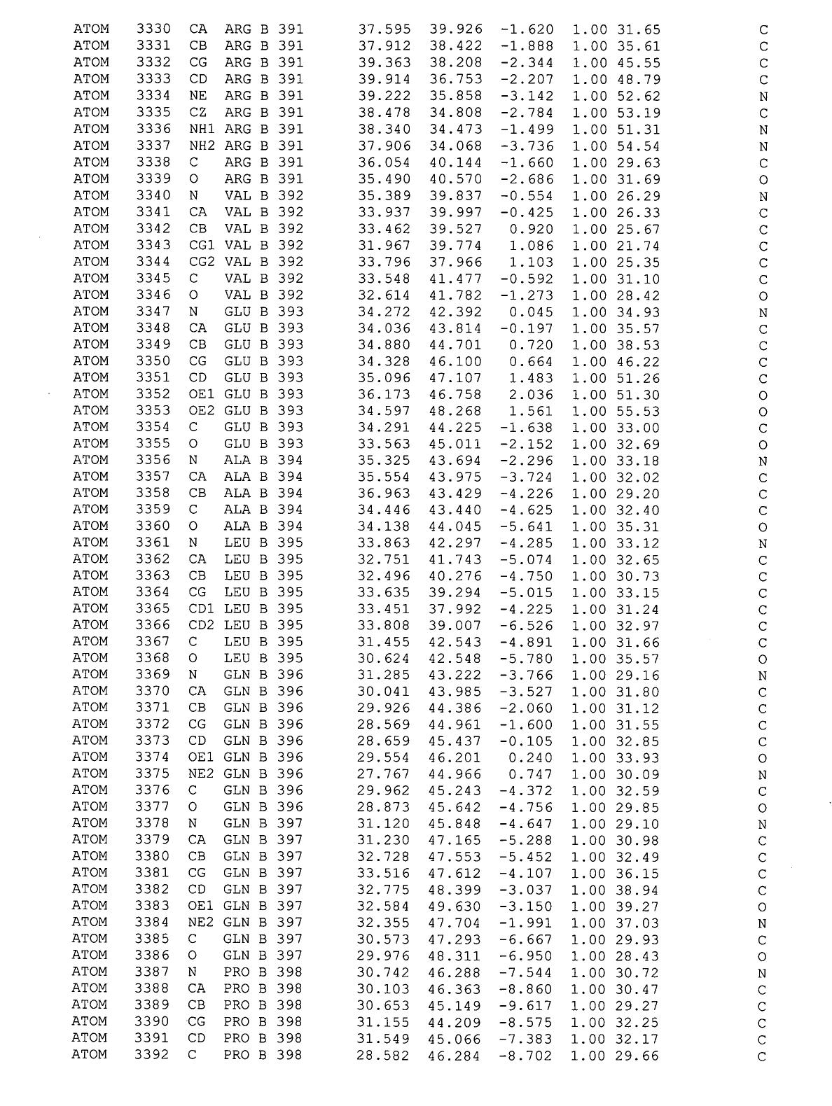

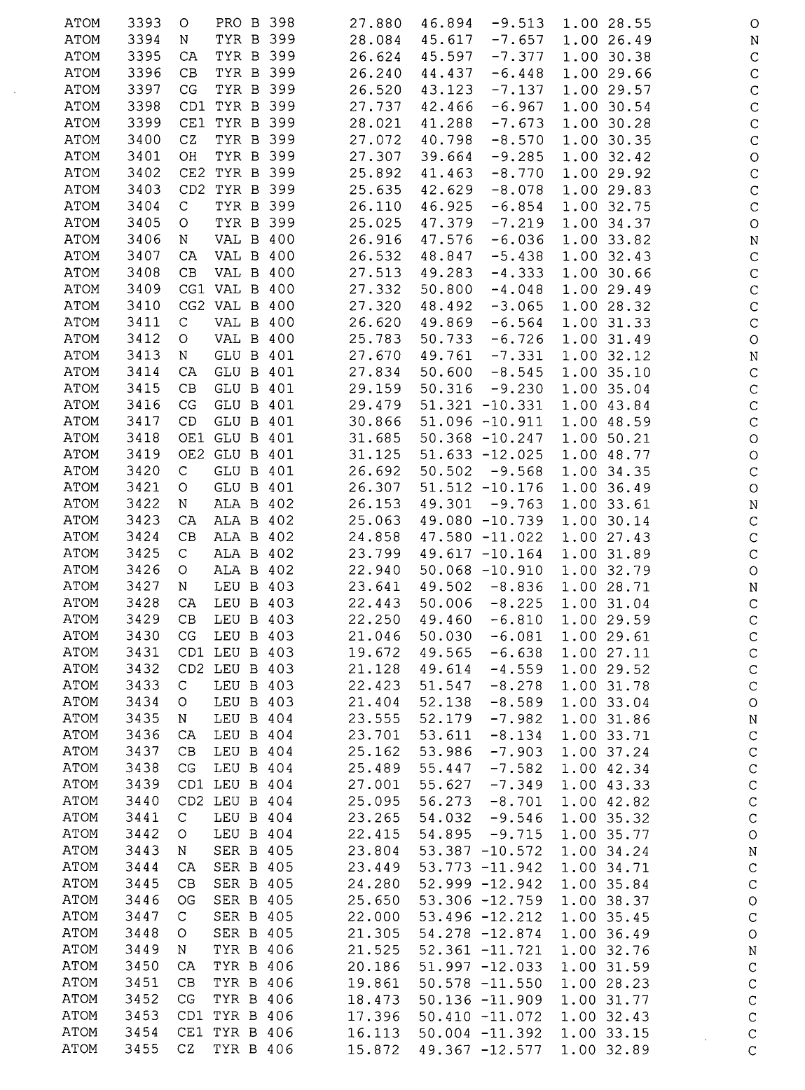

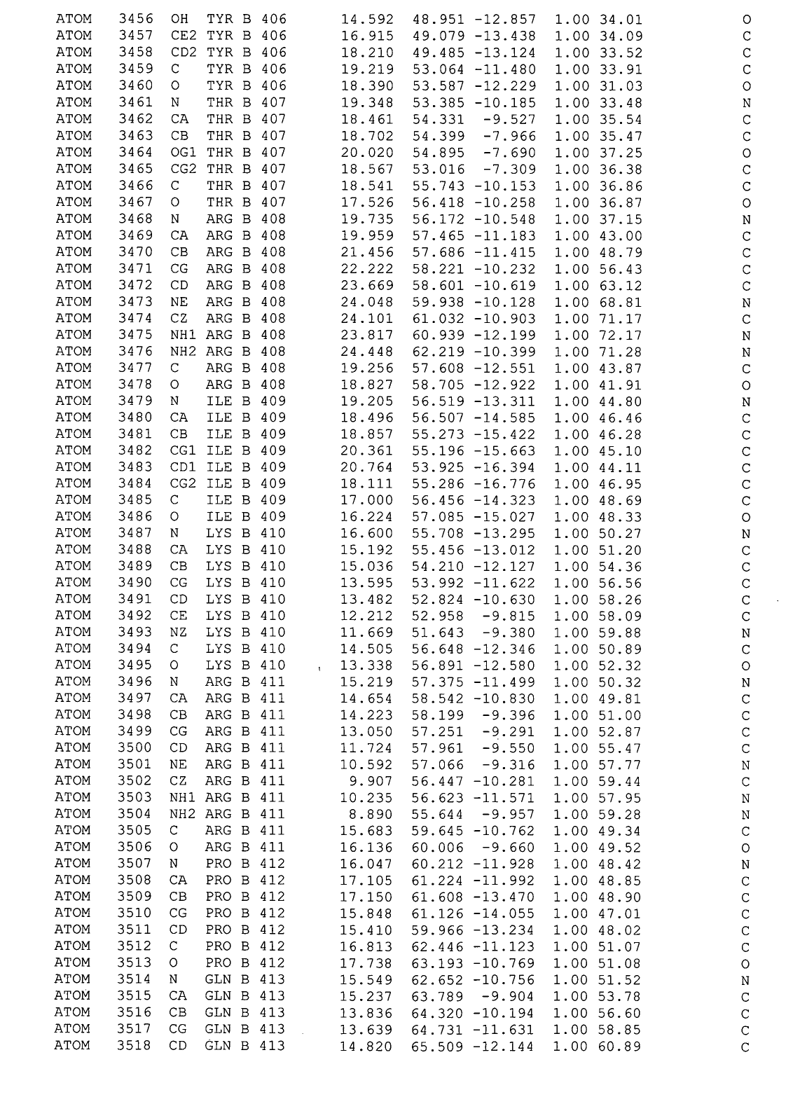

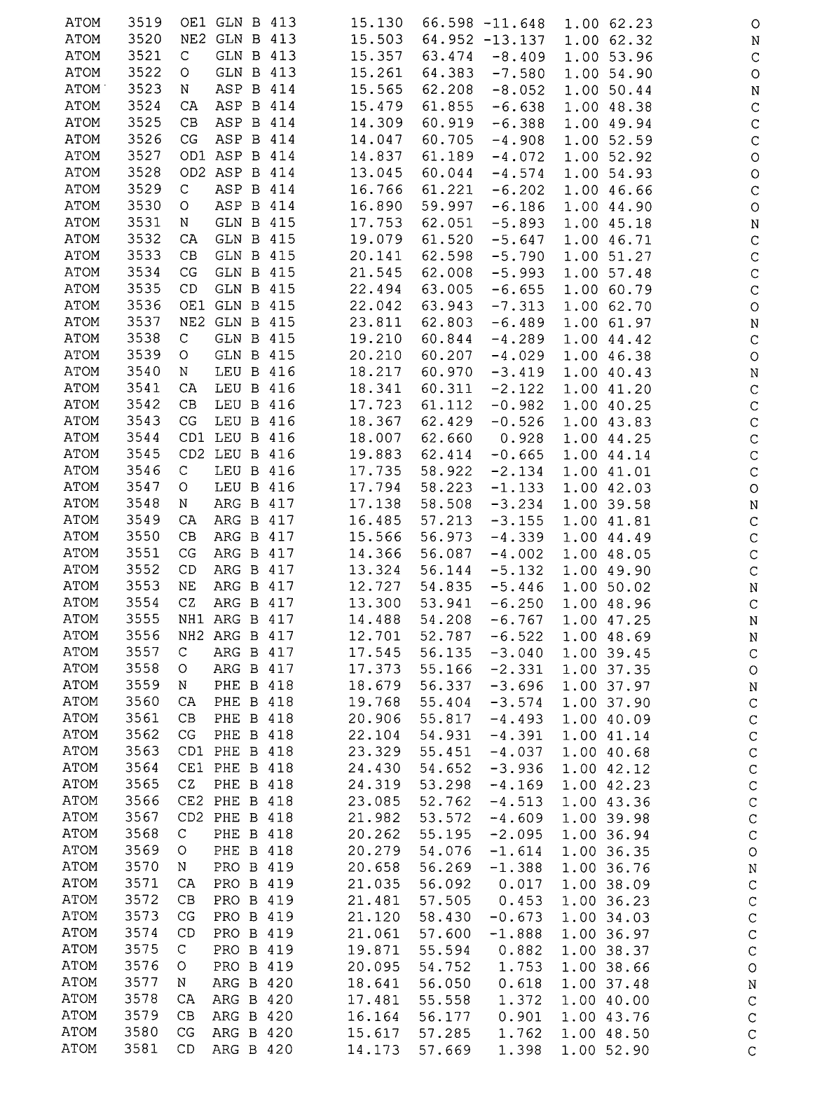

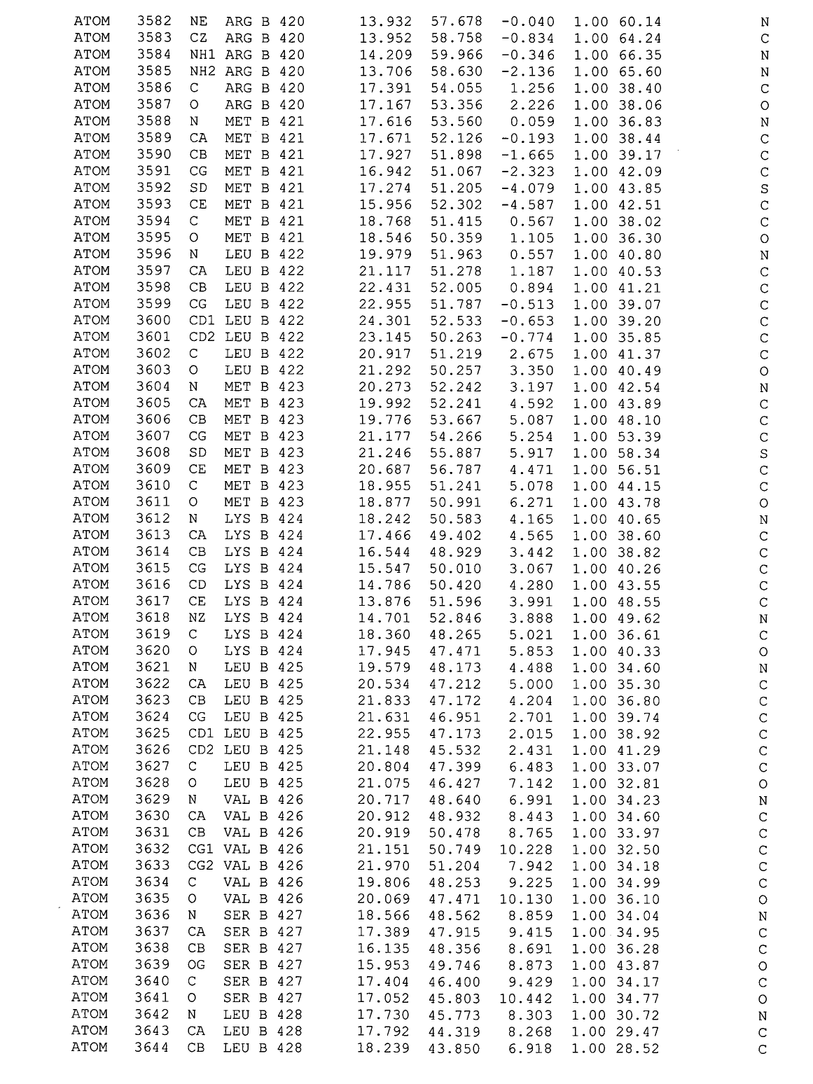

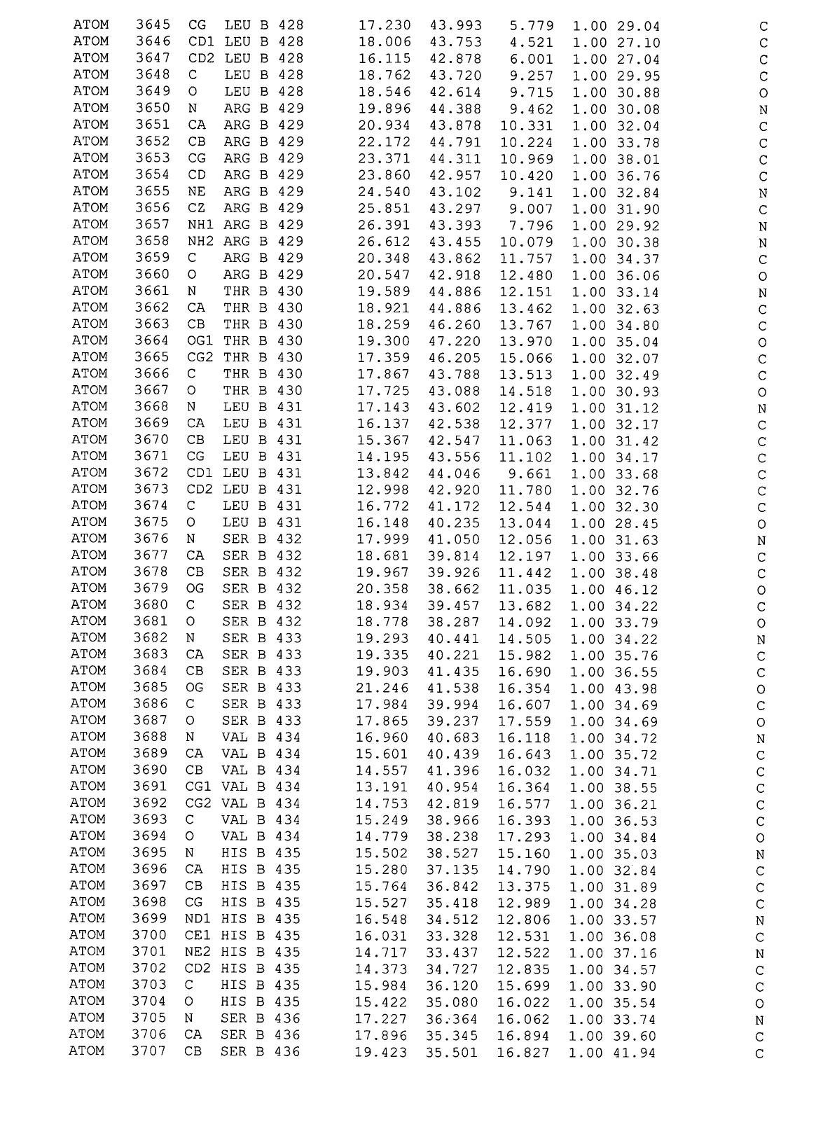

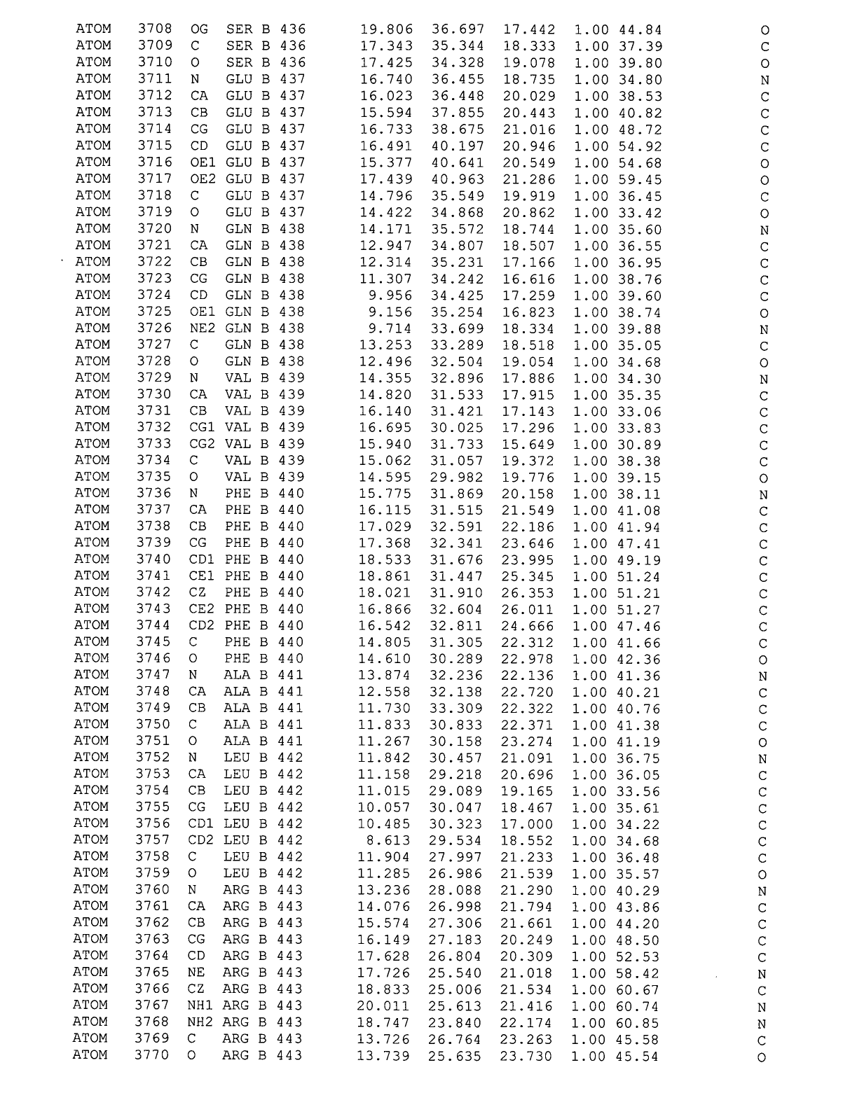

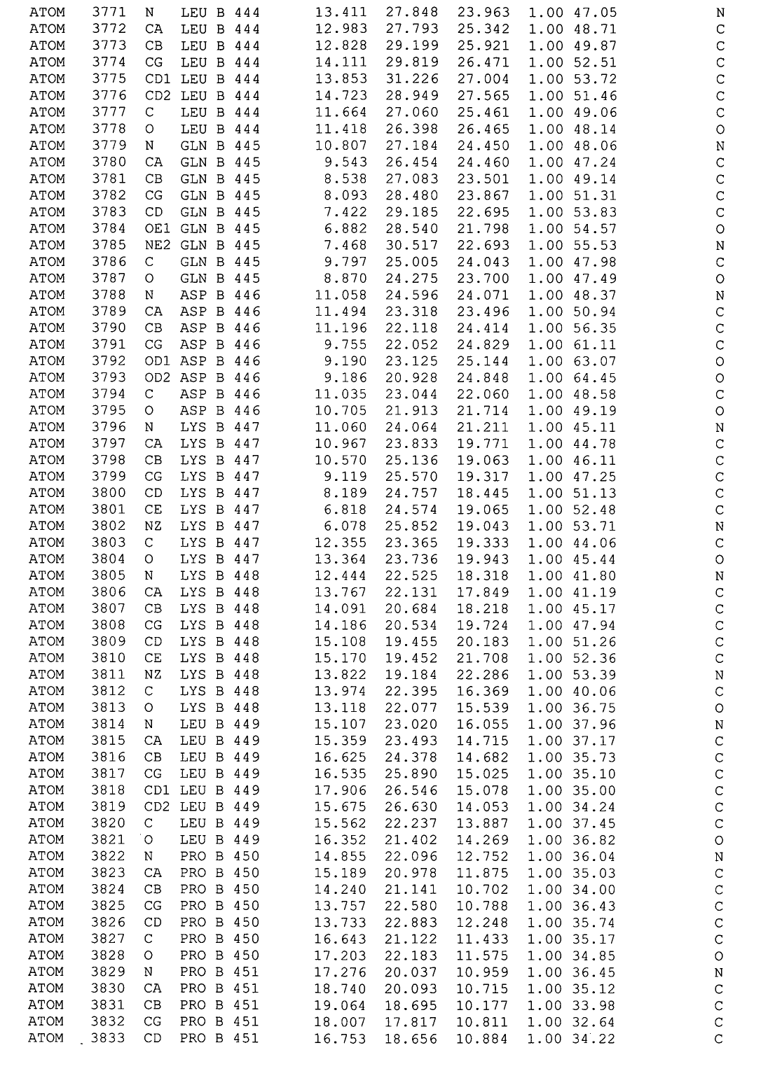

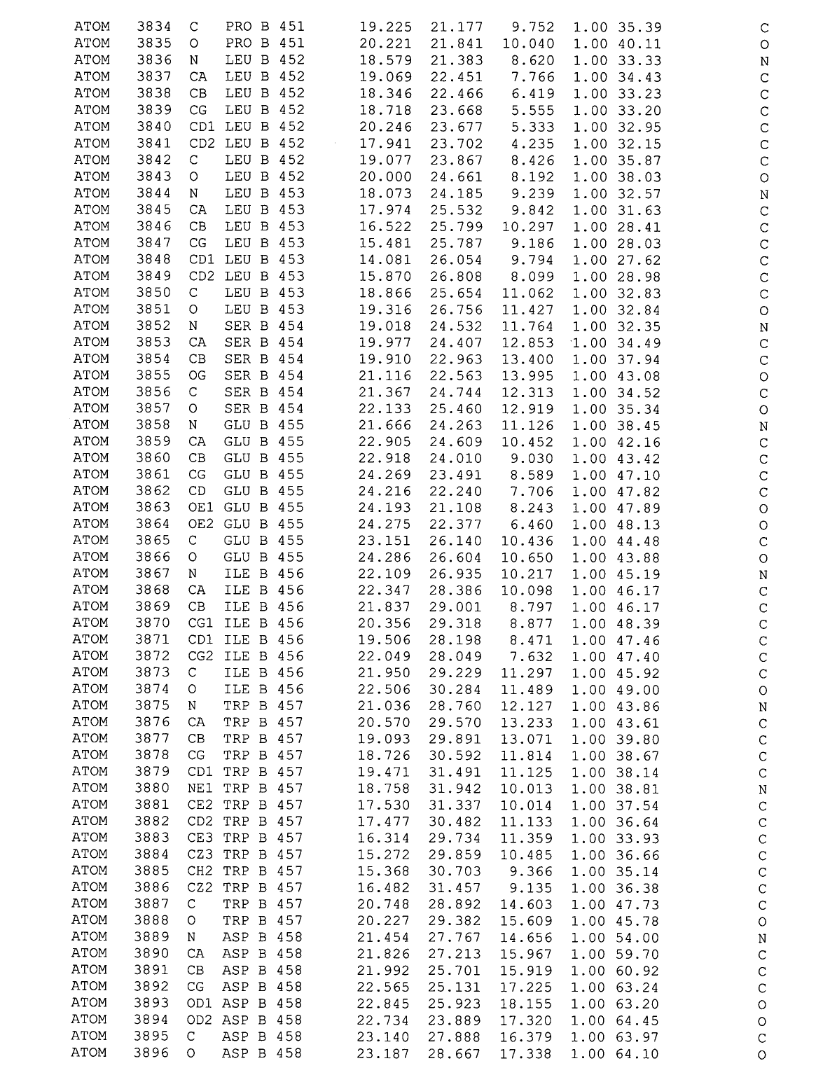

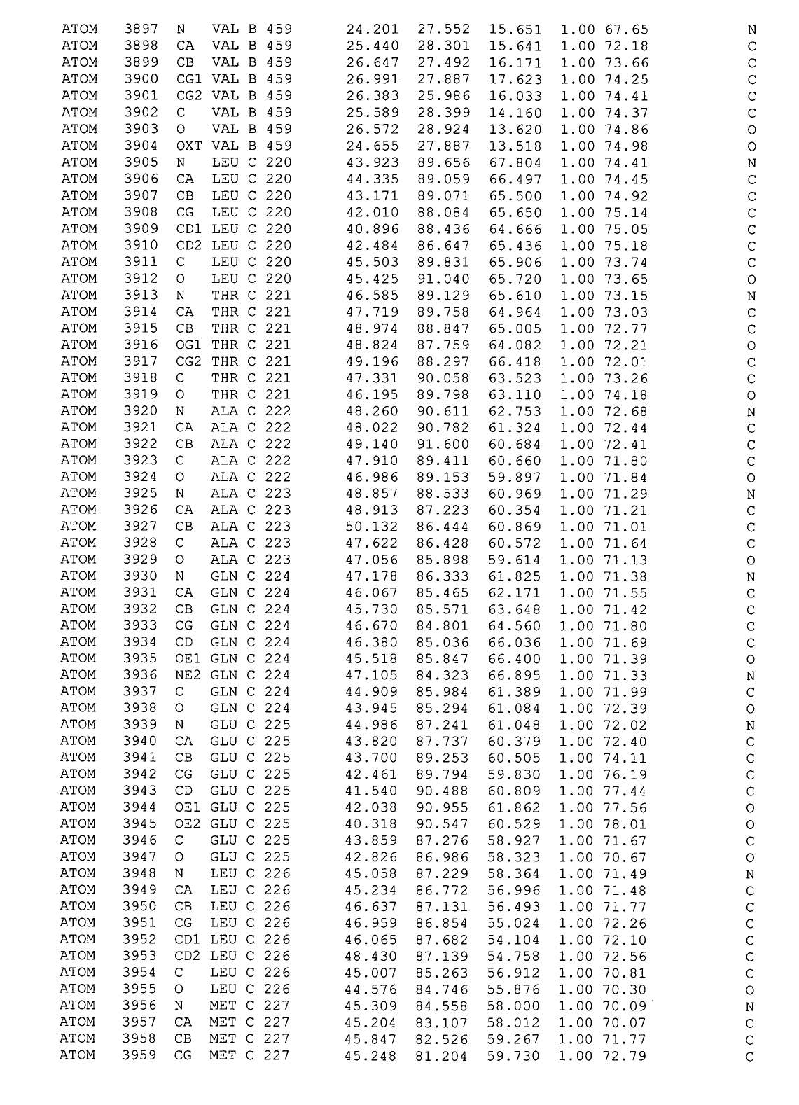

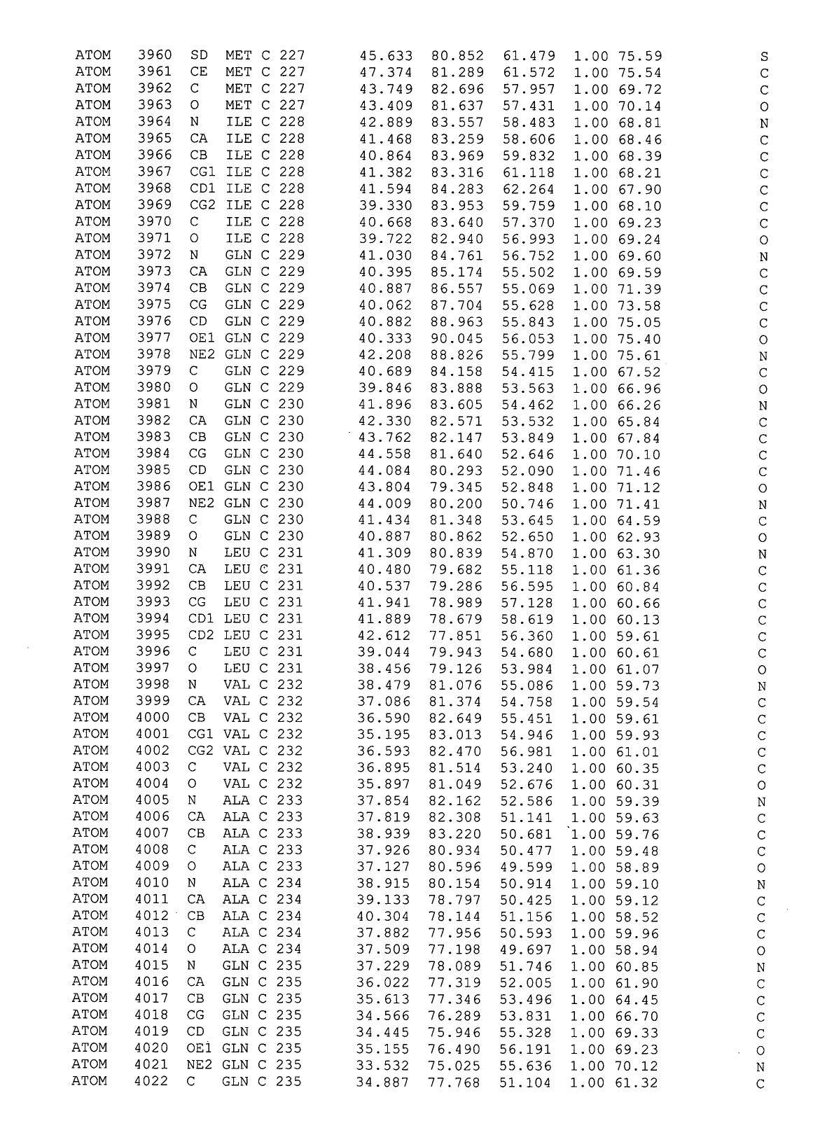

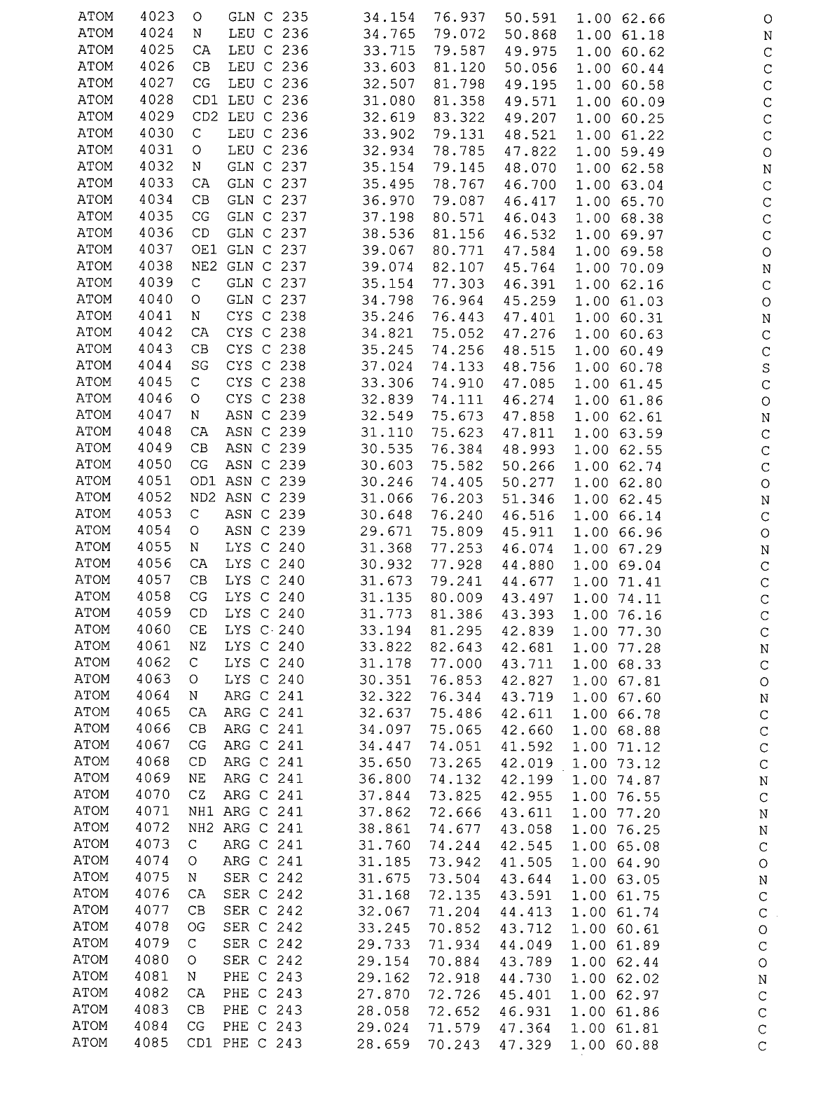

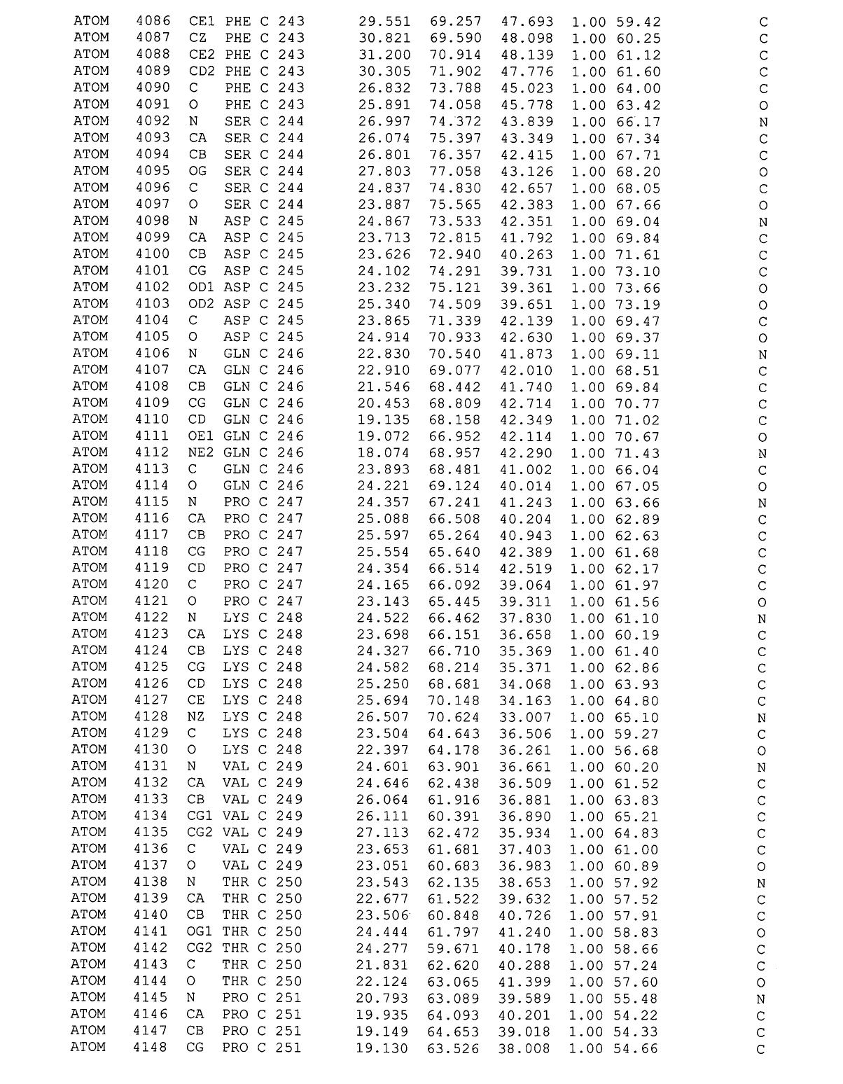

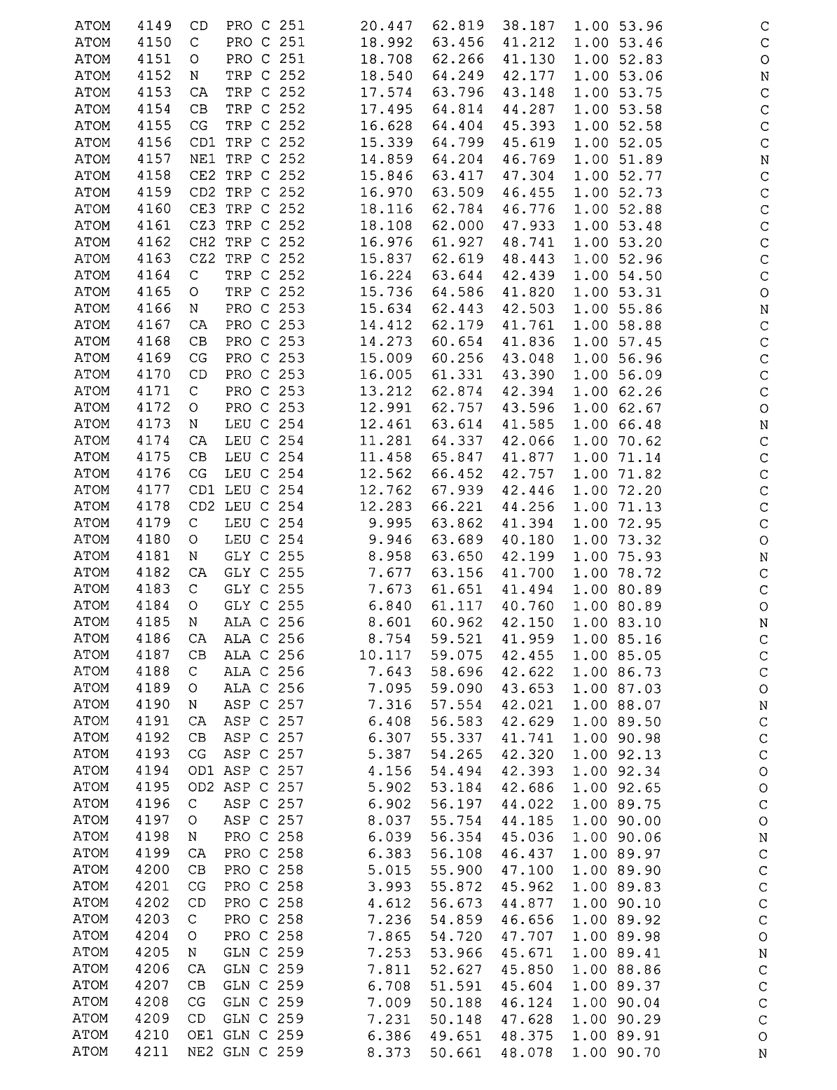

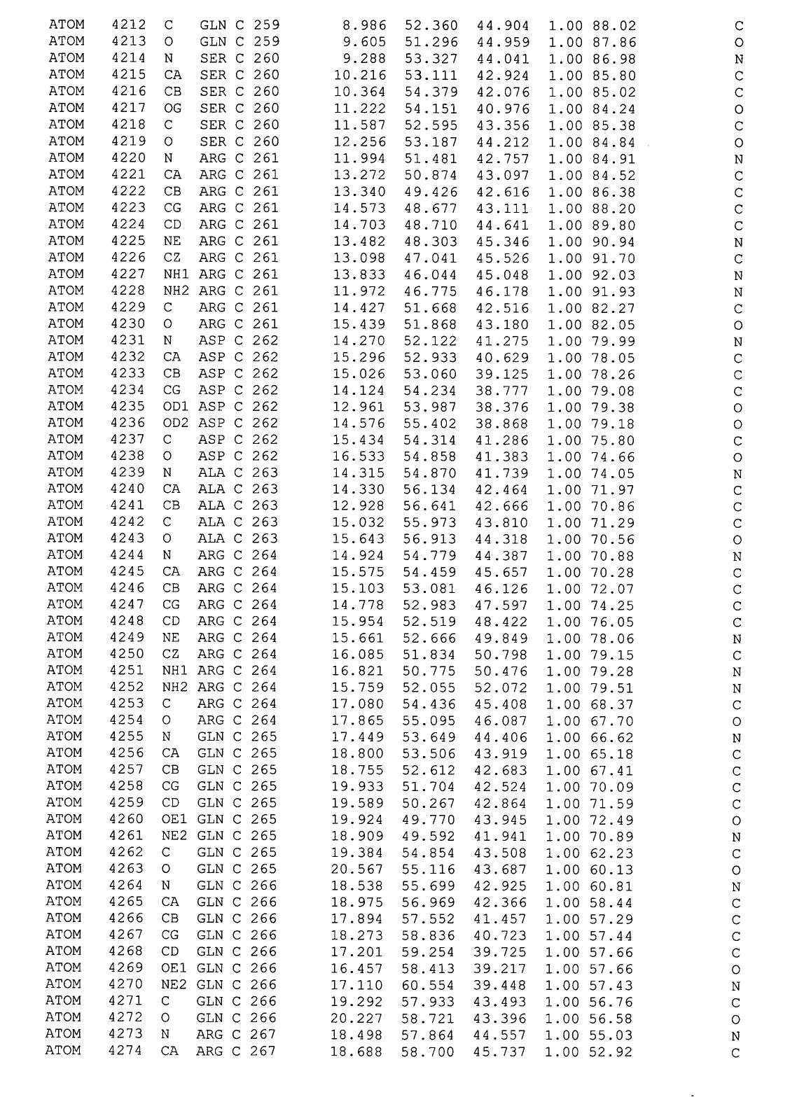

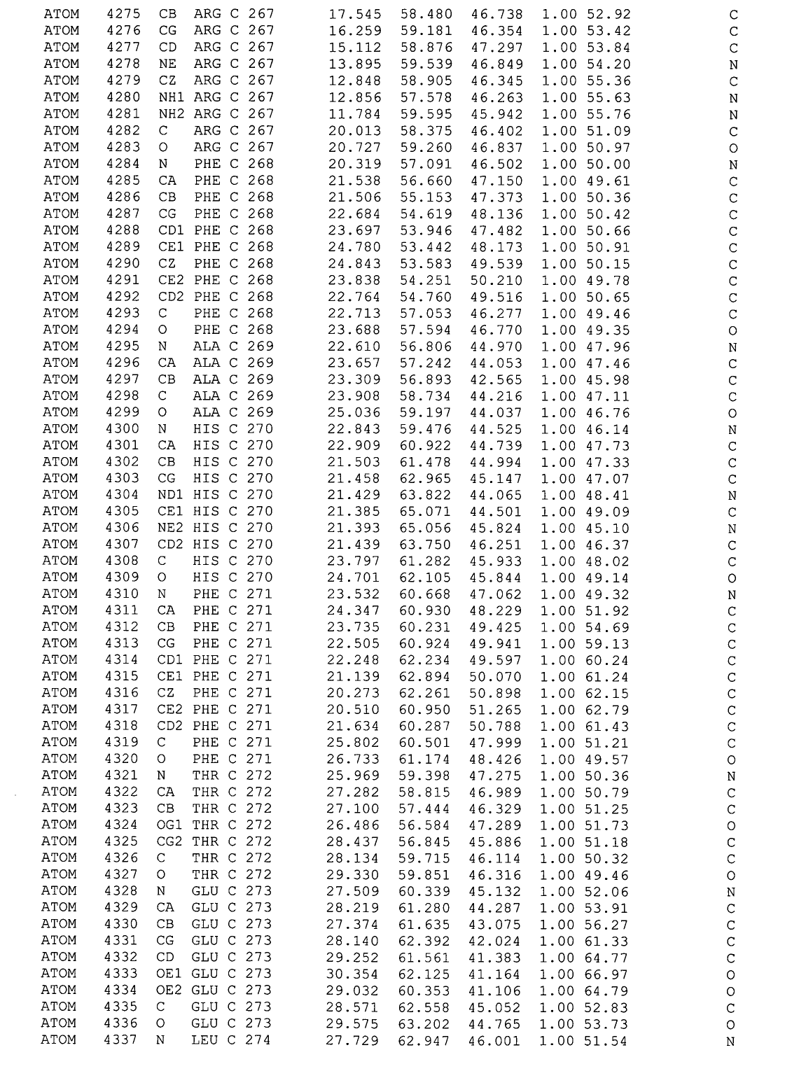

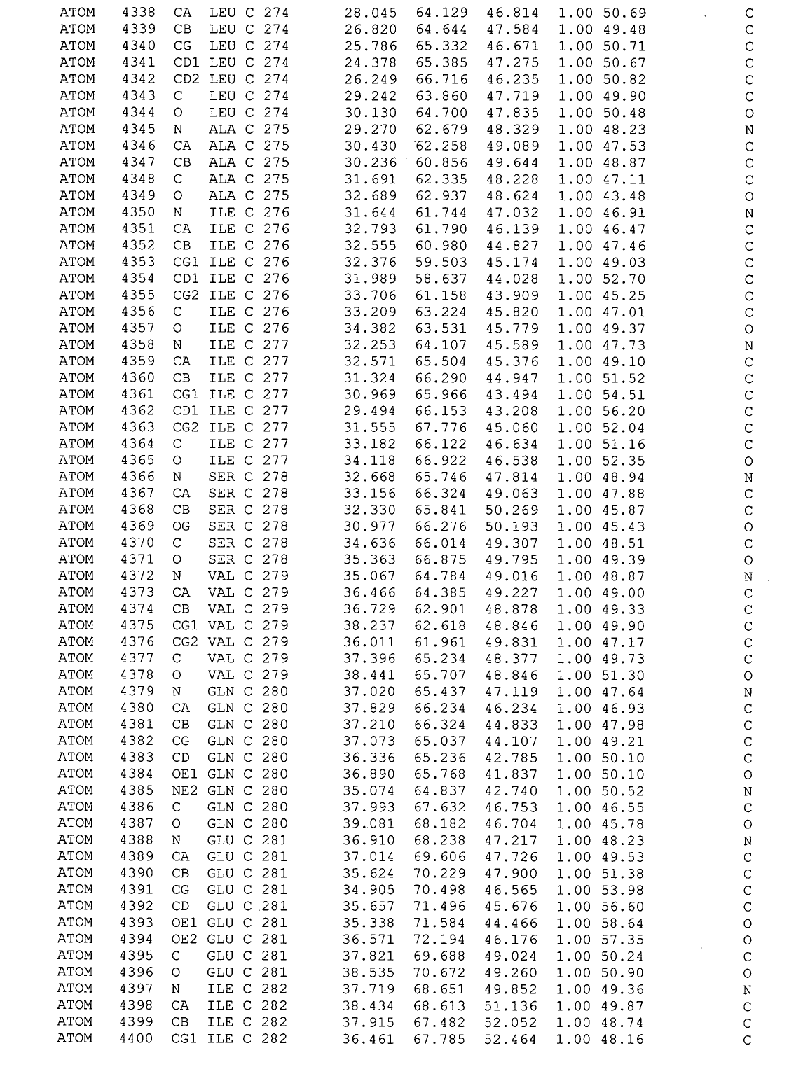

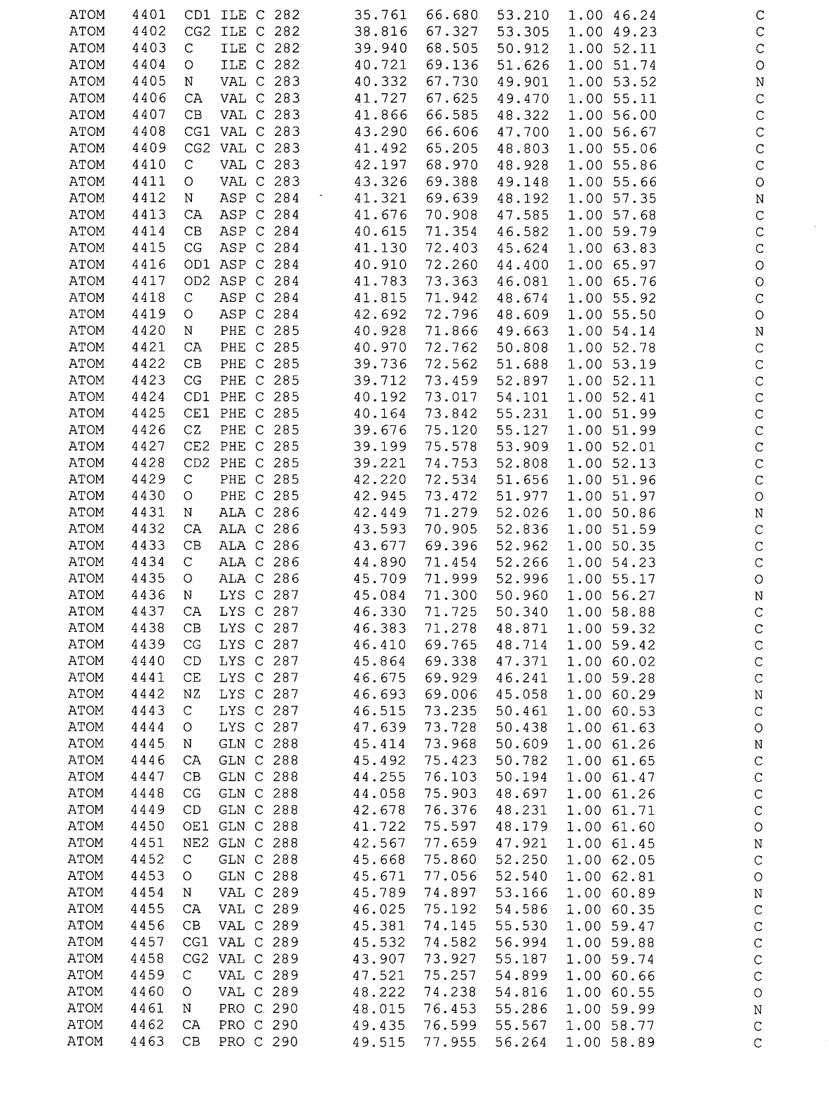

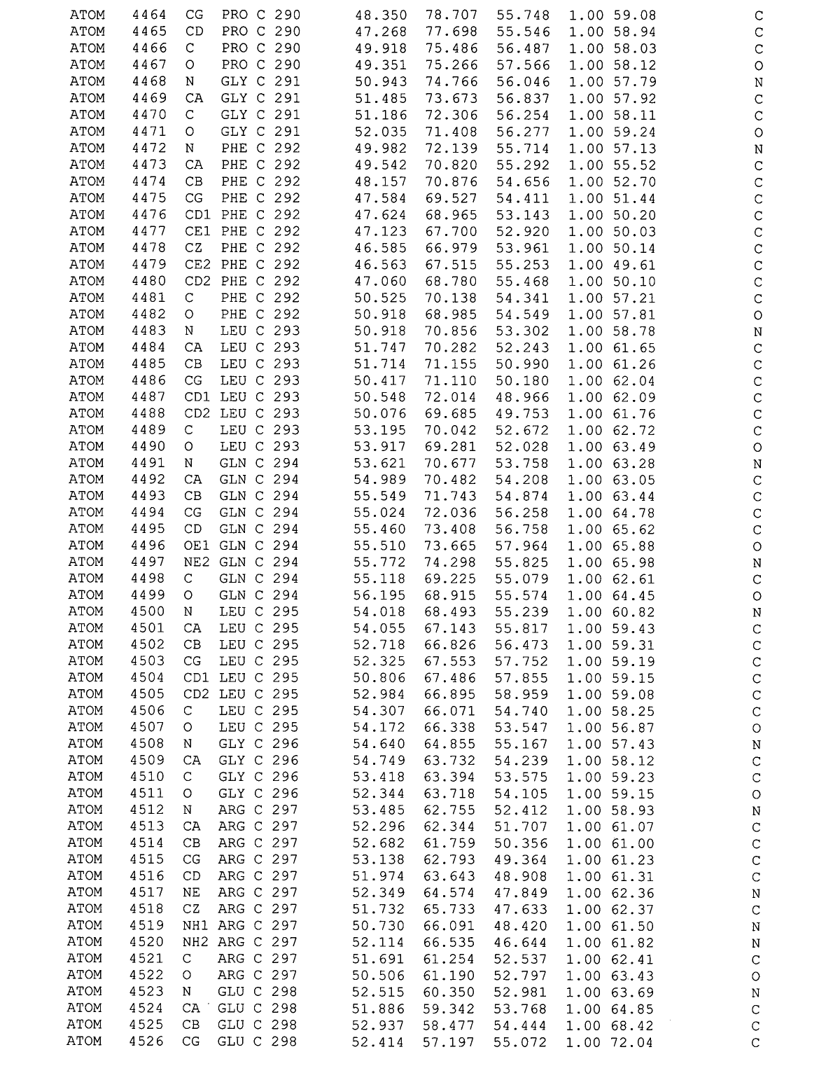

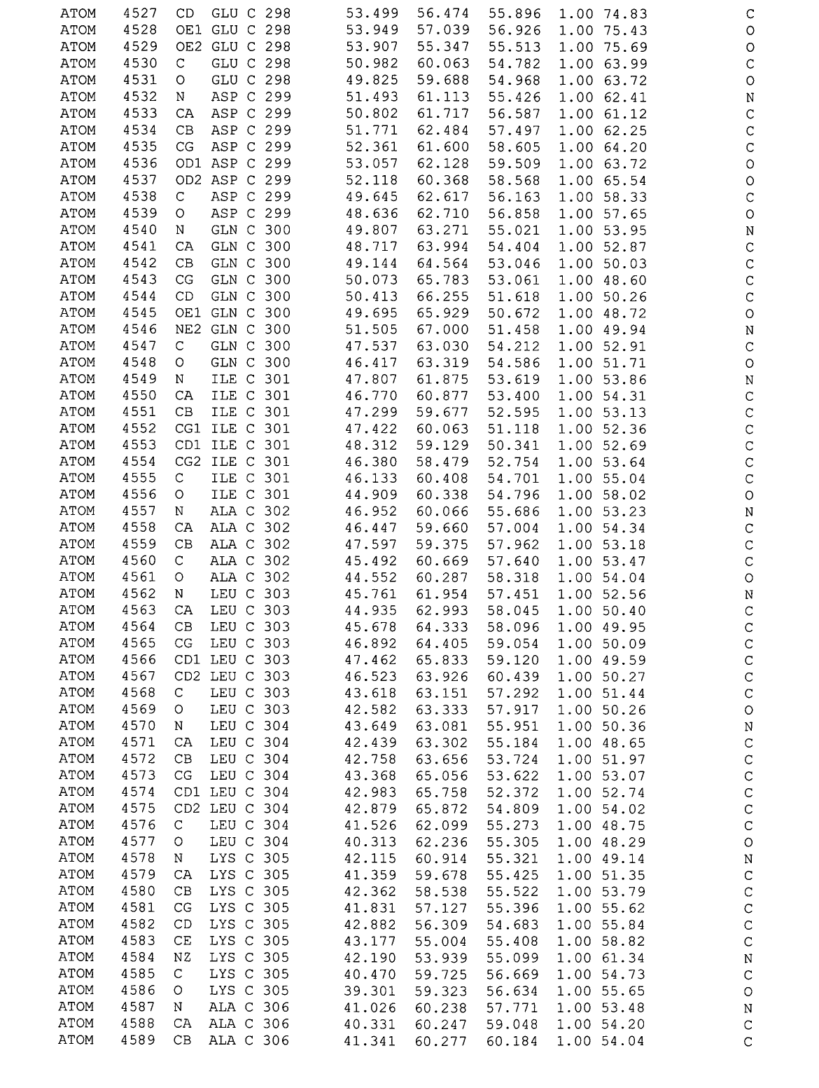

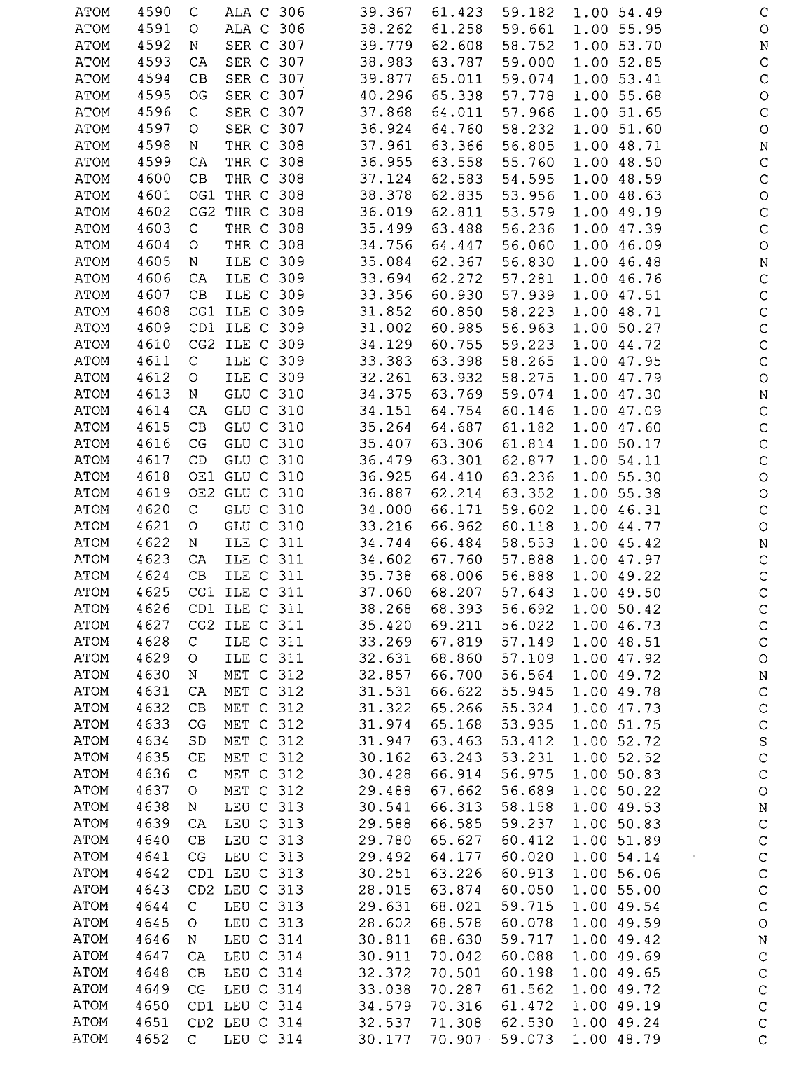

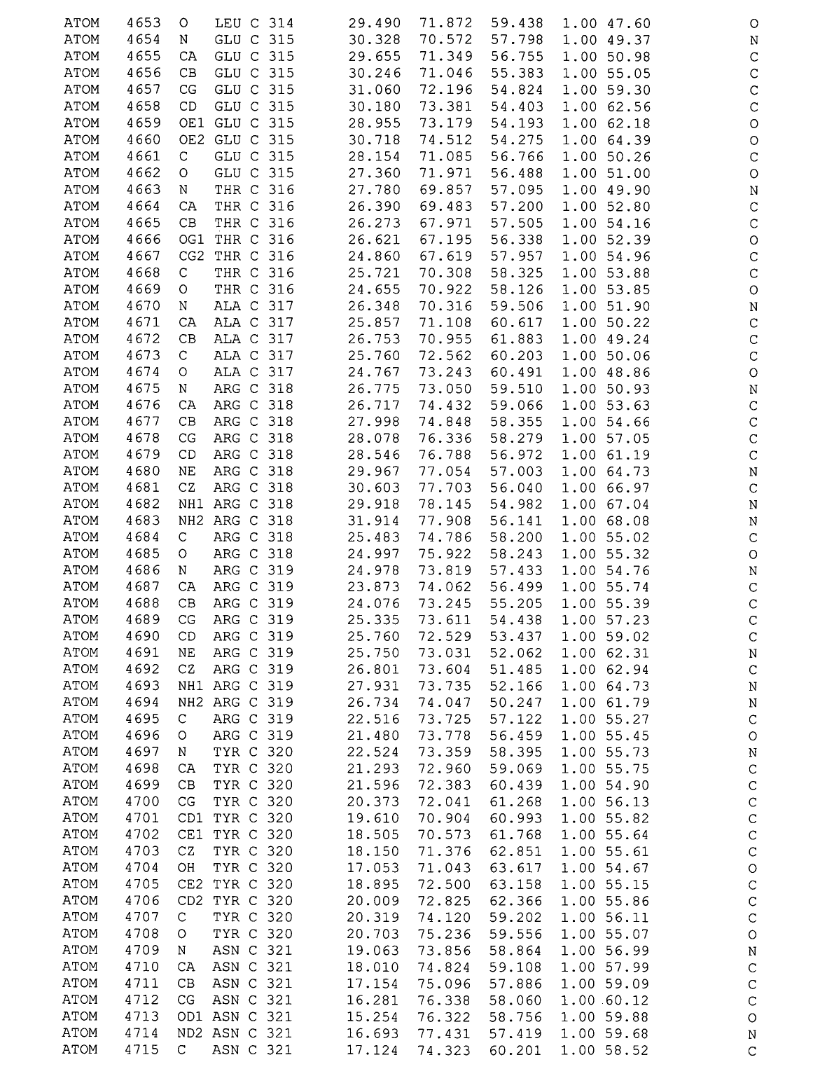

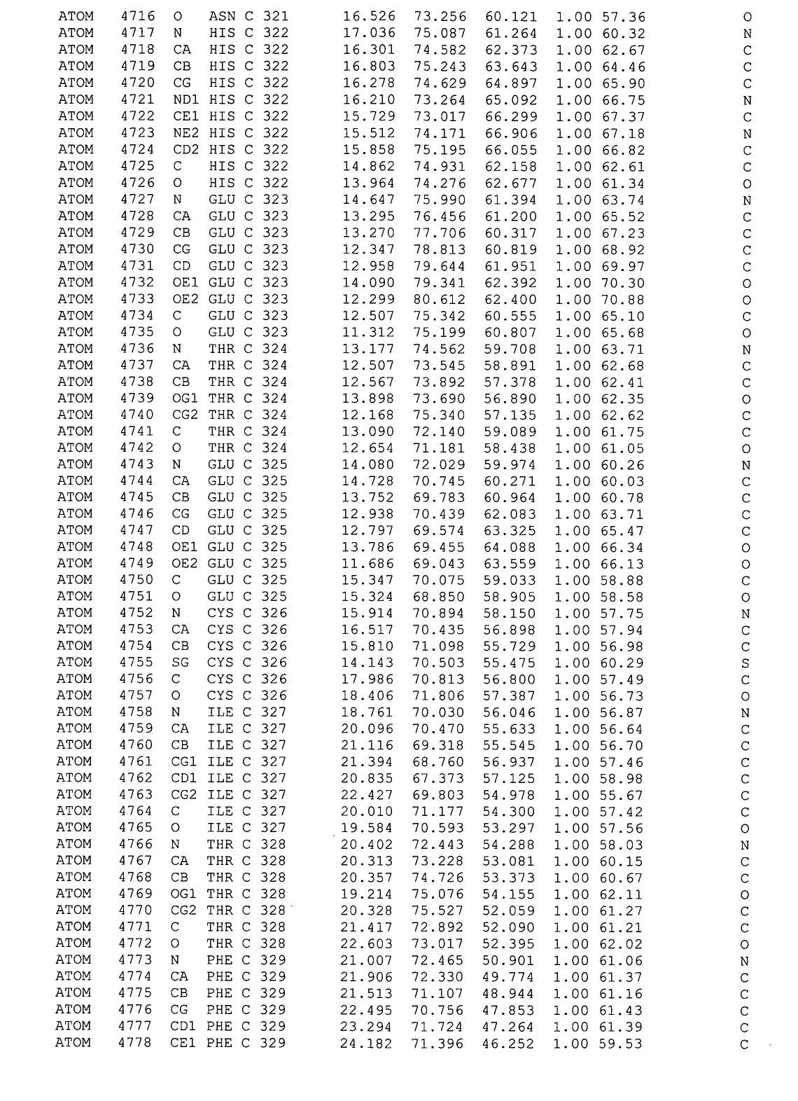

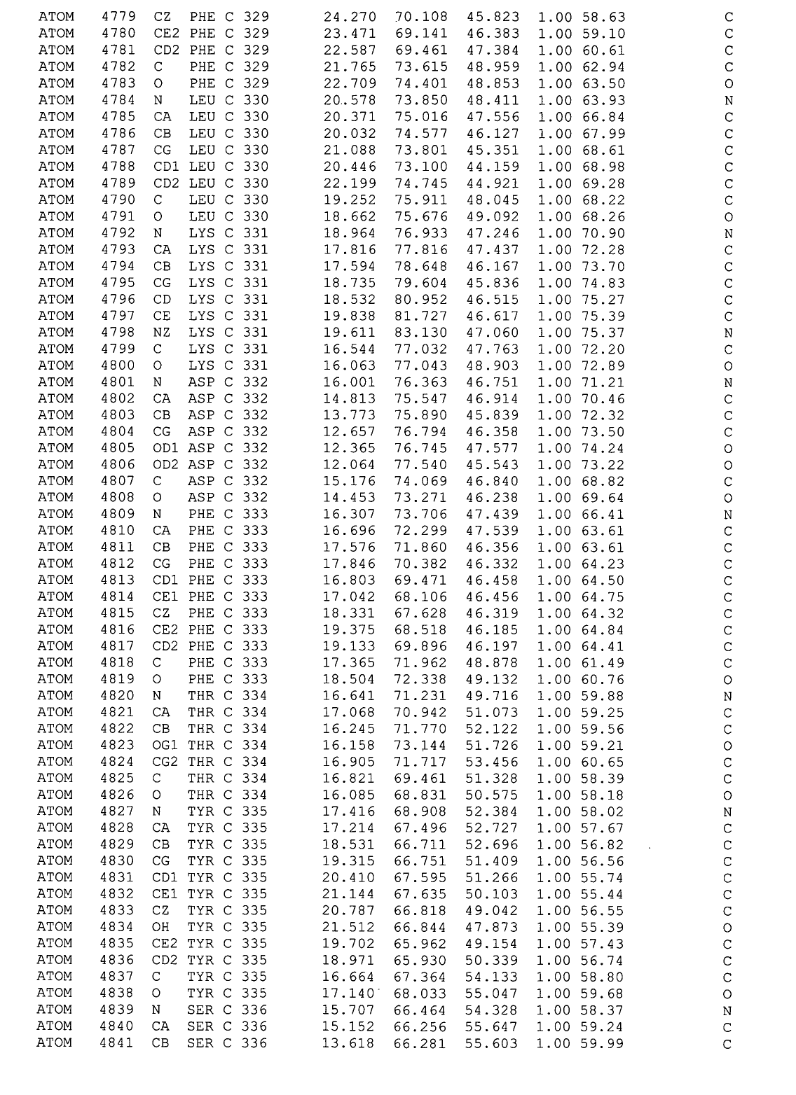

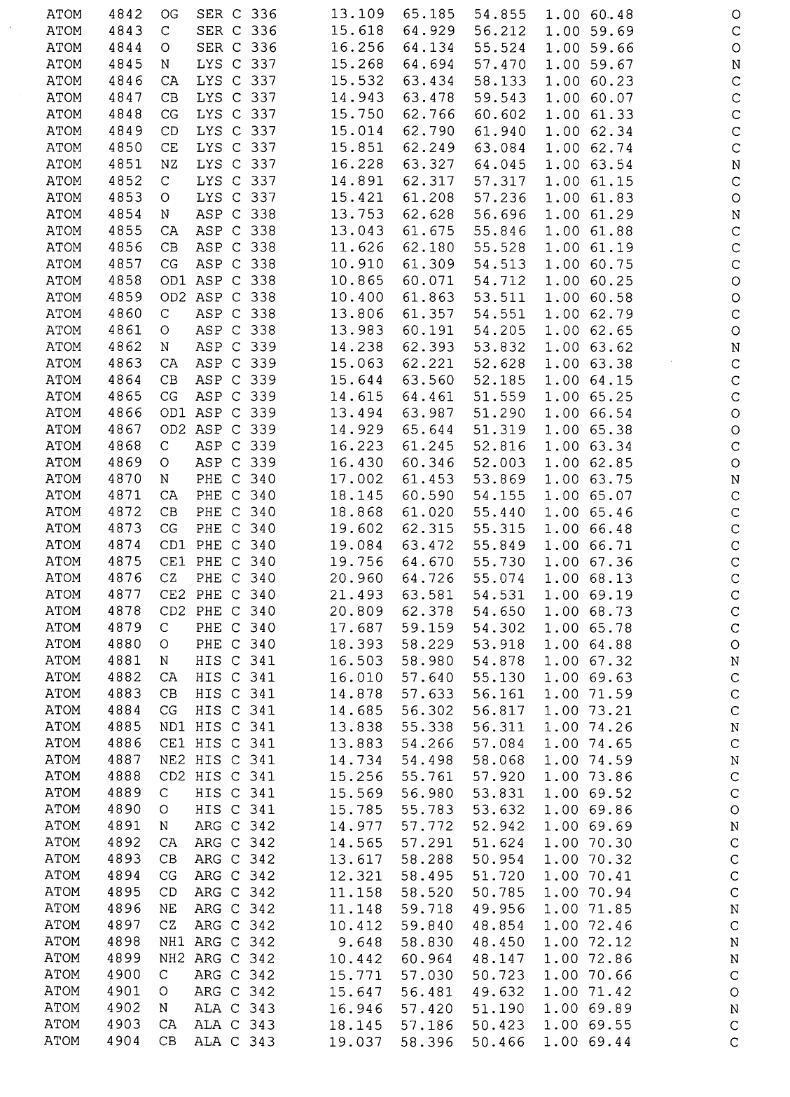

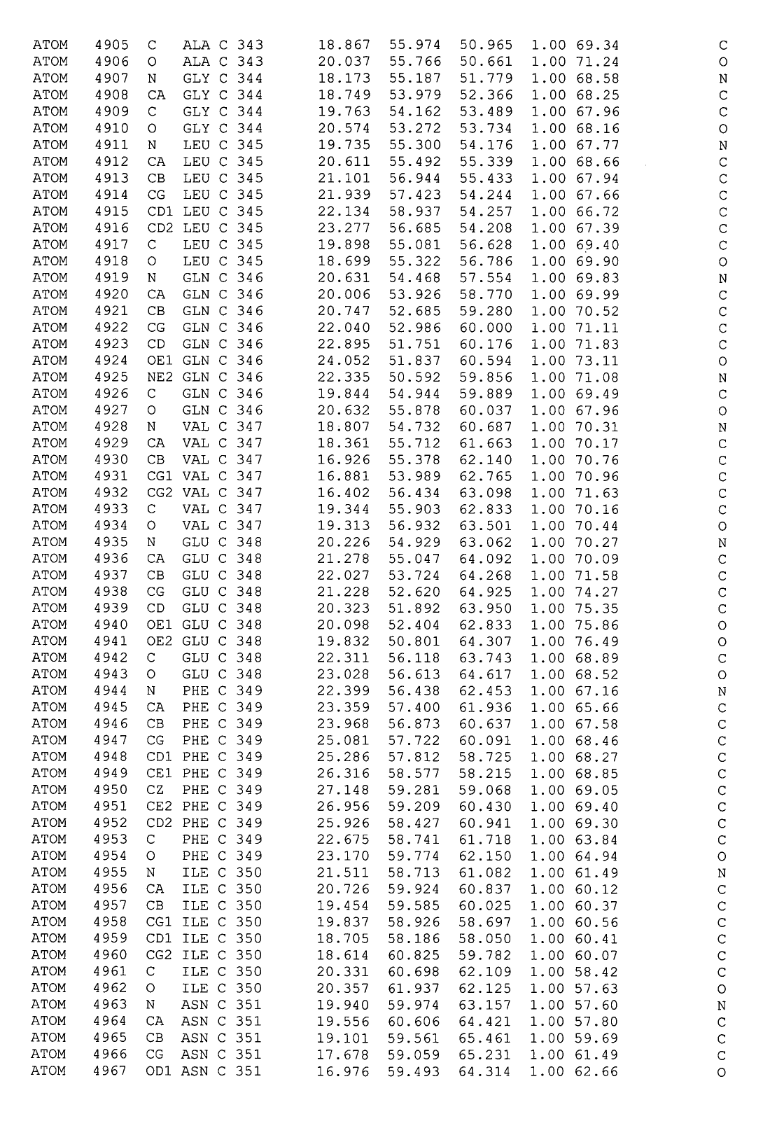

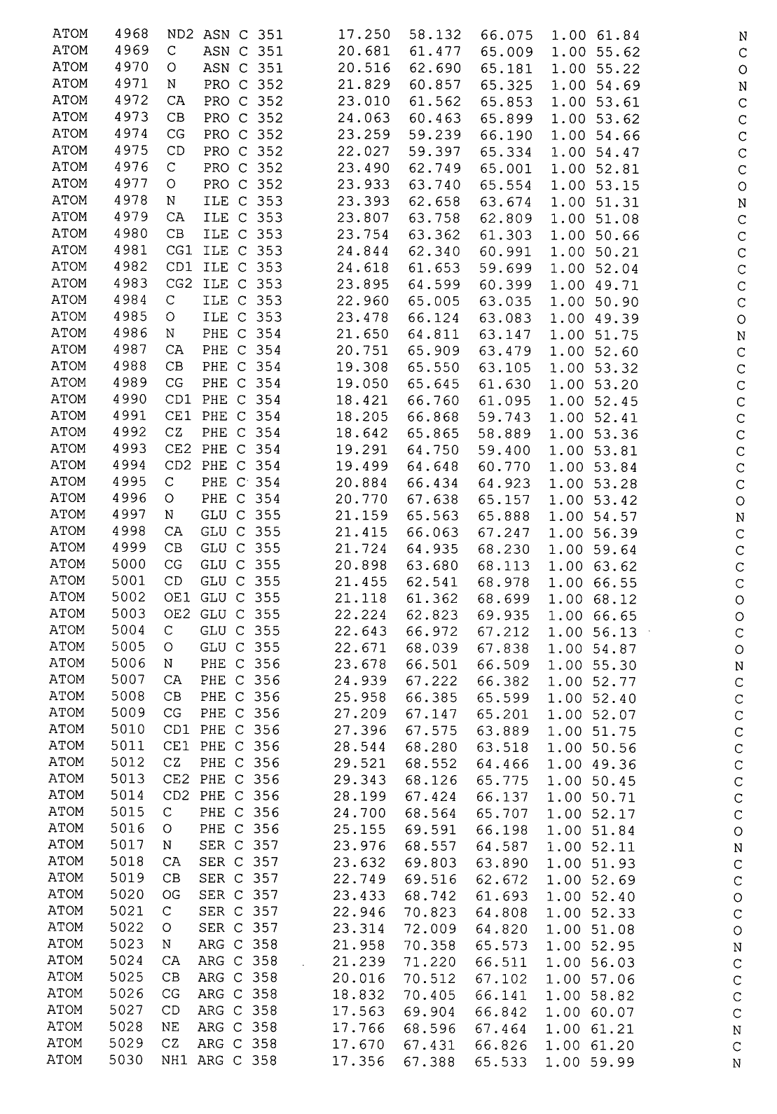

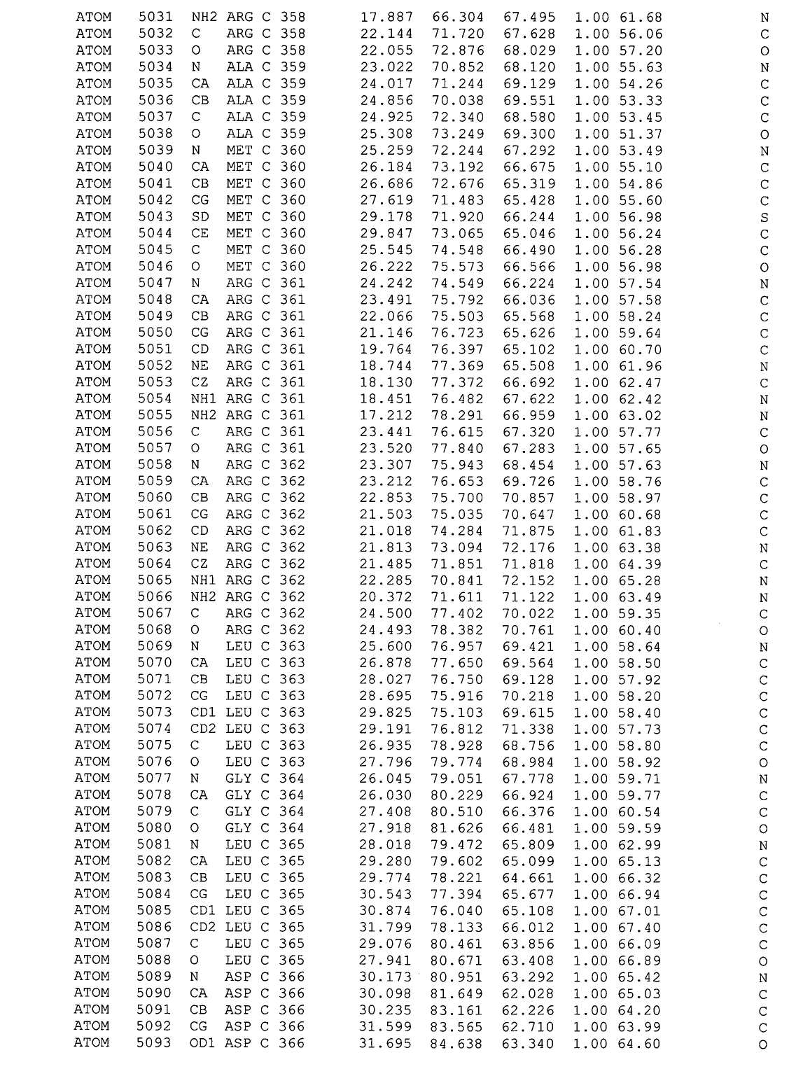

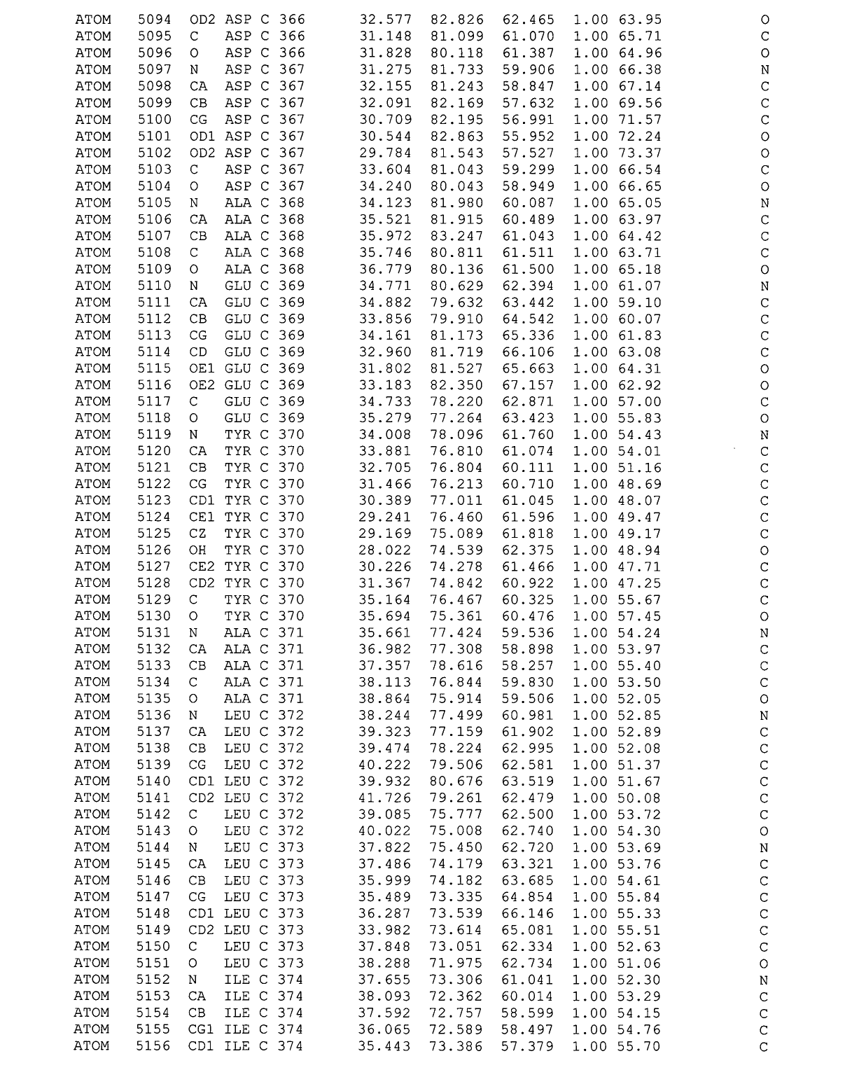

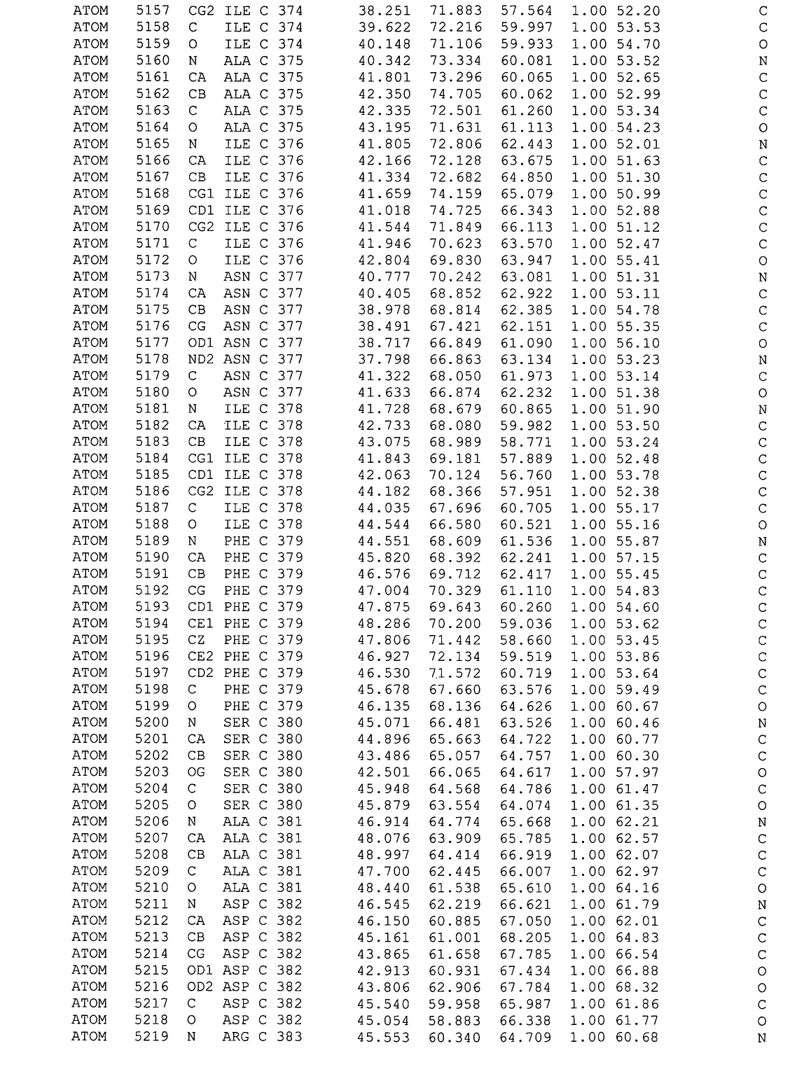

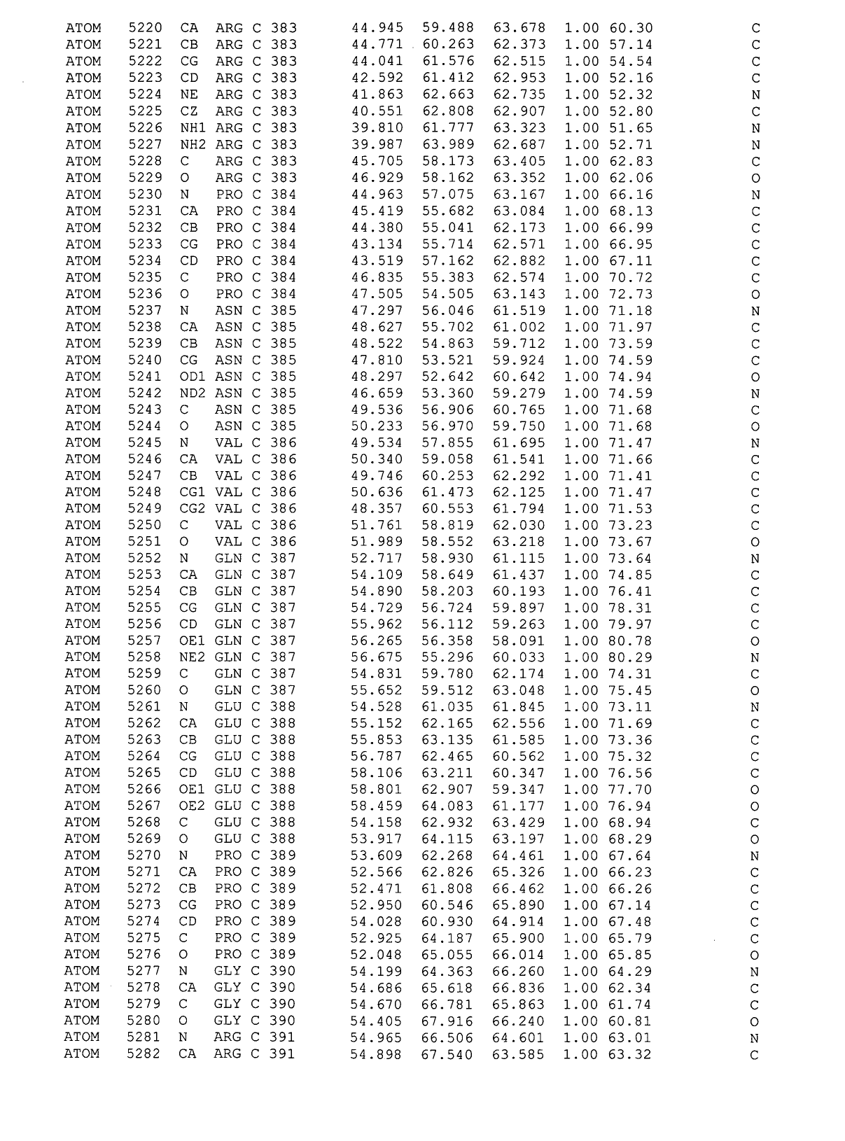

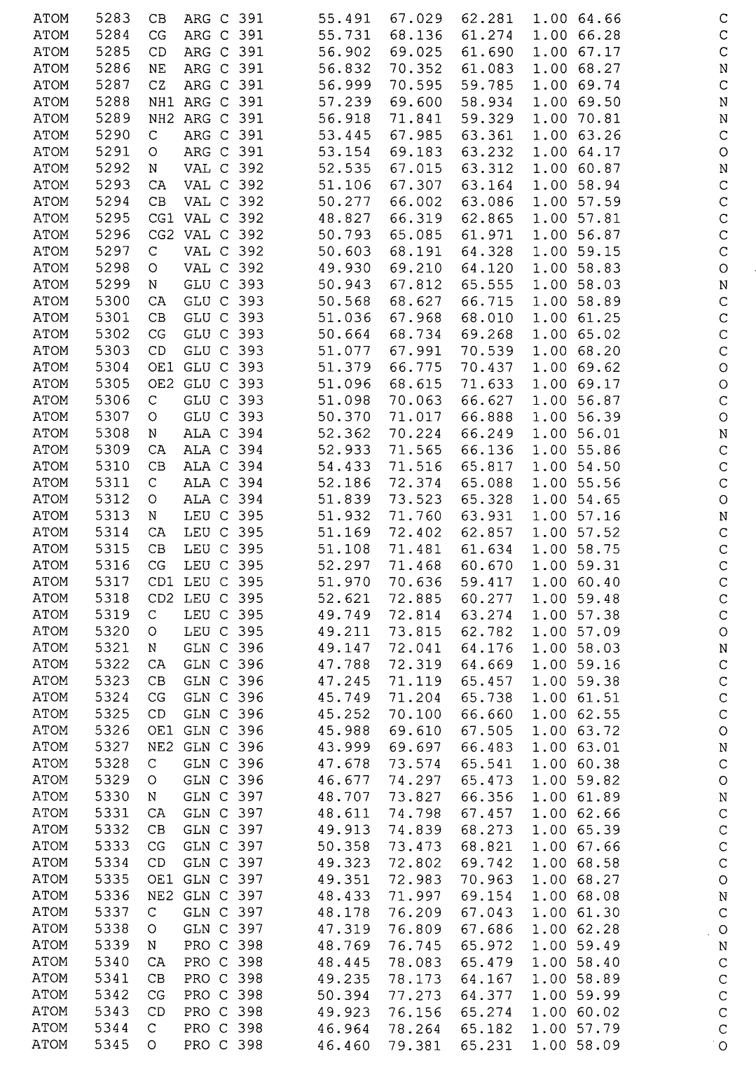

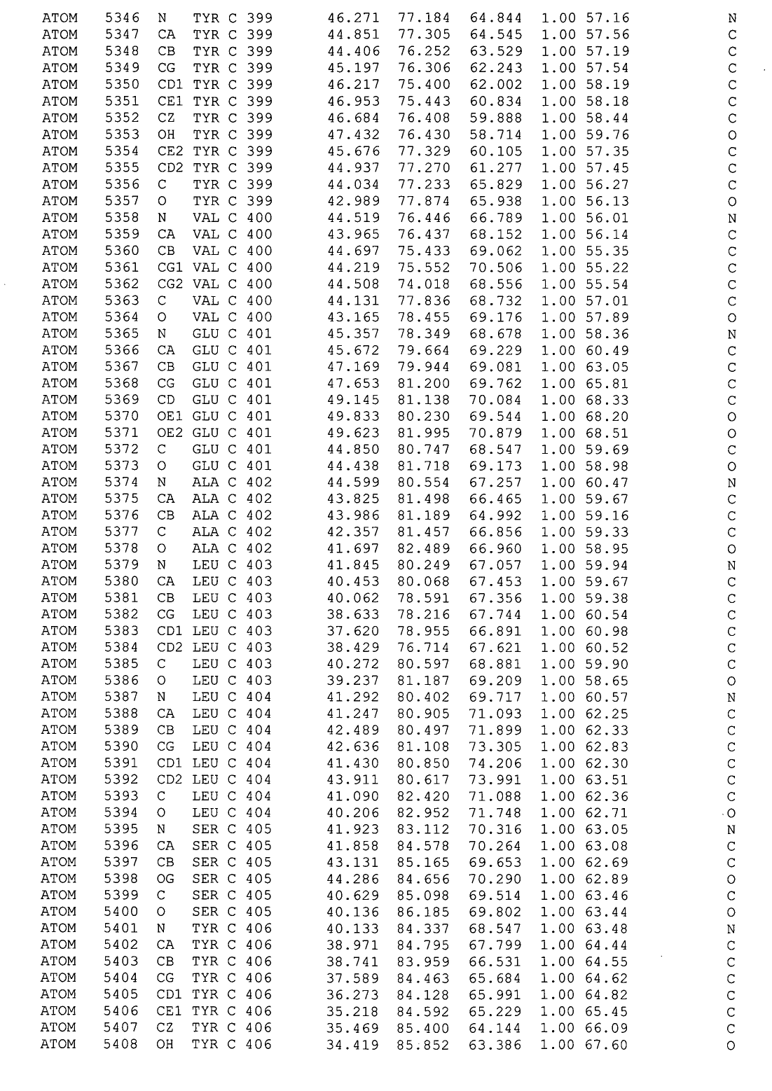

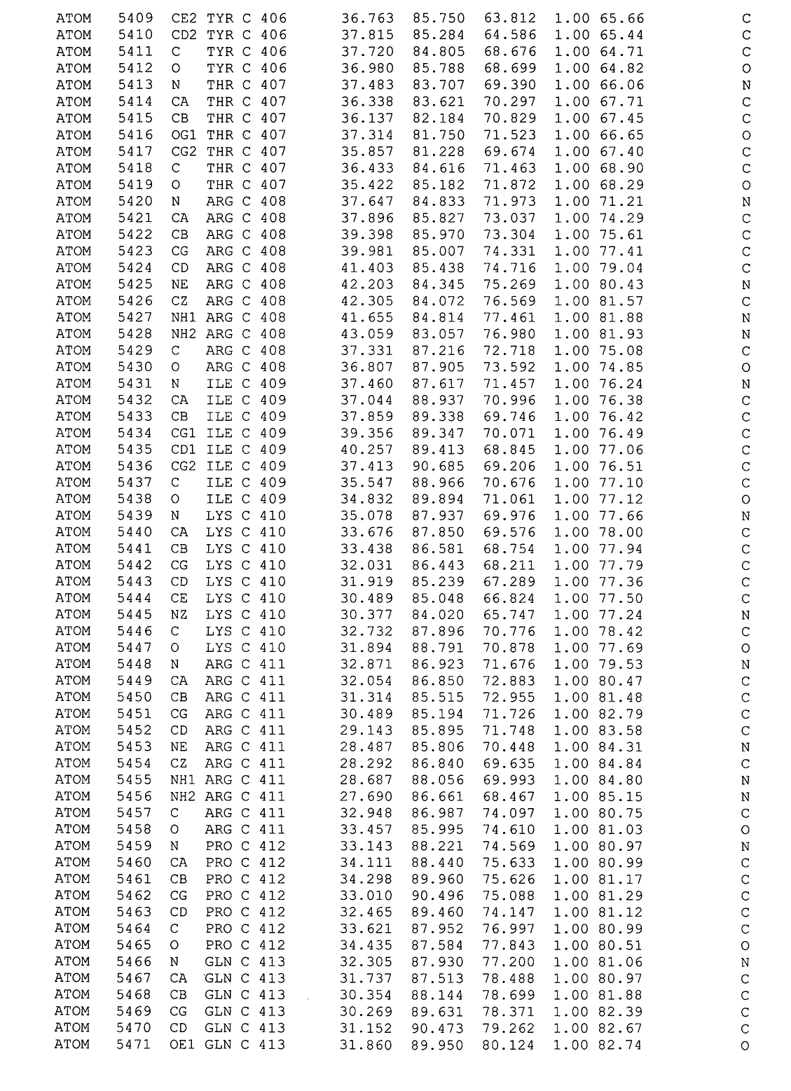

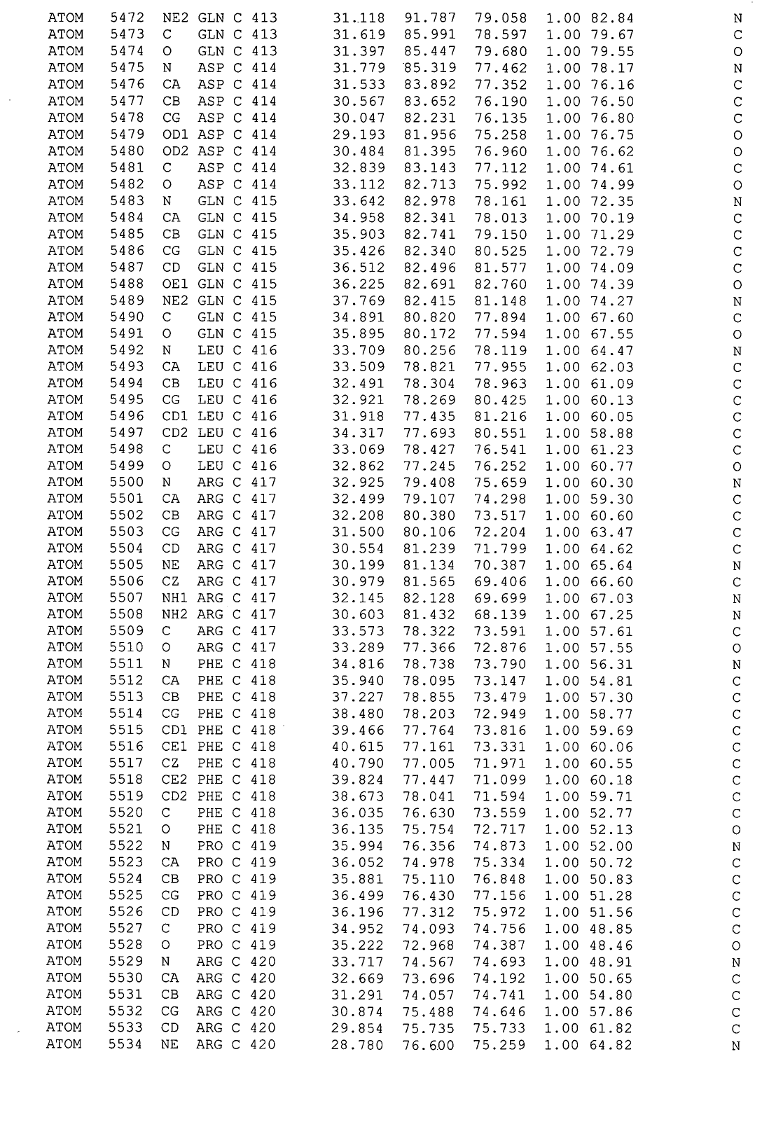

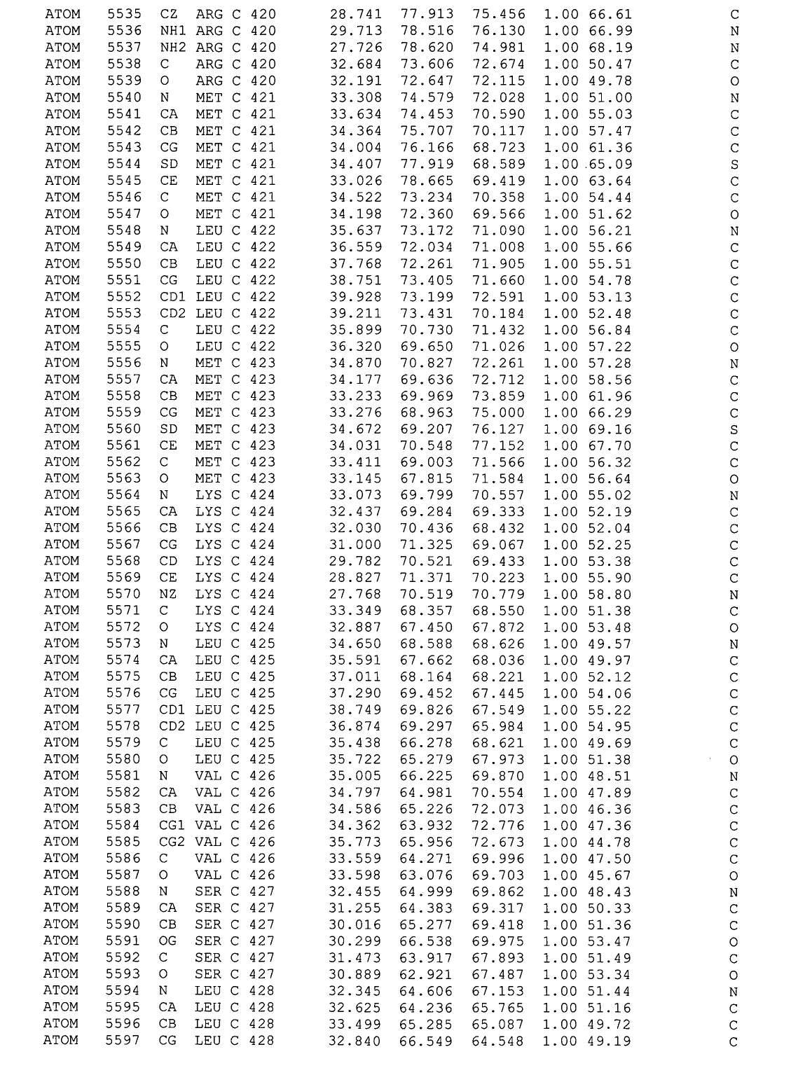

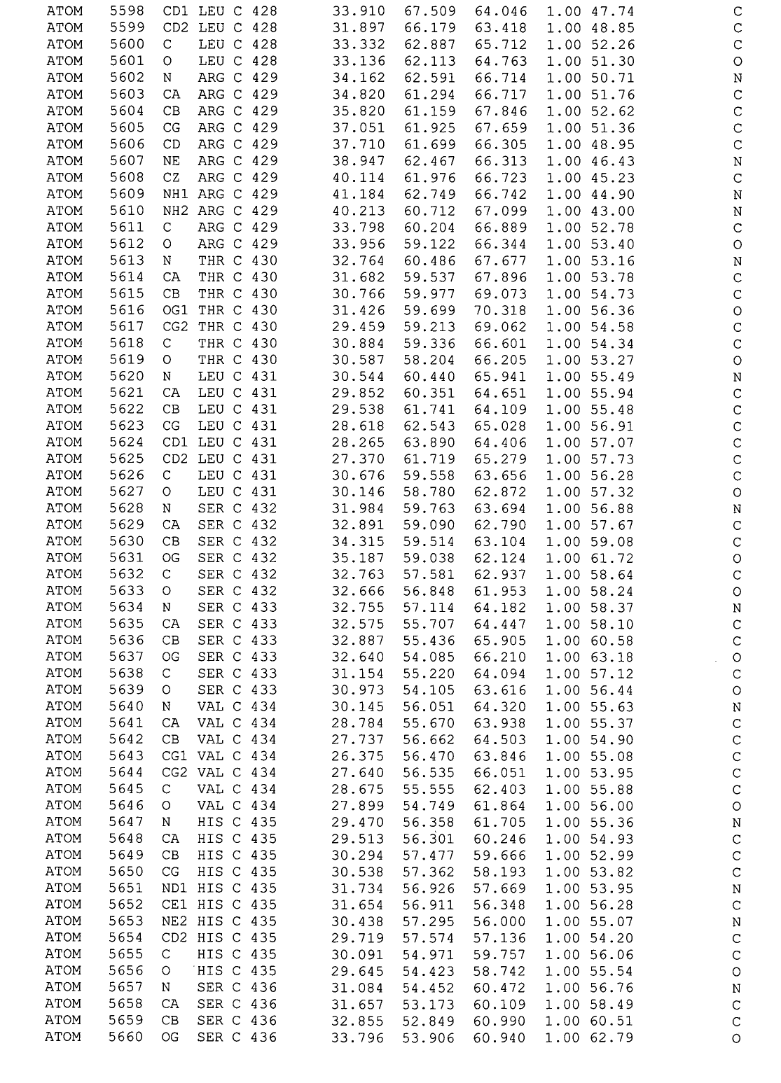

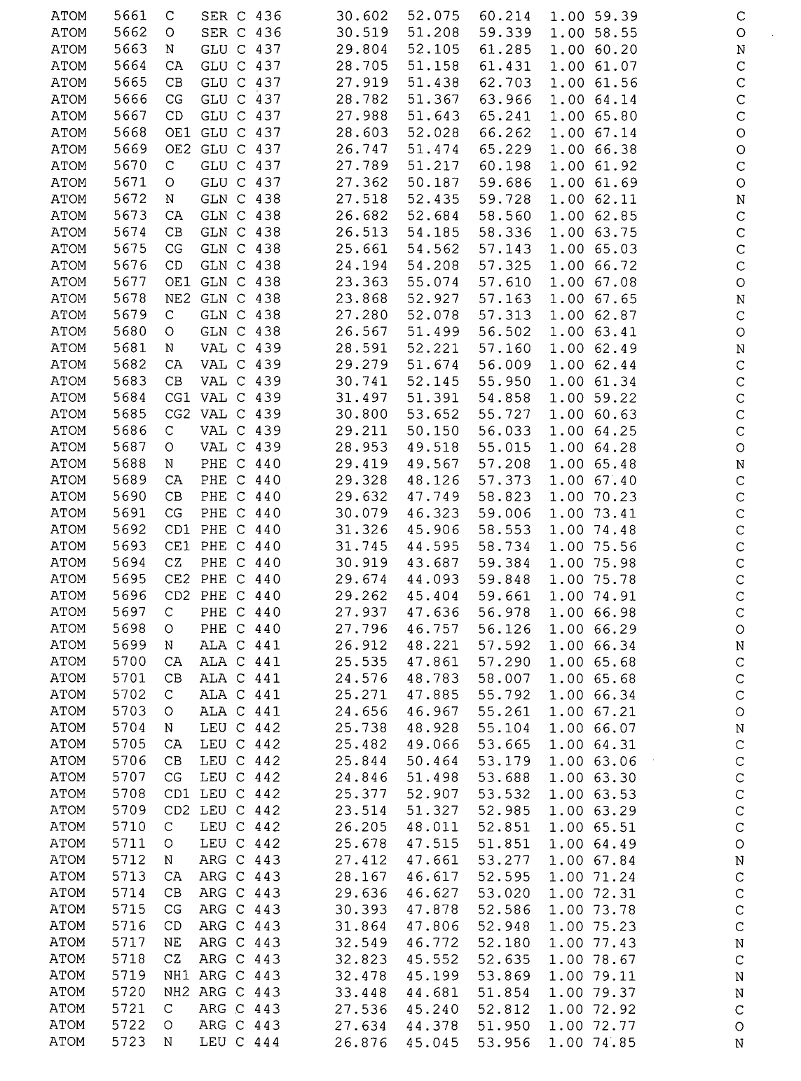

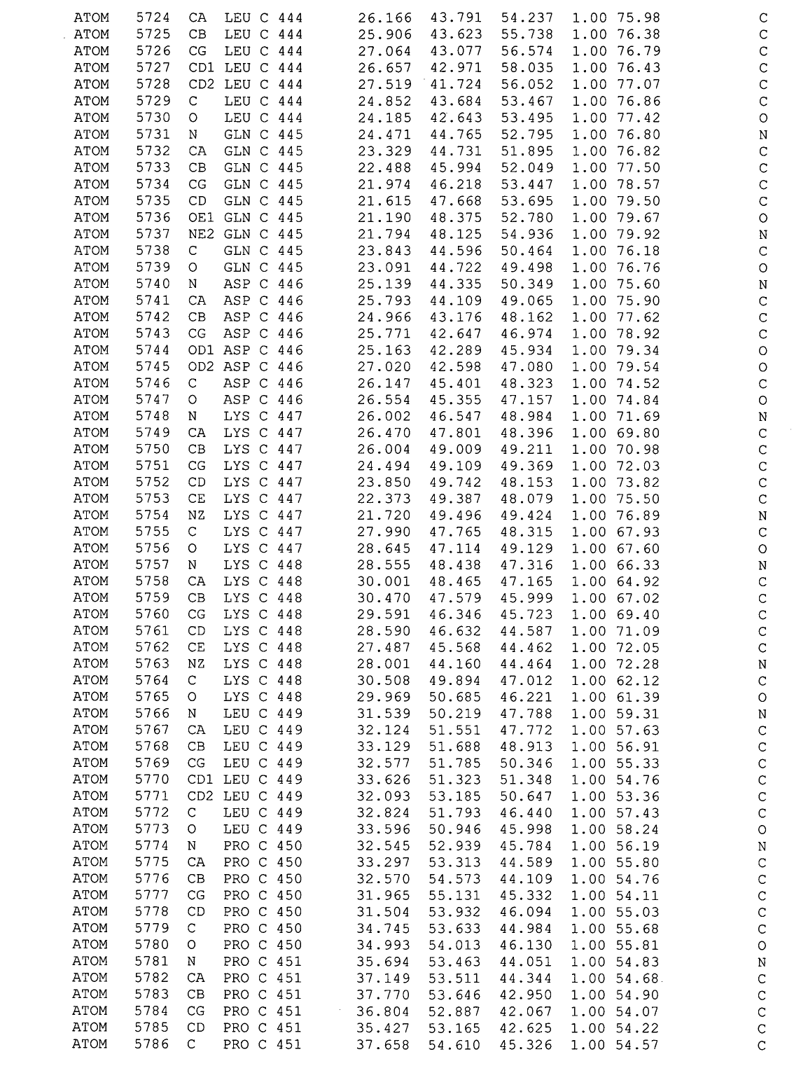

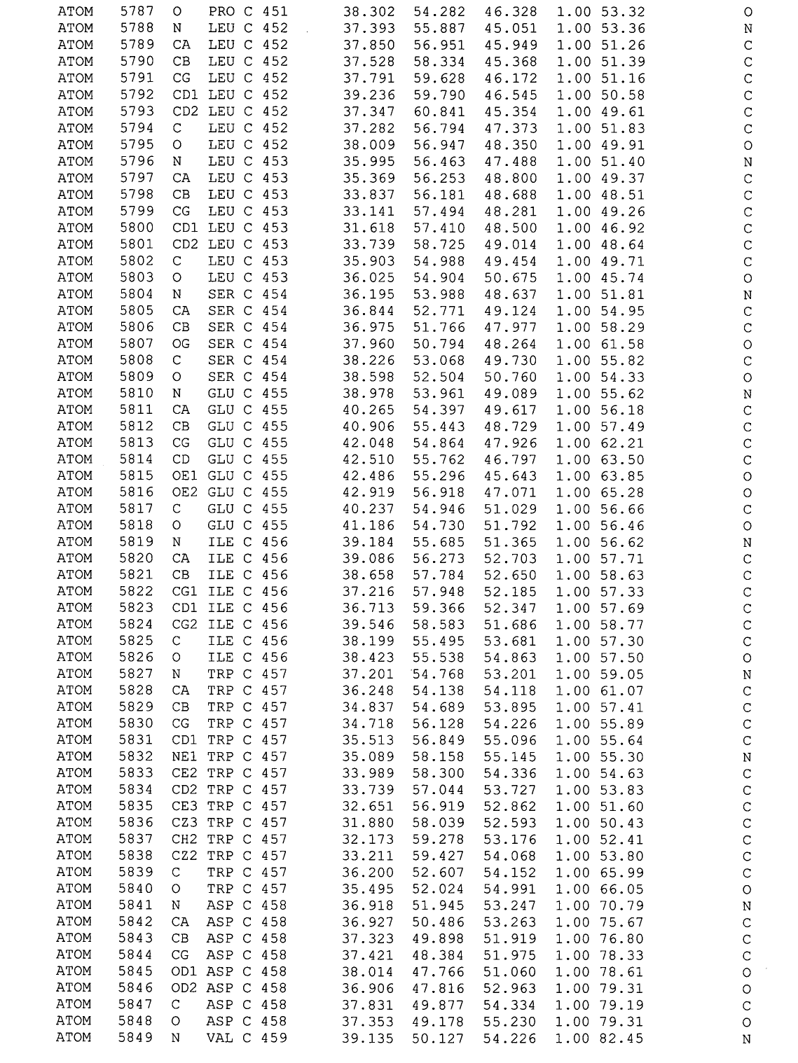

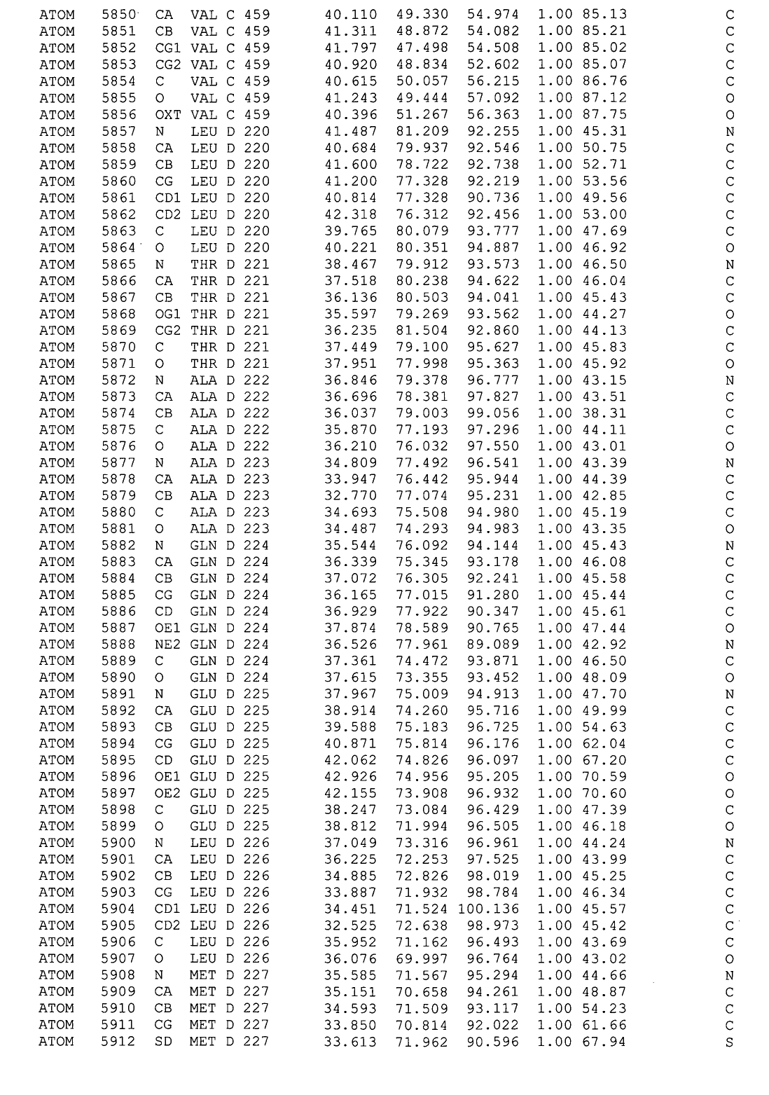

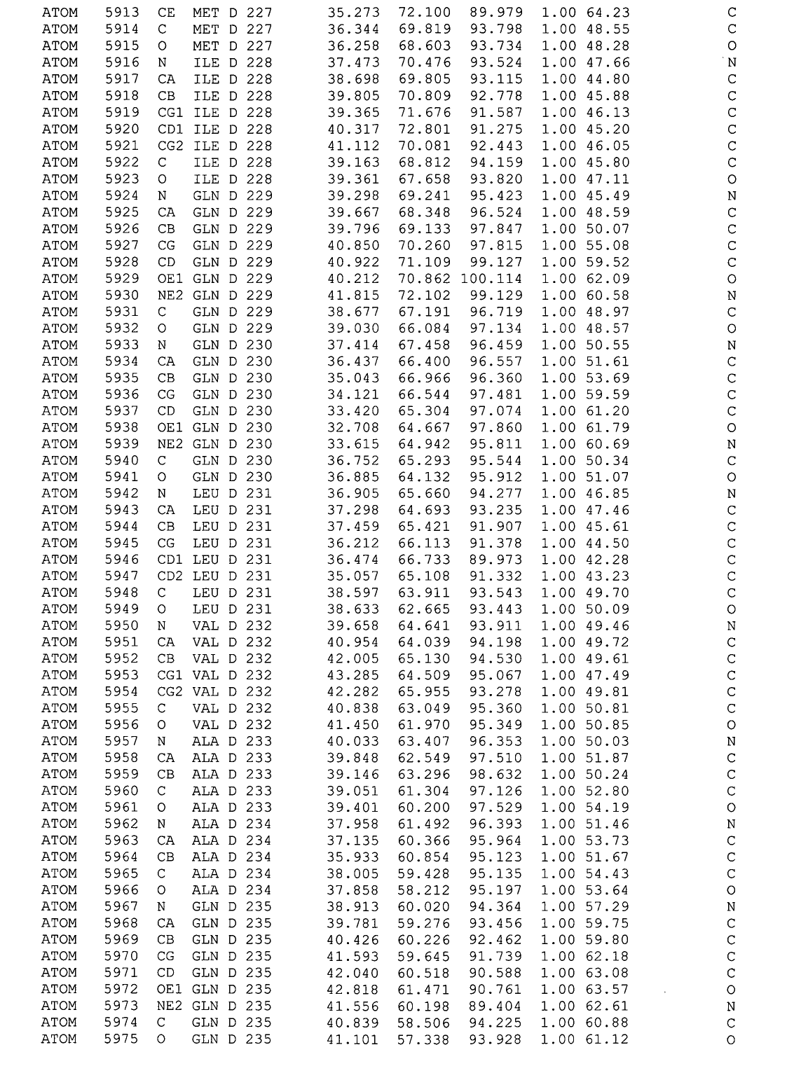

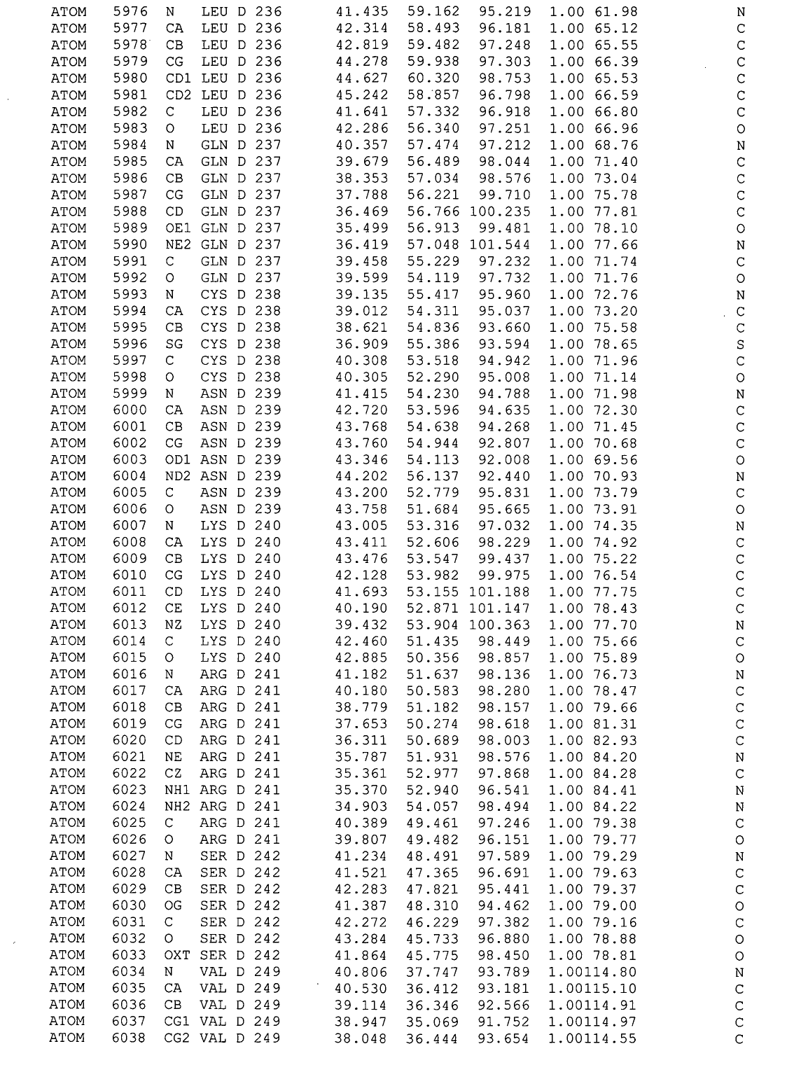

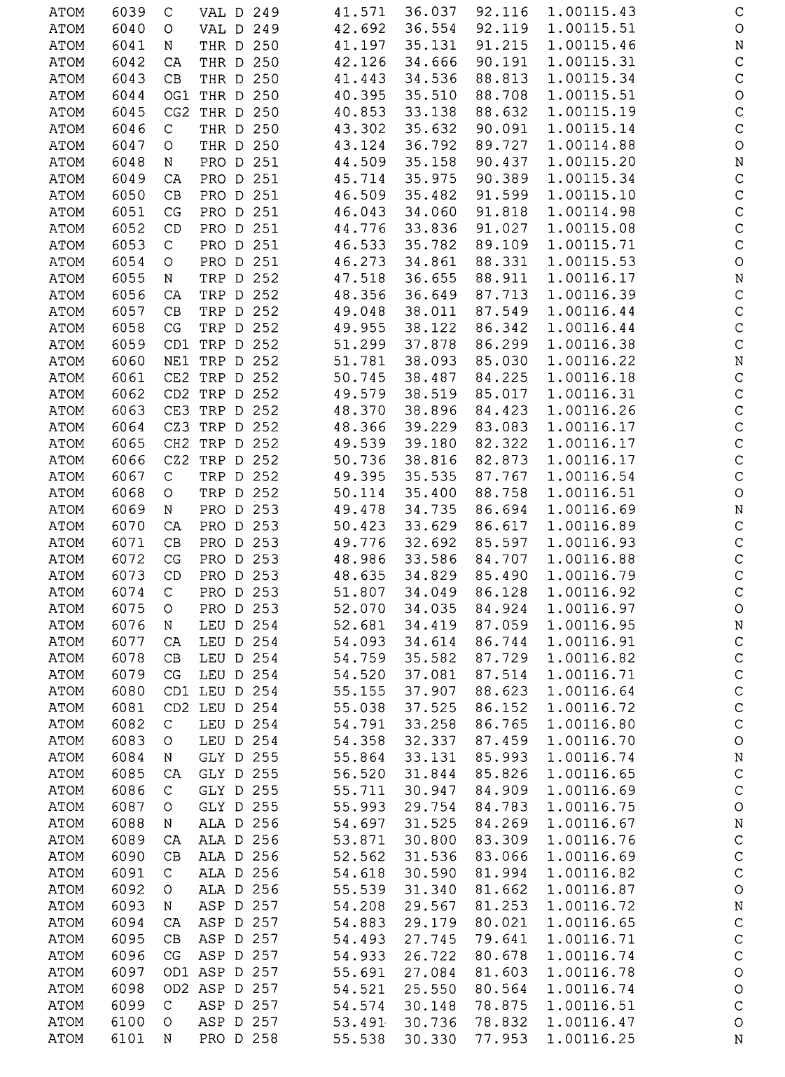

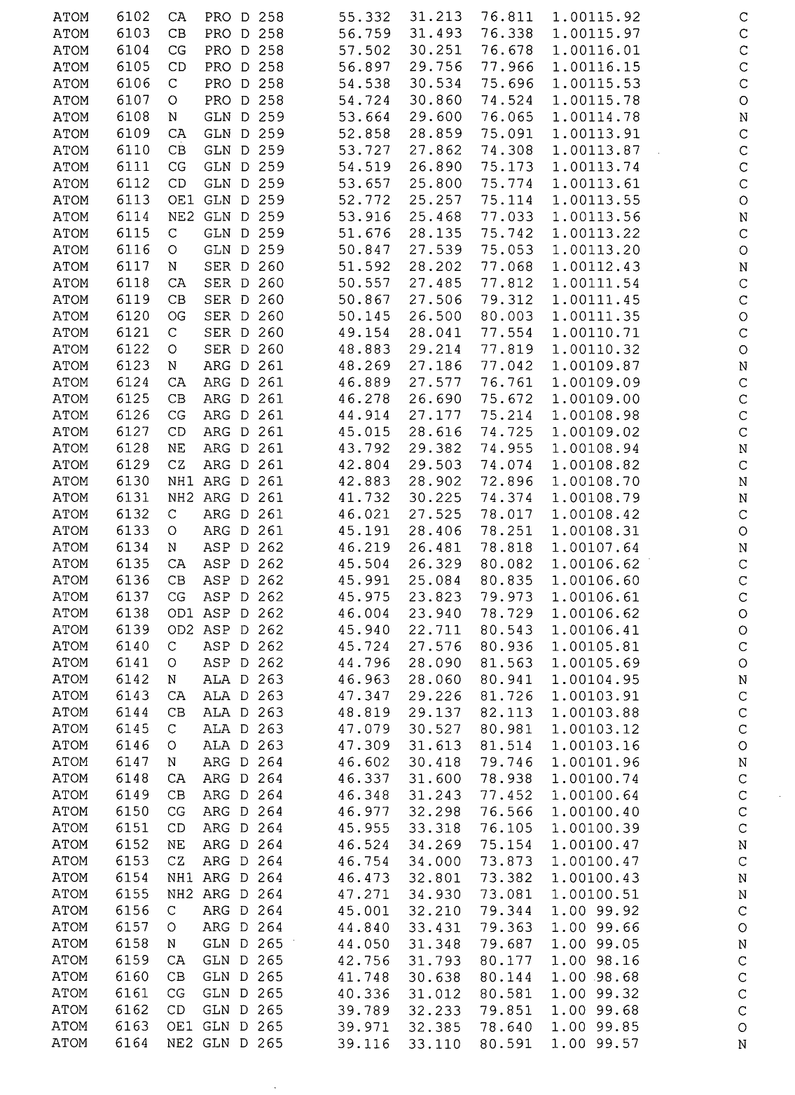

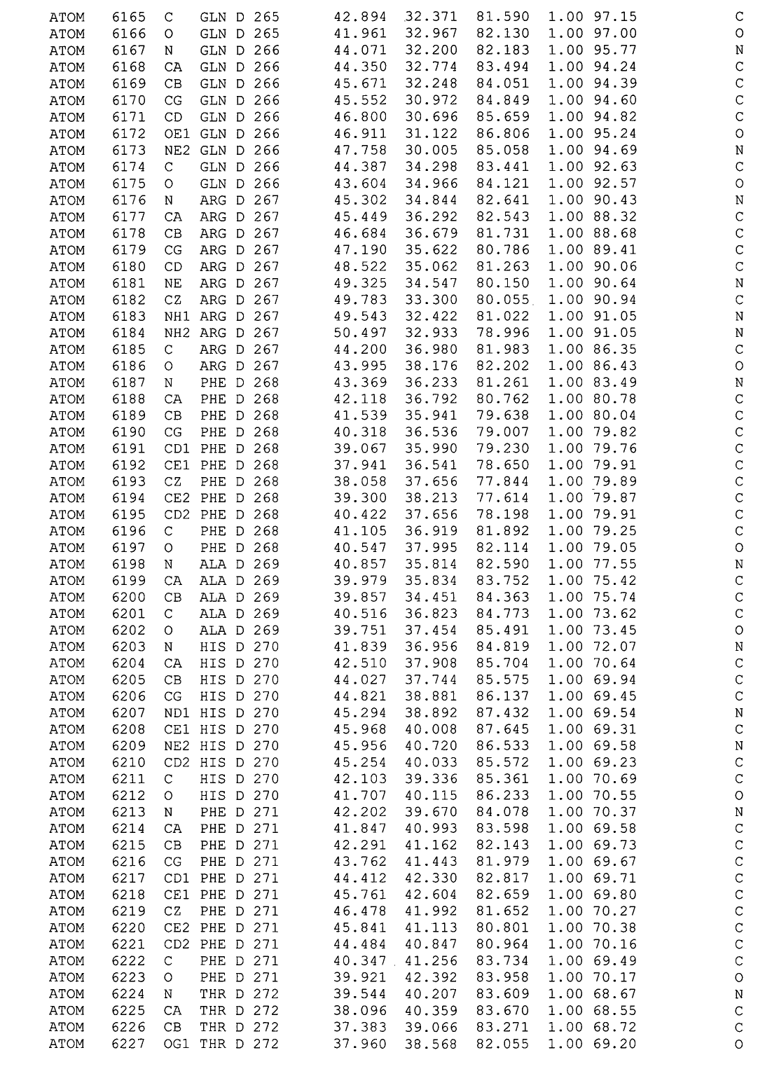

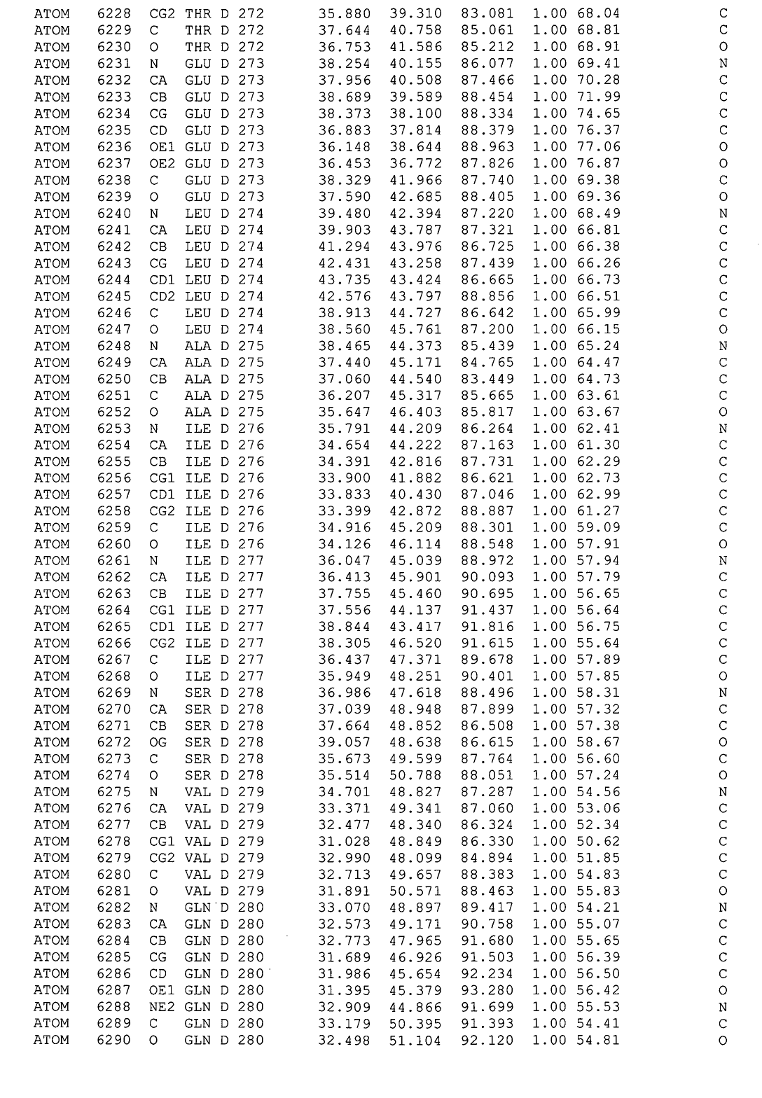

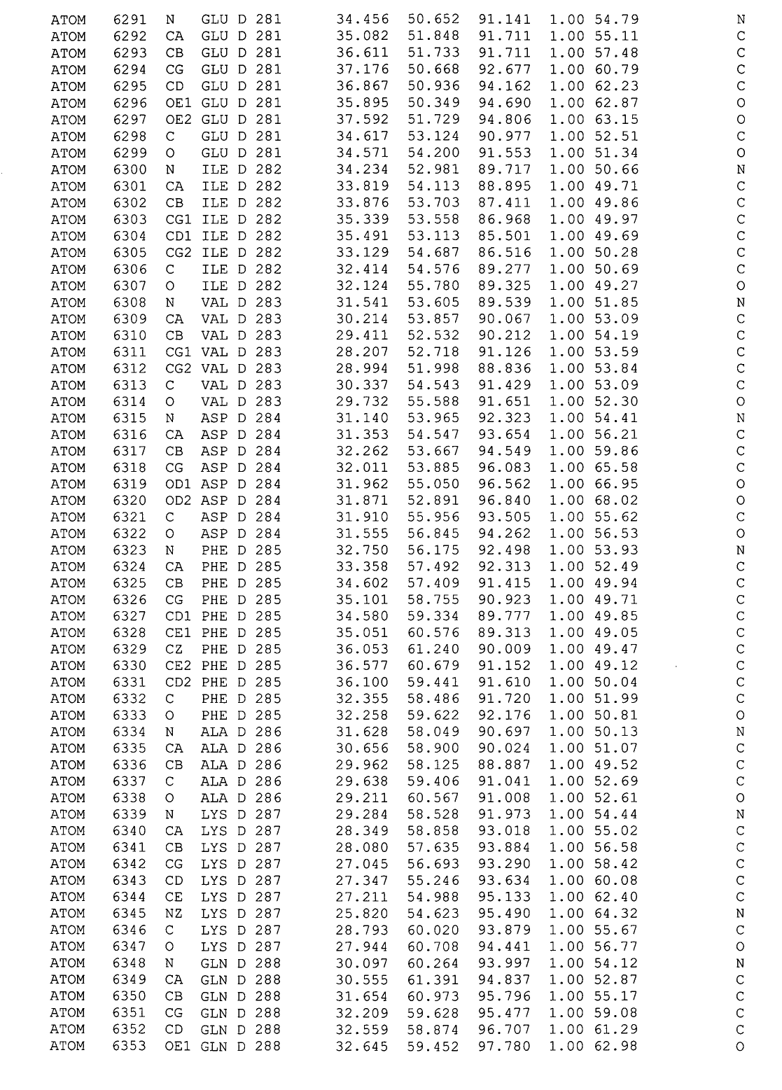

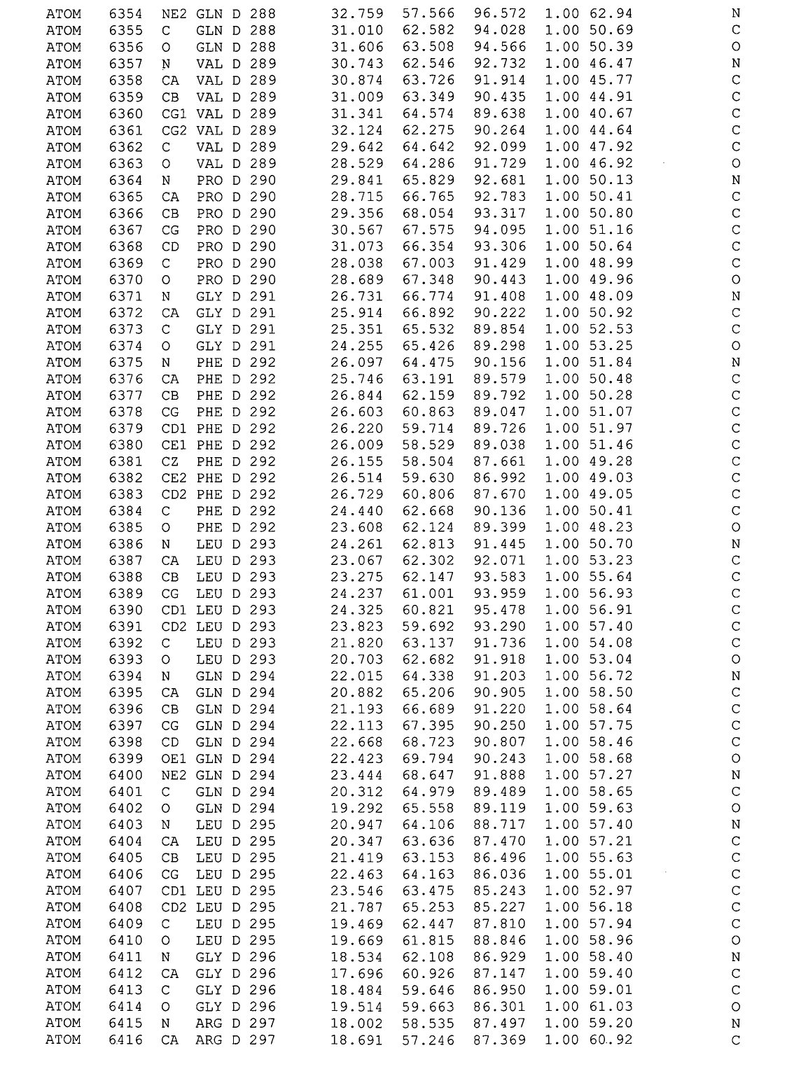

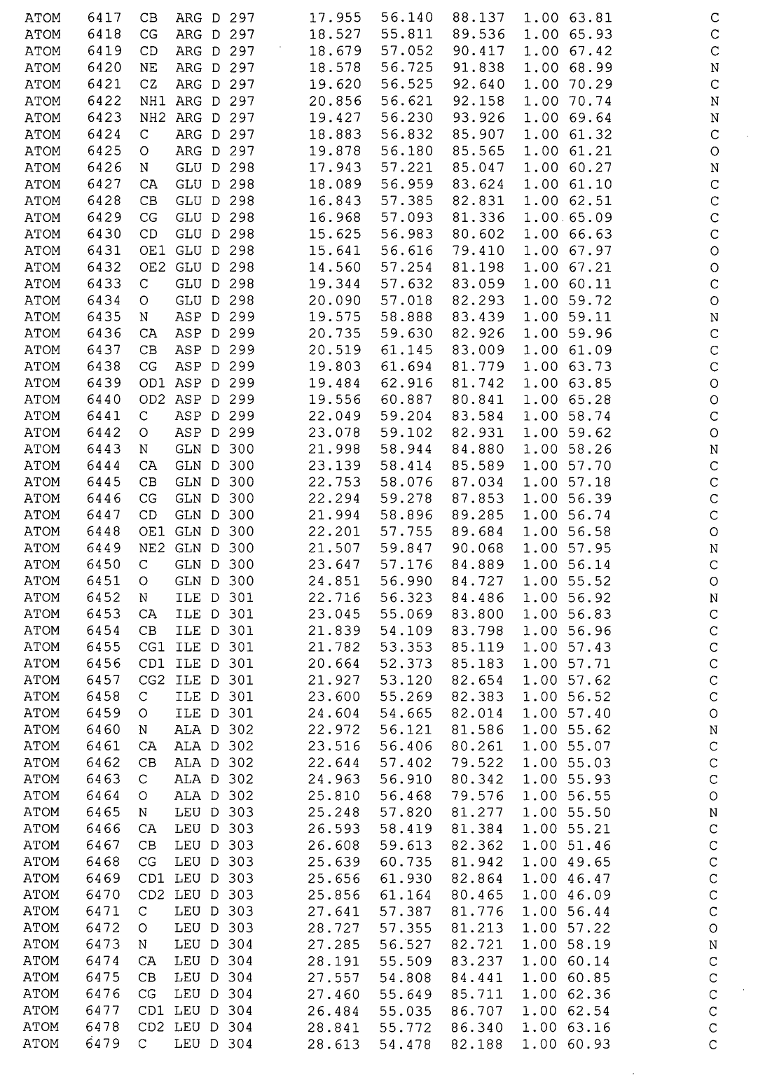

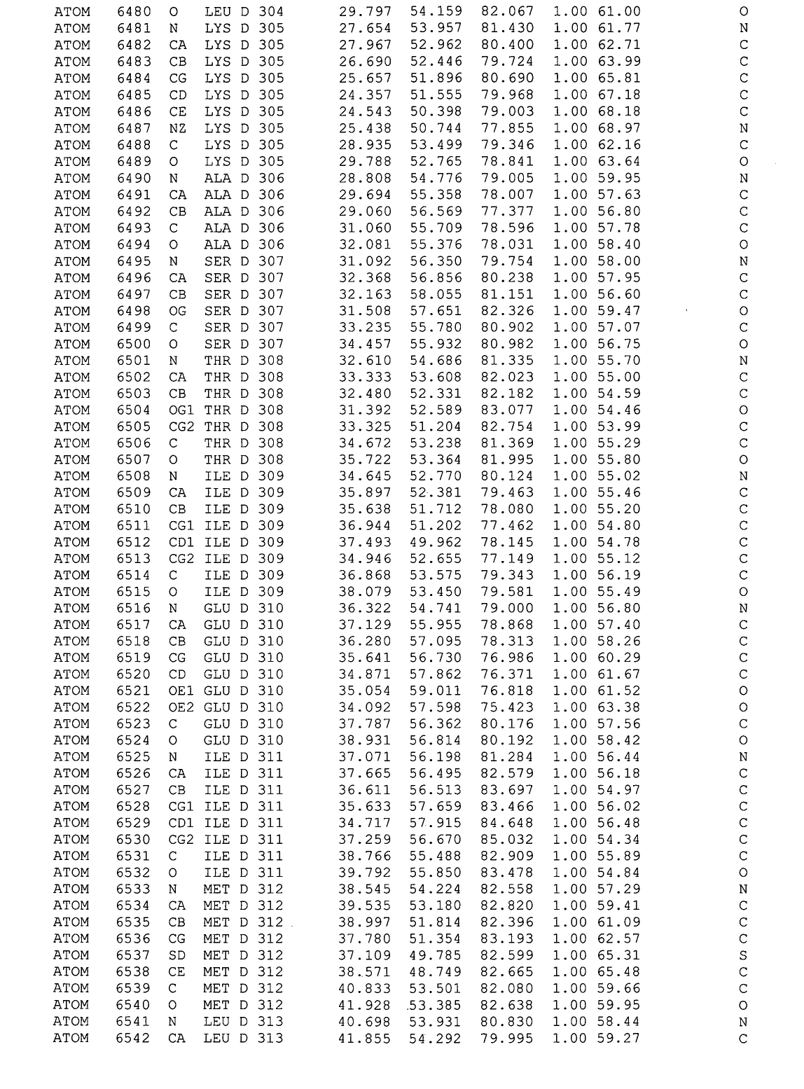

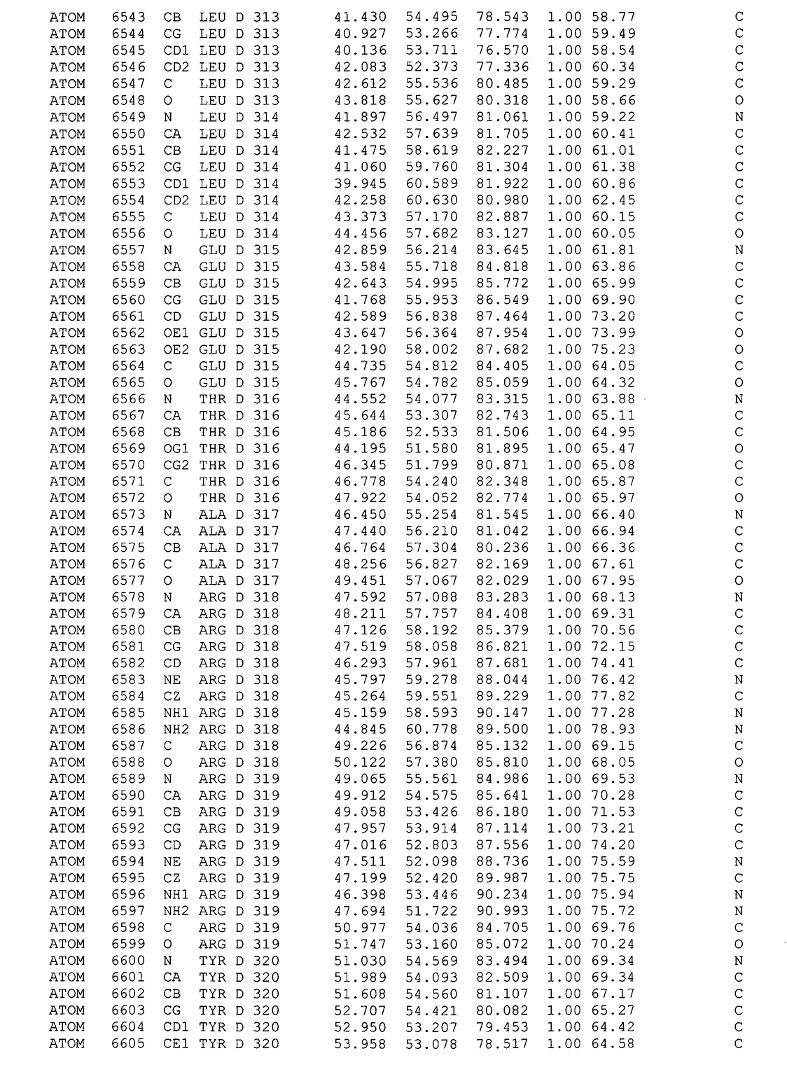

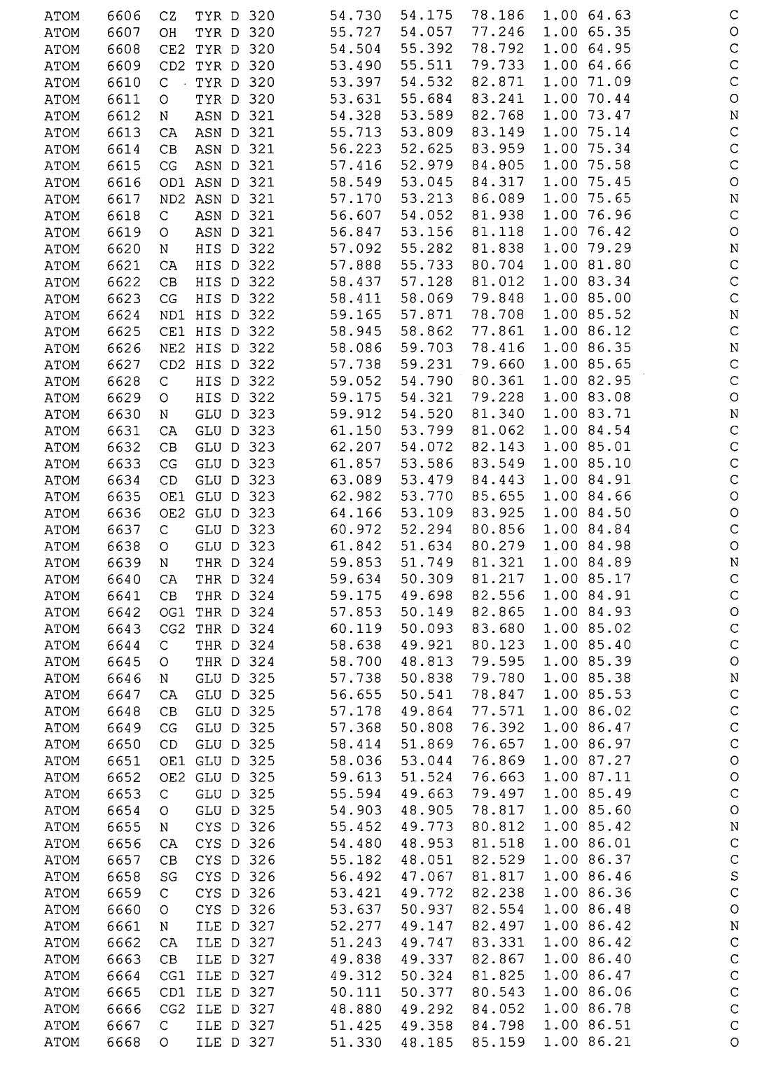

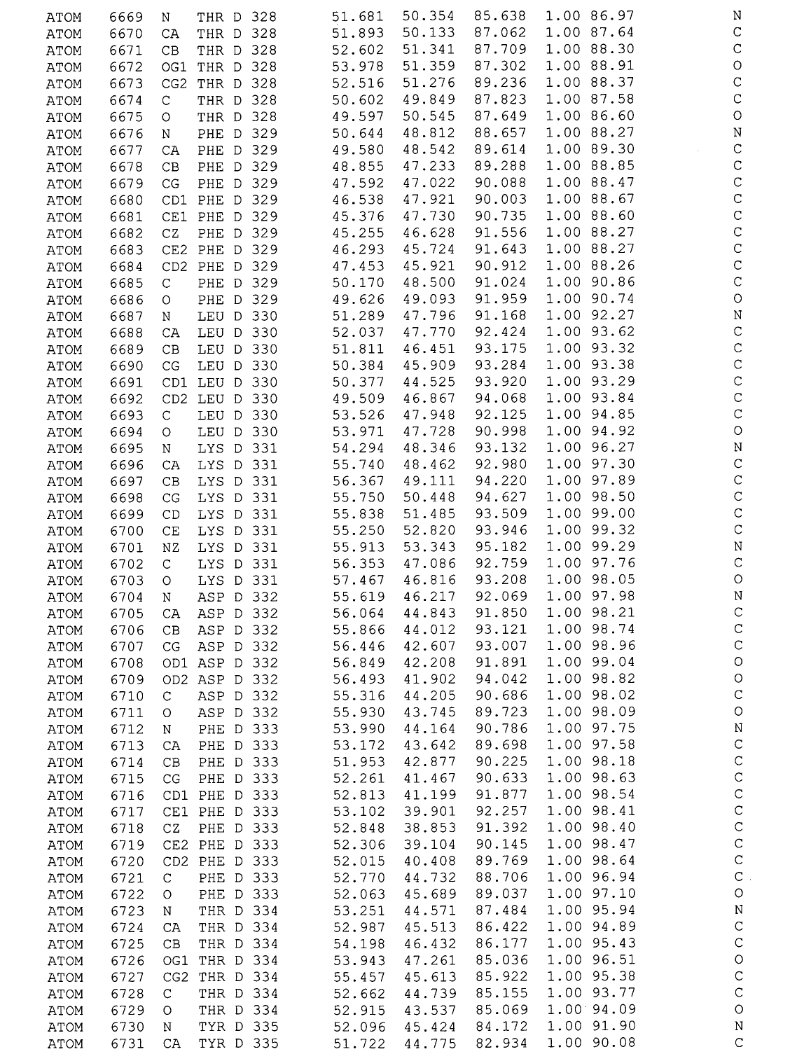

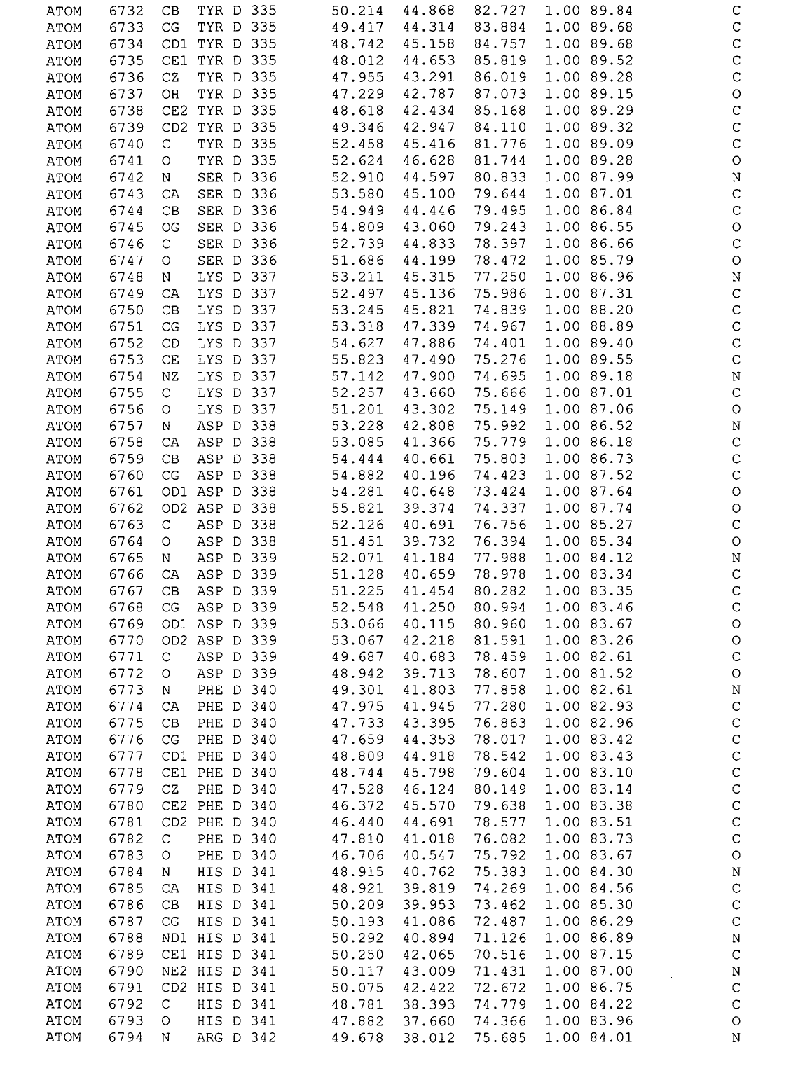

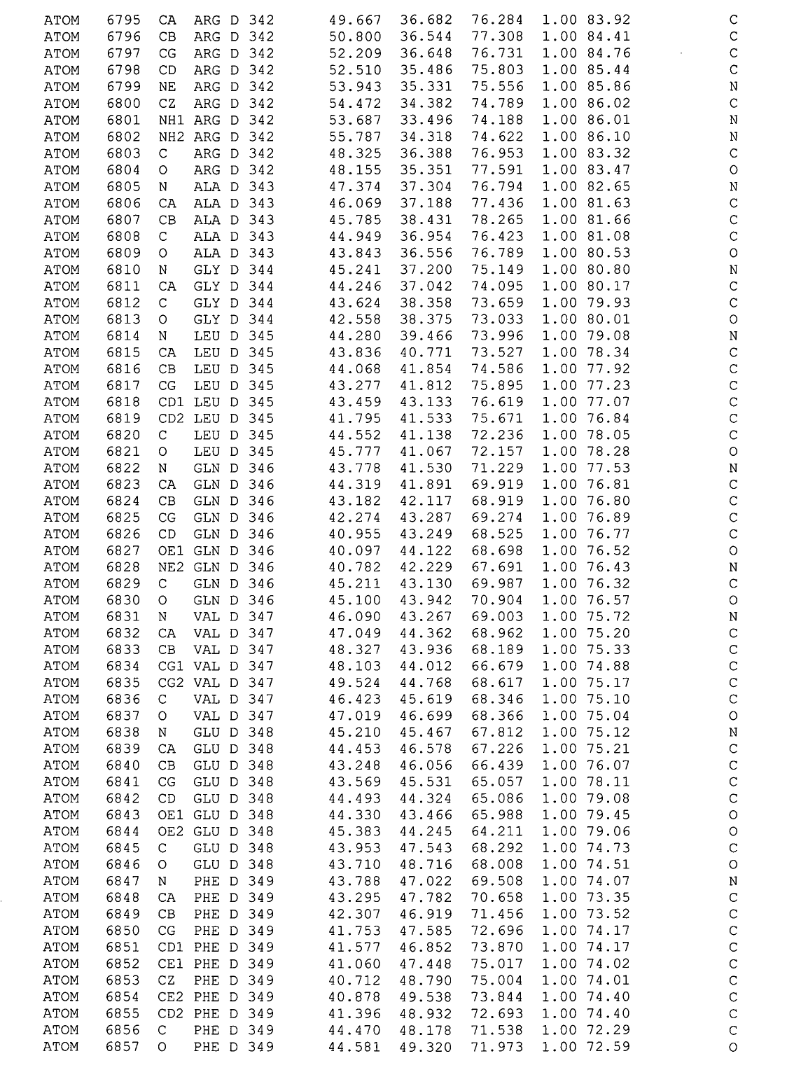

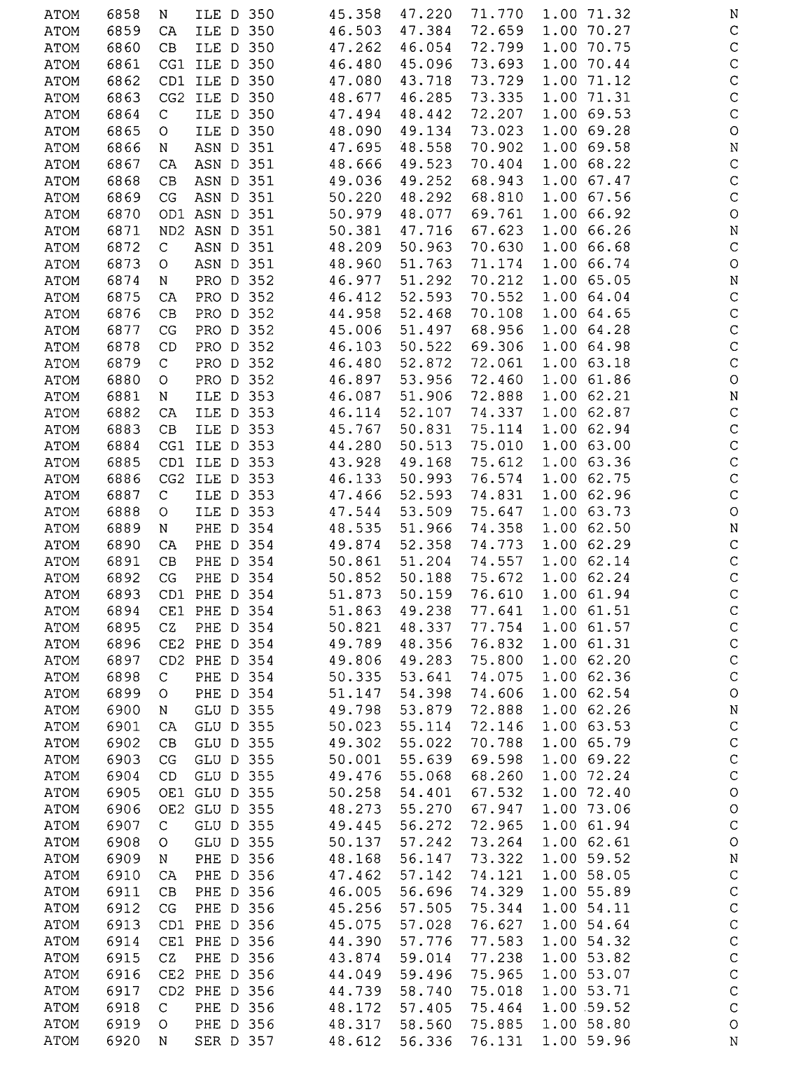

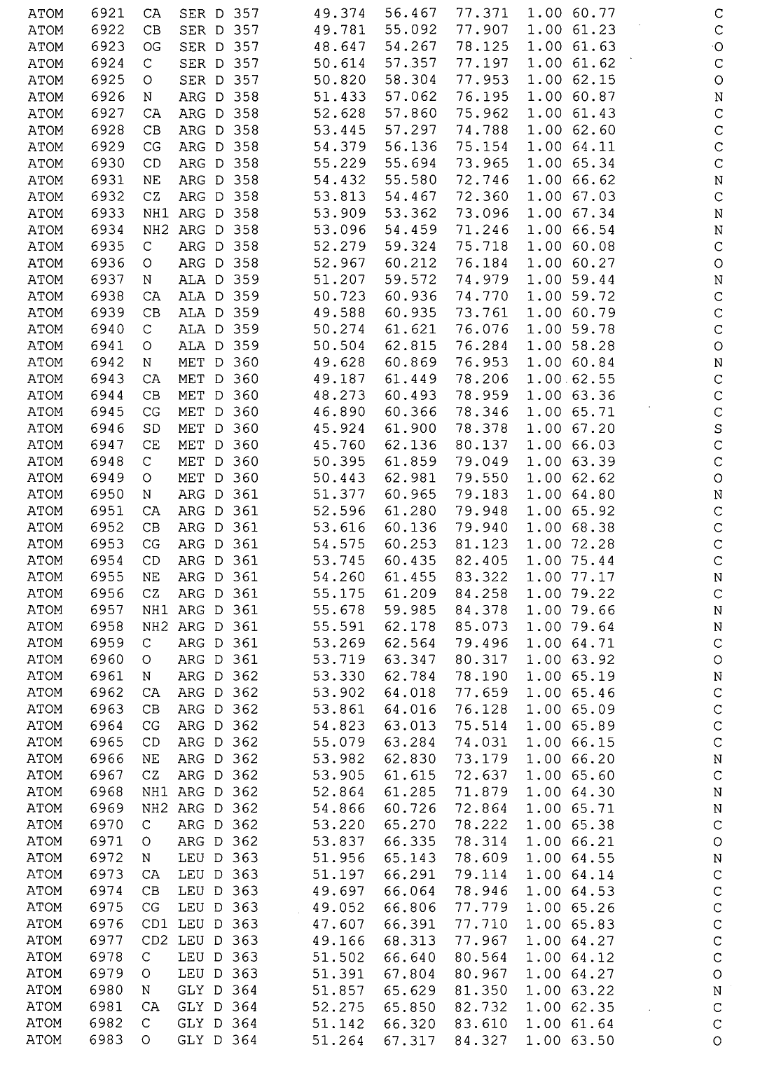

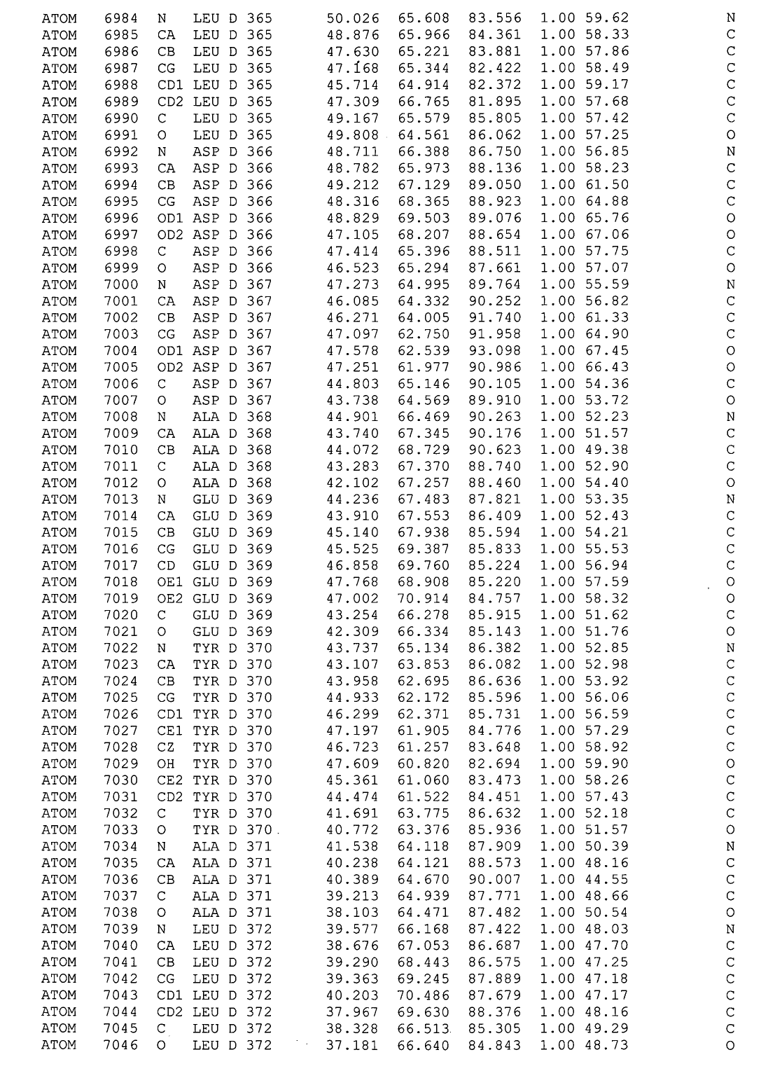

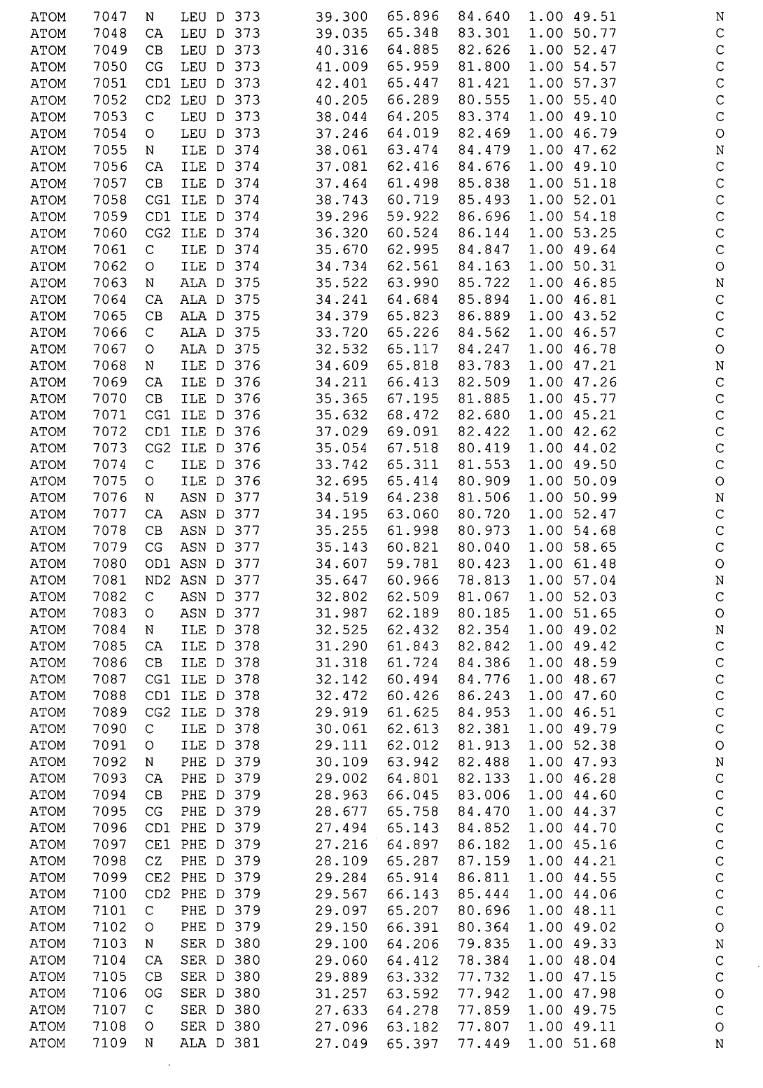

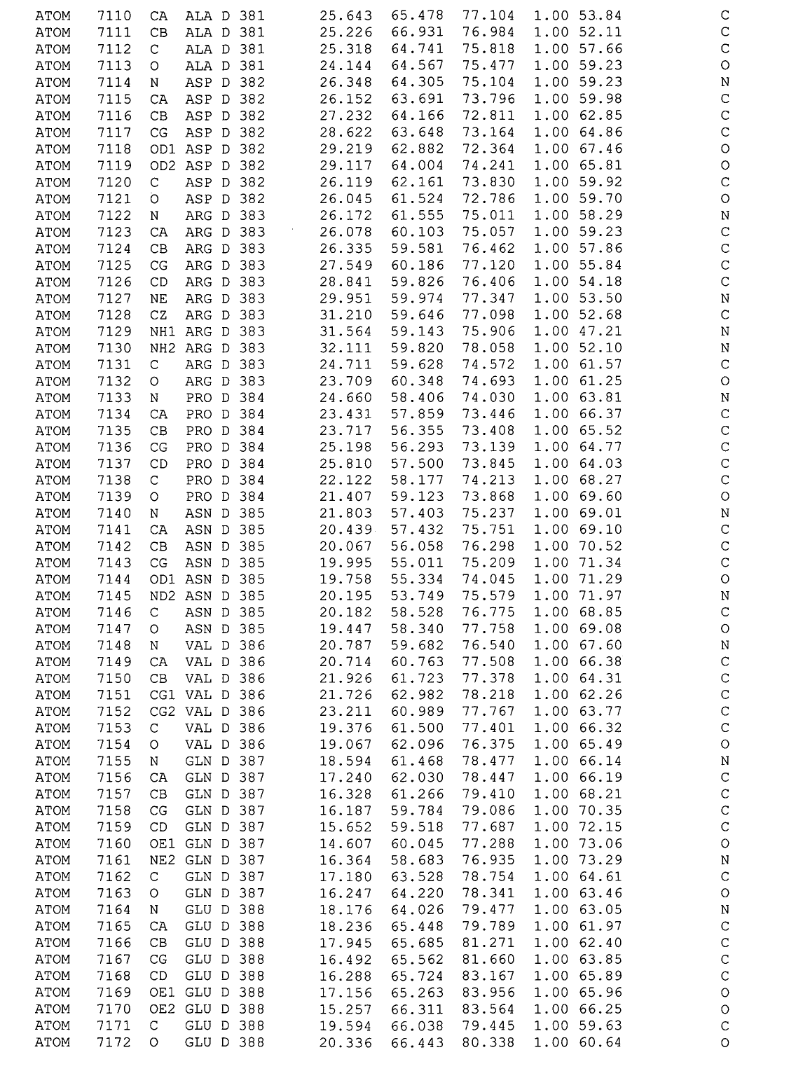

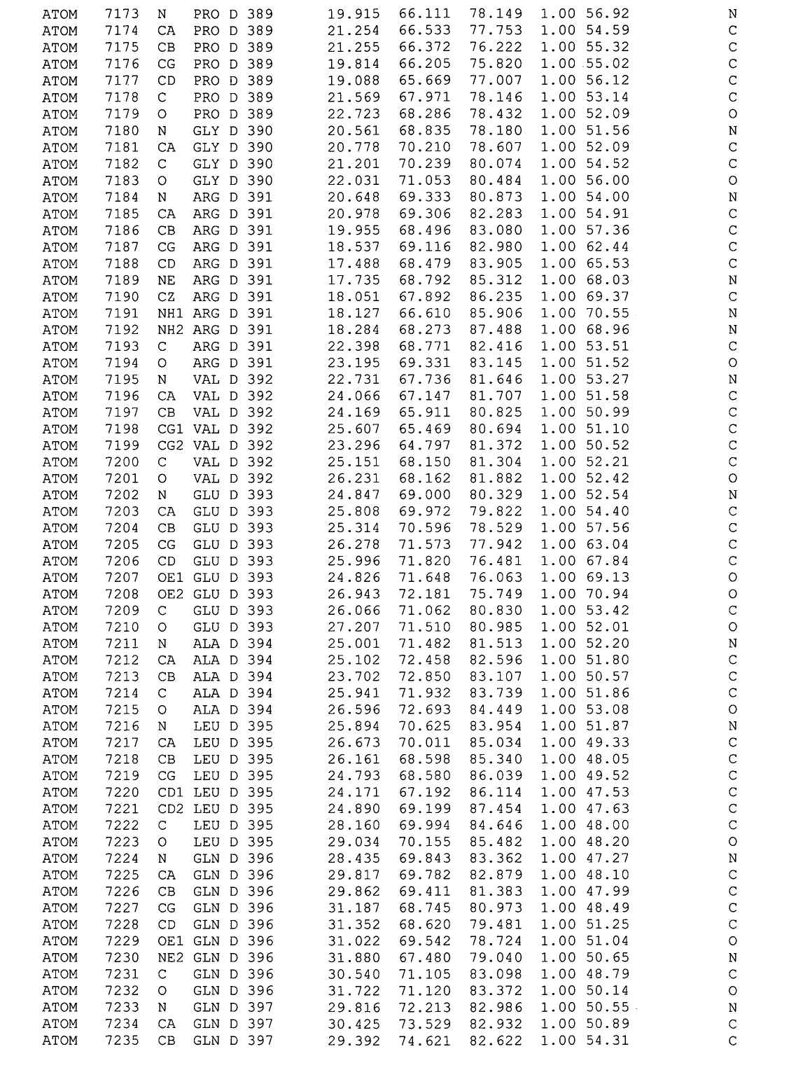

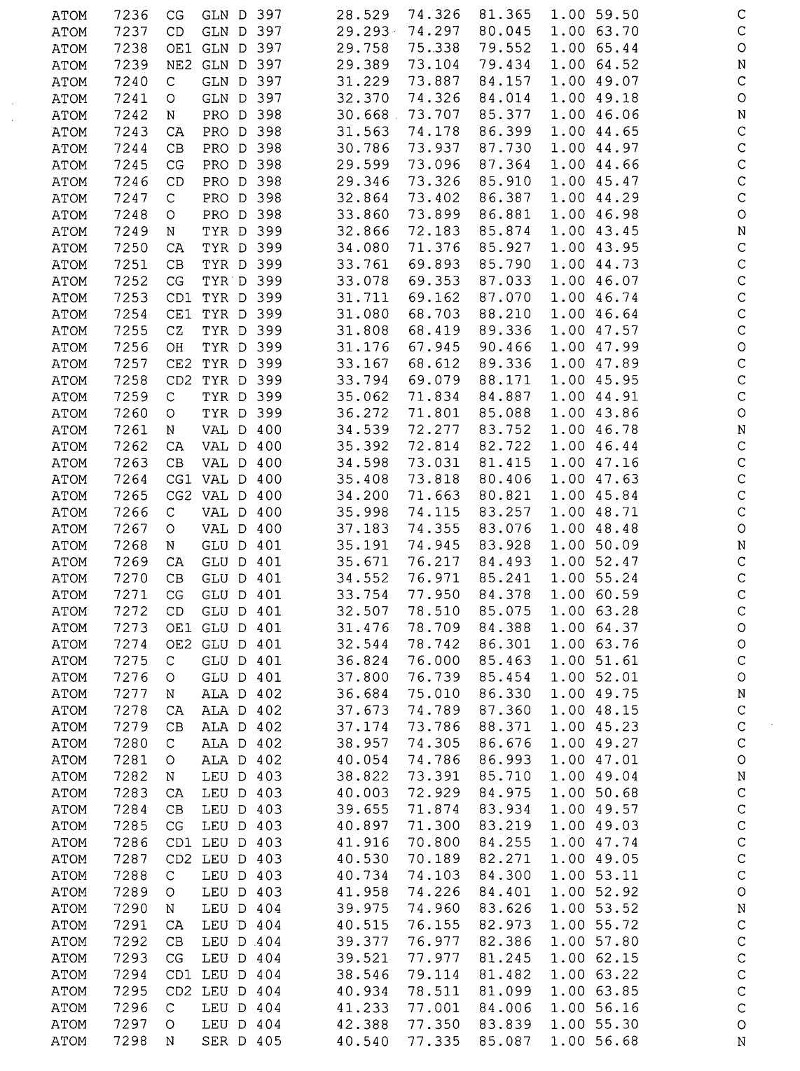

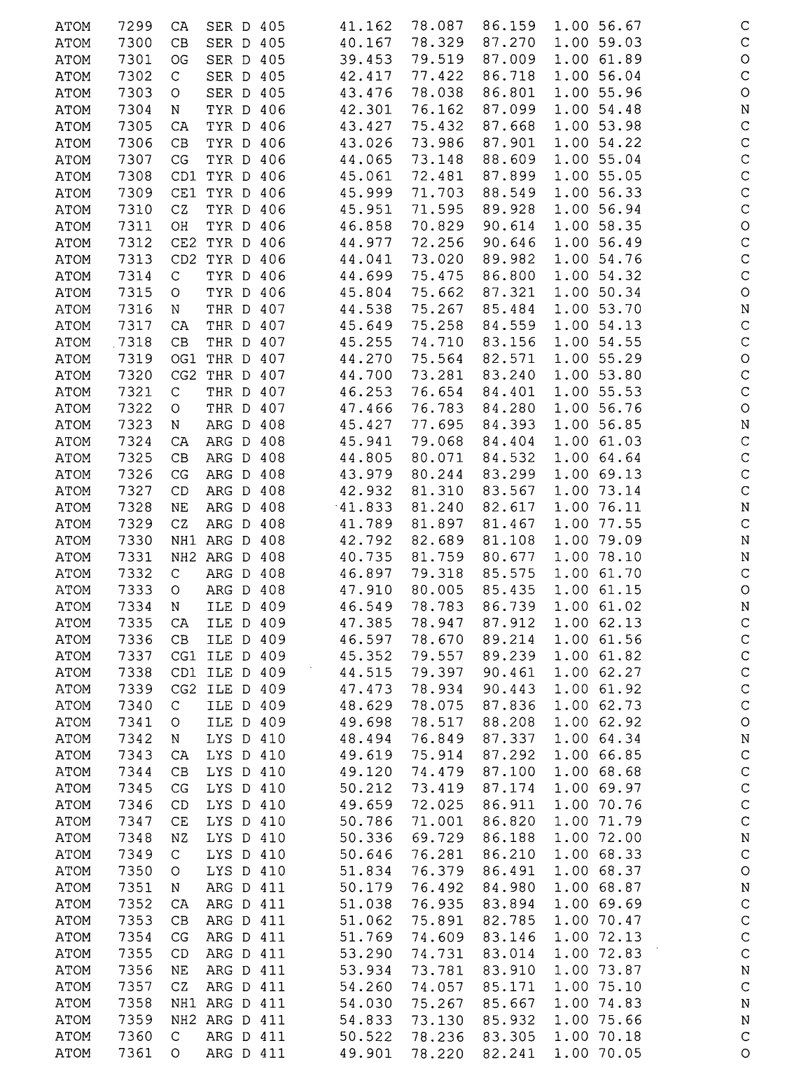

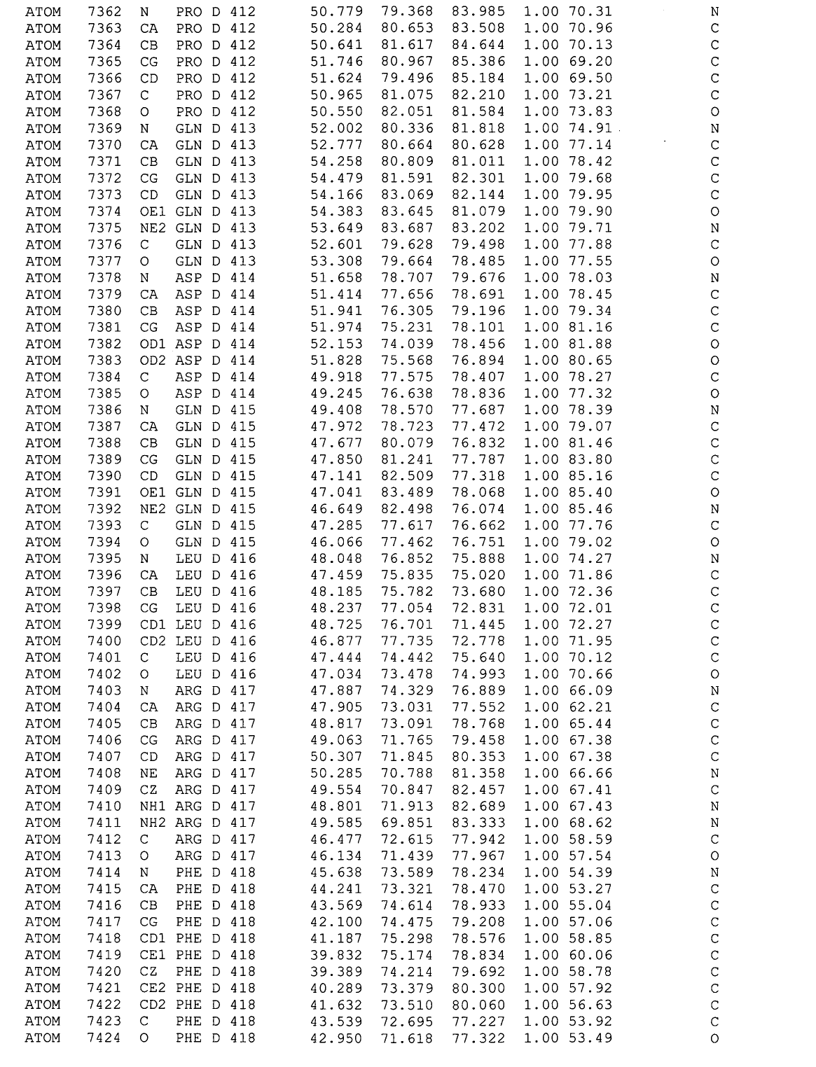

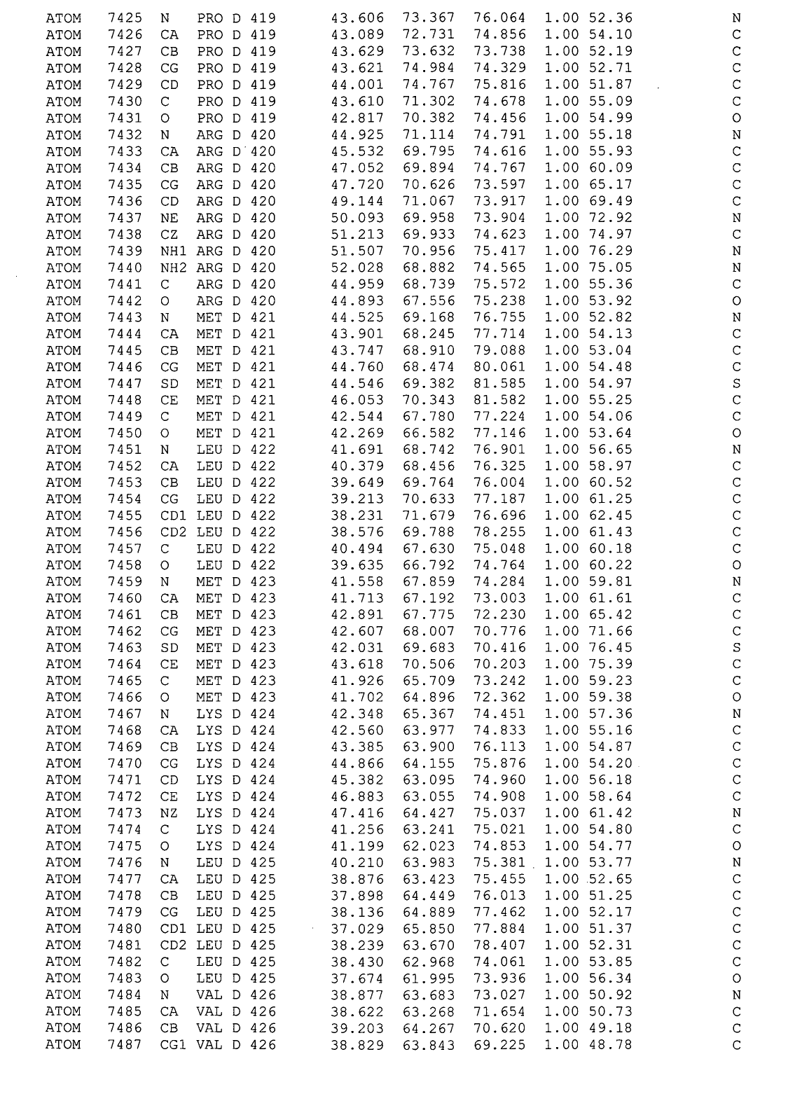

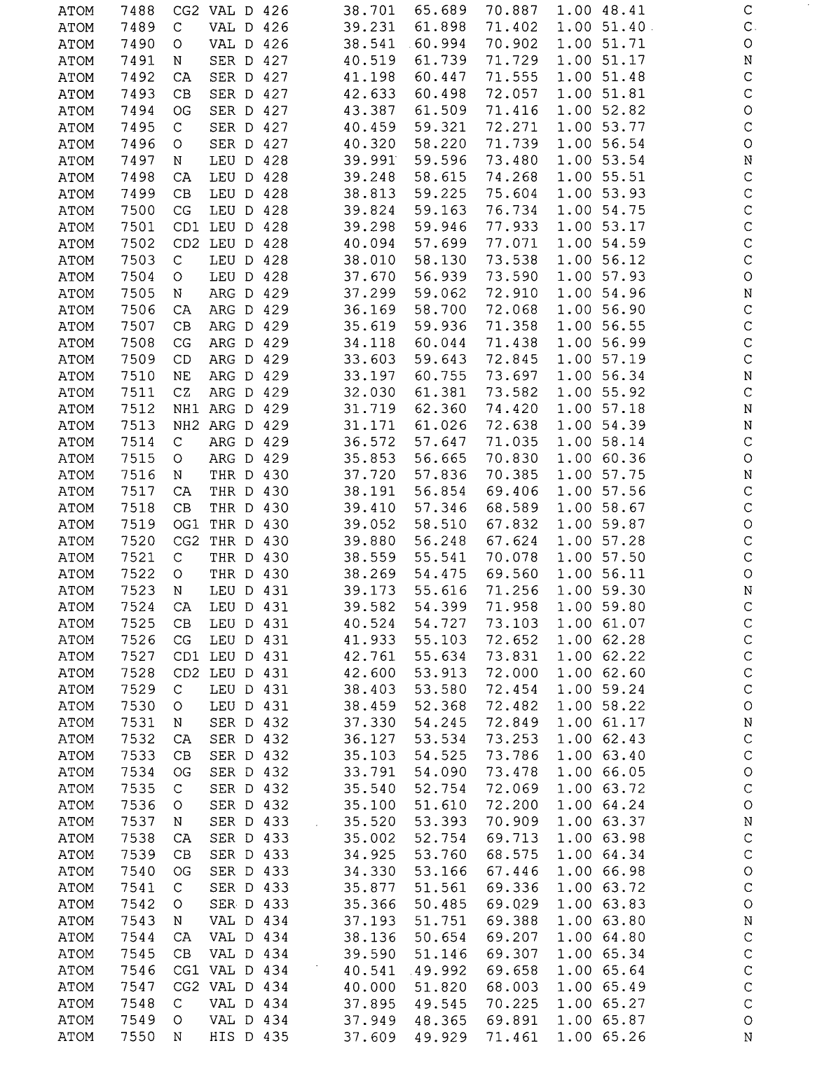

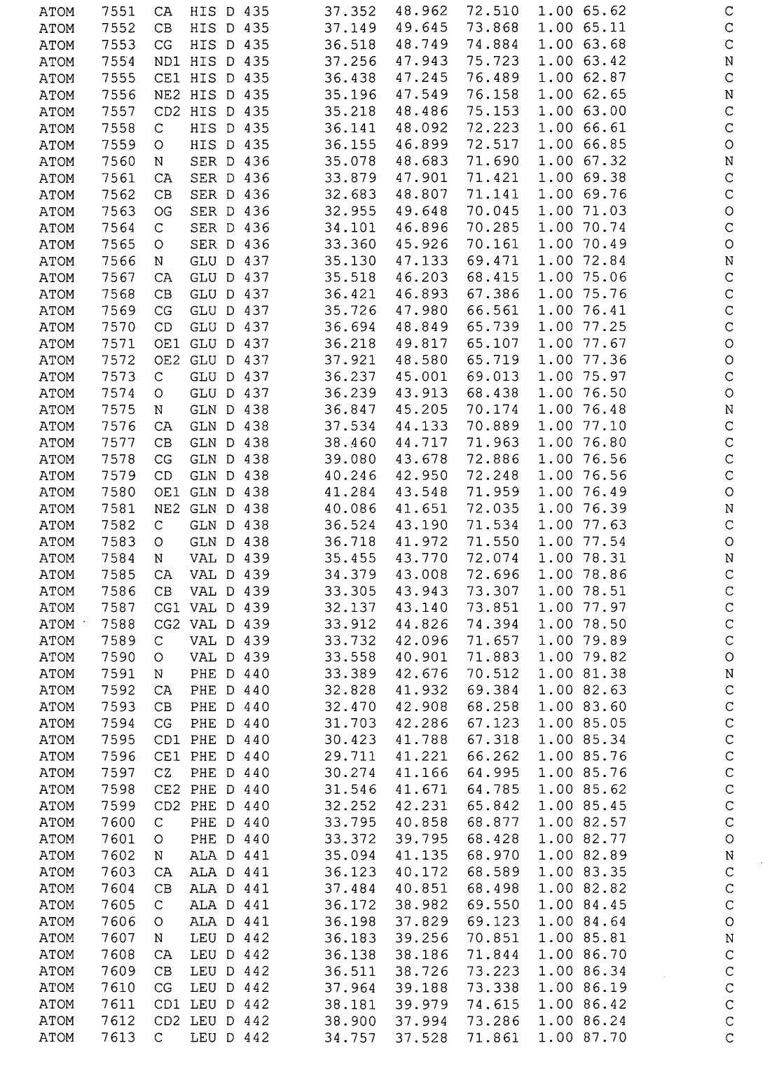

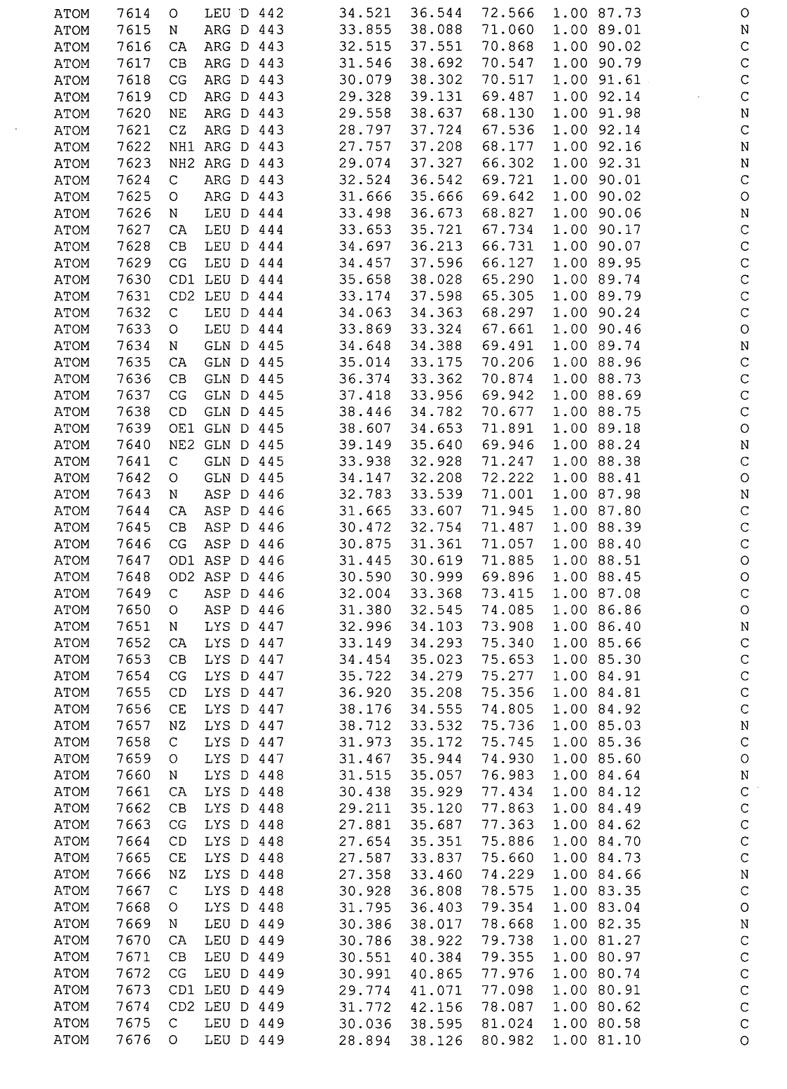

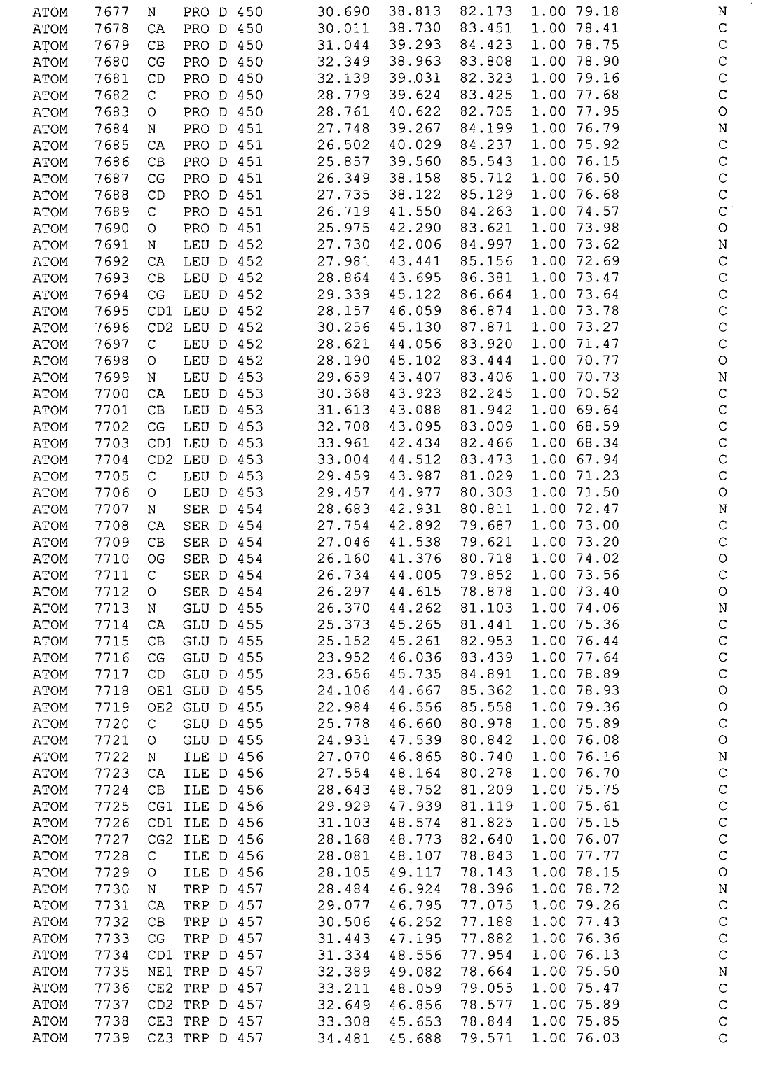

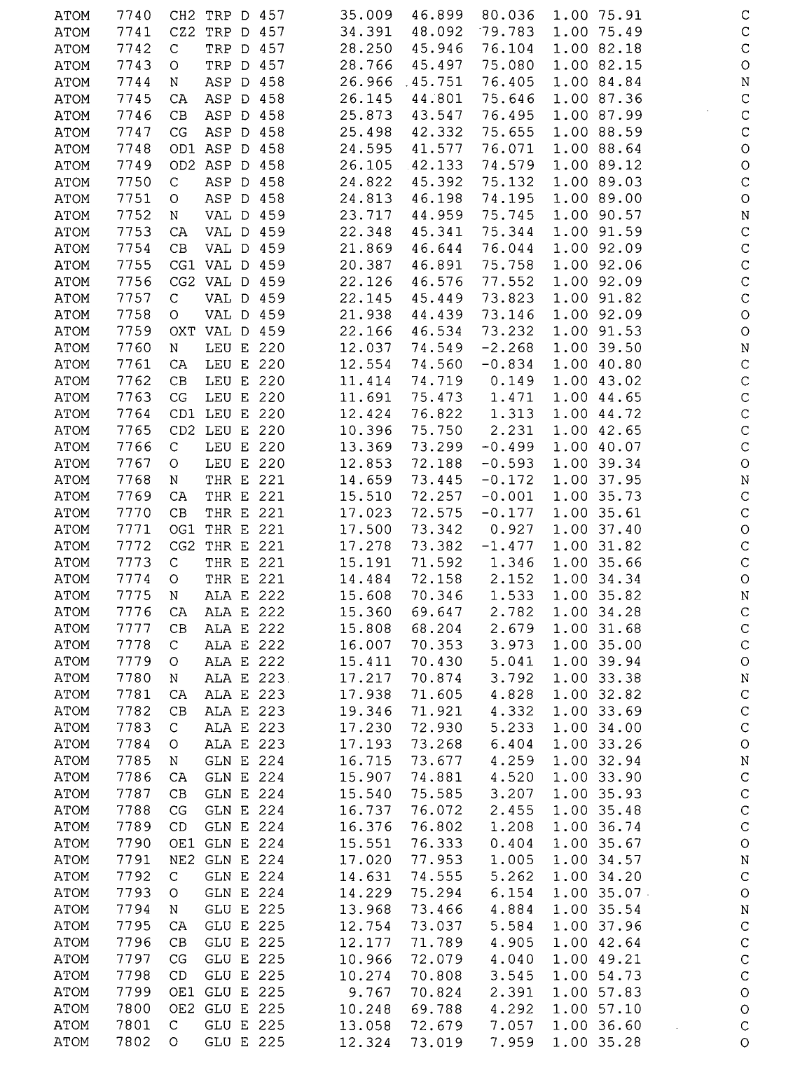

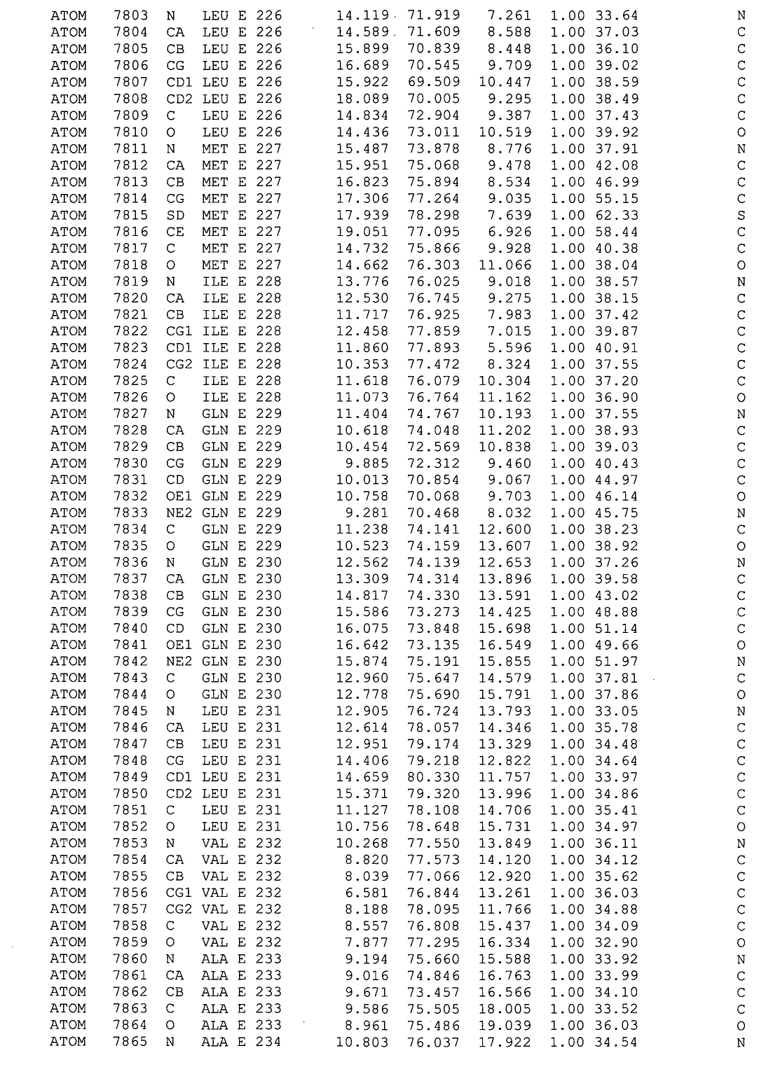

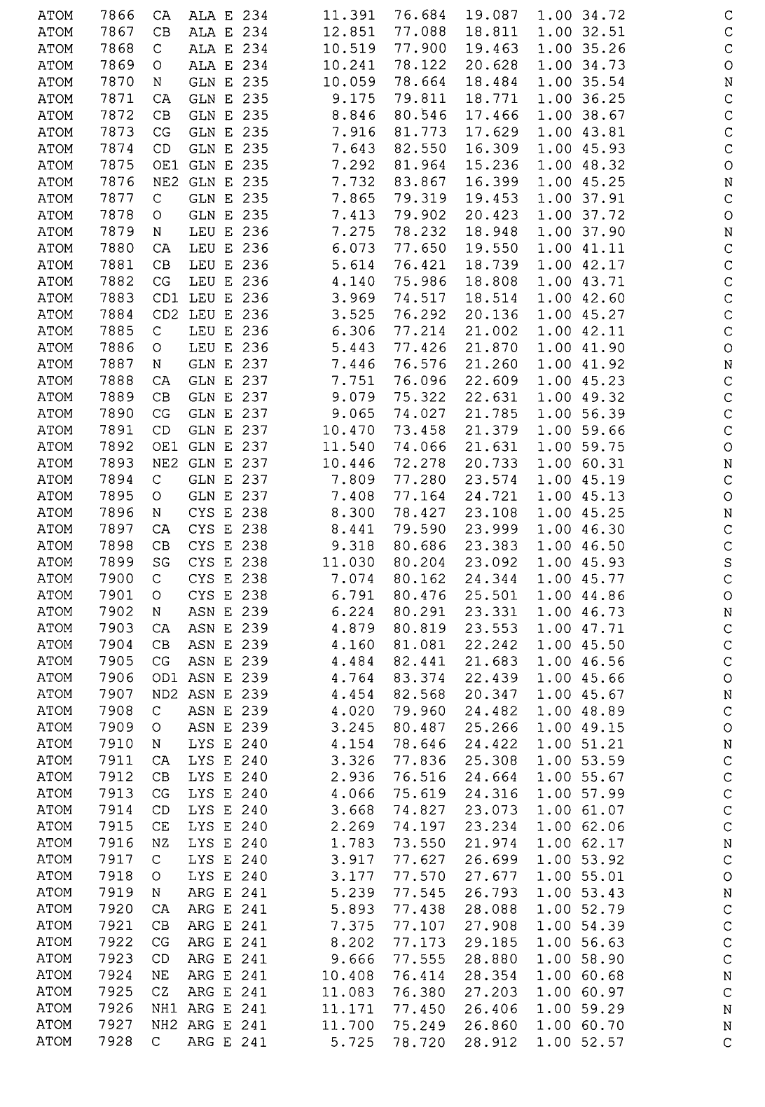

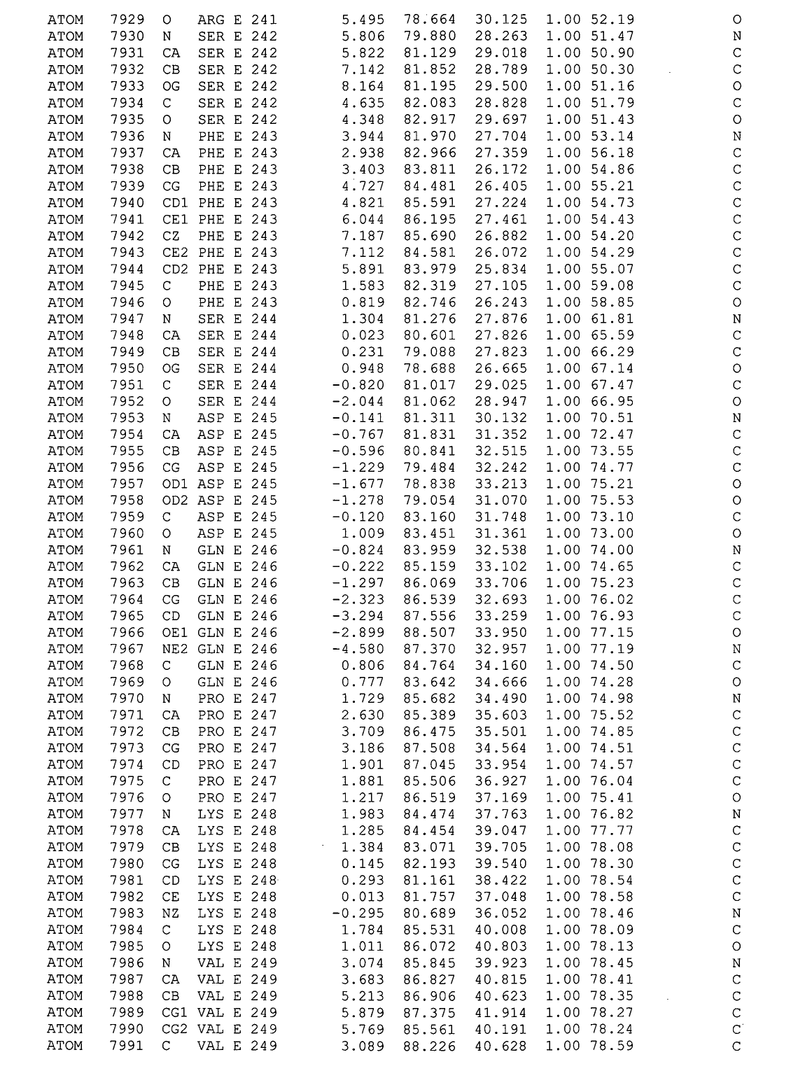

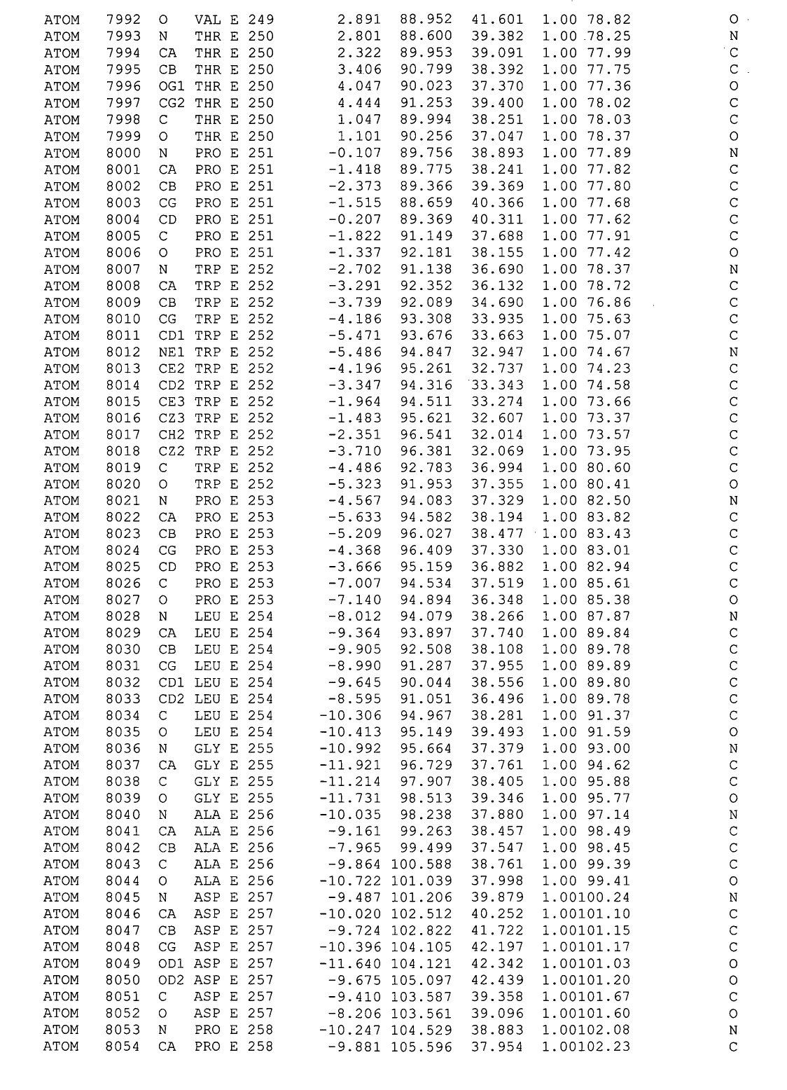

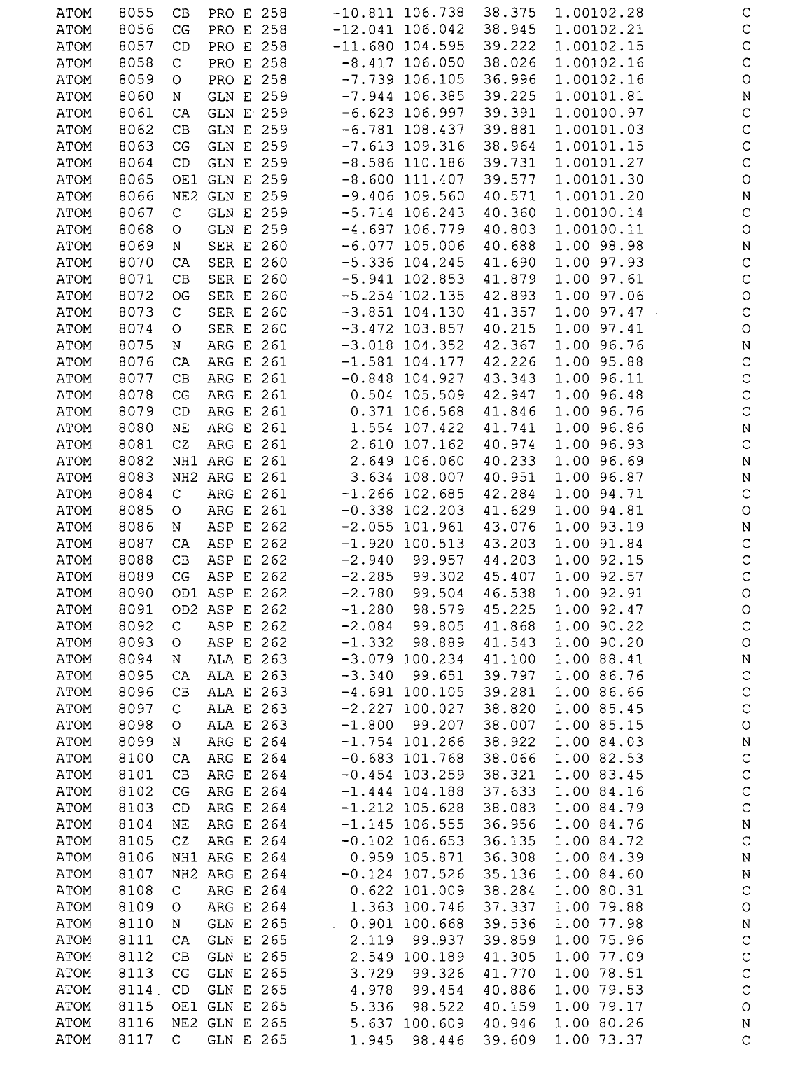

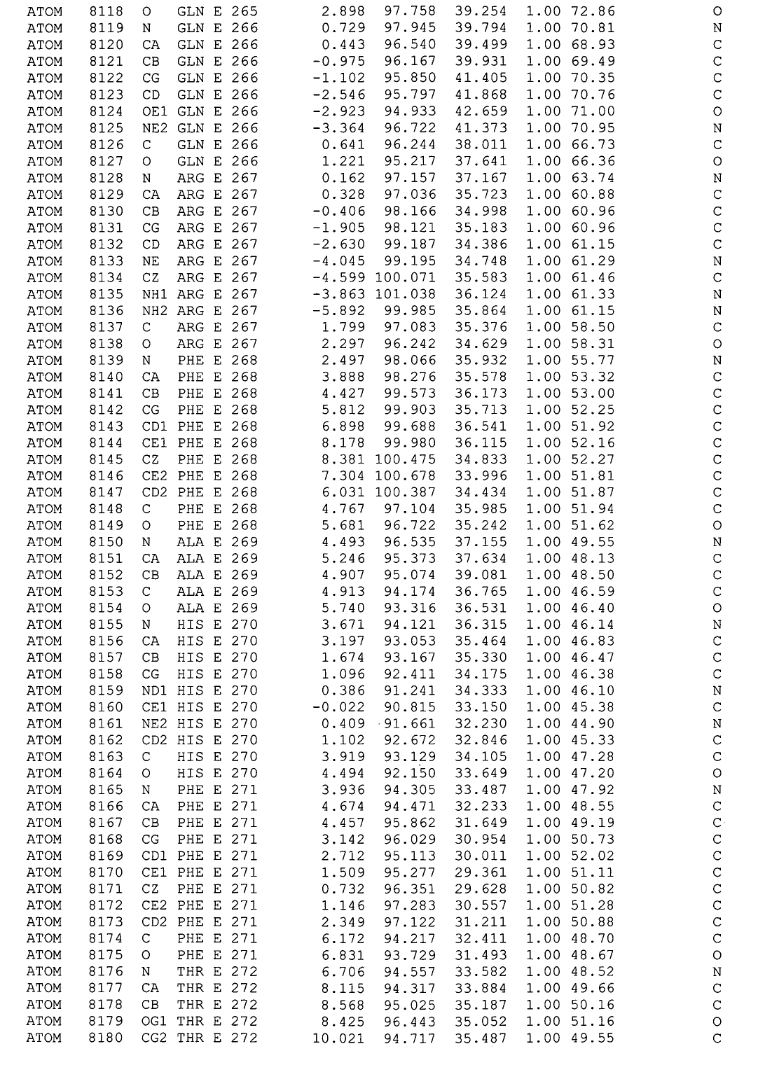

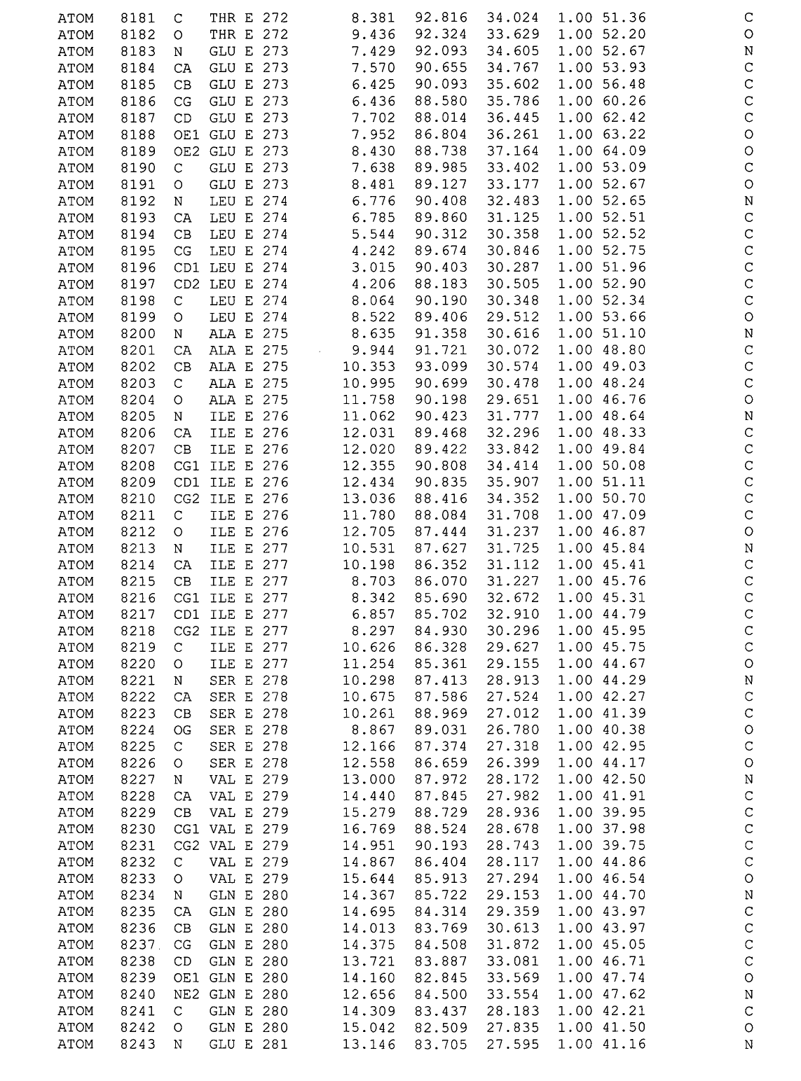

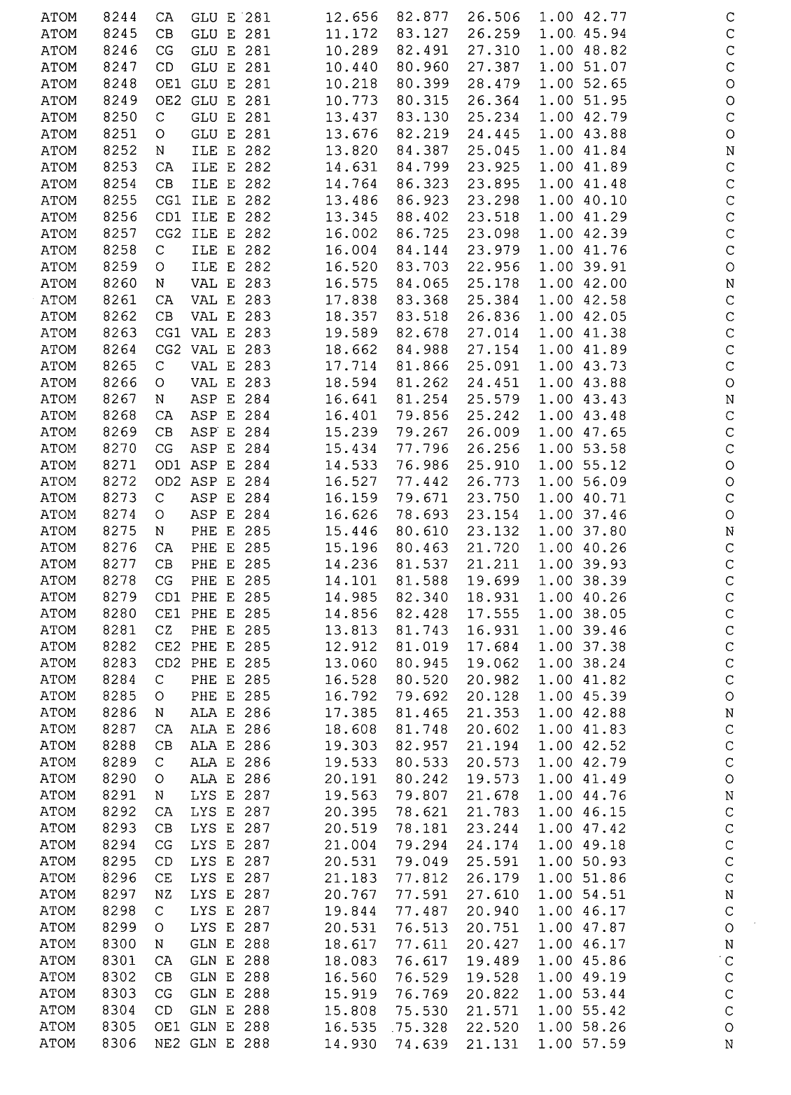

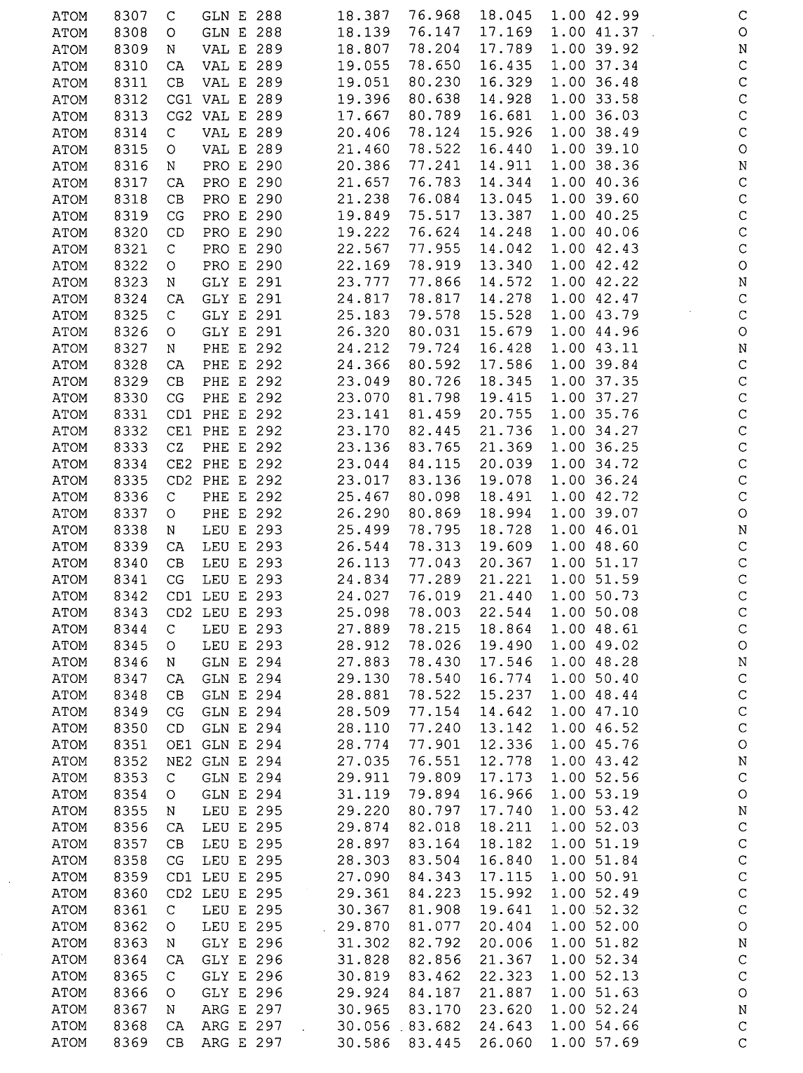

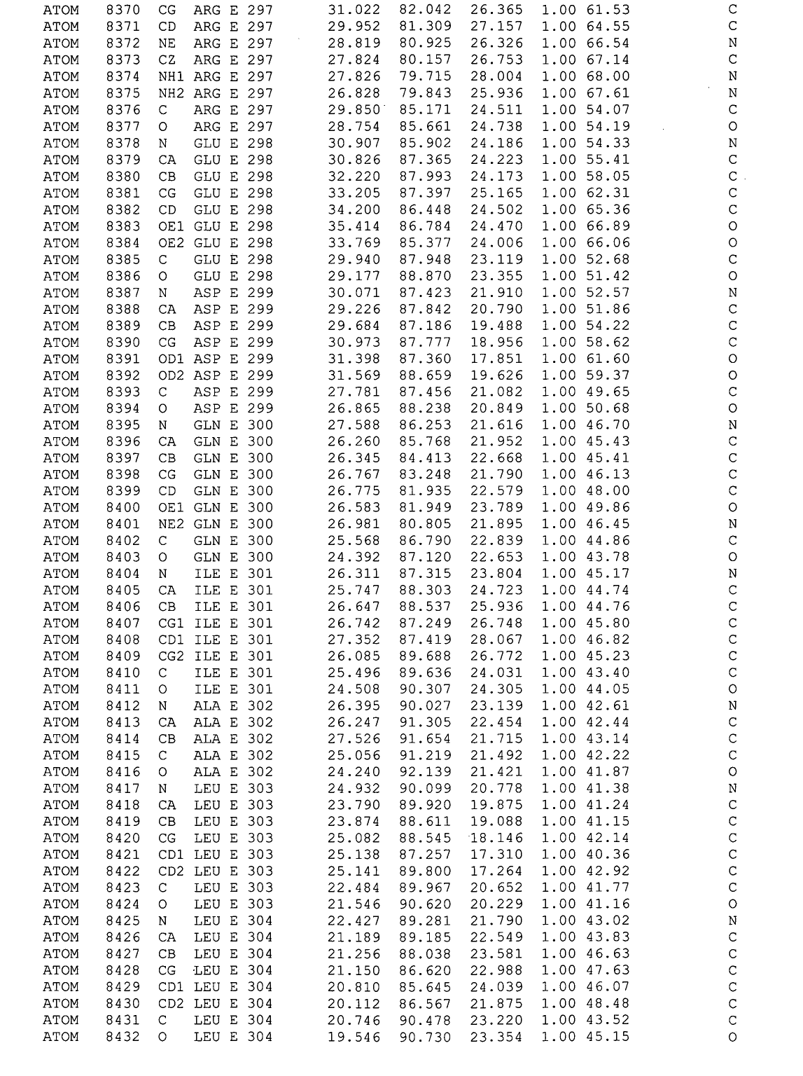

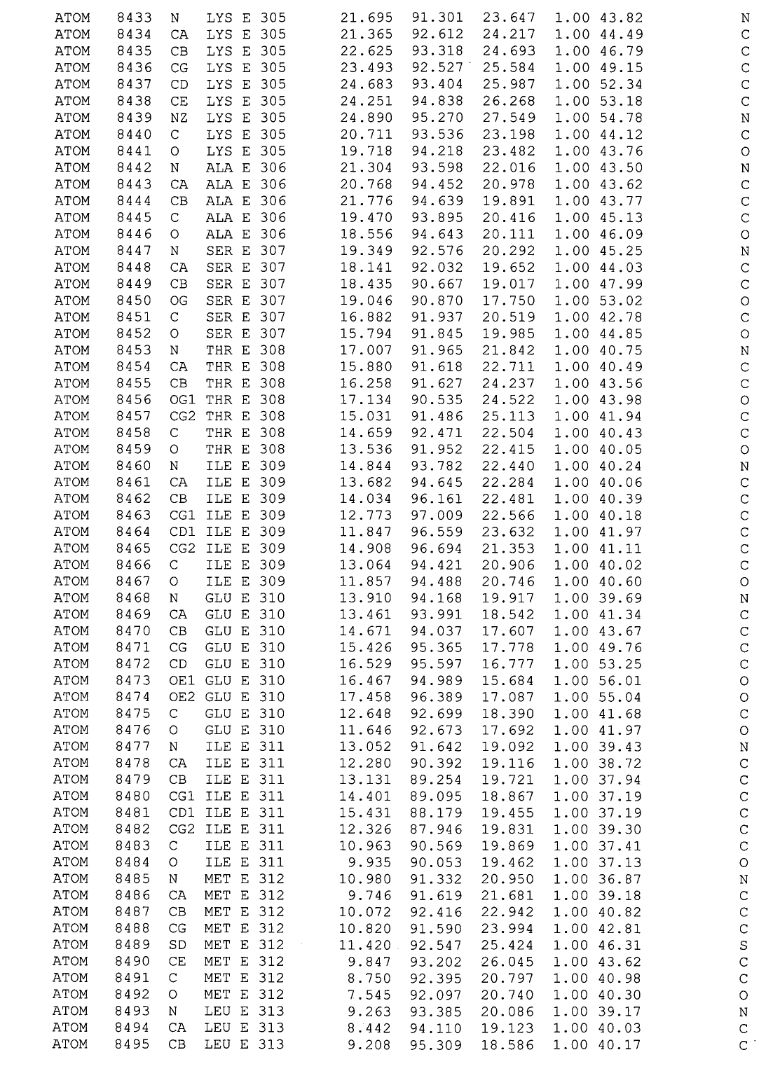

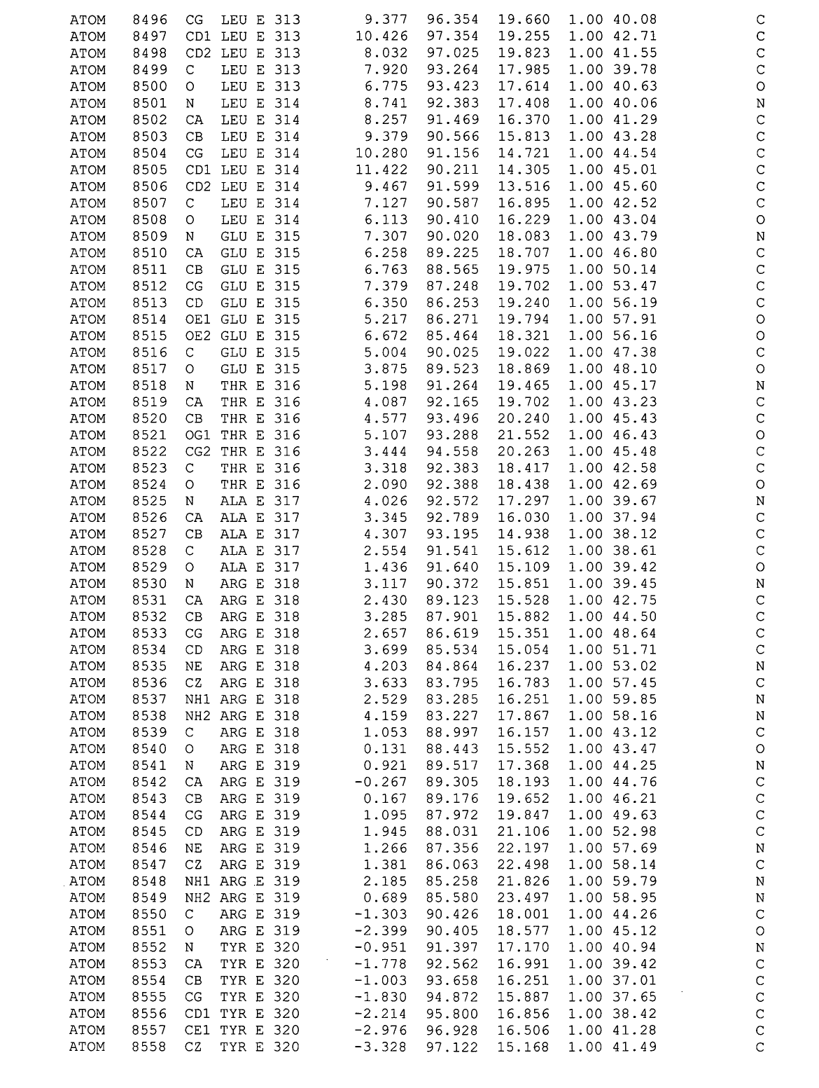

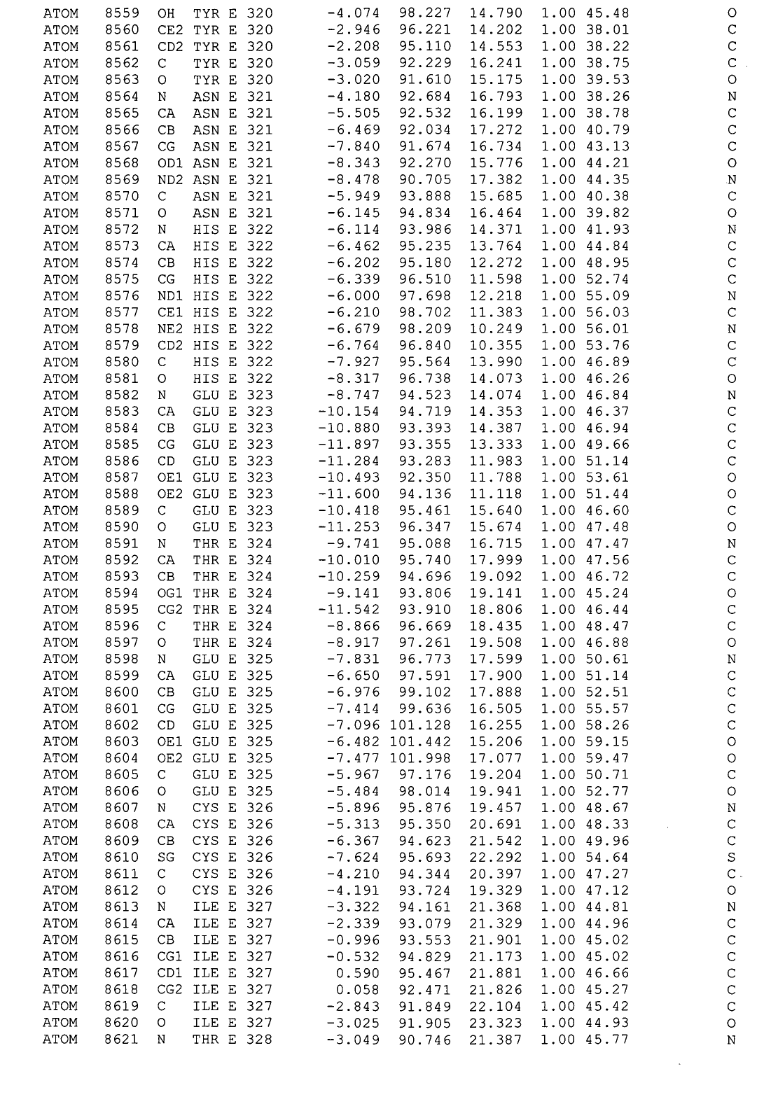

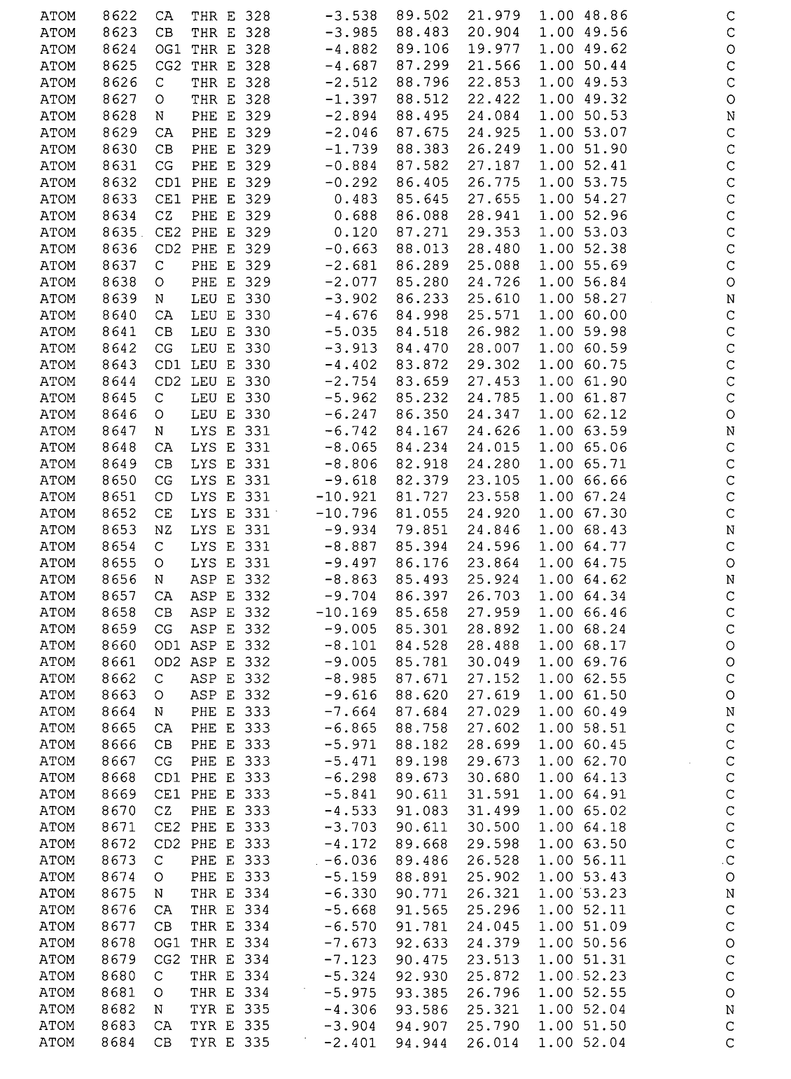

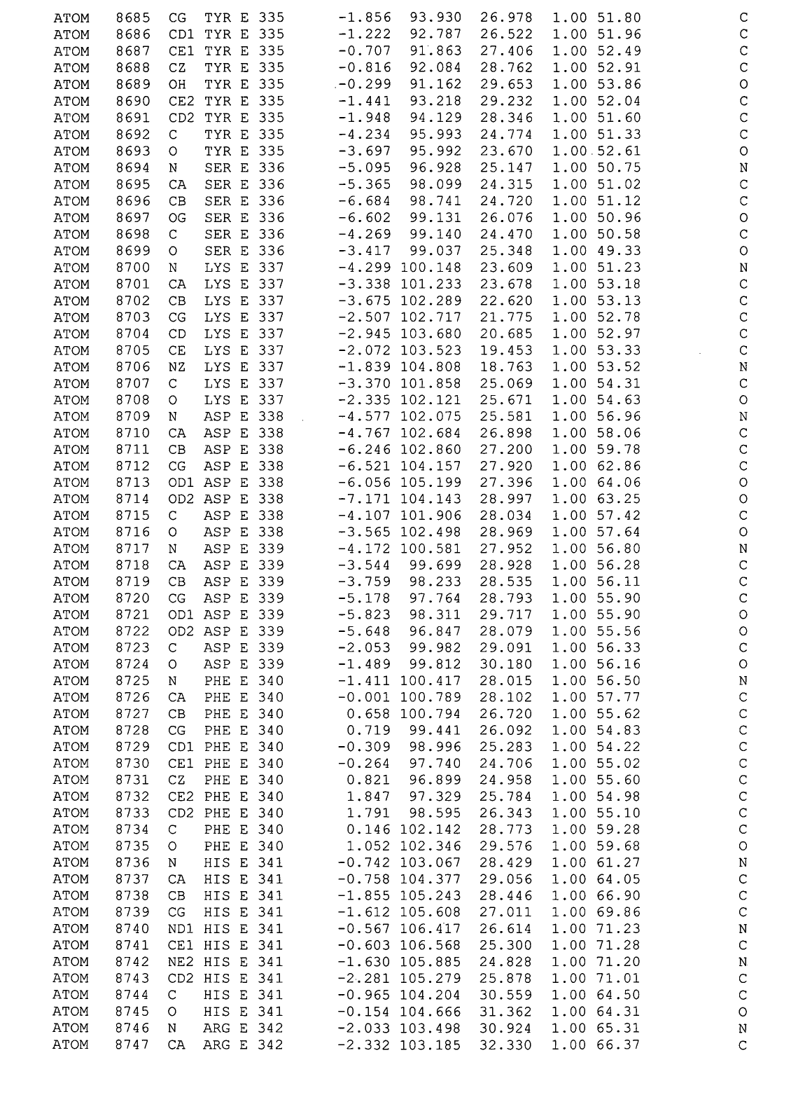

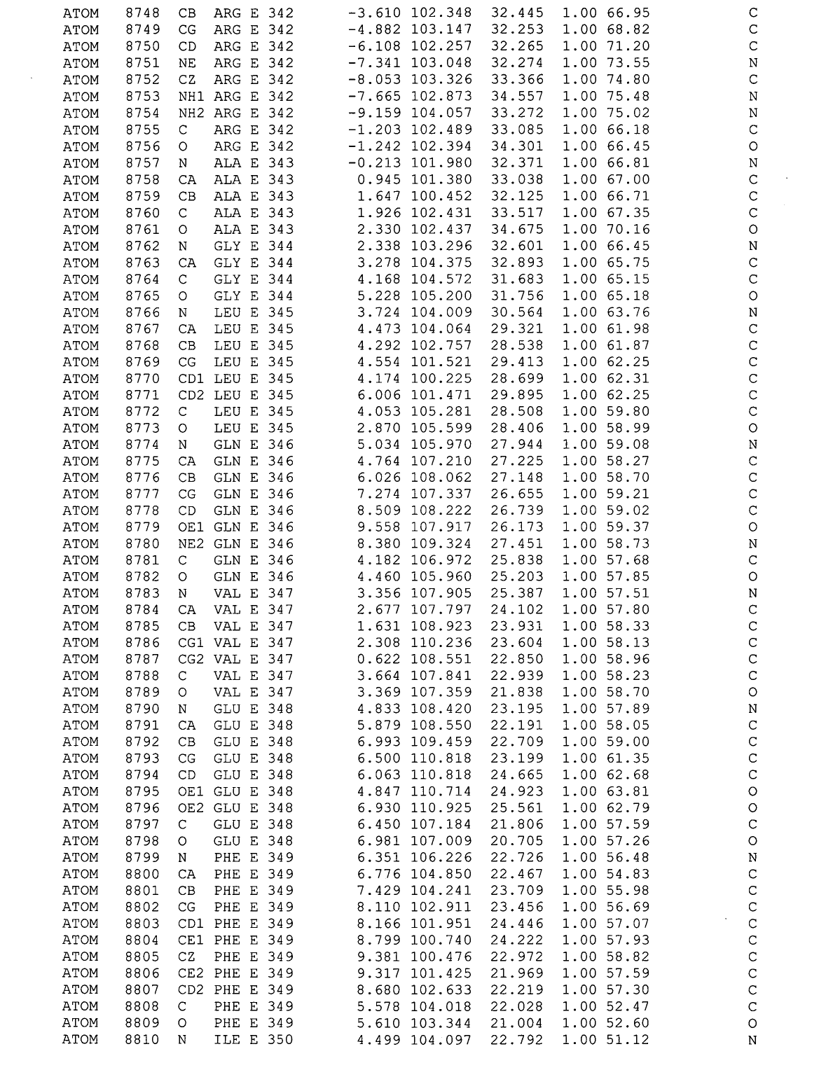

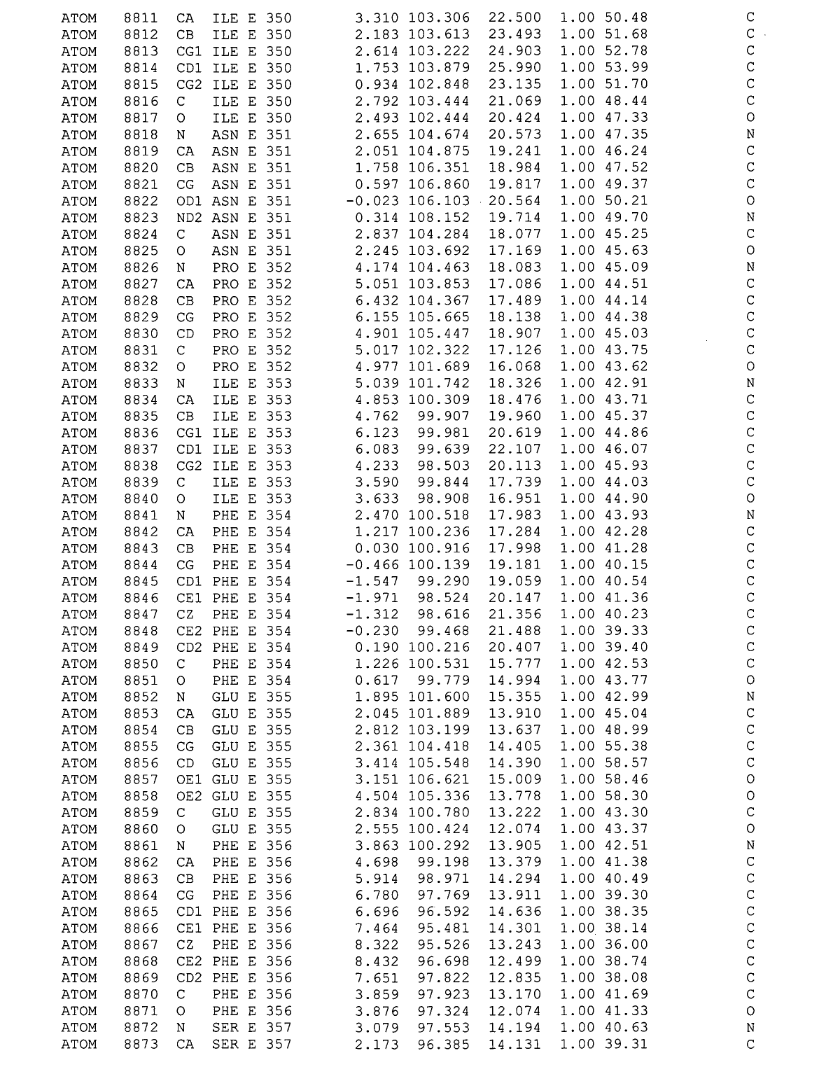

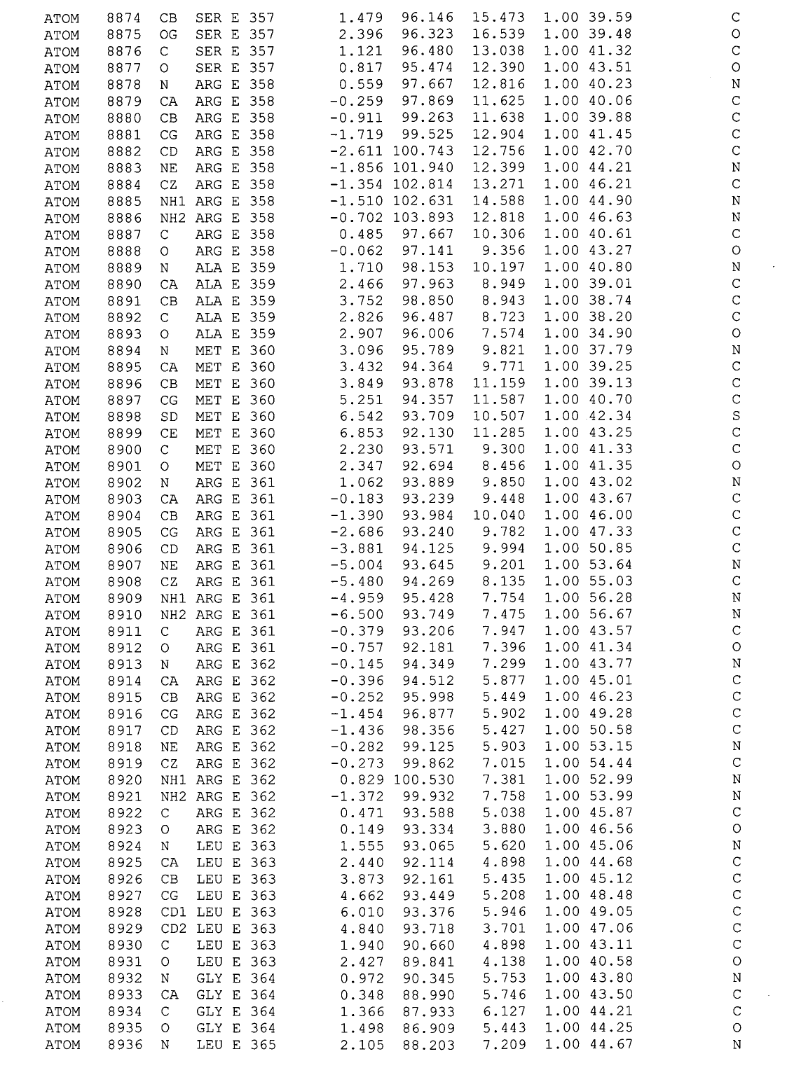

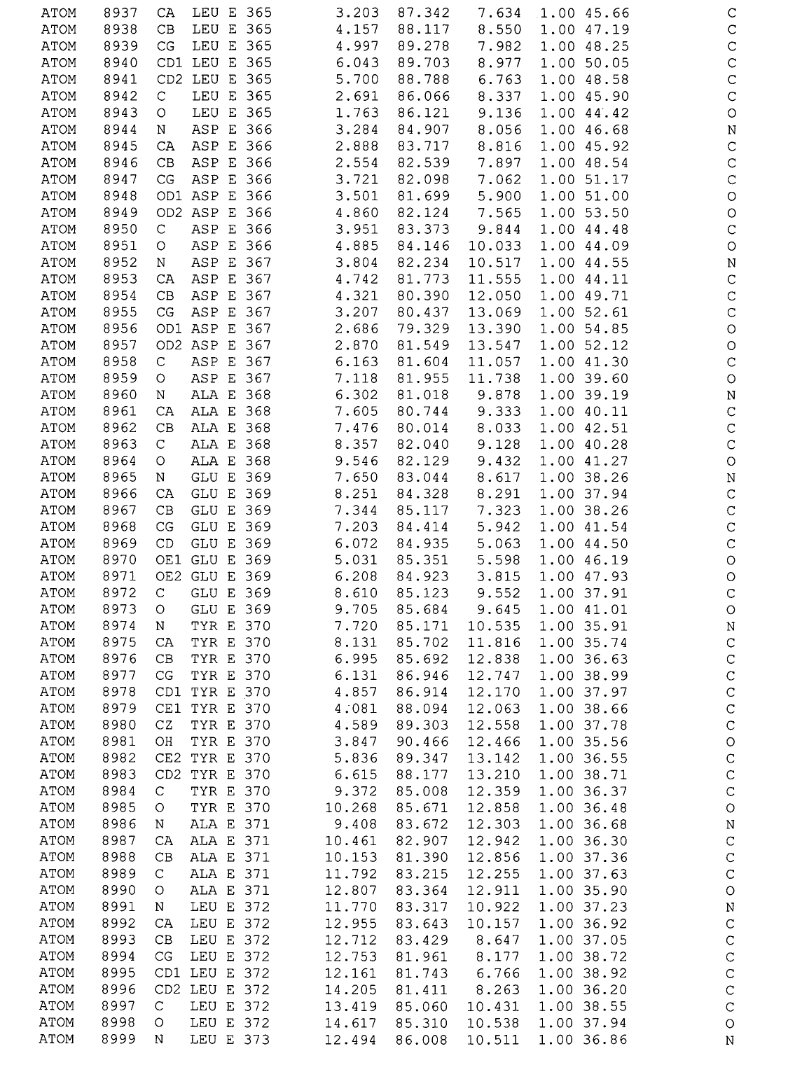

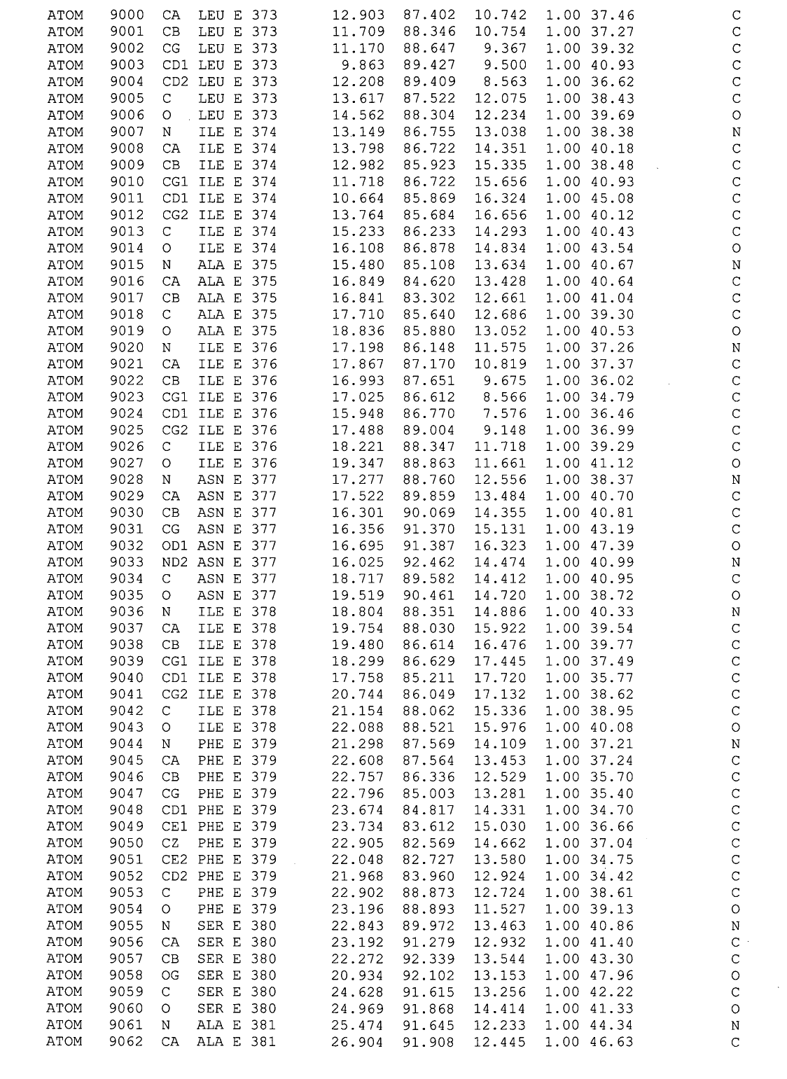

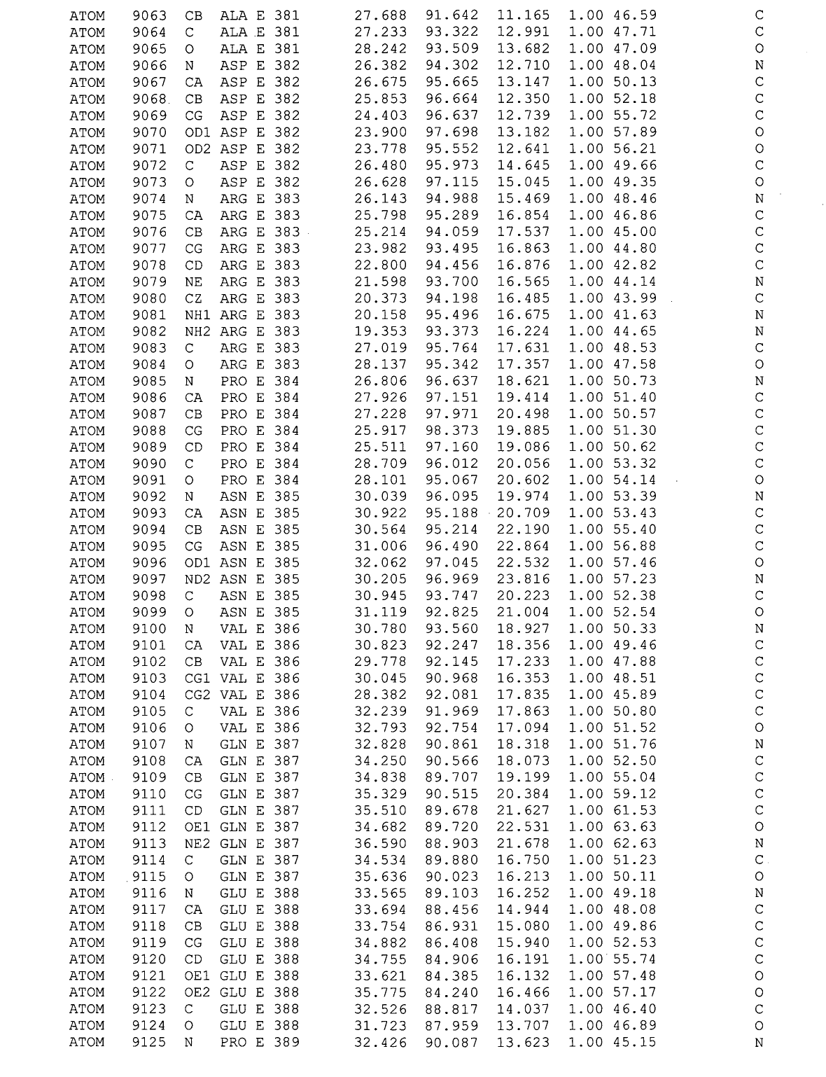

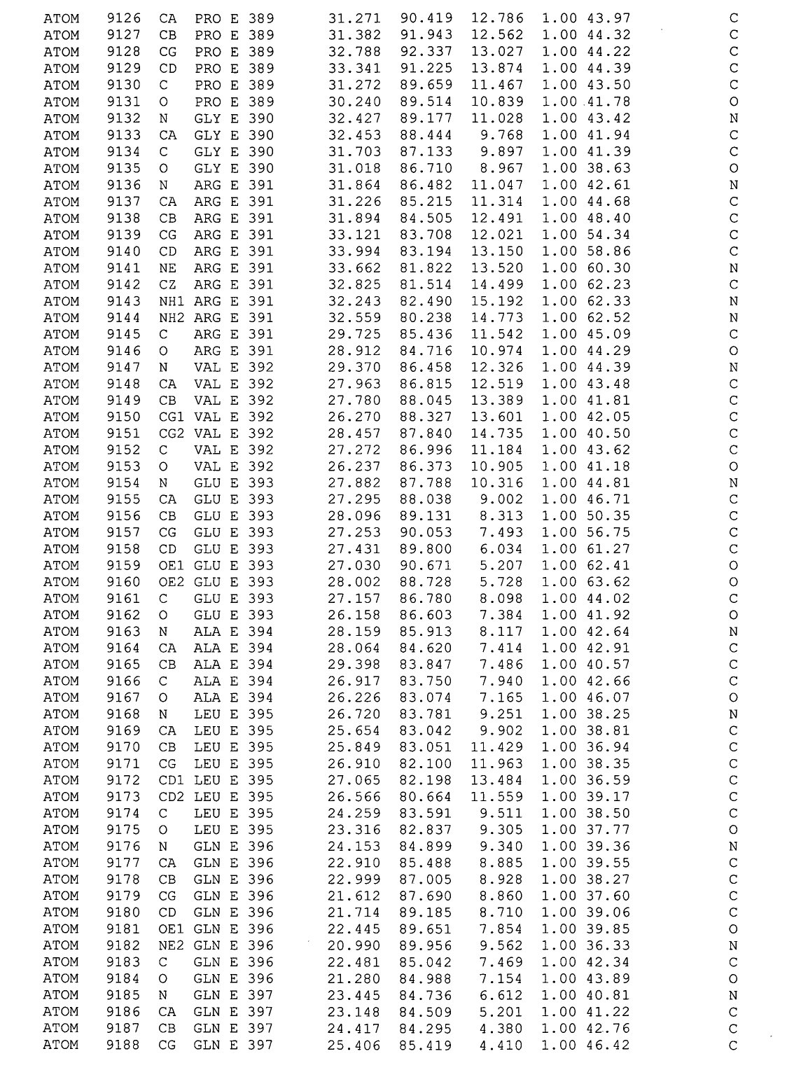

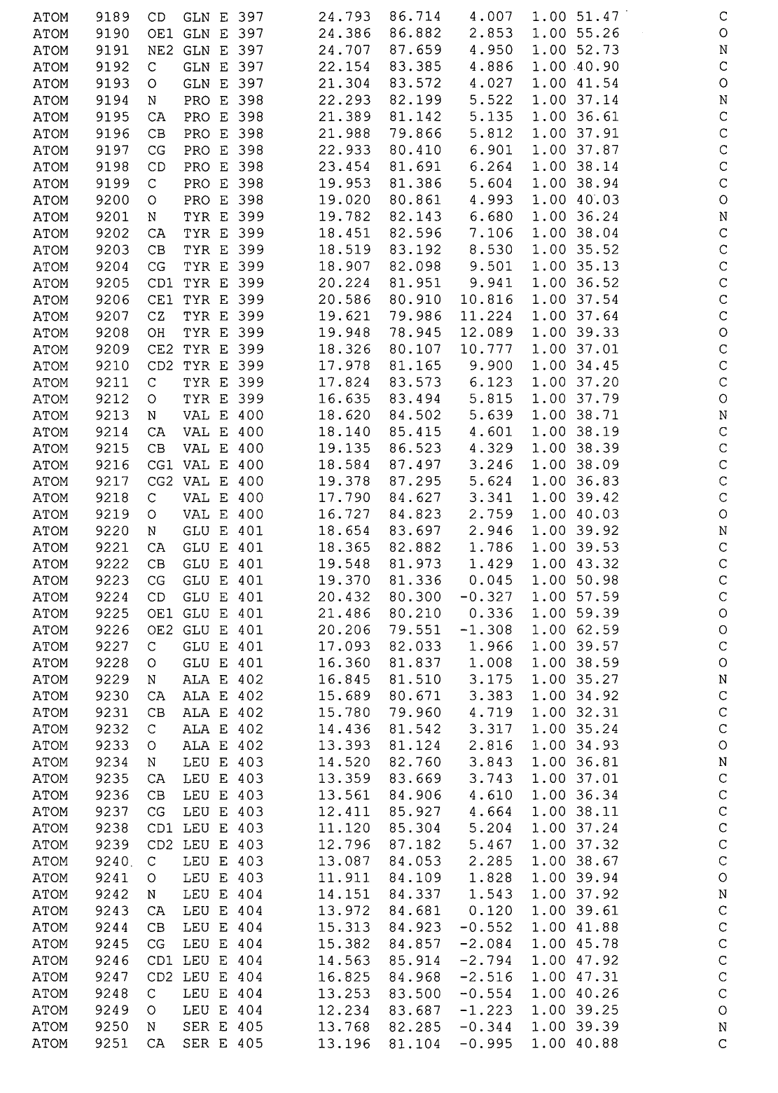

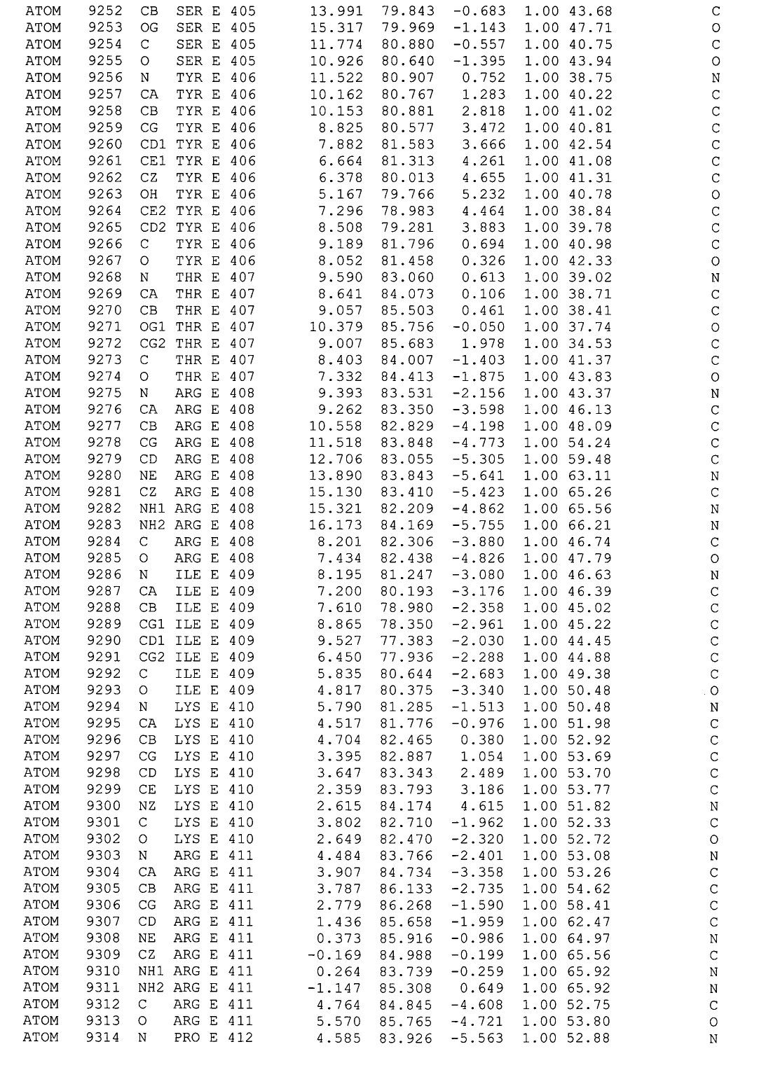

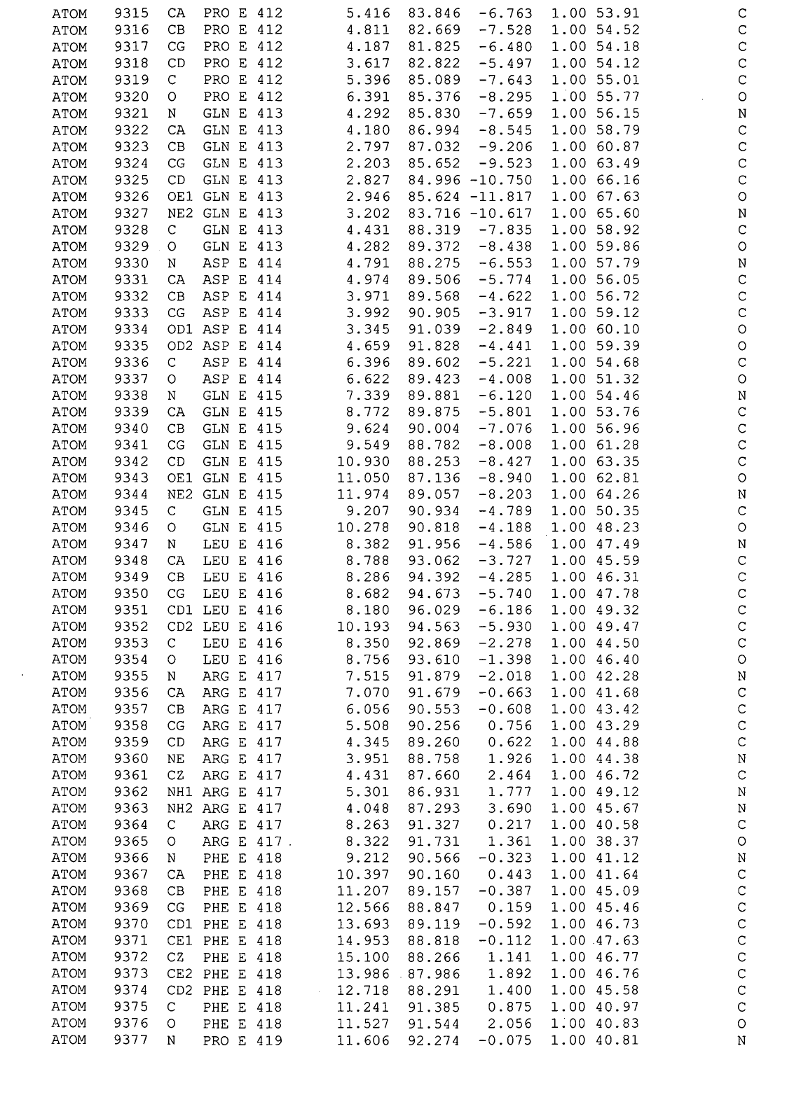

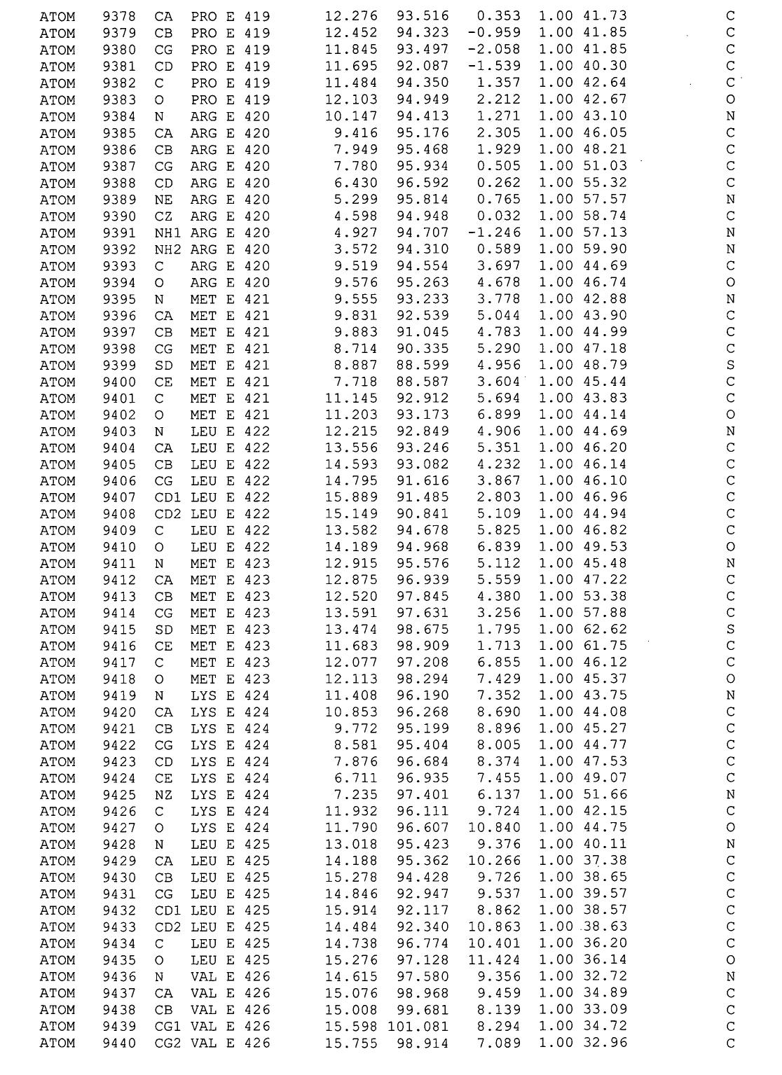

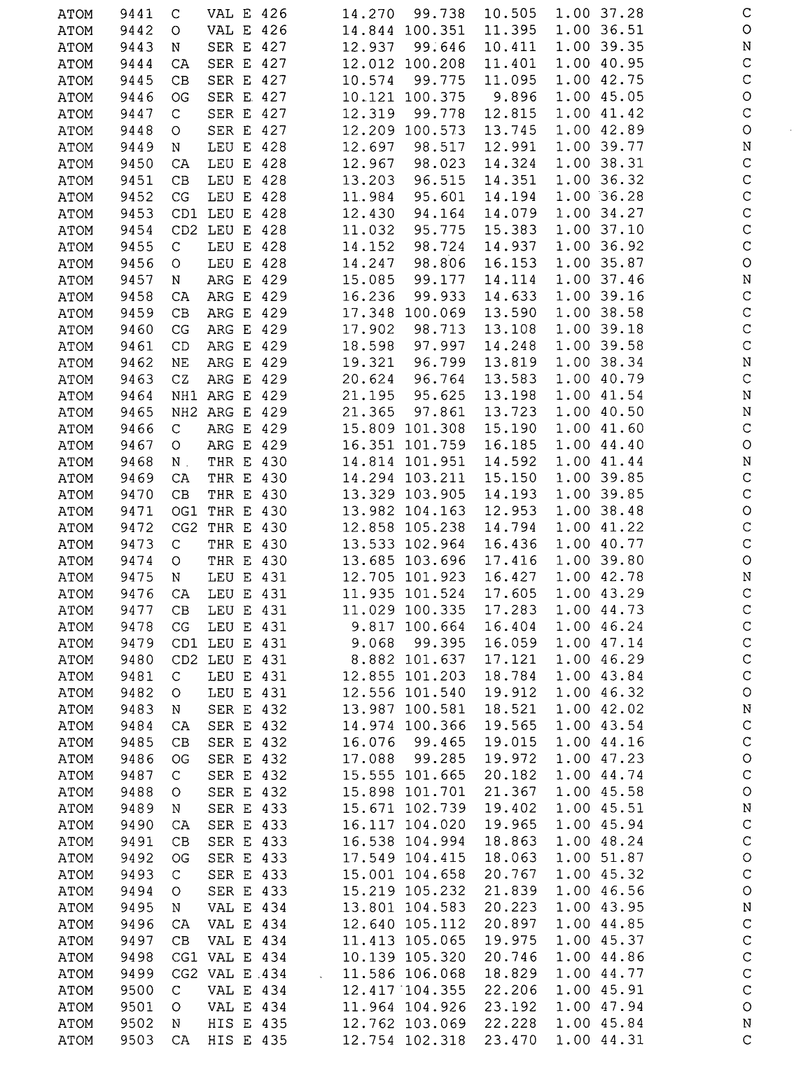

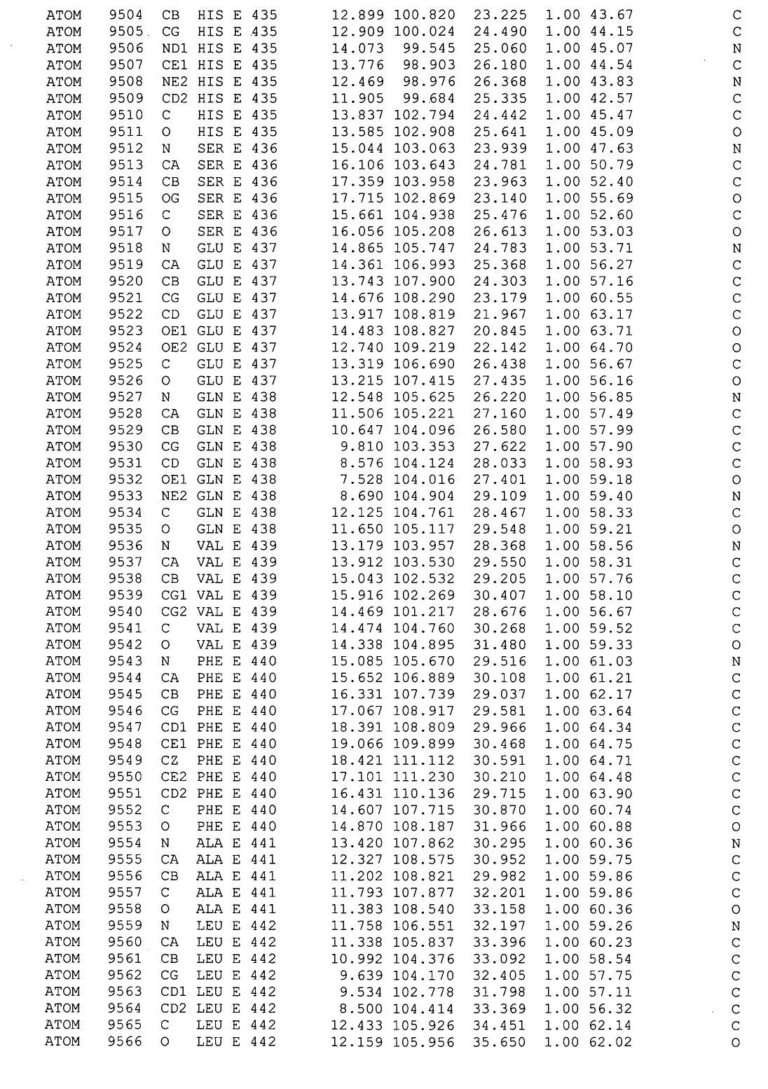

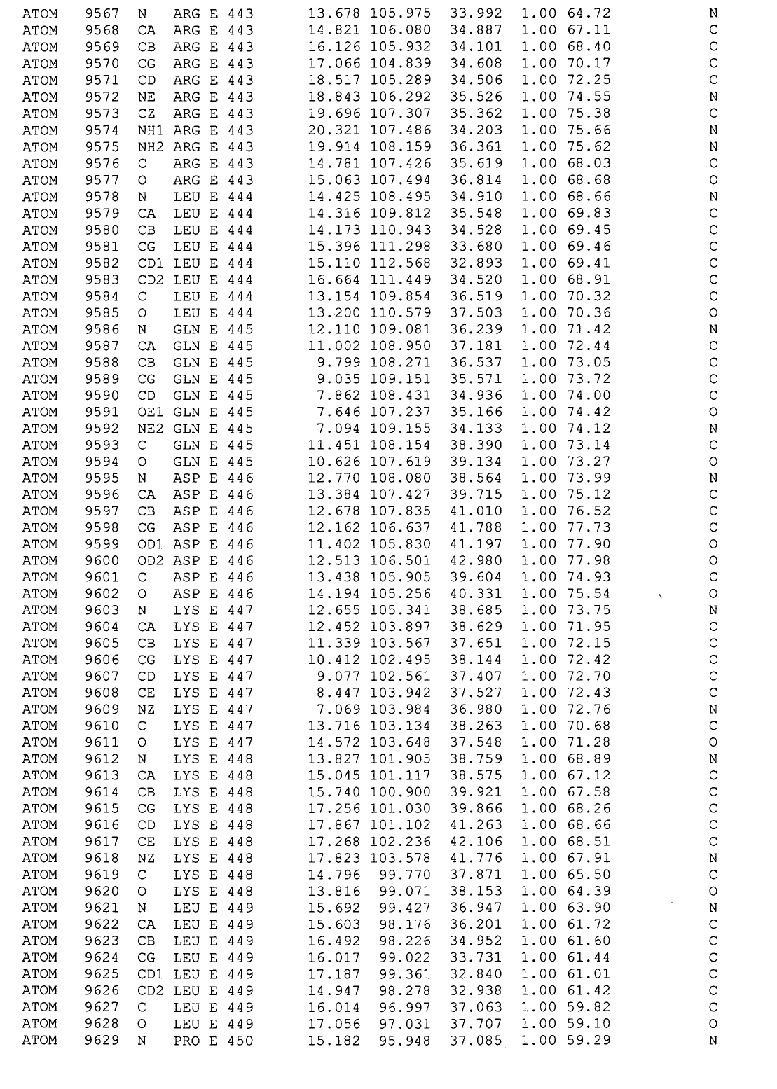

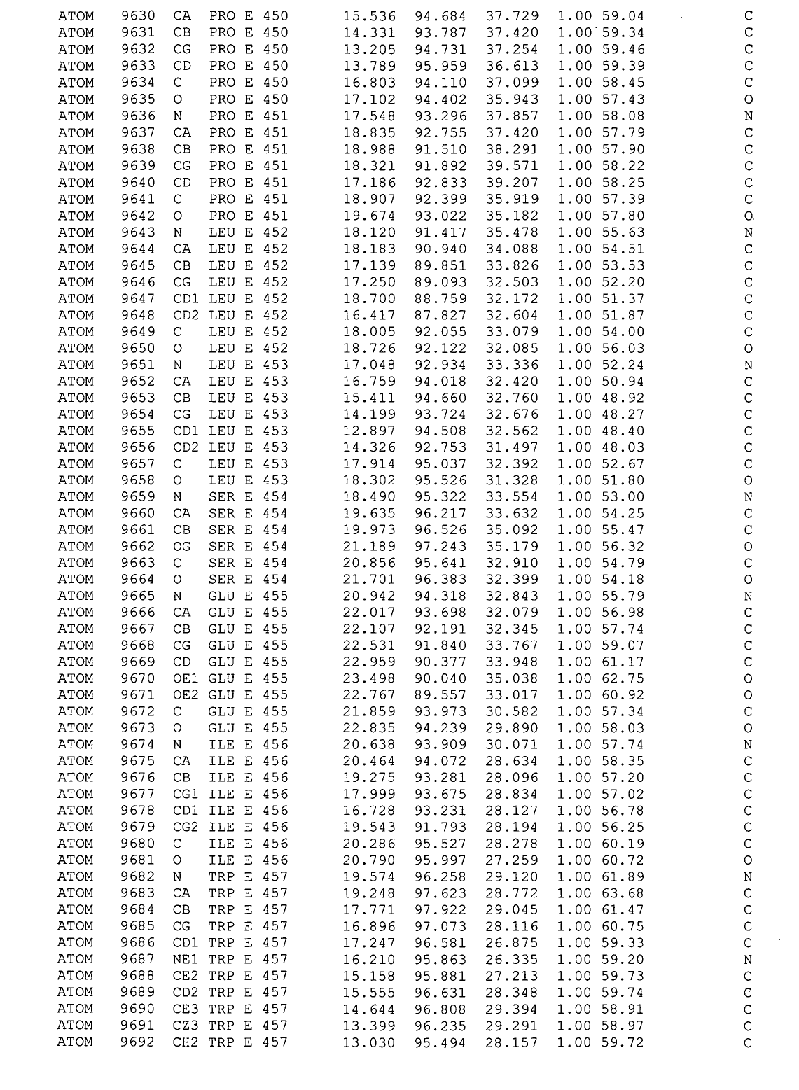

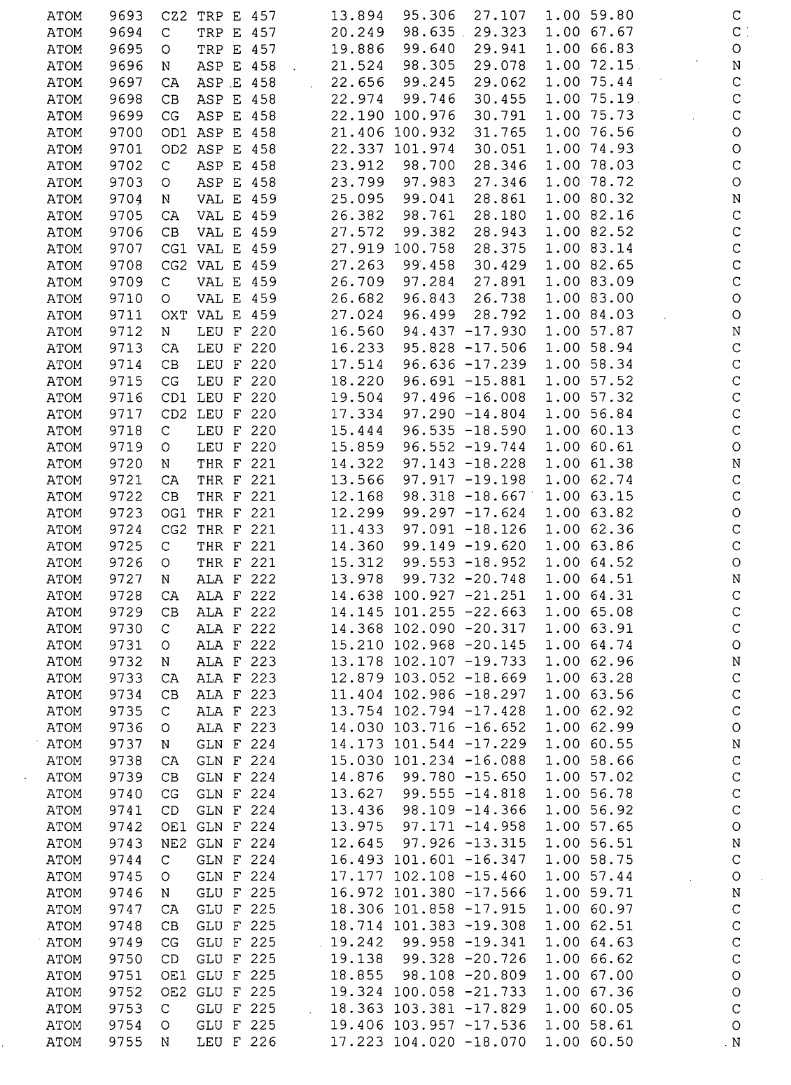

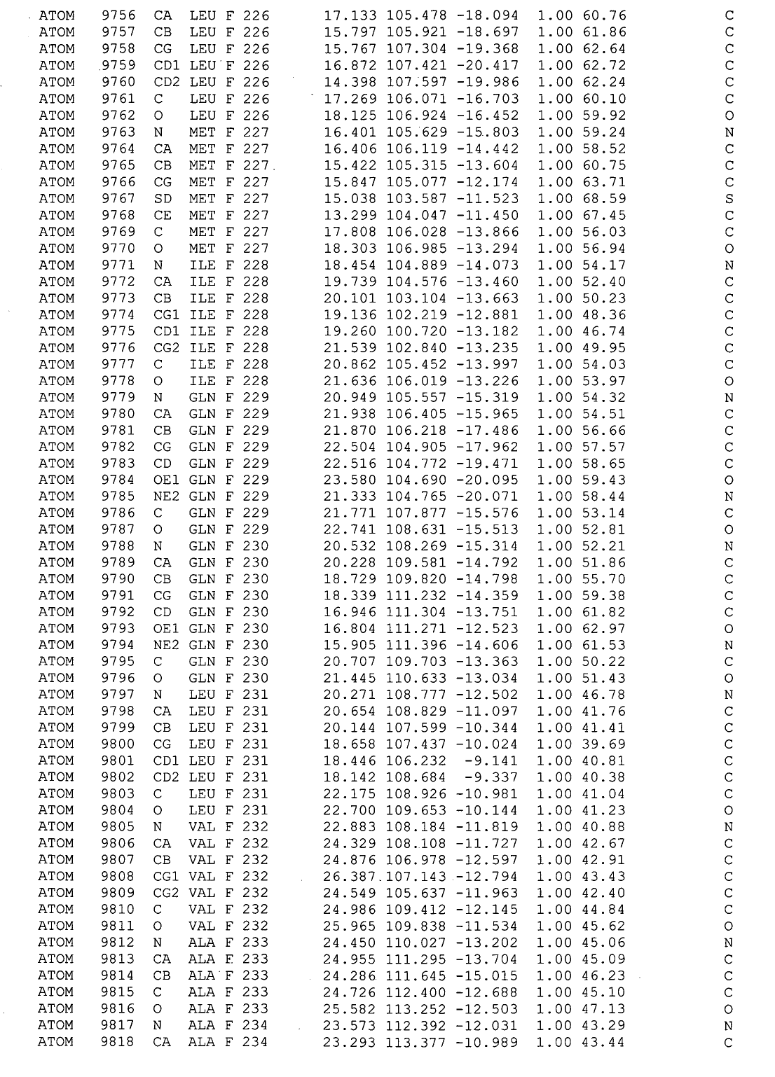

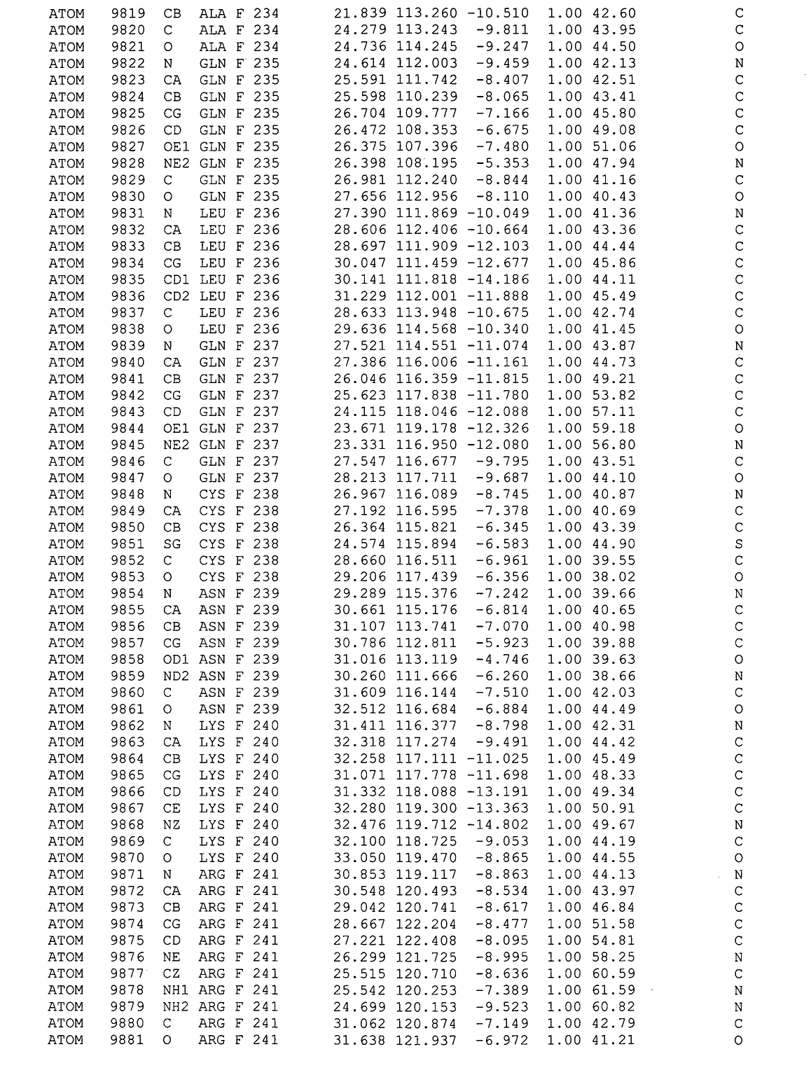

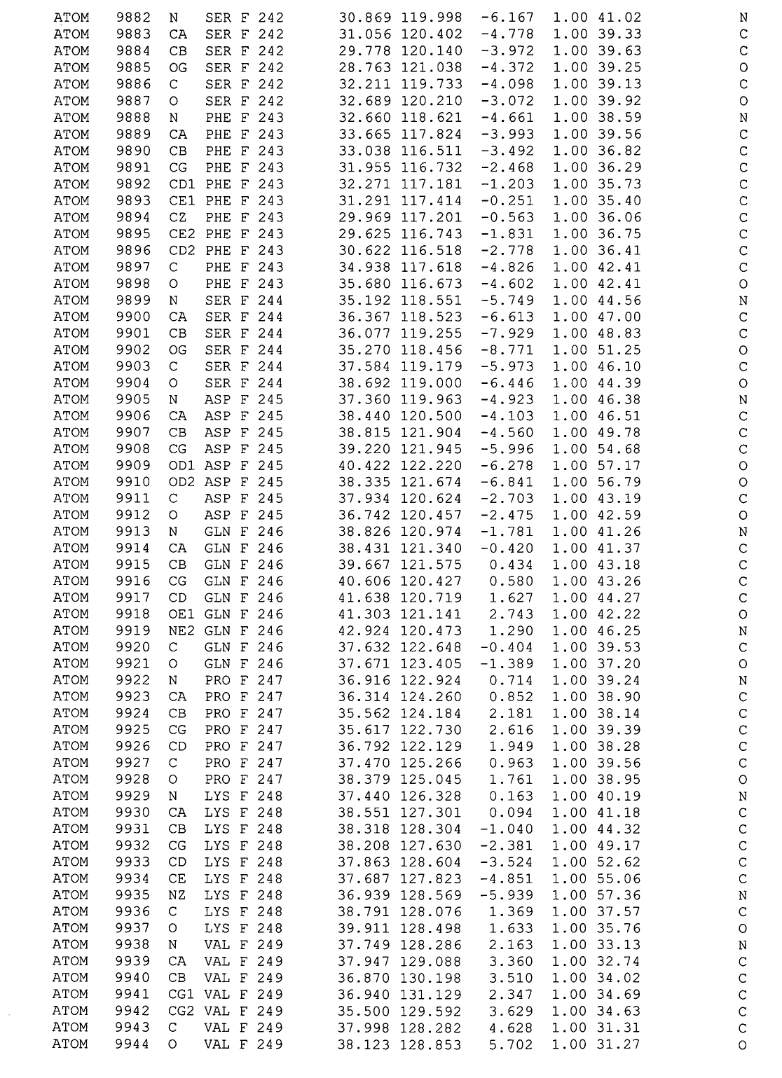

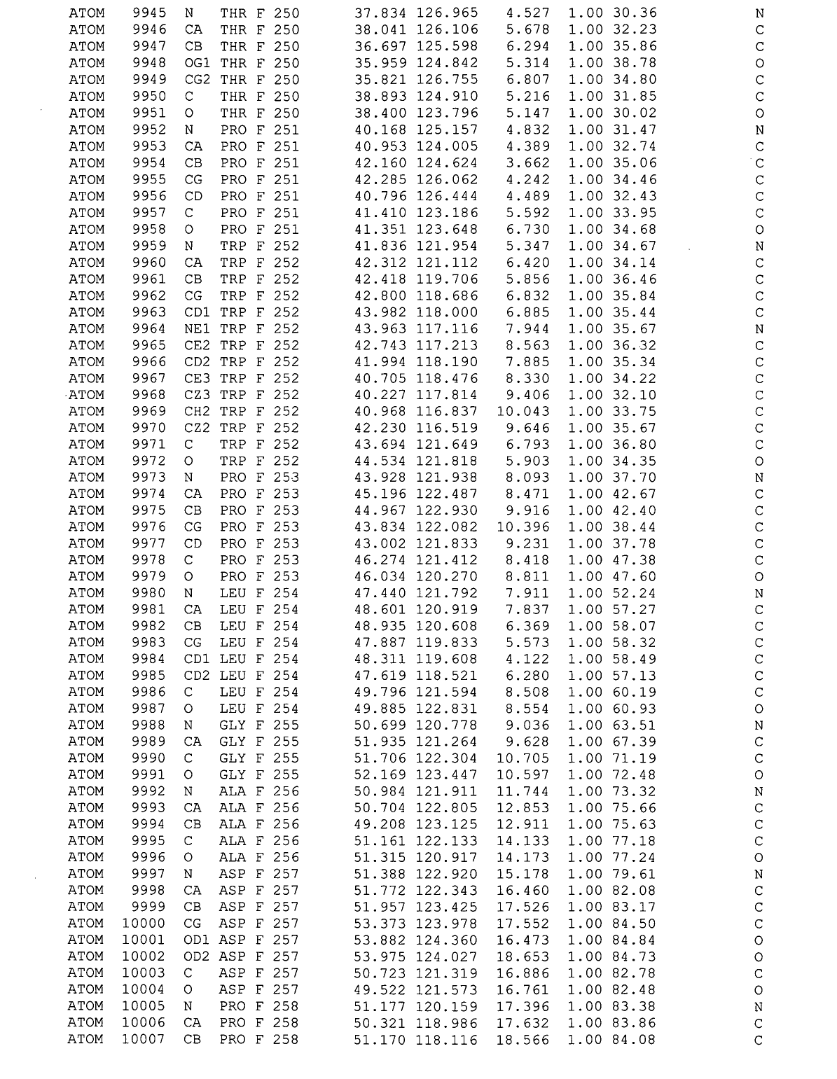

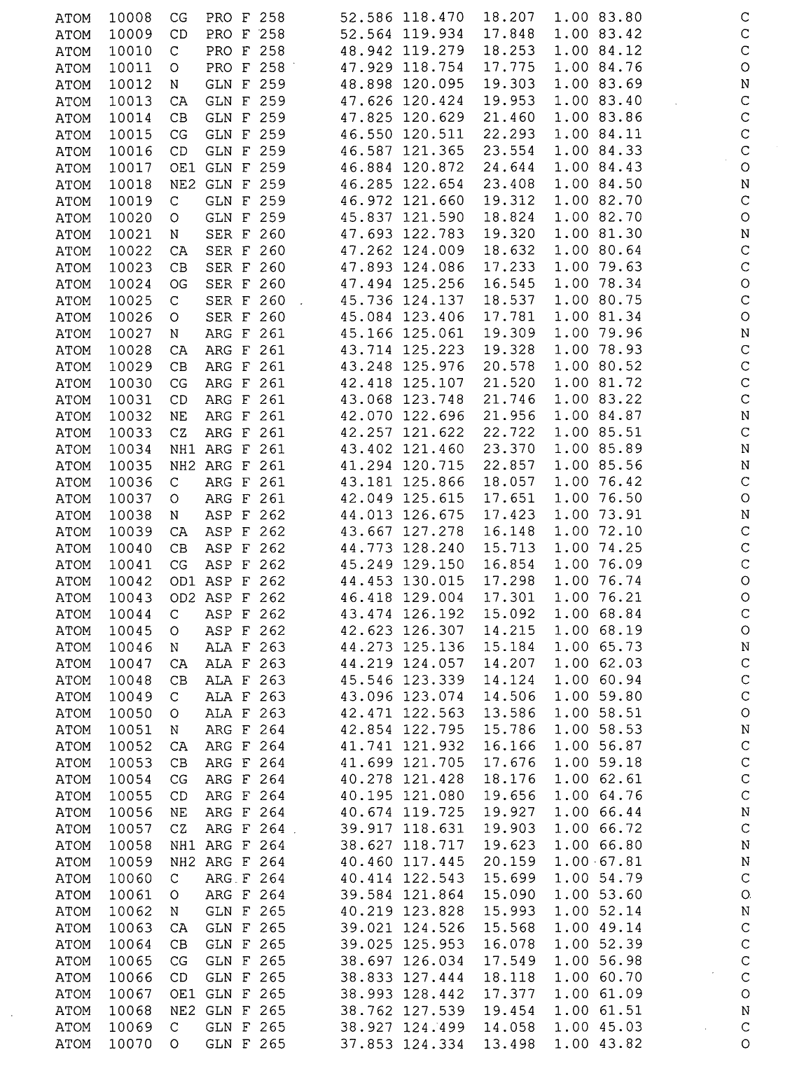

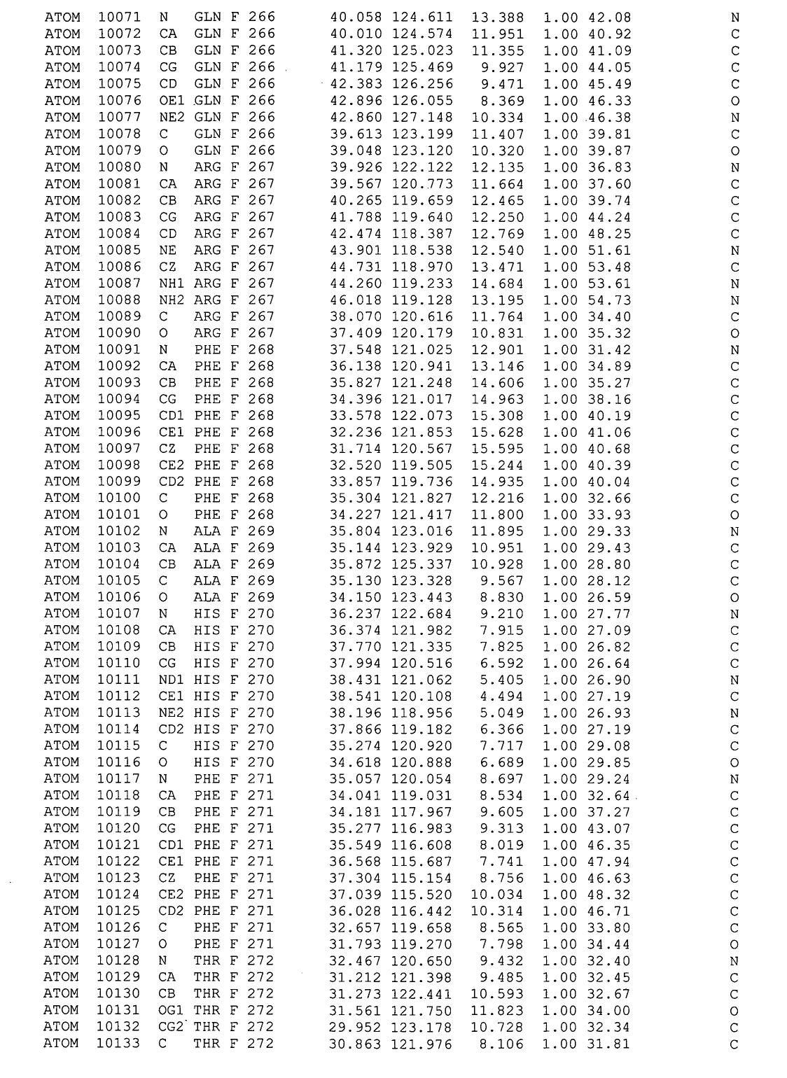

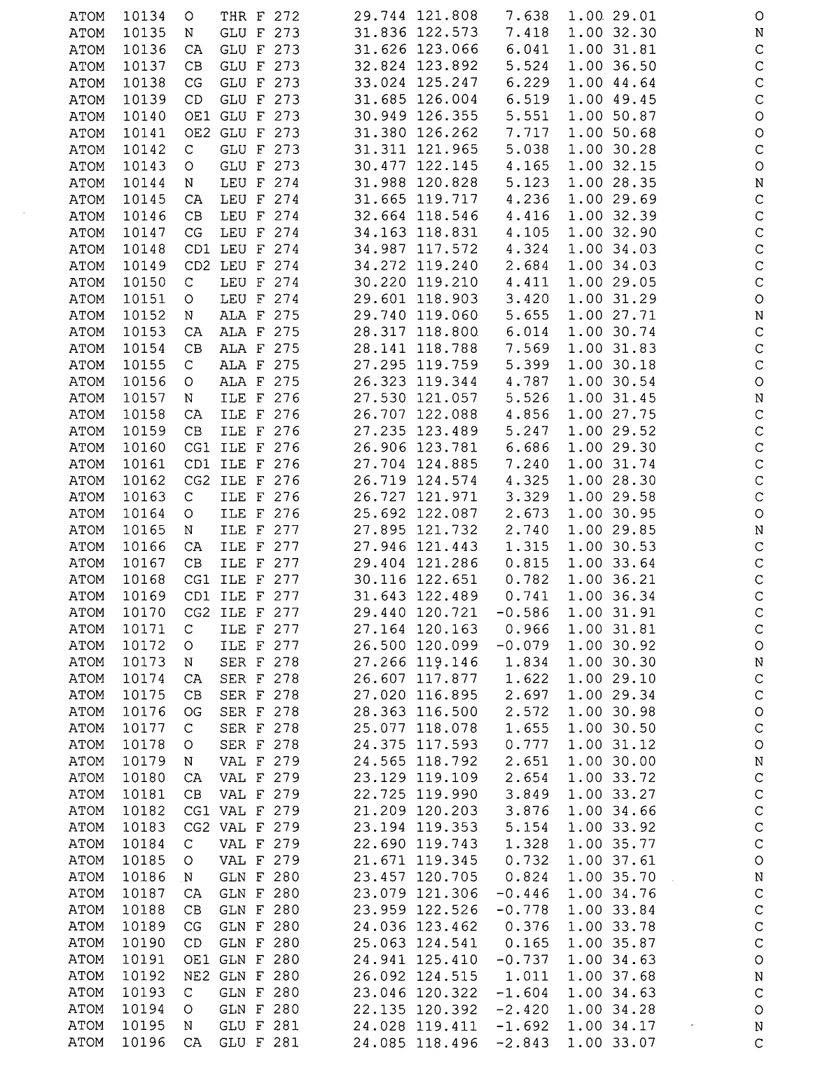

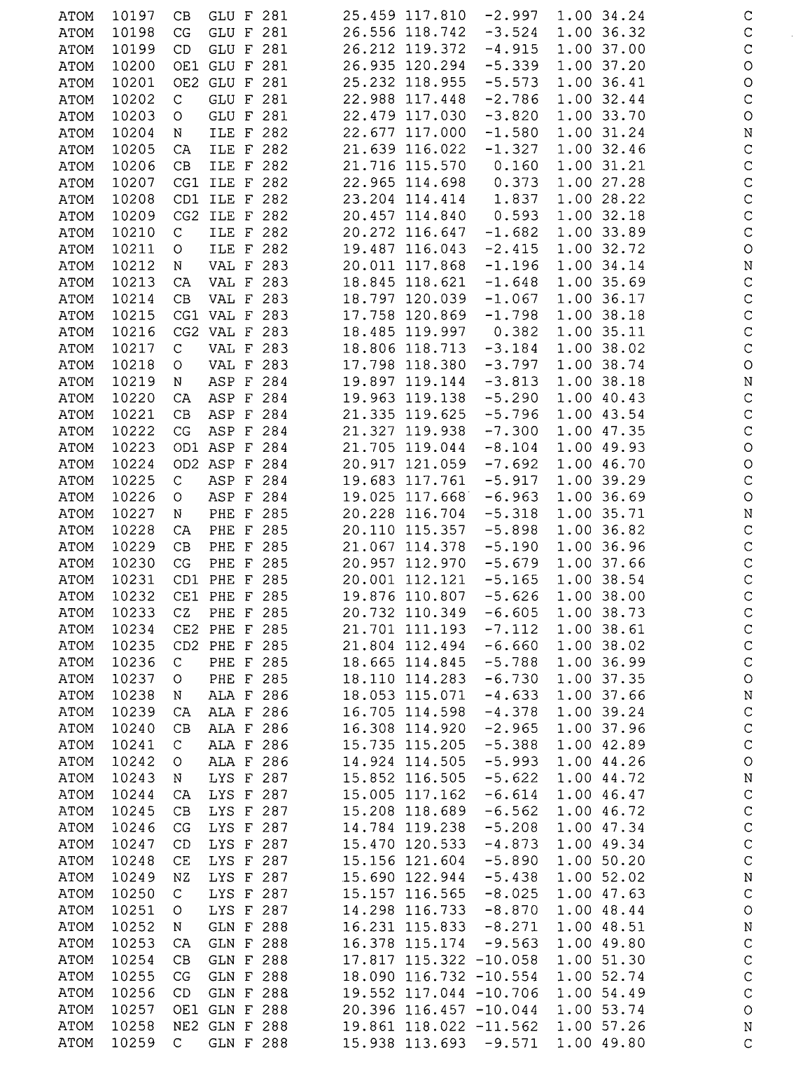

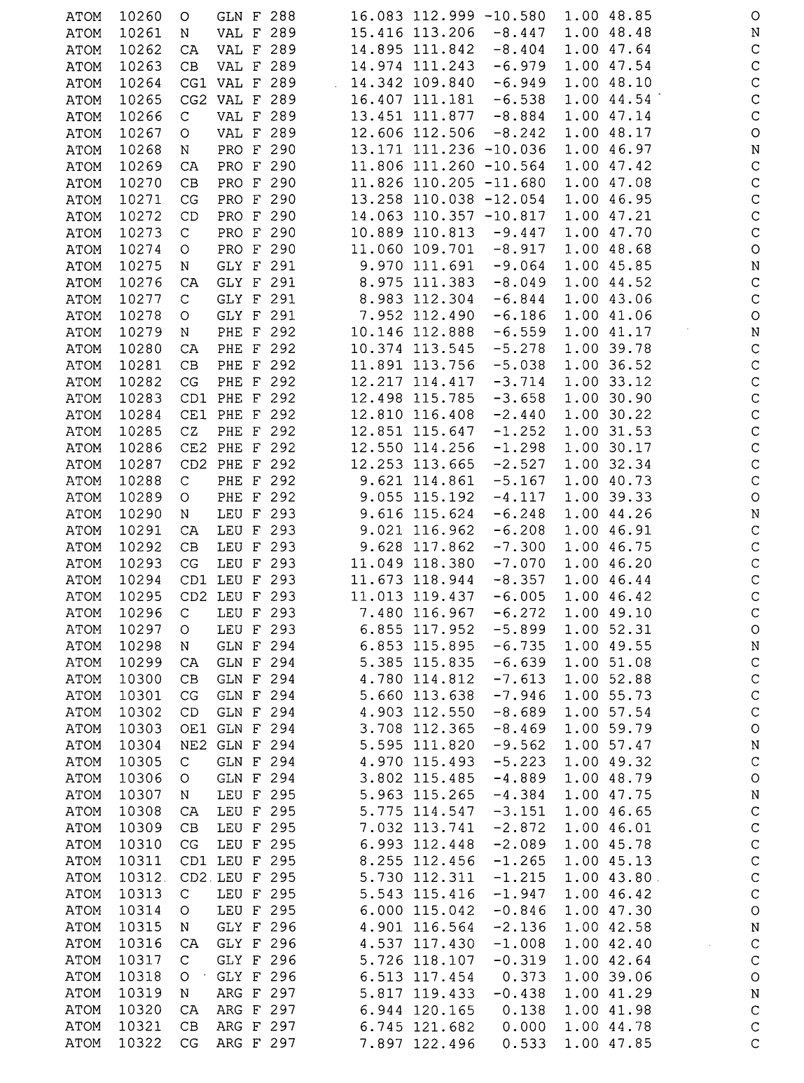

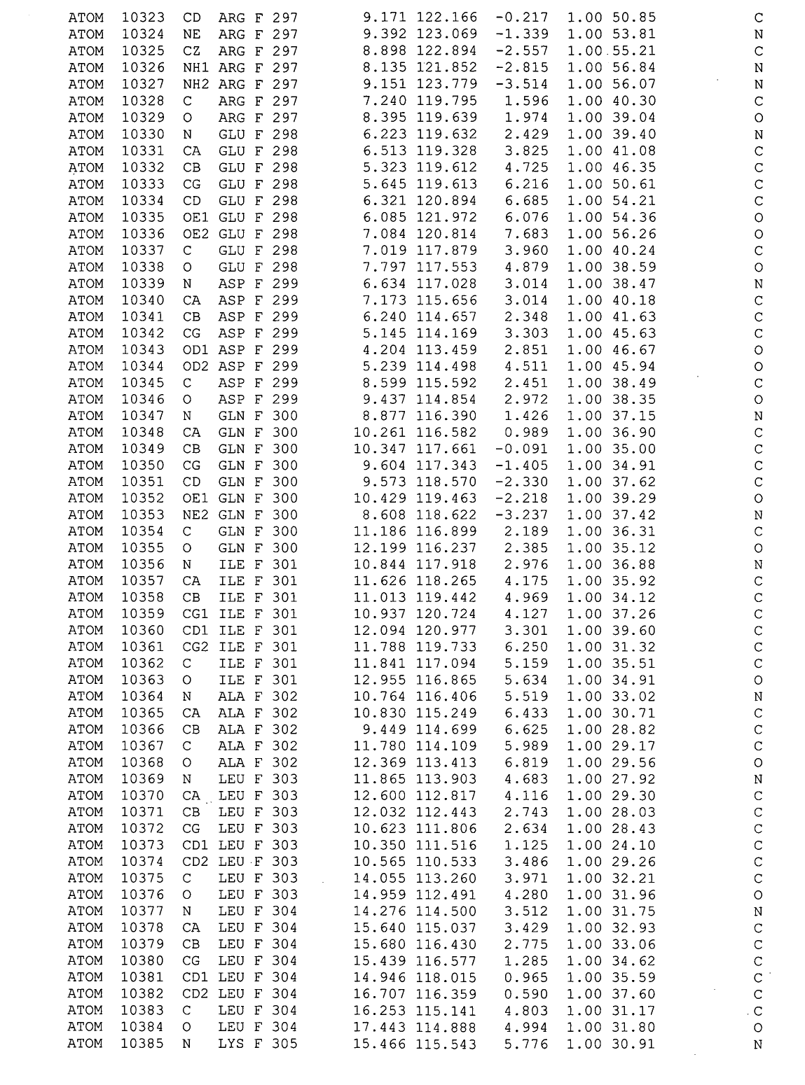

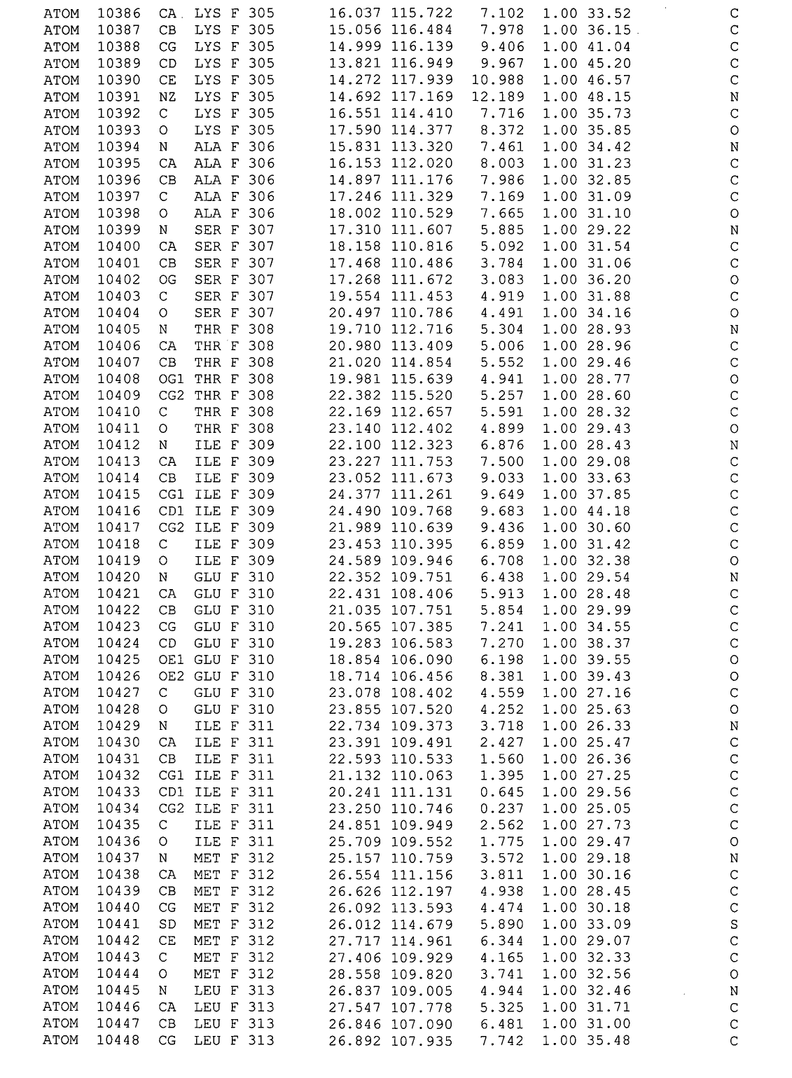

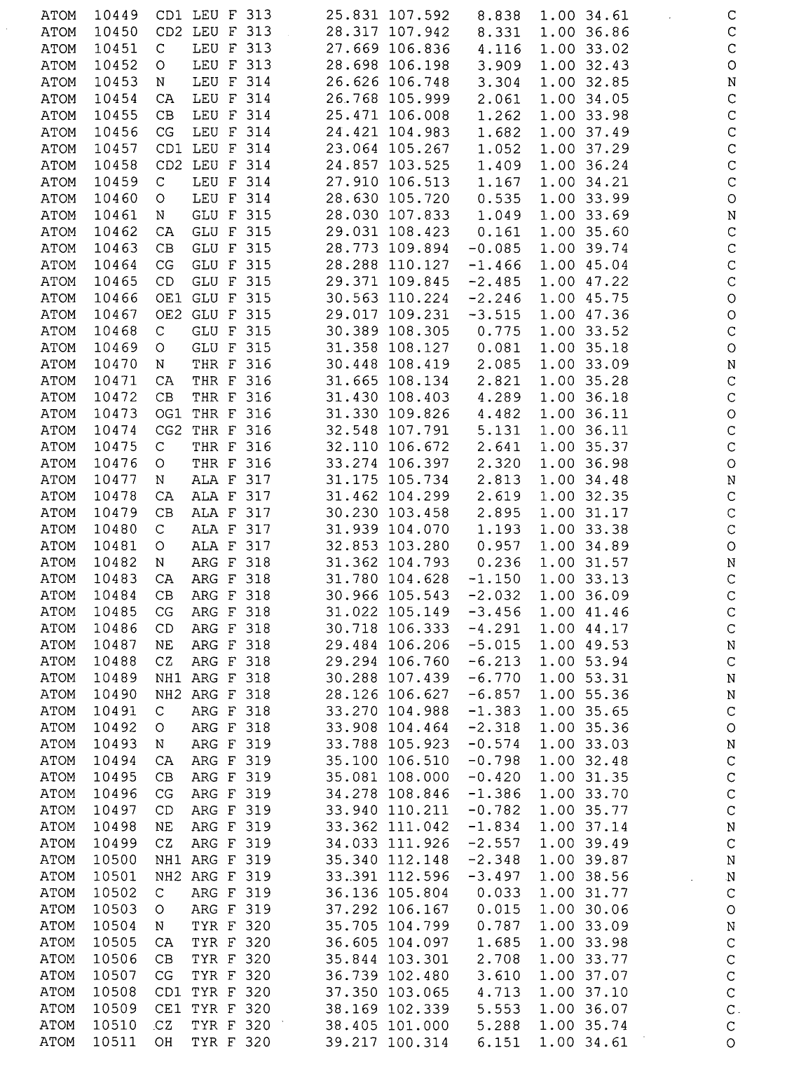

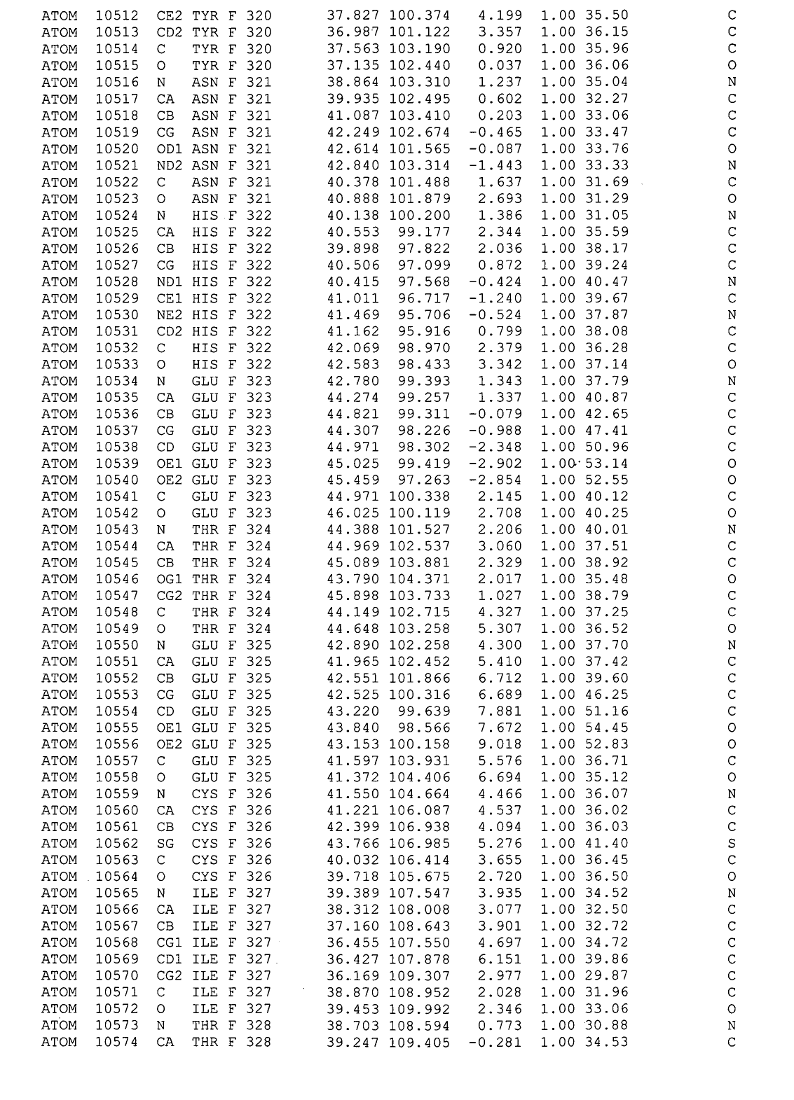

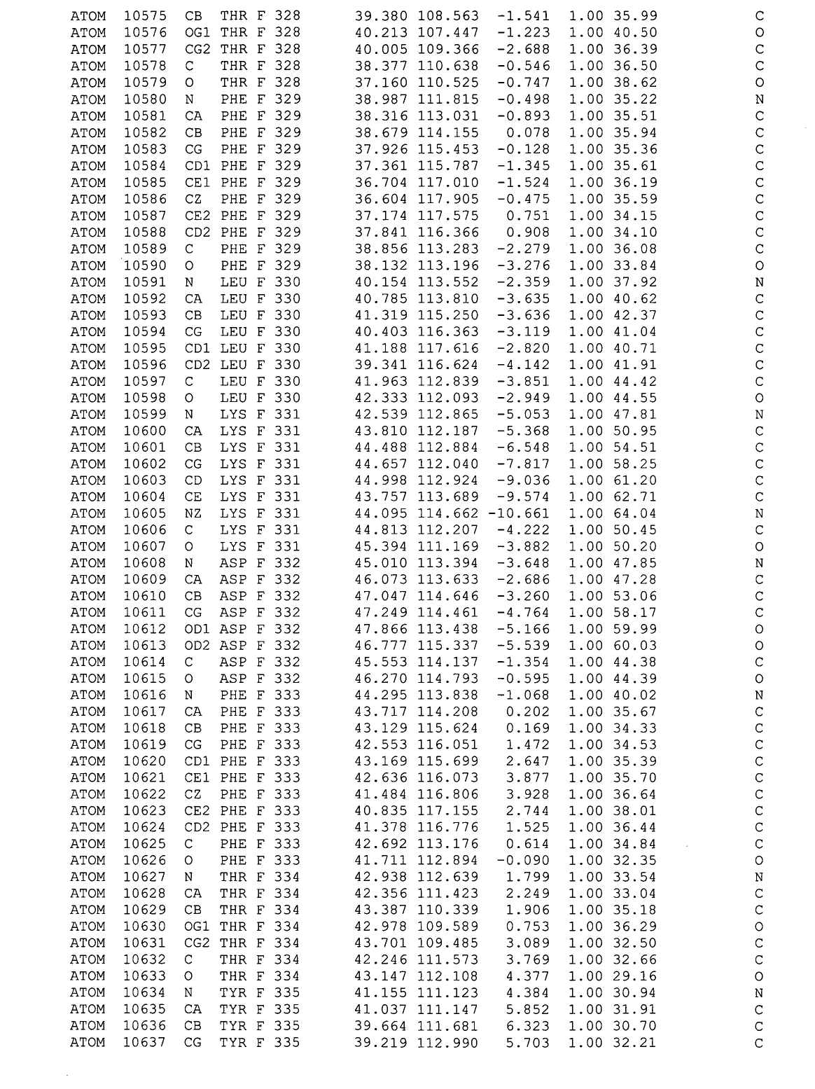

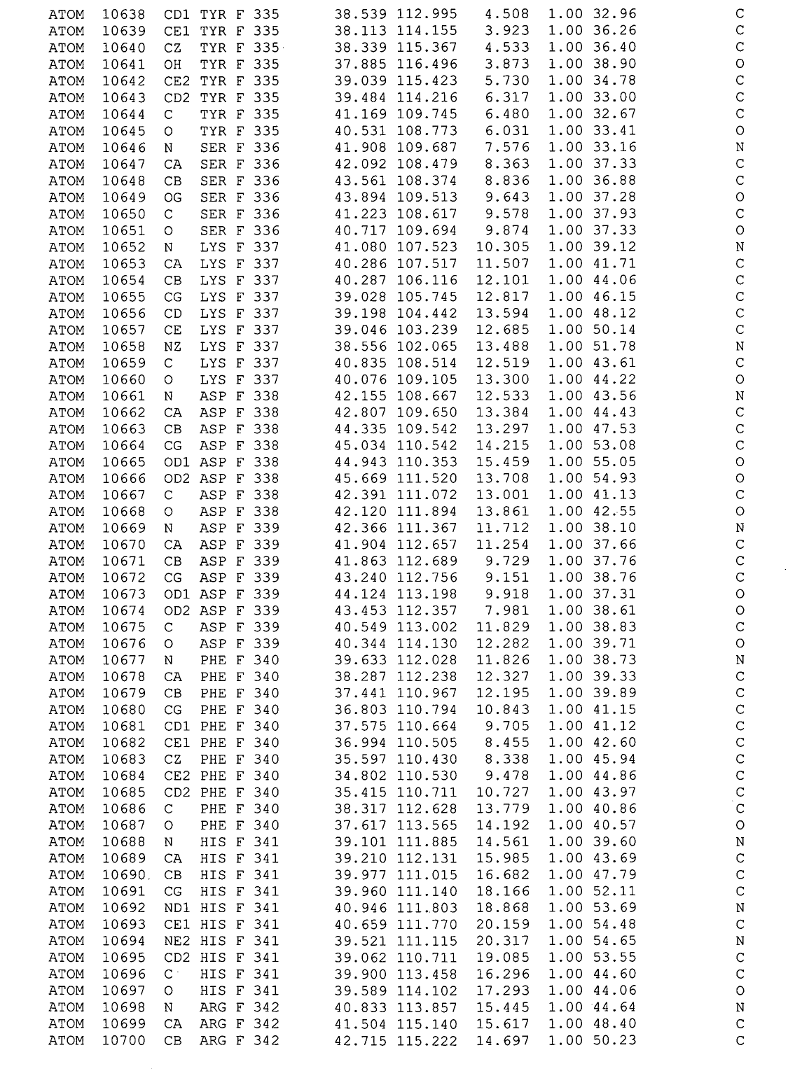

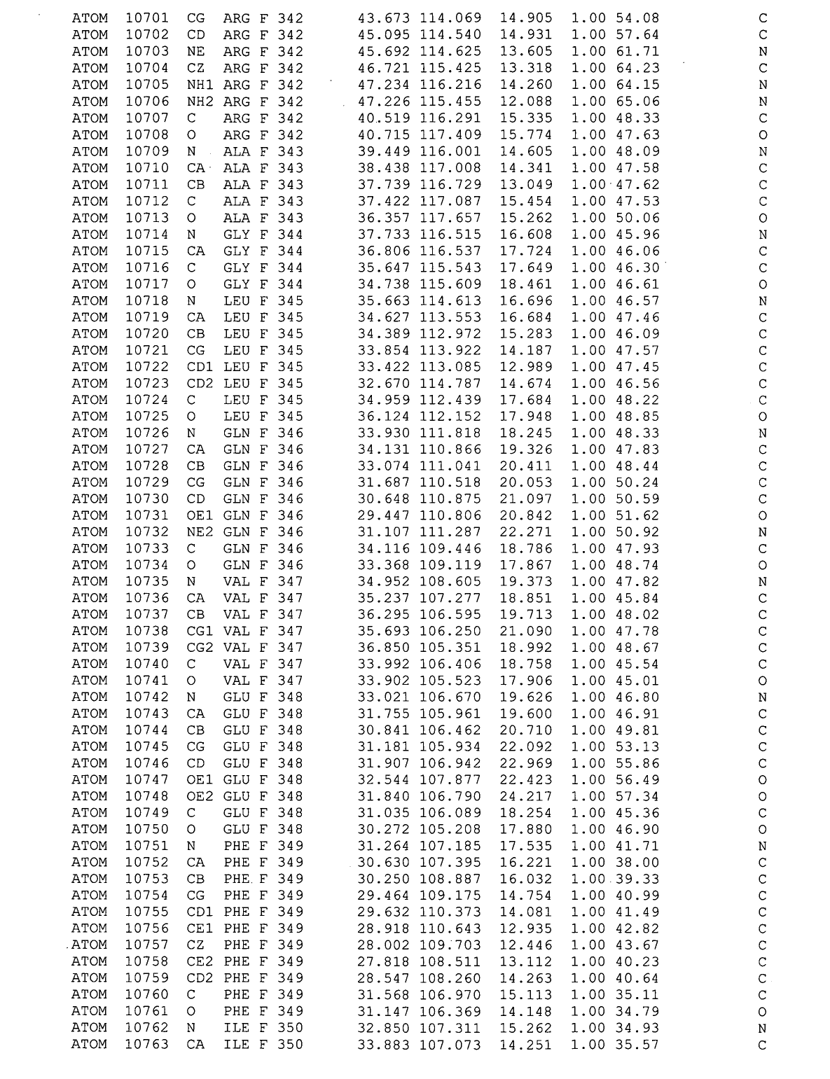

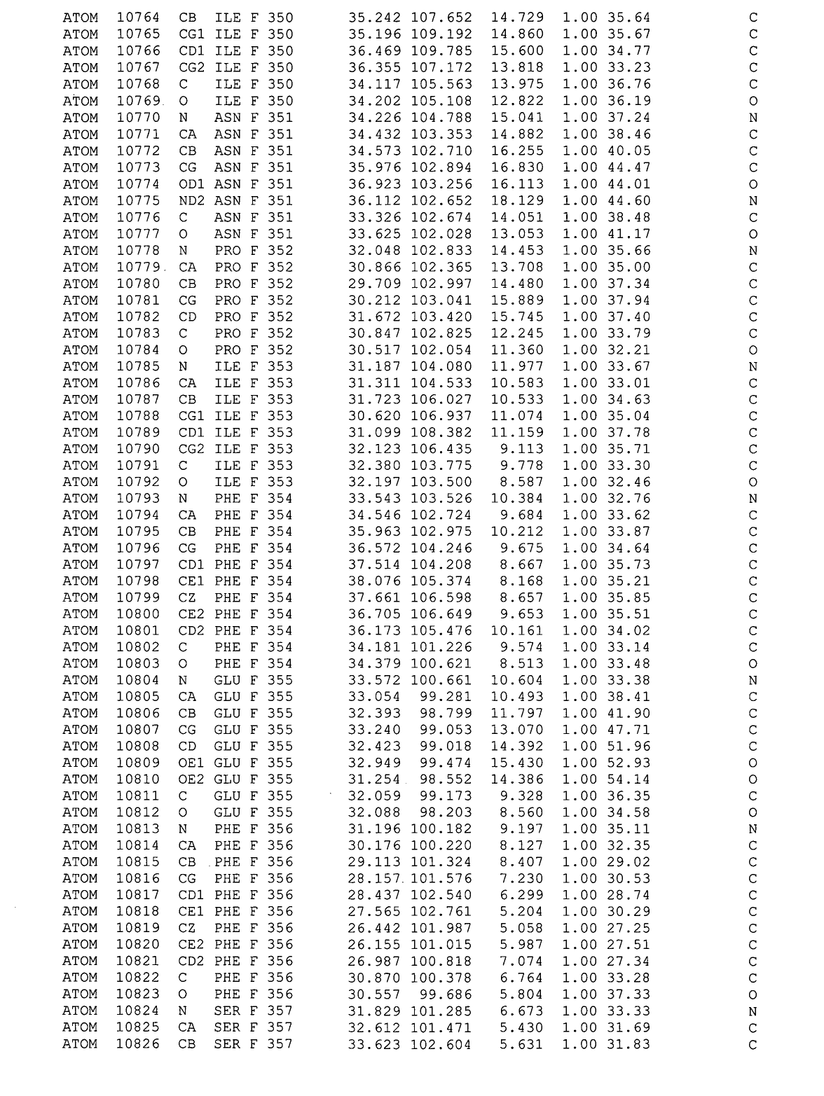

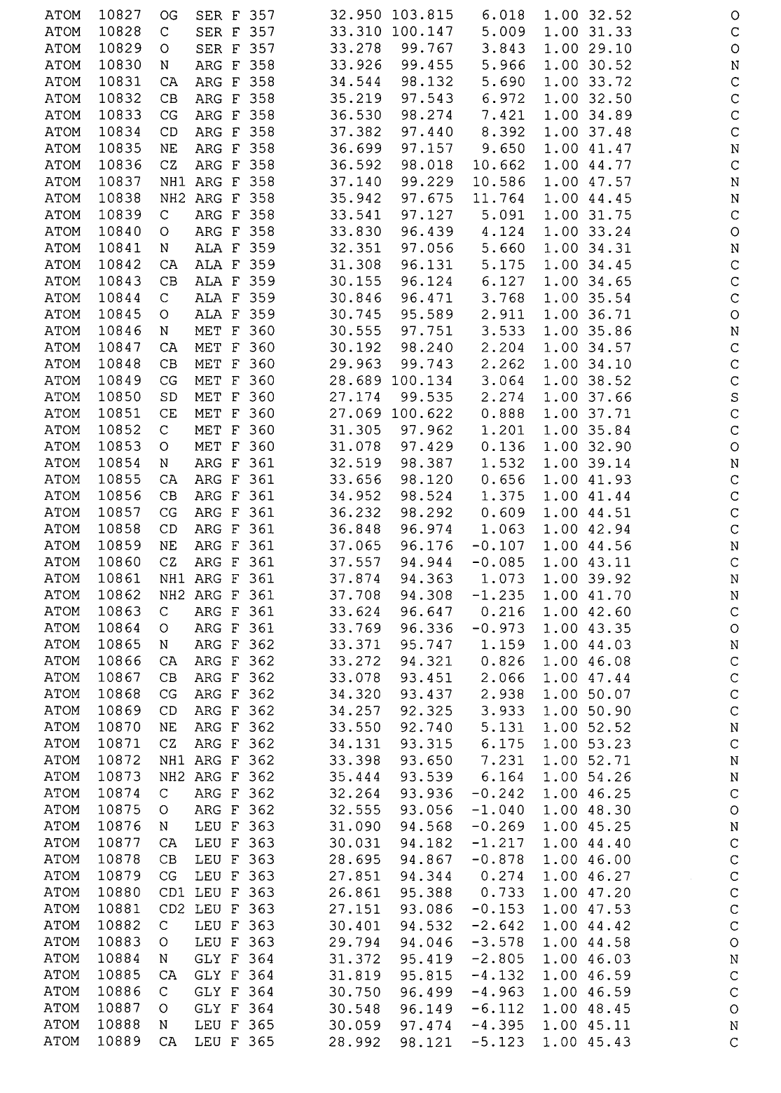

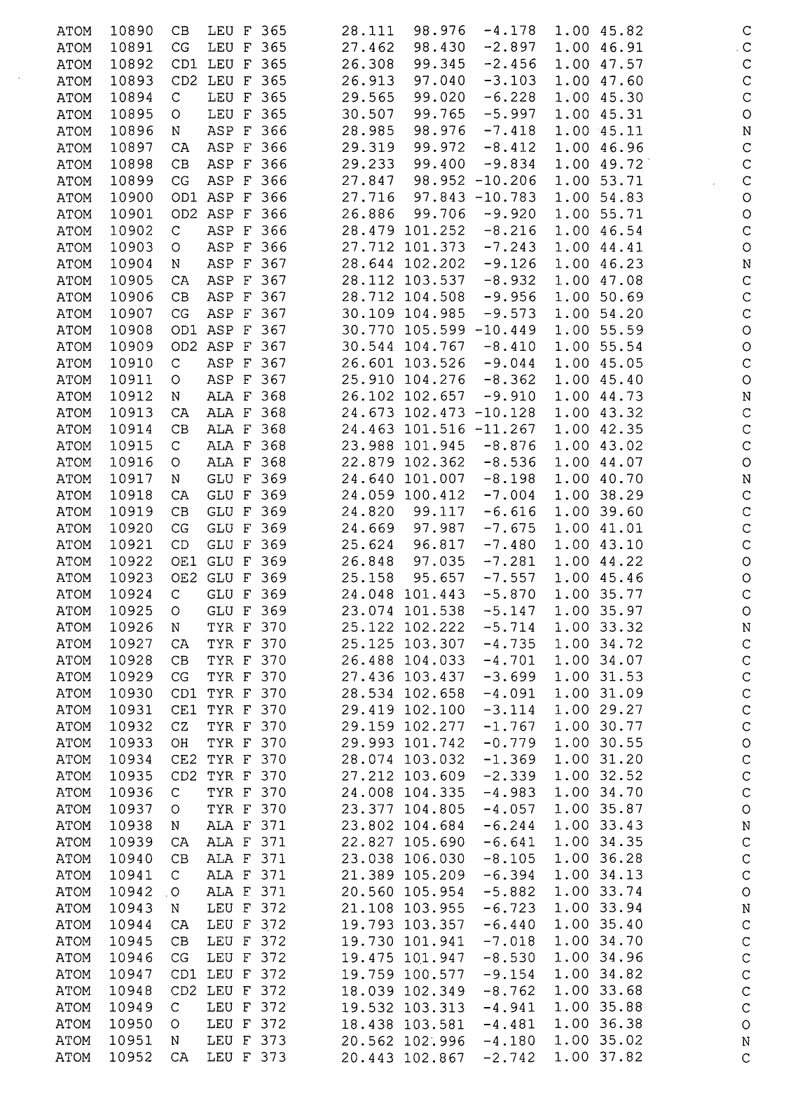

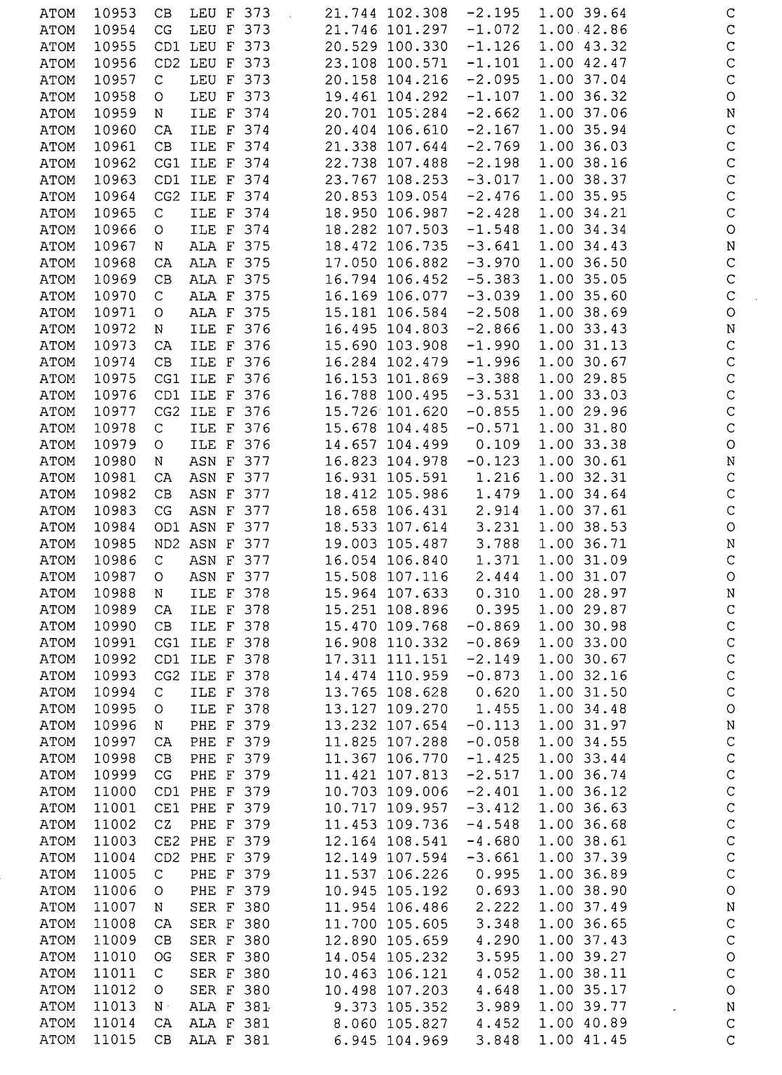

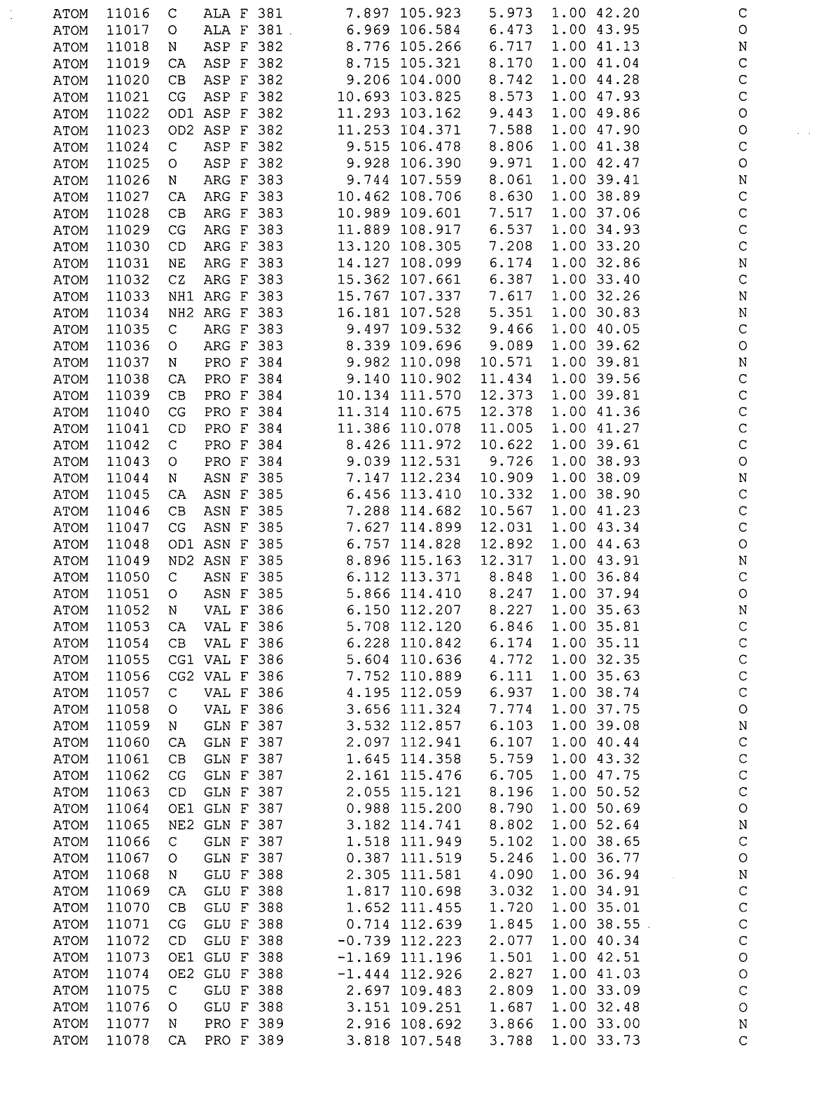

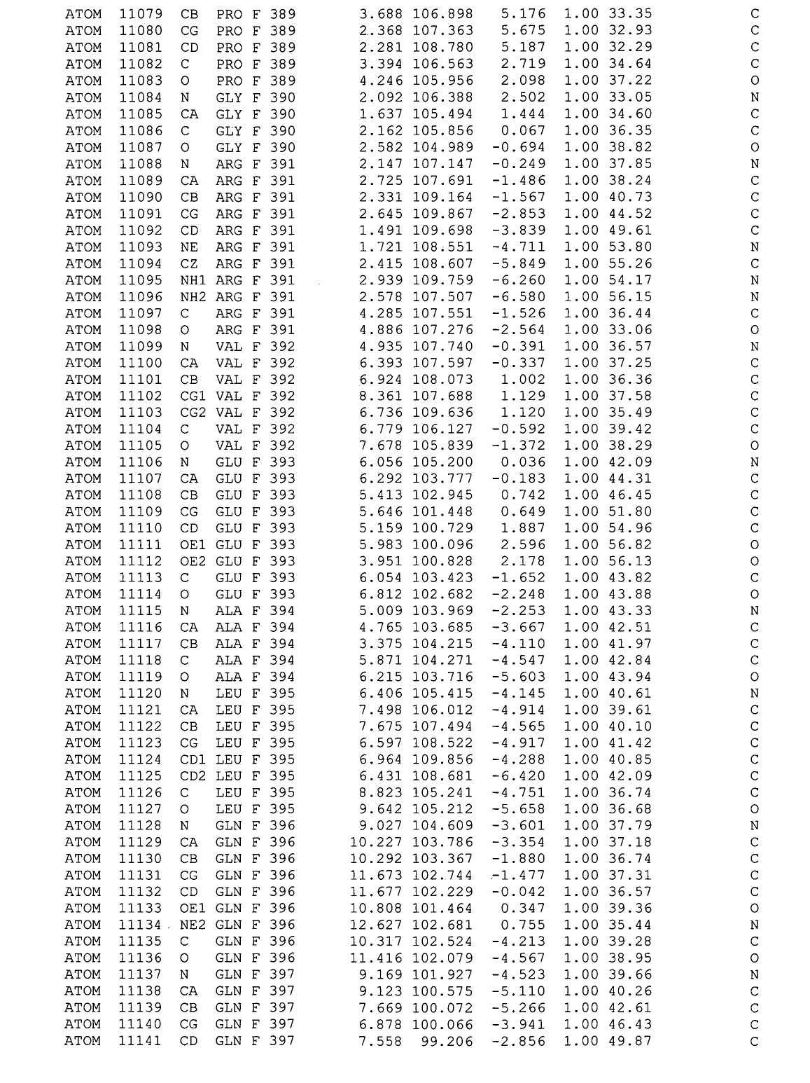

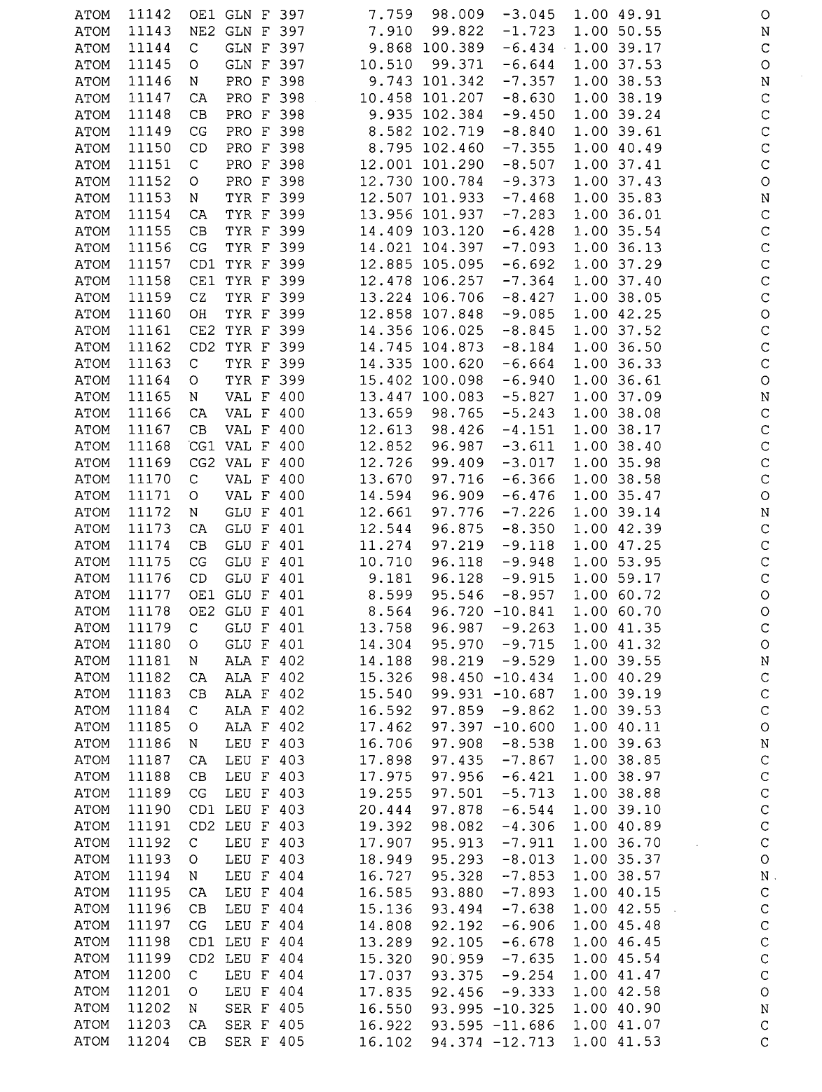

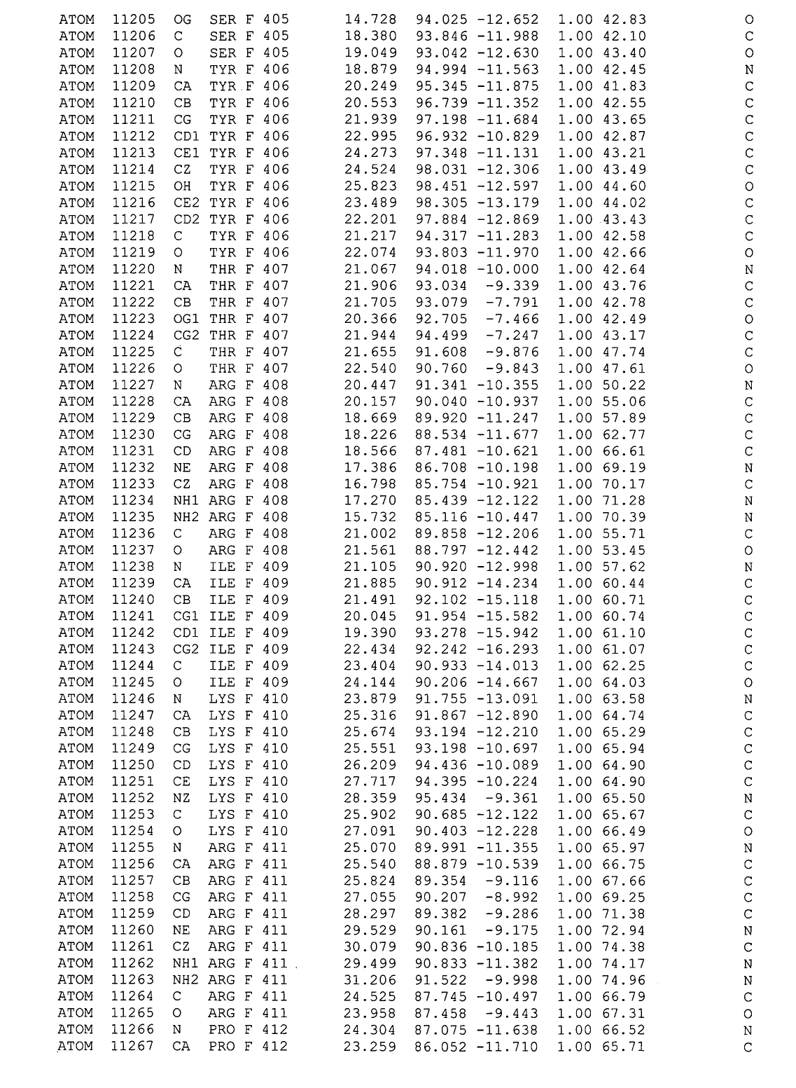

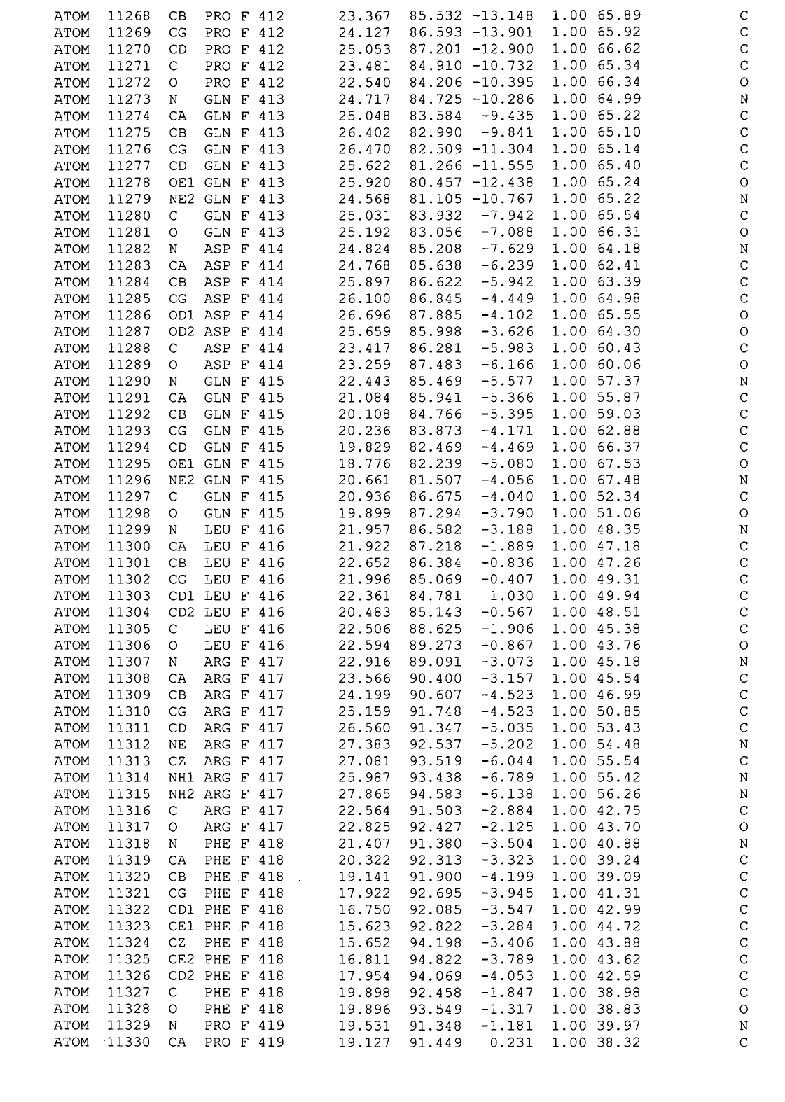

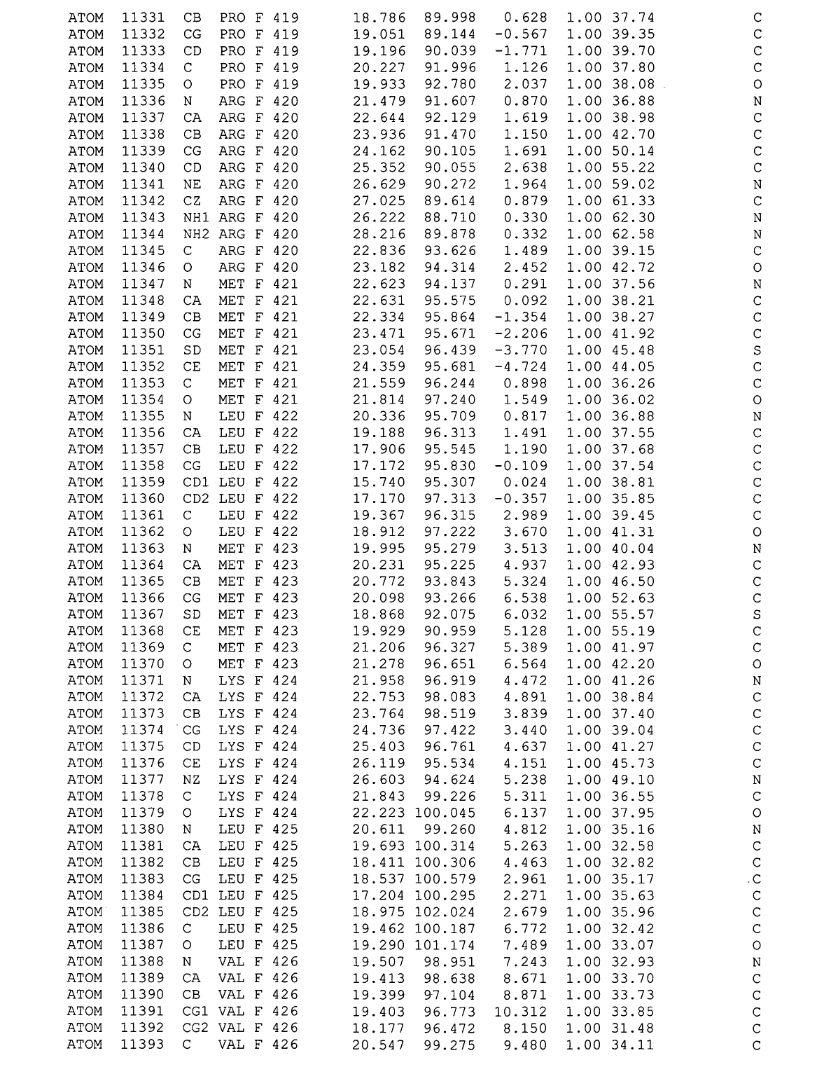

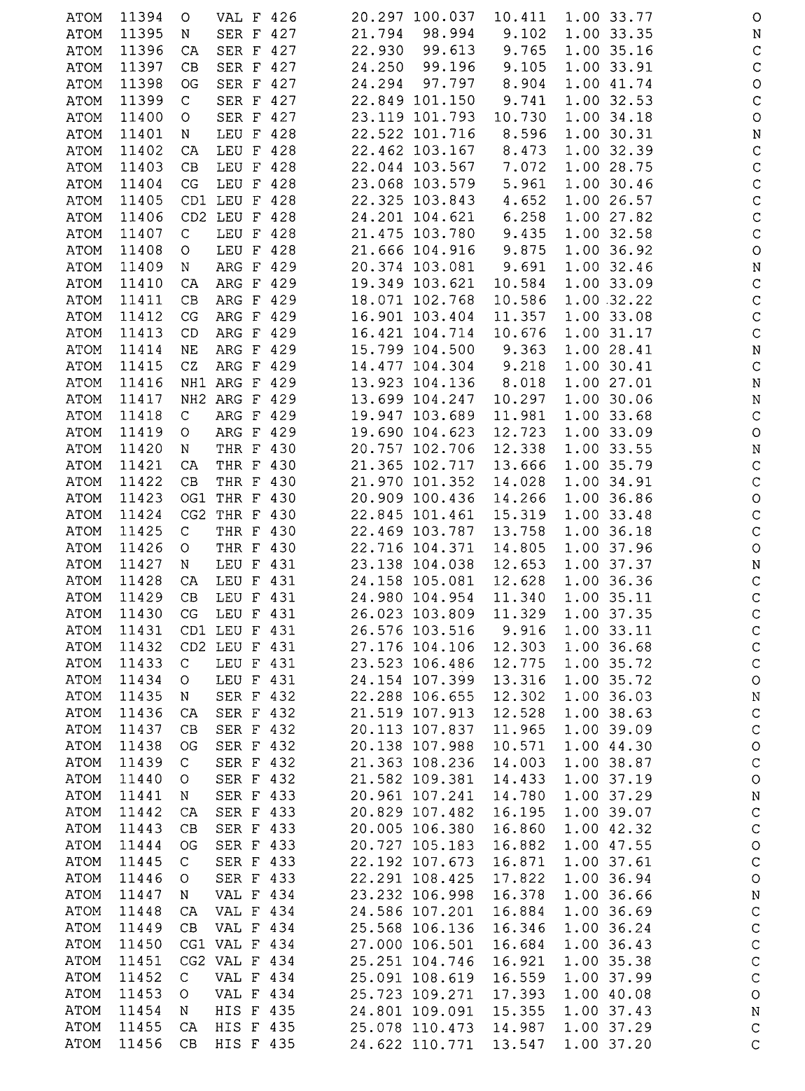

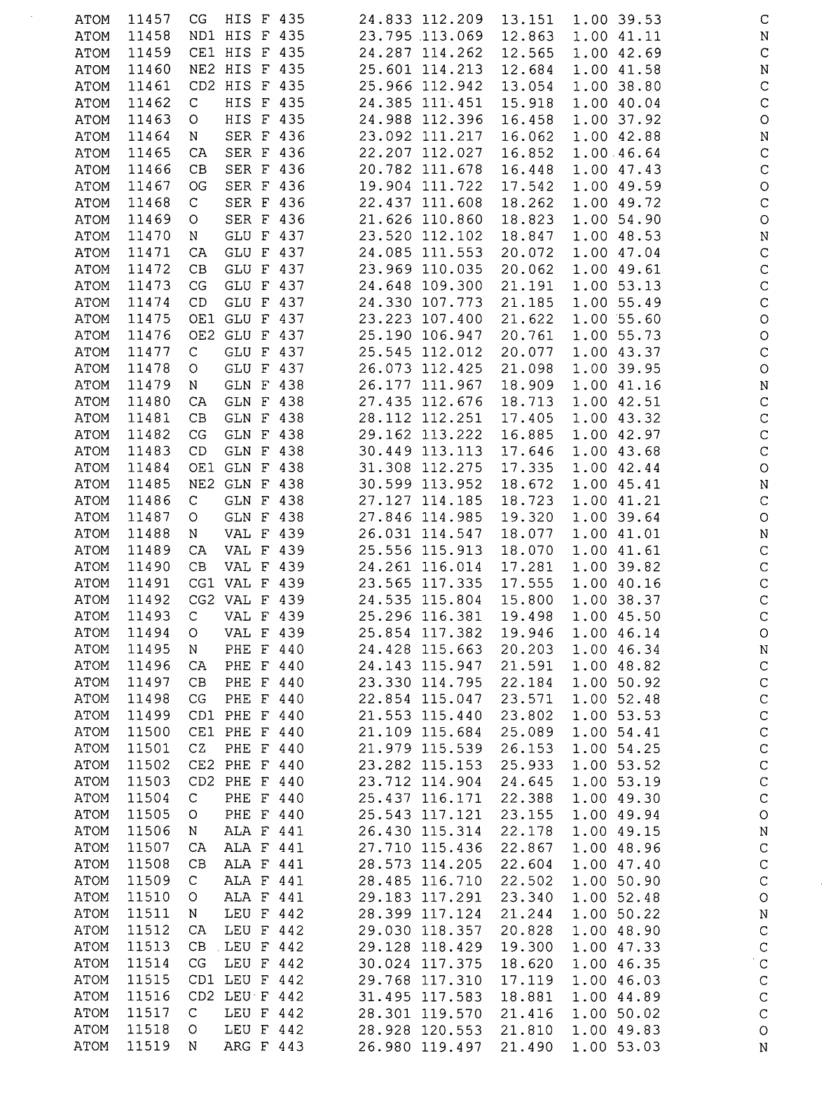

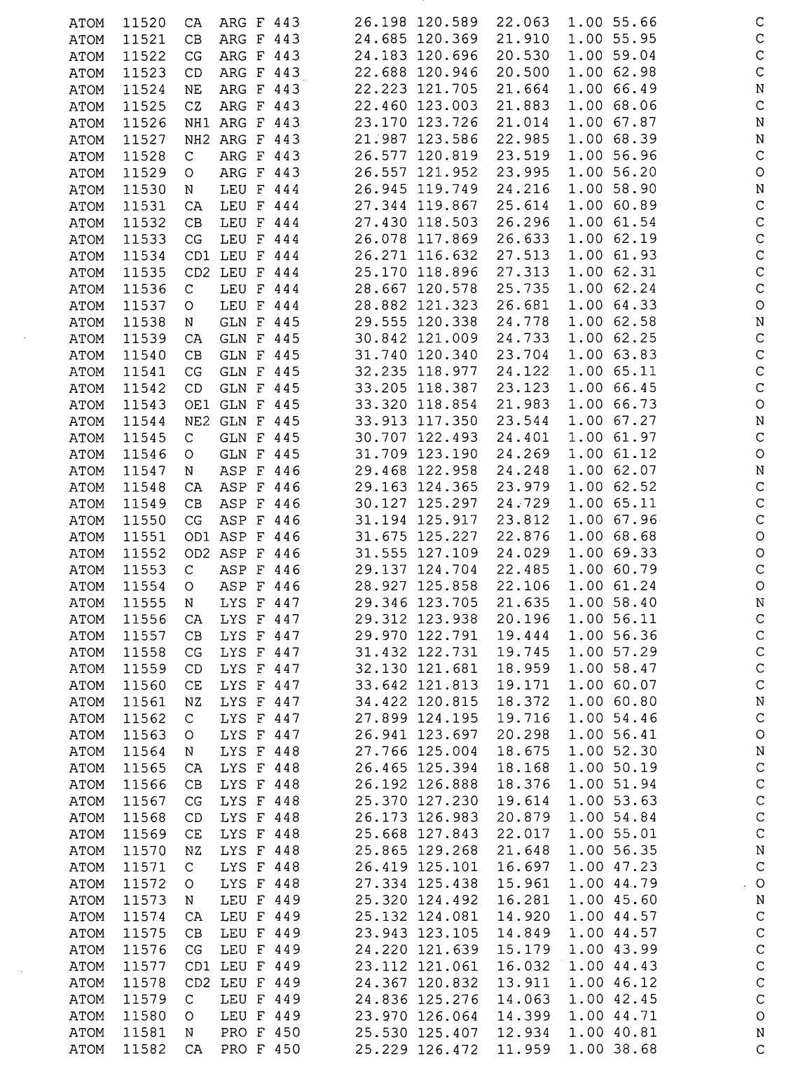

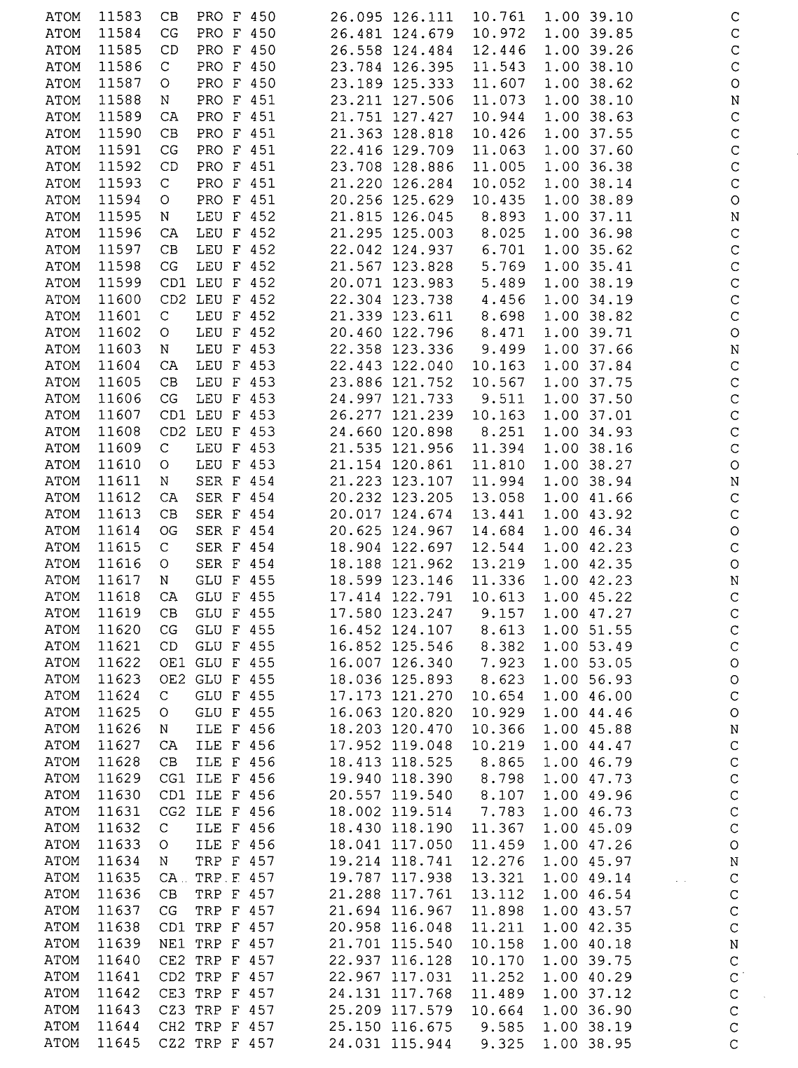

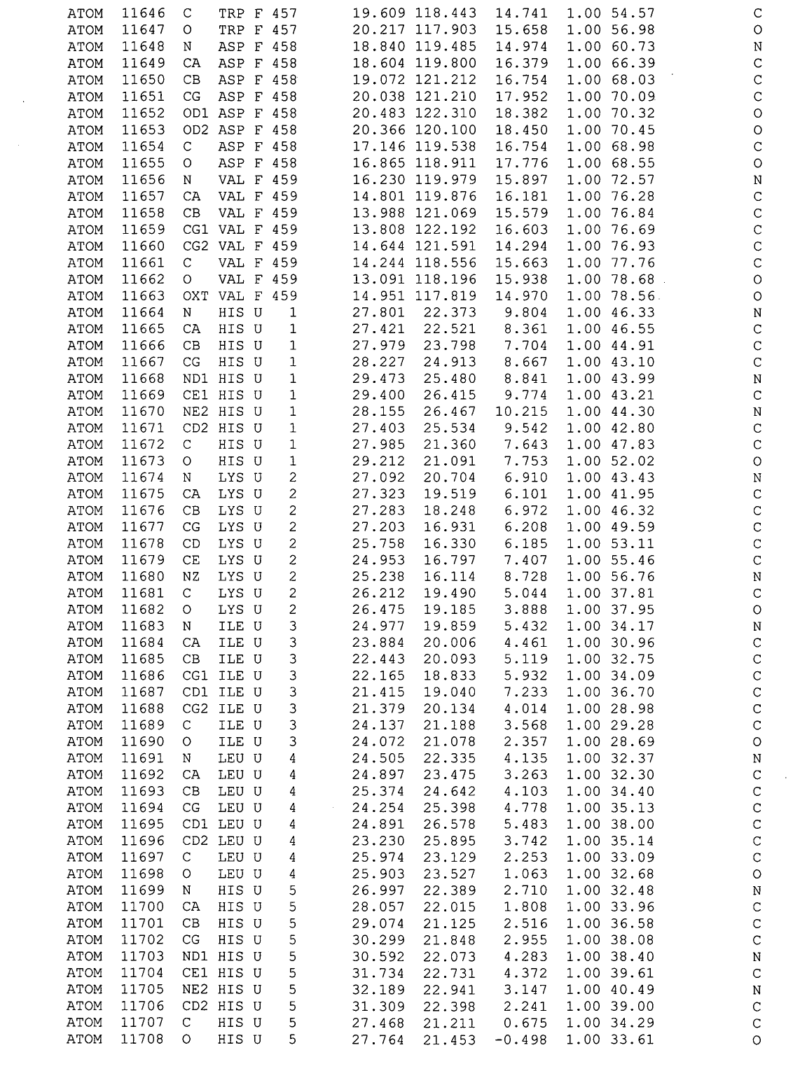

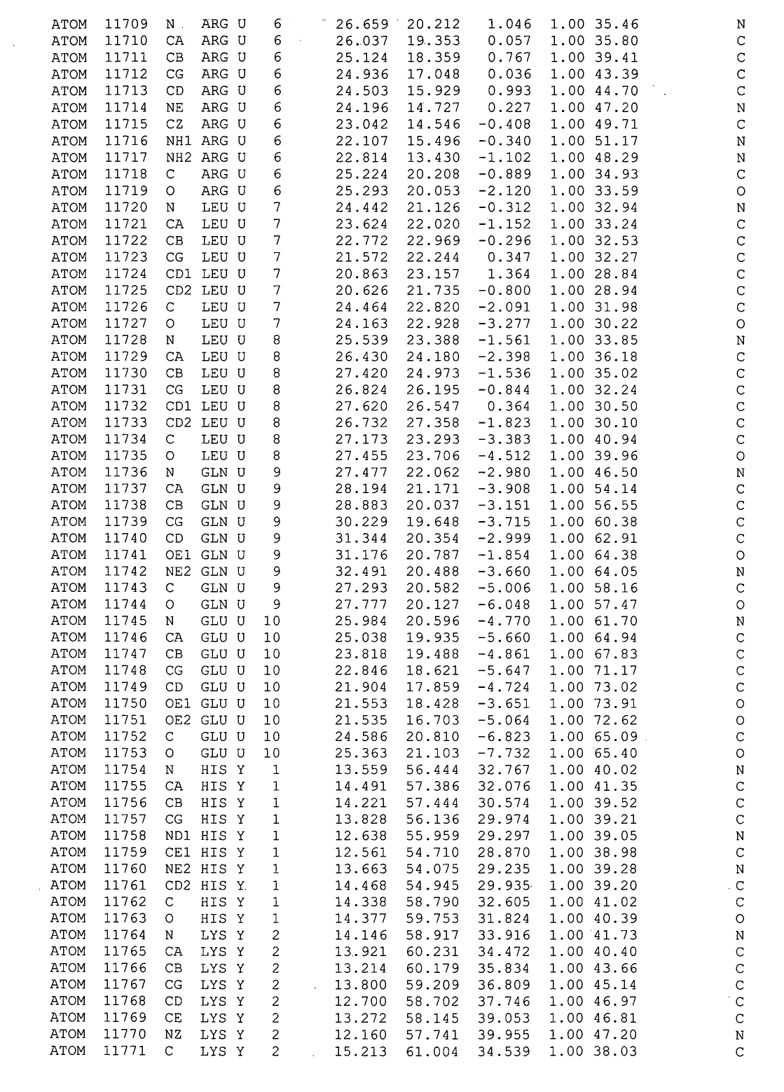

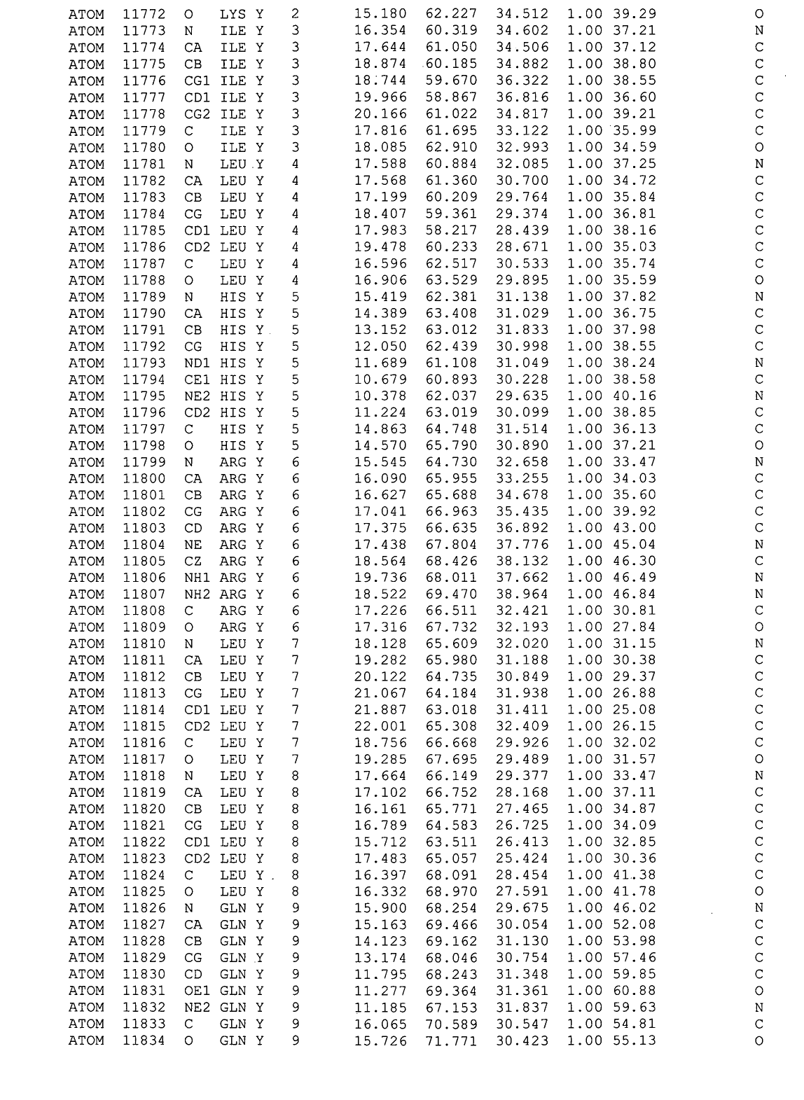

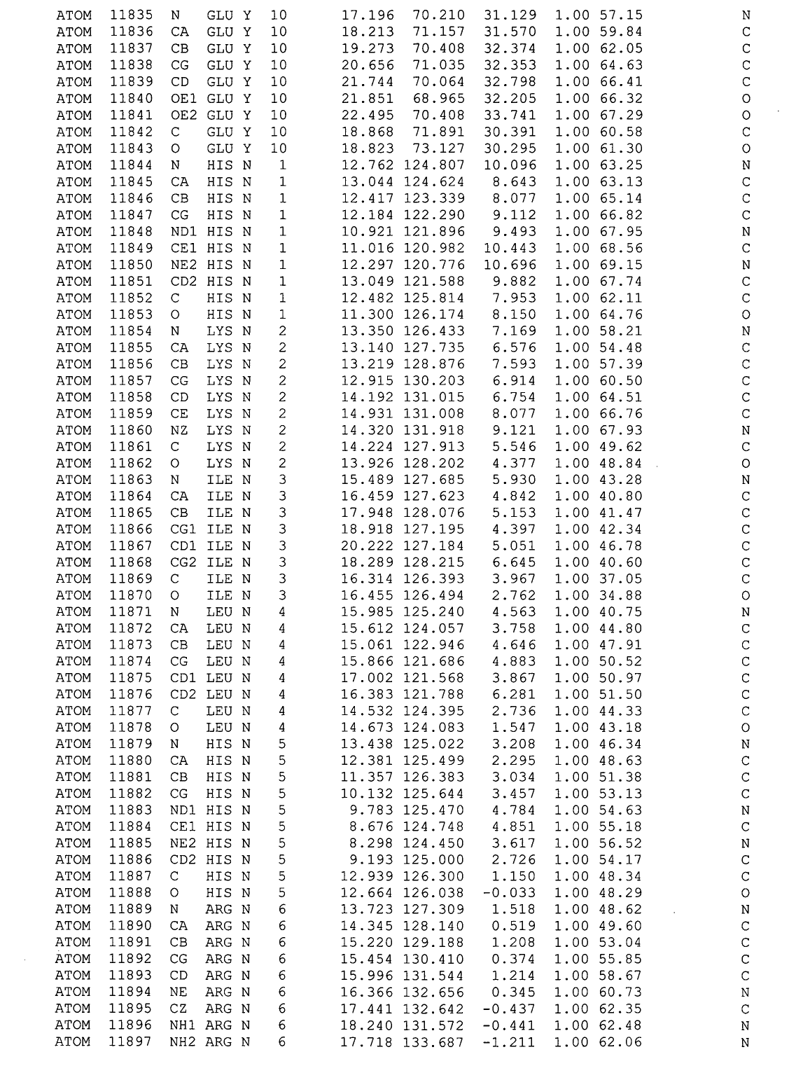

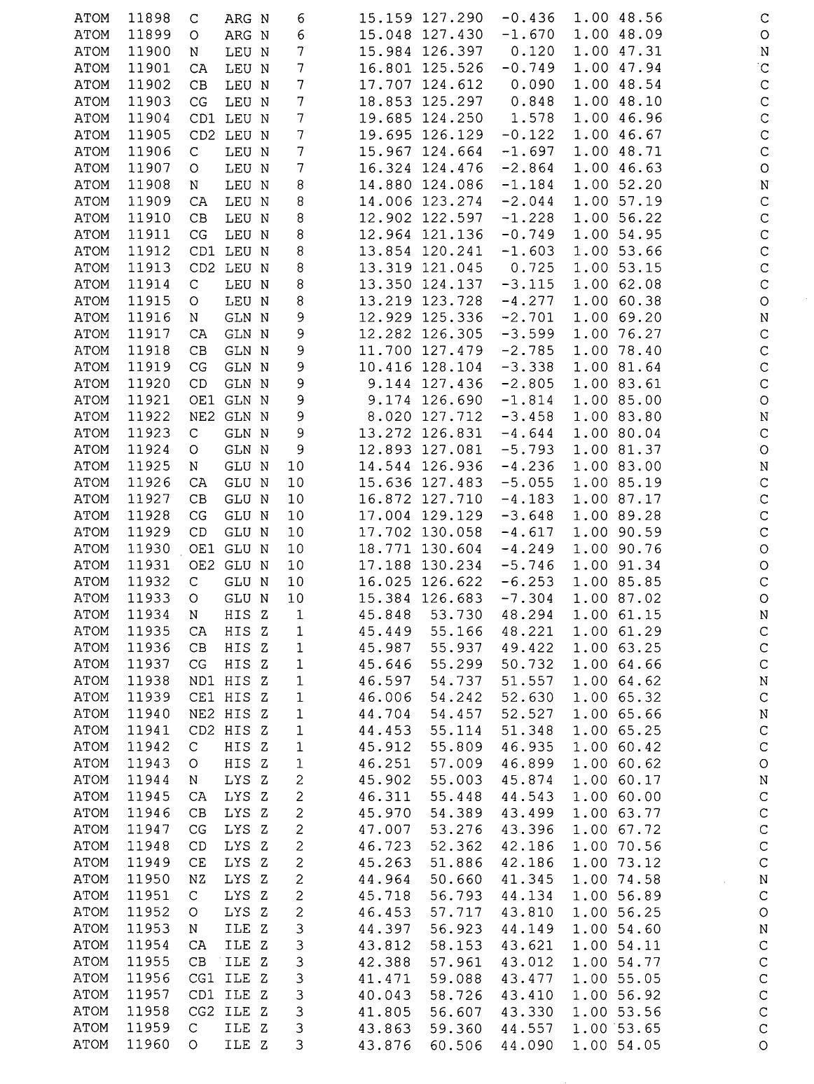

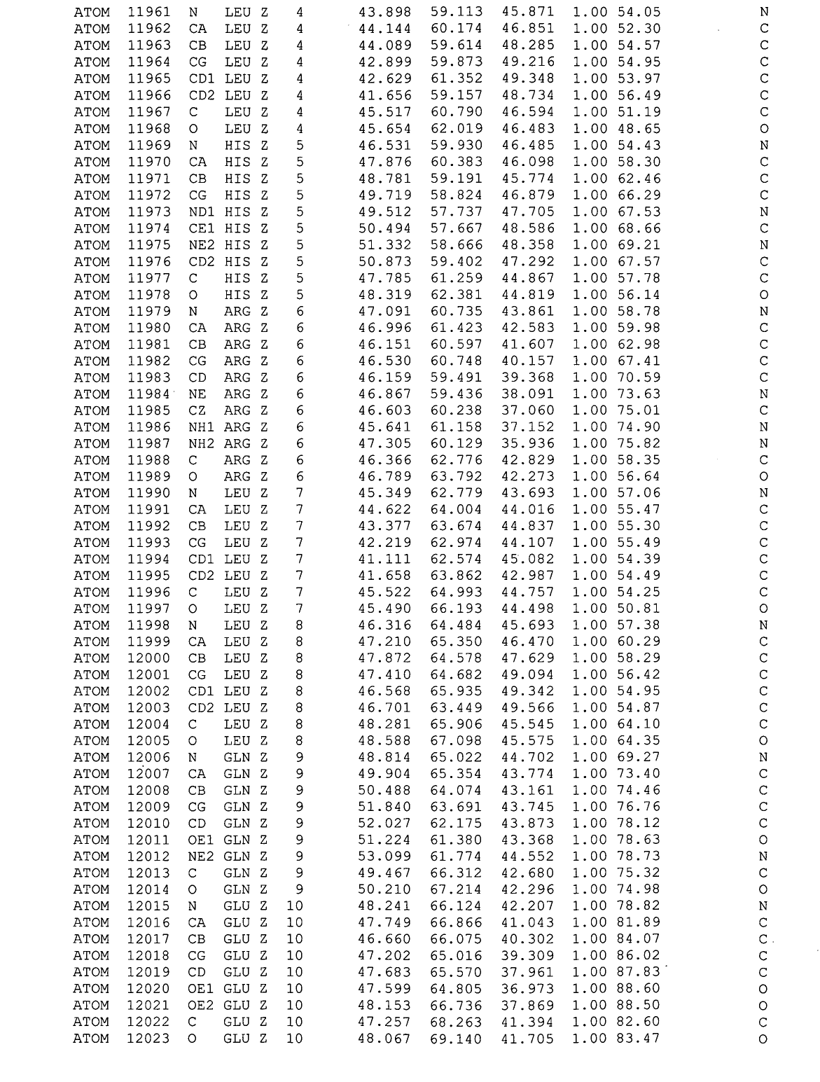

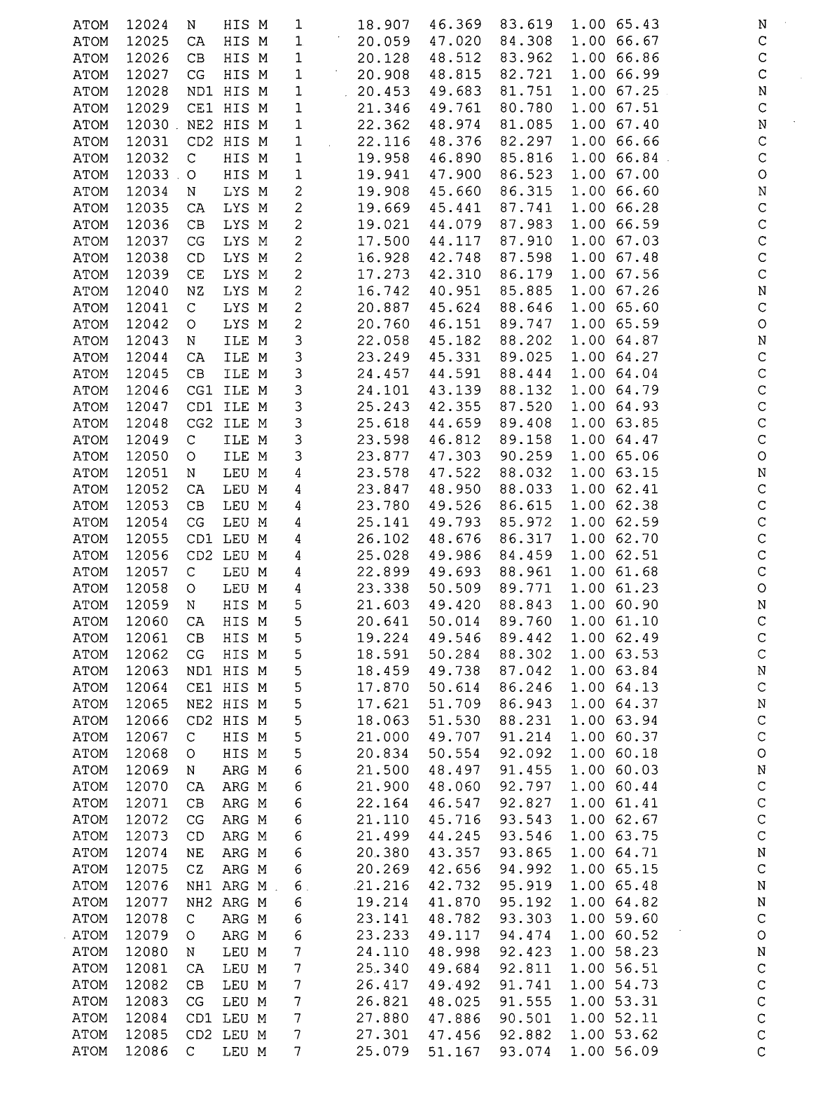

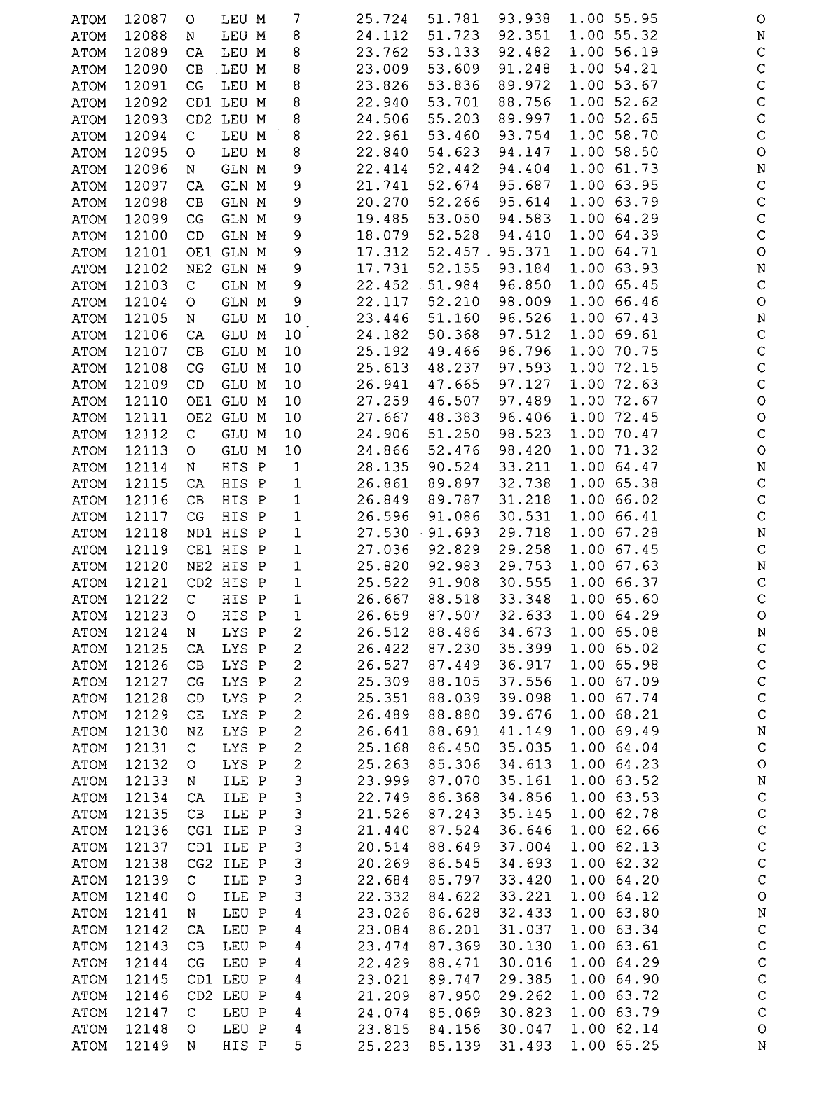

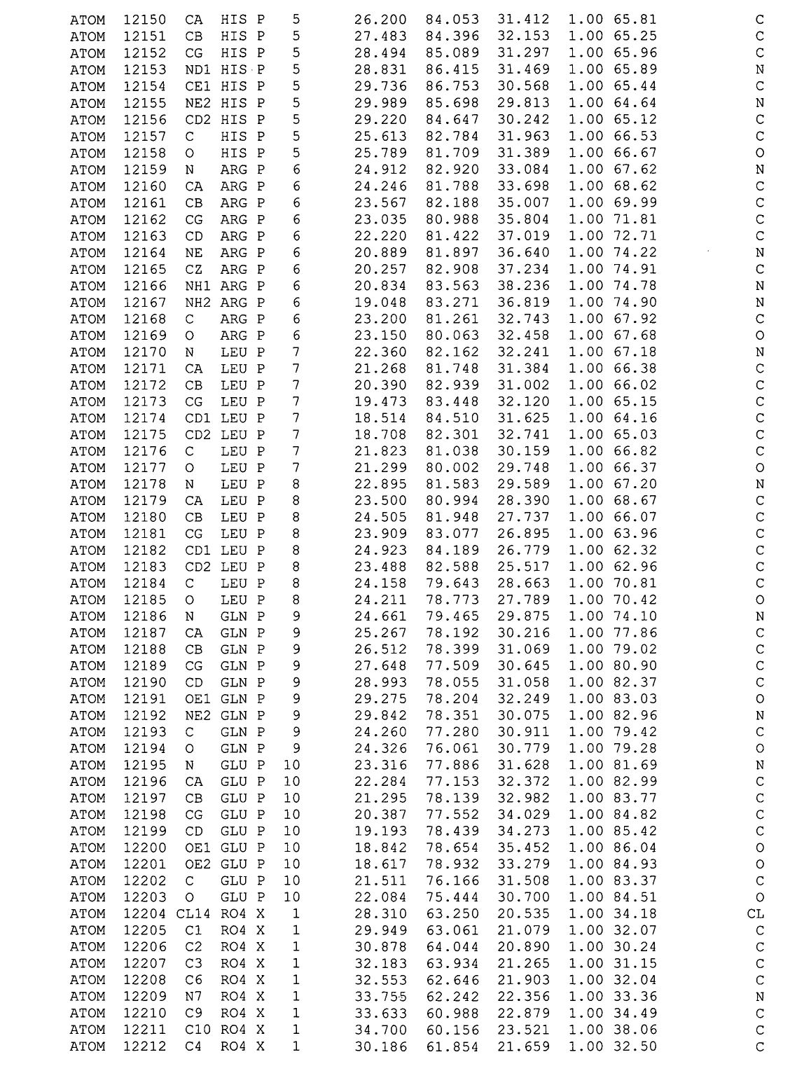

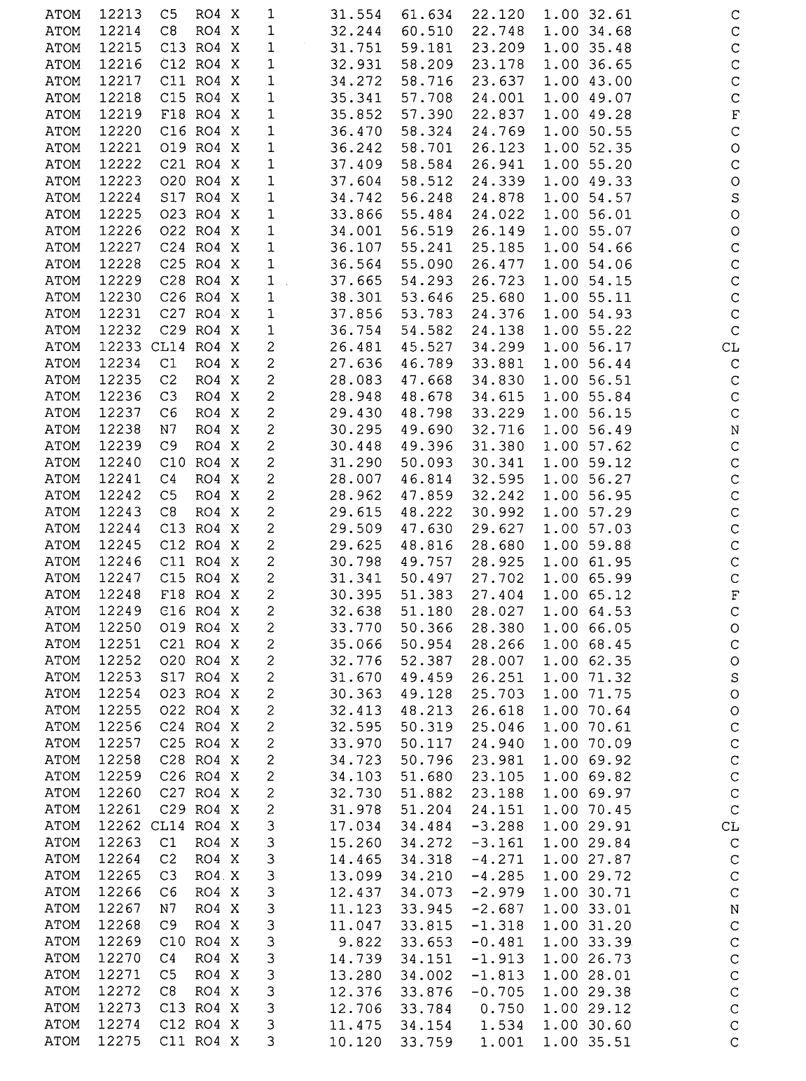

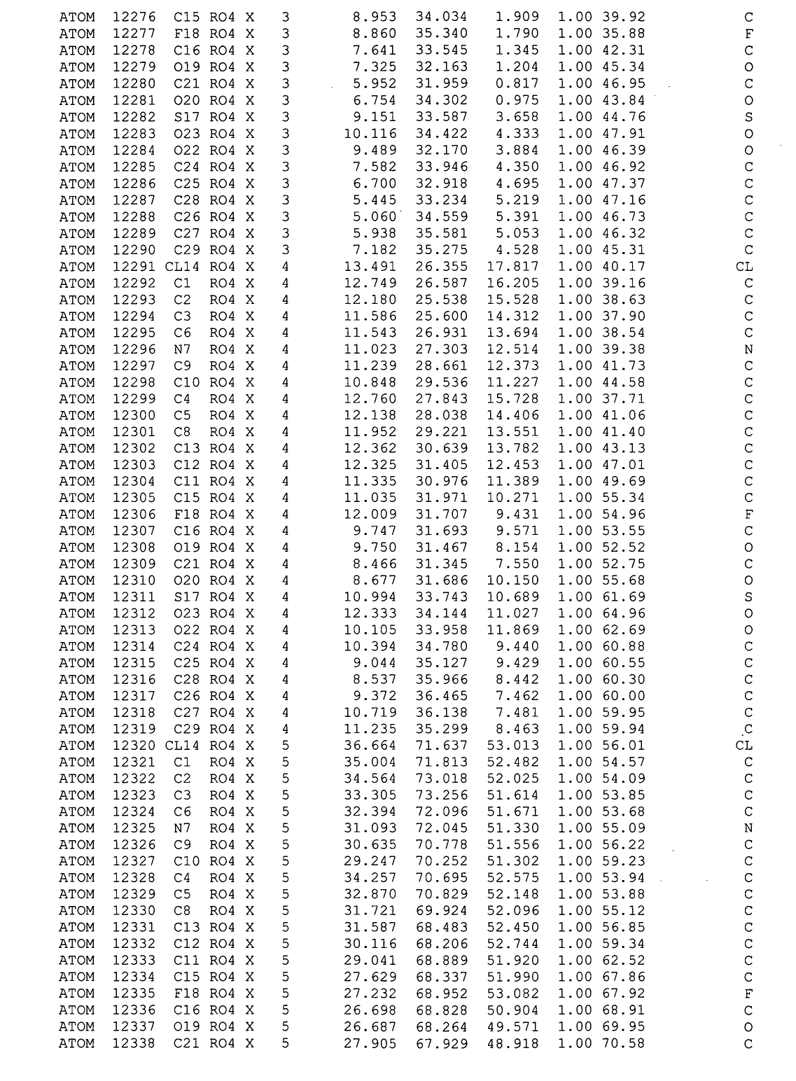

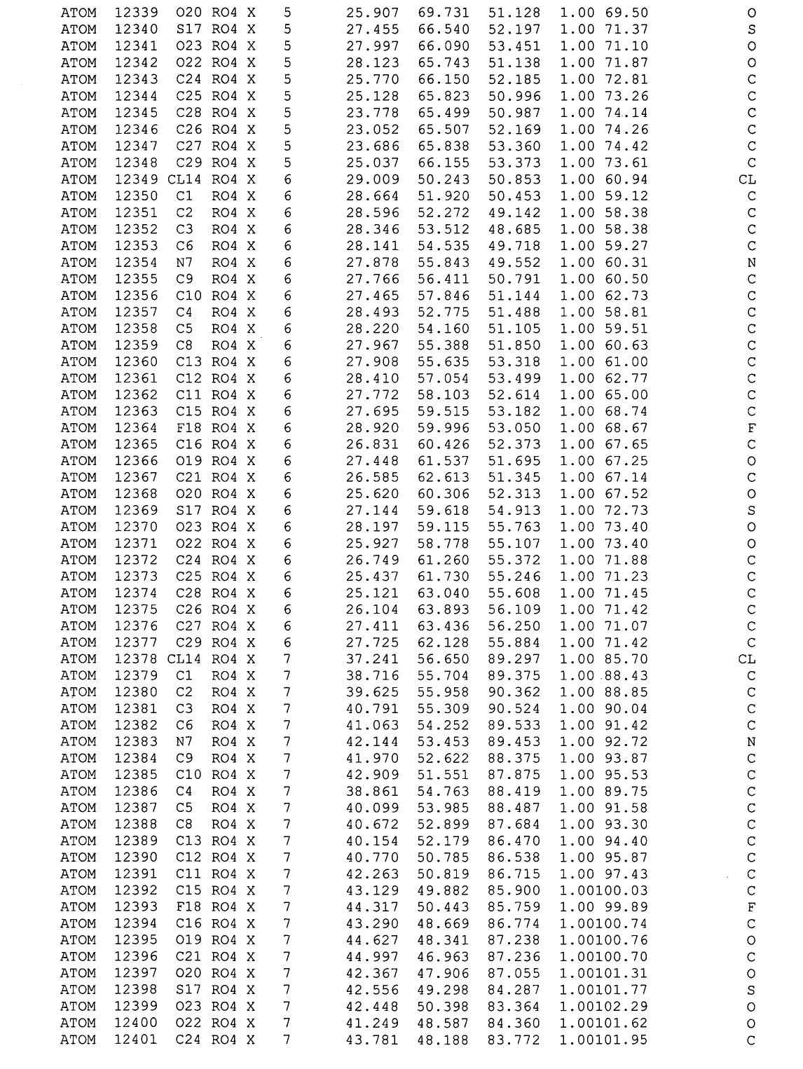

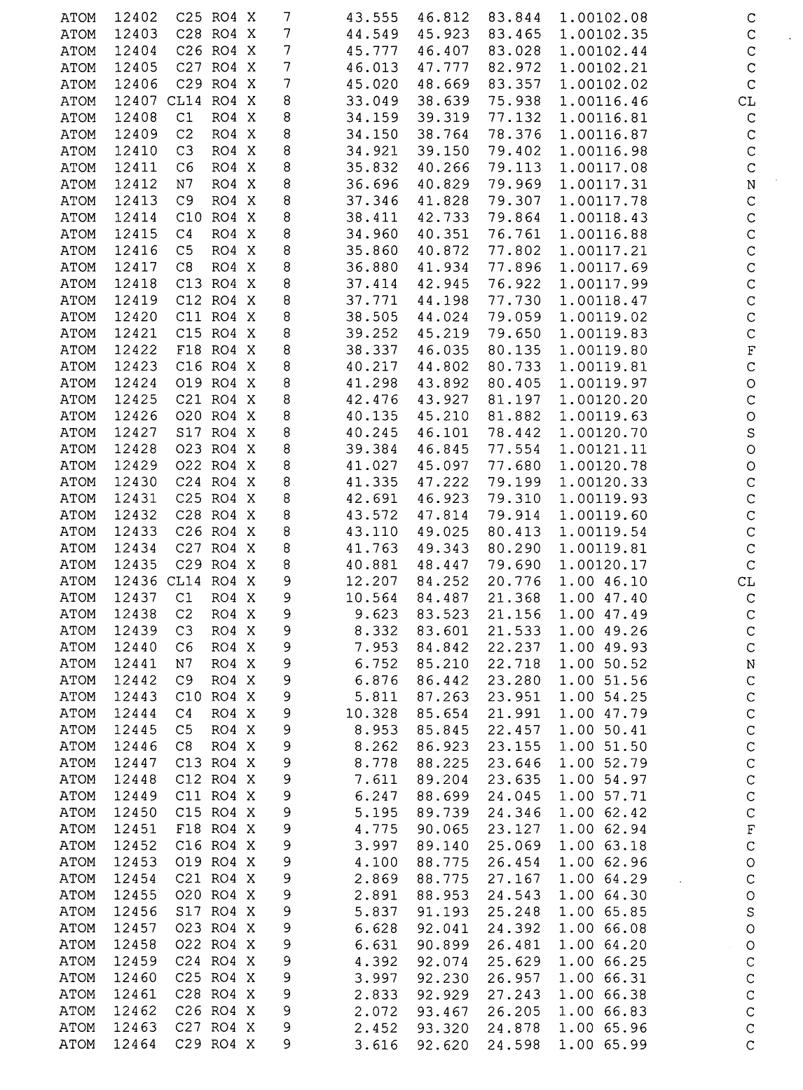

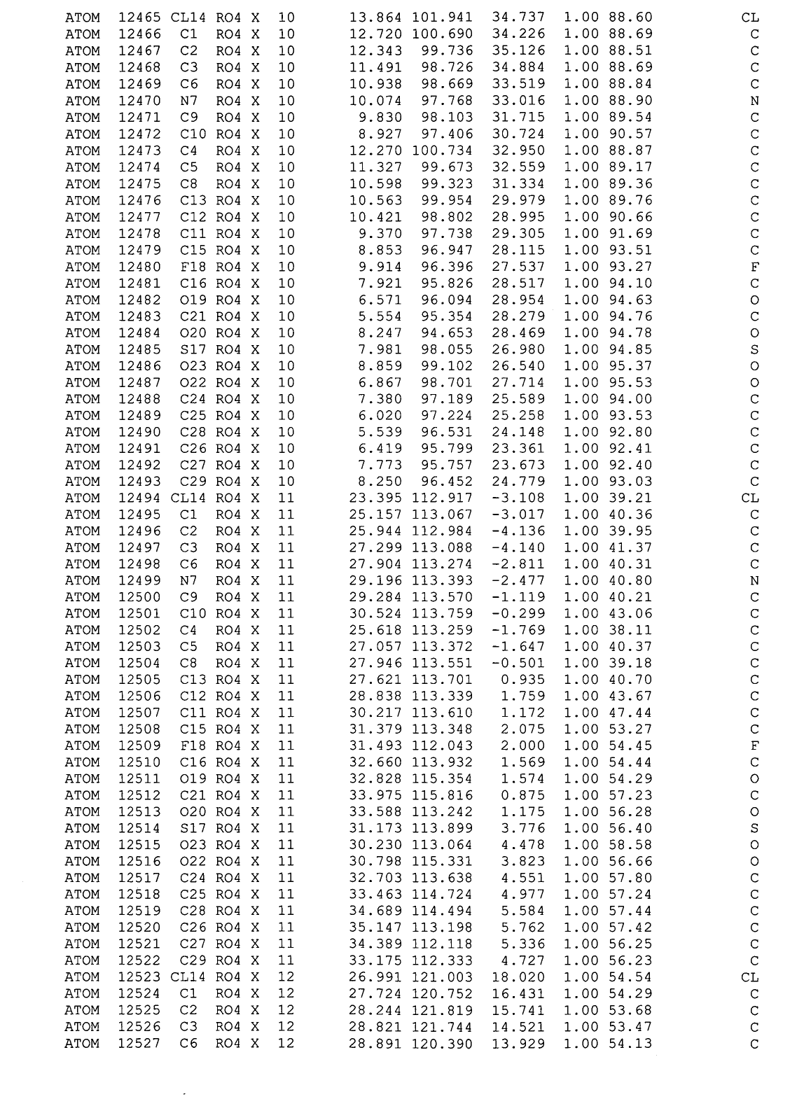

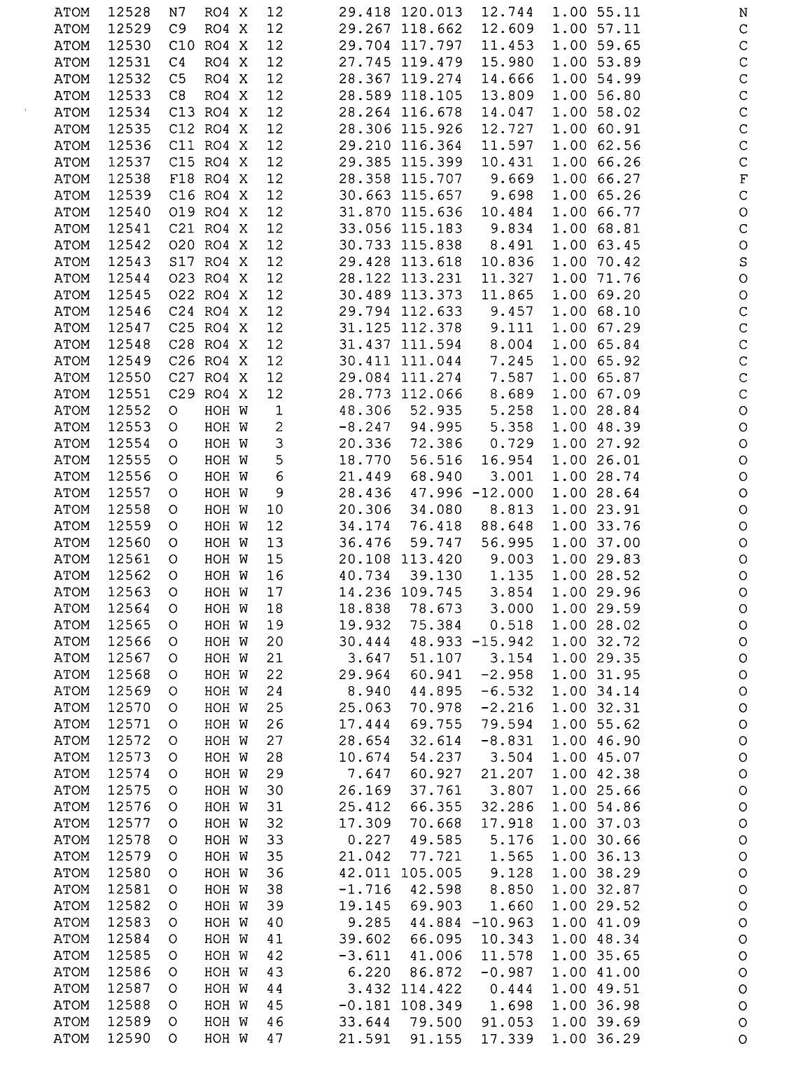

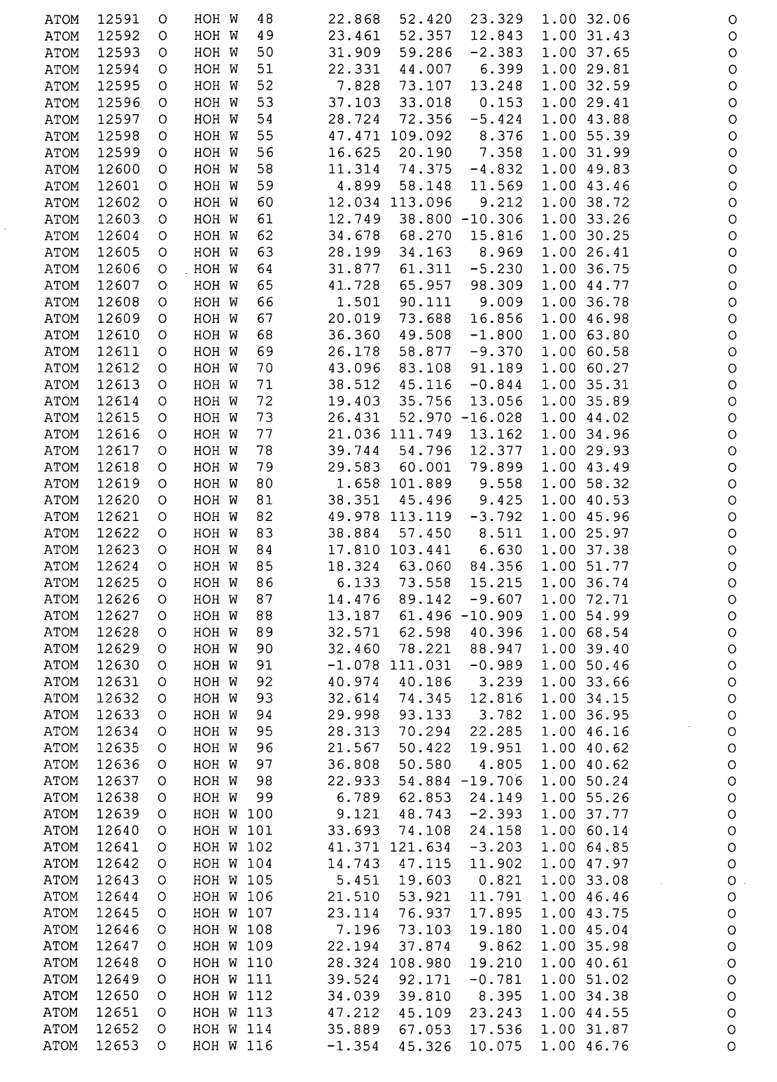

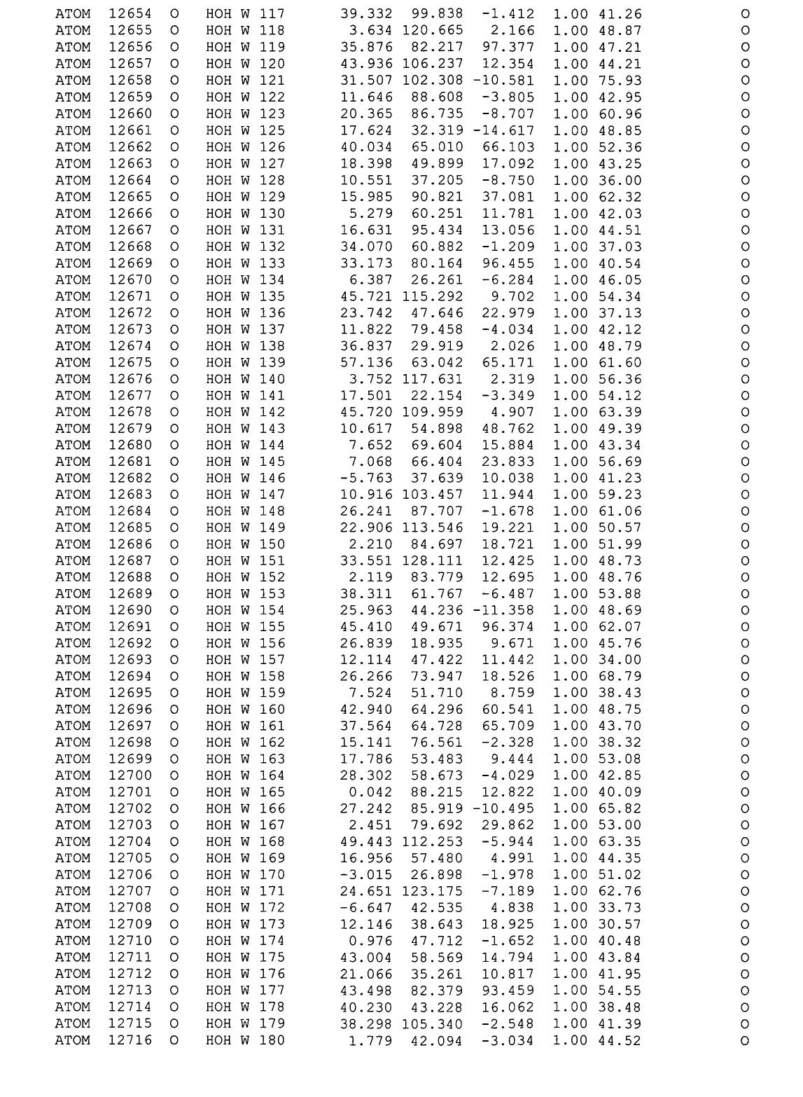

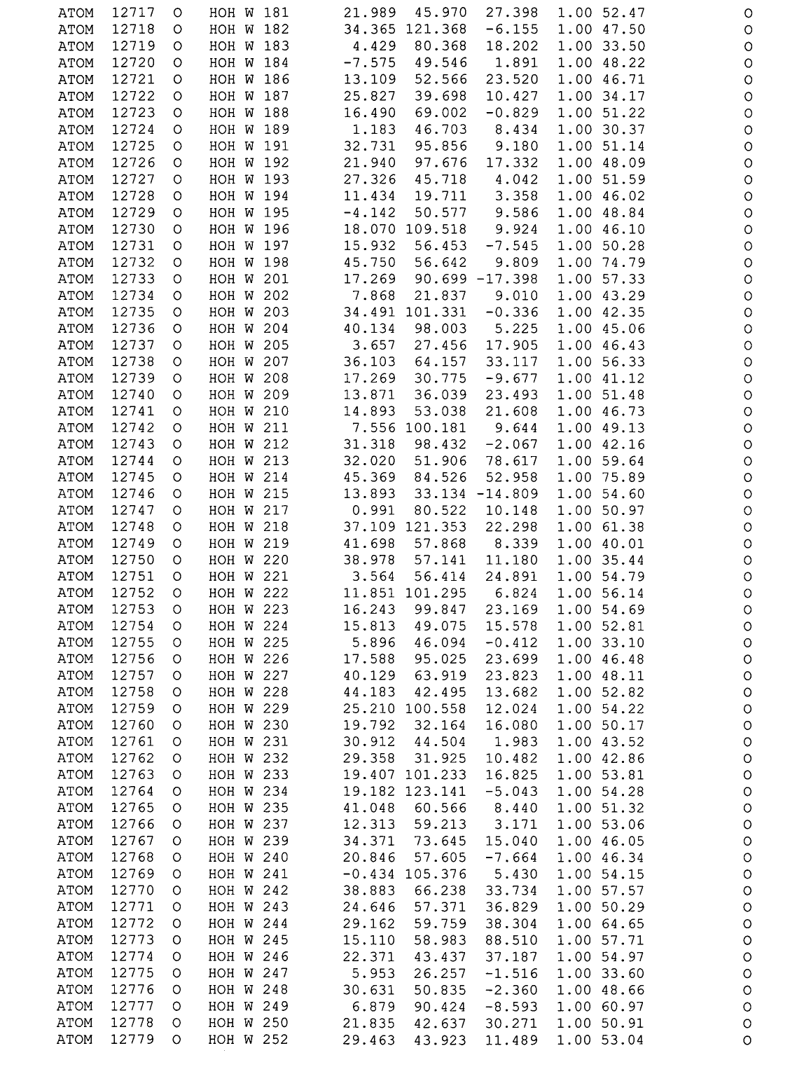

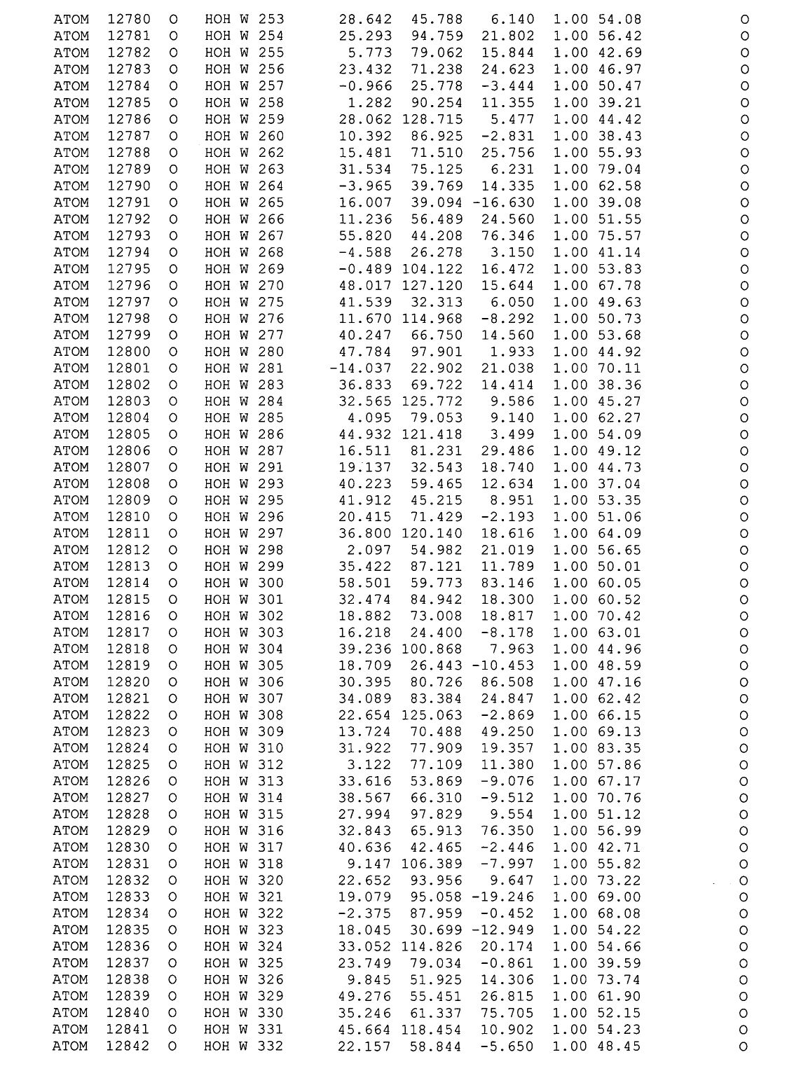

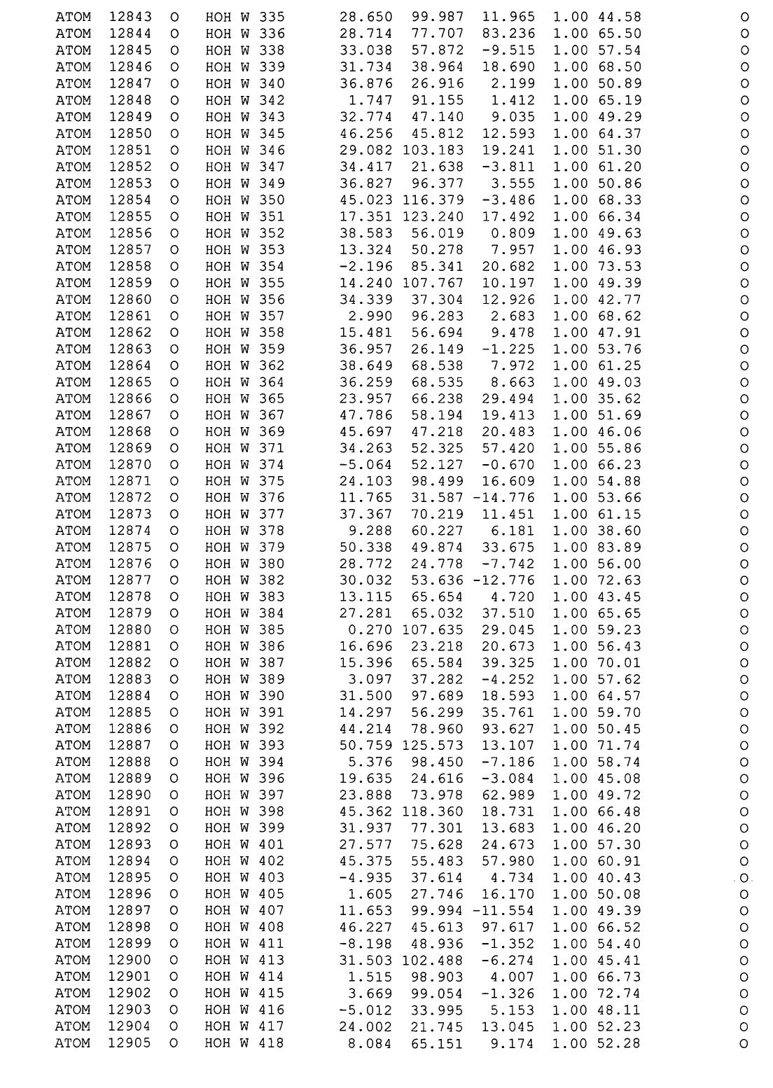

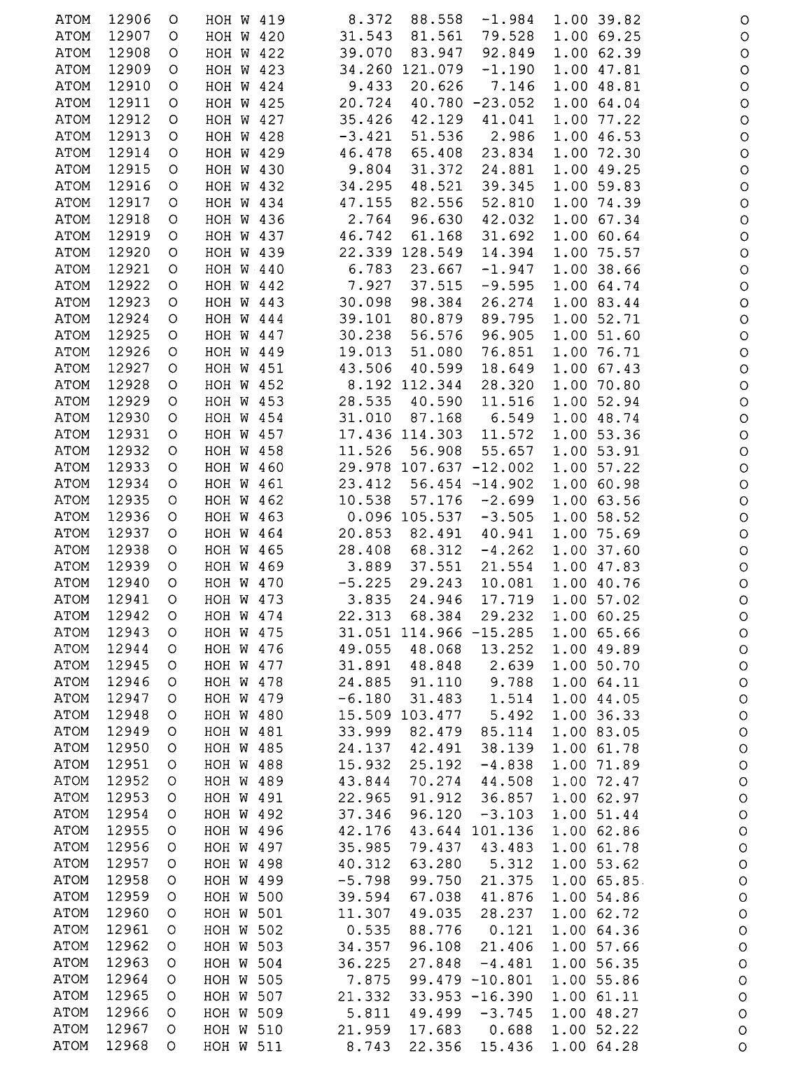

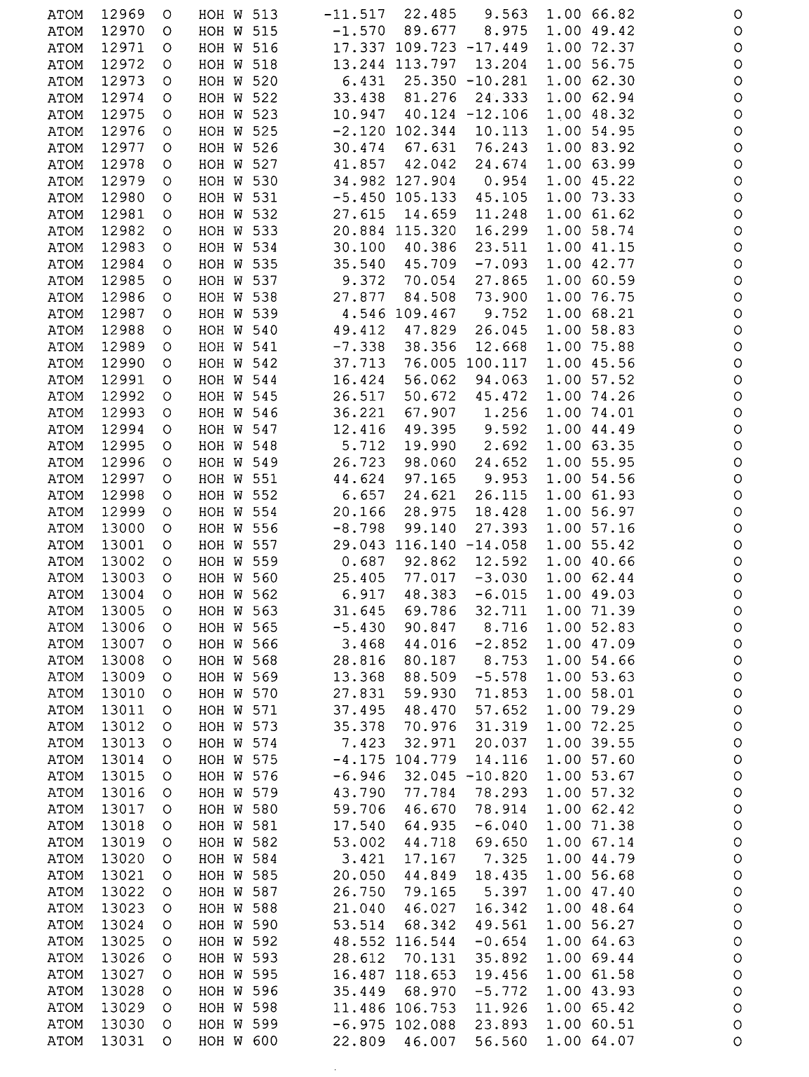

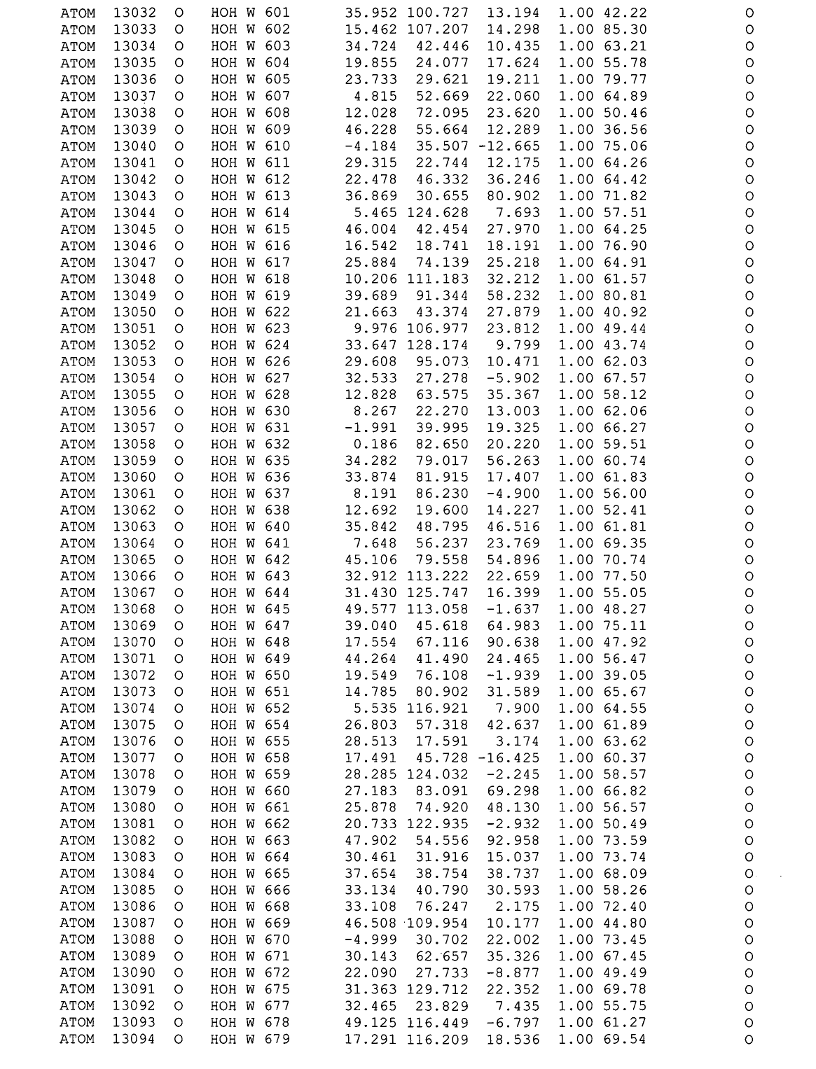

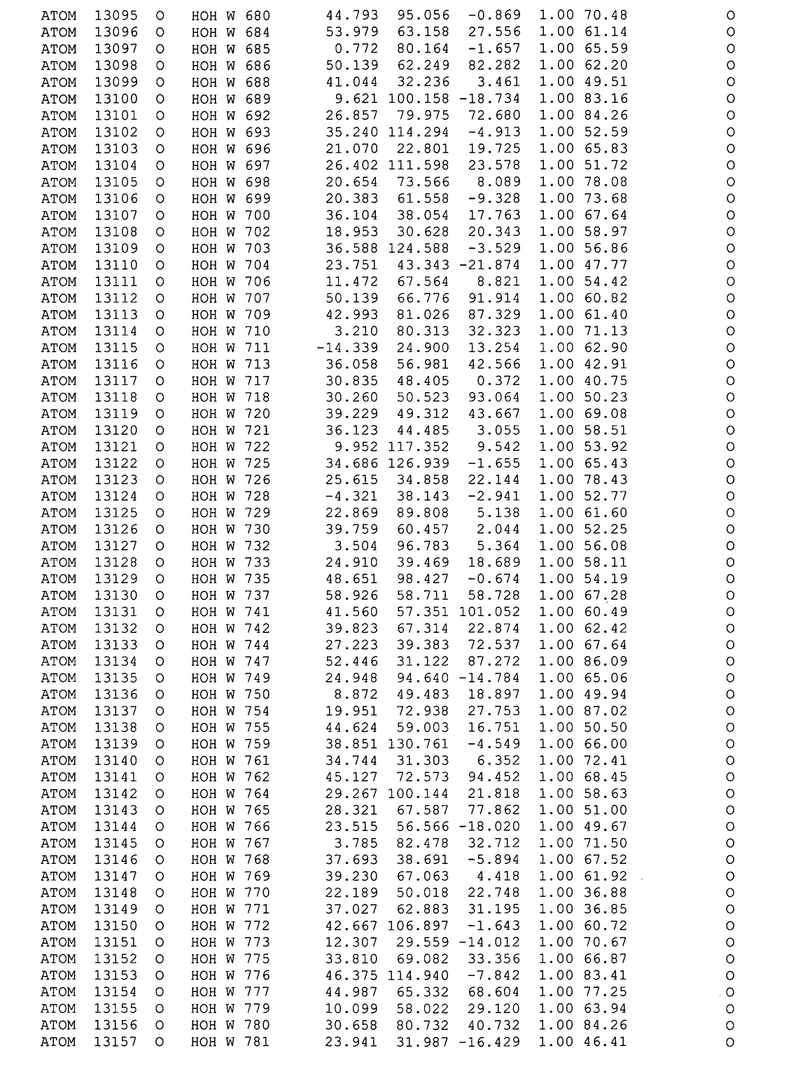

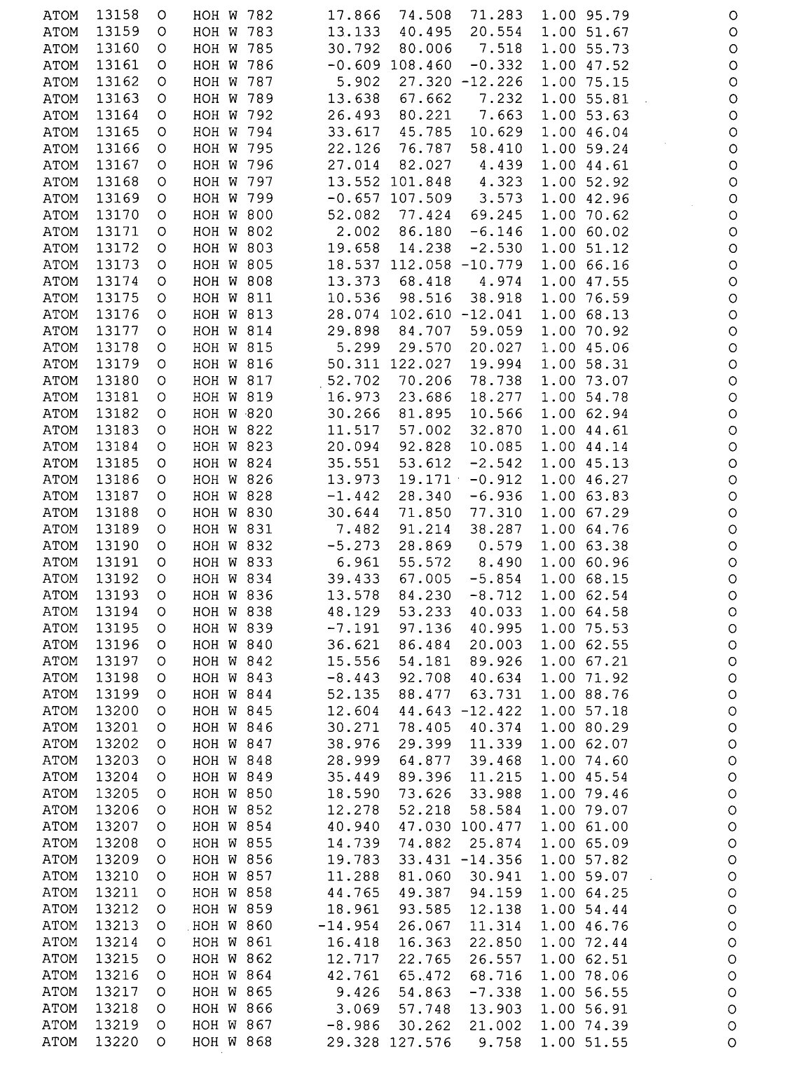

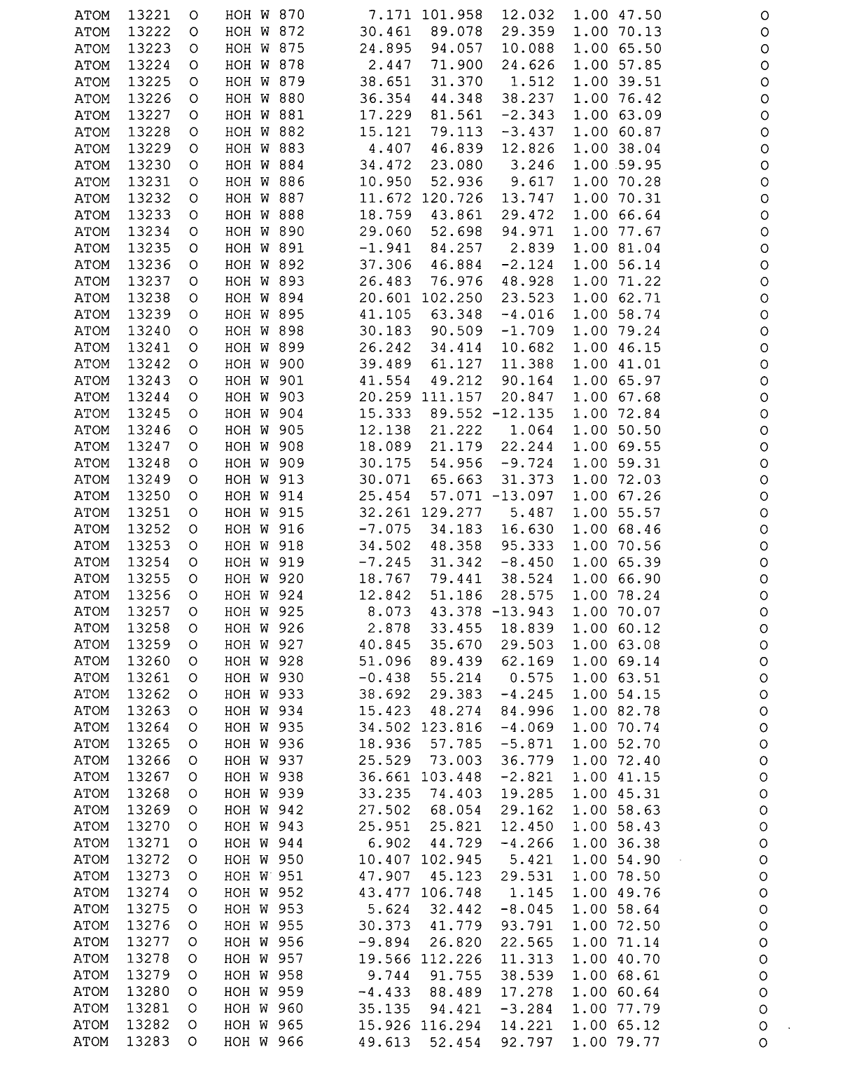

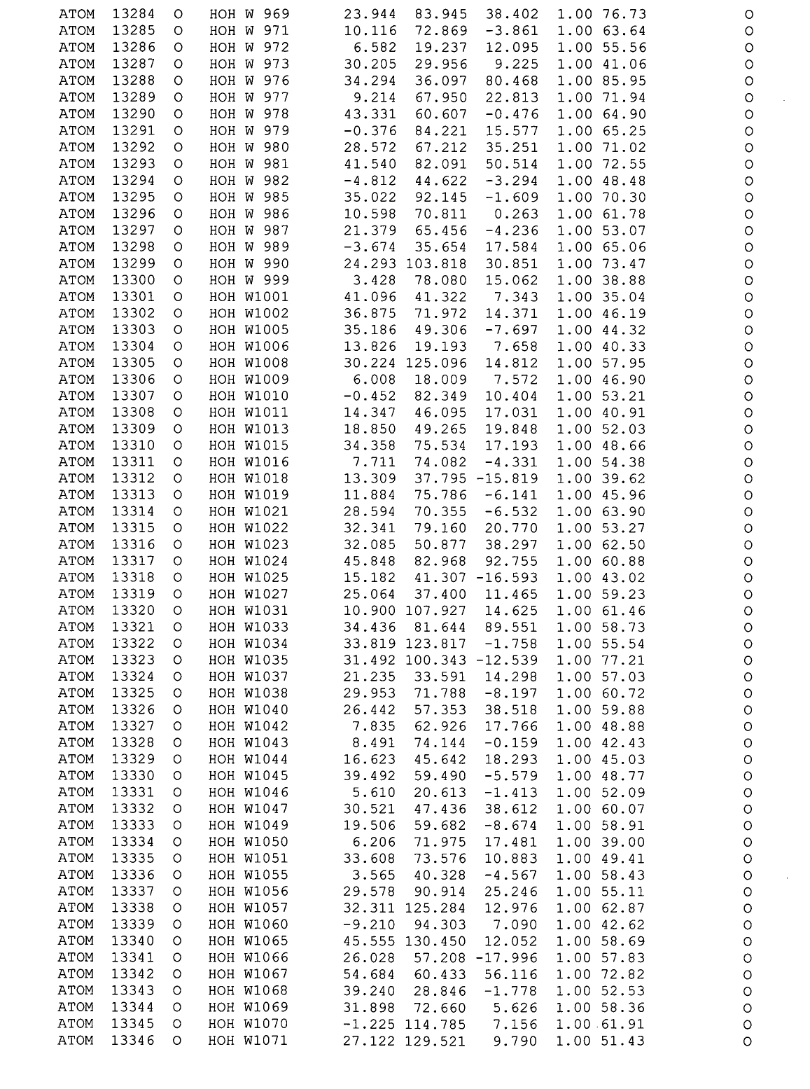

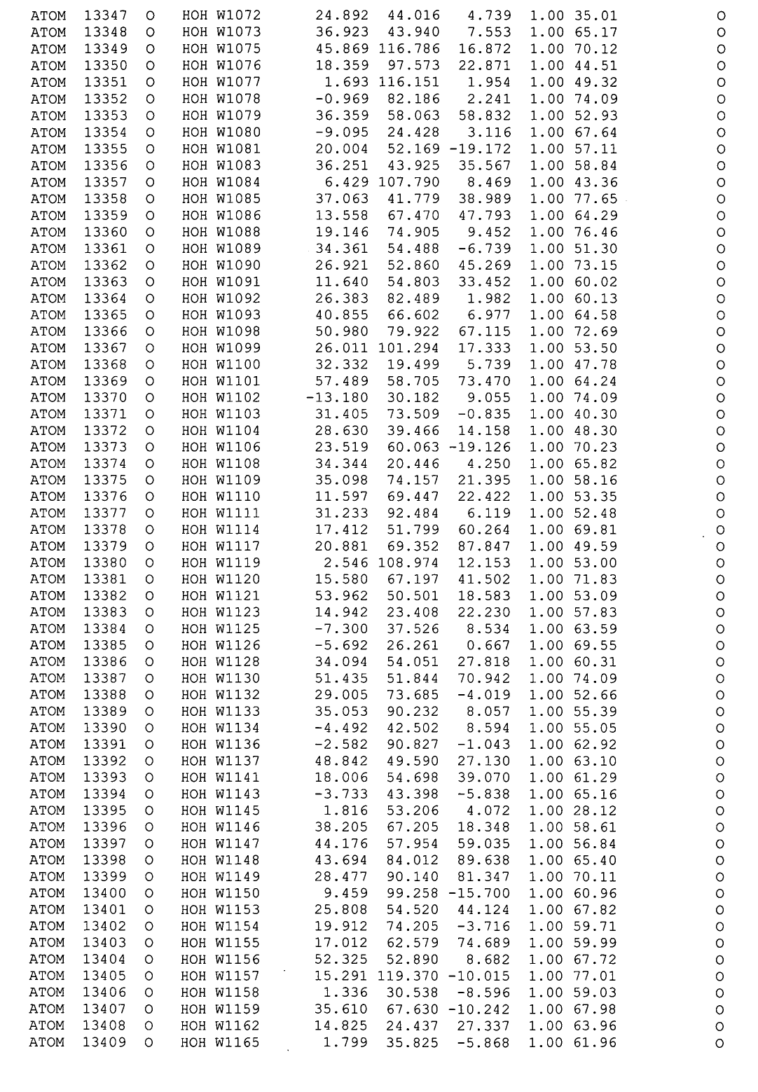

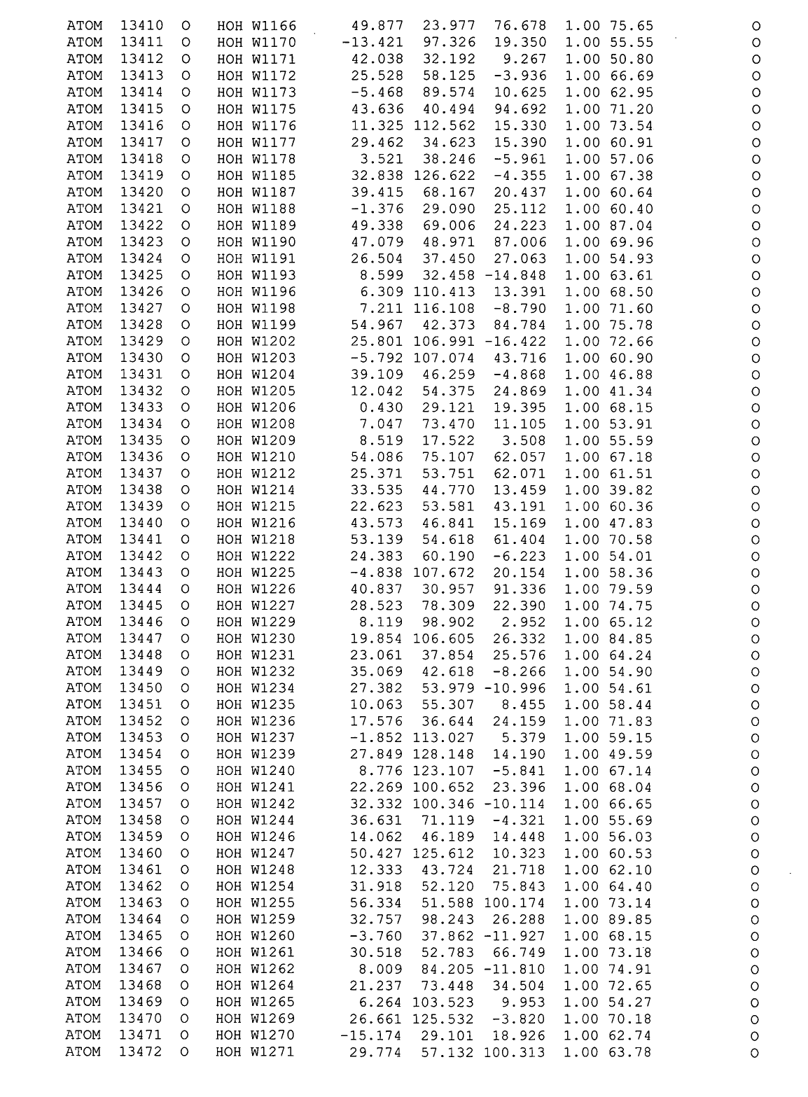

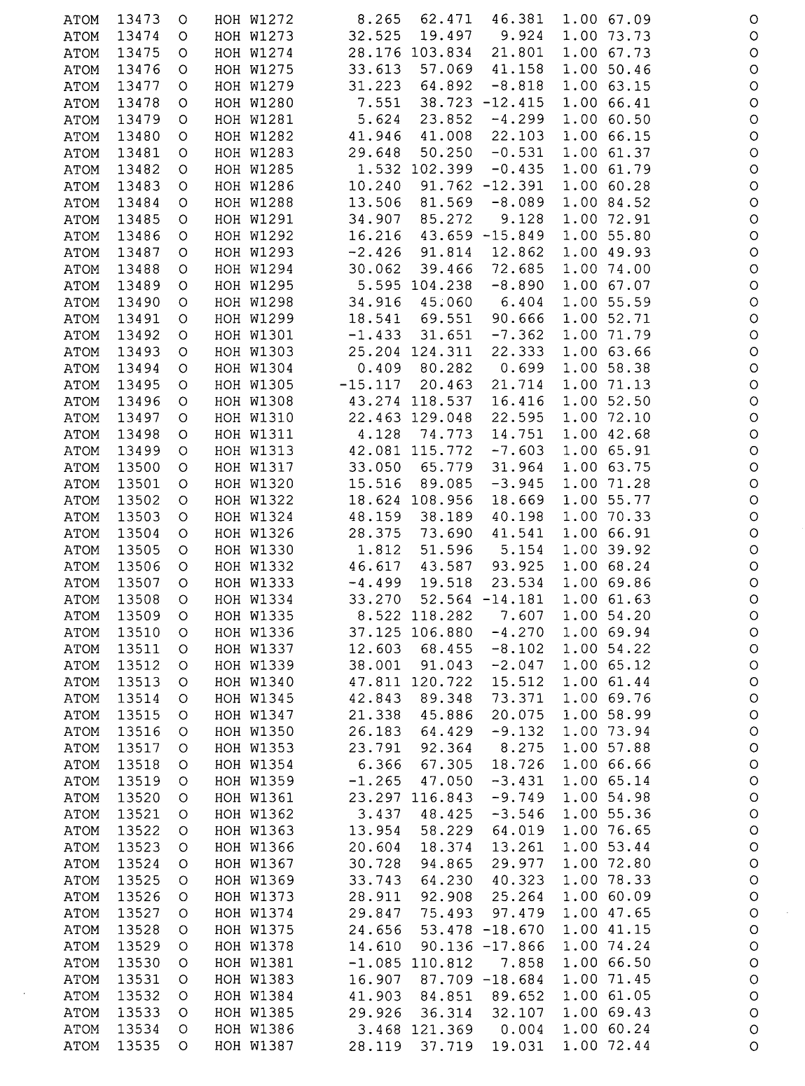

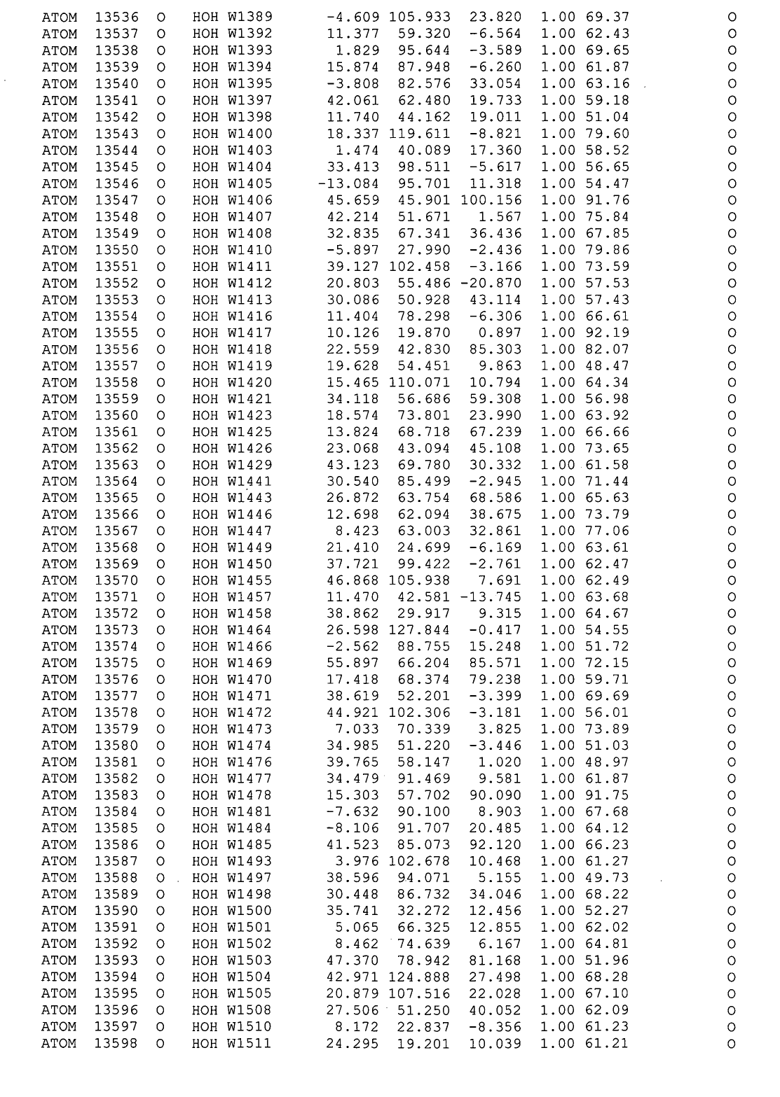

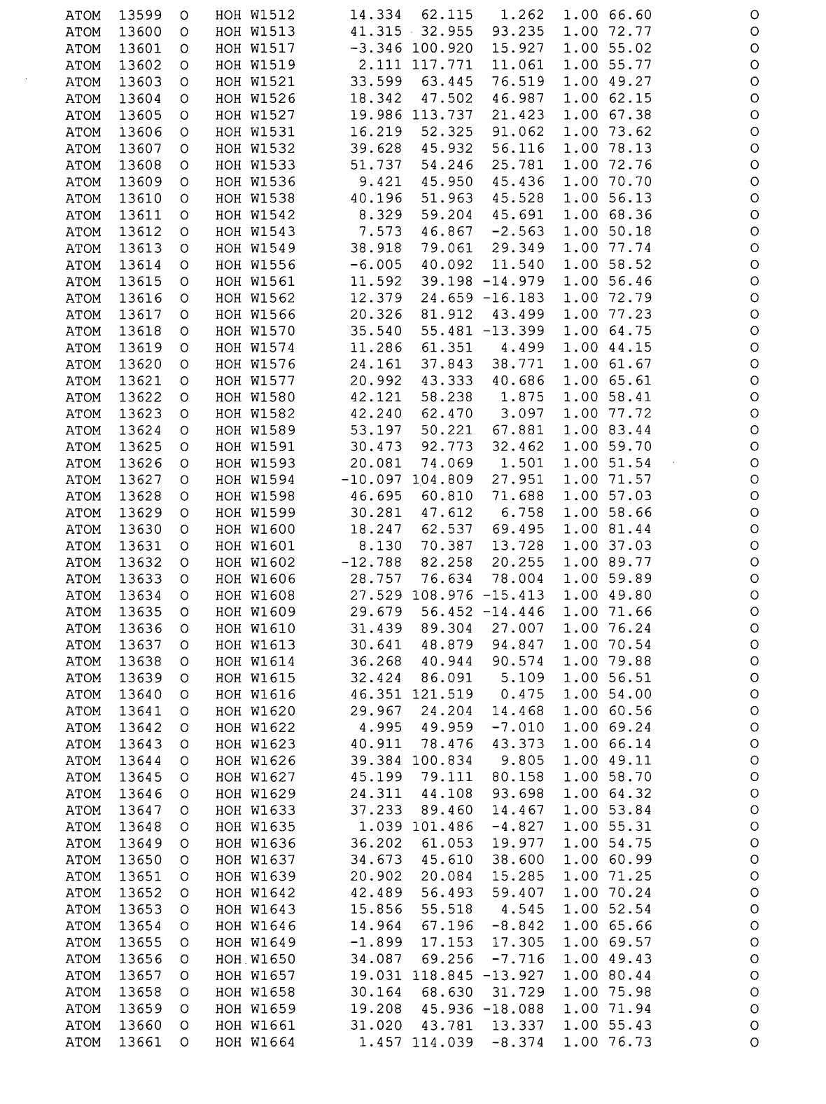

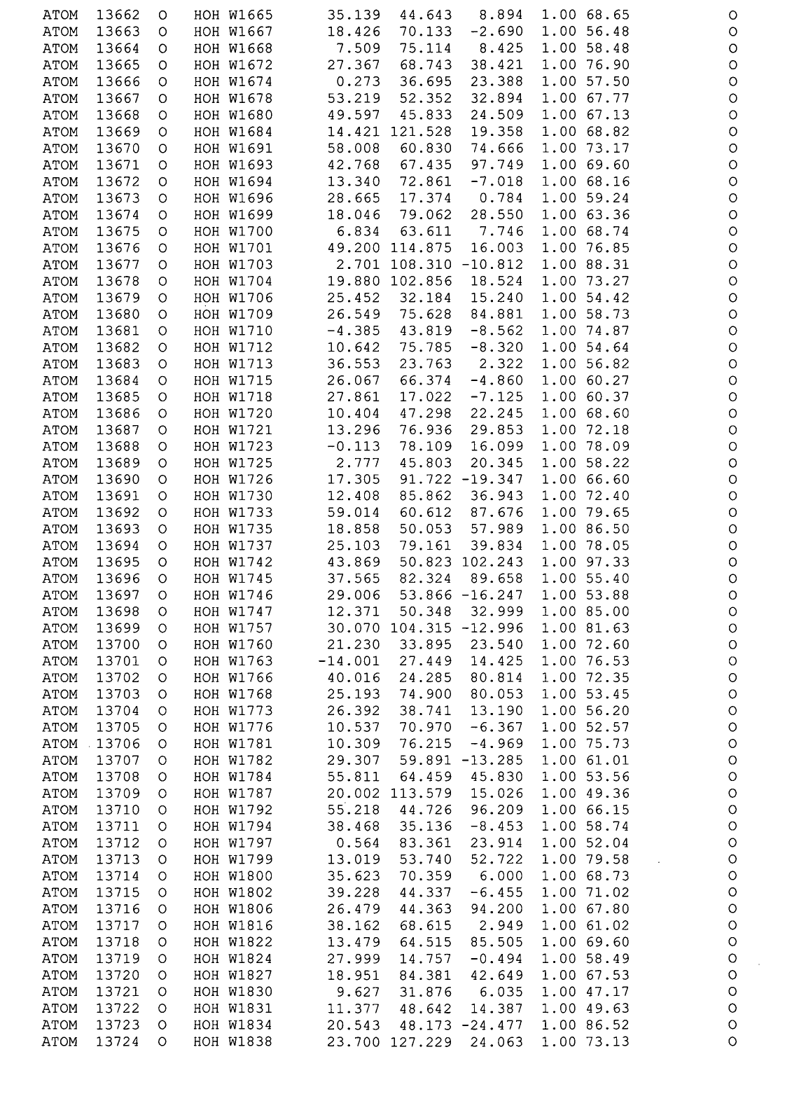

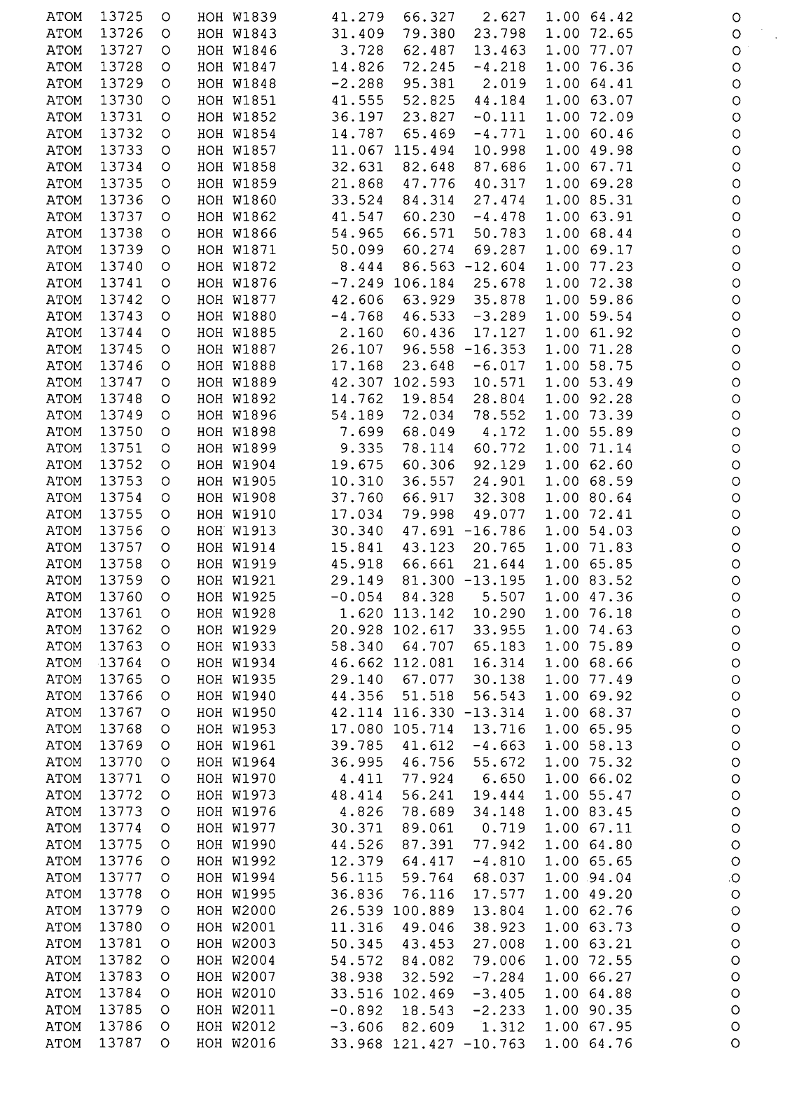

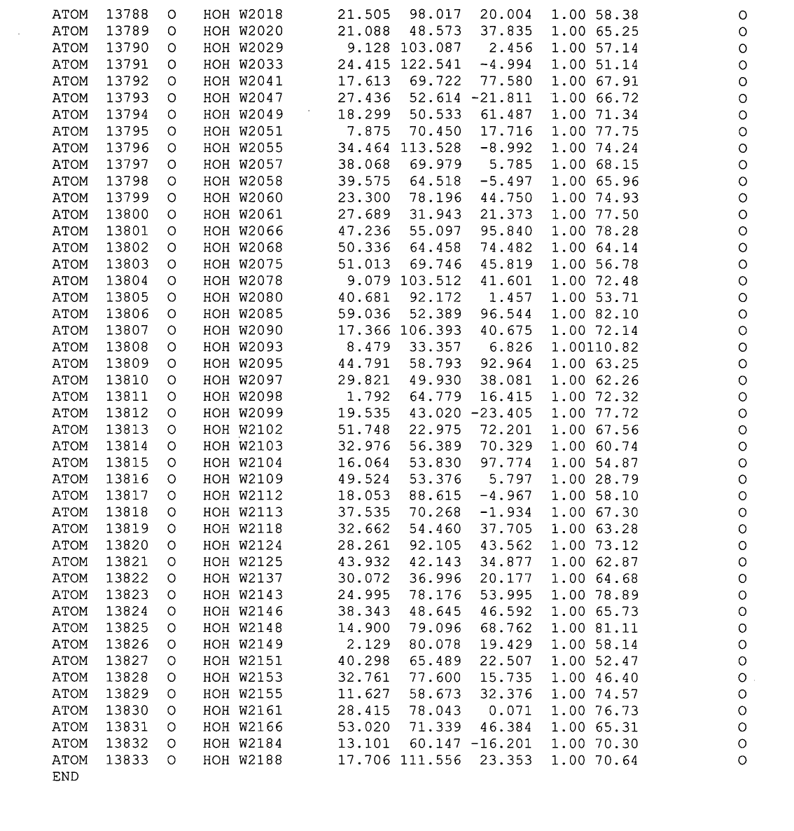

11. The co-crystal of claim 10, wherein the LXRβ-LBD polypeptide comprises amino acids 220-239, 263-459, 220-242, 263-459, 220-239, 267-459, 220-240, and 266-459 (FIG. 1), 220-239, 263-459, 220-242, 263-459, 220-239, 267-459, 220-240, and 266-459 (FIGS. 2) or 220-459, 220-459, 220-459, 220-242, 249-459, 220-459, and 220-459 (FIG. 3) of Seq. Id. No.1.

12. A method for co-crystalling a LXRβ-LBD polypeptide with a compound that binds to the Ligand binding site of said polypeptide, the method comprising:
a) providing an aqueous solution of the LXRβ-LBD polypeptide, wherein the polypeptide comprises a sequence having a sequence similarity to SEQ ID NO:1 of at least 80%
b) adding a molar excess of a Ligand to the aqueous solution of the polypeptide, and
c) growing crystals.

13. The method of claim 12, wherein the aqueous solution further additionally comprises 5% to 30% (w/v) PEG, wherein further the PEG has an average molecular weight of 200 Da to 20 kDa, preferably 200 Da to 5 kDa.

14. The method of claim 13, wherein the aqueous solution additionally comprises 0 M to 1 M Bis-tris pH 5.5, 0 M to 0.5 M magnesium chloride, and 0 M to .5 M ammonium sulfate.

15. The method of claim 14, wherein the aqueous solution further additionally comprises a molar excess of a co-activator peptide, preferably a 3-12 fold molar excess.

16. The method of claim 15, wherein the Ligand is added in a 12-15 molar excess.

17. A method for identifying a compound that can bind to the Ligand binding site of a LXRpolypeptide comprising the steps:
a) determining an active site of a LXR polypeptide form the three dimensional model of LXRβ-LBD polypeptide using the atomic coordinates of FIG. 1, FIG. 2 or FIG. 3, ± a root mean square deviation from the backbone atoms of said amino acids of not more than 2 Å; and
b) performing computer fitting analysis to identify a compound that can bind to the binding site of the LXR polypeptide.

18. The method of claim 17 comprising the steps:
a) generating a three dimensional model of a Ligand binding site of LXR polypeptide using the relative structural data coordinates of FIG. 1, 2 or 3 of residues GLN235, CYS238, ASN239, PHE243, PHE268, PHE271, THR272, LEU274, ALA275, SER278, GLU281, ILE282, PHE285, ILE309, ILE311, MET312, GLU315, THR316, ARG319, ILE327, THR328, PHE329, LEU330, TYR335, PHE340, LEU345, PHE349, ILE353, ILE374, HIS435, GLN438, VAL439, LEU442, LEU449 and TRP457±a root mean square deviation from the backbone atoms of said amino acids of not more than 2 Å; and
b) performing computer fitting analysis to identify a compound that can bind to the PDE10-cat active site.

19. A co-crystal of LXRβ-LBD containing LXRβ-LBD in a conformation defined by the coordinates of FIG. 1, FIG. 2 or FIG. 3, optionally varied by an rmsd of less than 2.0Å.

20. (canceled)

21. The co-crystal of claim 4, wherein the LXRβ-LBD polypeptide is a polypeptide comprising a sequence having a similarity to the Ligand binding domain of a polypeptide of Seq. Id. No. 1 of at least 85%, more preferably at least 90%, even more preferably at least 95%, most preferably 100%.

22. The co-crystal of claim 21, wherein the LXRβ-LBD polypeptide comprises amino acids 213-461 of Seq. Id. No. 1.

23. The co-crystal of claim 7, wherein the LXRβ-LBD polypeptide is a polypeptide comprising a sequence having a similarity to the Ligand binding domain of a polypeptide of Seq. Id. No. 1 of at least 85%, more preferably at least 90%, even more preferably at least 95%, most preferably 100%.

24. The co-crystal of claim 22, wherein the LXRβ-LBD polypeptide comprises amino acids 213-461 of Seq. Id. No. 1.
